(19) United States
(12) Patent Application Publication
(10) Pub. No.: US 2019/0322676 A1
(43) Pub. Date: Oct. 24, 2019

(54) CERAMIDE GALACTOSYLTRANSFERASE INHIBITORS FOR THE TREATMENT OF DISEASE

(71) Applicant: BioMarin Pharmaceutical Inc., Novato, CA (US)

(72) Inventor: Bing WANG, San Jose, CA (US)

(21) Appl. No.: 16/471,515

(22) PCT Filed: Dec. 19, 2017
(86) PCT No.: PCT/US2017/067184

§ 371 Date: Jun. 19, 2019

Related U.S. Application Data
(60) Provisional application No. 62/437,011, filed on Dec. 20, 2016.

(51) Int. Cl. C07D 487/08; C07D 487/10; C07D 451/04; C07D 211/26; C07D 207/14; C07D 211/58; C07D 487/04; C07D 409/12; C07D 241/38; C07D 405/06

(52) U.S. Cl. CPC C07D 487/08; C07D 487/10; C07D 451/04; C07D 211/26; C07D 405/06; C07D 211/58; C07D 487/04; C07D 409/12; C07D 241/38; C07D 207/14

(57) ABSTRACT

Described herein are compounds, methods of making such compounds, pharmaceutical compositions and medicaments containing such compounds, and methods of using such compounds to treat or prevent diseases or disorders associated with the enzyme ceramide galactosyltransferase (CGT), such as, for example, lysosomal storage diseases. Examples of lysosomal storage diseases include, for example, Krabbe disease and Metachromatic Leukodystrophy.

### CROSS REFERENCE

This application claims the benefit of priority of U.S. Provisional Patent Application No. 62/437,011, filed Dec. 20, 2016, the content of which is incorporated herein by reference in its entirety.

### FIELD

Described herein are compounds, methods of making such compounds, pharmaceutical compositions and medicaments containing such compounds, and methods to treat or prevent diseases or disorders associated with the enzyme ceramide galactosyltransferase (CGT). Also described herein is that such compounds are for use in said methods for treating or preventing diseases or disorders. Such diseases or disorders include, for example, lysosomal storage diseases (LSDs). Examples of lysosomal storage diseases include Krabbe disease and Metachromatic Leukodystrophy.

### BACKGROUND

Ceramide galactosyltransferase (CGT) is a key enzyme in glycosphingolipid (GSL) biosynthesis in eukaryotic cells. Glycosphingolipids (GSLs) are believed to be integral in many cell membrane events, including cellular interactions, signaling, and trafficking. Ceramides play a central role in sphingolipid metabolism, and CGT facilitates conversion of ceramides to galactosylceramides. Galactosylceramides can be further modified by the enzyme cerebroside sulfotransferase (CST) to form sulfatides. Galactosylceramides and sulfatides are primarily produced by the myelin generating cells of the central and peripheral nervous systems, oligodendrocytes and Schwann cells respectively, where these glycolipids make up a large proportion of the lipids in the myelin sheath. Galactosylceramide and sulfatide are also found on the extracellular leaflet of the plasma membrane of other cells in eukaryotic organisms where they have been reported to be involved in a diverse range of functions.

Degradation of galactosylceramides is catalyzed in the lysosome by galactosylceramidase (GALC). Insufficient degradation of galactosylceramides, caused by deficiency of GALC, can lead to an accumulation of galactosylceramides and its partially degraded product psychosine (also called galactosylsphingosine). In humans, deficiency of GALC results in Krabbe disease (also known as globoid cell leukodystrophy or galactosylceramide lipidosis). See, e.g., Ezoe et al., J. Neurosci. Res. 59:170-178 (2000); Ezoe et al., J. Neurosci. Res. 59:179-187 (2000). Increased psychosine levels are believed to be the primary toxic molecule in Krabbe disease leading to widespread destruction of oligodendrocytes in the CNS and Schwann cells in the PNS and subsequent demyelination. See, e.g., Suzuki et al., Proc. Natl. Acad. Sci. U.S.A. 66(2):302-9 (1970); Graziano et al., Gene 555(1):2-13 (2015). Similarly, turnover of sulfatides occurs in lysosome via the enzyme arylsulfatase A (ASA) and defects in ASA can lead to the accumulation of sulfatides and its partially degraded product lyso-sulfatides. ASA deficiency can lead to the development of an autosomal recessive disease called metachromatic leukodystrophy (MLD). See, e.g., Kohlschitter, Handb. Clin. Neurol. 113:1611-1618 (2013).

Krabbe disease detrimentally affects the myelin sheath, which protects nerves and facilitates the sending and receiving of nerve signals. The accumulation of unmetabolized galactosylceramides and psychosine detrimentally affects the growth and development of the myelin sheath. Damage to the myelin sheath can lead to a severe degeneration of motor skills, cognitive deficits, and seizures, and is often fatal. Similarly, in MLD, accumulation of sulfatides and lyso-sulfatides detrimentally affects the myelin sheath, disrupting neuronal functions and leads to seizures, progressive coordination and speech problems, and other behavioral disturbances.

An approach to treatment of such diseases resulting from an abnormal accumulation of galactosylceramides, psychosine, sulfatides, lyso-sulfatides and related GSLs is to inhibit the CGT enzyme to reduce the synthesis of galactosylceramides and other downstream molecules. Accordingly, molecules that inhibit the activity of CGT are useful as therapeutic agents in the treatment of lysosomal storage diseases relating to defects in sphingolipid metabolism, such as Krabbe disease and MLD. In addition, abnormal metabolism of galactosylceramides and sulfatides has been associated with other pathological conditions such as Parkinson's Disease. See, e.g., Marshall and Bongarzone, J. Neurosci. Res., 94:1328 (2016); Smith et al., J. Pathol. 232:509 (2014). Overexpression of sulfatide has also been linked to epilepsy and audiogenic seizures as well as other pathological states in the nervous system. Accordingly, molecules that inhibit the activity of CGT may be used to treat Parkinson's disease, epilepsy and audiogenic seizures that are associate with overexpression or accumulation of galactosylceramides/sulfatides.

### SUMMARY

In one aspect, provided herein is a compound of Formula I:

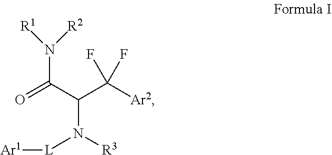

where

R¹ is alkyl, aminoalkyl, alkylaminoalkyl, or dialkylaminoalkyl;

R² is alkyl, aminoalkyl, alkylaminoalkyl, or dialkylaminoalkyl;

or R¹ and R² together with the nitrogen to which they are attached form a 4-8 membered heterocycloalkyl ring optionally substituted with 1, 2, or 3 R¹⁰ groups; or a bicyclic heteroaryl ring with 8 ring atoms, where 1-3 ring atoms are nitrogen;

R³ is H, alkyl, or haloalkyl; L is S(O)_{q}, C₁₋₃alkylene-S(O)_{q}, C(O)—C(O), or C*H(OH)C(O), wherein the alkylene in the C₁₋₃alkylene-S(O) q forms a covalent bond with Ar¹, and wherein the “C*” in the C*H(OH)C(O) forms a covalent bond with Ar¹;

q is 0, 1, or 2;

Ar¹ is aryl or heteroaryl, each optionally substituted with 1 or 2 R¹¹ groups;

Ar² is aryl or heteroaryl, each optionally substituted with 1 or 2 R¹² groups;

each R¹⁰ is independently halo, cyano, nitro, amino, alkylamino, dialkylamino, aminoalkyl, alkylaminoalkyl, dialkylaminoalkyl, aminocarbonyl, alkylaminocarbonyl, dialkylaminocarbonyl, or C(═NOR^{10a})(NR^{10b}R^{10c});

R^{10a}, R^{10b}, and R^{10c} are each independently H or alkyl;

each R¹¹ is independently halo, cyano, nitro, alkyl, alkoxy, hydroxy, cycloalkyl, (cycloalkyl)alkyl, heterocycloalkyl, (heterocycloalkyl)alkyl, cycloalkyloxy, heterocycloalkyloxy, (cycloalkyl)alkyloxy, cycloalkyloxyalkyl, aryl, aryloxy, arylalkyloxy, heteroaryl, heteroaryloxy, or heteroarylalkyloxy; wherein each aryl and heteroaryl, whether alone or as part of another group, are each independently optionally substituted with halo, alkyl, haloalkyl, or alkoxy;

each R¹² is independently halo, cyano, nitro, hydroxy, alkyl, haloalkyl, haloalkenyl, alkoxy, cycloalkyloxy, cycloalkyl, heterocycloalkyl, cycloalkyl substituted with haloalkyl, (cycloalkyl)alkyloxy, heterocycloalkyloxy, (heterocycloalkyl)alkyloxy, aryl, heteroaryl, aryloxy, arylalkyloxy, heteroaryloxy, or heteroarylalkyloxy; wherein each aryl and heteroaryl, whether alone or as part of another group, are each independently optionally substituted with 1 or 2 groups selected from halo, alkyl, haloalkyl, and alkoxy; and

optionally a single stereoisomer or mixture of stereoisomers thereof and additionally optionally a pharmaceutically acceptable salt thereof.

In another aspect, provided herein is a pharmaceutical composition comprising a compound disclosed herein, for example, a compound of Formula (I), (Ia), (Ib), I(c), I(d), I(e), I(f), II(a), II(b), II(c), II(d), II(e), II(f), II(g), II(h), III(a), III(b), IV(a), IV(b), IV(c), IV(d), V(a), V(b), V(c), or V(d), as disclosed herein, and a pharmaceutically acceptable excipient.

In a further aspect, provided herein is a method of treating lysosomal storage disease with a compound disclosed herein. Thus, a compound disclosed herein is for use in a method of treating lysosomal storage disease. Such a compound is, for example, a compound of Formula (I), (Ia), (Ib), I(c), I(d), I(e), I(f), II(a), II(b), II(c), II(d), II(e), II(f), II(g), II(h), III(a), III(b), IV(a), IV(b), IV(c), IV(d), V(a), V(b), V(c), or V(d), as disclosed herein, or a pharmaceutical composition disclosed herein.

In certain embodiments, the lysosomal storage disease is Krabbe disease or Metachromatic Leukodystrophy.

### DETAILED DESCRIPTION

### Abbreviations

| Abbreviation | Meaning |
|---|---|
| ASA | arylsulfatase A |
| Boc₂O | di-tert-butyl dicarbonate |
| CbzCl | benzyl chloroformate |
| CGT | ceramide galactosyltransferase |
| DCM | dichloromethane |
| DAST | diethylaminosulfur trifluoride |
| DEA | diethanolamine |
| DIPEA | diisoproylethylamine |
| DMF | dimethylformamide |
| DMSO | dimethyl sulfoxide |
| EDCI | 1-ethyl-3-(3-dimethylaminopropyl)carbodiimide |
| ESI | electrospray ionization |
| EtOH | ethanol |
| Et₂O | diethyl ether |
| Et₃N | triethylamine |
| GALC | galactosylceramidase |
| GSL | glycosphingolipids |
| HATU | O-(7-azabenzotriazol-1-yl)-N,N,N′,N′-tetramethyluronium |
| | hexafluorophosphate |
| HPLC | high performance liquid chromatography |
| Hz | Hertz (s⁻¹) |
| LC-MS | liquid chromatography-mass spetrometry |
| mg | milligram |
| MeOH | methanol |
| M-PER | Mammalian Protein Extraction Reagent |
| MHz | MegaHertz |
| mL | milliliter |
| mm | millimeter |
| μm | micrometer |
| mmol | millimole |
| MLD | metachromatic leukodystrophy |
| MsCl | methanesulfonyl chloride (mesyl chloride) |
| μL | microliter |
| mM | millimolar |
| μM | micromolar |
| n-BuLi | n-butyllithium |
| NMP | N-methyl pyrrolidone |
| NMR | Nuclear Magnetic Resonance |
| ppm | parts per million |
| POCl₃ | phosphoryl chloride |
| PPTS | pyridinium p-toluenesulfonate |
| TEA | triethanolamine |
| THF | tetrahydrofuran |
| TMS | trimethylsilane |

### Definitions

To facilitate understanding of the disclosure set forth herein, a number of terms are defined below. Generally, the nomenclature used herein and the laboratory procedures in organic chemistry, medicinal chemistry, and pharmacology described herein are those well-known and commonly employed in the art.

“About” preceding a numerical value refers to a range of values±10% of the value specified.

“Acceptable” with respect to a formulation, composition or ingredient, means having no persistent detrimental effect on the general health of the subject being treated.

Whenever a group is described as being “optionally substituted,” the group can be unsubstituted or substituted.

“Alkenyl” means a straight or branched hydrocarbon radical having from 2 to 8 carbon atoms and at least one double bond and certain embodiments include ethenyl, propenyl, 1-but-3-enyl, 1-pent-3-enyl, or 1-hex-5-enyl.

“Alkoxy” means a group of the formula —OR, where R is alkyl. In certain embodiments, alkoxy includes methoxy, ethoxy, propoxy, 2-propoxy, butoxy, tert-butoxy, pentyloxy, or hexyloxy.

“Alkyl” means a straight or branched saturated hydrocarbon radical containing from 1-10 carbon atoms, and in certain embodiments includes 1-6 carbon atoms (“C₁₋₆ alkyl”), in certain embodiments includes 1-4 carbon atoms (“C₁₋₄ alkyl”), and in certain embodiments includes 1-3 carbon atoms (“C₁₋₃ alkyl”). In certain embodiments, alkyl includes methyl, ethyl, n-propyl, iso-propyl, n-butyl, sec-butyl, tert-butyl, n-pentyl, isopentyl, neopentyl, n-hexyl, 3-methylhexyl, 2,2-dimethylpentyl, 2,3-dimethylhexyl, n-heptyl, n-octyl, n-nonyl, or n-decyl.

“Alkylamino” means a group of the formula —NHR, where R is alkyl. In certain embodiments, alkylamino includes methylamino, ethylamino, n-propylamino, iso-propylamino, n-butylamino, iso-butylamino, or tert-butylamino.

“Alkylaminoalkyl” means an alkyl group substituted with one or two alkylamino groups.

“Alkylene” refers to a divalent radical formed by removal of a hydrogen atom from alkyl.

“Aminoalkyl” means an alkyl group substituted with at least one amino group, and in certain embodiments, with one, two, or three amino groups.

“Alkylaminocarbonyl” means a group of the formula —C(O)R, where R is alkylamino.

“Amino” means an —NH₂ group.

“Aminocarbonyl” means a —C(O)NH₂ group.

“Aryl” means a monovalent six- to fourteen-membered, mono-, bi-, or tri-carbocyclic ring, wherein the monocyclic ring is aromatic and at least one of the rings in the bicyclic or tricyclic ring is aromatic. In certain embodiments, aryl includes phenyl, naphthyl, indanyl, or anthracenyl.

“Arylalkyloxy” means a group of the formula —O—R—R′, where R is alkylene and R′ is aryl. In certain embodiments, arylalkyloxy includes benzyloxy or phenethyloxy.

“Aryloxy” means a group of the formula —OR, where R is aryl. In certain embodiments, aryloxy includes phenoxy.

“Cycloalkyl” means a monocyclic or bicyclic, saturated or partially unsaturated (but not aromatic), hydrocarbon radical of three to ten carbon ring atoms. Cycloalkyl groups include fused, bridged and spirocycloalkyl bicyclic rings. For example, when fused, the cycloalkyl group may comprise two rings that share adjacent atoms (e.g., one covalent bond). When bridged, the cycloalkyl group may comprise two rings that share three or more atoms, separating the two bridgehead atoms by a bridge containing at least one atom. When spiro, the cycloalkyl group may comprise two rings that share only one single atom, the spiro atom, which may be, for example, a quaternary carbon. When a cycloalkyl group contains from 3-10 ring carbon atoms, it may be referred to herein as C₃₋₁₀ cycloalkyl. When a cycloalkyl group contains from 5-6 ring carbon atoms, it may be referred to herein as C₅₋₆ cycloalkyl.

In certain embodiments, cycloalkyl groups include cyclopropyl, cyclobutyl, cyclopentyl, or cyclohexyl. In certain embodiments, cycloalkyl groups include:

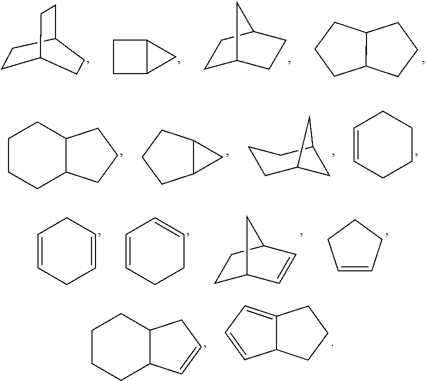

“(Cycloalkyl)alkyl” means an alkyl group substituted with at least one cycloalkyl group. In certain embodiments, the alkyl is substituted with 1 or 2 cycloalkyl groups. In certain embodiments, alkyl is substituted with 1 cycloalkyl group. In certain embodiments, (cycloalkyl)alkyl includes cyclobutylmethyl, cyclopentylmethyl, and cyclohexylmethyl.

“(Cycloalkyl)alkyloxy” means a group of the formula —OR, where R is a (cycloalkyl)alkyl group. In certain embodiments, (cycloalkyl)alkyloxy includes cyclobutylmethoxy, cyclopentylmethoxy, and cyclohexylmethoxy.

“Cycloalkyloxy” means a group of the formula —OR, where R is cycloalkyl. In certain embodiments, cycloalkyloxy includes cyclobutyloxy, cyclopentyloxy, and cyclohexyloxy.

“Cycloalkyloxyalkyl” means a group of the formula —R—O—R′, where R is alkylene and R′ is cycloalkyl.

“Dialkylamino” means a group of the formula —NRR′, where R and R′ are independently alkyl. In certain embodiments, dialkylamino includes dimethylamino, diethylamino, N,N-methylpropylamino or N,N-methylethylamino.

“Dialkylaminoalkyl” means an alkyl group substituted with at least one dialkylamino group. In certain embodiments, alkyl group is substituted with one or two dialkylamino group(s).

“Dialkylaminocarbonyl” means a group of the formula —C(O)R, where R is dialkylamino.

“Halo” means a fluoro, chloro, bromo, or iodo group.

“Haloalkoxy” means an alkoxy group, substituted with one or more halo atoms, and in certain embodiments by 1, 2, or 3 halo atoms. Certain embodiments of haloalkoxy include difluoromethoxy, trifluoromethoxy, or 1,1,1-trifluoroethoxy.

“Haloalkyl” means an alkyl group substituted with one or more halo atoms, and in certain embodiments by 1, 2, 3, 4, 5, or 6 halo atoms, and in certain embodiments by 1, 2, or 3 halo atoms, and in certain embodiments by 1 halo atom. In certain other embodiments, haloalkyl is an alkyl group substituted with 2 halo atoms. Certain embodiments of haloalkyl include chloromethyl, fluoromethyl, trifluoromethyl, or 1,1,1-trifluoroethane.

“Haloalkenyl” means an alkenyl group substituted with one or more halo atoms, and in certain embodiments by 1, 2, 3, 4, 5, or 6 halo atoms, and in certain embodiments by 1, 2, or 3 halo atoms, and in certain embodiments by 1 halo atom. Certain embodiments haloalkenyl include 3,3,3-trifluoroprop-1-enyl.

“Heteroaryl” means a monocyclic, fused bicyclic, or fused tricyclic ring of 5 to 14 ring atoms containing one or more ring heteroatoms independently selected from —O—, —S(O)ₙ— (n is 0, 1, or 2), —N=(trivalent nitrogen), —N(H)—, and N-oxide, and the remaining ring atoms being carbon, wherein the monocyclic ring is aromatic and wherein at least one of the rings in the bicyclic or tricyclic rings is aromatic (but does not have to be a ring which contains a heteroatom, e.g. 2,3-dihydrobenzo[b][1,4]dioxin-6-yl). Unless stated otherwise, the valency may be located on any atom of any ring of the heteroaryl group, valency rules permitting. In certain embodiments, heteroaryl contains one, two, three, or four ring heteroatoms.

In certain embodiments, heteroaryl includes, but is not limited to, triazolyl, tetrazolyl, pyrrolyl, imidazolyl, thienyl, furanyl, pyrazolyl, oxazolyl, isooxazolyl, oxadiazolyl, thiadiazolyl, indolyl, 2,3-dihydro-1H-indolyl (including, for example, 2,3-dihydro-1H-indol-2-yl or 2,3-dihydro-1H-indol-5-yl, and the like), indazolyl, phthalimidyl, benzimidazolyl, benzoxazolyl, benzofuranyl, dibenzo[b,d]furanyl, benzothienyl, benzopyranyl, benzothiazolyl, pyridinyl, pyrazinyl, pyrimidinyl, pyridazinyl, quinolinyl, isoquinolinyl, tetrahydroisoquinolinyl (including, for example, tetrahydroisoquinolin-4-yl or tetrahydroisoquinolin-6-yl, and the like), pyrrolo[3,2-c]pyridinyl (including, for example, pyrrolo[3,2-c]pyridin-2-yl or pyrrolo[3,2-c]pyridin-7-yl, and the like), pyrrolo[1,2-b]pyridazinyl, imidazo[1,2-a]pyridinyl, thiazolyl, benzo[d][1,3]dioxolyl, 2,3-dihydrobenzo[b][1,4]dioxinyl, furo[2,3-d]thiazolyl, thieno[2,3-d]oxazolyl, thieno[3,2-b]furanyl, furo[2,3-d]pyrimidinyl, furo[3,2-b]pyridinyl, furo[3,2-c]pyridinyl, 6,7-dihydro-5H-cyclopenta[b]pyridinyl, 1,4,5,6-tetrahydropyrrolo[3,4-c]pyrazolyl, 1,4,5,6-tetrahydropyrrolo[3,4-d]imidazolyl, and 7,8-dihydro-6H-cyclopenta[g]quinoxalinyl.

“Heteroaryloxy” means a group of the formula —OR, where R is heteroaryl. In certain embodiments heteroaryloxy includes pyridinyloxy.

“Heteroarylalkyloxy” means a group of the formula —O—R—R′, where R is alkylene and R′ is heteroaryl. In certain embodiments, heteroarylalkyloxy includes:

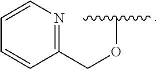 [SMILES: *OCc1ccccn1]

“Heterocycloalkyl” means a saturated or partially unsaturated (but not aromatic) monovalent monocyclic ring of 3 to 9 ring atoms, or a saturated or partially unsaturated (but not aromatic) monovalent bicyclic ring of 5 to 12 ring atoms in which one or more ring atoms is a heteroatom independently selected from —O—, —S(O)ₙ— (n is 0, 1, or 2), —N=(trivalent nitrogen), or —NH—, and the remaining ring atoms are carbon. In certain embodiments, the heterocycloalkyl group comprises one, two, three, or four ring heteroatoms, independently selected from —O—, —S(O)ₙ— (n is 0, 1, or 2), —N=(trivalent nitrogen), or —NH—, and the remaining ring atoms are carbon. Heterocycloalkyl groups include fused, bridged and spiro heterocycloalkyl bicyclic rings. For example, when fused, the heterocycloalkyl group may comprise two rings that share adjacent atoms (e.g., one covalent bond). When bridged, the heterocycloalkyl group may comprise two rings that share three or more atoms, separating the two bridgehead atoms by a bridge containing at least one atom. When spiro, the heterocycloalkyl group may comprise two rings that share only one single atom, the spiro atom, which may be, for example, a quaternary carbon. In certain embodiments, heterocycloalkyl is a saturated or partially unsaturated monocyclic group of 4 to 6 ring atoms, or a saturated or partially unsaturated bicyclic group of 7 to 8 ring atoms. In certain embodiments, heterocycloalkyl group contains 4 to 8 ring atoms. In certain embodiments, heterocycloalkyl group contains 8 ring atoms. When a heterocycloalkyl group contains from 4 to 6 ring atoms, it may be referred to herein as 4-6 membered heterocycloalkyl. When a heterocycloalkyl group contains from 7 to 8 ring atoms, it may be referred to herein as 7-8 membered heterocycloalkyl.

In certain embodiments, heterocycloalkyl includes, but is not limited to, azetidinyl, pyrrolidinyl, 2-oxopyrrolidinyl, 2,5-dihydro-1H-pyrrolinyl, 2,5-dioxo-1H-pyrrolyl, 2,5-dioxo-pyrrolidinyl, 2,5-dihydro-1H-pyrrolyl, piperidinyl, 2-oxopiperidinyl, 4-piperidonyl, morpholinyl, piperazinyl, 2-oxopiperazinyl, dioxopiperazinyl, pyranyl, tetrahydropyranyl, tetrahydrothiopyranyl, 1,3-dioxinyl, 1,3-dioxanyl, 1,4-dioxinyl, 1,4-dioxanyl, thiomorpholinyl, thiamorpholinyl, perhydroazepinyl, pyrazolidinyl, imidazolinyl, imidazolidinyl, 2,4-dioxo-imidazolidinyl, dihydropyridinyl, tetrahydropyridinyl, oxazolinyl, oxazolidinyl, isoxazolidinyl, thiazolinyl, thiazolidinyl, quinuclidinyl, isothiazolidinyl, octahydroindolyl, octahydroisoindolyl, octahydropyrrolo[3,4-c]pyrrolinyl, decahydroisoquinolyl, tetrahydrofuryl, 2-azaspiro[3.3]heptanyl, 4,7-diazaspiro[2.5]octane, 1,6-diazaspiro[3.3]heptanyl, 7-azabicyclo[2.2.1]heptanyl, and 8-azabicyclo[3.2.1]octanyl.

In certain embodiments, heterocycloalkyl includes, but is not limited to:

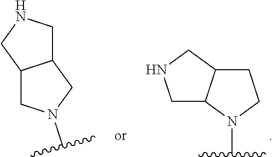 [SMILES: *N1CC2CNCC2C1 ; *N1CCC2CNCC21]

In certain embodiments, heterocycloalkyl includes, but is not limited to:

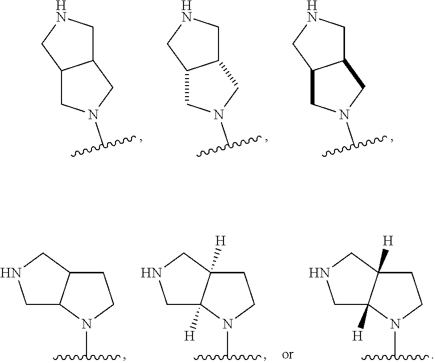

In certain embodiments, heterocycloalkyl includes, but is not limited to:

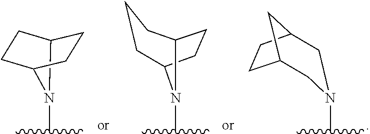 [SMILES: *N1C2CCC1CC2 ; *N1C2CCCC1CC2 ; *N1CC2CCC(C2)C1]

In certain embodiments, heterocycloalkyl includes, but is not limited to:

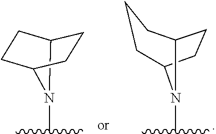 [SMILES: *N1C2CCC1CC2 ; *N1C2CCCC1CC2]

In certain embodiments, heterocycloalkyl includes, but is not limited to:

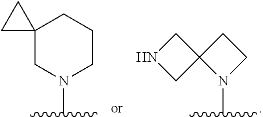 [SMILES: *N1CCCC2(CC2)C1 ; *N1CCC12CNC2]

“(Heterocyclolalkyl)alkyl” means an alkyl group, substituted with at least one heterocycloalkyl group. In certain embodiments, alkyl group is substituted with one or two heterocycloalkyl groups.

“Heterocycloalkyloxy” means a group of the formula —OR, where R is heterocycloalkyl group, as defined herein.

“(Heterocycloalkyl)alkyloxy” means a group of the formula —OR, where R is a (heterocycloalkyl)alkyl group, as defined herein.

“Spirocycloalkyl” means alkylene where both ends are attached to the same carbon atom to form a ring. For example, the same carbon may a quaternary carbon. In certain embodiments, spirocycloalkyl includes C₃-spirocycloalkyl (i.e., spirocyclopropyl), C₄-spirocycloalkyl, C₅-spirocycloalkyl, C₆-spirocycloalkyl, C₇-spirocycloalkyl, or C₈-spirocycloalkyl.

In some embodiments, compounds of the described herein exist as stereoisomers, wherein asymmetric or chiral centers are present. The term (R) and (S) used herein are configurations as defined in IUPAC 1974 Recommendations for Section E, Fundamental Stereochemistry, Pure Appl. Chem., (1976), 45:13-30, hereby incorporated by reference. The embodiments described herein specifically includes the various stereoisomers and mixtures thereof.

“Stereoisomers” include (but are not limited to) geometric isomers, enantiomers, diastereomers, and mixtures of geometric isomers, enantiomers or diastereomers. In some embodiments, individual stereoisomers of compounds are prepared synthetically from commercially available starting materials which contain asymmetric or chiral centers or by preparation of racemic mixtures followed by resolution. These methods of resolution are exemplified by (1) attachment of a mixture of enantiomers to a chiral auxiliary, separation of the resulting mixture of diastereomers by recrystallization or chromatography and liberation of the optically pure product from the auxiliary or (2) direct separation of the mixture of optical enantiomers on chiral chromatographic column.

“Amelioration” of the symptoms of a particular disorder by administration of a particular compound or pharmaceutical composition refers to any lessening of severity, delay in onset, slowing of progression, or shortening of duration, whether permanent or temporary, lasting or transient that can be attributed to or associated with administration of the compound or composition.

The terms “effective amount” or “therapeutically effective amount,” refer to a sufficient amount of an agent or a compound being administered which will relieve to some extent one or more of the symptoms of the disease or disorder being treated. The result includes reduction and/or alleviation of the signs, symptoms, or causes of a disease, or any other desired alteration of a biological system. For example, an “effective amount” for therapeutic uses is the amount of the composition comprising a compound as disclosed herein required to provide a clinically significant decrease in disease symptoms. An appropriate “effective” amount in any individual case is determined using any suitable technique, such as a dose escalation study.

“Excipient” or “pharmaceutically acceptable excipient” means a pharmaceutically-acceptable material, composition, or vehicle, such as a liquid or solid filler, diluent, solvent, or encapsulating material. Excipients include, for example, encapsulating materials or additives such as absorption accelerators, antioxidants, binders, buffers, coating agents, coloring agents, diluents, disintegrating agents, emulsifiers, extenders, fillers, flavoring agents, humectants, lubricants, perfumes, preservatives, propellants, releasing agents, sterilizing agents, sweeteners, solubilizers, wetting agents and mixtures thereof. In one embodiment, each component is “pharmaceutically acceptable” in the sense of being compatible with the other ingredients of a pharmaceutical formulation, and suitable for use in contact with the tissue or organ of humans and animals without excessive toxicity, irritation, allergic response, immunogenicity, or other problems or complications, commensurate with a reasonable benefit/risk ratio. See, e.g., Remington: The Science and Practice of Pharmacy, 21st ed.; Lippincott Williams & Wilkins: Philadelphia, Pa., 2005; Handbook of Pharmaceutical Excipients, 6th ed.; Rowe et al., Eds.; The Pharmaceutical Press and the American Pharmaceutical Association: 2009; Handbook of Pharmaceutical Additives, 3rd ed.; Ash and Ash Eds.; Gower Publishing Company: 2007; Pharmaceutical Preformulation and Formulation, 2nd ed.; Gibson Ed.; CRC Press LLC: Boca Raton, Fla., 2009.

“Pharmaceutically acceptable salt” refers to a formulation of a compound that does not cause significant irritation to an organism to which it is administered and does not abrogate the biological activity and properties of the compound. In certain instances, pharmaceutically acceptable salts are obtained by reacting a compound described herein, with acids such as hydrochloric acid, hydrobromic acid, sulfuric acid, nitric acid, phosphoric acid, methanesulfonic acid, ethanesulfonic acid, p-toluenesulfonic acid, salicylic acid and the like. In some instances, pharmaceutically acceptable salts are obtained by reacting a compound having acidic group described herein with a base to form a salt such as an ammonium salt, an alkali metal salt, such as a sodium or a potassium salt, an alkaline earth metal salt, such as a calcium or a magnesium salt, a salt of organic bases such as dicyclohexylamine, N-methyl-D-glucamine, tris(hydroxymethyl)methylamine, and salts with amino acids such as arginine, lysine, and the like, or by other methods previously determined. The pharmacologically acceptable salts not specifically limited as far as it can be used in medicaments. Examples of a salt that the compound forms with a base include the following: salts thereof with inorganic bases such as sodium, potassium, magnesium, calcium, and aluminum; salts thereof with organic bases such as methylamine, ethylamine and ethanolamine; salts thereof with basic amino acids such as lysine and ornithine; and ammonium salt. The salts may be acid addition salts, which are specifically exemplified by acid addition salts with the following: mineral acids such as hydrochloric acid, hydrobromic acid, hydroiodic acid, sulfuric acid, nitric acid, and phosphoric acid:organic acids such as formic acid, acetic acid, propionic acid, oxalic acid, malonic acid, succinic acid, fumaric acid, maleic acid, lactic acid, malic acid, tartaric acid, citric acid, methanesulfonic acid, and ethanesulfonic acid; acidic amino acids such as aspartic acid and glutamic acid.

The term “pharmaceutical composition” refers to a mixture of a compound described herein with other chemical components, such as an excipient. The pharmaceutical composition facilitates administration of the compound to an organism. Multiple techniques of administering a compound exist in the art including, but not limited to: intravenous, oral, aerosol, parenteral, ophthalmic, pulmonary and topical administration.

“Subject” refers to an animal, including, but not limited to, a primate (e.g., human), monkey, cow, pig, sheep, goat, horse, dog, cat, rabbit, rat, or mouse. The terms “subject” and “patient” are used interchangeably. In certain embodiments, the subject is a mammal. In certain embodiments, the subject is a human. In certain embodiments, the subject is an adult human. In certain embodiments, the subject is a human child.

“Treat,” “treating,” and “treatment,” in the context of treating a disease or disorder, are meant to include alleviating or abrogating a disorder, disease, or condition, or one or more of the symptoms associated with the disorder, disease, or condition; or to slowing the progression, spread or worsening of a disease, disorder or condition or of one or more symptoms thereof. Often, the beneficial effects that a subject derives from a therapeutic agent do not result in a complete cure of the disease, disorder or condition.

### EMBODIMENTS

The following paragraphs present a number of embodiments of the compounds disclosed herein. In each instance the embodiment includes both the recited compound(s) as well as a single stereoisomer or mixture of stereoisomers thereof, as well as a pharmaceutically acceptable salt thereof.

In one aspect, provided herein is a compound of Formula I:

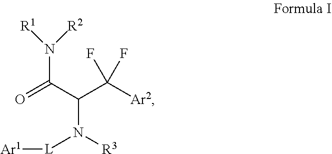

where

R¹ is alkyl, aminoalkyl, alkylaminoalkyl, or dialkylaminoalkyl;

R² is alkyl, aminoalkyl, alkylaminoalkyl, or dialkylaminoalkyl;

or R¹ and R² together with the nitrogen to which they are attached form a 4-8 membered heterocycloalkyl ring optionally substituted with 1, 2, or 3 R¹⁰ groups; or a bicyclic heteroaryl ring with 8 ring atoms, where 1-3 ring atoms are nitrogen;

R³ is H, alkyl, or haloalkyl;

L is S(O)_{q}, C₁₋₃alkylene-S(O)_{q}, C(O)—C(O), or C*H(OH)C(O), wherein the alkylene in the C₁₋₃alkylene-S(O) q forms a covalent bond with Ar¹, and wherein the “C*” in the C*H(OH)C(O) forms a covalent bond with Ar¹;

q is 0, 1, or 2;

Ar¹ is aryl or heteroaryl, each optionally substituted with 1 or 2 R¹¹ groups;

Ar² is aryl or heteroaryl, each optionally substituted with 1 or 2 R¹² groups;

each R¹⁰ is independently halo, cyano, nitro, amino, alkylamino, dialkylamino, aminoalkyl, alkylaminoalkyl, dialkylaminoalkyl, aminocarbonyl, alkylaminocarbonyl, dialkylaminocarbonyl, or C(═NOR^{10a})(NR^{10b}R^{10c});

R^{10a}, R^{10b}, and R^{10c} are each independently H or alkyl;

each R¹¹ is independently halo, cyano, nitro, alkyl, alkoxy, hydroxy, cycloalkyl, (cycloalkyl)alkyl, heterocycloalkyl, (heterocycloalkyl)alkyl, cycloalkyloxy, heterocycloalkyloxy, (cycloalkyl)alkyloxy, cycloalkyloxyalkyl, aryl, aryloxy, arylalkyloxy, heteroaryl, heteroaryloxy, or heteroarylalkyloxy; wherein each aryl and heteroaryl, whether alone or as part of another group, are each independently optionally substituted with halo, alkyl, haloalkyl, or alkoxy;

each R¹² is independently halo, cyano, nitro, hydroxy, alkyl, haloalkyl, haloalkenyl, alkoxy, cycloalkyloxy, cycloalkyl, heterocycloalkyl, cycloalkyl substituted with haloalkyl, (cycloalkyl)alkyloxy, heterocycloalkyloxy, (heterocycloalkyl)alkyloxy, aryl, heteroaryl, aryloxy, arylalkyloxy, heteroaryloxy, or heteroarylalkyloxy; wherein each aryl and heteroaryl, whether alone or as part of another group, are each independently optionally substituted with 1 or 2 groups selected from halo, alkyl, haloalkyl, and alkoxy; and

optionally a single stereoisomer or mixture of stereoisomers thereof and additionally optionally a pharmaceutically acceptable salt thereof.

In certain embodiments, provided herein is a compound of Formula I:

where

R¹ is alkyl or aminoalkyl;

R² is alkyl or aminoalkyl;

or R¹ and R² together with the nitrogen to which they are attached form a 4-8 membered heterocycloalkyl ring optionally substituted with 1, 2, or 3 R¹⁰ groups; or a bicyclic heteroaryl ring with 8 ring atoms, where 1-3 ring atoms are nitrogen;

R³ is H, alkyl, or haloalkyl;

L is S(O)₂, C₂alkylene-S(O)₂, C(O)—C(O), or C*H(OH)C(O), wherein the alkylene in the C₂alkylene-S(O) q forms a covalent bond with Ar¹, and wherein the “C*” in the C*H(OH)C(O) forms a covalent bond with Ar¹;

q is 2;

Ar¹ is aryl or heteroaryl, each optionally substituted with 1 or 2 R¹¹ groups;

Ar² is aryl or heteroaryl, each optionally substituted with 1 or 2 R¹² groups;

each R¹⁰ is independently halo, cyano, amino, dialkylamino, aminoalkyl, aminocarbonyl, or C(═NOH)(NH₂);

each R¹¹ is independently halo, alkyl, alkoxy, (cycloalkyl)alkyl, cycloalkyloxy, (cycloalkyl)alkyloxy, cycloalkyloxyalkyl, aryl, aryloxy, arylalkyloxy, or heteroaryloxy; wherein each aryl and heteroaryl, whether alone or as part of another group, are each independently optionally substituted with halo;

each R¹² is independently halo, hydroxy, haloalkyl, haloalkenyl, alkoxy, cycloalkyloxy, cycloalkyl substituted with haloalkyl, (heterocycloalkyl)alkyloxy, aryl, heteroaryl, aryloxy, or arylalkyloxy; wherein each aryl and heteroaryl, whether alone or as part of another group, are each independently optionally substituted with 1 or 2 groups selected from halo and haloalkyl; or

optionally a single stereoisomer or mixture of stereoisomers thereof and additionally optionally a pharmaceutically acceptable salt thereof.

In certain embodiments, the compound of Formula (I) is according to Formula I(a):

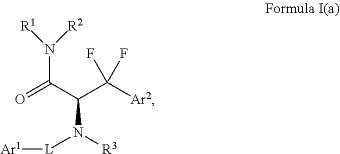

and optionally a single stereoisomer or mixture of stereoisomers thereof and additionally optionally a pharmaceutically acceptable salt thereof.

In certain embodiments, the compound of Formula (I) is according to Formula I(b):

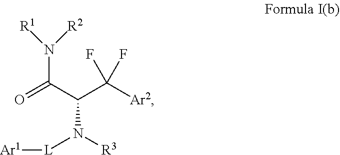

and optionally a single stereoisomer or mixture of stereoisomers thereof and additionally optionally a pharmaceutically acceptable salt thereof.

In certain embodiments, L is S(O)_{q} and q is 0, 1, or 2. In certain embodiments, L is S(O)_{q} and q is 2. In certain embodiments, L is C₁₋₃alkylene-S(O)_{q}, wherein the alkylene in the C₁₋₃alkylene-S(O) q forms a covalent bond with Ar¹. In certain embodiments, L is C₁₋₃alkylene-S(O)_{q} and q is 2. In certain embodiments, L is C₁alkylene-S(O)₂. In certain embodiments, L is C₂alkylene-S(O)₂. In certain embodiments, L is C₃alkylene-S(O)₂. In certain embodiments, L is C(O)—C(O). In certain embodiments, L is C*H(OH)C(O) wherein “C*” forms a covalent bond with Ar¹. In certain embodiments, L is S(O)₂ or C(O)—C(O). In certain embodiments, L is S(O)₂ or C*H(OH)C(O) wherein “C*” forms a covalent bond with Ar¹. In certain embodiments, L is C(O)—C(O) or C*H(OH)C(O) wherein “C*” forms a covalent bond with Ar¹.

In certain embodiments, the compound of Formula (I) is according to Formula I(c), Formula I(d), Formula I(e), or Formula I(f):

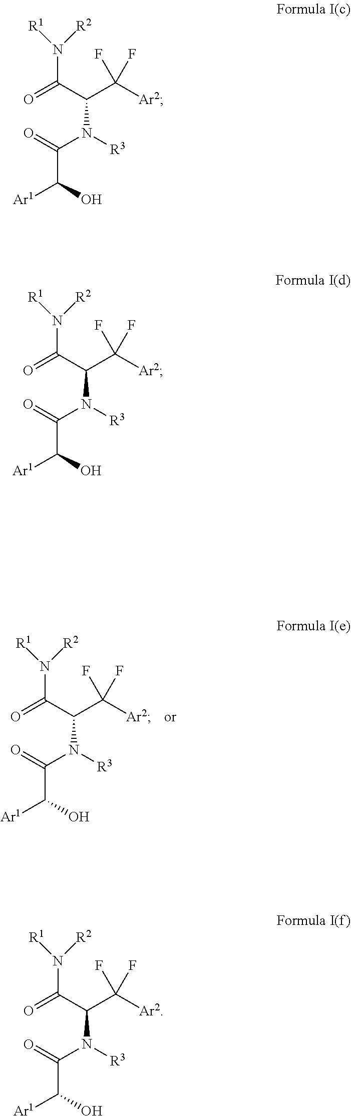

In certain embodiments, R¹ and R² are each independently alkyl, aminoalkyl, alkylaminoalkyl, or dialkylaminoalkyl. In certain embodiments, R¹ and R² are each alkyl. In certain embodiments, R¹ and R² are each methyl. In certain embodiments, R¹ and R² are each aminoalkyl. In certain embodiments, R¹ and R² are each aminopropyl. In certain embodiments, R¹ and R² are each independently alkyl or aminoalkyl.

In certain embodiments, R¹ and R² together with the nitrogen to which they are attached form a 4-8 membered heterocycloalkyl ring which is optionally substituted with 1, 2, or 3 R¹⁰ groups.

In certain embodiments, R¹ and R² together with the nitrogen to which they are attached form a 4-6 membered monocyclic heterocycloalkyl ring which is optionally substituted with 1, 2, or 3 R¹⁰ groups.

In certain embodiments, R¹ and R² together with the nitrogen to which they are attached form a 4 membered monocyclic heterocycloalkyl ring which is optionally substituted with 1, 2, or 3 R¹⁰ groups. In certain embodiments, R¹ and R² together with the nitrogen to which they are attached form a 5 membered monocyclic heterocycloalkyl ring which is optionally substituted with 1, 2, or 3 R¹⁰ groups. In certain embodiments, R¹ and R² together with the nitrogen to which they are attached form a 6 membered monocyclic heterocycloalkyl ring which is optionally substituted with 1, 2, or 3 R¹⁰ groups. In certain embodiments, R¹ and R² together with the nitrogen to which they are attached form a 5-6 membered monocyclic heterocycloalkyl ring which is optionally substituted with 1, 2, or 3 R¹⁰ groups.

In certain embodiments, R¹ and R² together with the nitrogen to which they are attached form a 7-8 membered bicyclic heterocycloalkyl ring which is optionally substituted with 1, 2, or 3 R¹⁰ groups.

In certain embodiments, R¹ and R² together with the nitrogen to which they are attached form a 7 membered bicyclic heterocycloalkyl ring which is optionally substituted with 1, 2, or 3 R¹⁰ groups. In certain embodiments, R¹ and R² together with the nitrogen to which they are attached form a 8 membered bicyclic heterocycloalkyl ring which is optionally substituted with 1, 2, or 3 R¹⁰ groups.

In certain embodiments, R¹ and R² together with the nitrogen to which they are attached form a 4-6 membered monocyclic heterocycloalkyl ring or a 7-8 membered bicyclic heterocycloalkyl ring which is optionally substituted with 1, 2, or 3 R¹⁰ groups.

In certain embodiments, R¹ and R² together with the nitrogen to which they are attached form a 8 membered heterocycloalkyl ring which is optionally substituted with 1, 2, or 3 R¹⁰ groups.

In certain embodiments, the heterocycloalkyl ring has one of the following

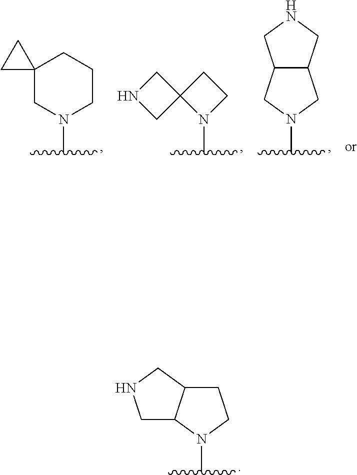

In certain embodiments, the heterocycloalkyl ring has one of the following structures:

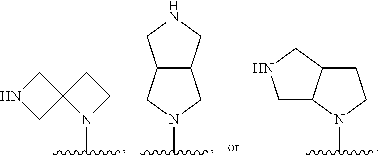

In certain embodiments, the heterocycloalkyl ring has one of the following structures:

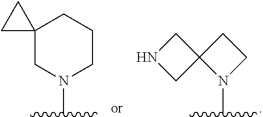 [SMILES: *N1CCCC2(CC2)C1 ; *N1CCC12CNC2]

In certain embodiments, R¹ and R² together with the nitrogen to which they are attached form a bicyclic heteroaryl ring with 8 ring atoms, where 1-3 ring atoms are nitrogen.

In certain embodiments, the bicyclic heteroaryl ring has one of the following structures:

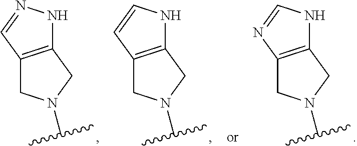 [SMILES: *N1Cc2cn[nH]c2C1 ; *N1Cc2cc[nH]c2C1 ; *N1Cc2nc[nH]c2C1]

In certain embodiments, the bicyclic heteroaryl ring has the following structure:

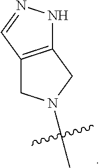 [SMILES: *N1Cc2cn[nH]c2C1]

In certain embodiments, the bicyclic heteroaryl ring has the following structure:

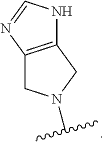 [SMILES: *N1Cc2nc[nH]c2C1]

In certain embodiments, the compound of Formula (I) is according to Formula II(a), Formula II(b), Formula II(c), Formula II(d), Formula II(e), Formula II(f), Formula II(g), or Formula II(h):

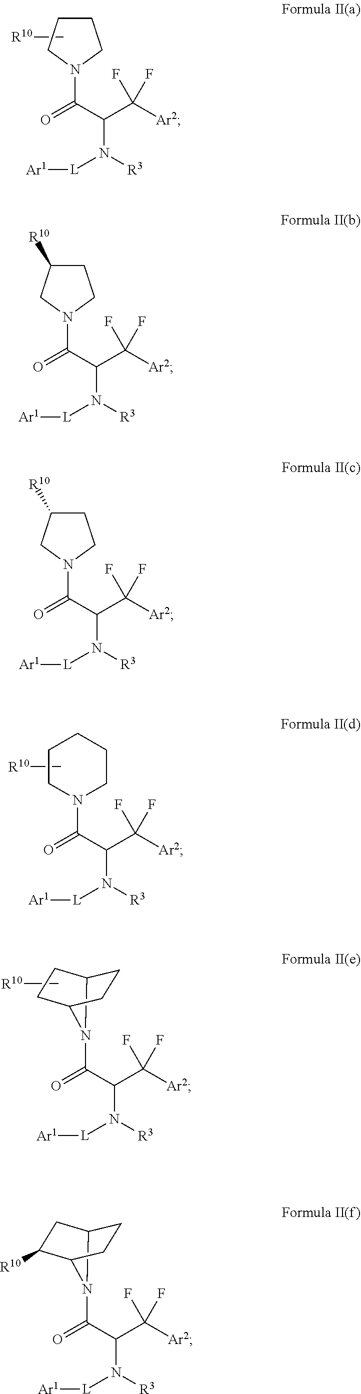

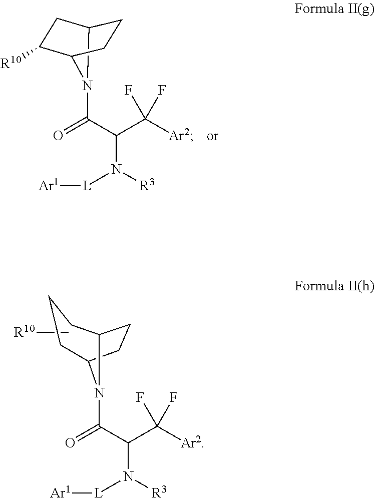

In certain embodiments, the compound of Formula (I) is according to Formula III(a), or Formula III(b):

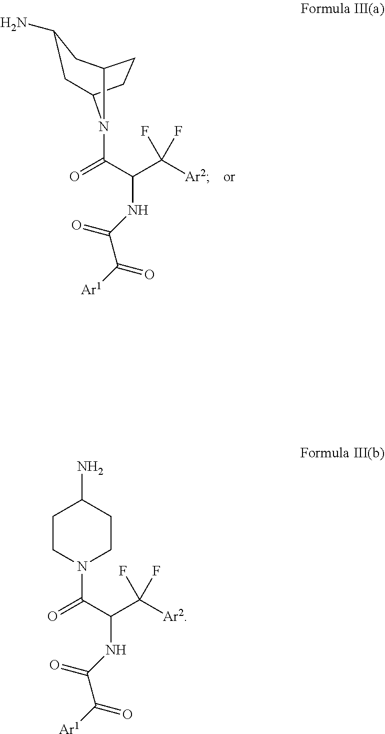

In certain embodiments, each R¹⁰ is independently halo, cyano, amino, alkylamino, dialkylamino, aminoalkyl, alkylaminoalkyl, dialkylaminoalkyl, aminocarbonyl, alkylaminocarbonyl, dialkylaminocarbonyl, or C(═NOH)(NH₂). In certain embodiments, each R¹⁰ is independently halo, amino, alkylamino, or dialkylamino. In certain embodiments, each R¹⁰ is independently fluoro, amino, methylamino, or dimethylamino. In certain embodiments, each R¹⁰ is independently fluoro or amino. In certain embodiments, each R¹⁰ is independently amino, alkylamino, or dialkylamino.

In certain embodiments, each R¹⁰ is independently halo, cyano, amino, dialkylamino, aminoalkyl, aminocarbonyl, or C(═NOH)(NH₂).

In certain embodiments, R¹⁰ is amino. In certain embodiments, R¹⁰ is dimethylamino. In certain embodiments, R¹⁰ is fluoro or chloro. In certain embodiments, R¹⁰ is fluoro. In certain embodiments, R¹⁰ is cyano. In certain embodiments, R¹⁰ is aminocarbonyl. In certain embodiments, R¹⁰ is aminoalkyl, alkylaminoalkyl, or dialkylaminoalkyl. In certain embodiments, R¹⁰ is aminomethyl. In certain embodiments, R¹⁰ is C(═NOH)(NH₂).

In certain embodiments, R¹ and R² together with the nitrogen to which they are attached form a 6-8 membered heterocycloalkyl ring substituted with an amino, alkylamino, or dialkylamino group. In certain embodiments, R¹ and R² together with the nitrogen to which they are attached form a 6-8 membered heterocycloalkyl ring substituted with an amino group. In certain embodiments, R¹ and R² together with the nitrogen to which they are attached form a 6-8 membered heterocycloalkyl ring substituted with a dialkylamino group. In certain embodiments, R¹ and R² together with the nitrogen to which they are attached form a 6-8 membered heterocycloalkyl ring substituted with a aminoalkyl group. In certain embodiments, R¹ and R² together with the nitrogen to which they are attached form a 6-8 membered heterocycloalkyl ring substituted with a aminocarbonyl group. In certain embodiments, R¹ and R² together with the nitrogen to which they are attached form a 6-8 membered heterocycloalkyl ring substituted with a cyano group. In certain embodiments, R¹ and R² together with the nitrogen to which they are attached form a 6-8 membered heterocycloalkyl ring substituted with an amino group and a halo group.

In certain embodiments, the compound of Formula (I) is according to Formula IV(a), Formula IV(b), Formula IV(c), or Formula IV(d):

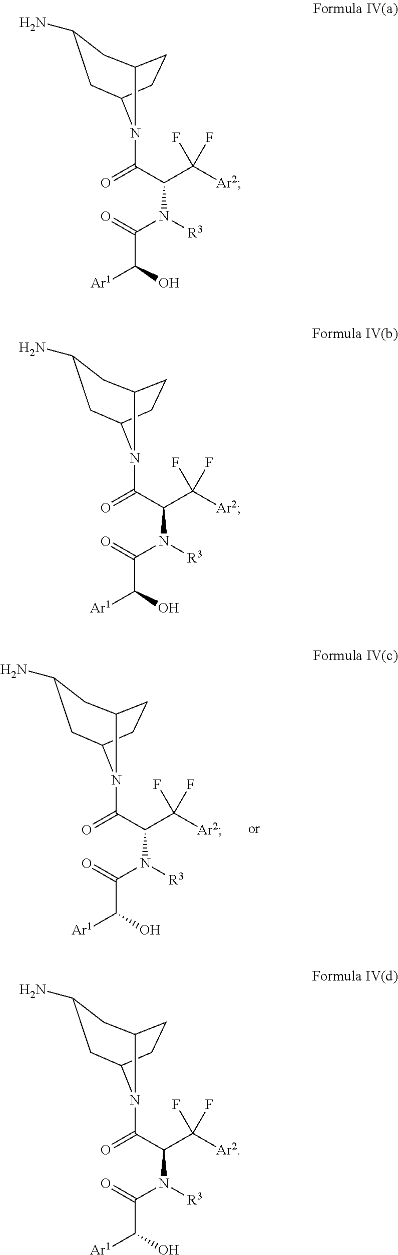

In certain embodiments, the compound of Formula (I) is according to Formula V(a), Formula V(b), Formula V(c), or Formula V(d):

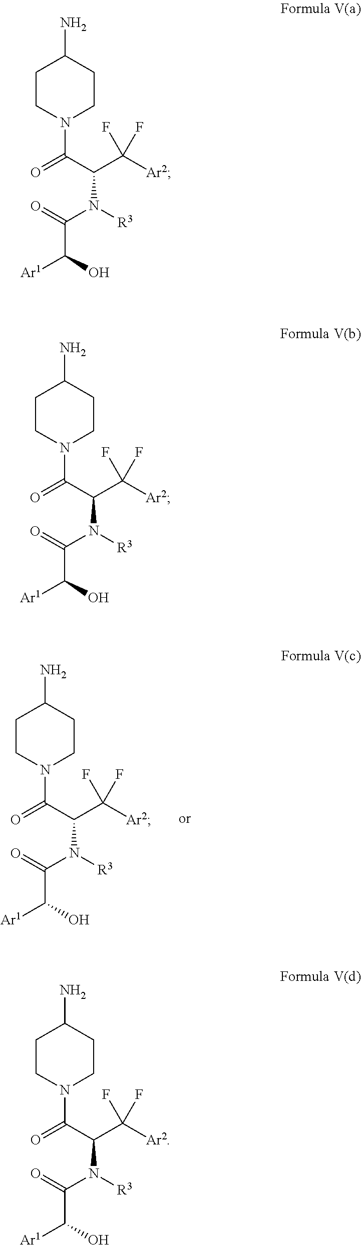

In certain embodiments, R³ is H, alkyl, or haloalkyl. In certain embodiments, R³ is H. In certain embodiments, R³ is alkyl. In certain embodiments, R³ is methyl, ethyl, or, i-propyl. In certain embodiments, R³ is methyl. In certain embodiments, R³ is ethyl. In certain embodiments, R³ is i-propyl. In certain embodiments, R³ is haloalkyl. In certain embodiments, R³ is trifluoromethyl, 1,1,1-trifluoroethyl, or pentafluoroethyl. In certain embodiments, R³ is 1,1,1-trifluoroethyl.

In certain embodiments, Ar¹ is optionally substituted aryl or optionally substituted heteroaryl. In certain embodiments, Ar¹ is unsubstituted aryl or unsubstituted heteroaryl. In certain embodiments, Ar¹ is substituted aryl or unsubstituted heteroaryl. In certain embodiments, Ar¹ is substituted aryl or substituted heteroaryl. In certain embodiments, Ar¹ is unsubstituted aryl or substituted heteroaryl.

In certain embodiments, the Ar¹ group is unsubstituted. In certain embodiments, the Ar¹ group is substituted with 1 or 2 R¹¹ groups. In certain embodiments, the Ar¹ group is substituted with 1 R¹¹ group. In certain embodiments, the Ar¹ group is substituted with 2 R¹¹ groups.

In certain embodiments, Ar¹ is aryl optionally substituted with 1 or 2 R¹¹ groups. In certain embodiments, Ar¹ is aryl optionally substituted with 1 R¹¹ group. In certain embodiments, Ar¹ is aryl optionally substituted with 2 R¹¹ groups. In certain embodiments, Ar¹ is unsubstituted aryl. In certain embodiments, Ar¹ is aryl substituted with 1 or 2 R¹¹ groups. In certain embodiments, Ar¹ is aryl substituted with 1 R¹¹ group. In certain embodiments, Ar¹ is aryl substituted with 2 R¹¹ groups. In certain embodiments, Ar¹ is phenyl or naphthyl, wherein each is optionally substituted with 1 or 2 R¹¹ groups. In certain embodiments, Ar¹ is phenyl or naphthyl, wherein each is optionally substituted with 1 R¹¹ group. In certain embodiments, Ar¹ is phenyl or naphthyl, wherein each is optionally substituted with 2 R¹¹ groups. In certain embodiments, unsubstituted phenyl or unsubstituted naphthyl. In certain embodiments, Ar¹ is phenyl or naphthyl, wherein each is substituted with 1 or 2 R¹¹ groups. In certain embodiments, Ar¹ is phenyl or naphthyl, wherein each is substituted with 1 R¹¹ group. In certain embodiments, Ar¹ is phenyl or naphthyl, wherein each is substituted with 2 R¹¹ groups. In certain embodiments, Ar¹ is phenyl optionally substituted with 1 or 2 R¹¹ groups. In certain embodiments, Ar¹ is phenyl optionally substituted with 1 R¹¹ group. In certain embodiments, Ar¹ is phenyl optionally substituted with 2 R¹¹ groups. In certain embodiments, Ar¹ is unsubstituted phenyl. In certain embodiments, Ar¹ is phenyl substituted with 1 or 2 R¹¹ groups. In certain embodiments, Ar¹ is phenyl substituted with 1 R¹¹ group. In certain embodiments, Ar¹ is phenyl substituted with 2 R¹¹ groups. In certain embodiments, Ar¹ is naphthyl optionally substituted with 1 or 2 R¹¹ groups. In certain embodiments, Ar¹ is naphthyl optionally substituted with 1 R¹¹ group. In certain embodiments, Ar¹ is naphthyl optionally substituted with 2 R¹¹ groups. In certain embodiments, Ar¹ is unsubstituted naphthyl. In certain embodiments, Ar¹ is naphthyl substituted with 1 or 2 R¹¹ groups. In certain embodiments, Ar¹ is naphthyl substituted with 1 R¹¹ group. In certain embodiments, Ar¹ is naphthyl substituted with 2 R¹¹ groups.

In certain embodiments, Ar¹ is heteroaryl optionally substituted with 1 or 2 R¹¹ groups. In certain embodiments, Ar¹ is heteroaryl optionally substituted with 1 R¹¹ group. In certain embodiments, Ar¹ is heteroaryl optionally substituted with 2 R¹¹ groups. In certain embodiments, Ar¹ is unsubstituted heteroaryl. In certain embodiments, Ar¹ is heteroaryl substituted with 1 or 2 R¹¹ groups. In certain embodiments, Ar¹ is heteroaryl substituted with 1 R¹¹ group. In certain embodiments, Ar¹ is heteroaryl substituted with 2 R¹¹ groups. In certain embodiments, Ar¹ is a 5-6 membered heteroaryl or a 9-10 membered bicyclic heteroaryl, wherein each is optionally substituted with 1 or 2 R¹¹ groups. In certain embodiments, Ar¹ is a 5-6 membered heteroaryl or a 9-10 membered bicyclic heteroaryl, wherein each is optionally substituted with 1 R¹¹ group. In certain embodiments, Ar¹ is a 5-6 membered heteroaryl or a 9-10 membered bicyclic heteroaryl, wherein each is optionally substituted with 2 R¹¹ groups. In certain embodiments, Ar¹ is an unsubstituted 5-6 membered heteroaryl or an unsubstituted 9-10 membered bicyclic heteroaryl. In certain embodiments, Ar¹ is a 5-6 membered heteroaryl or a 9-10 membered bicyclic heteroaryl, wherein each is substituted with 1 or 2 R¹¹ groups. In certain embodiments, Ar¹ is a 5-6 membered heteroaryl or a 9-10 membered bicyclic heteroaryl, wherein each is substituted with 1 R¹¹ group. In certain embodiments, Ar¹ is a 5-6 membered heteroaryl or a 9-10 membered bicyclic heteroaryl, wherein each is substituted with 2 R¹¹ groups. In certain embodiments, Ar¹ is a 5-6 membered heteroaryl or a 9-10 membered bicyclic heteroaryl, each containing 1 or 2 ring heteroatoms, wherein each is optionally substituted with 1 or 2 R¹¹ groups. In certain embodiments, Ar¹ is a 5-6 membered heteroaryl or a 9-10 membered bicyclic heteroaryl, each containing 1 or 2 ring heteroatoms, wherein each is optionally substituted with 1 R¹¹ groups. In certain embodiments, Ar¹ is a 5-6 membered heteroaryl or a 9-10 membered bicyclic heteroaryl, each containing 1 or 2 ring heteroatoms, wherein each is optionally substituted with 2 R¹¹ groups. In certain embodiments, Ar¹ is an unsubstituted 5-6 membered heteroaryl or an unsubstituted 9-10 membered bicyclic heteroaryl, each containing 1 or 2 ring heteroatoms. In certain embodiments, Ar¹ is a 5-6 membered heteroaryl or a 9-10 membered bicyclic heteroaryl, each containing 1 or 2 ring heteroatoms, wherein each is substituted with 1 or 2 R¹¹ groups. In certain embodiments, Ar¹ is a 5-6 membered heteroaryl or a 9-10 membered bicyclic heteroaryl, each containing 1 or 2 ring heteroatoms, wherein each is substituted with 1 R¹¹ group. In certain embodiments, Ar¹ is a 5-6 membered heteroaryl or a 9-10 membered bicyclic heteroaryl, each containing 1 or 2 ring heteroatoms, wherein each is substituted with 2 R¹¹ groups. In certain embodiments, Ar¹ is a 5-6 membered heteroaryl or a 9-10 membered bicyclic heteroaryl, each containing 1 ring heteroatom, wherein each is optionally substituted with 1 or 2 R¹¹ groups. In certain embodiments, Ar¹ is a 5-6 membered heteroaryl containing 1 ring heteroatom or a 9-10 membered bicyclic heteroaryl containing 1 or 2 ring heteroatoms, wherein each is optionally substituted with 1 or 2 R¹¹ groups. In certain embodiments, Ar¹ is a 5-6 membered heteroaryl containing 1 ring heteroatom or a 9-10 membered bicyclic heteroaryl containing 2 ring heteroatoms, wherein each is optionally substituted with 1 or 2 R¹¹ groups. In certain embodiments, Ar¹ is a 5-6 membered heteroaryl containing 1 or 2 ring heteroatoms, wherein the heteroaryl is optionally substituted with 1 or 2 R¹¹ groups. In certain embodiments, Ar¹ is a 5-6 membered heteroaryl containing 1 ring heteroatom, wherein the heteroaryl is optionally substituted with 1 or 2 R¹¹ groups. In certain embodiments, Ar¹ is a 5-6 membered heteroaryl containing 2 ring heteroatoms, wherein the heteroaryl is optionally substituted with 1 or 2 R¹¹ groups. In certain embodiments, Ar¹ is a 9-10 membered bicyclic heteroaryl containing 1 or 2 ring heteroatoms, wherein the heteroaryl is optionally substituted with 1 or 2 R¹¹ groups. In certain embodiments, Ar¹ is a 9-10 membered bicyclic heteroaryl containing 1 ring heteroatom, wherein the heteroaryl is optionally substituted with 1 or 2 R¹¹ groups. In certain embodiments, Ar¹ is a 9-10 membered bicyclic heteroaryl containing 2 ring heteroatoms, wherein the heteroaryl is optionally substituted with 1 or 2 R¹¹ groups. In certain embodiments, the heteroaryl contains 1 or 2 ring nitrogen atoms or a ring sulfur atom. In certain embodiments, the heteroaryl contains a ring nitrogen atom and a ring sulfur atom. In certain embodiments, the heteroaryl contains 1 or 2 ring nitrogen atoms. In certain embodiments, the heteroaryl contains a ring sulfur atom. In certain embodiments, the heteroaryl contains 1 ring nitrogen atom. In certain embodiments, the heteroaryl contains 2 ring nitrogen atoms.

In certain embodiments, Ar¹ is 2-pyridyl, 3-pyridyl, indazolyl, thienyl, or benzothienyl, wherein each is optionally substituted with 1 or 2 R¹¹ groups. In certain embodiments, Ar¹ is unsubstituted 2-pyridyl, unsubstituted 3-pyridyl, unsubstituted indazolyl, unsubstituted thienyl, or unsubstituted benzothienyl. In certain embodiments, Ar¹ is 2-pyridyl, 3-pyridyl, indazolyl, thienyl, or benzothienyl, wherein each is substituted with 1 R¹¹ group. In certain embodiments, Ar¹ is 2-pyridyl, 3-pyridyl, indazolyl, thienyl, or benzothienyl, wherein each is substituted with 2 R¹¹ groups. In certain embodiments, Ar¹ is 2-pyridyl, 3-pyridyl, or indazolyl, wherein each is optionally substituted with 1 or 2 R¹¹ groups. In certain embodiments, Ar¹ is thienyl or benzothienyl, wherein each is optionally substituted with 1 or 2 R¹¹ groups.

In certain embodiments, each R¹¹ is independently halo, alkyl, alkoxy, hydroxy, cycloalkyl, (cycloalkyl)alkyl, heterocycloalkyl, (heterocycloalkyl)alkyl, cycloalkyloxy, heterocycloalkyloxy, (cycloalkyl)alkyloxy, cycloalkyloxyalkyl, aryl, aryloxy, arylalkyloxy, heteroaryl, heteroaryloxy, or heteroarylalkyloxy; wherein each aryl and heteroaryl, whether alone or as part of another group, are each independently optionally substituted with halo, alkyl, haloalkyl, or alkoxy. In certain embodiments, each R¹¹ is independently halo, alkyl, alkoxy, or hydroxy.

In certain embodiments, each R¹¹ is independently cycloalkyl, (cycloalkyl)alkyl, heterocycloalkyl, (heterocycloalkyl)alkyl, cycloalkyloxy, heterocycloalkyloxy, (cycloalkyl)alkyloxy, cycloalkyloxyalkyl, aryl, aryloxy, arylalkyloxy, heteroaryl, heteroaryloxy, or heteroarylalkyloxy; wherein each aryl and heteroaryl, whether alone or as part of another group, are each independently optionally substituted with halo, alkyl, haloalkyl, or alkoxy.

In certain embodiments, each R¹¹ is independently alkoxy, cycloalkyloxy, heterocycloalkyloxy, (cycloalkyl)alkyloxy, cycloalkyloxyalkyl, aryloxy, arylalkyloxy, heteroaryloxy, or heteroarylalkyloxy; wherein each aryl and heteroaryl, whether alone or as part of another group, are each independently optionally substituted with halo, alkyl, haloalkyl, or alkoxy.

In certain embodiments, each R¹¹ is independently halo, alkyl, alkoxy, (cycloalkyl)alkyl, cycloalkyloxy, (cycloalkyl)alkyloxy, cycloalkyloxyalkyl, aryl, aryloxy, arylalkyloxy, or heteroaryloxy; wherein each aryl and heteroaryl, whether alone or as part of another group, are each independently optionally substituted with halo. In certain embodiments, each R¹¹ is independently methyl, ethyl, n-propyl, i-propyl, cyclopentylmethyl, cyclopentylethyl, cyclohexylmethyl, cyclohexylethyl, methoxy, ethoxy, propoxy, i-propoxy, butoxy, i-butoxy, sec-butoxy, 2-ethylbutoxy, hexyloxy, cyclopropyloxy, cyclobutyloxy, cyclopentyloxy, cyclopentylmethyloxy, cyclopentylethyloxy, cyclopentyloxymethyl, cyclohexyloxy, cyclohexylmethyloxy, cyclohexylethyloxy, cyclohexyloxymethyl, fluorophenyl, chlorophenyl, benzyloxy, chlorobenzyloxy, fluorobenzyloxy, pyridyloxy, fluoropyridyloxy, or chloropyridyloxy. In certain embodiments, each R¹¹ is independently i-propyl, cyclopentylmethyl, cyclopentylethyl, i-propoxy, butoxy, i-butoxy, sec-butoxy, 2-ethylbutoxy, hexyloxy, cyclopropyloxy, cyclobutyloxy, cyclopentyloxy, cyclohexylmethyloxy, cyclohexylethyloxy, cyclohexyloxymethyl, 4-fluorophenyl, benzyloxy, chlorobenzyloxy, fluorobenzyloxy, pyridyloxy, or chloropyridyloxy.

In certain embodiments, each R¹¹ is independently fluoro, chloro, bromo, iodo, methyl, ethyl, n-propyl, i-propyl, methoxy, ethoxy, propoxy, i-propoxy, butoxy, i-butoxy, sec-butoxy, cyclopropyloxy, cyclobutyloxy, cyclopentyloxy, cyclohexyloxy, pyridyloxy, or benzyloxy.

In certain embodiments, each R¹¹ is independently methoxy, ethoxy, propoxy, i-butoxy, 2-ethylbutoxy, butoxy, hexyloxy, cyclopentylmethyloxy, cyclohexylmethyloxy, cyclohexylethyloxy, cyclopentyloxyethyl, 4-fluorophenyl, 4-fluorophenoxy, benzyloxy, 3-fluorobenzyloxy, 4-fluorobenzyloxy, 4-fluoro-2-pyridyloxy, or 4-chloro-2-pyridyloxy.

In certain embodiments, each R¹¹ is independently halo, cyano, nitro, alkyl, alkoxy, or hydroxy.

In certain embodiments, each R¹¹ is independently halo or alkoxy.

In certain embodiments, each R¹¹ is independently fluoro, chloro, bromo, iodo, methoxy, ethoxy, propoxy, i-propoxy, butoxy, i-butoxy, or sec-butoxy.

In certain embodiments, R¹¹ is (cycloalkyl)alkyloxy. In certain embodiments, R¹¹ is cyclopentylmethyloxy, cyclohexylmethyloxy, or cyclohexylethyloxy. In certain embodiments, R¹¹ is cyclohexylmethyloxy.

In certain embodiments, each R¹¹ is independently bromo or i-propoxy.

In certain embodiments, each R¹¹ is independently methyl, ethyl, n-propyl, i-propyl, methoxy, ethoxy, propoxy, i-propoxy, butoxy, i-butoxy, sec-butoxy, cyclopropyloxy, cyclobutyloxy, cyclopentyloxy, cyclohexyloxy, pyridyloxy, or benzyloxy.

In certain embodiments, R¹¹ is alkyl. In certain embodiments, R¹¹ is methyl, ethyl, n-propyl, or i-propyl. In certain embodiments, R¹¹ is i-propyl.

In certain embodiments, R¹¹ is alkoxy. In certain embodiments, R¹¹ is i-propoxy, butoxy, i-butoxy, or sec-butoxy. In certain embodiments, R¹¹ is i-propoxy. In certain embodiments, R¹¹ is butoxy. In certain embodiments, R¹¹ is i-butoxy. In certain embodiments, R¹¹ is sec-butoxy.

In certain embodiments, R¹¹ is i-propoxy, cyclopentyloxy, fluorophenyl, or cyclohexylmethoxy.

In certain embodiments, R¹¹ is cycloalkyloxy. In certain embodiments, R¹¹ is cyclopropyloxy, cyclobutyloxy, cyclopentyloxy, or cyclohexyloxy. In certain embodiments, R¹¹ is cyclopropyloxy. In certain embodiments, R¹¹ is cyclobutyloxy. In certain embodiments, R¹¹ is cyclopentyloxy. In certain embodiments, R¹¹ is cyclohexyloxy.

In certain embodiments, R¹¹ is arylalkyloxy. In certain embodiments, R¹¹ is benzyloxy.

In certain embodiments, R¹¹ is heteroaryloxy. In certain embodiments, R¹¹ is pyridyloxy. In certain embodiments, R¹¹ is 2-pyridyloxy.

In certain embodiments, R¹¹ is 4-fluorophenyl, cyclopentylmethyl, cyclopentyloxymethyl, or cylclopentyloxy.

In certain embodiments, R¹¹ is i-propoxy, cyclopentyloxy, fluorophenyl, or cyclohexylmethoxy.

In certain embodiments of R¹¹, where each aryl and heteroaryl, whether alone or as part of another group, are each independently optionally substituted with halo, alkyl, haloalkyl, or alkoxy, such embodiments include, for example, aryl, aryloxy, arylalkyloxy, heteroaryl, heteroaryloxy, and heteroarylalkyloxy optionally substituted with halo, alkyl, haloalkyl, or alkoxy.

In certain embodiments, Ar¹ is phenyl substituted with 1 or 2 R¹¹ groups; wherein each R¹¹ is independently alkoxy, (cycloalkyl)alkyloxy, cycloalkyloxyalkyl, aryl, aryloxy, arylalkyloxy, or heteroaryloxy, wherein each aryl and heteroaryl, whether alone or as part of another group, are each independently optionally substituted with halo. In certain embodiments, Ar¹ is phenyl substituted with 1 R¹¹ group selected from alkoxy, (cycloalkyl)alkyloxy, cycloalkyloxyalkyl, aryl, aryloxy, arylalkyloxy, and heteroaryloxy, wherein each aryl and heteroaryl of R¹¹, whether alone or as part of another group, are each independently optionally substituted with halo. In certain embodiments, Ar¹ is phenyl substituted with 1 R¹¹ group selected from alkoxy, (cycloalkyl)alkyloxy, cycloalkyloxyalkyl, aryl, aryloxy, arylalkyloxy, and heteroaryloxy, wherein each aryl and heteroaryl of R¹¹, whether alone or as part of another group, are each independently optionally substituted with halo, wherein halo is selected from fluoro and chloro. In certain embodiments, Ar¹ is phenyl substituted with 1 R¹¹ group selected from fluoro, chloro, bromo, and iodo. In certain embodiments, Ar¹ is phenyl substituted with 1 R¹¹ group selected from fluoro and chloro. In certain embodiments, Ar¹ is phenyl substituted with 1 R¹¹ group selected from i-butoxy, 2-ethylbutoxy, butoxy, hexyloxy, cyclopentylmethyloxy, cyclohexylmethyloxy, cyclohexylethyloxy, cyclopentyloxyethyl, 4-fluorophenyl, 4-fluorophenoxy, benzyloxy, 3-fluorobenzyloxy, 4-fluorobenzyloxy, 4-fluoro-2-pyridyloxy, and 4-chloro-2-pyridyloxy.

In certain embodiments, Ar¹ is naphthyl substituted with 1 or 2 R¹¹ groups, wherein each R¹¹ is independently halo, alkyl, alkoxy, cycloalkyloxy, arylalkyloxy, or heteroaryloxy. In certain embodiments, Ar¹ is naphthyl substituted with 1 R¹¹ group selected from halo, alkyl, alkoxy, cycloalkyloxy, arylalkyloxy, or heteroaryloxy. In certain embodiments, Ar¹ is phenyl substituted with 1 R¹¹ group selected from i-butoxy, 2-ethylbutoxy, butoxy, hexyloxy, cyclopentylmethyloxy, cyclohexylmethyloxy, cyclohexylethyloxy, cyclopentyloxyethyl, 4-fluorophenyl, 4-fluorophenoxy, benzyloxy, 3-fluorobenzyloxy, 4-fluorobenzyloxy, 4-fluoro-2-pyridyloxy, and 4-chloro-2-pyridyloxy.

In certain embodiments, Ar¹ is naphthyl substituted with 1 or 2 R¹¹ groups, wherein each R¹¹ is independently alkyl, alkoxy, cycloalkyloxy, arylalkyloxy, or heteroaryloxy. In certain embodiments, Ar¹ is naphthyl substituted with 1 R¹¹ group selected from alkyl, alkoxy, cycloalkyloxy, arylalkyloxy, and heteroaryloxy. In certain embodiments, Ar¹ is 2-pyridyl, 3-pyridyl, indazolyl, thienyl, or benzothienyl, wherein each Ar¹ is independently substituted with 1 R¹¹ group selected from (cycloalkyl)alkyl, cycloalkyloxy, cycloalkyloxyalkyl, and aryl optionally substituted with halo. In certain embodiments, Ar¹ is 2-pyridyl, 3-pyridyl, indazolyl, thienyl, or benzothienyl substituted with 1 R¹¹ group selected from fluoro, chloro, bromo, and iodo. In certain embodiments, Ar¹ is 2-pyridyl, 3-pyridyl, indazolyl, thienyl, or benzothienyl substituted with 1 R¹¹ group selected from fluoro and chloro.

In certain embodiments, Ar¹ is 2-pyridyl or 3-pyridyl, each optionally substituted with aryl optionally substituted with halo. In certain embodiments, Ar¹ is 2-pyridyl or 3-pyridyl, each substituted with fluorophenyl.

In certain embodiments, Ar¹ is indazolyl optionally substituted with (cycloalkyl)alkyl. In certain embodiments, Ar¹ is indazolyl substituted with cyclopentylmethyl.

In certain embodiments, Ar¹ is thienyl optionally substituted with cycloalkyloxyalkyl. In certain embodiments, Ar¹ is thienyl substituted with cyclopentyloxymethyl.

In certain embodiments, Ar¹ is benzothiophenyl optionally substituted with cycloalkyloxy. In certain embodiments, Ar¹ is benzothiophenyl substituted with cyclopentyloxy.

In certain embodiments, L is C(O)—C(O), R³ is H, and Ar¹ is aryl or heteroaryl, wherein the aryl and heteroaryl are each independently substituted with 1 or 2 R¹¹ groups, wherein R¹¹ is alkoxy, cycloalkyloxy, (cycloalkyl)alkyloxy, or aryl optionally substituted with halo. In certain embodiments, L is C(O)—C(O), R³ is H, and Ar¹ is aryl or heteroaryl, wherein the aryl and heteroaryl are each independently substituted with 1 R¹¹ group, wherein R¹¹ is alkoxy, cycloalkyloxy, (cycloalkyl)alkyloxy, or aryl optionally substituted with halo. In certain embodiments, L is C(O)—C(O), R³ is H, and Ar¹ is aryl or heteroaryl, wherein the aryl and heteroaryl are each independently substituted with 1 R¹¹ group, wherein R¹¹ is i-propoxy, cyclopentyloxy, fluorophenyl, or cyclohexylmethoxy. In certain embodiments, L is C(O)—C(O), R³ is H, and Ar¹ is unsubstituted aryl or unsubstituted heteroaryl.

In certain embodiments, L is C(O)—C(O), R³ is H, and Ar¹ is phenyl, naphthyl, pyridyl, or benzothienyl, each independently substituted with 1 or 2 R¹¹ groups, wherein R¹¹ is alkoxy, cycloalkyloxy, (cycloalkyl)alkyloxy, or aryl optionally substituted with halo. In certain embodiments, L is C(O)—C(O), R³ is H, and Ar¹ is phenyl, naphthyl, pyridyl, or benzothienyl, each independently substituted with 1 R¹¹ group, wherein R¹¹ is alkoxy, cycloalkyloxy, (cycloalkyl)alkyloxy, or aryl optionally substituted with halo. In certain embodiments, L is C(O)—C(O), R³ is H, and Ar¹ is phenyl, naphthyl, pyridyl, or benzothienyl, each independently substituted with 1 R¹¹ group, wherein R¹¹ is i-propoxy, cyclopentyloxy, fluorophenyl, or cyclohexylmethoxy. In certain embodiments, L is C(O)—C(O), R³ is H, and Ar¹ is unsubstituted phenyl, unsubstituted naphthyl, unsubstituted pyridyl, or unsubstituted benzothienyl.

In certain embodiments, L is SO₂, R³ is H, and Ar¹ is aryl or heteroaryl, wherein the aryl and heteroaryl are each independently substituted with 1 or 2 R¹¹ groups, wherein R¹¹ is halo, alkyl, alkoxy, hydroxy, cycloalkyl, (cycloalkyl)alkyl, heterocycloalkyl, (heterocycloalkyl)alkyl, cycloalkyloxy, heterocycloalkyloxy, (cycloalkyl)alkyloxy, cycloalkyloxyalkyl, phenyl optionally substituted with halo, aryloxy optionally substituted with halo, arylalkyloxy optionally substituted with halo, heteroaryl, or heteroaryloxy optionally substituted with halo. In certain embodiments, L is SO₂, R³ is H, and Ar¹ is aryl or heteroaryl, wherein the aryl and heteroaryl are each independently substituted with 1 R¹¹ group, wherein R¹¹ is halo, alkyl, alkoxy, hydroxy, cycloalkyl, (cycloalkyl)alkyl, heterocycloalkyl, (heterocycloalkyl)alkyl, cycloalkyloxy, heterocycloalkyloxy, (cycloalkyl)alkyloxy, cycloalkyloxyalkyl, phenyl optionally substituted with halo, aryloxy optionally substituted with halo, arylalkyloxy optionally substituted with halo, heteroaryl, or heteroaryloxy optionally substituted with halo. In certain embodiments, L is SO₂, R³ is H, and Ar¹ is phenyl, naphthyl, pyridyl, or benzothienyl, each independently substituted 1 R¹¹ group, wherein R¹¹ is halo, alkyl, alkoxy, hydroxy, cycloalkyl, (cycloalkyl)alkyl, heterocycloalkyl, (heterocycloalkyl)alkyl, cycloalkyloxy, heterocycloalkyloxy, (cycloalkyl)alkyloxy, cycloalkyloxyalkyl, phenyl optionally substituted with halo, aryloxy optionally substituted with halo, arylalkyloxy optionally substituted with halo, heteroaryl, or heteroaryloxy optionally substituted with halo. In certain embodiments, L is SO₂, R³ is H, and Ar¹ is aryl or heteroaryl, wherein the aryl and heteroaryl are each independently substituted with 1 R¹¹ group, wherein R¹¹ is alkoxy, cycloalkyloxy, heterocycloalkyloxy, (cycloalkyl)alkyloxy, cycloalkyloxyalkyl, phenyl optionally substituted with halo, aryloxy optionally substituted with halo, arylalkyloxy optionally substituted with halo, or heteroaryloxy optionally substituted with halo. In certain embodiments, L is SO₂, R³ is H, and Ar¹ is phenyl, naphthyl, pyridyl, or benzothienyl, each independently substituted 1 R¹¹ group, wherein R¹¹ is alkoxy, cycloalkyloxy, heterocycloalkyloxy, (cycloalkyl)alkyloxy, cycloalkyloxyalkyl, phenyl optionally substituted with halo, aryloxy optionally substituted with halo, arylalkyloxy optionally substituted with halo, or heteroaryloxy optionally substituted with halo. In certain embodiments, L is SO₂, R³ is H, and Ar¹ is phenyl, naphthyl, pyridyl, or benzothienyl, each independently substituted with 1 R¹¹ group, wherein R¹¹ is alkoxy, cycloalkyloxy, (cycloalkyl)alkyloxy, or phenyl optionally substituted with halo. In certain embodiments, L is SO₂, R³ is H, and Ar¹ is unsubstituted aryl or unsubstituted heteroaryl.

In certain embodiments, L is C*H(OH)C(O) wherein “C*” forms a covalent bond with Ar¹, R³ is H, and Ar¹ is phenyl or naphthyl, wherein the phenyl or napthyl is substituted with alkoxy, cycloalkyloxy, or (cycloalkyl)alkyloxy. In certain embodiments, L is C*H(OH)C(O) wherein “C*” forms a covalent bond with Ar¹, R³ is H, and Ar¹ is phenyl or naphthyl, wherein the phenyl or napthyl is substituted with i-propoxy, cyclopentyloxy, or (cyclohexyl)methoxy. In certain embodiments, L is C*H(OH)C(O) wherein “C*” forms a covalent bond with Ar¹, R³ is H, and Ar¹ is unsubstituted phenyl or unsubstituted naphthyl.

In certain embodiments, L is C*H(OH)C(O) wherein “C*” forms a covalent bond with Ar¹, R³ is H, and Ar¹ is phenyl substituted with alkoxy, cycloalkyloxy, or (cycloalkyl)alkyloxy. In certain embodiments, L is C*H(OH)C(O) wherein “C*” forms a covalent bond with Ar¹, R³ is H, and Ar¹ is phenyl substituted with i-propoxy, cyclopentyloxy, or (cyclohexyl)methoxy. In certain embodiments, L is C*H(OH)C(O) wherein “C*” forms a covalent bond with Ar¹, R³ is H, and Ar¹ is unsubstituted phenyl.

In certain embodiments, Ar² is optionally substituted aryl or optionally substituted heteroaryl. In certain embodiments, Ar² is unsubstituted aryl or unsubstituted heteroaryl. In certain embodiments, Ar² is substituted aryl or unsubstituted heteroaryl. In certain embodiments, Ar² is substituted aryl or substituted heteroaryl. In certain embodiments, Ar² is unsubstituted aryl or substituted heteroaryl.

In certain embodiments, the Ar² group is unsubstituted. In certain embodiments, the Ar² group is substituted with 1 or 2 R¹² groups. In certain embodiments, the Ar² group is substituted with 1 R¹² group. In certain embodiments, the Ar² group is substituted with 2 R¹² groups.

In certain embodiments, Ar² is aryl optionally substituted with 1 or 2 R¹² groups. In certain embodiments, Ar² is aryl optionally substituted with 1 R¹² group. In certain embodiments, Ar² is aryl optionally substituted with 2 R¹² groups. In certain embodiments, Ar² is unsubstituted aryl. In certain embodiments, Ar² is aryl substituted with 1 or 2 R¹² groups. In certain embodiments, Ar² is aryl substituted with 1 R¹² group. In certain embodiments, Ar² is aryl substituted with 2 R¹² groups. In certain embodiments, Ar² is phenyl or naphthyl, wherein each is optionally substituted with 1 or 2 R¹² groups. In certain embodiments, Ar² is phenyl or naphthyl, wherein each is optionally substituted with 1 R¹² group. In certain embodiments, Ar² is phenyl or naphthyl, wherein each is optionally substituted with 2 R¹² groups. In certain embodiments, Ar² is unsubstituted phenyl or unsubstituted naphthyl. In certain embodiments, Ar² is phenyl or naphthyl, wherein each is substituted with 1 or 2 R¹² groups. In certain embodiments, Ar² is phenyl or naphthyl, wherein each is substituted with 1 R¹² group. In certain embodiments, Ar² is phenyl or naphthyl, wherein each is substituted with 2 R¹² groups. In certain embodiments, Ar² is phenyl optionally substituted with 1 or 2 R¹² groups. In certain embodiments, Ar² is phenyl optionally substituted with 1 R¹² group. In certain embodiments, Ar² is phenyl optionally substituted with 2 R¹² groups. In certain embodiments, Ar² is unsubstituted phenyl. In certain embodiments, Ar² is phenyl substituted with 1 or 2 R¹² groups. In certain embodiments, Ar² is phenyl substituted with 1 R¹² group. In certain embodiments, Ar² is phenyl substituted with 2 R¹² groups. In certain embodiments, Ar² is naphthyl optionally substituted with 1 or 2 R¹² groups. In certain embodiments, Ar² is naphthyl optionally substituted with 1 R¹² group. In certain embodiments, Ar² is naphthyl optionally substituted with 2 R¹² groups. In certain embodiments, Ar² is unsubstituted naphthyl. In certain embodiments, Ar² is naphthyl substituted with 1 or 2 R¹² groups. In certain embodiments, Ar² is naphthyl substituted with 1 R¹² group. In certain embodiments, Ar² is naphthyl substituted with 2 R¹² groups.

In certain embodiments, Ar² is phenyl substituted with 2 R¹² groups, wherein each R¹² is independently halo, alkoxy, cycloalkyloxy, phenyl or heteroaryl, wherein the phenyl and heteroaryl are each optionally substituted with 1 or 2 halo.

In certain embodiments, Ar² is heteroaryl optionally substituted with 1 or 2 R¹² groups. In certain embodiments, Ar² is heteroaryl optionally substituted with 1 R¹² group. In certain embodiments, Ar² is heteroaryl optionally substituted with 2 R¹² groups. In certain embodiments, Ar² is unsubstituted heteroaryl. In certain embodiments, Ar² is heteroaryl substituted with 1 or 2 R¹² groups. In certain embodiments, Ar² is heteroaryl substituted with 1 R¹² group. In certain embodiments, Ar² is heteroaryl optionally substituted with 2 R¹² groups. In certain embodiments, Ar² is a 5-6 membered heteroaryl or a 9-10 membered bicyclic heteroaryl, wherein each heteroaryl is optionally substituted with 1 or 2 R¹² groups. In certain embodiments, Ar² is a 5-6 membered heteroaryl or a 9-10 membered bicyclic heteroaryl, wherein each heteroaryl is optionally substituted with 1 R¹² group. In certain embodiments, Ar² is a 5-6 membered heteroaryl or a 9-10 membered bicyclic heteroaryl, wherein each heteroaryl is optionally substituted with 2 R¹² groups. In certain embodiments, Ar² is an unsubstituted 5-6 membered heteroaryl or an unsubstituted 9-10 membered bicyclic heteroaryl. In certain embodiments, Ar² is a 5-6 membered heteroaryl or a 9-10 membered bicyclic heteroaryl, wherein each heteroaryl is substituted with 1 or 2 R¹² groups. In certain embodiments, Ar² is a 5-6 membered heteroaryl or a 9-10 membered bicyclic heteroaryl, wherein each heteroaryl is substituted with 1 R¹² group. In certain embodiments, Ar² is a 5-6 membered heteroaryl or a 9-10 membered bicyclic heteroaryl, wherein each heteroaryl is substituted with 2 R¹² groups. In certain embodiments, Ar² is a 5-6 membered heteroaryl or a 9-10 membered bicyclic heteroaryl, each containing 1 or 2 ring heteroatoms, wherein each is optionally substituted with 1 or 2 R¹² groups. In certain embodiments, Ar² is a 5-6 membered heteroaryl or a 9-10 membered bicyclic heteroaryl, each containing 1 or 2 ring heteroatoms, wherein each is optionally substituted with 1 R¹² group. In certain embodiments, Ar² is a 5-6 membered heteroaryl or a 9-10 membered bicyclic heteroaryl, each containing 1 or 2 ring heteroatoms, wherein each is optionally substituted with 2 R¹² groups. In certain embodiments, Ar² is an unsubstituted 5-6 membered heteroaryl or an unsubstituted 9-10 membered bicyclic heteroaryl, each containing 1 or 2 ring heteroatoms. In certain embodiments, Ar² is a 5-6 membered heteroaryl or a 9-10 membered bicyclic heteroaryl, each containing 1 or 2 ring heteroatoms, wherein each is substituted with 1 or 2 R¹² groups. In certain embodiments, Ar² is a 5-6 membered heteroaryl or a 9-10 membered bicyclic heteroaryl, each containing 1 or 2 ring heteroatoms, wherein each is substituted with 1 R¹² group. In certain embodiments, Ar² is a 5-6 membered heteroaryl or a 9-10 membered bicyclic heteroaryl, each containing 1 or 2 ring heteroatoms, wherein each is substituted with 2 R¹² groups. In certain embodiments, Ar² is a 5-6 membered heteroaryl or a 9-10 membered bicyclic heteroaryl, each containing 1 ring heteroatom, wherein each heteroaryl is optionally substituted with 1 or 2 R¹² groups. In certain embodiments, Ar² is a 5-6 membered heteroaryl or a 9-10 membered bicyclic heteroaryl, each containing 1 ring heteroatom, wherein each heteroaryl is optionally substituted with 1 R¹² group. In certain embodiments, Ar² is a 5-6 membered heteroaryl or a 9-10 membered bicyclic heteroaryl, each containing 1 ring heteroatom, wherein each heteroaryl is optionally substituted with 2 R¹² groups. In certain embodiments, Ar² is a 5-6 membered heteroaryl containing 1 ring heteroatom or a 9-10 membered bicyclic heteroaryl containing 1 or 2 ring heteroatoms, wherein each is optionally substituted with 1 or 2 R¹² groups. In certain embodiments, Ar² is a 5-6 membered heteroaryl containing 1 ring heteroatom or a 9-10 membered bicyclic heteroaryl containing 2 ring heteroatoms, wherein each is optionally substituted with 1 or 2 R¹² groups. In certain embodiments, Ar² is a 5-6 membered heteroaryl containing 1 or 2 ring heteroatoms, wherein the heteroaryl is optionally substituted with 1 or 2 R¹² groups. In certain embodiments, Ar² is a 5-6 membered heteroaryl containing 1 or 2 ring heteroatoms, wherein the heteroaryl is optionally substituted with 1 R¹² group. In certain embodiments, Ar² is a 5-6 membered heteroaryl containing 1 or 2 ring heteroatoms, wherein the heteroaryl is optionally substituted with 2 R¹² groups. In certain embodiments, Ar² is a 5-6 membered heteroaryl containing 1 ring heteroatom, wherein the heteroaryl is optionally substituted with 1 or 2 R¹² groups. In certain embodiments, Ar² is a 9-10 membered bicyclic heteroaryl containing 1 or 2 ring heteroatoms, wherein the heteroaryl is optionally substituted with 1 or 2 R¹² groups. In certain embodiments, Ar² is a 9-10 membered bicyclic heteroaryl containing 1 or 2 ring heteroatoms, wherein the heteroaryl is optionally substituted with 1 R¹² group. In certain embodiments, Ar² is a 9-10 membered bicyclic heteroaryl containing 1 or 2 ring heteroatoms, wherein the heteroaryl is optionally substituted with 2 R¹² groups. In certain embodiments, Ar² is a 9-10 membered bicyclic heteroaryl containing 1 ring heteroatom, wherein the heteroaryl is optionally substituted with 1 or 2 R¹² groups. In certain embodiments, Ar² is a 9-10 membered bicyclic heteroaryl containing 2 ring heteroatoms, wherein the heteroaryl is optionally substituted with 1 or 2 R¹² groups. In certain embodiments, the heteroaryl contains 1 or 2 ring nitrogen atoms or a ring sulfur atom. In certain embodiments, the heteroaryl contains a ring nitrogen atom and a ring sulfur atom. In certain embodiments, the heteroaryl contains 1 or 2 ring nitrogen atoms. In certain embodiments, the heteroaryl contains a ring sulfur atom. In certain embodiments, the heteroaryl contains 1 ring nitrogen atom. In certain embodiments, the heteroaryl contains 2 ring nitrogen atoms. In certain embodiments, Ar² is 2-pyridyl, thiophene, or dibenzofuranyl, wherein each is optionally substituted with 1 or 2 R¹² groups.

In certain embodiments, each R¹² is independently cycloalkyloxy, cycloalkyl, heterocycloalkyl, cycloalkyl substituted with haloalkyl, (cycloalkyl)alkyloxy, heterocycloalkyloxy, (heterocycloalkyl)alkyloxy, aryl, heteroaryl, aryloxy, arylalkyloxy, heteroaryloxy, or heteroarylalkyloxy; wherein each aryl and heteroaryl, whether alone or as part of another group, are each independently optionally substituted with 1 or 2 groups selected from halo, alkyl, haloalkyl, and alkoxy.

In certain embodiments, each R¹² is independently alkoxy, cycloalkyloxy, (cycloalkyl)alkyloxy, heterocycloalkyloxy, (heterocycloalkyl)alkyloxy, aryloxy, arylalkyloxy, heteroaryloxy, or heteroarylalkyloxy; wherein each aryl and heteroaryl, whether alone or as part of another group, are each independently optionally substituted with 1 or 2 groups selected from halo, alkyl, haloalkyl, and alkoxy.

In certain embodiments, each R¹² is independently halo, hydroxy, alkyl, haloalkyl, haloalkenyl, alkoxy, cycloalkyloxy, cycloalkyl substituted with haloalkyl, (heterocycloalkyl)alkyloxy, aryl, heteroaryl, aryloxy, or arylalkyloxy; wherein each aryl and heteroaryl, whether alone or as part of another group, are each independently optionally substituted with 1 or 2 groups selected from halo and haloalkyl.

In certain embodiments, each R¹² is independently halo, hydroxy, haloalkyl, haloalkenyl, alkoxy, cycloalkyloxy, cycloalkyl substituted with haloalkyl, (heterocycloalkyl)alkyloxy, aryl, heteroaryl, aryloxy, or arylalkyloxy; wherein each aryl and heteroaryl, whether alone or as part of another group, are each independently optionally substituted with 1 or 2 groups selected from halo, and haloalkyl. In certain embodiments, each R¹² is independently fluoro, chloro, bromo, iodo, hydroxy, methoxy, ethoxy, propoxy, i-propoxy, butoxy, cyclopropyloxy, cyclopentyloxy, cyclohexyloxy, trifluoromethyl, 3,3,3-trifluoropropenyl, (trifluromethyl)cyclopropyl, phenyl, chlorophenyl, trifluoromethylphenyl, 4-chlorophenoxy, (piperidinyl)ethoxy, (morpholinyl)ethoxy, 2-chlorothiophene, phenoxy, or phenylmethoxy. In certain embodiments, each R¹² is independently fluoro, chloro, bromo, iodo, hydroxy, methoxy, ethoxy, propoxy, i-propoxy, butoxy, cyclopropyloxy, cyclopentyloxy, cyclohexyloxy, trifluoromethyl, difluoromethyl, 3,3,3-trifluoropropenyl, (trifluromethyl)cyclopropyl, phenyl, fluorophenyl, chlorophenyl, trifluoromethylphenyl, difluoromethylphenyl, fluorophenoxy, chlorophenoxy, (piperidinyl)ethoxy, (morpholinyl)ethoxy, (piperidinyl)methoxy, (morpholinyl)methoxy, chlorothiophene, phenoxy, or phenylmethoxy. In certain embodiments, each R¹² is independently chloro, bromo, iodo, hydroxy, methoxy, ethoxy, i-propoxy, butoxy, cyclopropyloxy, cyclopentyloxy, cyclohexyloxy, trifluoromethyl, 3,3,3-trifluoropropenyl, (trifluromethyl)cyclopropyl, phenyl, chlorophenyl, (piperidinyl)ethoxy, (morpholinyl)ethoxy, 2-chlorothiophene, phenoxy, or phenylmethoxy.

In certain embodiments, each R¹² is independently halo, cyano, nitro, hydroxy, alkyl, haloalkyl, haloalkenyl, or alkoxy.

In certain embodiments, each R¹² is independently halo, alkoxy, cycloalkyloxy, or aryl, wherein the aryl is optionally substituted with 1 or 2 halo.

In certain embodiments, each R¹² is independently bromo, chloro, methoxy, ethoxy, propoxy, i-propoxy, butoxy, cyclopropyloxy, cyclopentyloxy, or chlorophenyl.

In certain embodiments, each R¹² is independently chloro, methoxy, butoxy, or chlorophenyl.

In certain embodiments, R¹² is halo. In certain embodiments, R¹² is bromo or chloro.

In certain embodiments, R¹² is aryl. In certain embodiments, R¹² is aryl optionally substituted with 1 or 2 groups idependently selected from halo, haloalkyl, and alkoxy. In certain embodiments, R¹² is phenyl, trifluoromethylphenyl, fluorophenyl, or chlorophenyl. In certain embodiments, R¹² is chlorophenyl.

In certain embodiments, R¹² is alkoxy. In certain embodiments, R¹² is methoxy or butoxy.

In certain embodiments, R¹² is heteroaryl. In certain embodiments, R¹² is hetereoaryl optionally substituted with 1 or 2 halo. In certain embodiments, R¹² is 2-chlorothiophene.

In certain embodiments of R¹², where each aryl and heteroaryl, whether alone or as part of another group, are each independently optionally substituted with halo, alkyl, haloalkyl, or alkoxy, such embodiments include, for example, aryl, aryloxy, arylalkyloxy, heteroaryl, heteroaryloxy, and heteroarylalkyloxy optionally substituted with halo, alkyl, haloalkyl, or alkoxy.

In certain embodiments, Ar² is independently optionally substituted with 1 or 2 R¹² groups, wherein R¹² is halo, hydroxy, alkyl, haloalkyl, haloalkenyl, alkoxy, cycloalkyloxy, cycloalkyl, heterocycloalkyl, cycloalkyl substituted with haloalkyl, (cycloalkyl)alkyloxy, heterocycloalkyloxy, (heterocycloalkyl)alkyloxy, aryl, heteroaryl, aryloxy, arylalkyloxy, heteroaryloxy, or heteroarylalkyloxy; wherein each aryl and heteroaryl, whether alone or as part of another group, are each independently optionally substituted with 1 or 2 groups selected from halo, alkyl, haloalkyl, and alkoxy.

In certain embodiments, Ar² is phenyl substituted with 1 or 2 R¹² groups, wherein R¹² is independently halo, hydroxy, haloalkyl, haloalkenyl, alkoxy, cycloalkyloxy, cycloalkyl substituted with haloalkyl, (heterocycloalkyl)alkyloxy, aryl, heteroaryl, aryloxy, or arylalkyloxy; wherein each aryl and heteroaryl, whether alone or as part of another group, are each independently optionally substituted with 1 or 2 groups selected from halo and haloalkyl.

In certain embodiments, Ar² is phenyl substituted with 1 R¹² group, wherein R¹² is halo, hydroxy, haloalkyl, haloalkenyl, alkoxy, cycloalkyloxy, cycloalkyl substituted with haloalkyl, (heterocycloalkyl)alkyloxy, aryl, heteroaryl, aryloxy, or arylalkyloxy; wherein each aryl and heteroaryl, whether alone or as part of another group, are each independently optionally substituted with 1 or 2 groups selected from halo, and haloalkyl.

In certain embodiments, Ar² is phenyl substituted with 1 or 2 R¹² groups, or Ar² is phenyl substituted with 1 R¹² group, or Ar² is phenyl substituted with 2 R¹² groups wherein each R¹² is independently fluoro, chloro, bromo, iodo, hydroxy, methoxy, ethoxy, propoxy, i-propoxy, butoxy, cyclopropyloxy, cyclopentyloxy, cyclohexyloxy, trifluoromethyl, difluoromethyl, 3,3,3-trifluoropropenyl, (trifluromethyl)cyclopropyl, phenyl, fluorophenyl, chlorophenyl, trifluoromethylphenyl, difluoromethylphenyl, fluorophenoxy, chlorophenoxy, (piperidinyl)ethoxy, (morpholinyl)ethoxy, (piperidinyl)methoxy, (morpholinyl)methoxy, chlorothiophene, phenoxy, or phenylmethoxy. In certain embodiments, Ar² is phenyl substituted with 1 or 2 R¹² groups, or Ar² is phenyl substituted with 1 R¹² group, or Ar² is phenyl substituted with 2 R¹² groups wherein each R¹² is independently chloro, bromo, iodo, hydroxy, methoxy, ethoxy, i-propoxy, butoxy, cyclopropyloxy, cyclopentyloxy, cyclohexyloxy, trifluoromethyl, 3,3,3-trifluoropropenyl, (trifluromethyl)cyclopropyl, phenyl, chlorophenyl, (piperidinyl)ethoxy, (morpholinyl)ethoxy, 2-chlorothiophene, phenoxy, or phenylmethoxy.

In certain embodiments, Ar² is phenyl substituted with 2 R¹² groups, wherein each R¹² is independently halo, alkoxy, cycloalkyloxy, or aryl, wherein aryl is independently optionally substituted with 1 or 2 halo.

In certain embodiments, Ar² is phenyl substituted with 2 R¹² groups, wherein R¹² is independently halo, alkoxy, cycloalkyloxy, phenyl, or heteroaryl, wherein the phenyl and heteroaryl are each optionally substituted with 1 or 2 halo.

In certain embodiments, L is C(O)—C(O), R³ is H, and Ar² is aryl substituted with 1 or 2 R¹² groups, wherein R¹² is halo, haloalkyl, alkoxy, cycloalkyloxy, (cycloalkyl)alkyloxy, phenyl optionally substituted with halo, or phenoxy. In certain embodiments, L is C(O)—C(O), R³ is H, and Ar² is aryl substituted with 1 or 2 R¹² groups, wherein R¹² is fluoro, chloro, bromo, trifluoromethyl, difluroromethyl, methoxy, ethoxy, propoxy, i-propoxy, butoxy, cyclopentyloxy, cyclohexyloxy, cyclopentylmethoxy cyclohexylmethoxy, fluorophenyl, chlorophenyl, bromophenyl, or benzyloxy. In certain embodiments, L is C(O)—C(O), R³ is H, and Ar² is unsubstituted aryl.

In certain embodiments, L is C(O)—C(O), R³ is H, and Ar² is phenyl or naphthyl, each independently substituted with 1 or 2 R¹² groups, wherein R¹² is halo, haloalkyl, alkoxy, cycloalkyloxy, (cycloalkyl)alkyloxy, phenyl optionally substituted with halo, or phenoxy. In certain embodiments, L is C(O)—C(O), R³ is H, and Ar² is phenyl or naphthyl, each independently substituted with 1 or 2 R¹² groups, wherein R¹² is fluoro, chloro, bromo, trifluoromethyl, difluroromethyl, methoxy, ethoxy, propoxy, i-propoxy, butoxy, cyclopentyloxy, cyclohexyloxy, cyclopentylmethoxy cyclohexylmethoxy, fluorophenyl, chlorophenyl, bromophenyl, or benzyloxy. In certain embodiments, L is C(O)—C(O), R³ is H, and Ar² is unsubstituted phenyl or unsubstituted naphthyl.

In certain embodiments, L is C(O)—C(O), R³ is H, and Ar² is phenyl substituted with 1 or 2 R¹² groups, wherein R¹² is halo, haloalkyl, alkoxy, cycloalkyloxy, (cycloalkyl)alkyloxy, phenyl optionally substituted with halo, or phenoxy. In certain embodiments, L is C(O)—C(O), R³ is H, and Ar² is phenyl substituted with 1 or 2 R¹² groups, wherein R¹² is fluoro, chloro, bromo, trifluoromethyl, difluroromethyl, methoxy, ethoxy, propoxy, i-propoxy, butoxy, cyclopentyloxy, cyclohexyloxy, cyclopentylmethoxy cyclohexylmethoxy, fluorophenyl, chlorophenyl, bromophenyl, or benzyloxy. In certain embodiments, L is C(O)—C(O), R³ is H, and Ar² is unsubstituted phenyl.

In certain embodiments, L is SO₂, R³ is H, and Ar² is aryl or heteroaryl, wherein the aryl and heteroaryl are each independently substituted with 1 or 2 R¹² groups, wherein each R¹² is independently halo, hydroxy, haloalkyl, alkoxy, cycloalkyloxy, (heterocycloalkyl)alkyloxy, aryl, heteroaryl, aryloxy, or arylalkyloxy, wherein each aryl and heteroaryl, whether alone or as part of another group, are each independently optionally substituted with 1 or 2 groups selected from halo and haloalkyl. In certain embodiments, L is SO₂, R³ is H, and Ar² is phenyl or naphthyl, or Ar² is phenyl, wherein Ar² is substituted with 1 or 2 R¹² groups, wherein each R¹² is independently halo, hydroxy, haloalkyl, alkoxy, cycloalkyloxy, (heterocycloalkyl)alkyloxy, aryl, heteroaryl, aryloxy, or arylalkyloxy, wherein each aryl and heteroaryl, whether alone or as part of another group, are each independently optionally substituted with 1 or 2 groups selected from halo and haloalkyl. In certain embodiments, L is SO₂, R³ is H, and Ar² is phenyl substituted with 1 or 2 R¹² groups, wherein R¹² is fluoro, chloro, bromo, trifluoromethyl, difluroromethyl, methoxy, ethoxy, propoxy, i-propoxy, butoxy, cyclopentyloxy, cyclohexyloxy, cyclopentylmethoxy cyclohexylmethoxy, fluorophenyl, chlorophenyl, bromophenyl, or benzyloxy. In certain embodiments, L is SO₂, R³ is H, and Ar² is unsubstituted aryl or unsubstituted heteroaryl.

In certain embodiments, L is C*H(OH)C(O) wherein “C*” forms a covalent bond with Ar¹, R³ is H, and Ar² is aryl substituted with 1 or 2 R¹² groups, wherein R¹² is halo, haloalkyl, alkoxy, cycloalkyloxy, (cycloalkyl)alkyloxy, phenyl optionally substituted with halo, or phenoxy. In certain embodiments, L is C*H(OH)C(O) wherein “C*” forms a covalent bond with Ar¹, R³ is H, and Ar² is aryl substituted with 1 or 2 R¹² groups, wherein R¹² is fluoro, chloro, bromo, trifluoromethyl, difluroromethyl, methoxy, ethoxy, propoxy, i-propoxy, butoxy, cyclopentyloxy, cyclohexyloxy, cyclopentylmethoxy cyclohexylmethoxy, fluorophenyl, chlorophenyl, bromophenyl, or benzyloxy. In certain embodiments, L is C*H(OH)C(O) wherein “C*” forms a covalent bond with Ar¹, R³ is H, and Ar² is unsubstituted aryl.

In certain embodiments, L is C*H(OH)C(O) wherein “C*” forms a covalent bond with Ar¹, R³ is H, and Ar² is phenyl or naphthyl, wherein each is substituted with 1 or 2 R¹² groups, wherein R¹² is halo, haloalkyl, alkoxy, cycloalkyloxy, (cycloalkyl)alkyloxy, phenyl optionally substituted with halo, or phenoxy. In certain embodiments, L is C*H(OH)C(O) wherein “C*” forms a covalent bond with Ar¹, R³ is H, and Ar² is phenyl or naphthyl, wherein each is substituted with 1 or 2 R¹² groups, wherein R¹² is fluoro, chloro, bromo, trifluoromethyl, difluroromethyl, methoxy, ethoxy, propoxy, i-propoxy, butoxy, cyclopentyloxy, cyclohexyloxy, cyclopentylmethoxy cyclohexylmethoxy, fluorophenyl, chlorophenyl, bromophenyl, or benzyloxy. In certain embodiments, L is C*H(OH)C(O) wherein “C*” forms a covalent bond with Ar¹, R³ is H, and Ar² is unsubstituted phenyl or unsubstituted naphthyl.

In certain embodiments, L is S(O)₂; R¹ and R², each is independently alkyl or aminoalkyl; or R¹ and R² together with the nitrogen to which they are attached form a 4-8 membered heterocycloalkyl ring optionally substituted with 1, 2, or 3 R¹⁰ groups; or a bicyclic heteroaryl ring with 8 ring atoms, where 1-3 ring atoms are nitrogen; R³ is H, alkyl, or haloalkyl; Ar¹ is aryl or heteroaryl, each optionally substituted with 1 or 2 R¹¹ groups; Ar² is aryl or heteroaryl, each optionally substituted with 1 or 2 R¹² groups; each R¹⁰ is independently halo, cyano, amino, dialkylamino, aminoalkyl, aminocarbonyl, or C(═NOH)(NH₂); each R¹¹ is independently halo, alkyl, alkoxy, (cycloalkyl)alkyl, cycloalkyloxy, (cycloalkyl)alkyloxy, cycloalkyloxyalkyl, aryl, aryloxy, arylalkyloxy, or heteroaryloxy; wherein each aryl and heteroaryl, whether alone or as part of another group, are each independently optionally substituted with halo; and each R¹² is independently halo, hydroxy, haloalkyl, haloalkenyl, alkoxy, cycloalkyloxy, cycloalkyl substituted with haloalkyl, (heterocycloalkyl)alkyloxy, aryl, heteroaryl, aryloxy, or arylalkyloxy, wherein each aryl and heteroaryl, whether alone or as part of another group, are each independently optionally substituted with 1 or 2 groups selected from halo, haloalkyl, and alkoxy.

In certain embodiments, L is C(O)—C(O); R¹ and R² together with the nitrogen to which they are attached form a 6-8 membered heterocycloalkyl ring optionally substituted with 1 R¹⁰ group; R³ is H; Ar¹ is aryl or heteroaryl, each optionally substituted with 1 R¹¹ group; Ar² is aryl optionally substituted with 1 R¹² group; R¹⁰ is amino group; R¹¹ is alkoxy, cycloalkyloxy, (cycloalkyl)alkyloxy, or aryl, wherein the aryl is optionally subsituted with a halo group; and R¹² is halo or aryl, wherein the aryl is optionally substituted with a halo group.

In certain embodiments, L is C*H(OH)C(O), wherein the “C*” in the C*H(OH)C(O) forms a covalent bond with Ar¹; R¹ and R² together with the nitrogen to which they are attached form a 6-8 membered heterocycloalkyl ring optionally substituted with 1 R¹⁰ group; R³ is H; R¹⁰ is amino group; Ar¹ is aryl optionally substituted with 1 R¹¹ group; Ar² is aryl optionally substituted with 1 R¹² group; R¹¹ is alkoxy, cycloalkyloxy, or (cycloalkyl)alkyloxy; and R¹² is halo or aryl, wherein the aryl is optionally substituted with a halo group.

In certain embodiments, where L is C₂alkylene-S(O)₂, wherein the alkylene in the C₂alkylene-S(O)₂ forms a covalent bond with Ar¹; R¹ and R² together with the nitrogen to which they are attached form a 8 membered heterocycloalkyl ring optionally substituted with amino group; R³ is H; Ar¹ is aryl optionally substituted with alkoxy; and Ar² is aryl optionally substituted with halo group.

In certain embodiments, Ar¹ is substituted with 1 or 2 R¹¹ group at the ortho position of the Ar¹. In certain embodiments, Ar¹ is substituted with 1 or 2 R¹¹ group at the meta position of the Ar¹. In certain embodiments, Ar¹ is substituted with 1 R¹¹ group at the para position of the Ar¹. In certain embodiments, Ar¹ is substituted with 1 or 2 R¹¹ group at the ortho and the meta positions of the Ar¹. In certain embodiments, Ar¹ is substituted with 1 or 2 R¹¹ group at the ortho and the para positions of the Ar¹. In certain embodiments, Ar¹ is substituted with 1 or 2 R¹¹ group at the meta and the para positions of the Ar¹.

In certain embodiments, Ar² is substituted with 1 or 2 R¹² group at the ortho position of the Ar². In certain embodiments, Ar² is substituted with 1 or 2 R¹² group at the meta position of the Ar². In certain embodiments, Ar² is substituted with 1 R¹² group at the para position of the Ar². In certain embodiments, Ar² is substituted with 1 or 2 R¹² group at the ortho and the meta positions of the Ar². In certain embodiments, Ar² is substituted with 1 or 2 R¹² group at the ortho and the para positions of the Ar². In certain embodiments, Ar² is substituted with 1 or 2 R¹² group at the meta and the para positions of the Ar¹.

In certain embodiments, the compound is a compound of Formula (I), (Ia), (Ib), I(c), I(d), I(e), I(f), II(a), II(b), II(c), II(d), II(e), II(f), II(g), II(h), III(a), III(b), IV(a), IV(b), IV(c), IV(d), V(a), V(b), V(c), or V(d), or a single stereoisomer or mixture of stereoisomers thereof, as defined herein.

In certain embodiments, the compound is selected from the group consisting of Compounds 1-200, or a single stereoisomer or mixture of stereoisomers thereof. In certain embodiments, the compound is selected from the group consisting of the compounds 5-1, 28-1, 29-1, 32-1, 33-1, 34-1, 35-1, 36-1, 37-1, 42-1, 43-1, 44-1, 48-1, 50-1, 52-1, 54-1, 55-1, 56-1, 57-1, 59-1, 66-1, 61-1, 61-2, 71-1, 73-1, 80-1, 82-1, 83-1, 87-1, 86-1, 89-1, 90-1, 91-1, 92-1, 95-1, 100-1, 101-1, 102-1, 103-1, 105-1, 107-1, 108-1, 111-1, 112-1, 113-1, 118-1, 120-1, 121-1, 123-1, 124-1, 125-1, 127-1, 126-1, 128-1, 135-1, 138-1, 139-1, 142-1, 144-1, 145-1, 146-1, 147-1, 149-1, 150-1, 155-1, 169-1, 161-3, 176-1, 182-1, 183-1, 191-1, 192-3, 193-1, and 197-1. In certain embodiments, the compound is selected from the group consisting of the compounds 2, 3, 11, 12, 15, 17, 18, 20, 22, 23, 25, 26, 29, 30, 35, 39, 40, 50, 58, 59, 60, 63, 64, 70, 72, 79, 81, 85, 84, 96, 103, 104, 105, 109, 113, 117, 120, 125, 127, 128, 129, 134, 141, 142, 145, 146, 151, 152, 153, 155, 156, 163, 171, 179, 181, 183, 185, 186, 189, 190, 194, 193, 195, 196, and 198, or a single stereoisomer or mixture of stereoisomers thereof. In certain embodiments, the compound is selected from the group consisting of the compounds 1, 4, 5, 6, 7, 8, 9, 10D, 13, 16, 19, 21, 24, 5, 27, 28, 31, 32, 33, 34, 36, 37, 38, 41, 42, 43, 44, 45, 46, 47, 48, 49, 51, 52, 53, 54, 55, 56, 57, 62, 66, 65, 67, 68, 61, 69, 71, 73, 76, 80, 82, 83, 87, 86, 89, 90, 91, 97, 98, 100, 101, 102, 106, 107, 108, 110, 111, 112, 114, 115, 116, 118, 119, 121, 122, 123, 124, 126, 130, 131, 132, 133, 135, 136-cis, 136-trans, 137, 138, 139, 140, 143, 144, 147, 148, 200, 149, 150, 154, 157, 158, 159, 160, 161, 162, 166, 168, 169, 170, 172, 174, 173, 176, 177, 178, 180, 182-1, 182-2, 184, 187, 188, 191, 192, 197-1, and 197-2, or a single stereoisomer or mixture of stereoisomers thereof. In certain embodiments, the compound is selected from the group consisting of the compounds 199, 14, 75, 74, 77, 78, 88, 92, 93, 94, 95, 99, 164, 165, 167, and 175, or a single stereoisomer or mixture of stereoisomers thereof. In certain embodiments, the compound is selected from the group consisting of the compounds 1, 2, 3, 4, 5, 6, 7, 8, 10, 11, 12, 13, 199, 15, 16, 17, 18, 20, 21, 22, 23, 24, 25, 26, 27, 28, 29, 30, 31, 32, 33, 34, 35, 36, 37, 38, 39, 40, 42, 43, 44, 45, 46, 47, 48, 49, 50, 51, 52, 53, 54, 55, 56, 57, 58, 59, 60, 62, 63, 64, 66, 65, 67, 68, 61, 69, 70, 75, 71, 72, 73, 74, 76, 77, 78, 79, 80, 81, 82, 83, 85, 84, 87, 86, 88, 89, 90, 91, 92 93, 94, 95, 96, 97, 98, 99, 100, 101, 102, 103, 104, 105, 106, 107, 109, 108, 110, 111, 112, 113, 114, 115, 116, 117, 118, 119, 120, 121, 122, 123, 124, 125, 127, 126, 128, 129, 130, 131, 132, 135, 137, 138, 139, 140, 142, 143, 144, 145, 146, 147, 148, 200, 149, 150, 151, 153, 154, 155, 156, 157, 161, 162, 163, 164, 165, 166, 167, 168, 169, 170, 171, 172, 174, 173, 176, 177, 178, 179, 180, 181, 182-1, 182-2, 183, 184, 185, 187, 186, 188, 189, 190, 191, 192, 194, 193, 195, 196, 197-1, 197-2, and 198, or a single stereoisomer or mixture of stereoisomers thereof. In certain embodiments, the compound is selected from the group consisting of the compounds 9, 14, 133, 134, 160, and 175, or a single stereoisomer or mixture of stereoisomers thereof. In certain embodiments, the compound is selected from the group consisting of the compounds 19, 41, 136-cis, 136-trans, 141, 152, 158, and 159, or a single stereoisomer or mixture of stereoisomers thereof.

In certain embodiments, the compound is selected from Table 1.

### Pharmaceutical Compositions

In certain embodiments, optionally in combination with any or all of the above various embodiments, provided herein is a pharmaceutical composition comprising of a compound disclosed herein, for example, a compound of Formula (I), (Ia), (Ib), I(c), I(d), I(e), I(f), II(a), II(b), II(c), II(d), II(e), II(f), II(g), II(h), III(a), III(b), IV(a), IV(b), IV(c), IV(d), V(a), V(b), V(c), or V(d), or a compound selected from the group consisting of Compounds 1-200, or a compound of Table 1, or stereoisomers thereof, or a pharmaceutically acceptable salt thereof, and one or more pharmaceutically acceptable excipients.

In certain embodiments, the pharmaceutical composition comprises a compound of Formula (I), (Ia), (Ib), I(c), I(d), I(e), I(f), II(a), II(b), II(c), II(d), II(e), II(f), II(g), II(h), III(a), III(b), IV(a), IV(b), IV(c), IV(d), V(a), V(b), V(c), or V(d), or stereoisomers thereof, or a pharmaceutically acceptable salt thereof, and one or more pharmaceutically acceptable excipients. In certain embodiments, the pharmaceutical composition comprises a compound selected from the group consisting of Compounds 1-200, or stereoisomers thereof, or a pharmaceutically acceptable salt thereof, and one or more pharmaceutically acceptable excipients. In certain embodiments, the pharmaceutical composition comprises a compound of Table 1, or stereoisomers thereof, or a pharmaceutically acceptable salt thereof, and one or more pharmaceutically acceptable excipients.

Excipients include, for example, encapsulating materials or additives such as absorption accelerators, antioxidants, binders, buffers, coating agents, coloring agents, diluents, disintegrating agents, emulsifiers, extenders, fillers, flavoring agents, humectants, lubricants, perfumes, preservatives, propellants, releasing agents, sterilizing agents, sweeteners, solubilizers, wetting agents and mixtures thereof.

Suitable excipients are well known to those skilled in the art. Whether a particular excipient is suitable for incorporation into a pharmaceutical composition or dosage form depends on a variety of factors well known in the art, including, but not limited to, the method of administration. The suitability of a particular excipient may also depend on the specific active ingredients in the dosage form.

### Formulation and Administration

All the compounds and pharmaceutical compositions provided herein can be used in all the methods provided herein. For example, the compounds and pharmaceutical compositions provided herein can be used in all the methods for treatment of all diseases, disorders or conditions provided herein. Thus, the compounds and pharmaceutical compositions provided herein are for use as a medicament. The compounds and pharmaceutical compositions provided herein are for use in a method for the treatment of a disease or disorder that is mediated by the enzyme CGT. The compounds and pharmaceutical compositions provided herein are for use in a method for the treatment of a disease or disorder in which inhibition of the enzyme CGT ameliorates or treats the disease or disorder. For example, a compound provided herein is a compound of Formula (I), (Ia), (Ib), I(c), I(d), I(e), I(f), II(a), II(b), II(c), II(d), II(e), II(f), II(g), II(h), III(a), III(b), IV(a), IV(b), IV(c), IV(d), V(a), V(b), V(c), or V(d), or a compound selected from the group consisting of Compounds 1-200, or a compound of Table 1, or stereoisomers thereof, and additionally optionally a pharmaceutically acceptable salt thereof. In certain embodiments, the compounds described herein are used in the preparation or manufacture of medicaments for the treatment of a disease or disorder that is mediated by the enzyme CGT or in which inhibition of the enzyme CGT ameliorates or treats the disease or disorder. In certain embodiments, provided is a method for treating any of the diseases or disorders described herein comprising administering to a subject having the disease or disorder a compound according to any of the various embodiments described herein or a pharmaceutical composition according to any of the various embodiments described herein. In certain embodiments, provided is a method for treating any of the diseases or disorders described herein comprising administering to a subject in need of treatment thereof a compound according to any of the various embodiments described herein or a pharmaceutical composition according to any of the various embodiments described herein.

In certain embodiments, the compounds and pharmaceutical compositions provided herein are for use in a method for the treatment of a disease or disorder that is mediated by the enzyme CGT, or in which inhibition of the enzyme CGT ameliorates or treats the disease or disorder.

In certain embodiments, provided herein is a method of treating a disease or disorder ameliorated by the inhibition of CGT comprising administering to a subject having the disease or disorder a therapeutically effective amount of a compound provided herein, for example, a compound of Formula (I), (Ia), (Ib), I(c), I(d), I(e), I(f), II(a), II(b), II(c), II(d), II(e), II(f), II(g), II(h), III(a), III(b), IV(a), IV(b), IV(c), IV(d), V(a), V(b), V(c), or V(d), or a compound selected from the group consisting of Compounds 1-200, or a compound of Table 1, or stereoisomers thereof, and additionally optionally a pharmaceutically acceptable salt thereof. In certain embodiments, the method comprises administering to a subject having the disease or disorder a therapeutically effective amount of a compound of Formula (I), (Ia), (Ib), I(c), I(d), I(e), I(f), II(a), II(b), II(c), II(d), II(e), II(f), II(g), II(h), III(a), III(b), IV(a), IV(b), IV(c), IV(d), V(a), V(b), V(c), or V(d), or stereoisomers thereof, and additionally optionally a pharmaceutically acceptable salt thereof. In certain embodiments, the method comprises administering to a subject having the disease or disorder a therapeutically effective amount of a compound selected from the group consisting of Compounds 1-200, or stereoisomers thereof, and additionally optionally a pharmaceutically acceptable salt thereof. In certain embodiments, the method comprises administering to a subject having the disease or disorder a therapeutically effective amount of a compound of Table 1, or stereoisomers thereof, and additionally optionally a pharmaceutically acceptable salt thereof.

In certain embodiments, provided herein is a method of treating a disease or disorder ameliorated by the inhibition of CGT comprising administering to a subject having the disease or disorder a pharmaceutical composition comprising a therapeutically effective amount of a compound provided herein, for example, a compound of Formula (I), (Ia), (Ib), I(c), I(d), I(e), I(f), II(a), II(b), II(c), II(d), II(e), II(f), II(g), II(h), III(a), III(b), IV(a), IV(b), IV(c), IV(d), V(a), V(b), V(c), or V(d), or a compound selected from the group consisting of Compounds 1-200, or a compound of Table 1, or stereoisomers thereof, and additionally optionally a pharmaceutically acceptable salt thereof, and a pharmaceutically acceptable excipient. In certain embodiments, the method comprises administering to a subject having the disease or disorder a pharmaceutical composition comprising a therapeutically effective amount of a compound of Formula (I), (Ia), (Ib), I(c), I(d), I(e), I(f), II(a), II(b), II(c), II(d), II(e), II(f), II(g), II(h), III(a), III(b), IV(a), IV(b), IV(c), IV(d), V(a), V(b), V(c), or V(d), or stereoisomers thereof, and additionally optionally a pharmaceutically acceptable salt thereof, and a pharmaceutically acceptable excipient. In certain embodiments, the method comprises administering to a subject having the disease or disorder a pharmaceutical composition comprising a therapeutically effective amount of a compound selected from the group consisting of Compounds 1-200, or stereoisomers thereof, and additionally optionally a pharmaceutically acceptable salt thereof, and a pharmaceutically acceptable excipient. In certain embodiments, the method comprises administering to a subject having the disease or disorder a pharmaceutical composition comprising a therapeutically effective amount of a compound of Table 1, or stereoisomers thereof, and additionally optionally a pharmaceutically acceptable salt thereof, and a pharmaceutically acceptable excipient.

In certain embodiments, the disease or disorder is a lysosomal storage disease. In certain embodiments, the lysosomal storage disease or disorder is a defect in sphigolipid metabolism, Krabbe disease and Metachromatic Leukodystrophy (MLD). In certain embodiments, the lysosomal storage disease or disorder is Krabbe disease or Metachromatic Leukodystrophy (MLD). In certain embodiments, the disease or disorder is Krabbe disease. In certain embodiments, the disease or disorder is MLD. In certain embodiments, the disease or disorder is Parkinson's disease.

The compounds or compositions disclosed herein can be administered in therapeutically effective amounts via any of the usual and acceptable modes known in the art, either singly or in combination with another therapeutic agent. The compounds are typically administered as pharmaceutical compositions by any route which makes the compound bioavailable. In certain embodiments, the composition is a solid formulation adapted for oral administration. In certain embodiments, the composition is a tablet, powder, or capsule; or the composition is a tablet. In certain embodiments, the composition is a liquid formulation adapted for oral administration. In certain embodiments, the composition is a liquid formulation adapted for parenteral administration. In certain embodiments, the composition is a solution, suspension, or emulsion; or the composition is a solution. In certain embodiments, solid form compositions can be converted, shortly before use, to liquid form compositions for either oral or parenteral administration. These particular solid form compositions are provided in unit dose form and as such are used to provide a single liquid dosage unit. These dosage forms can be prepared according to conventional methods and techniques known to those skilled in the art (see, Remington: The Science and Practice of Pharmacy, supra; Modified-Release Drug Delivery Technology, 2nd ed.; Rathbone et al., Eds.; Marcel Dekker, Inc.: New York, N.Y., 2008).

The dosages may be varied depending on the requirement of the patient, the severity of the disease or disorder being treating and the particular compound and/or composition being employed. Determination of the proper dosage can be determined by one skilled in the medical arts. The total daily dosage may be divided and administered in portions throughout the day or by means providing continuous delivery. In certain embodiments, the compounds are administered to a subject at a daily dosage of between 0.01 to about 50 mg/kg of body weight. In other embodiments, the dose is from 1 to 1000 mg/day. In certain embodiments, the daily dose is from 1 to 750 mg/day; or from 10 to 500 mg/day.

In certain embodiments, the pharmaceutical composition is in unit dosage form. The composition can be subdivided into unit doses containing appropriate quantities of the active component(s). The unit dosage form can be a tablet, capsule, or powder in a vial or ampule, or it may be the appropriate number of any of these in a packaged form. The unit dosage form can be a packaged form, the package containing discrete quantities of composition such as packeted tablets, capsules, or powders in vials or ampules. The quantity of active compound(s) in a unit dose of the composition may be varied or adjusted from about 1 mg to about 100 mg, or from about 1 mg to about 50 mg, or from about 1 mg to about 25 mg.

The compounds or pharmaceutical compositions disclosed herein can be administered at once, or multiple times at intervals of time. It is understood that the precise dosage and duration of treatment may vary with the age, weight, and condition of the patient being treated, and may be determined empirically using known testing protocols or by extrapolation from in vivo or in vitro test or diagnostic data. It is further understood that for any particular individual, specific dosage regimens should be adjusted over time according to the individual need and the professional judgment of the person administering or supervising the administration of the formulations.

### Preparation of Compounds

The following are illustrative schemes and examples of how the compounds of Formula I can be prepared and tested. Although the examples can represent only some embodiments, it should be understood that the following examples are illustrative and not limiting. All substituents, unless otherwise specified, are as previously defined. The reagents and starting materials are readily available to one of ordinary skill in the art. The specific synthetic steps for each of the routes described may be combined in different ways, or in conjunction with steps from different schemes, to prepare the compounds described herein.

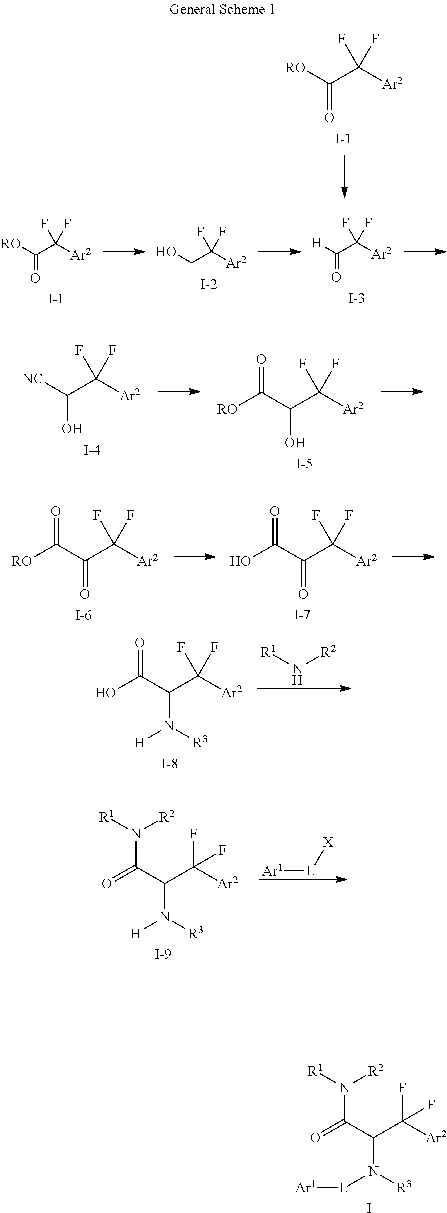

A compound of Formula I can be prepared according to General Scheme 1, where R is hydrogen or an alkyl or cycloalkyl group. In certain embodiments, R is methyl, ethyl, n-propyl, isopropyl, butyl, or cyclopropyl.

Compound I-2 can be prepared from the corresponding ester using standard reduction conditions. More specifically, reducing reagents include LiAlH₄ or NaBH₄ in a solvent such as MeOH, EtOH, or THF, optionally in the presence of CaCl₂, and at ambient temperature or up to 40° C.

Compound I-3 can be prepared from its corresponding alcohol using standard oxidation conditions. More specifically, Compound I-2 can be oxidized with an oxidizing reagent, such as Dess-Martin Periodinane, in a solvent such as CH₂Cl₂ or CH₃CN, in the presence of a base such as NaHCO₃, or pyridine, to yield Compound I-3. Compound I-3 can also be prepared from Compound I-1 using standard reduction conditions. More specifically, Compound I-1 can be reduced with a reducing reagent, such as NaBH₄ or DIBAL in a solvent such as MeOH, EtOH, or THF, and at −78° C. or up to ambient temperature.

Compound I-3 can also be converted to Compound I-4 using standard cyanohydrin reaction conditions. More specifically, Compound I-3 can be treated with NaCN, KCN, or TMSCN in a solvent such as MeOH, EtOH, or water, in the presence of an acid such as AcOH, H₂SO₄, KH₂PO₄, or Na₂S₂O₅, to yield Compound I-4.

Compound I-4 can be treated with anhydrous HCl gas in a solvent such as EtOH or MeOH, and at −20° C. or up to ambient temperature to yield Compound I-5.

Compound I-6 can be prepared using standard oxidation conditions. More specifically, Compound I-5 can be oxidized with an oxidizing reagent, such as Dess-Martin Periodinane, in a solvent such as CH₂Cl₂ or CH₃CN, in the presence of a base such as NaHCO₃, or pyridine, to yield Compound I-6.

Compound I-6 can be treated with a base such as NaHCO₃ in a solvent such as iPrOH, EtOH or MeOH, and at ambient temperature or up to 80° C. to yield Compound I-7.

Compound I-8 can be prepared using standard reductive amination conditions. More specifically, Compound I-7 can be treated with an amine of formula R³NH₂ with a reducing reagent, such as NaBH₄, NaBH₃CN, or NaBH(OAc)₃, in a solvent such as water, MeOH, or EtOH, at ambient temperature or up to 80° C. to afford Compound I-8.

Compound I-9 can be prepared using standard amide formation conditions. More specifically, Compound I-8 can be reacted with an amine of formula R¹—NH—R² and an amide coupling reagent, such as EDCI or HATU, in a solvent such as DCM or THF, in the presence of a base such as DIPEA or TEA, optionally in the presence of HOBt, at ambient temperature or up to 80° C. to yield Compound I-9.

The compound of Formula I (where L is S(O)_{q}, C₁₋₃alkylene-S(O)_{q}, C(O)—C(O), or C*H(OH)C(O), wherein the alkylene in the C₁₋₃alkylene-S(O)_{q} forms a covalent bond with Ar¹, and wherein “C*” forms a covalent bond with Ar¹, and q is 0, 1, or 2) can be prepared using standard sulfonamide or amide formation conditions. More specifically, Compound I-9 can be treated with an Ar¹-L-X, wherein X is halogen (such as Cl or Br), —OH, or a carboxylic acid, in the presence of a base, such as pyridine, DIPEA, or TEA, optionally in a solvent, such as DCM or THF, at ambient temperature or up to 80° C., to afford the compound of Formula I (where L is S(O)_{q}, and q is 0, 1, or 2), or in the presence of a amide coupling reagent, such as HATU, and a base, such as DIPEA or TEA, in a solvent, such as DCM or THF, at ambient temperature or up to 80° C. to afford the compound of Formula I.

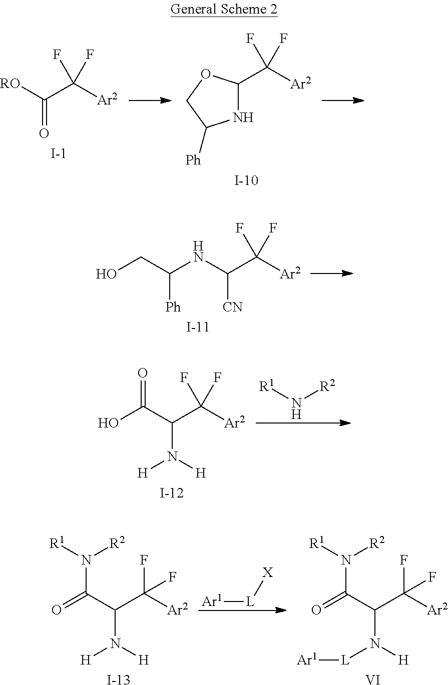

A compound of Formula VI can also be prepared according to General Scheme 2, where R is hydrogen or an alkyl or cycloalkyl group. In certain embodiments, R is methyl, ethyl, n-propyl, isopropyl, butyl, or cyclopropyl.

Compound I-10 can be prepared from the corresponding ester by a reduction reaction followed by acetal formation. More specifically, Compound I-1 can be reduced to form the corresponding aldehyde or acetal or hemiacetal with a reducing reagent, such as NaBH₄ in a solvent such as MeOH or EtOH, at −80° C. or up to ambient temperature, preferable at −50˜−60° C. Treatment of the aldehyde or acetal or hemiacetal with a substituted glycinol, such as phenylglycinol, in a solvent, such as toluene, in the presence of an acid, such as pyridinium p-toluenesulfonate or p-toluenesulfonic acid, at reflux temperature will yield Compound I-10.

Compound I-11 can be prepared using Strecker-type reaction conditions. More specifically, Compound I-10 can be treated with a cyanide, such as trimethylsilanecarbo nitrile, in a solvent such as CH₂Cl₂, in the presence of a Lewis acid or an acid, such as BF₃-Et₂O, at −80° C. or up to ambient temperature to afford Compound I-11.

Compound I-11 can be converted to Compound I-12 using oxidative hydrolysis or hydrolysis conditions. More specifically, Compound I-11 can be treated with an oxidizing reagents, such as lead (IV) acetate, in a solvent, such as CH₂Cl₂ or CHCl₃, at −10° C. or up to ambient temperature, followed by hydrolysis with an acid, such as concentrated HCl, at 60° C. or up to reflux, to yield Compound I-12.

Compound I-13 and the compound of Formula VI can also be prepared according to the procedures for Compound I-9 and the compound of Formula I in General Scheme 1.

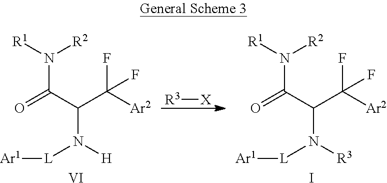

A compound of Formula I, wherein R³ is alkyl or haloalkyl, L is S(O)_{q}, and q is 0, 1, or 2, can also be prepared according to General Scheme 3.

The compound of Formula I can be prepared using standard alkylation conditions. More specifically, the compound of Formula VI can be treated with an alkylating agent, such as R³-LG (LG is an appropriate leaving group), in a solvent, such as DMSO, DMF, or THF, in the presence of a base, such as K₂CO₃ or Na₂CO₃, at −20° C. or up to ambient temperature, to yield the compound of Formula I.

### SYNTHETIC EXAMPLES

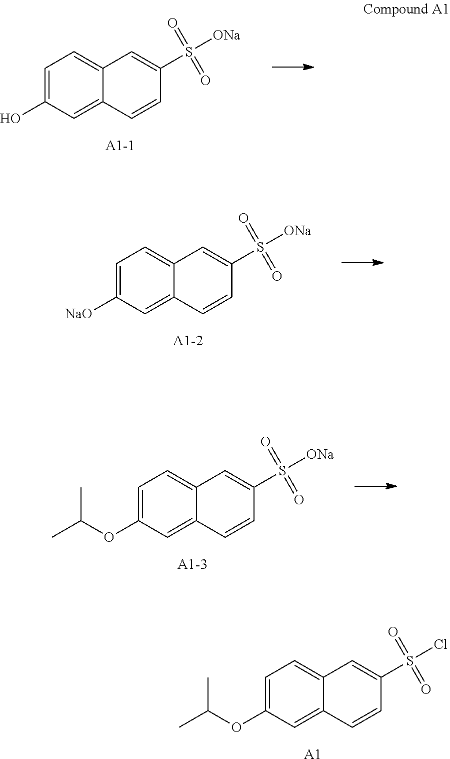

To a solution of sodium methanolate (1.6 g, 30 mmol) in ethanol (120 mL) was added sodium 6-hydroxynaphthalene-2-sulfonate A1-1 (5 g, 20 mmol) and the reaction mixture was stirred at room temperature for 2 hours. The reaction mixture was filtered and washed with methanol to give Compound A1-2.

To a solution of Compound A1-2 (1.1 g, 4.14 mmol) in DMSO (50 mL) was added 2-bromopropane (651 mg, 5.3 mmol). The mixture was stirred at room temperature (specify room temperature in specification) for 18 hours and poured into ice-cold water with continuous stirring. The precipitated solid was filtered and washed with ice-cold water to provide Compound A1-3. LC-MS (ESI) m/z: 267 [M+2H—Na]⁺; ¹H-NMR (CDCl₃, 400 MHz): δ (ppm) 1.33 (d, J=6.0 Hz, 6H), 4.77 (m, 1H), 7.13 (d, J=8.8 Hz, 1H), 7.31 (s, 1H), 7.64 (d, J=8.8 Hz, 1H), 7.73 (d, J=8.8 Hz, 1H), 7.87 (d, J=8.8 Hz, 1H), 8.05 (s, 1H).

To an ice-cooled suspension of Compound A1-3 (2.45 g, 9.2 mmol) in DMF (10 mL) was added thionyl chloride (1.3 mL, 18.4 mmol). The mixture was stirred at 18° C. for 2 hours. The reaction mixture was poured into crushed ice with continuous stirring. The resulting mixture was filtered to give Compound A1. ¹H-NMR (CDCl₃, 400 MHz): δ (ppm) 1.43 (d, J=6.0 Hz, 6H), 4.77 (m, 1H), 7.19 (s, 1H), 7.26 (d, J=8.8 Hz, 1H), 7.84-7.93 (m, 3H), 8.47 (s, 1H).

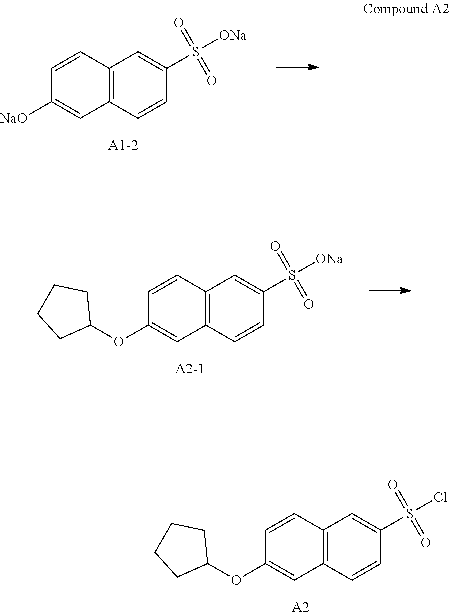

Compound A2-1 and Compound A2 were synthesized by employing the procedures described for Compounds A1-3 and A1 using bromocyclopentane and Compound A2-1 in lieu of 2-bromopropane and Compound A1-3.

Compound A2-1: LC-MS (ESI) m/z: 291 [M−Na]⁺; ¹H-NMR (DMSO-d₆, 400 MHz): δ (ppm) 1.59-1.63 (m, 2H), 1.69-1.78 (m, 4H), 1.95-2.03 (m, 2H), 4.93-4.97 (m, 1H), 7.10-7.13 (m, 1H), 7.27 (s, 1H), 7.64 (d, J=8.4 Hz, 1H), 7.73 (d, J=8.4 Hz, 1H), 7.85 (d, J=8.8 Hz, 1H), 8.05 (s, 1H).

Compound A2: LC-MS (ESI) m/z: 311 [M+H]⁺; ¹H-NMR (CDCl₃, 400 MHz): δ (ppm) 1.50-1.70 (m, 6H), 1.88-2.00 (m, 2H), 4.89-4.93 (m, 1H), 6.92-7.03 (m, 1H), 7.22 (s, 1H), 7.60 (d, J=8.4 Hz, 1H), 7.69 (d, J=8.4 Hz, 1H), 7.75 (d, J=8.8 Hz, 1H), 8.00 (s, 1H).

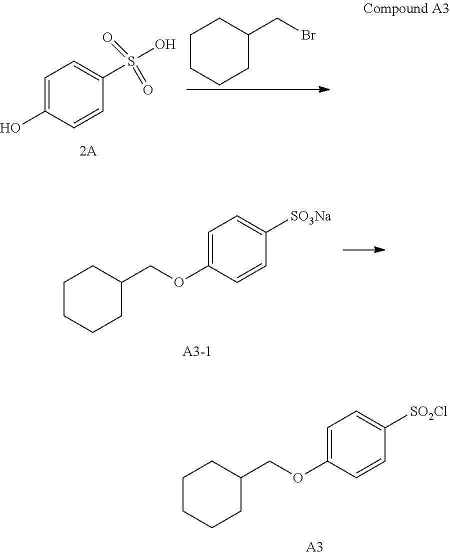

Compound A3-1 and Compound A3 were synthesized by employing the procedures described for Compound 2B and Compound A1 using (bromomethyl)cyclohexane and Compound A3-1 in lieu of 3-(bromomethyl)pentane and Compound A1-3.

Compound A3-1: LC-MS (ESI) m/z: 269 [M−Na]⁺; ¹H-NMR (DMSO-d₆, 400 MHz): δ (ppm) 0.98-1.30 (m, 5H), 1.63-1.81 (m, 6H), 3.76 (d, J=6.4 Hz, 2H), 6.83 (d, J=8.4 Hz, 2H), 7.49 (d, J=8.4 Hz, 2H).

Compound A3: ¹H-NMR (CDCl₃, 400 MHz): δ (ppm) 1.05-1.33 (m, 5H), 1.72-1.88 (m, 6H), 3.85 (d, J=6.0 Hz, 2H), 7.01 (m, 2H), 7.95 (m, 2H).

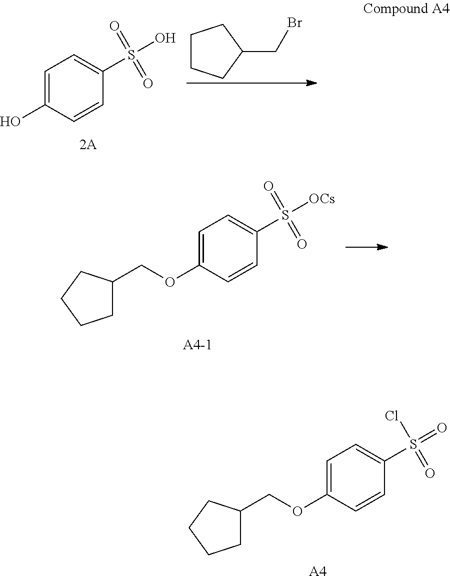

A mixture of Compound 2A (1.7 g, 10 mmol), (bromomethyl)cyclopentane (4.9 g, 30 mmol), and Cs₂CO₃ (10 g, 30 mmol) in NMP (50 mL) was heated at 145° C. overnight. The mixture was cooled down to room temperature and directly purified with reverse phase chromatography using eluent (methanol in water, 35% v/v) to give Compound A4-1. LC-MS (ESI) m/z: 255 [M−Cs]⁺.

Compound A4 was synthesized by employing the procedure described for and Compound A1 using Compound A4-1 in lieu of Compound A1-3. ¹H-NMR (CDCl₃, 400 MHz): δ (ppm) 1.27-1.32 (m, 2H), 1.54-1.60 (m, 4H), 1.76-1.83 (m, 2H), 2.29-2.38 (m, 1H), 3.86 (d, J=6.4 Hz, 2H), 6.96 (d, J=9.2 Hz, 2H), 7.89 (d, J=9.2 Hz, 2H).

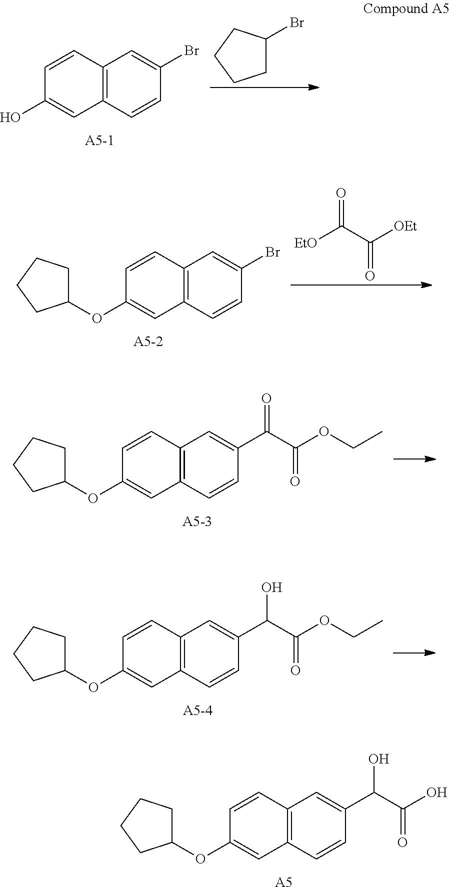

To a solution of NaH (600 mg, 15 mmol) in DMF (20 mL) under nitrogen atmosphere at 0° C. was added Compound A5-1 (2.22 g, 10 mmol) and stirred at 0° C. for 10 minutes. The reaction mixture was warmed to 25° C. and bromocyclopentane (5.3 mL, 50 mmol) was added. The reaction mixture was then stirred at 25° C. for 15 hours, quenched with water (20 mL), and extracted with ethyl acetate (40 mL×2). The combined organic extracts were washed with brine (50 mL), dried over anhydrous sodium sulfate, filtered, and concentrated to provide a residue. The residue was purified by silica gel column chromatography to yield Compound A5-2. ¹H-NMR (CDCl₃, 400 MHz): δ (ppm) 1.61-1.70 (m, 2H), 1.77-2.02 (m, 6H), 4.85-4.89 (m, 1H), 7.06 (s, 1H), 7.11 (q, J=8.8 Hz, J₂=2.4 Hz, 1H), 7.47 (q, J=8.4 Hz, 1H), 7.57 (q, J=8.8 Hz, 1H), 7.62 (q, J=8.4 Hz, 1H), 7.89 (m, 1H).

Compounds A5-3, A5-4, and A5 were synthesized by employing the procedures described for Compounds 9B, 19A, and 9C using Compounds A5-2, A5-3, and A5-4 in lieu of Compounds 9A, 9B, and 9B.

Compound A5-3: LC-MS (ESI) m/z: 313 [M+H]⁺; ¹H-NMR (CDCl₃, 400 MHz): δ (ppm) 1.45 (t, J=7.2 Hz, 3H), 1.63-1.72 (m, 2H), 1.80-2.02 (m, 6H), 4.49 (q, J=7.2 Hz, 2H), 4.91-4.95 (m, 1H), 7.12-7.19 (m, 2H), 7.76 (d, J=8.8 Hz, 1H), 7.85 (d, J=9.2 Hz, 1H), 7.99 (d, J=10.4 Hz, 1H), 8.45 (s, 1H).

Compound A5-4: LC-MS (ESI) m/z: 297 [M−OH]⁺; ¹H-NMR (CDCl₃, 400 MHz): δ (ppm) 1.21 (t, J=6.8 Hz, 3H), 1.63-1.69 (m, 2H), 1.78-1.86 (m, 2H), 1.88-2.03 (m, 4H), 3.54 (d, J=5.6 Hz, 1H), 4.11-4.31 (m, 2H), 4.87-4.91 (m, 1H), 5.28 (d, J=6.0 Hz, 1H), 7.10-7.13 (m, 2H), 7.45 (d, J=10.4 Hz, 1H), 7.71 (t, J=8.4 Hz, 2H), 7.79 (s, 1H).

Compound A5: LC-MS (m/z) 287 [M+H]⁺

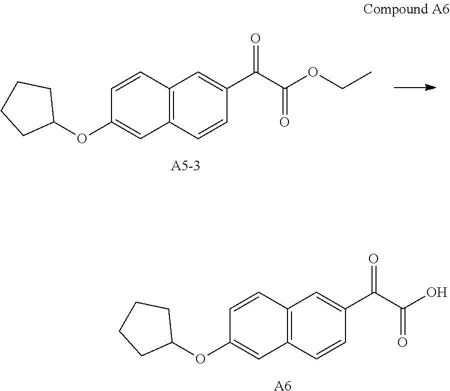

Compound A6 was synthesized by employing the procedure described for Compound 9C using Compound A5-3 in lieu of Compound 9B. LC-MS: (m/z) 285 [M+1]⁺.

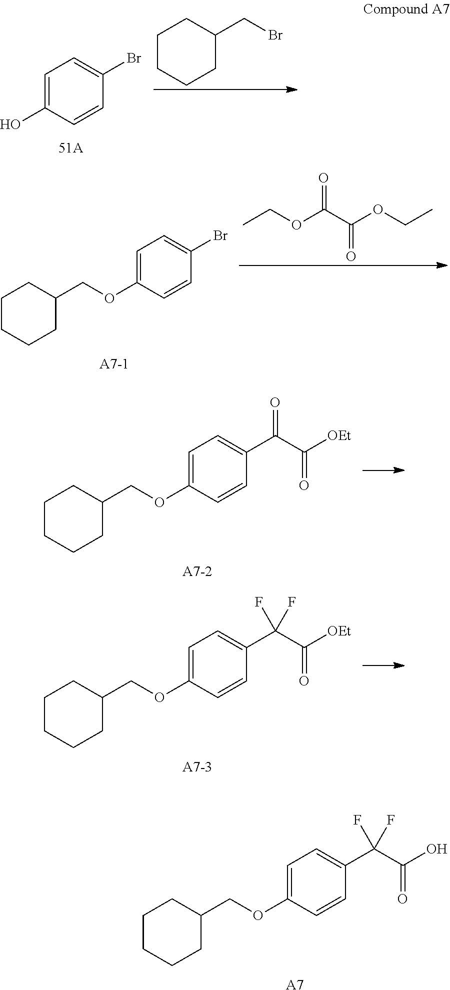

Compounds A7-1, A7-2, A7-3, and A7 were synthesized by employing the procedures described for Compound 4D, Compound 9B, Compound B2-3, and Compound 9C using Compound 51A using K₂CO₃ as base, Compounds A7-1, A7-2, and A7-3 in lieu of Compounds 4C using Cs₂CO₃ as base, Compound 9A, Compound B2-2, and Compound 9B.

Compound A7-1: ¹H-NMR (CDCl₃, 400 MHz): δ (ppm) 0.98-1.08 (m, 2H), 1.17-1.33 (m, 3H), 1.67-1.87 (m, 6H), 3.70 (d, J=6.4 Hz, 2H), 6.74-6.78 (m, 2H), 7.33-7.37 (m, 2H).

Compound A7-2: LC-MS (ESI) m/z: 291 [M+H]⁺.

Compound A7-3: LC-MS (ESI) m/z: 293 [M−F]⁺; ¹H-NMR (CDCl₃, 400 MHz): δ (ppm) 0.85-1.37 (m, 8H), 1.68-1.87 (m, 6H), 3.76 (d, J=6.0 Hz, 2H), 4.28 (q, J=6.8 Hz, 2H), 6.92 (d, J=8.8 Hz, 2H), 7.50 (d, J=8.8 Hz, 2H).

Compound A7: LC-MS (ESI) m/z: 265 [M−F]⁺; ¹H-NMR (CDCl₃, 400 MHz): δ (ppm) 0.99-1.09 (m, 2H), 1.14-1.35 (m, 3H), 1.69-1.87 (m, 6H), 3.77 (d, J=6.4 Hz, 2H), 6.92 (d, J=8.8 Hz, 2H), 7.52 (d, J=8.8 Hz, 2H).

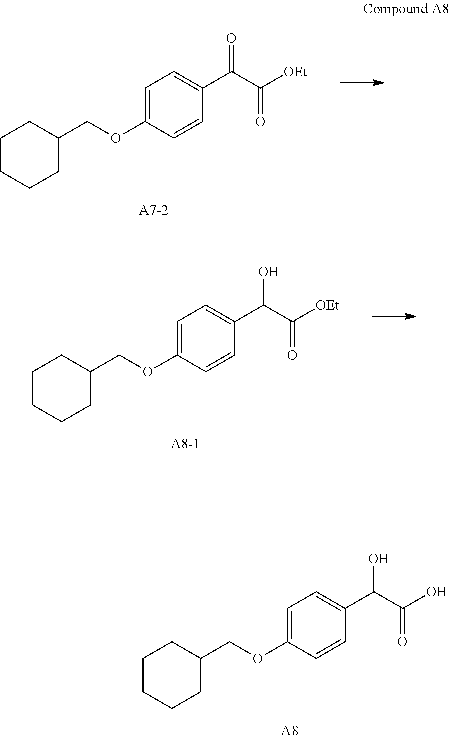 [SMILES: CCOC(=O)C(=O)c1ccc(OCC2CCCCC2)cc1 ; CCOC(=O)C(O)c1ccc(OCC2CCCCC2)cc1 ; O=C(O)C(O)c1ccc(OCC2CCCCC2)cc1 ; "A8"]

To a solution of Compound A7-2 (1 g, 3.4 mmol) in methanol (20 mL) was added NaBH₄ (131 mg, 3.4 mmol) at 0° C. and stirred at 0° C. for 1 hour. The reaction mixture was quenched with 1N HCl (1 mL) and extracted with ethyl acetate (50 mL×2). The combined organic were washed with water (50 mL) and brine (50 mL), dried over anhydrous sodium sulfate, filtered, and concentrated under reduced pressure to provide a residue. The residue was purified by silica gel column chromatography to give Compound A8-1. LC-MS (ESI) m/z: 275 [M−OH]⁺; ¹H-NMR (CDCl₃, 400 MHz): δ (ppm) 1.02-1.05 (m, 2H), 1.20-1.30 (m, 6H), 1.74-1.87 (m, 6H), 3.73 (d, J=6.4 Hz, 2H), 4.16-4.18 (m, 1H), 4.23-4.25 (m, 1H), 5.09 (s, 1H), 6.87 (d, J=8.8 Hz, 2H), 7.30 (d, J=8.8 Hz, 2H).

Compound A8 was synthesized by employing the procedure described for 9C using Compound A8-1 in lieu of Compound 9B. LC-MS (ESI) m/z: 247 [M−OH]⁺; ¹H-NMR (DMSO-d₆, 400 MHz): δ (ppm) 1.0-1.06 (m, 2H), 1.17-1.25 (m, 3H), 1.63-1.80 (m, 6H), 3.75 (d, J=6.4 Hz, 2H), 4.93 (s, 1H), 5.7 (s, 1H), 6.87 (d, J=8.8 Hz, 2H), 7.29 (d, J=8.4 Hz, 2H), 12.5 (s, 1H).

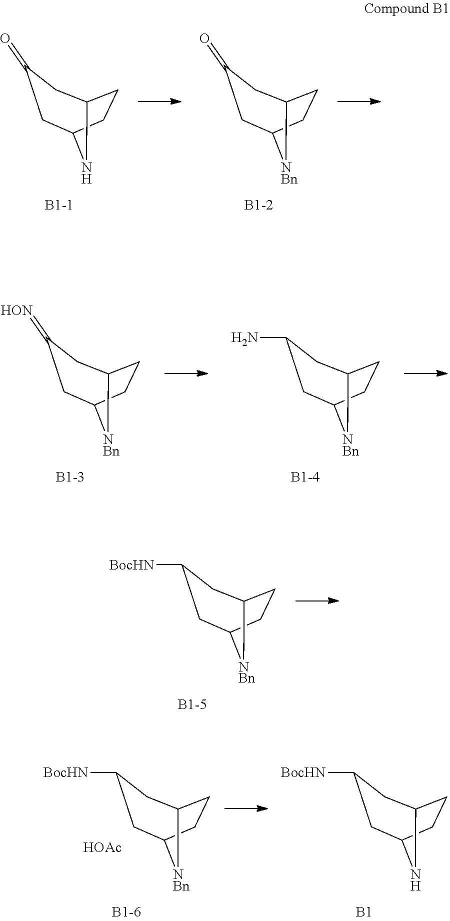

To a solution of BnCl (20.2 g, 160 mmol) in dry THF (400 mL) was added 8-azabicyclo[3.2.1]octan-3-one B1-1 (25.85 g, 160 mmol) and triethylamine (55.2 mL, 400 mmol). The reaction mixture was heated at 70° C. overnight. The reaction mixture was treated with water (200 mL) and extracted with dichloromethane (700 mL×2). The organic extracts were combined, dried over anhydrous sodium sulfate, filtered, and concentrated under reduced pressure to provide a residue. The residue was purified by silica gel column chromatography by silica gel column chromatography to give Compound B1-2. LC-MS (ESI) m/z: 216 [M+H]⁺.

A mixture of hydroxylamine hydrochloride (12.9 g, 181 mmol) in methanol (60 mL) at 0° C. was treated with Na₂CO₃ (13.4 g, 123 mmol) and stirred for 5 minutes. To the mixture was added a solution of Compound B1-2 (32 g, 145 mmol) in methanol (420 mL) and stirred under nitrogen at 20° C. for 16 hours. The reaction mixture was concentrated under reduced pressure. The residue was treated with brine (200 mL) and extracted with dichloromethane (700 mL×2). The organic extracts were combined, dried over anhydrous sodium sulfate, filtered, concentrated, and washed with ethyl acetate to afford Compound B1-3. LC-MS (ESI) m/z: 231 [M+H]⁺; ¹H-NMR (CDCl₃, 400 MHz): δ (ppm) 1.49-1.64 (m, 2H), 1.96-1.98 (m, 2H), 2.11-2.15 (m, 1H), 2.21-2.26 (m, 1H), 2.57-2.62 (m, 1H), 2.96-3.00 (m, 1H), 3.33-3.36 (m, 2H), 3.61-3.69 (s, 2H), 7.23-7.27 (m, 1H), 7.30-7.34 (m, 2H), 7.35-7.41 (m, 2H), 9.26 (s, 1H).

A solution of Compound B1-3 (23 g, 100 mmol) in absolute butan-1-ol (500 mL) was heated at 120° C. To the solution was added sodium metal (28 g, 1.2 mol) in several small pieces over 2.5 hours. The reaction mixture was heated at 120° C. for 4 hours. The reaction mixture was cooled, treated with water (500 mL) carefully, and extracted with ether (300 mL×3). The organic extracts were combined, washed with brine (60 mL), dried over anhydrous sodium sulfate, filtered, and evaporated to furnish Compound B1-4. LC-MS (ESI) m/z: 217 [M+H]⁺.

To a solution of Compound B1-4 (4.95 g, 22.9 mmol) in dry dichloromethane (70 mL) was added Boc₂O (5.55 g, 25.2 mmol) and triethylamine (3.8 mL, 27.5 mmol). The reaction mixture was heated at reflux overnight, diluted with dichloromethane (150 mL), and washed with 5% sodium bicarbonate (70 mL) and brine (70 mL). The organic layer was dried over anhydrous sodium sulfate, filtered, and concentrated under reduced pressure a residue. The residue was purified by silica gel column chromatography to furnish Compound B1-5. LC-MS (ESI) m/z: 317 [M+H]⁺; ¹H-NMR (CDCl₃, 400 MHz): δ (ppm) 1.43 (s, 9H), 1.44-1.53 (m, 2H), 1.67-1.70 (m, 2H), 1.78-1.82 (m, 2H), 2.00-2.03 (m, 2H), 3.19-3.21 (m, 2H), 3.51 (s, 2H), 3.82 (s, 1H), 4.35 (s, 1H), 7.23-7.37 (m, 5H).

To a solution of Compound B1-5 (8.4 g, 26.6 mmol) and glacial acetic acid (1 eq.) in ethanol (350 mL) was added 10% Pd/C (1 g) at 22° C. The solution was stirred under H₂ (4.5 atm) at 22° C. for 24 hours. The reaction mixture was filtered and concentrated to give Compound B1-6. LC-MS (ESI) m/z: 227 [M+H]⁺; ¹H-NMR (CD₃OD, 400 MHz): δ (ppm) 1.44 (s, 9H), 1.73-1.77 (m, 2H), 1.99-2.13 (m, 6H), 3.81-3.95 (m, 1H), 4.03 (s, 2H), 4.97 (m, 2H), 5.12-5.13 (m, 1H), 6.41-6.43 (m, 1H), 7.14-7.29 (m, 2H).

To a solution of Compound B1-6 (5 g, 17.5 mmol) in dichloromethane (150 mL) was added 10% NaOH (20 mL) at 10° C. The reaction mixture was stirred at 22° C. for 0.5 hour and extracted with dichloromethane (100 mL×3). The combined organic layers was washed with brine (60 mL), dried over anhydrous sodium sulfate, filtered, and concentrated to afford Compound B1. LC-MS (ESI) m/z: 227 [M+H]⁺; ¹H-NMR (CDCl₃, 400 MHz): δ (ppm) 1.44 (s, 9H), 1.74-1.80 (m, 5H), 1.90-1.95 (m, 2H), 3.55 (s, 2H), 3.81-3.85 (brs, 1H), 4.36-4.40 (m, 1H).

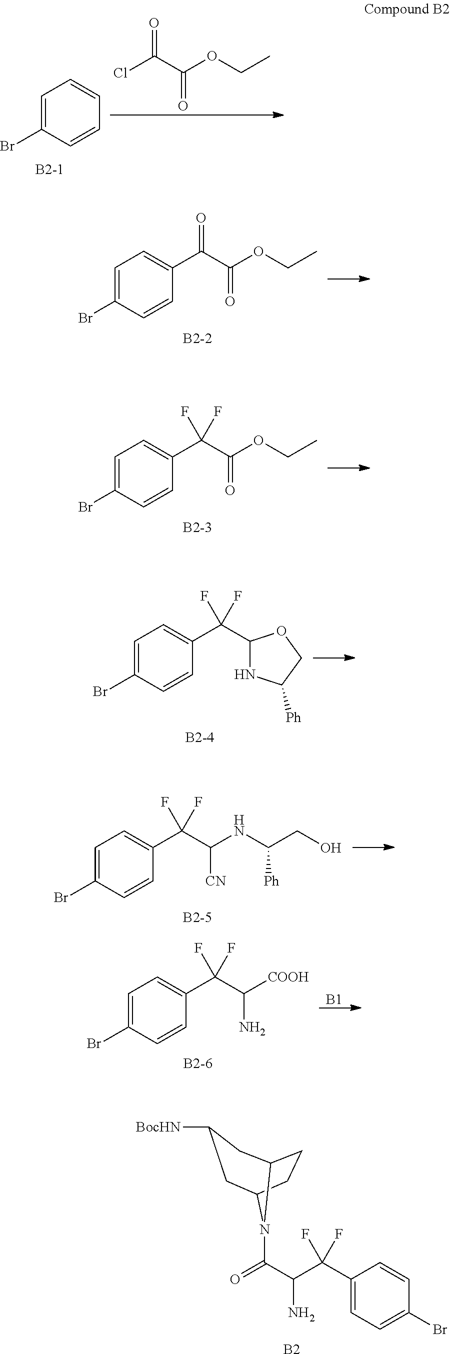

To a suspension of aluminum trichloride (2.2 g, 16 mmol) and ethyl chloroacetate (1.9 mL, 16 mmol) was dropped neat bromobenzene B2-1 (1.5 mL, 14 mmol) and stirred at room temperature overnight. The reaction mixture was quenched by carefully adding a solution of ice water (100 mL) and saturated sodium bicarbonate solution (100 mL). The aqueous layer was extracted with dichloromethane (50 mL×3). The combined organic layers were washed with brine (200 mL), dried over anhydrous sodium sulfate, filtered, and concentrated to provide a residue. The residue was purified by silica gel column chromatography by silica gel column chromatography to furnish Compound B2-2. ¹H-NMR (CDCl₃, 400 MHz): δ (ppm) 1.35 (t, J=6.8 Hz, 3H), 4.37 (q, J=7.2 Hz, 2H), 7.56 (d, J=8.8 Hz, 2H), 7.82 (d, J=8.8 Hz, 2H).

To a solution of Compound B2-2 (2.56 g, 10 mmol) in dichloromethane (350 mL) was added DAST (4 mL) at room temperature. The reaction mixture was stirred at room temperature for 14 hours. The reaction mixture was poured into ice water (50 mL) and extracted with dichloromethane (50 mL×2). The combined organic layers were washed with brine (200 mL), dried over anhydrous sodium sulfate, filtered, and concentrated to provide a residue. The residue was purified by silica gel column chromatography by silica gel column chromatography to furnish Compound B2-3. LC-MS (ESI) m/z: 279 [M+H]⁺; ¹H-NMR (CDCl₃, 400 MHz): δ (ppm) 1.30 (t, J=6.8 Hz, 3H), 4.31 (q, J=7.2 Hz, 2H), 7.48 (d, J=8.8 Hz, 2H), 7.59 (d, J=8.8 Hz, 2H).

To a solution of Compound B2-3 (1.39 g, 5 mmol) in methanol (5 mL) at −60° C. was added NaBH₄ (190 mg, 5 mmol) in several small portions. The reaction mixture was stirred at −50° C. for 1 hour, quenched with a diluted aqueous HCl solution (1 N, 10 mL), and extracted with ethyl acetate (50 mL×3). The combined organic extracts were washed with brine (50 mL), dried over anhydrous sodium sulfate, filtered, and concentrated under reduced pressure to provide a residue. The residue was added to a solution of (S)-phenylglycinol (685 mg, 5 mmol) and PPTS (20 mg) in toluene (20 mL). The mixture was refluxed to remove water by azeotropic distillation until no water remained. The organic solvent was removed under reduced pressure to provide a residue. The residue was purified with by silica gel column chromatography to furnish Compound B2-4. LC-MS (ESI) m/z: 354 [M+H]⁺; ¹H-NMR (CDCl₃, 400 MHz): δ (ppm) 2.57 (brs, 1H), 3.35-3.65 (m, 1H), 3.95-4.47 (m, 2H), 5.09-5.20 (m, 1H), 7.21-7.35 (m, 5H), 7.48 (d, J=8.8 Hz, 2H), 7.59 (d, J=8.8 Hz, 2H).

To a solution of Compound B2-4 (1.76 g, 5 mmol) in dichloromethane (100 mL) was added cyanotrimethylsilane (990 mg, 10 mmol) and BF₃-OEt₂ (1.42 g, 10 mmol) at −78° C. The reaction mixture was stirred at room temperature overnight and poured into a saturated aqueous solution of sodium bicarbonate (25 mL). The aqueous layer was extracted with dichloromethane (25 mL×3). The combined organic layers were washed with brine (50 mL), dried over anhydrous sodium sulfate, filtered, and concentrated to provide a residue. The residue was purified with by silica gel column chromatography to furnish Compound B2-5. LC-MS (ESI) m/z: 381 [M+H]⁺; ¹H-NMR (CDCl₃, 400 MHz): δ (ppm) 3.46 (t, J=10.4 Hz, 1H). 3.74-3.79 (m, 2H), 4.03-4.06 (m, 1H), 7.00-7.02 (d, J=7.6 Hz, 2H), 7.22-7.28 (m, 3H), 7.33-7.35 (m, 2H), 7.59-7.62 (d, J=7.6 Hz, 2H).

To a solution of Compound B2-5 (1.9 g, 5 mmol) in methanol (50 mL) and dichloromethane (100 mL) was added lead (IV) acetate (3.32 g, 7.5 mmol) at 0° C. After the completion of the reaction, the reaction mixture was poured into an aqueous phosphate buffer solution (0.2 M NaH₂PO₄/0.2 M Na₂HPO₄; 1/1; v/v, 150 mL) at room temperature and filtered with Celite. The aqueous layer was extracted with dichloromethane (150 mL×3). The combined organic extracts were washed with brine (100 mL), dried over anhydrous sodium sulfate, filtered, and concentrated to provide a residue. The residue was treated with concentrated HCl (5 mL) and stirred at 110° C. overnight. After cooling the reaction mixture to room temperature, the reaction mixture was extracted with ether (300 mL) and the aqueous solution was concentrated to give a crude product, which was purified with reverse phase chromatography to furnish Compound B2-6. LC-MS (ESI) m/z: 280 [M+H]⁺; ¹H-NMR (CDCl₃, 400 MHz): δ (ppm) 4.56-4.84 (m, 1H), 7.46-7.52 (m, 2H), 7.74-7.86 (m, 2H), 9.25 (br s, 1H).

To a mixture of Compound B2-6 (155 mg, 0.5 mmol), Compound B1 (452 mg, 1 mmol), EDCI (286 mg, 1.5 mmol), and HOBt (202 mg, 1.5 mmol) in dichloromethane (10 mL) was added N,N-diisopropylethylamine (1 mL). The reaction mixture was stirred at 25° C. overnight and diluted with dichloromethane (30 mL). The organic layer was washed with water (40 mL) and brine (40 mL), dried over anhydrous sodium sulfate, filtered, and concentrated to provide a residue. The residue was purified with reverse phase chromatography to furnish Compound B2. LC-MS (ESI) m/z: 488 [M+H]⁺; ¹H-NMR (CD₃OD, 400 MHz): δ (ppm) 1.31-1.36 (m, 9H), 1.51-2.07 (m, 8H), 3.76-3.78 (m, 1H), 3.97-4.09 (m, 1H), 4.25-4.57 (m, 2H), 7.29-7.37 (m, 2H), 7.53-7.55 (m, 2H).

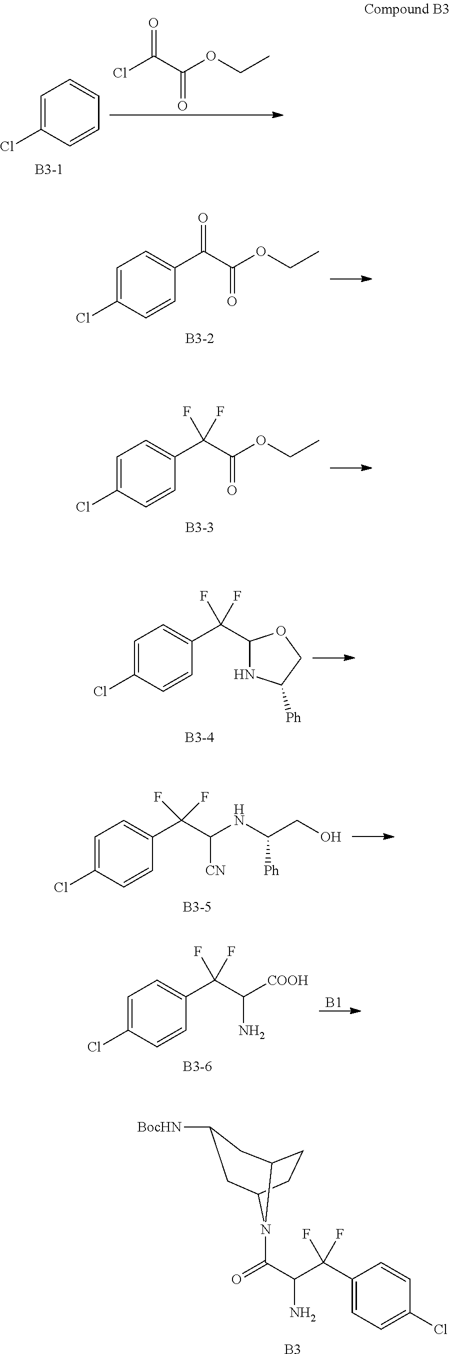

Compounds B3-2, B3-3, B3-4, B3-5, B3-6, and B3 were synthesized by employing the procedures described for Compounds B2-2, B2-3, B2-4, B2-5, B2-6, and B2 using Compounds B3-1, B3-2, B3-3, B3-4, B3-5, and B3-6 in lieu of Compounds B2-1, B2-2, B2-3, B2-4, B2-5, and B2-6.

Compound B3-2: ¹H-NMR (CDCl₃, 400 MHz): δ (ppm) 1.35 (t, J=6.8 Hz, 3H), 4.37 (q, J=7.2 Hz, 2H), 7.41 (d, J=8.8 Hz, 2H), 7.91 (d, J=8.8 Hz, 2H).

Compound B3-3: ¹H-NMR (CDCl₃, 400 MHz): δ (ppm) 1.23 (t, J=7.2 Hz, 3H), 4.23 (q, J=7.2 Hz, 2H), 7.36 (d, J=8.4 Hz, 2H), 7.48 (d, J=8.4 Hz, 2H).

Compound B3-4: LC-MS (ESI) m/z: 310 [M+H]⁺; ¹H-NMR (CDCl₃, 400 MHz): δ (ppm) 2.52 (brs, 1H), 3.25-3.57 (m, 1H), 3.85-4.41 (m, 2H), 5.02-5.14 (m, 1H), 7.14-7.28 (m, 5H), 7.34 (d, J=8.4 Hz, 2H), 7.49 (d, J=8.4 Hz, 2H).

Compound B3-5: LC-MS (ESI) m/z: 337 [M+H]⁺; ¹H-NMR (CDCl₃, 400 MHz): δ (ppm) 2.54 (d, J=13.2 Hz, 1H), 2.41 (t, J=10 Hz, 1H), 3.65-3.74 (m, 2H), 3.96-3.99 (m, 1H), 6.94 (d, J=6.4 Hz, 2H), 7.15-7.21 (m, 3H), 7.33-7.40 (m, 4H).

Compound B3-6: LC-MS (ESI) m/z: 236 [M+H]⁺; ¹H-NMR (CDCl₃, 400 MHz): δ (ppm) 4.43-4.48 (m, 1H), 7.56 (s, 4H), 8.62 (brs, 1H).

Compound B3 was used in the next step without further purification. LC-MS (ESI) m/z: 444 [M+H]⁺.

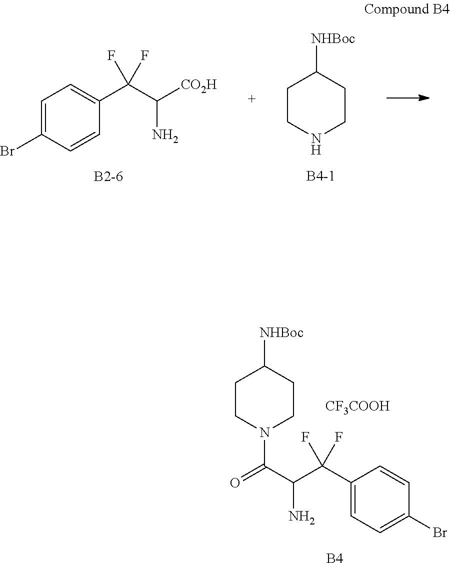

To a solution of Compound B2-6 (130 mg, 0.46 mmol) in dichloromethane (20 mL) was added tert-butyl piperidin-4-ylcarbamate, B4-1, (93 mg, 0.46 mmol), HATU (265 mg, 0.69 mmol), and DIPEA (120 mg, 0.93 mmol) and stirred at room temperature for 4 hours. The reaction mixture was quenched with water (20 mL) and extracted with dichloromethane (20 mL×3). The combined organic extracts were washed with brine (30 mL), dried over anhydrous sodium sulfate, filtered, and concentrated to provide a residue. The residue was purified with reverse phase chromatography to furnish Compound B4. LC-MS (ESI) m/z: 462 [M+H]⁺; ¹H-NMR (CDCl₃, 400 MHz): δ (ppm) 0.98-1.25 (m, 3H), 1.42 (s, 9H), 1.86-1.92 (m, 3H), 2.69-3.13 (m, 2H), 3.62 (s, 1H), 3.81 (dd, J=35.2, 15.2 Hz, 1H), 4.29 (t, J=10 Hz, 1H), 4.42-4.60 (m, 2H), 7.32 (dd, J=12.8, 8.3 Hz, 1H), 7.56 (t, J=7.4 Hz, 1H).

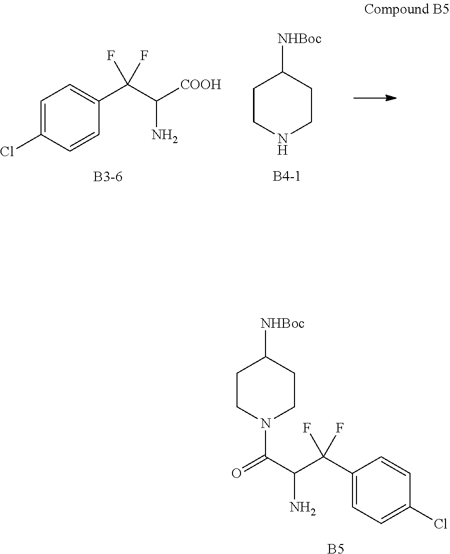

Compound B5 was synthesized by employing the procedure described for Compound B2 using Compound B3-6 and B4-1 in lieu of Compound B2-6 and B1. LC-MS (ESI) m/z: 418 [M+H]⁺.

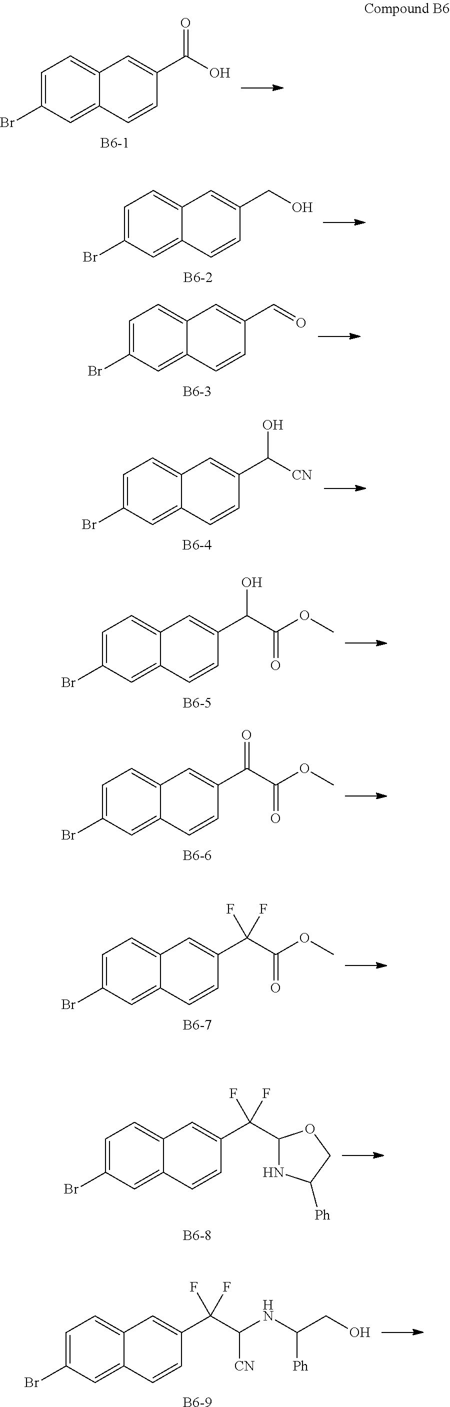

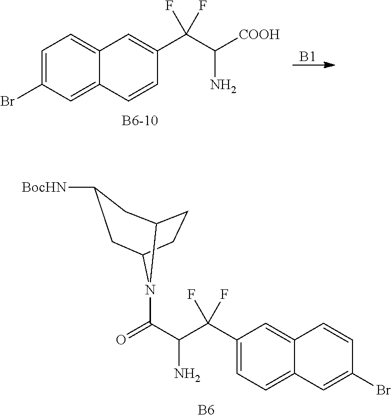

To a solution of compound B6-1 (2.51 g, 10 mmol) in THF (60 mL) was added a solution of BH₃-THF in THF (1 M, 15 mL) at 0° C. The reaction mixture was stirred at room temperature overnight, quenched with water (30 mL), and extracted with ethyl acetate (100 mL×2). The combined organic extracts were washed with aqueous sodium hydroxide solution (1 N, 100 mL) and brine (100 mL), dried over anhydrous sodium sulfate, filtered, and concentrated to give Compound B6-2. LC-MS (ESI) m/z: 219 [M−OH]⁺; ¹H-NMR (CDCl₃, 400 MHz): δ (ppm) 1.98 (s, 1H), 4.84 (s, 2H), 7.48-7.56 (m, 2H), 7.68-7.77 (m, 3H), 7.99 (s, 1H).

To a solution of compound B6-2 (4 g, 16.9 mmol) in dichloromethane (60 mL) was added Dess-Martin periodinane (10.8 g, 25.4 mmol) at 0° C. The reaction mixture was stirred at room temperature overnight, quenched with saturated Na₂S203 solution (60 mL), and diluted with ethyl acetate (400 mL). The organic layer was washed with water (200 mL) and brine (200 mL), dried over anhydrous sodium sulfate, filtered, and concentrated to provide a residue. The residue was purified by silica gel column chromatography by silica gel column chromatography to afford Compound B6-3. LC-MS (ESI) m/z: non-ionizable compound under routine conditions used; ¹H-NMR (CDCl₃, 400 MHz): δ (ppm) 7.66-7.68 (m, 1H), 7.84-7.89 (m, 2H), 7.97-7.98 (m, 1H), 8.08 (s, 1H), 8.32 (s, 1H), 10.15 (s, 1H).

A mixture of Compound B6-3 (2 g, 8.5 mmol), sodium cyanide (1.3 mg, 25.6 mmol), and AcOH (1.5 g, 25.6 mmol) in methanol (50 mL) was stirred at 25° C. overnight. The reaction mixture was diluted with ethyl acetate (200 mL), washed with water (100 mL) and brine (100 mL), dried over anhydrous sodium sulfate, filtered, and concentrated to yield Compound B6-4, which was directly used for the next step without further purification. ¹H-NMR (DMSO-d₆, 400 MHz): δ (ppm) 5.96 (d, J=6.4 Hz, 1H), 7.26 (d, J=6.8 Hz, 1H), 7.65-7.71 (m, 2H), 7.99-8.02 (m, 2H), 8.08 (s, 1H), 8.27 (d, J=1.6 Hz, 1H).

Compounds B6-5, B6-6, B6-7, B6-8, B6-9, B6-10, and B6 were synthesized by employing the procedures described for Compound 45G, Compounds B6-3, B2-3, B2-4, B2-5, B2-6, and B2 using Compounds B6-4 using MeOH as solvent, B6-5, B6-6, B6-7, B6-8, B6-9, and B6-10 in lieu of Compound 45F using EtOH as solvent, Compounds B6-2, B2-2, B2-3, B2-4, B2-5, and B2-6.

Compound B6-5: LC-MS (ESI) m/z: 295 [M+H]⁺; ¹H-NMR (DMSO-d₆, 400 MHz): δ (ppm) 3.63 (s, 3H), 5.34 (d, J=4.8 Hz, 1H), 6.31 (d, J=5.2 Hz, 1H), 7.59-7.66 (m, 2H), 7.90-7.94 (m, 2H), 7.99 (s, 1H), 8.21 (d, J=1.2 Hz, 1H).

Compound B6-6: LC-MS (ESI) m/z: 293 [M+H]⁺; ¹H-NMR (CDCl₃, 400 MHz): δ (ppm) 4.04 (s, 3H), 7.67-7.68 (m, 1H), 7.84-7.86 (m, 2H), 8.07 (s, 2H), 8.56 (s, 1H).

Compound B6-7: LC-MS (ESI) m/z: 315 [M+H]⁺; ¹H-NMR (CDCl₃, 400 MHz): δ (ppm) 3.86 (s, 3H), 7.62-7.69 (m, 2H), 7.77-7.84 (m, 2H), 8.05-8.09 (m, 2H).

Compound B6-8: LC-MS (ESI) m/z: 404 [M+H]⁺; ¹H-NMR (CDCl₃, 400 MHz): δ (ppm) 2.62 (brs, 1H), 3.29-3.66 (m, 0.5H), 3.62-3.66 (m, 0.5H), 3.96 (s, 0.5H), 4.12-4.16 (m, 1H), 4.45 (s, 0.5H), 5.22-5.30 (m, 1H), 7.19-7.32 (m, 5H), 7.60-7.62 (m, 1H), 7.72-7.83 (m, 3H), 8.05-8.10 (m, 2H).

Compound B6-9: LC-MS (ESI) m/z: 431 [M+H]⁺; ¹H-NMR (CDCl₃, 400 MHz): δ (ppm) 1.74-1.77 (m, 1H), 2.66-2.69 (m, 1H), 3.44-3.46 (m, 1H), 3.70-3.74 (m, 1H), 3.85-3.95 (m, 1H), 4.04-4.07 (m, 1H), 6.94-7.13 (m, 3H), 7.21-7.34 (m, 2H), 7.52-7.55 (m, 1H), 7.65-7.67 (m, 1H), 7.76-7.84 (m, 2H), 7.95 (s, 1H), 8.09 (s, 1H).

Compound B6-10: LC-MS (ESI) m/z: 330 [M+H]⁺; ¹H-NMR (DMSO-d₆, 400 MHz): δ (ppm) 4.21-4.28 (m, 1H), 7.67-7.73 (m, 2H), 7.98-8.02 (m, 2H), 8.14 (s, 1H), 8.29 (d, J=1.6 Hz, 1H).

Compound B6: LC-MS (ESI) m/z: 538 [M+H]⁺; ¹H-NMR (DMSO-d₆, 400 MHz): δ (ppm) 0.84-0.86 (m, 1H), 1.34 (s, 9H), 1.45-1.49 (m, 2H), 1.64-1.76 (m, 3H), 2.05-2.20 (m, 2H), 3.52-3.78 (m, 1H), 4.18-4.55 (m, 3H), 6.69-6.75 (m, 1H), 7.63-7.73 (m, 2H), 7.98-8.08 (m, 3H), 8.29 (s, 1H).

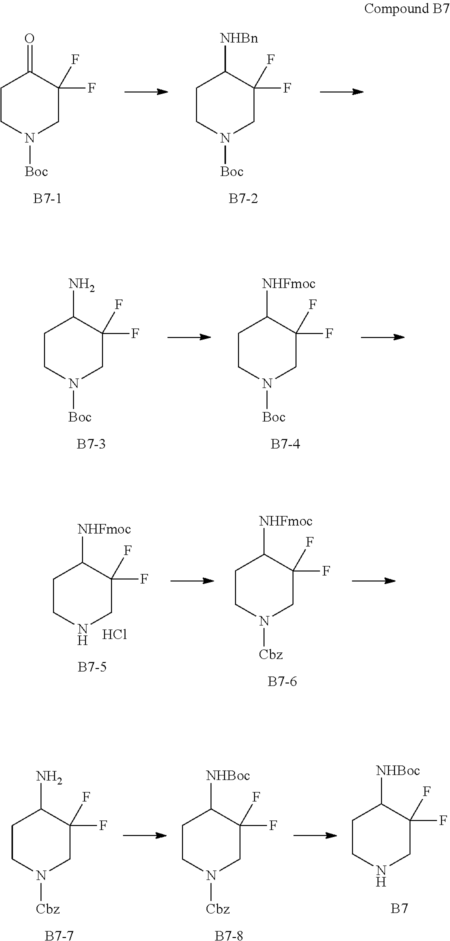

A solution of tert-butyl 3,3-difluoro-4-oxopiperidine-1-carboxylate B7-1 (2.00 g, 8.51 mmol) and phenylmethanamine (1.82 g, 17.02 mmol) in dichloromethane (20 mL) was stirred at room temperature for 3 hours. To the solution was added NaBH(OAc)₃ (3.61 g, 17.02 mmol) and stirred at room temperature for 16 hours. The reaction mixture was diluted with ethyl acetate (160 mL), washed with water (120 mL×3) and brine (100 mL), dried over anhydrous sodium sulfate, filtered, and concentrated under reduced pressure to provide a residue. The residue was purified by silica gel column chromatography by silica gel column chromatography to yield Compound B7-2. LC-MS (ESI) m/z: 327 [M+H]⁺.

A mixture of Compound B7-2 (2.00 g, 6.13 mmol) and 10% Pd/C (1.00 g) in methanol (30 mL) was stirred under hydrogen (1 atmosphere) at 20° C. for 16 hours. The mixture was filtered through Celite to provide a filtrate. The filtrate was concentrated to give a crude Compound B7-3. LC-MS (ESI) m/z: 259 [M+Na]⁺; ¹H-NMR (CDCl₃, 400 MHz): δ (ppm) 1.49 (s, 9H), 1.77-1.94 (m, 2H), 2.91-3.08 (m, 1H), 3.45-3.73 (m, 2H), 3.94-4.17 (m, 2H).

To a solution of Compound B7-3 (2.30 g, 9.74 mmol) and NaHCO₃ (1.64 g, 19.48 mmol) in 1,4-dioxane (10 mL) and H₂O (2 mL) was added FmocCl (3.03 g, 11.69 mmol) in several small portions at 0° C. The reaction mixture was stirred at room temperature for 3 hours and diluted with ethyl acetate (160 mL). The organic layer was washed with water (120 mL×3) and brine (100 mL), dried over anhydrous sodium sulfate, filtered, and concentrated under reduced pressure to provide a residue. The residue was purified by silica gel column chromatography by silica gel column chromatography to yield Compound B7-4. LC-MS (ESI) m/z: 459 [M+H]⁺; ¹H-NMR (CDCl₃, 400 MHz): δ (ppm) 1.47 (s, 9H), 1.64-1.96 (m, 2H), 2.84-3.07 (m, 1H), 3.45-3.73 (m, 2H), 3.93-4.08 (m, 1H), 4.19-4.27 (m, 1H), 4.38-4.48 (m, 2H), 5.03 (s, 1H), 7.30-7.43 (m, 4H), 7.58 (d, J=7.2 Hz, 2H), 7.77 (d, J=7.6 Hz, 2H).

A mixture of Compound B7-4 (2.30 g, 5.02 mmol), HCl solution in 1,4-dioxane (4 M, 3 mL), and dichloromethane (10 mL) was stirred at 20° C. for 16 hours. The reaction mixture was filtered. The cake was washed with diethyl ether (20 mL) and dried under vacuum to afford Compound B7-5. LC-MS (ESI) m/z: 359 [M+H]⁺; ¹H-NMR (DMSO-d₆, 400 MHz): δ (ppm) 1.88-2.02 (m, 2H), 3.09-3.22 (m, 1H), 3.51-3.74 (m, 3H), 4.24-4.40 (m, 3H), 7.32 (t, J=7.2 Hz, 2H), 7.43 (t, J=7.2 Hz, 2H), 7.71-7.75 (m, 2H), 7.90 (d, J=7.2 Hz, 3H), 9.33 (s, 2H).

To a mixture of Compound B7-5 (2.00 g, 5.08 mmol) and NaHCO₃ (1.28 g, 15.24 mmol) in THF (30 mL) and H₂O (10 mL) was added CbzCl (954 mg, 5.58 mmol) in several small portions at 0° C. The reaction mixture was stirred at room temperature for 3 hours and diluted with ethyl acetate (160 mL). The organic layer was washed with water (120 mL×3) and brine (100 mL), dried over anhydrous sodium sulfate, filtered, and concentrated to provide a residue. The residue was purified by silica gel column chromatography by silica gel column chromatography to yield Compound B7-6. ¹H-NMR (DMSO-d₆, 400 MHz): δ (ppm) 1.61-1.86 (m, 2H), 3.05-3.67 (m, 4H), 3.82-4.16 (m, 4H), 5.10 (d, J=3.6 Hz, 2H), 7.31-7.44 (m, 9H), 7.71-7.90 (m, 4H).

A mixture of Compound B7-6 (2.20 g, 4.47 mmol) and piperidine (5 mL) in dichloromethane (20 mL) was stirred at 20° C. for 16 hours. The reaction mixture was diluted with ethyl acetate (160 mL), washed with water (120 mL×3) and brine (100 mL), dried over anhydrous sodium sulfate, filtered, and concentrated under reduced pressure to provide a residue. The residue was purified by silica gel column chromatography by silica gel column chromatography to yield Compound B7-7. LC-MS (ESI) m/z: 271 [M+H]⁺; ¹H-NMR (CDCl₃, 400 MHz): δ (ppm) 1.33-1.59 (m, 1H), 1.92-2.01 (m, 1H), 2.99-3.27 (m, 3H), 4.00-4.13 (m, 2H), 5.16 (s, 2H), 7.31-7.39 (m, 5H).

To a solution of Compound B7-7 (600 mg, 2.22 mmol) and K₂CO₃ (307 mg, 4.44 mmol) in 1,4-dioxane (10 mL) and H₂O (3 mL) was added (Boc)₂O (968 mg, 4.44 mmol) at room temperature. The reaction mixture was stirred at room temperature for 16 hours and diluted with ethyl acetate (160 mL). The organic layer was washed with water (120 mL×3) and brine (100 mL), dried over anhydrous sodium sulfate, filtered, and concentrated under reduced pressure to provide a residue. The residue was purified by silica gel column chromatography by silica gel column chromatography to yield Compound B7-8. LC-MS (ESI) m/z: 371 [M+H]⁺; ¹H-NMR (DMSO-d₆, 400 MHz): δ (ppm) 1.39 (s, 9H), 1.52-1.61 (m, 1H), 1.72-1.77 (m, 1H), 3.02-3.20 (m, 1H), 3.37-3.54 (m, 1H), 3.88-4.16 (m, 3H), 5.09 (s, 2H), 7.23 (d, J=9.2 Hz, 1H), 7.31-7.39 (m, 5H).

A mixture of Compound B7-8 (700 mg, 1.89 mmol) and 10% Pd/C (300 mg) in methanol (20 mL) was stirred under hydrogen (1 atm) at 20° C. for 16 hours. The reaction mixture was filtered through Celite and the filtrate was concentrated under reduced pressure to provide Compound B7. LC-MS (ESI) m/z: 237 [M+H]⁺; ¹H-NMR (CDCl₃, 400 MHz): δ (ppm) 1.46 (s, 9H), 1.49-1.56 (m, 2H), 2.00-2.04 (m, 1H), 2.63-2.87 (m, 2H), 3.05-3.08 (m, 1H), 3.21-3.28 (m, 1H), 3.95-4.05 (m, 1H), 4.76-4.87 (m, 1H).

### Example 1

### Synthesis of N-((2R)-3-(3-amino-8-azabicyclo[3.2.1]octan-8-yl)-1-(4-bromophenyl)-1,1-difluoro-3-oxopropan-2-yl)-6-isopropoxynaphthalene-2-sulfonamide (Compound 1-1), and N-((2S)-3-(3-amino-8-azabicyclo[3.2.1]octan-8-yl)-1-(4-bromophenyl)-1,1-difluoro-3-oxopropan-2-yl)-6-isopropoxynaphthalene-2-sulfonamide (Compound 1-2)

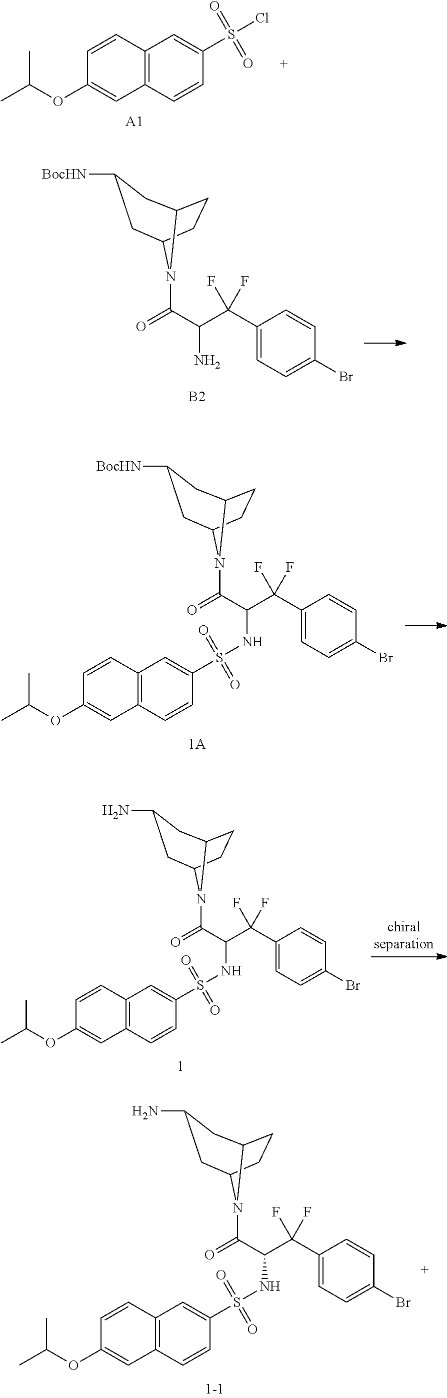

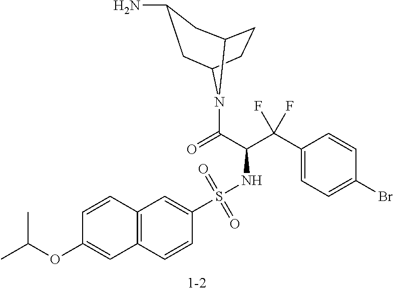 [SMILES: CC(C)Oc1ccc2cc(S(=O)(=O)N[C@@H](C(=O)N3C4CCC3CC(N)C4)C(F)(F)c3ccc(Br)cc3)ccc2c1]

To a mixture of DIPEA (3 mL) and Compound B2 (4.9 g, 10 mmol) in dichloromathane (40 mL) was added Compound A1 (2.8 g, 10 mmol). The reaction mixture was stirred at room temperature overnight. The reaction mixture was purified by silica gel column chromatography to furnish Compound 1A. LC-MS (ESI) m/z: 736 [M+H]⁺.

A solution of Compound 1A (74 mg, 0.1 mmol) in a solution of HCl in 1,4-dioxane (4.0 M, 5 mL) was stirred at room temperature for 3 hours. The reaction mixture was evaporated under reduced pressure and purified with preparative HPLC to give Compound 1, which was separated with chiral HPLC (EtOH contained 0.5% DEA; OZ-H (250×4.6 mm, 5 μm) to provide Compound 1-1 and Compound 1-2.

Compound 1-1: LC-MS (ESI) m/z: 636 [M+H]⁺; ¹H-NMR (MeOD, 400 MHz) δ (ppm) 1.19-1.32 (m, 7H), 1.48-2.21 (m, 7H), 3.46-3.59 (m, 1H), 4.25-4.43 (m, 1H), 4.55-4.74 (m, 3H), 7.08 (s, 2H), 7.16 (m, 1H), 7.23 (m, 1H), 7.35-7.46 (m, 3H), 7.66-7.75 (m, 2H), 7.98-8.07 (m, 1H). Chiral separation condition: MeOH contained 0.1% DEA; AS-H (4.6×250 mm, 5 μm); retention time: 5.4 minute.

Compound 1-2: LC-MS (ESI) m/z: 636 [M+H]⁺; ¹H-NMR (MeOD, 400 MHz) δ (ppm) 1.29-1.34 (m, 7H), 1.48-2.10 (m, 7H), 3.45-3.64 (m, 1H), 4.26-4.44 (m, 1H), 4.55-4.75 (m, 3H), 7.08 (s, 2H), 7.16 (m, 1H), 7.23 (m, 1H), 7.35-7.46 (m, 3H), 7.66-7.75 (m, 2H), 7.98-8.07 (m, 1H). Chiral separation condition: MeOH contained 0.1% DEA; AS-H (4.6×250 mm, 5 μm); retention time: 6.75 minute.

### Example 2

### Synthesis of N-((2R)-3-(3-amino-8-azabicyclo[3.2.1]octan-8-yl)-1-(4-bromophenyl)-1,1-difluoro-3-oxopropan-2-yl)-4-(2-ethylbutoxy)benzenesulfonamide (Compound 2-1), and N-((2S)-3-(3-amino-8-azabicyclo[3.2.1]octan-8-yl)-1-(4-bromophenyl)-1,1-difluoro-3-oxopropan-2-yl)-4-(2-ethylbutoxy)benzenesulfonamide (Compound 2-2)

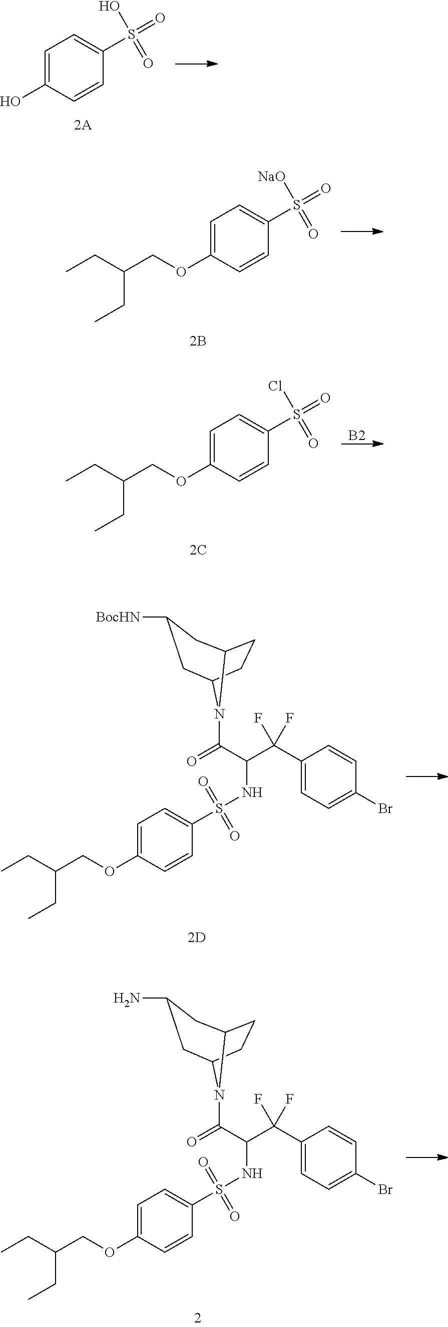

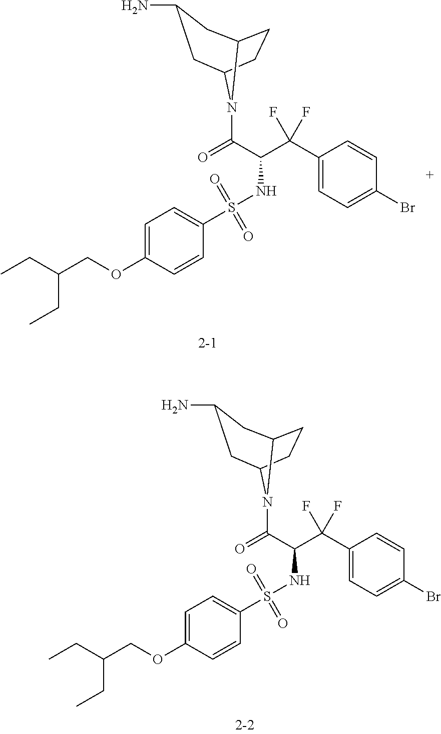 [SMILES: CCC(CC)COc1ccc(S(=O)(=O)N[C@H](C(=O)N2C3CCC2CC(N)C3)C(F)(F)c2ccc(Br)cc2)cc1 ; CCC(CC)COc1ccc(S(=O)(=O)N[C@@H](C(=O)N2C3CCC2CC(N)C3)C(F)(F)c2ccc(Br)cc2)cc1]

A mixture of Compound 2A (1.7 g, 10 mmol), 3-(bromomethyl)pentane (1.7 g, 10 mmol), and NaOH (0.8 g, 20 mmol) in water (20 mL) and EtOH (40 mL) was stirred at 100° C. overnight. The reaction mixture was cooled down and concentrated. To the residue was added EtOH (50 mL) and filtered to afford Compound. ¹H-NMR (DMSO-d₆, 400 MHz): δ (ppm) 0.89 (t, J=7.4 Hz, 6H), 1.40-1.42 (m, 4H), 1.62-1.63 (m, 1H), 3.85 (d, J=6.0 Hz, 2H), 6.85 (d, J=8.4 Hz, 2H), 7.51 (d, J=8.8 Hz, 2H).

Compound 2C was synthesized by employing the procedure described for Compound A1 using Compound 2B in lieu of Compound A1-3. ¹H-NMR (CDCl₃, 400 MHz): δ (ppm) 0.94 (t, J=7.4 Hz, 6H), 1.48-1.49 (m, 4H), 1.71 (m, 1H), 3.95 (d, J=5.6 Hz, 2H), 7.03 (d, J=8.8 Hz, 2H), 7.95 (d, J=9.2 Hz, 2H).

A mixture of Compound 2C (55 mg, 0.2 mmol) and Compound B2 (100 mg, 0.2 mmol) in pyridine (5 mL) was stirred at 70° C. overnight. The reaction mixture was cooled down to room temperature and concentrated. The residue was diluted with water (50 mL) and extracted with ethyl acetate (50 mL×2). The combined organic extracts were washed with water (50 mL×3) and brine (50 mL), dried over anhydrous sodium sulfate, filtered, and concentrated under reduced pressure. The residue was purified by silica gel column chromatography by silica gel column chromatography to give Compound 2D. LC-MS (ESI) m/z: 750 [M+Na]⁺.

Compound 2 was synthesized by employing the procedure described for Compound 1 using Compound 2D in lieu of Compound 1A, which was separated with chiral HPLC to afford Compound 2-1 and Compound 2-2.

Compound 2-1: LC-MS (ESI) m/z: 628 [M+H]⁺; ¹H-NMR (CD₃OD, 400 MHz): δ (ppm) 0.84-0.90 (m, 6H), 1.38-2.034 (m, 13H), 3.50-3.65 (m, 1H), 3.85-3.90 (m, 2H), 4.46-4.69 (m, 3H), 6.79-6.86 (m, 2H), 7.15-7.17 (m, 1H), 7.31-7.50 (m, 5H). Chiral separation condition: MeOH contained 0.5% DEA; OJ-H (4.6×250 mm, 5 μm); retention time: 3.66 minute.

Compound 2-2: LC-MS (ESI) m/z: 628 [M+H]⁺; ¹H-NMR (CD₃OD, 400 MHz): δ (ppm) 0.84-0.90 (m, 6H), 1.36-2.04 (m, 13H), 3.50-3.66 (m, 1H), 3.85-3.90 (m, 2H), 4.46-4.70 (m, 3H), 6.79-6.86 (m, 2H), 7.15-7.17 (m, 1H), 7.31-7.50 (m, 5H). Chiral separation condition: MeOH contained 0.5% DEA; OJ-H (4.6×250 mm, 5 μm); retention time: 2.57 minute.

### Example 3

### Synthesis of N-(3-(3-amino-8-azabicyclo[3.2.1]octan-8-yl)-1-(4-bromophenyl)-1,1-difluoro-3-oxopropan-2-yl)-4′-fluoro-[1,1′-biphenyl]-4-sulfonamide (Compound 3), and N-((2S)-3-(3-amino-8-azabicyclo[3.2.1]octan-8-yl)-1-(4-bromophenyl)-1,1-difluoro-3-oxopropan-2-yl)-4′-fluoro-[1,1′-biphenyl]-4-sulfonamide (Compound 3-2)

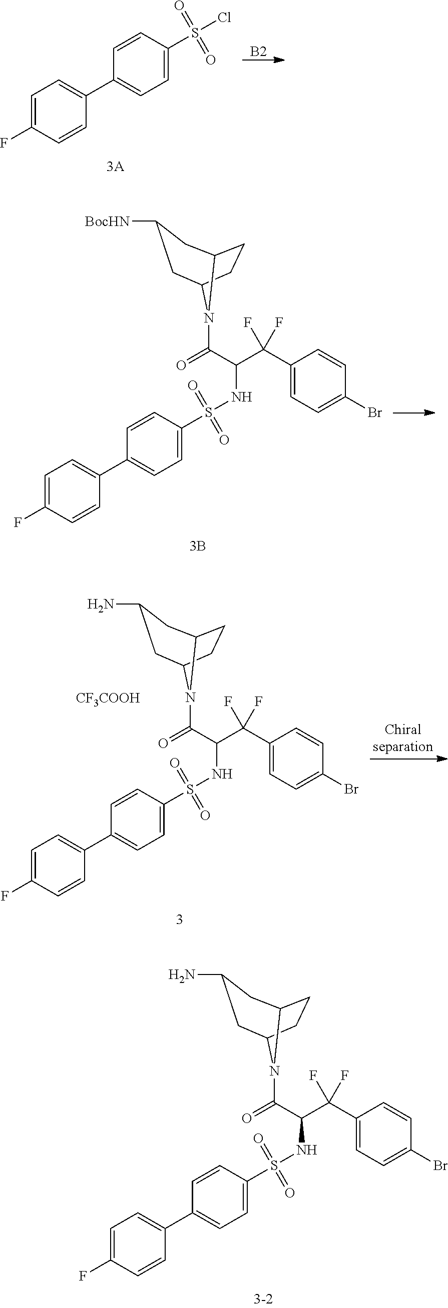

Compounds 3B and 3 were synthesized by employing the procedures described for Compounds 2D and 1 using Compounds 3A and 3B in lieu of Compounds 2C and 1A.

Compound 3B: LC-MS (ESI) m/z: 744 [M+Na]⁺.

Compound 3: LC-MS (ESI) m/z: 622 [M+H]⁺; ¹H-NMR (CDCl₃, 400 MHz): δ (ppm) 1.67-2.05 (m, 8H), 3.55-3.71 (m, 4H), 4.35-4.36 (m, 1H), 4.64-4.69 (m, 2H), 7.06-7.13 (m, 4H), 7.43-7.53 (m, 6H), 8.04-8.09 (m, 2H).

Compound 3 was purified with chiral HPLC to give Compound 3-2. LC-MS (ESI) m/z: 622 [M+H]⁺; ¹H-NMR (CD₃OD, 400 MHz): δ (ppm) 1.10-1.30 (m, 4H), 1.71-1.89 (m, 6H), 3.13-3.16 (m, 1H), 4.34-4.51 (m, 2H), 4.66-4.71 (m, 1H), 4.75-4.79 (m, 1H), 7.21-7.26 (m, 2H), 7.35-7.37 (m, 1H), 7.43-7.45 (m, 1H), 7.51-7.53 (m, 1H), 7.58-7.61 (m, 1H), 7.68-7.80 (m, 6H). Chiral separation condition: MeOH contained 0.5% DEA; OJ-H (4.6×250 mm, 5 μm); retention time: 3.08 minute.

### Example 4

### Synthesis of N-(3-(3-amino-8-azabicyclo[3.2.1]octan-8-yl)-1-(4-bromophenyl)-1,1-difluoro-3-oxopropan-2-yl)-2-(6-isopropoxynaphthalen-2-yl)ethanesulfonamide trifluoroacetate (Compound 4)

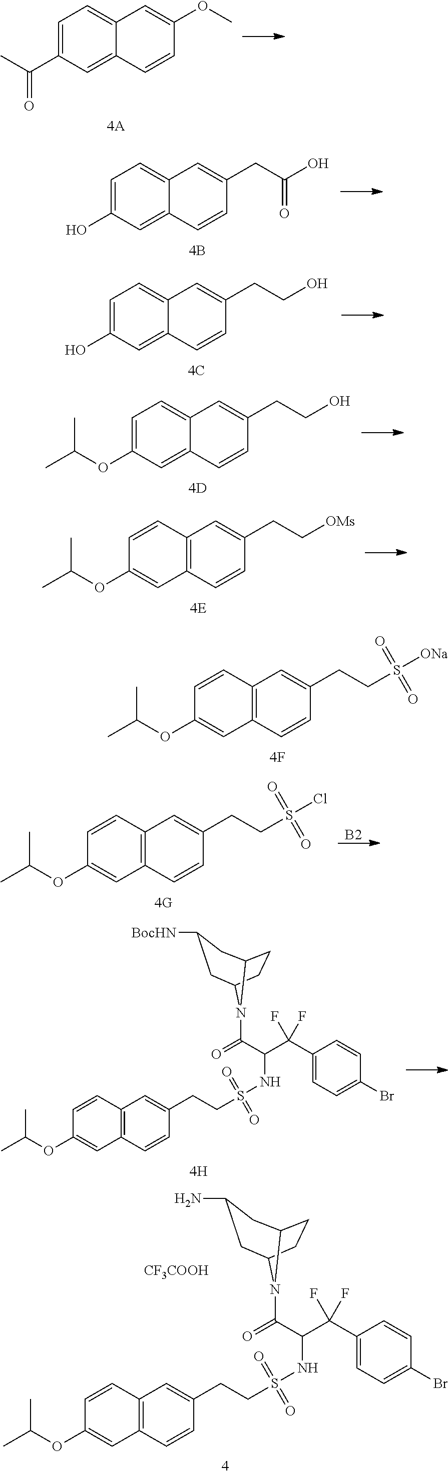

A mixture of Compound 4A (11 g, 55 mmol), sulfur (2.8 g), and morpholine (9.6 g, 0.11 mol) was heated at 140° C. for 18 hours. After removal of the excess morpholine, to the mixture was added concentrated HCl (38 mL) and glacial acetic acid (22 mL) and refluxed for 24 hours. The resulting precipitate was removed by filtration, to the filtrate was added water (100 mL), and the resulting solid was obtained by filtration. The solid was dissolved in a hot solution of Na₂CO₃ (12.0 g) in water (100 mL) and acidified with a diluted HCl solution to form a solid. The solid was filtered and washed with Et₂O to give Compound 4B. LC-MS (ESI) m/z: 201 [M−1]⁺; ¹H NMR (CDCl₃, 400 MHz): δ (ppm) 3.66 (s, 2H), 7.06-7.09 (m, 2H), 7.29 (d, J=9.6 Hz, 1H), 7.61-7.63 (m, 2H), 7.70 (d, J=8.4 Hz, 1H), 9.71 (s, 1H), 12.35 (s, 1H).

To a suspension of LiAlH₄ (470 mg, 12 mmol) in THF (20 mL) was dropped a solution of Compound 4B (2 g, 10 mmol) in THF (5 mL) at 0° C. under N₂ and heated at reflux for 12 hours. To the cooled reaction mixture was carefully added water and its pH was adjusted to about 7 with a diluted HCl solution. It was extracted EtOAc (50 mL×2). The combined organic extracts were washed with brine (100 mL), dried over Na₂SO₄, filtered, and concentrated to provide a residue. The residue was purified by silica gel chromatography (25% ethyl acetate in petroleum) to afford Compound 4C. LC-MS (ESI) m/z: 189 [M+1]⁺.

To a solution of Compound 4C (1.5 g, 8.0 mmol) in DMF (20 mL) was added 2-iodopropane (1.2 mL), Cs₂CO₃ (6.5 g, 20 mmol). The reaction mixture was stirred at room temperature overnight and filtered. The filtrate was diluted with dichloromethane and washed with water (50 mL×2) and brine (50 mL). The combined organic layer was dried over Na₂SO₄, filtered, and concentrated to provide a residue. The residue was purified by silica gel column chromatography to get Compound 4D. LC-MS (ESI) m/z: 231 [M+1]⁺. ¹H NMR (CDCl₃, 400 MHz): δ (ppm) 1.38-1.40 (m, 6H), 2.99 (t, J=6.8 Hz, 2H), 3.89-3.94 (m, 2H), 4.67-4.71 (m, 1H), 7.09-7.12 (m, 2H), 7.30 (d, J=10 Hz, 1H), 7.59 (s, 1H), 7.65-7.69 (m, 2H).

To a solution of Compound 4D (1.7 g, 7.4 mmol) in dichloromethane (50 mL) was added Et₃N (2.24 g, 2.2 mmol), MsCl (1.27 g, 11 mmol) dropwised at 0° C. The reaction mixture was stirred at room temperature for 2 hours, diluted with dichloromethane (20 mL), added water (50 mL), and extracted with dichloromethane (50 mL×2). The combined organic extracts were washed with brine (50 mL), dried over Na₂SO₄, filtered, and concentrated under reduced pressure to provide a residue. The residue was purified by silica gel column chromatography to get Compound 4E. LC-MS (ESI) m/z: 309 [M+1]⁺. ¹H NMR (CDCl₃, 400 MHz): δ (ppm) 1.39-1.41 (m, 6H), 2.82 (s, 3H), 3.17 (t, J=6.8 Hz, 2H), 4.48 (t, J=6.4 Hz, 2H), 4.67-4.71 (m, 1H), 7.12 (s, 2H), 7.28-7.31 (m, 1H), 7.59-7.69 (m, 3H).

To a solution of Compound 4E (1.7 g, 5.5 mmol) in dioxane (10 mL) was added Na₂SO₃ (2.1 g, 16.5 mmol) and water (10 mL). It was heated at 90° C. overnight, cooled to room temperature, added dichloromethane (10 mL), and filtered to give Compound 4F. LC-MS (ESI) m/z: 295 [M−Na+2H]⁺.

A mixture of Compound 4F (300 mg, 0.95 mmol) in POCl₃ (5 mL) was heated at 100° C. for 3 hours and centrated to remove POCl₃. To the reaction mixture was added ice water, diluted with dichloromethane (50 mL), added a diluted NaHCO₃ to adjust its pH to about 7, and extracted with dichloromethane (20 mL×2). The combined organic extracts were washed with brine (50 mL), dried over Na₂SO₄, and concentrated under reduced pressure to give Compound 4G: LC-MS (ESI) m/z: 313 [M+1]⁺.

Compounds 4H and 4 were synthesized by employing the procedures described for Compounds 2D and 1 using Compounds 4G and 4H in lieu of Compounds 2C and 1A.

Compound 4H: LC-MS (ESI) m/z: 764 [M+H]⁺.

Compound 4: LC-MS (ESI) m/z: 664 [M+H]⁺; ¹H-NMR (CDCl₃, 400 MHz): δ (ppm) 1.28 (s, 3H), 1.29 (s, 3H), 1.56-1.96 (m, 7H), 2.81-2.88 (m, 3H), 3.06 (s, 2H), 3.27-3.50 (m, 2H), 4.11 (s, 1H), 4.54-4.57 (m, 2H), 4.71 (s, 1H), 4.92 (s, 2H), 6.98-7.00 (m, 3H), 7.21-7.28 (m, 3H), 7.37-7.38 (m, 2H), 7.51 (t, J=10.0 Hz, 2H), 7.89 (s, 1H).

### Example 5

### Synthesis of N-(3-(3-amino-8-azabicyclo[3.2.1]octan-8-yl)-1-(4-bromophenyl)-1,1-difluoro-3-oxopropan-2-yl)-6-(cyclopentyloxy)naphthalene-2-sulfonamide trifluoroacetate (Compound 5), N-((2R)-3-(3-amino-8-azabicyclo[3.2.1]octan-8-yl)-1-(4-bromophenyl)-1,1-difluoro-3-oxopropan-2-yl)-6-(cyclopentyloxy)naphthalene-2-sulfonamide (Compound 5-1), and N-((2S)-3-(3-amino-8-azabicyclo[3.2.1]octan-8-yl)-1-(4-bromophenyl)-1,1-difluoro-3-oxopropan-2-yl)-6-(cyclopentyloxy)naphthalene-2-sulfonamide trifluoroacetate (Compound 5-2)

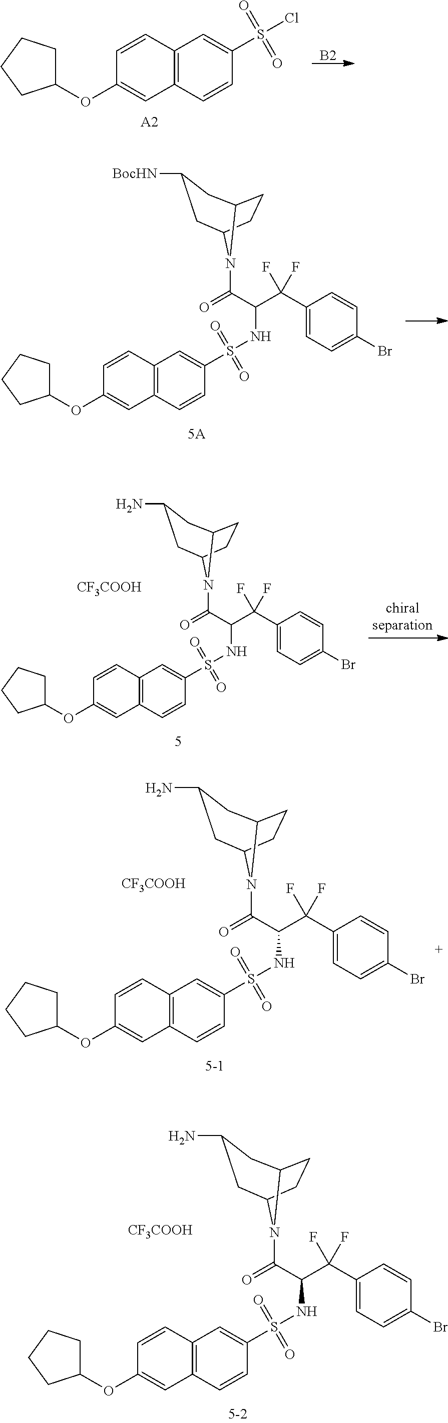

Compounds 5A and 5 were synthesized by employing the procedures described for Compounds 1A and 1 using Compound A2 and Compound 5A in lieu of Compound A1 and Compound 1A.

Compound 5A: LC-MS (ESI) m/z: 762 [M+H]⁺.

Compound 5: LC-MS (ESI) m/z: 662 [M+H]⁺; ¹H-NMR (CD₃OD, 400 MHz): δ (ppm) 1.28-2.18 (m, 16H), 3.39-3.76 (m, 1H), 4.56-4.55 (m, 1H), 4.65 (t, 1H), 4.84-5.06 (m, 2H), 7.17-7.303 (m, 4H), 7.38-7.57 (m, 3H), 7.69-7.86 (m, 2H), 8.09 (d, 1H), which was separated by chiral HPLC (MeOH contained 0.1% DEA; IC 250×4.6 mm, 5 m) to give two isomers Compound 5-1 and Compound 5-2.

Compound 5-1: LC-MS (ESI) m/z: 662 [M+H]⁺; ¹H-NMR (CD₃OD, 400 MHz): δ (ppm) 0.77-2.20 (m, 16H), 3.49-3.80 (m, 1H), 4.40-4.46 (m, 1H), 4.66-5.10 (m, 3H), 7.19-7.31 (m, 4H), 7.41-7.59 (m, 3H), 7.71-7.88 (m, 2H), 8.13 (d, J=38.8 Hz, 1H). Chiral separation condition: MeOH contained 0.1% DEA; IC (250×4.6 mm, 5 μm); retention time: 4.48 minute.

Compound 5-2: LC-MS (ESI) m/z: 662 [M+H]⁺; ¹H-NMR (CD₃OD, 400 MHz): δ (ppm) 0.77-2.20 (m, 16H), 3.49-3.78 (m, 1H), 4.40-4.46 (m, 1H), 4.66-5.05 (m, 3H), 7.17-7.31 (m, 4H), 7.41-7.59 (m, 3H), 7.71-7.88 (m, 2H), 8.13 (d, J=38.8 Hz, 1H). Chiral separation condition: MeOH contained 0.1% DEA; IC (250×4.6 mm, 5 μm); retention time: 3.37 minute.

### Example 6

### Synthesis of N-((2R)-3-(3-amino-8-azabicyclo[3.2.1]octan-8-yl)-1-(4-bromophenyl)-1,1-difluoro-3-oxopropan-2-yl)-6-butoxynaphthalene-2-sulfonamide trifluoroacetate (Compound 6-1), and N-((2S)-3-(3-amino-8-azabicyclo[3.2.1]octan-8-yl)-1-(4-bromophenyl)-1,1-difluoro-3-oxopropan-2-yl)-6-butoxynaphthalene-2-sulfonamide trifluoroacetate (Compound 6-2)

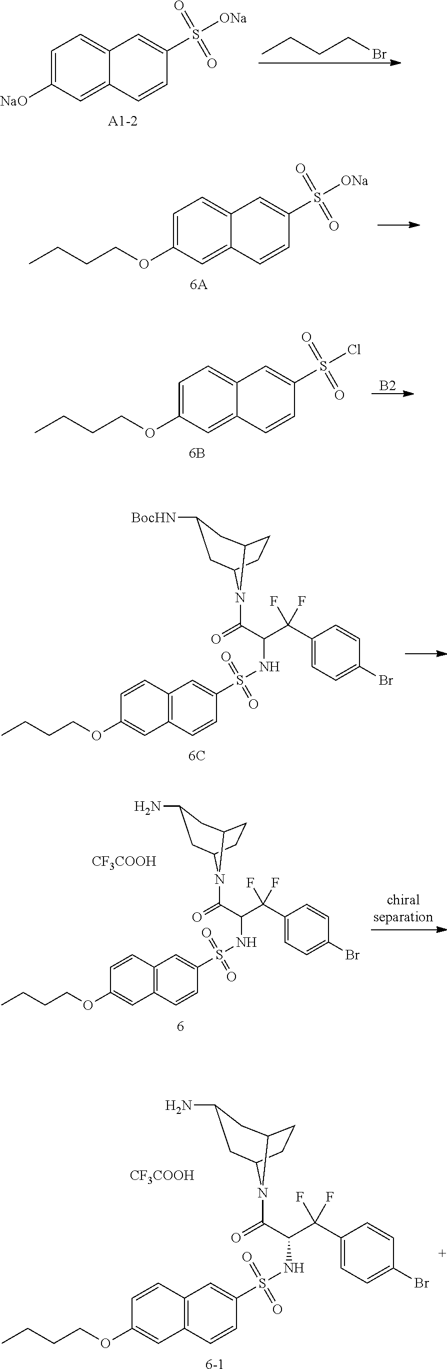

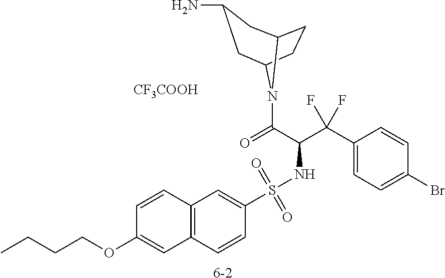

Compounds 6A, 6B, and 6C were synthesized by employing the procedures described for Compounds A1-3, A1, and Compound 1A using bromobutane, Compounds 6A, and 6B in lieu of 2-bromopropane, Compounds A1-3, and A1.

Compound 6A: LC-MS (ESI) m/z: 315 [M+H]⁺.

Compound 6B: LC-MS (ESI) m/z: non-ionizable compound under routine conditions used.

Compound 6C: LC-MS (ESI) m/z: 772 [M+Na]⁺; ¹H-NMR (CDCl₃, 400 MHz): δ (ppm) 0.39-0.72 (m, 1H), 0.99-1.03 (m, 3H), 1.25-1.37 (m, 2H), 1.41-1.46 (m, 10H), 1.52-1.57 (m, 4H), 1.80-1.89 (m, 5H), 3.74-3.88 (m, 1H), 4.09-4.23 (m, 3H), 4.39-4.53 (m, 2H), 5.93-6.07 (m, 1H), 7.13-7.31 (m, 4H), 7.46-7.51 (m, 2H), 7.56-7.66 (m, 1H), 7.71-7.82 (m, 2H), 8.16-8.22 (m, 1H).

To a solution of Compound 6C (78 mg, 0.104 mmol) in dichloromethane (5 mL) was added 2,2,2-trifluoroacetic acid (0.5 mL) and stirred at room temperature for 2 hours. The reaction mixture was diluted with dichloromethane (100 mL), washed with water (50 mL×2) and brine (50 mL), dried over anhydrous sodium sulfate, filtered, and concentrated to provide a crude product. The crude product was purified with preparative HPLC to give Compound 6, which was separated with chiral HPLC to give Compound 6-1 and Compound 6-2.

Compound 6-1: LC-MS (ESI) m/z: 650 [M+H]⁺; ¹H-NMR (CD₃OD, 400 MHz): δ (ppm) 1.01-1.06 (m, 3H), 1.35-1.44 (m, 1H), 1.52-1.64 (m, 3H), 1.72-2.21 (m, 8H), 3.49-3.77 (m, 1H), 4.13-4.20 (m, 2H), 4.34-4.57 (m, 1H), 4.65-4.72 (m, 2H), 7.18-7.43 (m, 4H), 7.45-7.60 (m, 3H), 7.73-7.89 (m, 2H), 8.09-8.19 (m, 1H). Chiral separation condition: MeOH contained 0.5% DEA; IC (4.6×250 mm, 5 μm); retention time: 6.26 minute.

Compound 6-2: LC-MS (ESI) m/z: 650 [M+H]⁺; ¹H-NMR (CD₃OD, 400 MHz): δ (ppm) 1.01-1.06 (m, 3H), 1.35-1.44 (m, 1H), 1.52-1.64 (m, 3H), 1.72-2.21 (m, 8H), 3.49-3.77 (m, 1H), 4.13-4.20 (m, 2H), 4.34-4.57 (m, 1H), 4.65-4.72 (m, 2H), 7.18-7.43 (m, 4H), 7.45-7.60 (m, 3H), 7.73-7.89 (m, 2H), 8.09-8.19 (m, 1H). Chiral separation condition: MeOH contained 0.5% DEA; IC (4.6×250 mm, 5 μm); retention time: 4.91 minute.

### Example 7

### Synthesis of N-((2R)-3-(3-amino-8-azabicyclo[3.2.1]octan-8-yl)-1-(4-bromophenyl)-1,1-difluoro-3-oxopropan-2-yl)-6-isobutoxynaphthalene-2-sulfonamide (Compound 7-1), and N-((2S)-3-(3-amino-8-azabicyclo[3.2.1]octan-8-yl)-1-(4-bromophenyl)-1,1-difluoro-3-oxopropan-2-yl)-6-isobutoxynaphthalene-2-sulfonamide (Compound 7-2)

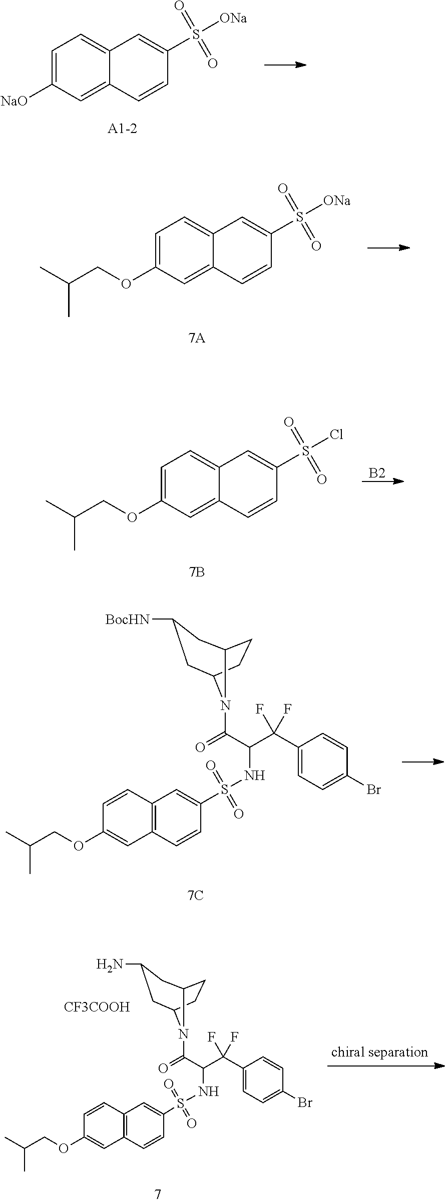

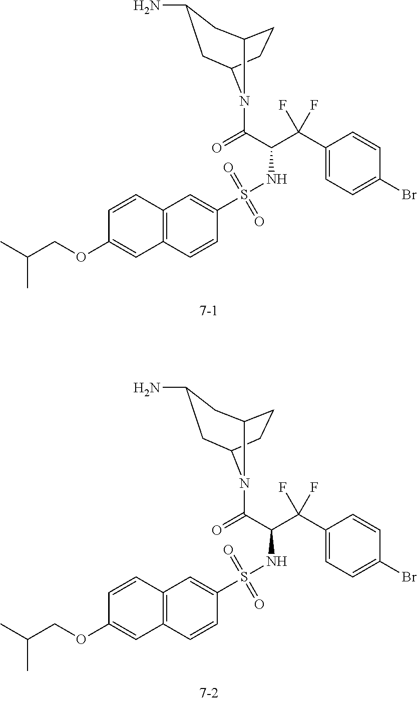 [SMILES: CC(C)COc1ccc2cc(S(=O)(=O)N[C@H](C(=O)N3C4CCC3CC(N)C4)C(F)(F)c3ccc(Br)cc3)ccc2c1 ; CC(C)COc1ccc2cc(S(=O)(=O)N[C@@H](C(=O)N3C4CCC3CC(N)C4)C(F)(F)c3ccc(Br)cc3)ccc2c1]

Compounds 7A, 7B, 7C, and 7 were synthesized by employing the procedures described for Compounds A1-3, A1, Compounds 2D, and 1 using 1-bromo-2-methylpropane, Compounds 7A, 7B, and 7C in lieu of 2-bromopropane, Compounds A1-3, Compounds 2C, and 1A.

Compound 7A: LC-MS (ESI) m/z: 298 [M+H₂O—Na]⁺.

Compound 7B: ¹H-NMR (CDCl₃, 400 MHz): δ (ppm) 1.09 (d, J=6.4 Hz, 6H), 2.18-2.19 (m, 1H), 3.89 (d, J=3.2 Hz, 2H), 7.19 (s, 1H), 7.31-7.34 (m, 1H), 7.87-7.95 (m, 3H), 8.49 (s, 1H).

Compound 7C: LC-MS (ESI) m/z: 772 [M+Na]⁺.

Compound 7, which was separated with chiral HPLC (MeOH contained 0.1% DEA; IC, 250×4.6 mm, 5 m) to give Compound 7-1 and Compound 7-2.

Compound 7-1: LC-MS (ESI) m/z: 650 [M+H]⁺; ¹H-NMR (CD₃OD, 400 MHz): δ (ppm) 0.31-0.92 (m, 2H), 0.98 (d, J=6.4 Hz, 6H), 1.09-1.22 (m, 2H), 1.44-1.60 (m, 4H), 2.01-2.07 (m, 1H), 2.71-2.98 (m, 1H), 3.80 (d, J=6.4 Hz, 2H), 4.08-4.28 (m, 2H), 4.52-4.61 (m, 1H), 7.15-7.33 (m, 4H), 7.39-7.61 (m, 3H), 7.68-7.82 (m, 2H), 8.09-8.16 (m, 1H). Chiral separation condition: MeOH contained 0.1% DEA; IC (4.6×250 mm, 5 μm); retention time: 5.00 minute.

Compound 7-2: LC-MS (ESI) m/z: 650 [M+H]⁺; ¹H-NMR (CD₃OD, 400 MHz): δ (ppm) 0.35-0.72 (m, 2H), 0.98 (d, J=6.4 Hz, 6H), 1.12-1.19 (m, 2H), 1.45-1.57 (m, 4H), 2.01-2.05 (m, 1H), 2.71-2.98 (m, 1H), 3.80 (d, J=6.4 Hz, 2H), 4.09-4.29 (m, 2H), 4.56-4.61 (m, 1H), 7.16-7.33 (m, 4H), 7.38-7.60 (m, 3H), 7.68-7.81 (m, 2H), 8.09-8.16 (m, 1H). Chiral separation condition: MeOH contained 0.1% DEA; IC (4.6×250 mm, 5 μm); retention time: 4.62 minute.

### Example 8

### Synthesis of N-((2R)-3-(3-amino-8-azabicyclo[3.2.1]octan-8-yl)-1-(4-chlorophenyl)-1,1-difluoro-3-oxopropan-2-yl)-6-isopropoxynaphthalene-2-sulfonamide trifluoroacetate (Compound 8-1), and N-((2S)-3-(3-amino-8-azabicyclo[3.2.1]octan-8-yl)-1-(4-chlorophenyl)-1,1-difluoro-3-oxopropan-2-yl)-6-isopropoxynaphthalene-2-sulfonamide trifluoroacetate (Compound 8-2)

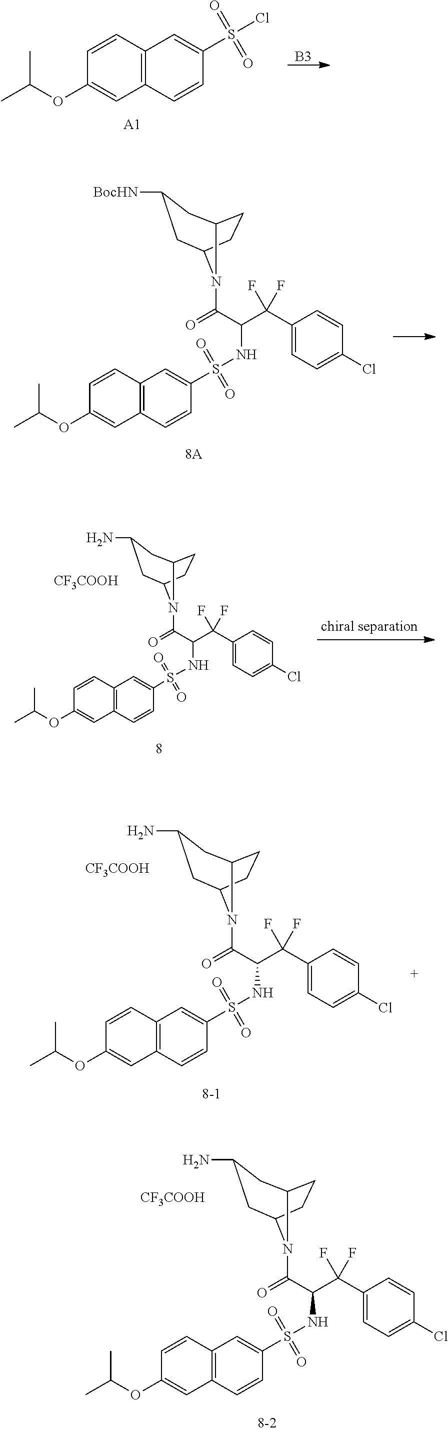

Compounds 8A and 8 were synthesized by employing the procedures described for Compounds 2D and 1 using Compounds A1, B3, and Compound 8A in lieu of Compound 2C, Compound B2, and Compound 1A.

Compound 8A, which was used in the next step without further purification. LC-MS (ESI) m/z: 714 [M+Na]⁺.

Compound 8, which was separated with chiral HPLC (MeOH contained 0.1% DEA; IC, 150×4.6 mm, 5 m) to give Compound 8-1 and Compound 8-2.

Compound 8-1: LC-MS (ESI) m/z: 592 [M+H]⁺; ¹H-NMR (CD₃OD, 400 MHz): δ (ppm) 0.72-1.31 (m, 1H), 1.37-1.39 (m, 6H), 1.43-1.50 (m, 1H), 1.71-2.21 (m, 5H), 3.47-3.77 (m, 1H), 4.34-4.55 (m, 1H), 4.65-4.87 (m, 4H), 7.01 (d, J=8.4 Hz, 1H), 7.24 (d, J=16.0 Hz, 2H), 7.34 (t, J=8.4 Hz, 2H), 7.44-7.59 (m, 2H), 7.69-7.87 (m, 2H), 8.06-8.17 (m, 1H). Chiral separation condition: MeOH contained 0.1% DEA; IC (150×4.6 mm, 5 μm); retention time: 5.13 minute.

Compound 8-2: LC-MS (ESI) m/z: 592 [M+H]⁺; ¹H-NMR (CD₃OD, 400 MHz): δ (ppm) 0.72-1.33 (m, 1H), 1.37-1.39 (m, 6H), 1.43-1.51 (m, 1H), 1.62-2.20 (m, 5H), 3.47-3.77 (m, 1H), 4.33-4.55 (m, 1H), 4.65-4.88 (m, 4H), 7.01 (d, J=8.4 Hz, 1H), 7.24 (d, J=16.0 Hz, 2H), 7.34 (t, J=8.4 Hz, 2H), 7.44-7.58 (m, 2H), 7.69-7.87 (m, 2H), 8.07-8.17 (m, 1H). Chiral separation condition: MeOH contained 0.1% DEA; IC (150×4.6 mm, 5 μm); retention time: 3.94 minute.

### Example 9

### Synthesis of N-((2R)-3-(3-amino-8-azabicyclo[3.2.1]octan-8-yl)-1-(4-bromophenyl)-1,1-difluoro-3-oxopropan-2-yl)-2-(6-isopropoxynaphthalen-2-yl)-2-oxoacetamide trifluoroacetate (Compound 9-1), and N-((2S)-3-(3-amino-8-azabicyclo[3.2.1]octan-8-yl)-1-(4-bromophenyl)-1,1-difluoro-3-oxopropan-2-yl)-2-(6-isopropoxynaphthalen-2-yl)-2-oxoacetamide trifluoroacetate (Compound 9-2)

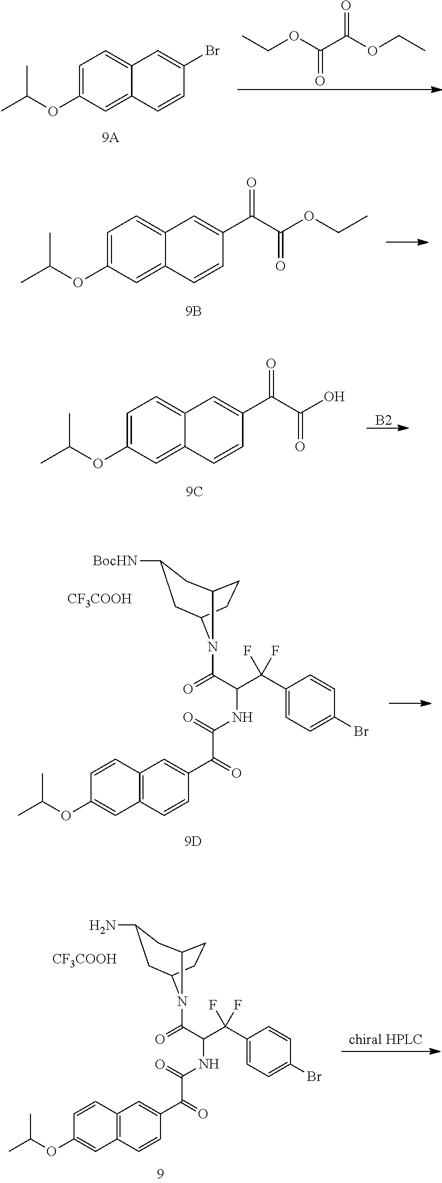

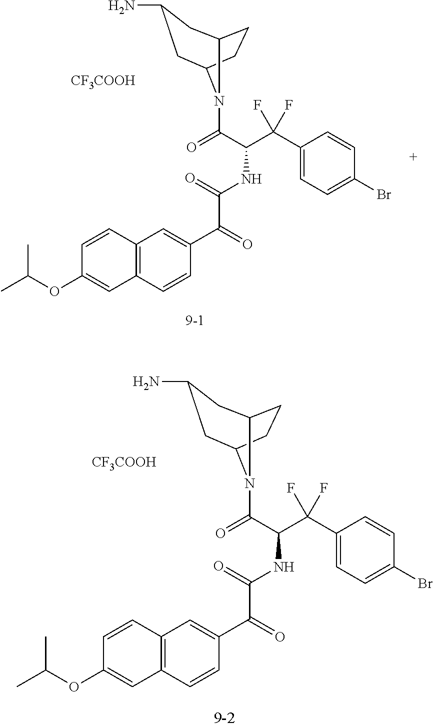 [SMILES: CC(C)Oc1ccc2cc(C(=O)C(=O)N[C@H](C(=O)N3C4CCC3CC(N)C4)C(F)(F)c3ccc(Br)cc3)ccc2c1 ; "CF3COOH" ; CC(C)Oc1ccc2cc(C(=O)C(=O)N[C@@H](C(=O)N3C4CCC3CC(N)C4)C(F)(F)c3ccc(Br)cc3)ccc2c1 ; "CF3COOH"]

To a solution of Compound 9A (17.3 g, 65 mmol) in THF (300 mL) was dropped a solution of n-BuLi in hexane (2.5 M, 26 mL, 65 mmol) under nitrogen atmosphere at −60° C. and stirred for 30 minutes. The solution was added to a solution of diethyl oxalate (28.5 g, 195 mmol) in THF (200 mL) at −60° C., stirred at −60° C. for 2 hours, quenched with saturated ammonium chloride solution, and extracted with ethyl acetate (400 mL×2). The combined organic extracts were washed with brine (50 mL), dried over anhydrous sodium sulfate, filtered, and concentrated to provide a residue. The residue was purified by silica gel column chromatography to yield Compound 9B. LC-MS (ESI) m/z: 287 [M+H]⁺.

To a solution of Compound 9B (400 mg, 1.40 mmol) in THF (5 mL) was added LiOH—H₂O (88 mg, 2.10 mmol) and H₂O (2.0 mL). The mixture was stirred at 15° C. overnight. The reaction mixture was neutralized with 1 NHCl solution and concentrated under reduced pressure to provide a residue. The residue was dissolved in H₂O (5 mL) and extracted with ethyl acetate (10 mL×2). The combined organic extracts were dried over anhydrous sodium sulfate, filtered, and concentrated to give Compound 9C. LC-MS (ESI) m/z: 259.1 [M+H]⁺.

Compounds 9D and 9 were synthesized by employing the procedures described for Compound B4 and Compound 1 using Compound B2, Compounds 9C without using DIPEA as base, and 9D in lieu of Compounds B4-1, B2-6 using DIPEA as base, and Compound 1A.

Compound 9D: LC-MS (ESI) m/z: 628.1 [M−99]⁺.

Compound 9, which was separated with chiral HPLC (n-hexane/EtOH contained 0.1% DEA, 70/30; OZ-H, 4.6×250 mm, 5 m) to give Compound 9-1 and Compound 9-2.

Compound 9-1: LC-MS (ESI) m/z: 628.3 [M+H]⁺; ¹H-NMR (acetone-d₆, 400 MHz): δ (ppm) 1.41 (d, J=6.4 Hz, 6H), 1.87-2.05 (m, 5H), 2.07-2.24 (m, 4H), 4.64-4.92 (m, 4H), 5.68-5.72 (m, 1H), 7.22-7.27 (m, 1H), 7.41 (s, 1H), 7.62-7.74 (m, 4H), 7.85-7.99 (m, 3H), 8.50-8.65 (m, 1H). Chiral separation condition: n-hexane/EtOH contained 0.1% DEA, 70/30; IA (4.6×250 mm, 5 μm); retention time: 11.91 minute.

Compound 9-2: LC-MS (ESI) m/z: 628.3 [M+H]⁺; ¹H-NMR (acetone-d₆, 400 MHz): δ (ppm) 1.34 (d, J=7.2 Hz, 6H), 1.40-2.05 (m, 5H), 2.07-2.44 (m, 5H), 3.00-3.23 (m, 1H), 4.67-4.92 (m, 4H), 5.68-5.72 (m, 1H), 7.22-7.25 (m, 1H), 7.41 (s, 1H), 7.62-7.74 (m, 4H), 7.85-8.02 (m, 3H), 8.50-8.65 (m, 1H). Chiral separation condition: n-hexane/EtOH contained 0.1% DEA, 70/30; IA (4.6×250 mm, 5 μm); retention time: 9.25 minute.

### Example 10

### Synthesis of N-((2R)-3-(3-amino-8-azabicyclo[3.2.1]octan-8-yl)-1-(4-bromophenyl)-1,1-difluoro-3-oxopropan-2-yl)-6-(pyridin-2-yloxy)naphthalene-2-sulfonamide trifluoroacetate (Compound 10-1)

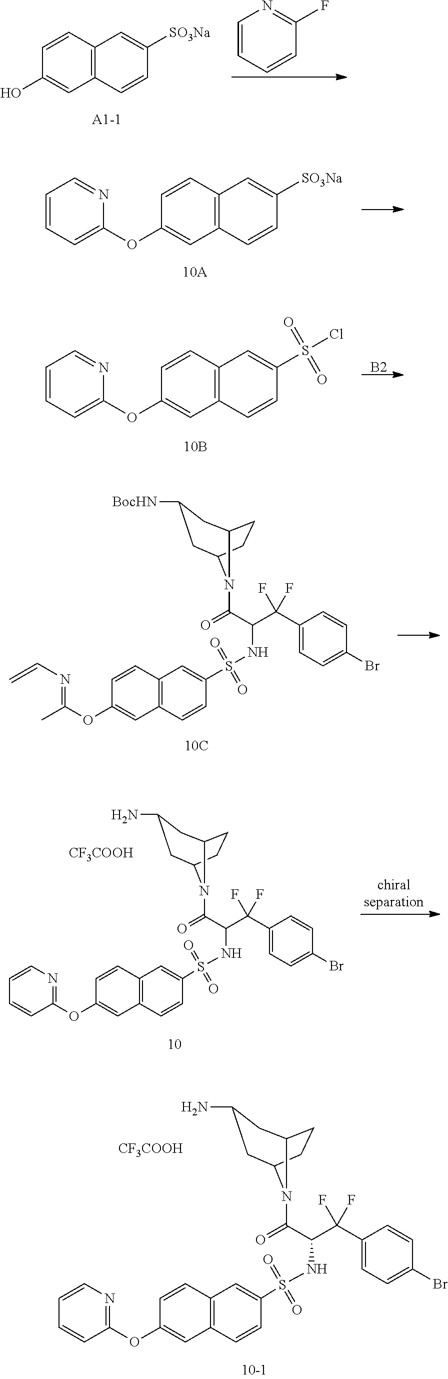

A mixture of Compound A1-1 (2 g, 8.1 mmol), 2-fluoropyridine (1.6 g, 16.2 mmol), and t-BuOK (2.3 g, 20 mmol) in DMSO (20 mL) was heated at 120° C. overnight. The reaction mixture was cooled to room temperature, treated with acetone (50 mL), and filtered. The filtrate was concentrated and purified with reverse phase chromatography using eluent (methanol in water, from 0% to 50% v/v) to furnish Compound 10A. LC-MS (ESI) m/z: 300 [M−Na]⁺; ¹H NMR (CDCl₃, 400 MHz): δ (ppm) 6.17 (d, J=8.0 Hz, 1H), 6.88 (t, J=8.4 Hz, 1H), 6.96 (d, J=8.4 Hz, 1H), 7.36-7.42 (m, 1H), 7.64 (t, J=4.4 Hz, 1H), 7.78 (d, J=8.0 Hz, 1H), 8.36 (d, J=18.8 Hz, 2H), 9.11 (s, 1H), 9.54 (s, 1H).

Compounds 10B, 10C, and 10 were synthesized by employing the procedures described for Compound A1, Compounds 2D, and 1 using Compounds 10A, 10B, and 10C in lieu of Compound A1-3, Compounds 2C, and 1A.

Compound 10B. LC-MS (ESI) m/z: 320 [M+H]⁺.

Compound 10C. LC-MS (ESI) m/z: 771 [M+H]⁺.

Compound 10 was separated with chiral HPLC to give two enantiomers. Compound 10-1: LC-MS (ESI) m/z: 671 [M+H]⁺; ¹H-NMR (CD₃OD, 400 MHz): δ (ppm) 0.87-2.19 (m, 8H), 3.54-3.75 (m, 1H), 4.38-4.77 (m, 3H), 7.09-7.14 (m, 1H), 7.19-7.26 (m, 3H), 7.43-7.48 (m, 2H), 7.50-7.52 (m, 1H), 7.55-7.66 (m, 2H), 7.81-7.87 (m, 1H), 7.90-7.94 (m, 1H), 8.05 (t, J=8.0 Hz, 1H), 8.20 (d, J=5.2 Hz, 1H), 8.28 (d, J=23.6 Hz, 1H). Chiral separation condition: n-hexane/EtOH contained 0.1% DEA, 40/60; AD-H (250×4.6 mm, 5 μm); retention time: 9.68 minute.

### Example 11

### Synthesis of N-((2R)-3-(3-amino-8-azabicyclo[3.2.1]octan-8-yl)-1-(4-bromophenyl)-1,1-difluoro-3-oxopropan-2-yl)-4-(benzyloxy)benzenesulfonamide trifluoroacetate (Compound 11-1), and N-((2S)-3-(3-amino-8-azabicyclo[3.2.1]octan-8-yl)-1-(4-bromophenyl)-1,1-difluoro-3-oxopropan-2-yl)-4-(benzyloxy)benzenesulfonamide trifluoroacetate (Compound 11-2)

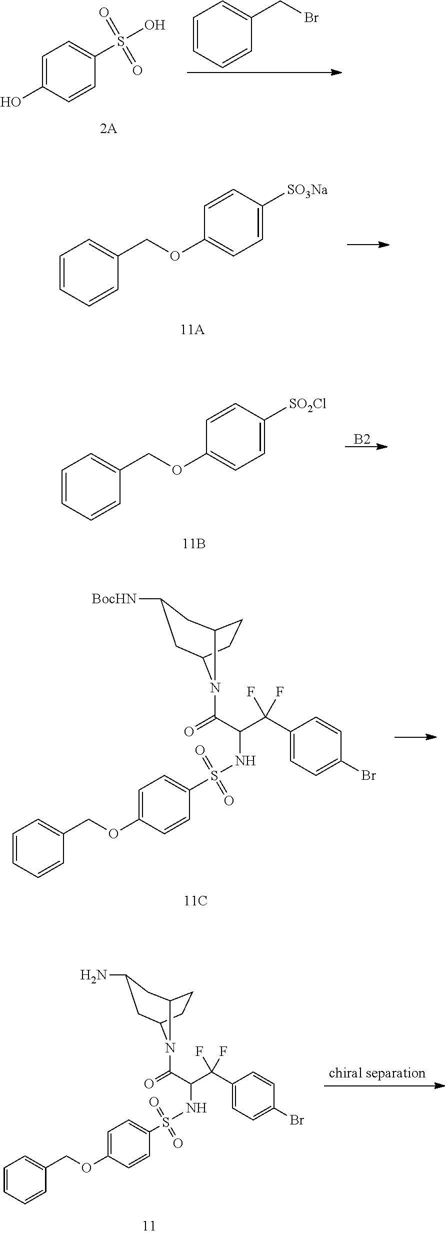

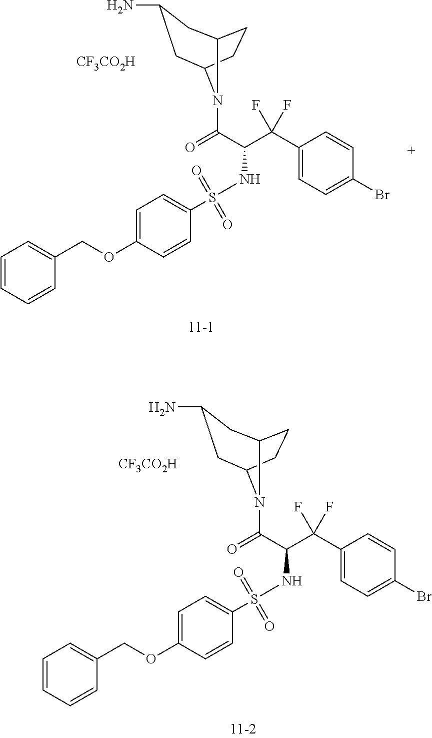 [SMILES: NC1CC2CCC(C1)N2C(=O)[C@@H](NS(=O)(=O)c1ccc(OCc2ccccc2)cc1)C(F)(F)c1ccc(Br)cc1 ; "CF3CO2H" ; NC1CC2CCC(C1)N2C(=O)[C@H](NS(=O)(=O)c1ccc(OCc2ccccc2)cc1)C(F)(F)c1ccc(Br)cc1 ; "CF3CO2H"]

Compounds 11A, 11B, 11C, and 11 were synthesized by employing the procedures described for Compounds A3-1, A1, Compounds 2D, and 1 using (bromomethyl)benzene, Compounds 11A, 11B, and 11C in lieu of (bromomethyl)cyclohexane, Compound A1-3, Compounds 2C, and 1A.

Compound 11A: LC-MS (ESI) m/z: 263 [M−Na]⁺.

Compound 11B: LC-MS (ESI) m/z: No; ¹H-NMR (CDCl₃, 400 MHz): δ (ppm) 5.10 (s, 2H), 7.10 (d, J=9.2 Hz, 2H), 7.35-7.41 (m, 5H), 7.98 (d, J=8.8 Hz, 2H).

Compound 11C: LC-MS (ESI) m/z: 756 [M+Na]⁺.

Compound 11 was separated with chiral HPLC to give Compound 11-1 and Compound 11-2.

Compound 11-1: LC-MS (ESI) m/z: 634 [M+H]⁺; ¹H-NMR (CD₃OD, 400 MHz): δ (ppm) 1.31-1.44 (m, 1H), 1.58-1.65 (m, 1H), 1.73-2.17 (m, 6H), 3.60-3.76 (m, 1H), 4.49-4.85 (m, 3H), 5.18-5.21 (m, 2H), 6.99-7.05 (m, 2H), 7.26-7.28 (m, 1H), 7.33-7.61 (m, 10H). Chiral separation condition: MeOH contained 0.1% DEA; OJ-H (4.6×250 mm, 5 μm); retention time: 4.96 minute.

Compound 11-2: LC-MS (ESI) m/z: 634 [M+H]⁺; ¹H-NMR (CD₃OD, 400 MHz): δ (ppm) 1.35-1.45 (m, 1H), 1.58-1.66 (m, 1H), 1.72-2.17 (m, 6H), 3.60-3.76 (m, 1H), 4.49-4.83 (m, 3H), 5.18-5.21 (m, 2H), 6.99-7.05 (m, 2H), 7.26-7.28 (m, 1H), 7.33-7.62 (m, 10H). Chiral separation condition: MeOH contained 0.1% DEA; OJ-H (4.6×250 mm, 5 μm); retention time: 3.44 minute.

### Example 12

### Synthesis of N-((2R)-3-(3-amino-8-azabicyclo[3.2.1]octan-8-yl)-1-(4-bromophenyl)-1,1-difluoro-3-oxopropan-2-yl)-4-(cyclohexylmethoxy)benzenesulfonamide trifluoroacetate (Compound 12-1)

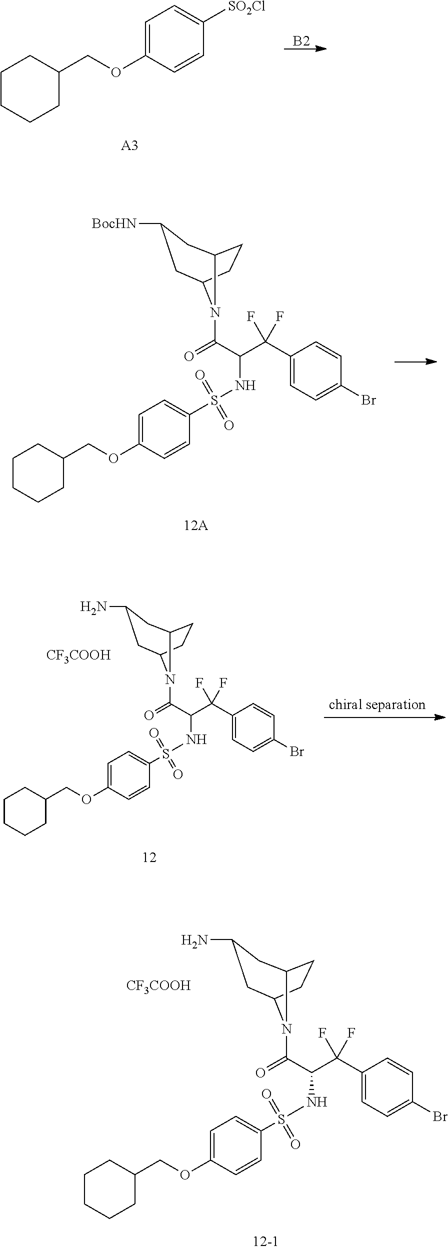

Compounds 12A and 12 were synthesized by employing the procedures described for Compounds 2D and 1 using Compound A3 and Compound 12A in lieu of Compound 2C and Compound 1A.

Compound 12A: LC-MS (ESI) m/z: 762 [M+Na]⁺.

Compound 12 was separated with chiral HPLC to give two enantiomers. Compound 12-1: LC-MS (ESI) m/z: 640 [M+H]⁺; ¹H-NMR (CD₃OD, 400 MHz): δ (ppm) 0.99-1.27 (m, 6H), 1.70-2.04 (m, 13H), 3.54-3.61 (m, 1H), 3.74-3.79 (m, 2H), 4.47-4.71 (m, 3H), 6.77-6.84 (m, 2H), 7.15-7.17 (m, 1H), 7.31-7.34 (m, 2H), 7.38-7.40 (m, 1H), 7.47-7.49 (m, 2H). Chiral separation condition: MeOH contained 0.1% DEA; OJ-H (4.6×250 mm, 5 μm); retention time: 2.02 minute.

### Example 13

### Synthesis of N-((2R)-3-(3-amino-8-azabicyclo[3.2.1]octan-8-yl)-1-(4-bromophenyl)-1,1-difluoro-3-oxopropan-2-yl)-6-((R)-sec-butoxy)naphthalene-2-sulfonamide (Compound 13-1), N-((2S)-3-(3-amino-8-azabicyclo[3.2.1]octan-8-yl)-1-(4-bromophenyl)-1,1-difluoro-3-oxopropan-2-yl)-6-((S)-sec-butoxy)naphthalene-2-sulfonamide (Compound 13-2), N-((2R)-3-(3-amino-8-azabicyclo[3.2.1]octan-8-yl)-1-(4-bromophenyl)-1,1-difluoro-3-oxopropan-2-yl)-6-((S)-sec-butoxy)naphthalene-2-sulfonamide (Compound 13-3), and N-((2S)-3-(3-amino-8-azabicyclo[3.2.1]octan-8-yl)-1-(4-bromophenyl)-1,1-difluoro-3-oxopropan-2-yl)-6-((R)-sec-butoxy)naphthalene-2-sulfonamide (Compound 13-4)

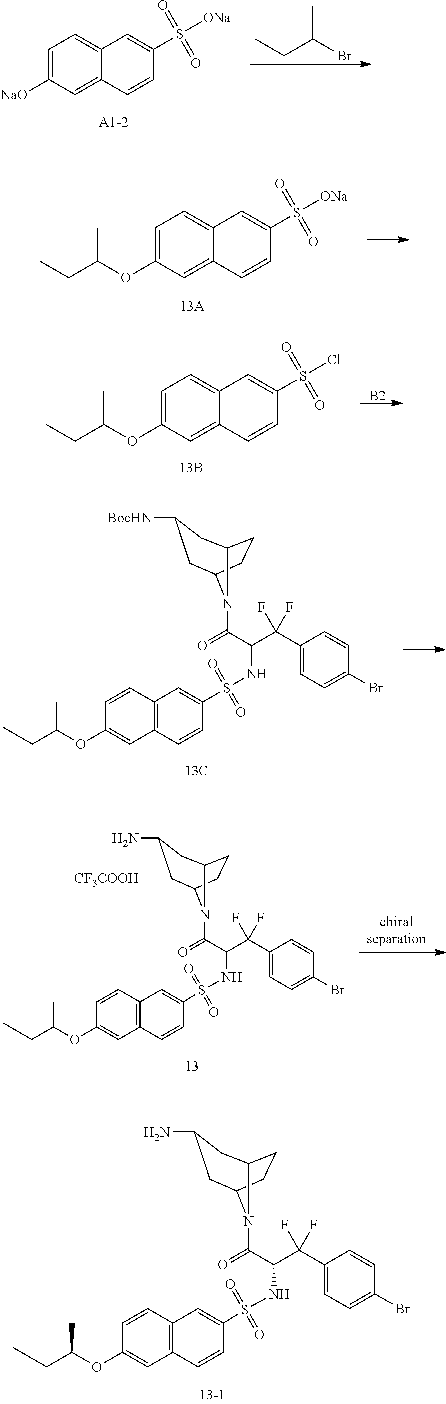

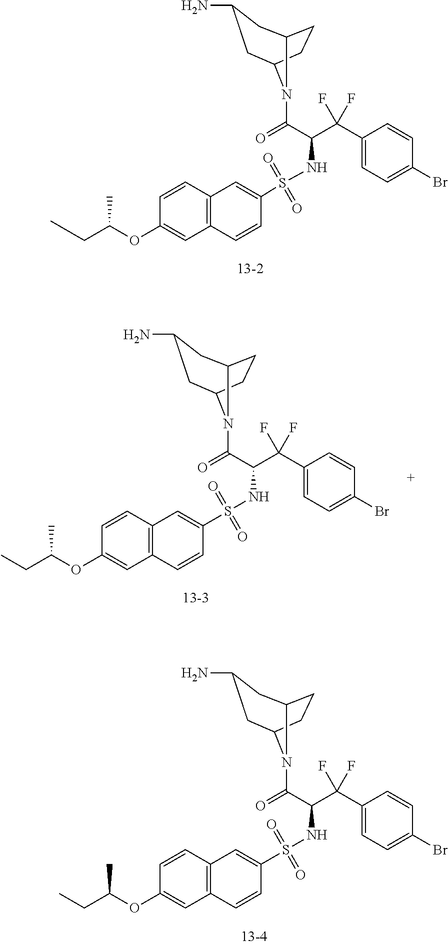 [SMILES: CC[C@H](C)Oc1ccc2cc(S(=O)(=O)N[C@@H](C(=O)N3C4CCC3CC(N)C4)C(F)(F)c3ccc(Br)cc3)ccc2c1 ; CC[C@H](C)Oc1ccc2cc(S(=O)(=O)N[C@H](C(=O)N3C4CCC3CC(N)C4)C(F)(F)c3ccc(Br)cc3)ccc2c1 ; CC[C@@H](C)Oc1ccc2cc(S(=O)(=O)N[C@@H](C(=O)N3C4CCC3CC(N)C4)C(F)(F)c3ccc(Br)cc3)ccc2c1]

Compounds 13A, 13B, 13C, and 13 were synthesized by employing the procedures described for Compounds A1-3, A1, Compounds 2D, and 1 using 1-bromo-2-methylpropane, Compounds 13A, 13B, and 13C in lieu of 2-bromopropane, Compound A1-3, Compounds 2C, and 1A.

Compound 13A: LC-MS (ESI) m/z: 298 [M−Na+H+H₂O]⁺.

Compound 13B: LC-MS (ESI) m/z: non-ionizable compound under routine conditions used; ¹H-NMR (CDCl₃, 400 MHz): δ (ppm) 0.96 (t, J=7.2 Hz, 3H), 1.28 (d, J=6 Hz, 3H), 1.62-1.78 (m, 2H), 4.53-4.57 (m, 1H), 7.14 (dd, J=2.4, 8.8 Hz, 1H), 7.30 (s, 1H), 7.64 (dd, J=2.4, 8.8 Hz, 1H), 7.72 (d, J=8.4 Hz, 1H), 7.86 (d, J=8.4 Hz, 1H), 8.04 (s, 1H).

Compound 13C, which was used directly for the next step without further purification. LC-MS (ESI) m/z: 772 [M+Na]⁺.

Compound 13 was purified with preparative HPLC to give two mixtures of two diastereoisomers. The mixtures were further separated with chiral HPLC (MeOH contained 0.1% DEA; IC 250×4.6 mm, 5 m) to afford Compound 13-1, Compound 13-2, Compound 13-3, and Compound 13-4.

Compound 13-1: LC-MS (ESI) m/z: 650 [M+H]⁺; ¹H-NMR (CD₃OD, 400 MHz): δ (ppm) 0.41-0.82 (m, 2H), 0.92 (t, J=7.2 Hz, 3H), 1.02-1.19 (m, 1H), 1.26 (d, J=6 Hz, 3H), 1.42-1.78 (m, 7H), 2.61-2.98 (m, 1H), 4.12-4.32 (m, 2H), 4.46-4.63 (m, 2H), 7.13-7.16 (m, 1H), 7.21-7.26 (m, 2H), 7.30-7.43 (m, 3H), 7.48-7.57 (m, 1H), 7.66-7.81 (m, 2H), 8.09-8.13 (m, 1H). Chiral separation condition: MeOH contained 0.1% DEA; IC 250×4.6 mm, 5 μm); retention time: 5.92 minute.

Compound 13-2: LC-MS (ESI) m/z: 650 [M+H]⁺; ¹H-NMR (CD₃OD, 400 MHz): δ (ppm) 0.45-0.82 (m, 2H), 0.92 (t, J=7.2 Hz, 3H), 1.02-1.16 (m, 1H), 1.26 (d, J=6 Hz, 3H), 1.40-1.75 (m, 7H), 2.71-2.98 (m, 1H), 4.09-4.32 (m, 2H), 4.56-4.61 (m, 2H), 7.15-7.18 (m, 1H), 7.21-7.26 (m, 2H), 7.30-7.43 (m, 3H), 7.46-7.58 (m, 1H), 7.66-7.81 (m, 2H), 8.09-8.13 (m, 1H). Chiral separation condition: MeOH contained 0.1% DEA; IC 250×4.6 mm, 5 μm); retention time: 4.76 minute.

Compound 13-3: LC-MS (ESI) m/z: 650 [M+H]⁺; ¹H-NMR (CD₃OD, 400 MHz): δ (ppm) 0.41-0.82 (m, 2H), 0.92 (t, J=7.2 Hz, 3H), 1.02-1.19 (m, 1H), 1.26 (d, J=6 Hz, 3H), 1.42-1.78 (m, 7H), 2.61-2.98 (m, 1H), 4.12-4.32 (m, 2H), 4.46-4.63 (m, 2H), 7.13-7.18 (m, 1H), 7.21-7.26 (m, 2H), 7.30-7.42 (m, 3H), 7.46-7.57 (m, 1H), 7.66-7.81 (m, 2H), 8.08-8.13 (m, 1H). Chiral separation condition: MeOH contained 0.1% DEA; IC 250×4.6 mm, 5 μm); retention time: 5.88 minute.

Compound 13-4: LC-MS (ESI) m/z: 650 [M+H]⁺; ¹H-NMR (CD₃OD, 400 MHz): δ (ppm) 0.41-0.82 (m, 2H), 0.92 (t, J=7.2 Hz, 3H), 1.02-1.19 (m, 1H), 1.26 (d, J=6 Hz, 3H), 1.42-1.78 (m, 7H), 2.61-3.01 (m, 1H), 4.12-4.41 (m, 2H), 4.49-4.64 (m, 2H), 7.13-7.16 (m, 1H), 7.21-7.26 (m, 2H), 7.30-7.43 (m, 3H), 7.48-7.57 (m, 1H), 7.66-7.81 (m, 2H), 8.08-8.11 (m, 1H). Chiral separation condition: MeOH contained 0.1% DEA; IC 250×4.6 mm, 5 μm); retention time: 4.76 minute.

### Example 14

### Synthesis of N-(3-(3-amino-8-azabicyclo[3.2.1]octan-8-yl)-1-(4-bromophenyl)-1,1l-difluoro-3-oxopropan-2-yl)-2-(5-(4-fluorophenyl)pyridin-2-yl)-2-oxoacetamide trifluoroacetate (Compound 14)

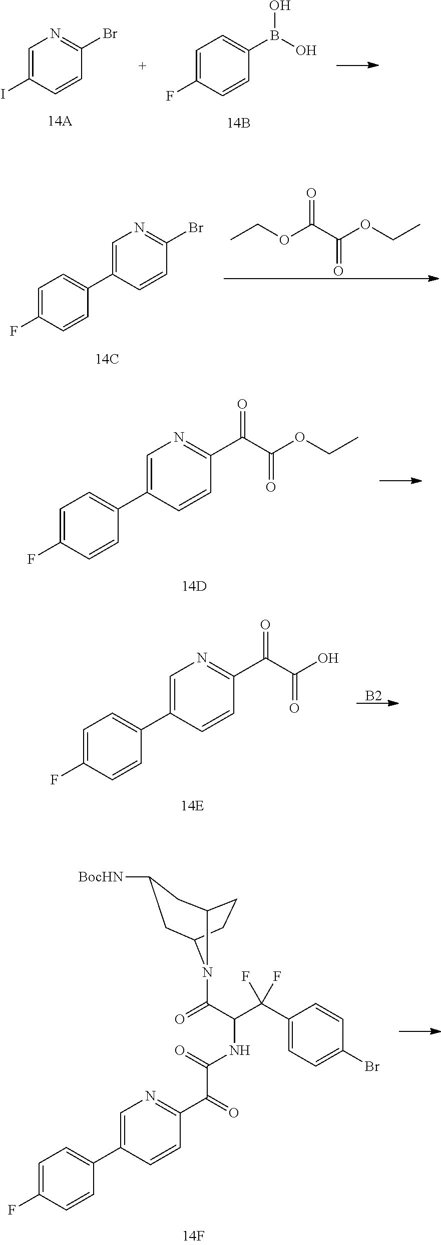

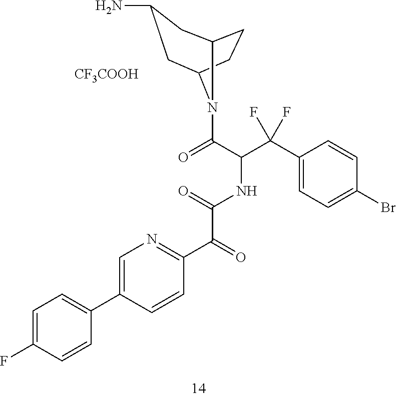 [SMILES: NC1CC2CCC(C1)N2C(=O)C(NC(=O)C(=O)c1ccc(-c2ccc(F)cc2)cn1)C(F)(F)c1ccc(Br)cc1 ; "CF3COOH"]

A mixture of Compound 14A (15 g, 58.3 mmol), Compound 14B (8.2 g, 58.3 mmol), Pd(dppf)Cl₂ (2.16 g, 2.65 mmol), and K₂CO₃ (21.9 g, 159 mmol) in 1,4-dioxane (150 mL) and water (90 mL) was heated at reflux under nitrogen for 5 hours. To the reaction mixture was added ethyl acetate (300 mL) and washed with water (250 mL), followed by saturated sodium bicarbonate solution (100 mL) and brine (100 mL). The organic layer was dried over anhydrous sodium sulfate, filtered, and concentrated to give a crude product. The crude product was purified by silica gel column chromatography to furnish Compound 14C. LC-MS (ESI) m/z: 252 [M+H]⁺; ¹H-NMR (DMSO-d₆, 400 MHz): δ (ppm) 7.36 (t, J=9.2 Hz, 2H), 7.74 (d, J=8.4 Hz, 1H), 7.79-7.82 (m, 2H), 8.04-8.07 (m, 1H), 8.71 (d, J=2.4 Hz, 1H).

Compounds 14D, 14E, 14F, and 14 were synthesized by employing the procedures described for Compounds 9B, 9C, 9D, and 1 using Compounds 14C, 14D, 14E, and 14F in lieu of Compounds 9A, 9B, 9C, and 1A.

Compound 14D: LC-MS (ESI) m/z: 274 [M+H]⁺; ¹H-NMR (DMSO-d₆, 400 MHz): δ (ppm) 1.34 (t, J=6.8 Hz, 3H), 4.43 (q, J=7.2 Hz, 2H), 7.39-7.45 (m, 2H), 7.93-7.98 (m, 2H), 8.21 (d, J=8.0 Hz, 1H), 8.42 (dd, J=8.0, 2.0 Hz, 1H), 9.15 (d, J=1.6 Hz, 1H).

Compound 14E: LC-MS (ESI) m/z: 246 [M+H]⁺; ¹H-NMR (DMSO-d₆, 400 MHz): δ (ppm) 7.38 (t, J=8.8 Hz, 2H), 7.86-7.89 (m, 2H), 7.95 (d, J=8.4 Hz, 1H), 8.23 (dd, J=8.4, 2.4 Hz, 1H), 8.97 (d, J=1.6 Hz, 1H).

Compound 14F: LC-MS (ESI) m/z: 715 [M+H]⁺.

Compound 14: LC-MS (ESI) m/z: 615 [M+H]⁺; ¹H-NMR (DMSO-d₆, 400 MHz): δ (ppm) 1.55-1.99 (m, 8H), 3.53-3.58 (m, 1H), 4.54-4.79 (m, 2H), 5.70-5.76 (m, 1H), 7.38-8.31 (m, 12H), 8.32 (d, J=8.4 Hz, 1H), 8.94 (s, 1H), 9.77-9.89 (m, 1H).

### Example 15

### Synthesis of N-((2R)-3-(3-amino-8-azabicyclo[3.2.1]octan-8-yl)-1-(4-bromophenyl)-1,1-difluoro-3-oxopropan-2-yl)-4-((4-chlorobenzyl)oxy)benzenesulfonamide trifluoroacetate (Compound 15-1), and N-((2S)-3-(3-amino-8-azabicyclo[3.2.1]octan-8-yl)-1-(4-bromophenyl)-1,1-difluoro-3-oxopropan-2-yl)-4-((4-chlorobenzyl)oxy) benzenesulfonamide trifluoroacetate (Compound 15-2)

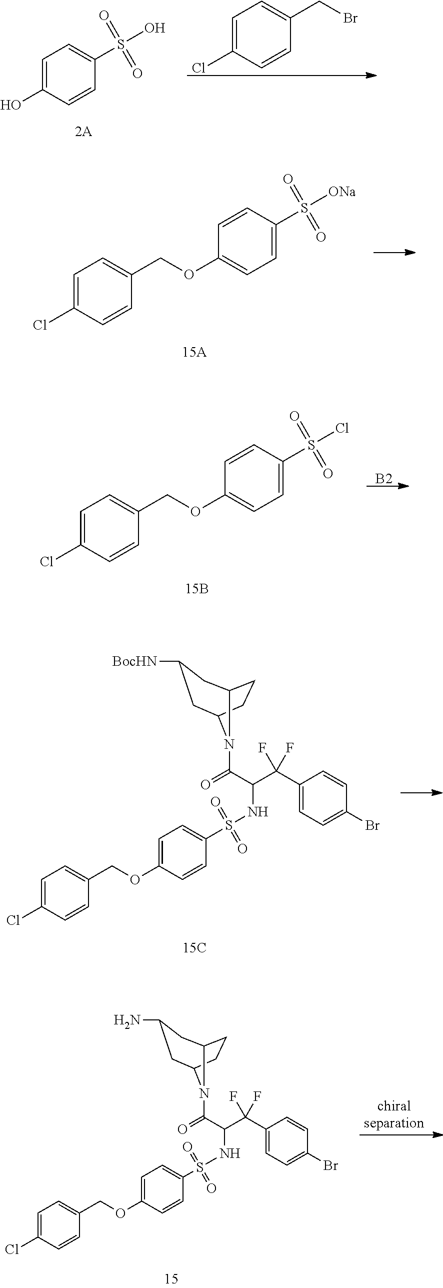

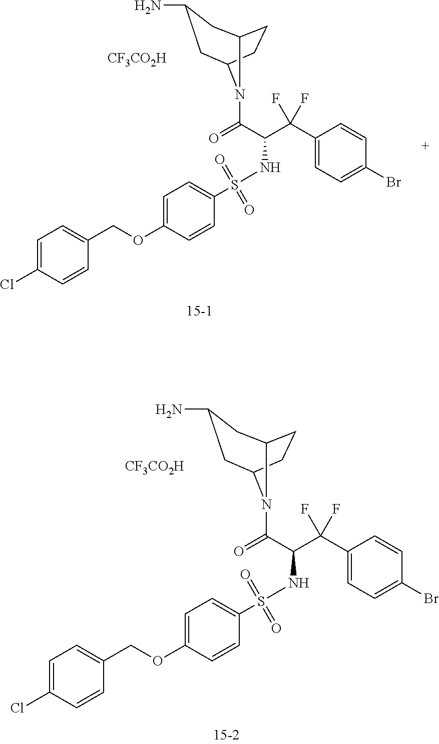 [SMILES: NC1CC2CCC(C1)N2C(=O)[C@@H](NS(=O)(=O)c1ccc(OCc2ccc(Cl)cc2)cc1)C(F)(F)c1ccc(Br)cc1 ; "CF3CO2H" ; NC1CC2CCC(C1)N2C(=O)[C@H](NS(=O)(=O)c1ccc(OCc2ccc(Cl)cc2)cc1)C(F)(F)c1ccc(Br)cc1 ; "CF3CO2H"]

Compounds 15A, 15B, 15C, and 15 were synthesized by employing the procedures described for Compound 2B, Compound A1, Compounds 2D, and 1 using 1-(bromomethyl)-4-chlorobenzene, Compounds 15A, 15B, and 15C in lieu of 3-(bromomethyl)pentane, Compound A1-3, Compounds 2C, and 1A.

Compound 15A: LC-MS (ESI) m/z: 297 [M−Na]⁺.

Compound 15B: ¹H-NMR (CDCl₃, 400 MHz): δ (ppm) 5.14 (s, 2H), 7.10 (d, J=9.2 Hz, 2H), 7.35-7.41 (m, 4H), 7.98 (d, J=8.8 Hz, 2H).

Compound 15C: LC-MS (ESI) m/z: 790 [M+Na]⁺.

Compound 15 was separated with chiral HPLC to afford Compound 15-1 and Compound 15-2.

Compound 15-1: LC-MS (ESI) m/z: 668 [M+H]⁺; ¹H-NMR (CD₃OD, 400 MHz): δ (ppm) 1.29-2.07 (m, 8H), 3.48-3.64 (m, 1H), 4.39-4.74 (m, 3H), 5.07-5.09 (m, 2H), 6.88-6.93 (m, 2H), 7.14-7.17 (m, 1H), 7.30-7.51 (m, 9H). Chiral separation condition: MeOH contained 0.1% DEA; OJ-H (4.6×250 mm, 5 μm); retention time: 4.64 minute.

Compound 15-2: LC-MS (ESI) m/z: 668 [M+H]⁺; ¹H-NMR (CD₃OD, 400 MHz): δ (ppm) 1.29-2.07 (m, 8H), 3.48-3.64 (m, 1H), 4.39-4.74 (m, 3H), 5.07-5.09 (m, 2H), 6.88-6.93 (m, 2H), 7.14-7.17 (m, 1H), 7.30-7.51 (m, 9H). Chiral separation condition: MeOH contained 0.1% DEA; OJ-H (4.6×250 mm, 5 μm); retention time: 2.82 minute.

### Example 16

### Synthesis of N-((2R)-3-(3-amino-8-azabicyclo[3.2.1]octan-8-yl)-1-(4-bromophenyl)-1,1-difluoro-3-oxopropan-2-yl)-6-isopropylnaphthalene-2-sulfonamide trifluoroacetate (Compound 16-1), and N-((2S)-3-(3-amino-8-azabicyclo[3.2.1]octan-8-yl)-1-(4-bromophenyl)-1,1-difluoro-3-oxopropan-2-yl)-6-isopropylnaphthalene-2-sulfonamide trifluoroacetate (Compound 16-2)

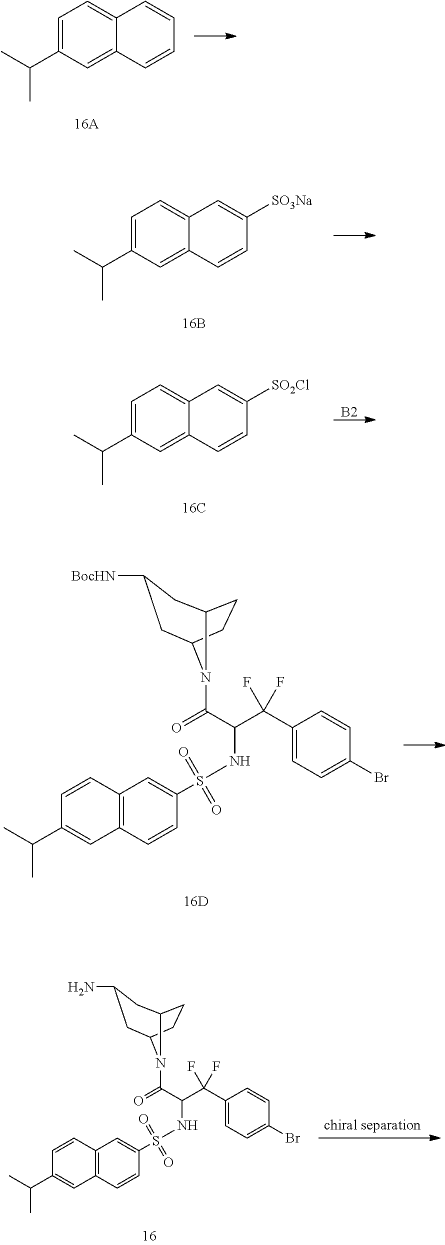

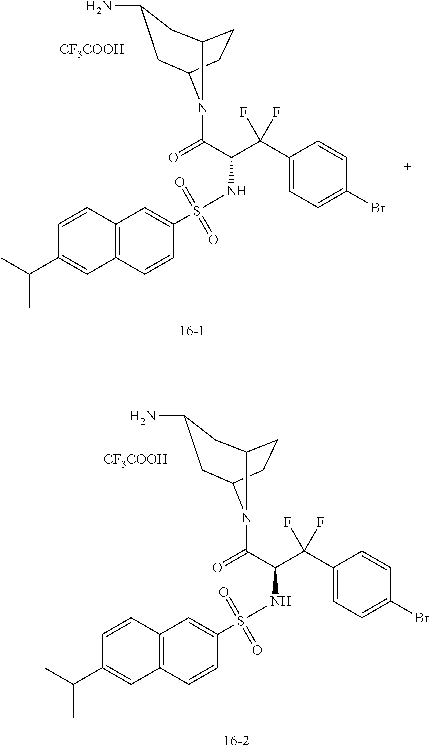 [SMILES: CC(C)c1ccc2cc(S(=O)(=O)N[C@H](C(=O)N3C4CCC3CC(N)C4)C(F)(F)c3ccc(Br)cc3)ccc2c1 ; "CF3COOH" ; CC(C)c1ccc2cc(S(=O)(=O)N[C@@H](C(=O)N3C4CCC3CC(N)C4)C(F)(F)c3ccc(Br)cc3)ccc2c1 ; "CF3COOH"]

To Compound 16A (1.0 g, 5.9 mmol) at 40° C. was slowly added concentrated H₂SO₄ (0.8 g, 8.8 mmol), which temperature could be raised to 140° C. during the addition. The reaction mixture was heated between 90-120° C. for additional 6 hours. The reaction mixture was cooled down to room temperature, poured into ice-cold water (10 mL), and purified with reverse phase chromatography using eluent (methanol in water, from 0% to 100% v/v) to give a crud acid product, which was dissolved in ethanol (20 mL). To the solution was added solid sodium hydroxide (0.5 g), stirred at 100° C. for 30 minutes, cooled down to room temperature, and filtered to afford Compound 16B. LC-MS (ESI) m/z: 249 [M−Na]⁺; ¹H-NMR (DMSO-d₆, 400 MHz): δ (ppm) 1.29 (s, 3H), 1.31 (s, 3H), 3.02-3.07 (m, 1H), 7.46 (d, J=8 Hz, 1H), 7.67 (d, J=8.4 Hz, 1H), 7.71 (s, 1H), 7.80 (d, J=8.8 Hz, 1H), 7.89 (d, J=8.4 Hz, 1H), 8.09 (s, 1H).

Compounds 16C, 16D, and 16 were synthesized by employing the procedures described for Compound A1, Compounds 2D, and 1 using Compounds 16B, 16C, and 16D in lieu of Compound A1-3, Compounds 2C, and 1A.

Compound 16C. LC-MS (ESI) m/z: 264 [M−Cl+CH₃NH₂]⁺; ¹H-NMR (CDCl₃, 400 MHz): δ (ppm) 1.36 (s, 3H), 1.38 (s, 3H), 3.12-3.15 (m, 1H), 7.58-7.61 (m, 1H), 7.76 (s, 1H), 7.96-7.98 (m, 3H), 8.56 (s, 1H).

Compound 16D. LC-MS (ESI) m/z: 720 [M+H]⁺.

Compound 16 was separated with chiral HPLC to furnish Compound 16-1 and Compound 16-2.

Compound 16-1: LC-MS (ESI) m/z: 620 [M+H]⁺; ¹H-NMR (CD₃OD, 400 MHz): δ (ppm) 0.62-2.09 (m, 14H), 3.03-3.05 (m, 1H), 3.41-3.63 (m, 1H), 4.23-4.62 (m, 3H), 7.04-7.09 (m, 2H), 7.29-7.51 (m, 4H), 7.68-7.80 (m, 3H), 8.04-8.13 (m, 1H). Chiral separation condition: MeOH contained 0.5% DEA; IC (4.6×250 mm, 5 μm); retention time: 5.00 minute.

Compound 16-2: LC-MS (ESI) m/z: 620 [M+H]⁺; ¹H-NMR (CD₃OD, 400 MHz): δ (ppm) 0.71-2.21 (m, 14H), 3.14-3.15 (m, 1H), 3.50-3.75 (m, 1H), 4.34-4.73 (m, 3H), 7.15-7.17 (m, 2H), 7.40-7.62 (m, 4H), 7.79-7.91 (m, 3H), 8.14-8.23 (m, 1H). Chiral separation condition: MeOH contained 0.5% DEA; IC (4.6×250 mm, 5 μm); retention time: 4.06 minute.

### Example 17

### Synthesis of N-((2R)-3-(3-amino-8-azabicyclo[3.2.1]octan-8-yl)-1-(4-bromophenyl)-1,1-difluoro-3-oxopropan-2-yl)-3-((4-fluorobenzyl)oxy)benzenesulfonamide trifluoroacetate (Compound 17-1), and N-((2S)-3-(3-amino-8-azabicyclo[3.2.1]octan-8-yl)-1-(4-bromophenyl)-1,1-difluoro-3-oxopropan-2-yl)-3-((4-fluorobenzyl)oxy) benzenesulfonamide trifluoroacetate (Compound 17-2)

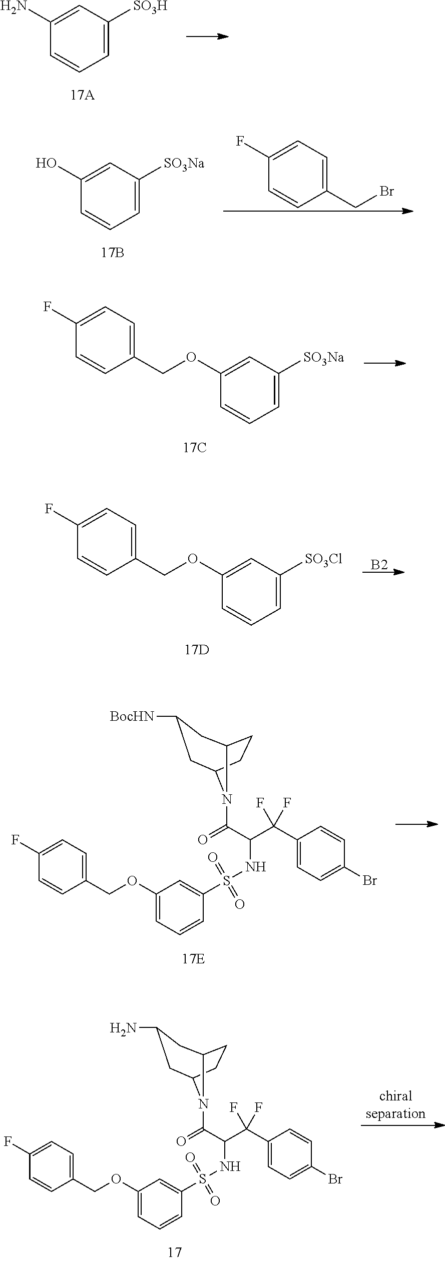

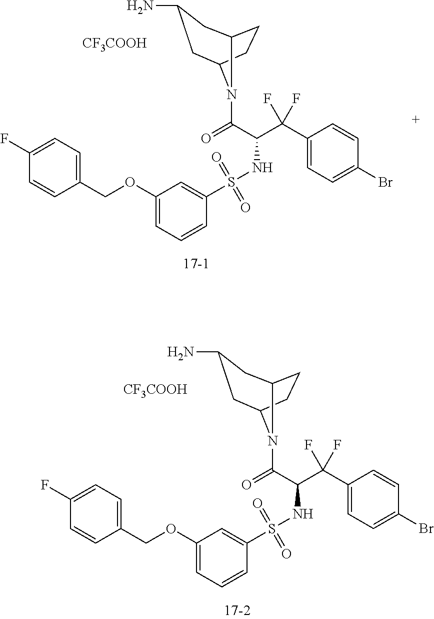 [SMILES: NC1CC2CCC(C1)N2C(=O)[C@@H](NS(=O)(=O)c1cccc(OCc2ccc(F)cc2)c1)C(F)(F)c1ccc(Br)cc1 ; "CF3COOH" ; NC1CC2CCC(C1)N2C(=O)[C@H](NS(=O)(=O)c1cccc(OCc2ccc(F)cc2)c1)C(F)(F)c1ccc(Br)cc1 ; "CF3COOH"]

To a solution of Compound 17A (5.0 g, 28.9 mmol) and sulfuric acid (8.6 g, 88 mmol) in water (100 mL) was added sodium nitrite (2.2 g, 32 mmol) in several small portions and stirred for 30 minutes. The reaction mixture was heated at reflux for 20 minutes and concentrated under reduced pressure to provide a residue. The residue was extracted with hot ethanol (100 mL×2). The combined organic extracts were concentrated and treated with solid sodium hydroxide until its pH showed basic and concentrated to provide a crude product Compound 17B. LC-MS (ESI) m/z: 174 [M−Na]⁺; ¹H-NMR (D₂O, 400 MHz): δ (ppm) 6.88 (s, 1H), 7.10-7.14 (m, 2H), 7.32 (d, J=6.4 Hz, 1H).

Compound 17C was synthesized by employing the procedure described for Compound 4D using 1-(bromomethyl)-4-fluorobenzene and Compound 17B in lieu of 2-iodopropane and Compound 4C. LC-MS (ESI) m/z: 281 [M−Na]⁺; ¹H-NMR (CD₃OD, 400 MHz): δ (ppm) 5.11 (s, 2H), 7.06-7.14 (m, 3H), 7.33-7.37 (m, 1H), 7.42-7.51 (m, 4H).

A mixture of Compound 17C (512 mg 1.7 mmol) and POCl₃ (5 mL) was refluxed for about 2 hours and concentrated. The residue was diluted with ethyl acetate (50 mL) and its pH was adjusted to about 7 with saturated sodium bicarbonate aqueous solution. The organic layer was separated, washed with water (50 mL) and brine (50 mL), dried over anhydrous sodium sulfate, filtered, and concentrated under reduced pressure to furnish a crude product Compound 17D, which was used for the next step without further purification. LC-MS (ESI) m/z: 296 [M+CH₃NH₂+H]⁺.

Compounds 17E and 17 were synthesized by employing the procedures described for Compounds 2D and 1 using Compounds 17D and 17E in lieu of Compound 2C and Compound 1A.

Compound 17E: LC-MS (ESI) m/z: 752 [M+H]⁺.

Compound 17 was separated with chiral HPLC to furnish Compound 17-1 and Compound 17-2.

Compound 17-1: LC-MS (ESI) m/z: 652 [M+H]⁺; ¹H-NMR (CD₃OD, 400 MHz): δ (ppm) 1.42-2.17 (m, 8H), 3.60-3.74 (m, 1H), 4.54-4.89 (m, 3H), 5.10-5.11 (m, 2H), 7.14-7.15 (m, 2H), 7.21-7.44 (m, 7H), 7.51-7.56 (m, 3H). Chiral separation condition: MeOH contained 0.1% DEA; OZ-H (4.6×250 mm, 5 μm); retention time: 5.73 minute.

Compound 17-2: LC-MS (ESI) m/z: 652 [M+H]⁺; ¹H-NMR (CD₃OD, 400 MHz): δ (ppm) 1.35-2.03 (m, 8H), 3.51-3.60 (m, 1H), 4.42-4.76 (m, 3H), 4.99-5.00 (m, 2H), 7.03-7.05 (m, 2H), 7.11-7.33 (m, 7H), 7.45-7.48 (m, 3H). Chiral separation condition: MeOH contained 0.1% DEA; OZ-H (4.6×250 mm, 5 μm); retention time: 4.23 minute.

### Example 18

### Synthesis of N-((2R)-3-(3-amino-8-azabicyclo[3.2.1]octan-8-yl)-1-(4-bromophenyl)-1,1-difluoro-3-oxopropan-2-yl)-4-((5-chloropyridin-2-yl)oxy)benzenesulfonamide trifluoroacetate (Compound 18-1), and N-((2S)-3-(3-amino-8-azabicyclo[3.2.1]octan-8-yl)-1-(4-bromophenyl)-1,1-difluoro-3-oxopropan-2-yl)-4-((5-chloropyridin-2-yl)oxy) benzenesulfonamide trifluoroacetate (Compound 18-2)

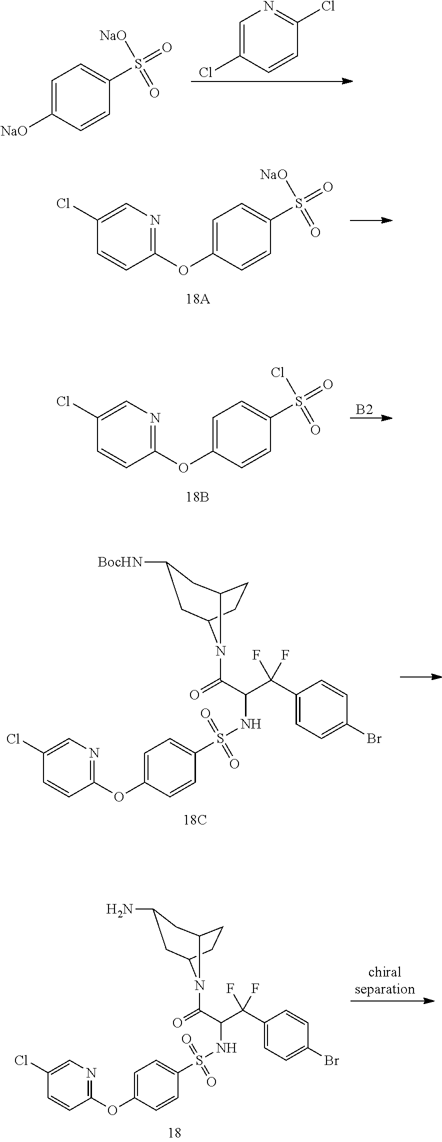

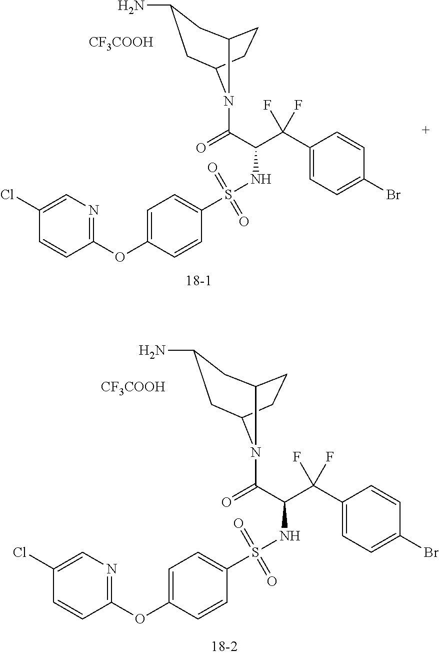 [SMILES: NC1CC2CCC(C1)N2C(=O)[C@@H](NS(=O)(=O)c1ccc(Oc2ccc(Cl)cn2)cc1)C(F)(F)c1ccc(Br)cc1 ; "CF3COOH" ; NC1CC2CCC(C1)N2C(=O)[C@H](NS(=O)(=O)c1ccc(Oc2ccc(Cl)cn2)cc1)C(F)(F)c1ccc(Br)cc1 ; "CF3COOH"]

Compounds 18A, 18B, 18C, and 18 were synthesized by employing the procedures described for Compounds A1-3, A1, Compounds 2D and 1 using sodium 4-oxidobenzenesulfonate, 2,5-dichloropyridine, Compounds 18A, 18B, and 18C in lieu of A1-2, 2-bromopropane, Compound A1-3, Compounds 2C, and 1A.

Compound 18A: LC-MS (ESI) m/z: 284 [M−Na]⁺.

Compound 18B: ¹H-NMR (CDCl₃, 400 MHz): δ (ppm) 7.03 (d, J=8.4 Hz, 1H), 7.34 (d, J=8.8 Hz, 2H), 7.75-7.78 (m, 1H), 8.06 (d, J=8.8 Hz, 2H), 8.17 (d, J=2.4 Hz, 1H).

Compound 18C: LC-MS (ESI) m/z: 777 [M+Na]⁺.

Compound 18 was separated with chiral HPLC to furnish Compound 18-1 and Compound 18-1.

Compound 18-1: LC-MS (ESI) m/z: 655 [M+H]⁺; ¹H-NMR (CD₃OD, 400 MHz): δ (ppm) 1.61-1.94 (m, 6H), 2.03-2.15 (m, 2H), 3.64-3.72 (m, 1H), 4.61-4.89 (m, 3H), 7.11-7.31 (m, 4H), 7.44-7.53 (m, 2H), 7.62-7.74 (m, 3H), 7.90-7.95 (m, 1H), 8.15-8.21 (m, 1H). Chiral separation condition: MeOH contained 0.5% DEA; IC (4.6×250 mm, 5 μm); retention time: 6.47 minute.

Compound 18-2: LC-MS (ESI) m/z: 655 [M+H]⁺; ¹H-NMR (CD₃OD, 400 MHz): δ (ppm) 1.61-1.94 (m, 6H), 2.03-2.15 (m, 2H), 3.65-3.72 (m, 1H), 4.60-4.89 (m, 3H), 7.11-7.31 (m, 4H), 7.44-7.53 (m, 2H), 7.62-7.74 (m, 3H), 7.90-7.95 (m, 1H), 8.15-8.21 (m, 1H). Chiral separation condition: MeOH contained 0.5% DEA; IC (4.6×250 mm, 5 μm); retention time: 5.11 minute.

### Example 19

### Synthesis of (2S)—N-((2R)-3-(3-amino-8-azabicyclo[3.2.1]octan-8-yl)-1-(4-bromophenyl)-1,1-difluoro-3-oxopropan-2-yl)-2-hydroxy-2-(6-isopropoxy naphthalen-2-yl)acetamide trifluoroacetate (Compound 19-3), (2R)—N-((2S)-3-(3-amino-8-azabicyclo[3.2.1]octan-8-yl)-1-(4-bromophenyl)-1,1-difluoro-3-oxopropan-2-yl)-2-hydroxy-2-(6-isopropoxynaphthalen-2-yl)acetamide trifluoroacetate (Compound 19-4), (2R)—N-((2R)-3-(3-amino-8-azabicyclo[3.2.1]octan-8-yl)-1-(4-bromophenyl)-1,1-difluoro-3-oxopropan-2-yl)-2-hydroxy-2-(6-isopropoxynaphthalen-2-yl)acetamide trifluoroacetate (Compound 19-5), and (2S)—N-((2S)-3-(3-amino-8-azabicyclo[3.2.1]octan-8-yl)-1-(4-bromophenyl)-1,1-difluoro-3-oxopropan-2-yl)-2-hydroxy-2-(6-isopropoxynaphthalen-2-yl)acetamide trifluoroacetate (Compound 19-6)

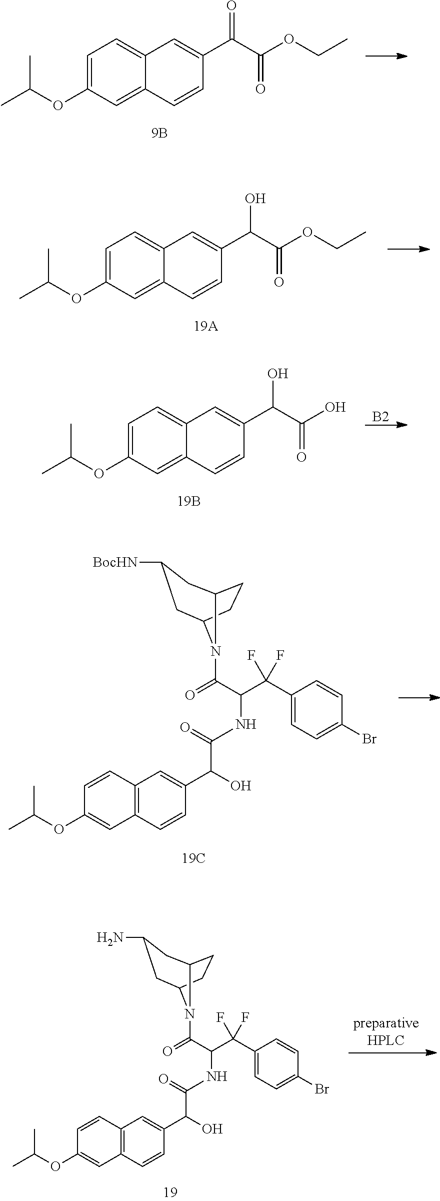

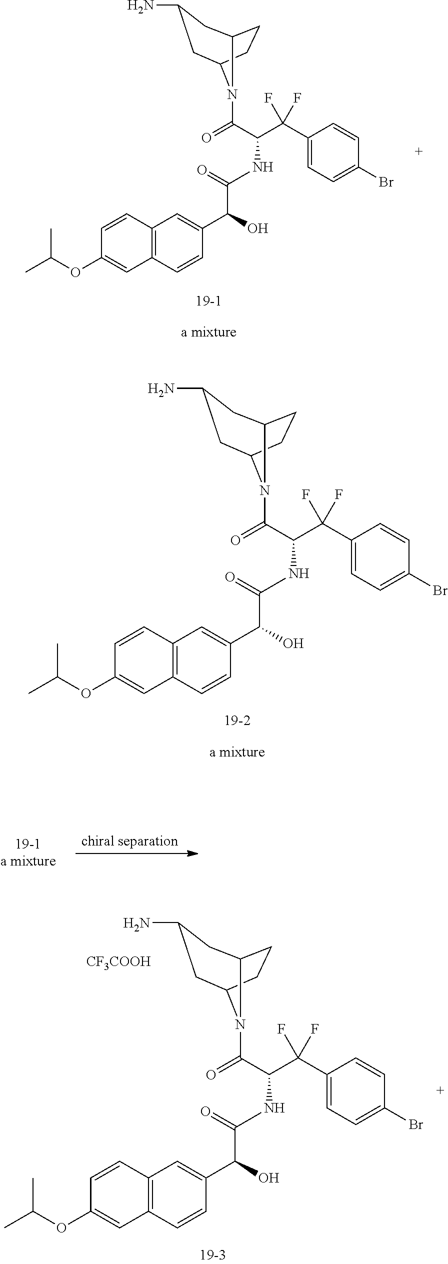 [SMILES: CC(C)Oc1ccc2cc([C@H](O)C(=O)N[C@H](C(=O)N3C4CCC3CC(N)C4)C(F)(F)c3ccc(Br)cc3)ccc2c1 ; CC(C)Oc1ccc2cc([C@@H](O)C(=O)N[C@H](C(=O)N3C4CCC3CC(N)C4)C(F)(F)c3ccc(Br)cc3)ccc2c1 ; CC(C)Oc1ccc2cc([C@H](O)C(=O)N[C@H](C(=O)N3C4CCC3CC(N)C4)C(F)(F)c3ccc(Br)cc3)ccc2c1 ; "CF3COOH"]

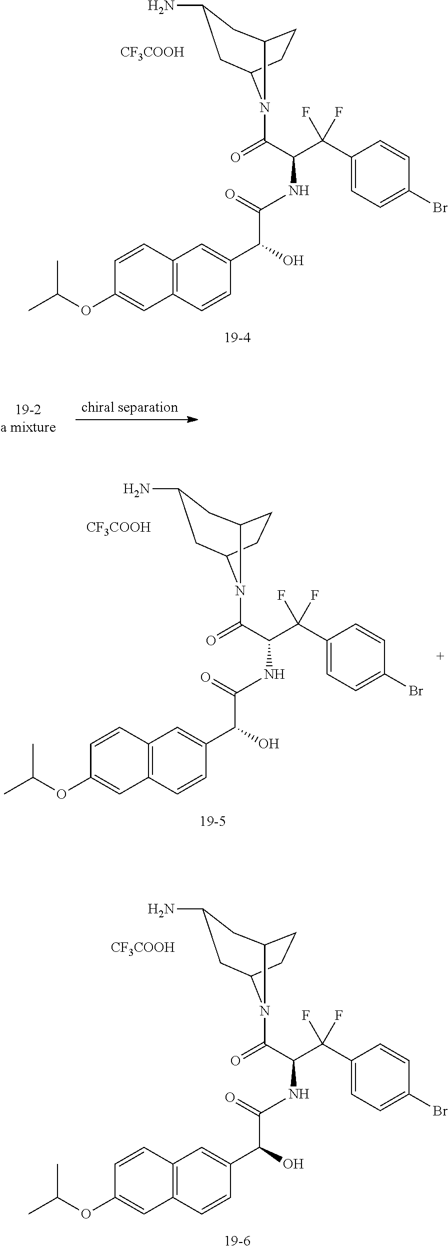 [SMILES: CC(C)Oc1ccc2cc([C@@H](O)C(=O)N[C@@H](C(=O)N3C4CCC3CC(N)C4)C(F)(F)c3ccc(Br)cc3)ccc2c1 ; "CF3COOH" ; CC(C)Oc1ccc2cc([C@@H](O)C(=O)N[C@H](C(=O)N3C4CCC3CC(N)C4)C(F)(F)c3ccc(Br)cc3)ccc2c1 ; "CF3COOH" ; CC(C)Oc1ccc2cc([C@H](O)C(=O)N[C@@H](C(=O)N3C4CCC3CC(N)C4)C(F)(F)c3ccc(Br)cc3)ccc2c1 ; "CF3COOH"]

To a solution of Compound 9B (286 mg, 1.0 mmol) in ethanol (10 mL) was added zinc powder (130 mg, 2.0 mmol), formic acid (92 mg, 3.0 mmol), and water (3 mL) and stirred at 10° C. for 10 hours. The reaction mixture was filtered and the filtrate was concentrated to furnish Compound 19A. LC-MS (ESI) m/z: 271 [M−OH].

Compounds 19B, 19C, and 19 were synthesized by employing the procedures described for Compounds 9C, 9D, and 1 using Compounds 19A, 19B using DMF as solvent, and 19C in lieu of Compounds 9B, 9C using dichloromethane as solvent, and 1A.

Compound 19B: LC-MS (ESI) m/z: 243 [M−OH]⁺; ¹H-NMR (DMSO-d₆, 400 MHz): δ (ppm) 1.32 (d, J=6.0 Hz, 6H), 4.73-4.76 (m, 1H), 4.98 (s, 1H), 7.09-7.11 (m, 1H), 7.28 (s, 1H), 7.47-7.49 (m, 1H), 7.70-7.82 (m, 3H).

Compound 19C: LC-MS (ESI) m/z: 752 [M+Na]⁺.

Compound 19 was separated with preparative HPLC to give two enatiomeric mixtures of Compound 19-1 and Compound 19-2, which was respectively separated with chiral HPLC to give Compound 19-3, Compound 19-4, Compound 19-5, and Compound 19-6.

Compound 19-3: LC-MS (ESI) m/z: 630 [M+H]⁺; ¹H-NMR (CD₃OD, 400 MHz): δ (ppm) 1.28 (d, J=6.0 Hz, 6H), 1.46-1.87 (m, 9H), 3.49-3.51 (m, 1H), 4.29-4.31 (m, 1H), 4.51-4.53 (m, 1H), 4.64-4.68 (m, 1H), 5.14 (s, 1H), 5.32-5.38 (m, 1H), 7.01-7.04 (m, 1H), 7.12-7.17 (m, 2H), 7.23-7.26 (m, 2H), 7.32-7.37 (m, 2H), 7.56-7.65 (m, 2H), 7.70 (s, 1H). Chiral separation condition: EtOH contained 0.5% DEA; OZ-H (4.6×250 mm, 5 μm); retention time: 3.23 minute.

Compound 19-4: LC-MS (ESI) m/z: 630 [M+H]⁺; ¹H-NMR (CD₃OD, 400 MHz): δ (ppm) 1.38 (d, J=6.0 Hz, 6H), 1.39-1.98 (m, 9H), 3.60-3.61 (m, 1H), 4.40-4.41 (m, 1H), 4.62-4.64 (m, 1H), 4.75-4.79 (m, 1H), 5.25 (s, 1H), 5.43-5.49 (m, 1H), 7.12-7.15 (m, 1H), 7.23-7.28 (m, 2H), 7.34-7.37 (m, 2H), 7.43-7.48 (m, 2H), 7.70-7.76 (m, 2H), 7.80 (s, 1H). Chiral separation condition: EtOH contained 0.5% DEA; OZ-H (4.6×250 mm, 5 μm); retention time: 4.56 minute.

Compound 19-5: LC-MS (ESI) m/z: 630 [M+H]⁺; ¹H-NMR (CD₃OD, 400 MHz): δ (ppm) 1.29 (d, J=6.0 Hz, 6H), 1.56-1.78 (m, 6H), 1.88-1.93 (m, 3H), 3.12-3.14 (m, 1H), 3.54-3.56 (m, 1H), 4.37-4.39 (m, 1H), 4.55-4.57 (m, 1H), 4.65-4.68 (m, 1H), 5.03 (s, 1H), 5.37-5.43 (m, 1H), 7.02-7.18 (m, 4H), 7.23-7.29 (m, 3H), 7.57-7.65 (m, 3H). Chiral separation condition: MeOH contained 0.5% DEA; IC (4.6×250 mm, 5 μm); retention time: 5.87 minute.

Compound 19-6: LC-MS (ESI) m/z: 630 [M+H]⁺; ¹H-NMR (CD₃OD, 400 MHz): δ (ppm) 1.38 (d, J=6.0 Hz, 6H), 1.67-1.88 (m, 6H), 2.02-2.05 (m, 3H), 3.65-3.38 (m, 1H), 4.48-4.49 (m, 1H), 4.66-4.67 (m, 1H), 4.75-4.78 (m, 1H), 5.14 (s, 1H), 5.48-5.54 (m, 1H), 7.13-7.29 (m, 4H), 7.34-7.39 (m, 3H), 7.65-7.76 (m, 3H). Chiral separation condition: MeOH contained 0.5% DEA; IC (4.6×250 mm, 5 μm); retention time: 6.96 minute.

### Example 20

### Synthesis of N-((2R)-3-(3-amino-8-azabicyclo[3.2.1]octan-8-yl)-1-(4-bromophenyl)-1,1-difluoro-3-oxopropan-2-yl)-4-(cyclopentylmethoxy)benzenesulfonamide trifluoroacetate (Compound 20-1), and N-((2S)-3-(3-amino-8-azabicyclo[3.2.1]octan-8-yl)-1-(4-bromophenyl)-1,1-difluoro-3-oxopropan-2-yl)-4-(cyclopentylmethoxy) benzenesulfonamide trifluoroacetate (Compound 20-2)

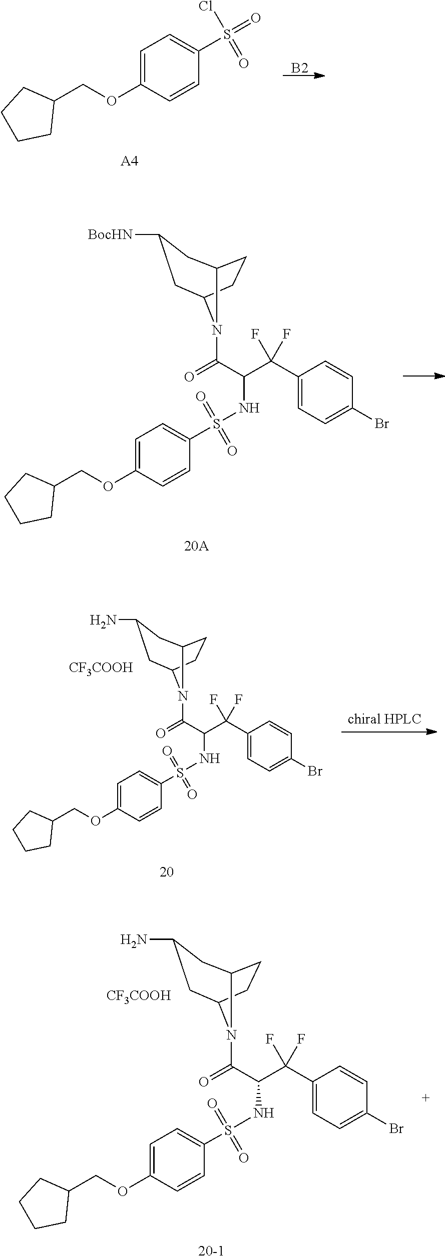

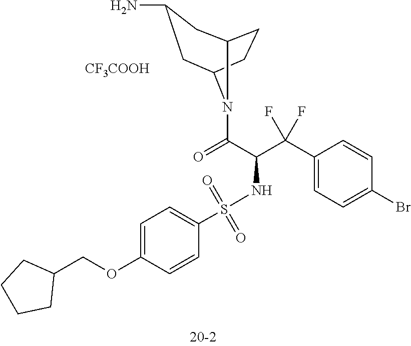 [SMILES: NC1CC2CCC(C1)N2C(=O)[C@H](NS(=O)(=O)c1ccc(OCC2CCCC2)cc1)C(F)(F)c1ccc(Br)cc1 ; "CF3COOH"]

Compounds 20A and 20 were synthesized by employing the procedures described for Compounds 2D and 6 using Compound A4 and Compound 20A in lieu of Compounds 2C and 6C.

Compound 20A: LC-MS (ESI) m/z: 726 [M+H]⁺.

Compound 20 was separated with chiral HPLC to give Compound 20-1 and Compound 20-2.

Compound 20-1: LC-MS (ESI) m/z: 626 [M+H]⁺; ¹H-NMR (CD₃OD, 400 MHz): δ (ppm) 1.40-2.45 (m, 17H), 3.64-3.71 (m, 1H), 3.92-3.98 (m, 2H), 4.60-4.92 (m, 3H), 6.88-6.95 (m, 2H), 7.25-7.27 (m, 1H), 7.44-7.70 (m, 5H). Chiral separation condition: n-hexane/EtOH contained 0.1% DEA, 70/30; (S,S)-WHELK-O1 (4.6×250 mm, 5 μm); retention time: 14.10 minute.

Compound 20-2: LC-MS (ESI) m/z: 626 [M+H]⁺; ¹H-NMR (CD₃OD, 400 MHz): δ (ppm) 1.41-2.43 (m, 17H), 3.64-3.71 (m, 1H), 3.92-3.98 (m, 2H), 4.60-4.92 (m, 3H), 6.88-6.95 (m, 2H), 7.25-7.27 (m, 1H), 7.44-7.70 (m, 5H). Chiral separation condition: n-hexane/EtOH contained 0.1% DEA, 70/30; (S,S)-WHELK-O1 (4.6×250 mm, 5 μm); retention time: 12.17 minute.

### Example 21

### Synthesis of N-((2R)-1-(4-bromophenyl)-3-(3-(dimethylamino)-8-azabicyclo[3.2.1]octan-8-yl)-1,1-difluoro-3-oxopropan-2-yl)-6-isopropoxynaphthalene-2-sulfonamide (Compound 21-1), and N-((2S)-1-(4-bromophenyl)-3-(3-(dimethylamino)-8-azabicyclo[3.2.1]octan-8-yl)-1,1-difluoro-3-oxopropan-2-yl)-6-isopropoxy naphthalene-2-sulfonamide (Compound 21-2)

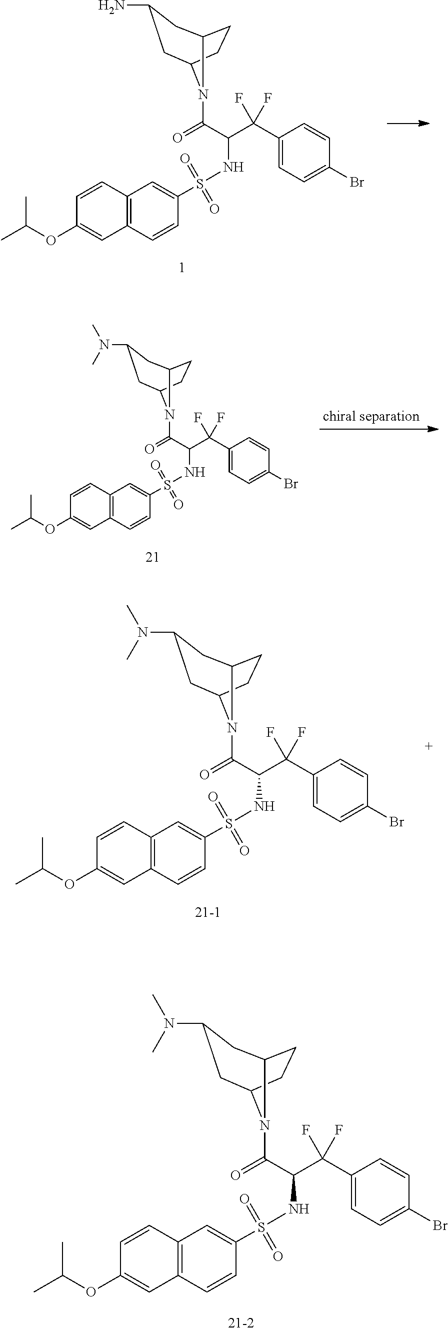 [SMILES: CC(C)Oc1ccc2cc(S(=O)(=O)NC(C(=O)N3C4CCC3CC(N)C4)C(F)(F)c3ccc(Br)cc3)ccc2c1 ; CC(C)Oc1ccc2cc(S(=O)(=O)NC(C(=O)N3C4CCC3CC(N(C)C)C4)C(F)(F)c3ccc(Br)cc3)ccc2c1 ; CC(C)Oc1ccc2cc(S(=O)(=O)N[C@H](C(=O)N3C4CCC3CC(N(C)C)C4)C(F)(F)c3ccc(Br)cc3)ccc2c1 ; CC(C)Oc1ccc2cc(S(=O)(=O)N[C@@H](C(=O)N3C4CCC3CC(N(C)C)C4)C(F)(F)c3ccc(Br)cc3)ccc2c1]

To a solution of Compound 1 (50 mg, 0.08 mmol) in methanol (8 mL) was added NaBH₃CN (15 mg, 0.24 mmol), paraformaldehyde (12 mg, 0.40 mmol), and acetic acid (1 drop). The reaction mixture was stirred at 50° C. overnight and concentrated under reduced pressure. The residue was purified with preparative HPLC and chiral HPLC to furnish Compound 21-1 and Compound 21-2.

Compound 21-1: LC-MS (ESI) m/z: 664 [M+H]⁺; ¹H-NMR (CD₃OD, 400 MHz): δ (ppm) 1.40-1.45 (m, 7H), 1.59-2.17 (m, 7H), 2.80-2.84 (m, 6H), 3.59-3.86 (m, 1H), 4.43-4.60 (m, 1H), 4.68-4.85 (m, 3H), 7.21 (s, 2H), 7.24-7.27 (m, 1H), 7.34 (s, 1H), 7.42-7.60 (m, 3H), 7.70-7.89 (m, 2H), 8.10-8.19 (m, 1H). Chiral separation condition: MeOH contained 0.1% DEA; (R,R)-whelk-ol (4.6×250 mm, 5 μm); retention time: 7.55 minute.

Compound 21-2: LC-MS (ESI) m/z: 664 [M+H]⁺; ¹H-NMR (CD₃OD, 400 MHz): δ (ppm) 1.40-1.45 (m, 7H), 1.58-2.17 (m, 7H), 2.80-2.84 (m, 6H), 3.59-3.87 (m, 1H), 4.43-4.60 (m, 1H), 4.67-4.86 (m, 3H), 7.21 (s, 2H), 7.23-7.27 (m, 1H), 7.34 (s, 1H), 7.42-7.60 (m, 3H), 7.70-7.88 (m, 2H), 8.10-8.19 (m, 1H). Chiral separation condition: MeOH contained 0.1% DEA; (R,R)-whelk-ol (4.6×250 mm, 5 μm); retention time: 11.15 minute.

### Example 22

### Synthesis of N-((2R)-3-(3-amino-8-azabicyclo[3.2.1]octan-8-yl)-1-(4-bromophenyl)-1,1-difluoro-3-oxopropan-2-yl)-4-butoxybenzenesulfonamide (Compound 22-1), and N-((2S)-3-(3-amino-8-azabicyclo[3.2.1]octan-8-yl)-1-(4-bromophenyl)-1,1-difluoro-3-oxopropan-2-yl)-4-butoxybenzenesulfonamide (Compound 22-2)

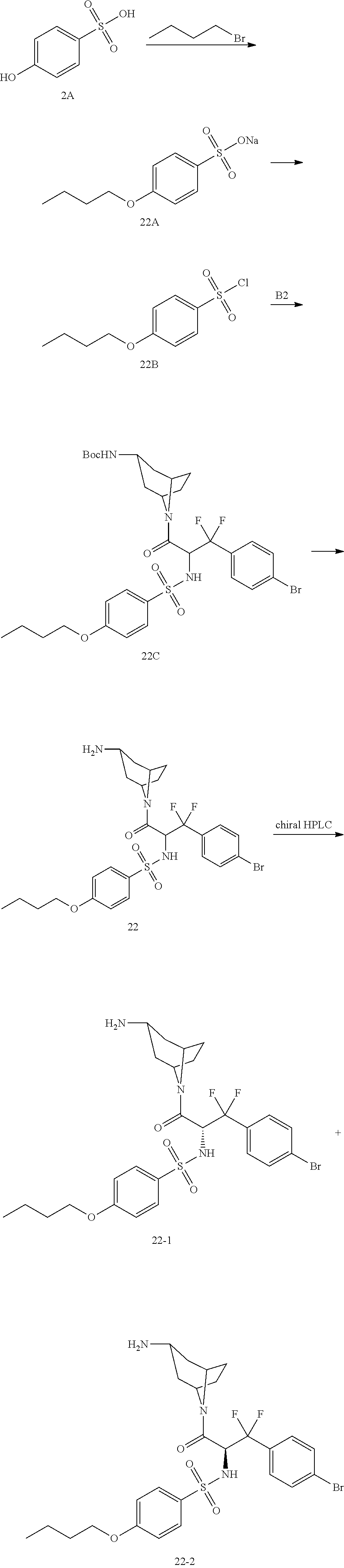

Compounds 22A, 22B, 22C, and 22 were synthesized by employing the procedures described for Compound 2B, Compound A1, Compounds 2D, and 1 using 1-bromobutane, Compounds 22A, 22B, and 22C in lieu of 3-(bromomethyl)pentane, Compound A1-3, Compounds 2C, and 1A.

Compound 22A: LC-MS (ESI) m/z: 229 [M−Na]⁺; ¹H-NMR (DMSO-d₆, 400 MHz): δ (ppm) 0.99 (t, J=8.0 Hz, 3H), 1.49-1.54 (m, 2H), 1.75-1.79 (m, 2H), 4.01 (t, J=6.8 Hz, 2H), 6.93 (d, J=8.8 Hz, 2H), 7.75 (d, J=8.8 Hz, 2H).

Compound 22B: LC-MS (ESI) m/z: 244 [M−Cl+CH₃NH₂]⁺.

Compound 22C: LC-MS (ESI) m/z: 700 [M+H]⁺.

Compound 22 was separated with chiral HPLC to afford Compound 22-1 and Compound 22-2.

Compound 22-1: LC-MS (ESI) m/z: 600 [M+H]⁺; ¹H-NMR (CD₃OD, 400 MHz): δ (ppm) 0.98-1.04 (m, 3H), 1.51-2.18 (m, 12H), 3.60-3.76 (m, 1H), 4.06-4.11 (m, 2H), 4.61-4.83 (m, 3H), 6.88-6.95 (m, 2H), 7.26 (d, J=8.8 Hz, 1H), 7.43 (t, J=8.0 Hz, 2H), 7.50 (d, J=8.0 Hz, 1H), 7.58 (d, J=8.0 Hz, 2H). Chiral separation condition: MeOH contained 0.5% DEA; IC (4.6×250 mm, 5 μm); retention time: 6.31 minute.

Compound 22-2: LC-MS (ESI) m/z: 600 [M+H]⁺; ¹H-NMR (CD₃OD, 400 MHz): δ (ppm) 0.98-1.04 (m, 3H), 1.50-2.17 (m, 12H), 3.61-3.74 (m, 1H), 4.04-4.11 (m, 2H), 4.57-4.92 (m, 3H), 6.89-6.95 (m, 2H), 7.26 (d, J=8.8 Hz, 1H), 7.43 (t, J=8.0 Hz, 2H), 7.50 (d, J=8.0 Hz, 1H), 7.58 (d, J=8.0 Hz, 2H). Chiral separation condition: MeOH contained 0.5% DEA; IC (4.6×250 mm, 5 μm); retention time: 5.14 minute.

### Example 23

### Synthesis of N-((2R)-3-(3-amino-8-azabicyclo[3.2.1]octan-8-yl)-1-(4-bromophenyl)-1,1-difluoro-3-oxopropan-2-yl)-4-(hexyloxy)benzenesulfonamide (Compound 23-1), and N-((2S)-3-(3-amino-8-azabicyclo[3.2.1]octan-8-yl)-1-(4-bromophenyl)-1,1-difluoro-3-oxopropan-2-yl)-4-(hexyloxy)benzenesulfonamide (Compound 23-2)

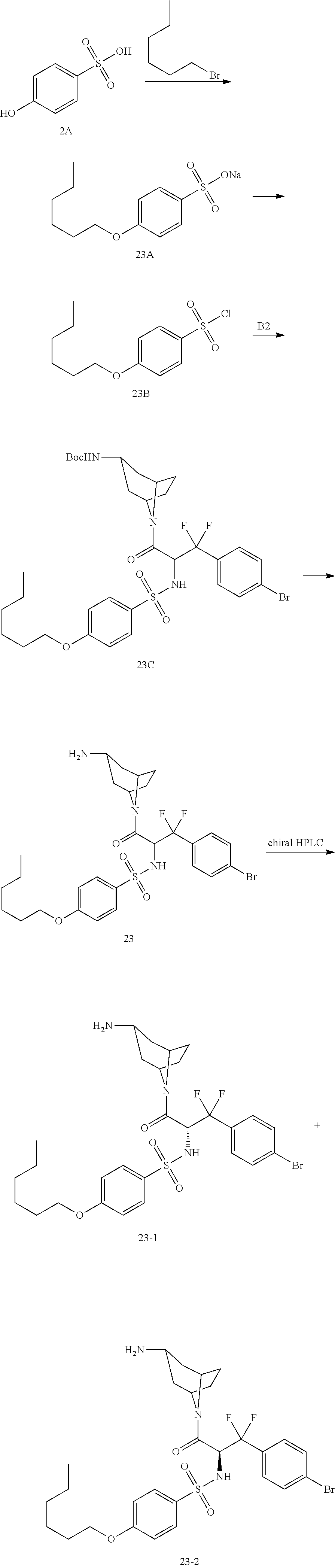

Compounds 23A, 23B, 23C, and 23 were synthesized by employing the procedures described for Compound 2B, Compound A1, Compounds 2D, and 1 using 1-bromohexane, Compounds 23A, 23B, and 23C in lieu of 3-(bromomethyl)pentane, Compound A1-3, Compounds 2C, and 1A.

Compound 23A: LC-MS (ESI) m/z: 257 [M−Na]⁺; ¹H-NMR (DMSO-d₆, 400 MHz): δ (ppm) 0.91-0.95 (m, 3H), 1.35-1.51 (m, 6H), 1.76-1.80 (m, 2H), 4.01 (t, J=6.4 Hz, 2H), 6.92-6.94 (m, 2H), 7.73-7.75 (m, 2H).

Compound 23B: LC-MS (ESI) m/z: 272 [M−Cl+CH₃NH+H]⁺.

Compound 23C: LC-MS (ESI) m/z: 728 [M+H]⁺.

Compound 23 was separated with chiral HPLC to afford Compound 23-1 and Compound 23-2.

Compound 23-1: LC-MS (ESI) m/z: 628 [M+H]⁺; ¹H-NMR (CD₃OD, 400 MHz): δ (ppm) 0.98-1.04 (m, 3H), 1.37-2.18 (m, 16H), 3.60-3.76 (m, 1H), 4.05-4.08 (m, 2H), 4.57-4.92 (m, 3H), 6.88-6.95 (m, 2H), 7.26 (d, J=8.8 Hz, 1H), 7.45 (t, J=8.0 Hz, 2H), 7.50 (d, J=8.0 Hz, 1H), 7.58 (d, J=8.0 Hz, 2H). Chiral separation condition: MeOH contained 0.5% DEA; IC (4.6×250 mm, 5 μm); retention time: 6.96 minute.

Compound 23-2: LC-MS (ESI) m/z: 628 [M+H]⁺; ¹H-NMR (CD₃OD, 400 MHz): δ (ppm) 0.94-0.96 (m, 3H), 1.37-2.18 (m, 16H), 3.62-3.75 (m, 1H), 4.03-4.10 (m, 2H), 4.61-4.82 (m, 3H), 6.88-6.95 (m, 2H), 7.26 (d, J=8.8 Hz, 1H), 7.43 (t, J=8.0 Hz, 2H), 7.50 (d, J=8.0 Hz, 1H), 7.58 (d, J=8.0 Hz, 2H). Chiral separation condition: MeOH contained 0.5% DEA; IC (4.6×250 mm, 5 m); retention time: 5.39 minute.

### Example 24

### Synthesis of N-((2R)-3-(3-amino-8-azabicyclo[3.2.1]octan-8-yl)-1-(4-bromophenyl)-1,1-difluoro-3-oxopropan-2-yl)-6-(benzyloxy)naphthalene-2-sulfonamide (Compound 24-1), and N-((2S)-3-(3-amino-8-azabicyclo[3.2.1]octan-8-yl)-1-(4-bromophenyl)-1,1-difluoro-3-oxopropan-2-yl)-6-(benzyloxy)naphthalene-2-sulfonamide (Compound 24-2)

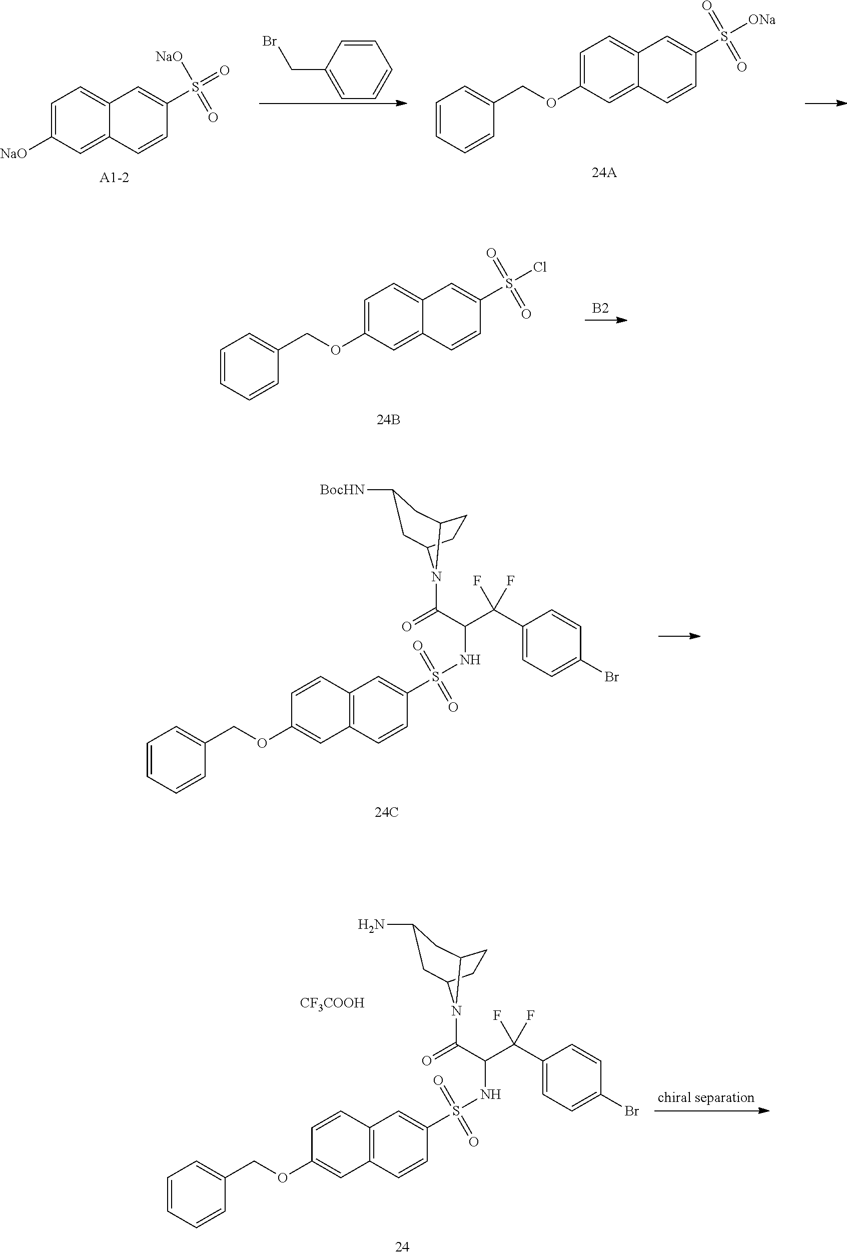

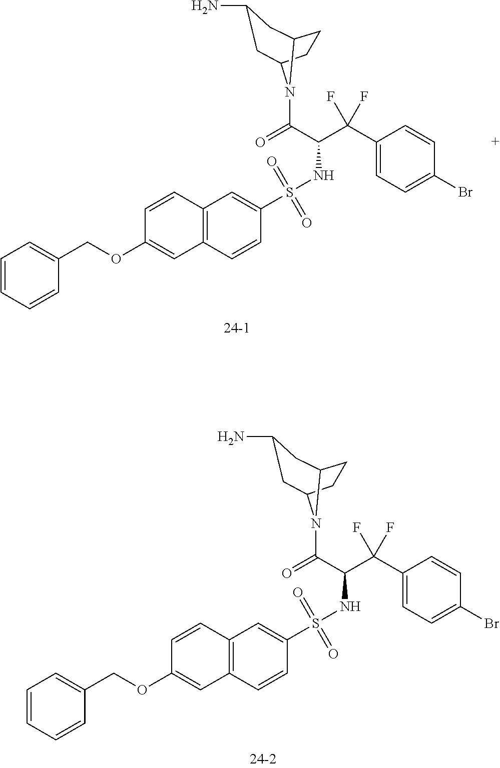 [SMILES: NC1CC2CCC(C1)N2C(=O)[C@@H](NS(=O)(=O)c1ccc2cc(OCc3ccccc3)ccc2c1)C(F)(F)c1ccc(Br)cc1 ; NC1CC2CCC(C1)N2C(=O)[C@H](NS(=O)(=O)c1ccc2cc(OCc3ccccc3)ccc2c1)C(F)(F)c1ccc(Br)cc1]

Compounds 24A, 24B, 24C, and 24 were synthesized by employing the procedures described for Compounds A1-3, A1, Compounds 2D, and 1 using benzyl bromide, Compounds 24A, 24B, and 24C in lieu of 2-bromopropane, Compound A1-3, Compounds 2C, and 1A.

Compound 24A: LC-MS (ESI) m/z: 315 [M+H]⁺.

Compound 24B: LC-MS (ESI) m/z: non-ionizable compound under routine conditions used.

Compound 24C, which was used directly for the next step without further purification. LC-MS (ESI) m/z: 806 [M+Na]⁺.

Compound 24, which was separated with chiral HPLC (MeOH contained 0.1% DEA; IC, 250×4.6 mm, 5 m) to give Compound 24-1 and Compound 24-2.

Compound 24-1: LC-MS (ESI) m/z: 684 [M+H]⁺; ¹H-NMR (CD₃OD, 400 MHz): δ (ppm) 0.79-1.14 (m, 2H), 1.35-1.51 (m, 1H), 1.54-2.13 (m, 5H), 3.00-3.11 (m, 1H), 4.02-4.46 (m, 2H), 4.51-4.62 (m, 1H), 5.15 (s, 2H), 7.17-7.32 (m, 8H), 7.40-7.42 (m, 3H), 7.48-7.51 (m, 2H), 7.67-7.81 (m, 2H), 8.10-8.11 (m, 1H). Chiral separation condition: MeOH contained 0.1% DEA; IC (4.6×250 mm, 5 μm); retention time: 7.22 minute.

Compound 24-2: LC-MS (ESI) m/z: 684 [M+H]⁺; ¹H-NMR (CD₃OD, 400 MHz): δ (ppm) 0.70-1.15 (m, 2H), 1.30-1.47 (m, 1H), 1.50-1.97 (m, 5H), 2.97-3.14 (m, 1H), 4.12-4.46 (m, 2H), 4.51-4.69 (m, 1H), 5.15 (s, 2H), 7.20-7.34 (m, 8H), 7.40-7.42 (m, 3H), 7.49-7.53 (m, 2H), 7.67-7.83 (m, 2H), 8.11-8.12 (m, 1H). Chiral separation condition: MeOH contained 0.1% DEA; IC (4.6×250 mm, 5 μm); retention time: 5.73 minute.

### Example 25

### Synthesis of N-((2R)-3-(3-amino-8-azabicyclo[3.2.1]octan-8-yl)-1-(4-bromophenyl)-1,1-difluoro-3-oxopropan-2-yl)-4-(4-fluorophenoxy)benzenesulfonamide trifluoroacetate (Compound 25-1), and N-((2S)-3-(3-amino-8-azabicyclo[3.2.1]octan-8-yl)-1-(4-bromophenyl)-1,1-difluoro-3-oxopropan-2-yl)-4-(4-fluorophenoxy) benzenesulfonamide trifluoroacetate (Compound 25-2)

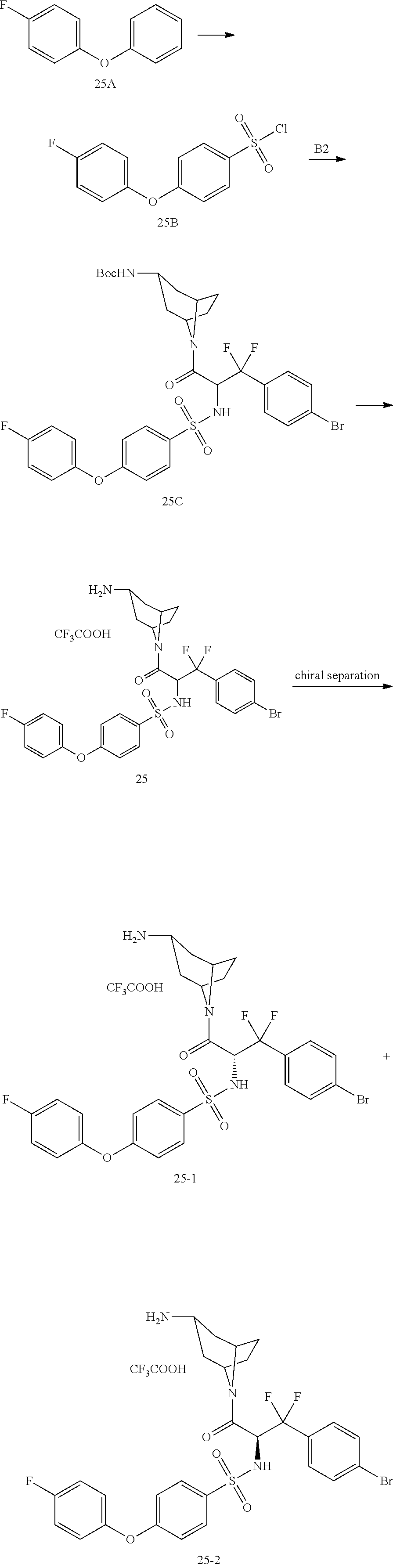

To ice-cooled Compound 25A (0.5 g, 2.7 mmol) was dropped chlorosulfonic acid (0.6 g, 5.3 mmol) and stirred at 25° C. for 4 hours. The reaction mixture was poured into ice-water (20 mL) and extracted with ethyl acetate (20 mL×2). The combined organic extracts was washed with water (10 mL) and brine (10 mL), dried over anhydrous sodium sulfate, filtered, concentrated, and purified by silica gel column chromatography to give Compound 25B. LC-MS (ESI) m/z: 282 [M−Cl+CH₃NH₂]⁺; ¹H-NMR (CDCl₃, 400 MHz): δ (ppm) 7.05-7.17 (m, 6H), 7.97-7.99 (m, 2H).

Compounds 25C and 25 were synthesized by employing the procedures described for Compounds 2D and 1 using Compounds 25B and 25C in lieu of Compounds 2C and 1A.

Compound 25C: LC-MS (ESI) m/z: 738 [M+H]⁺.

Compound 25 was separated with chiral HPLC to furnish Compound 25-1 and Compound 25-2.

Compound 25-1: LC-MS (ESI) m/z: 638 [M+H]⁺; ¹H-NMR (CD₃OD, 400 MHz): δ (ppm) 1.58-2.12 (m, 8H), 3.63-3.73 (m, 1H), 4.59-4.88 (m, 3H), 6.91-6.95 (m, 2H), 7.10-7.21 (m, 4H), 7.29-7.31 (m, 1H), 7.45-7.49 (m, 2H), 7.59-7.64 (m, 3H). Chiral separation condition: MeOH contained 0.1% DEA; OJ-H (4.6×250 mm, 5 μm); retention time: 3.88 minute.

Compound 25-2: LC-MS (ESI) m/z: 638 [M+H]⁺; ¹H-NMR (CD₃OD, 400 MHz): δ (ppm) 1.47-2.04 (m, 8H), 3.54-3.61 (m, 1H), 4.49-4.76 (m, 3H), 6.81-6.85 (m, 2H), 7.06-7.10 (m, 4H), 7.18-7.20 (m, 1H), 7.32-7.38 (m, 2H), 7.47-7.54 (m, 3H). Chiral separation condition: MeOH contained 0.1% DEA; OJ-H (4.6×250 mm, 5 μm); retention time: 2.67 minute.

### Example 26

### Synthesis of N-((2R)-3-(3-amino-8-azabicyclo[3.2.1]octan-8-yl)-1-(4-bromophenyl)-1,1-difluoro-3-oxopropan-2-yl)-4-(2-cyclohexylethoxy)benzenesulfonamide trifluoroacetate (Compound 26-1), and N-((2S)-3-(3-amino-8-azabicyclo[3.2.1]octan-8-yl)-1-(4-bromophenyl)-1,1-difluoro-3-oxopropan-2-yl)-4-(2-cyclohexylethoxy)benzenesulfonamide trifluoroacetate (Compound 26-2)

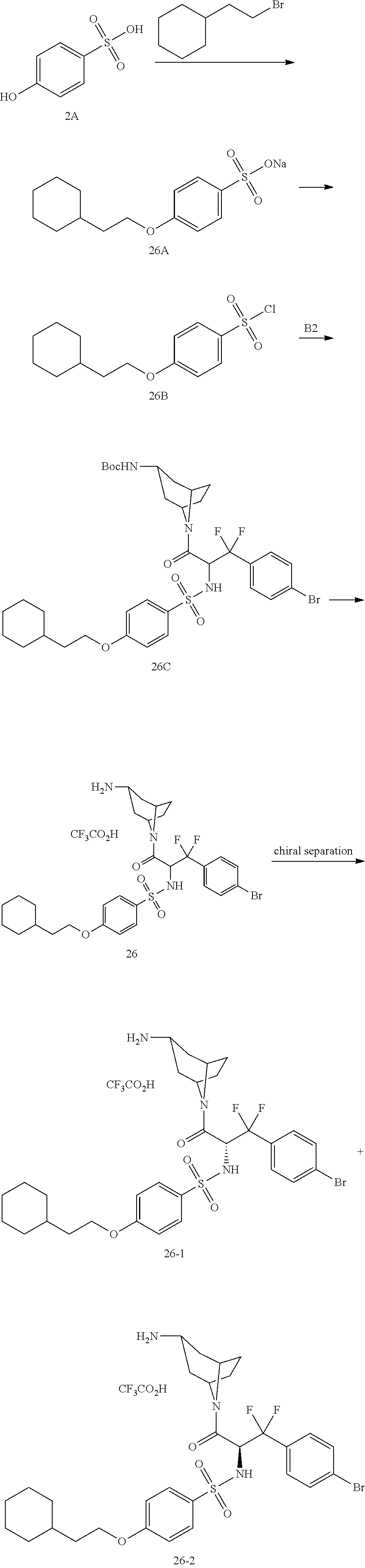

Compounds 26A, 26B, 26C, and 26 were synthesized by employing the procedures described for Compound 2B, Compound A1, Compounds 2D, and 1 using (2-bromoethyl)cyclohexane, Compounds 26A, 26B, and 26C in lieu of 3-(bromomethyl)pentane, Compound A1-3, Compounds 2C, and 1A.

Compound 26A: LC-MS (ESI) m/z: 283 [M−Na]⁺.

Compound 26B: LC-MS (ESI) m/z: non-ionizable compound under routine conditions used; ¹H-NMR (CDCl₃, 400 MHz): δ (ppm) 0.82-0.94 (m, 3H), 1.03-1.13 (m, 4H), 1.45-1.52 (m, 1H), 1.66-1.73 (m, 5H), 4.10 (t, J=5.4 Hz, 2H), 7.02 (d, J=8.8 Hz, 2H), 7.96 (d, J=8.8 Hz, 2H).

Compound 26C: LC-MS (ESI) m/z: 776 [M+Na]⁺.

Compound 26 was separated with chiral HPLC to give Compound 26-1 and Compound 26-2.

Compound 26-1: LC-MS (ESI) m/z: 654 [M+H]⁺; ¹H-NMR (CD₃OD, 400 MHz): δ (ppm) 0.99-1.08 (m, 2H), 1.21-1.37 (m, 2H), 1.53-2.16 (m, 16H), 3.60-3.74 (m, 1H), 4.08-4.14 (m, 2H), 4.57-4.82 (m, 3H), 6.87-6.95 (m, 2H), 7.25-7.28 (m, 2H), 7.41-7.44 (m, 2H), 7.51-7.57 (m, 1H), 7.58-7.60 (m, 2H). Chiral separation condition: MeOH contained 0.1% DEA; OJ-H (4.6×250 mm, 5 μm); retention time: 5.10 minute.

Compound 26-2: LC-MS (ESI) m/z: 654 [M+H]⁺; ¹H-NMR (CD₃OD, 400 MHz): δ (ppm) 0.97-1.08 (m, 2H), 1.20-1.34 (m, 2H), 1.50-2.18 (m, 16H), 3.62-3.76 (m, 1H), 4.07-4.14 (m, 2H), 4.57-4.82 (m, 3H), 6.87-6.95 (m, 2H), 7.25-7.28 (m, 2H), 7.41-7.44 (m, 2H), 7.51-7.57 (m, 1H), 7.58-7.60 (m, 2H). Chiral separation condition: MeOH contained 0.1% DEA; OJ-H (4.6×250 mm, 5 μm); retention time: 3.09 minute.

### Example 27

### Synthesis of (R)—N-(3-(4-aminopiperidin-1-yl)-1-(4-bromophenyl)-1,1-difluoro-3-oxopropan-2-yl)-6-isopropoxynaphthalene-2-sulfonamide trifluoroacetate (Compound 27-1), and (S)—N-(3-(4-aminopiperidin-1-yl)-1-(4-bromophenyl)-1,1-difluoro-3-oxopropan-2-yl)-6-isopropoxynaphthalene-2-sulfonamide trifluoroacetate (Compound 27-2)

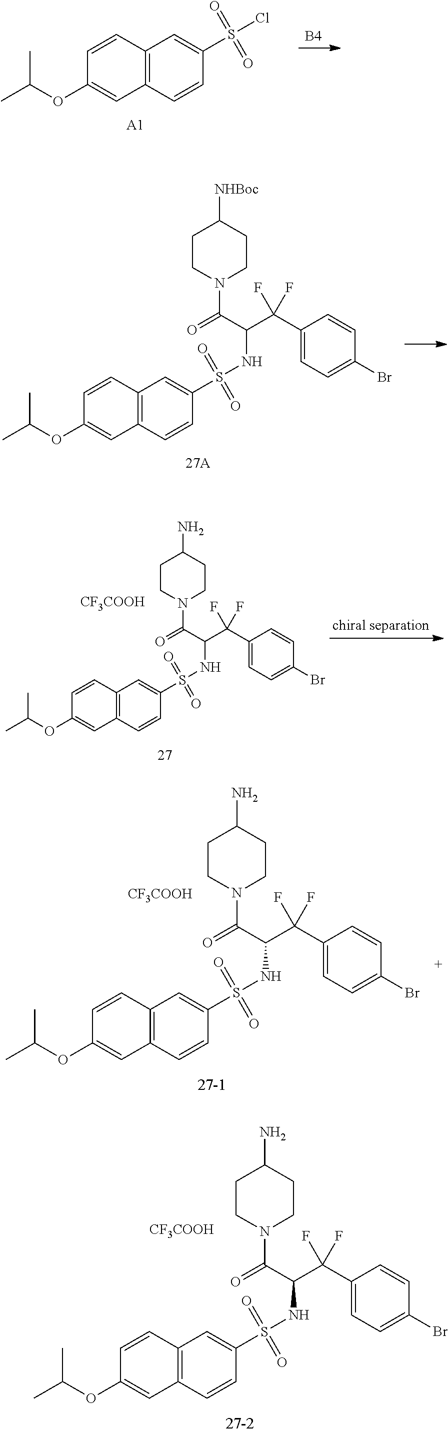

Compounds 27A and 27 were synthesized by employing the procedures described for Compounds 2D and 1 using Compounds B4, A1, and Compound 27A in lieu of Compound B2, Compounds 2C, and 1A.

Compound 27A: LC-MS (ESI) m/z: 654 [M−55]⁺, 732 [M+Na]⁺.

Compound 27: LC-MS (ESI) m/z: 610 [M+H]⁺, which was separated with chiral HPLC (n-hexane/EtOH contained 0.1% DEA, 50/50; AD-H, 250×4.6 mm, 5 m) to furnish Compound 27-1 and Compound 27-2.

Compound 27-1: LC-MS (ESI) (m/z) 610 [M+H]⁺; ¹H-NMR (CD₃OD, 400 MHz): δ (ppm) 1.26-1.36 (m, 1H), 1.40-1.48 (m, 6H), 1.68-1.85 (m, 1H), 2.03-2.16 (m, 2H), 2.73-2.97 (m, 1H), 3.19 (t, J=12.1 Hz, 1H), 3.38-3.44 (m, 1H), 4.15 (d, J=13.5 Hz, 1H), 4.29-4.57 (m, 1H), 4.79-4.86 (m, 1H), 4.94-5.07 (m, 1H), 7.22-7.26 (m, 3H), 7.32 (s, 1H), 7.36-7.38 (m, 1H), 7.42-7.55 (m, 2H), 7.70-7.89 (m, 2H), 8.11 (d, J=34.4, 1H). Chiral separation condition: n-hexane/EtOH contained 0.1% DEA, 50/50; AD-H (250×4.6 mm, 5 μm); retention time: 8.90 minute.

Compound 27-2: LC-MS (ESI) (m/z) 610 [M+H]⁺; ¹H-NMR (CD₃OD, 400 MHz): δ (ppm) 1.26-1.36 (m, 1H), 1.40-1.48 (m, 6H), 1.68-1.84 (m, 1H), 2.03-2.16 (m, 2H), 2.74-2.98 (m, 1H), 3.19 (t, J=12.2 Hz, 1H), 3.41 (t, J=11.6 Hz, 1H), 4.15 (d, J=13.9 Hz, 1H), 4.29-4.57 (m, 1H), 4.80-4.85 (m, 1H), 4.95-5.07 (m, 1H), 7.22-7.26 (m, 3H), 7.33-7.55 (m, 4H), 7.70-7.89 (m, 2H), 8.11 (d, J=34.2, 1H). Chiral separation condition: n-hexane/EtOH contained 0.1% DEA, 50/50; AD-H (250×4.6 mm, 5 μm); retention time: 6.32 minute.

### Example 28

### Synthesis of N-((2R)-3-(3-amino-8-azabicyclo[3.2.1]octan-8-yl)-1-(4-chlorophenyl)-1,1-difluoro-3-oxopropan-2-yl)-6-(cyclopentyloxy)naphthalene-2-sulfonamide trifluoroacetate (Compound 28-1), and N-((2S)-3-(3-amino-8-azabicyclo[3.2.1]octan-8-yl)-1-(4-chlorophenyl)-1,1-difluoro-3-oxopropan-2-yl)-6-(cyclopentyloxy)naphthalene-2-sulfonamide trifluoroacetate (Compound 28-2)

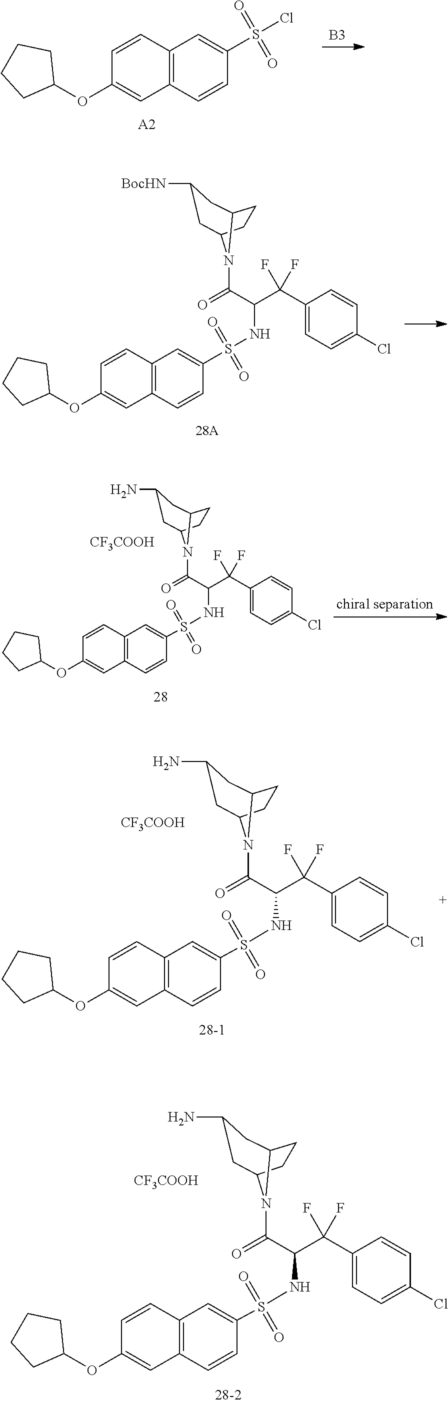

Compounds 28A and 28 were synthesized by employing the procedures described for Compounds 2D and 1 using Compounds B3, A2, and Compound 28A in lieu of Compound B2, Compounds 2C, and 1A.

Compound 28A: LC-MS (ESI) m/z: 740 [M+Na]⁺.

Compound 28, which was separated with chiral HPLC (MeOH contained 0.5% DEA; (R,R)-Whelk-O1, 250×4.6 mm, 5 m) to give Compound 28-1 and Compound 28-2.

Compound 28-1: LC-MS (ESI) m/z: 618 [M+H]⁺; ¹H-NMR (CD₃OD, 400 MHz): δ (ppm) 0.73-2.21 (m, 16H), 3.50-3.73 (m, 1H), 4.36-4.56 (m, 1H), 4.66-4.86 (m, 2H), 5.01 (d, J=6.4 Hz, 1H), 7.01 (d, J=8.8 Hz, 1H), 7.22-7.36 (m, 4H), 7.45-7.59 (m, 2H), 7.70-7.87 (m, 2H), 8.07-8.17 (m, 1H); Chiral separation condition: MeOH contained 0.5% DEA; (R,R)-Whelk-O1 (250×4.6 mm, 5 μm); retention time: 3.76 minute.

Compound 28-2: LC-MS (ESI) m/z: 618 [M+H]⁺; ¹H-NMR (CD₃OD, 400 MHz): δ (ppm) 0.78-2.20 (m, 16H), 3.48-3.72 (m, 1H), 4.35-4.56 (m, 1H), 4.65-4.81 (m, 2H), 5.01 (s, 1H), 7.00 (d, J=8.8 Hz, 1H), 7.21-7.36 (m, 4H), 7.44-7.59 (m, 2H), 7.70-7.87 (m, 2H), 8.06-8.17 (m, 1H); Chiral separation condition: MeOH contained 0.5% DEA; (R,R)-Whelk-O1 (250×4.6 mm, 5 μm); retention time: 4.55 minute.

### Example 29

### Synthesis of N-((2R)-3-(3-amino-8-azabicyclo[3.2.1]octan-8-yl)-1-(4-chlorophenyl)-1,1-difluoro-3-oxopropan-2-yl)-4-(cyclohexylmethoxy)benzenesulfonamide trifluoroacetate (Compound 29-1), and N-((2S)-3-(3-amino-8-azabicyclo[3.2.1]octan-8-yl)-1-(4-chlorophenyl)-1,1-difluoro-3-oxopropan-2-yl)-4-(cyclohexylmethoxy) benzenesulfonamide trifluoroacetate (Compound 29-2)

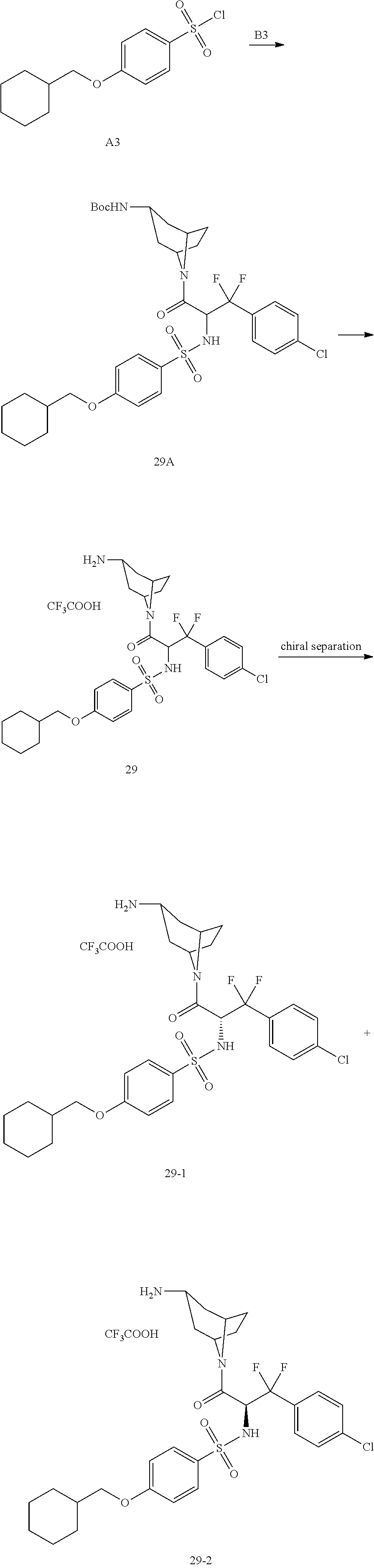

Compounds 29A and 29 were synthesized by employing the procedures described for Compounds 2D and 1 using Compounds B3, A3, and Compound 29A in lieu of Compound B2, Compounds 2C, and 1A.

Compound 29A: LC-MS (ESI) m/z: 718 [M+Na]⁺.

Compound 29, which was separated with chiral HPLC (MeOH contained 0.5% DEA; (R,R)-Whelk-O1, 250×4.6 mm, 5 μm) to give Compound 29-1 and Compound 29-.

Compound 29-1: LC-MS (ESI) m/z: 596 [M+H]⁺; ¹H-NMR (CD₃OD, 400 MHz): δ (ppm) 1.06-2.17 (m, 20H), 3.59-3.75 (m, 1H), 3.83-3.87 (m, 2H), 4.56-4.65 (m, 2H), 4.68-4.90 (m, 2H), 6.86-6.93 (m, 2H), 7.30 (dd, J₁=18.4 Hz, J₂=8.8 Hz, 1H), 7.41 (d, J=8.8 Hz, 1H), 7.47-7.51 (m, 2H), 7.58 (d, J=8.4 Hz, 1H). Chiral separation condition: MeOH contained 0.5% DEA; (R,R)-Whelk-O1 (250×4.6 mm, 5 μm); retention time: 4.86 minute.

Compound 29-2: LC-MS (ESI) m/z: 596 [M+H]⁺; ¹H-NMR (CD₃OD, 400 MHz): δ (ppm) 1.04-2.16 (m, 20H), 3.60-3.76 (m, 1H), 3.83-3.88 (m, 2H), 4.56-4.86 (m, 4H), 6.87-6.94 (m, 2H), 7.30 (dd, J₁=18.4 Hz, J₂=8.8 Hz, 1H), 7.41 (d, J=8.4 Hz, 1H), 7.47-7.51 (m, 2H), 7.58 (d, J=8.8 Hz, 1H); Chiral separation condition: MeOH contained 0.5% DEA; (R,R)-Whelk-O1 (250×4.6 mm, 5 μm); retention time: 5.62 minute.

### Example 30

### Synthesis of (R)—N-(3-(4-aminopiperidin-1-yl)-1-(4-chlorophenyl)-1,1-difluoro-3-oxopropan-2-yl)-4-(cyclohexylmethoxy)benzenesulfonamide trifluoroacetate (Compound 30-1), and (S)—N-(3-(4-aminopiperidin-1-yl)-1-(4-chlorophenyl)-1,1-difluoro-3-oxopropan-2-yl)-4-(cyclohexylmethoxy)benzenesulfonamide trifluoroacetate (Compound 30-2)

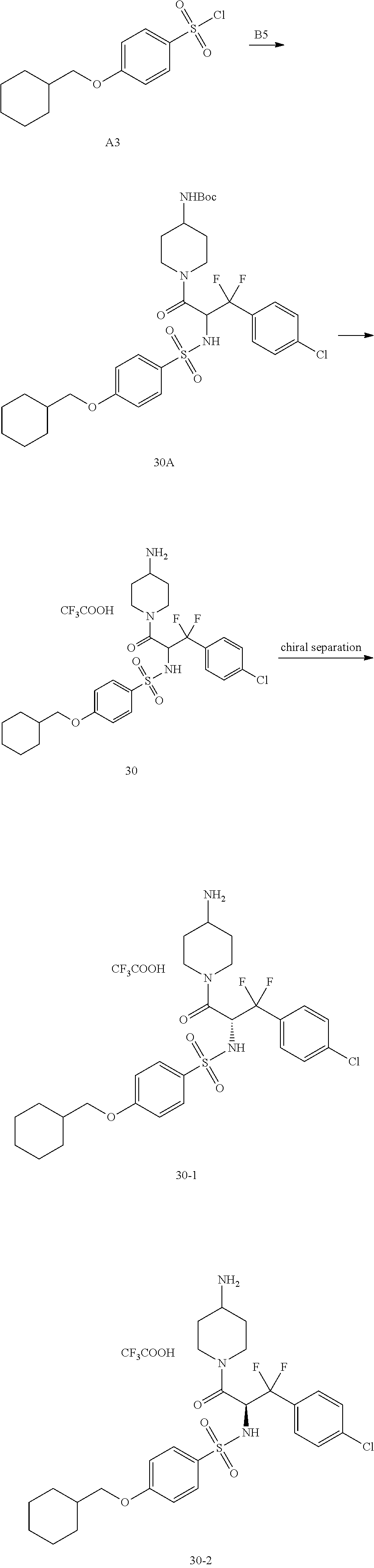

Compounds 30A and 30 were synthesized by employing the procedures described for Compounds 2D and 1 using Compounds B5, A3, and Compound 30A in lieu of Compound B2, Compounds 2C, and 1A.

Compound 30A: LC-MS (ESI) m/z: 692 [M+Na]⁺.

Compound 30, which was separated with chiral HPLC (MeOH contained 0.5% DEA; OJ, 250×4.6 mm, 5 μm) to give Compound 30-1 and Compound 30-2.

Compound 30-1: LC-MS (ESI) m/z: 570 [M+H]⁺; ¹H-NMR (CD₃OD, 400 MHz): δ (ppm) 1.06-2.15 (m, 16H), 2.42-2.79 (m, 1H), 3.06-3.21 (m, 1H), 3.35-3.44 (m, 1H), 3.86 (d, J=9.2 Hz, 2H), 4.15-4.56 (m, 2H), 6.88-6.94 (m, 2H), 7.29-7.56 (m, 6H). Chiral separation condition: MeOH contained 0.5% DEA; OJ (250×4.6 mm, 5 μm); retention time: 3.62 minute.

Compound 30-2: LC-MS (ESI) m/z: 570 [M+H]⁺; ¹H-NMR (CD₃OD, 400 MHz): δ (ppm) 1.05-2.14 (m, 16H), 2.43-2.80 (m, 1H), 3.07-3.47 (m, 2H), 3.86 (d, J=9.2 Hz, 2H), 4.16-4.58 (m, 2H), 6.88-6.94 (m, 2H), 7.28-7.56 (m, 6H); Chiral separation condition: MeOH contained 0.5% DEA; OJ (250×4.6 mm, 5 μm); retention time: 2.1 minute.

### Example 31

### Synthesis of N-((2R)-1-([1,1′-biphenyl]-4-yl)-3-(3-amino-8-azabicyclo[3.2.1]octan-8-yl)-1,1-difluoro-3-oxopropan-2-yl)-6-isopropoxynaphthalene-2-sulfonamide trifluoroacetate (Compound 31-1), and N-((2S)-1-([1,1′-biphenyl]-4-yl)-3-(3-amino-8-azabicyclo[3.2.1]octan-8-yl)-1,1-difluoro-3-oxopropan-2-yl)-6-isopropoxynaphthalene-2-sulfonamide trifluoroacetate (Compound 31-2)

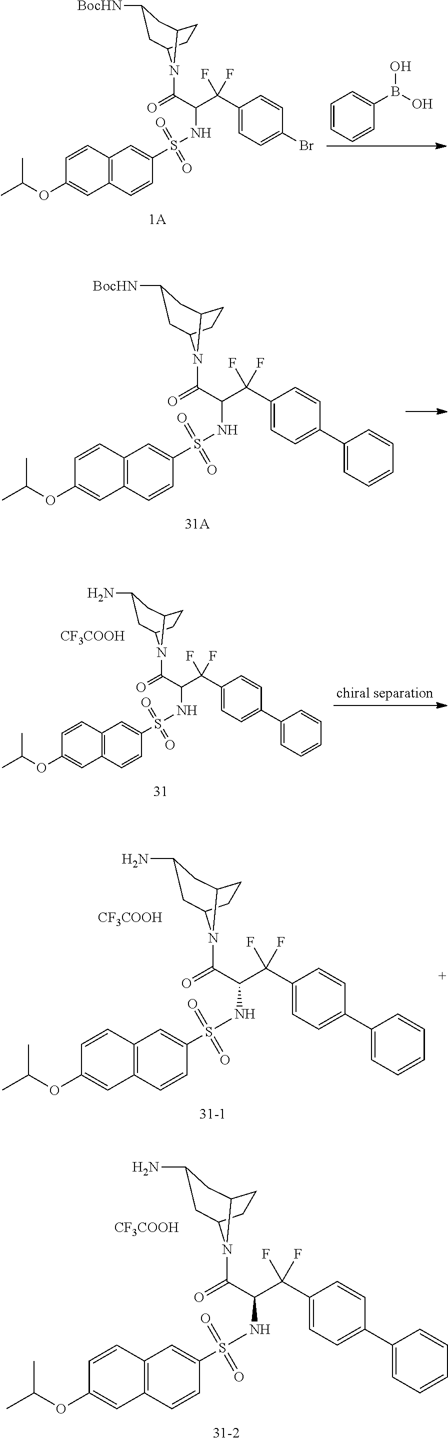

Compounds 31A and 31 were synthesized by employing the procedures described for Compounds 14C and 1 using phenylboronic acid, Compounds 1A, and 31A in lieu of Compounds 14B, 14A, and 1A.

Compound 31A: LC-MS (ESI) m/z: 756 [M+Na]⁺; ¹H-NMR (CDCl₃, 400 MHz): δ (ppm) 0.41-0.47 (m, 1H), 0.77-0.90 (m, 2H), 1.38-1.41 (m, 4H), 1.46 (s, 6H), 1.60 (s, 9H), 1.70-1.83 (m, 2H), 3.47 (d, J=8.8 Hz, 1H), 3.76-3.83 (m, 1H), 4.47-4.72 (m, 3H), 6.10 (d, J=8.8 Hz, 1H), 7.07-7.10 (m, 1H), 7.18-7.26 (m, 1H), 7.37-7.49 (m, 5H), 7.53-7.66 (m, 4H), 7.71-7.81 (m, 3H), 8.18-8.24 (m, 1H).

Compound 31, which was separated with chiral HPLC to give Compound 31-1 and Compound 31-2.

Compound 31-1: LC-MS (ESI) m/z: 634 [M+H]⁺; ¹H-NMR (CD₃OD, 400 MHz): δ (ppm) 1.32-1.43 (m, 7H), 1.55-1.77 (m, 2H), 1.80-2.19 (m, 5H), 3.48-3.66 (m, 1H), 4.42-4.74 (m, 3H), 4.96-5.10 (m, 1H), 7.00-7.24 (m, 3H), 7.30-7.45 (m, 6H), 7.51-7.85 (m, 5H), 8.03-8.17 (m, 1H). Chiral separation condition: n-hexane/EtOH contained 0.1% DEA, 50/50; (S, S)-Whelk-O1 (4.6×250 mm, 5 μm); retention time: 12.62 minute.

Compound 31-2: LC-MS (ESI) m/z: 634 [M+H]⁺; ¹H-NMR (CD₃OD, 400 MHz): δ (ppm) 1.34-1.43 (m, 6H), 1.51-2.20 (m, 8H), 3.55-3.74 (m, 1H), 4.43-4.74 (m, 4H), 7.00-7.17 (m, 2H), 7.30-7.50 (m, 6H), 7.55-7.84 (m, 6H), 8.10-8.17 (m, 1H). Chiral separation condition: n-hexane/EtOH contained 0.1% DEA, 50/50; (S, S)-Whelk-O1 (4.6×250 mm, 5 μm); retention time: 10.47 minute.

### Example 32

### Synthesis of (R)—N-(3-(4-aminopiperidin-1-yl)-1-(4-bromophenyl)-1,1-difluoro-3-oxopropan-2-yl)-6-(cyclopentyloxy)naphthalene-2-sulfonamide trifluoroacetate (Compound 32-1), and (S)—N-(3-(4-aminopiperidin-1-yl)-1-(4-bromophenyl)-1,1-difluoro-3-oxopropan-2-yl)-6-(cyclopentyloxy)naphthalene-2-sulfonamide trifluoroacetate (Compound 32-2)

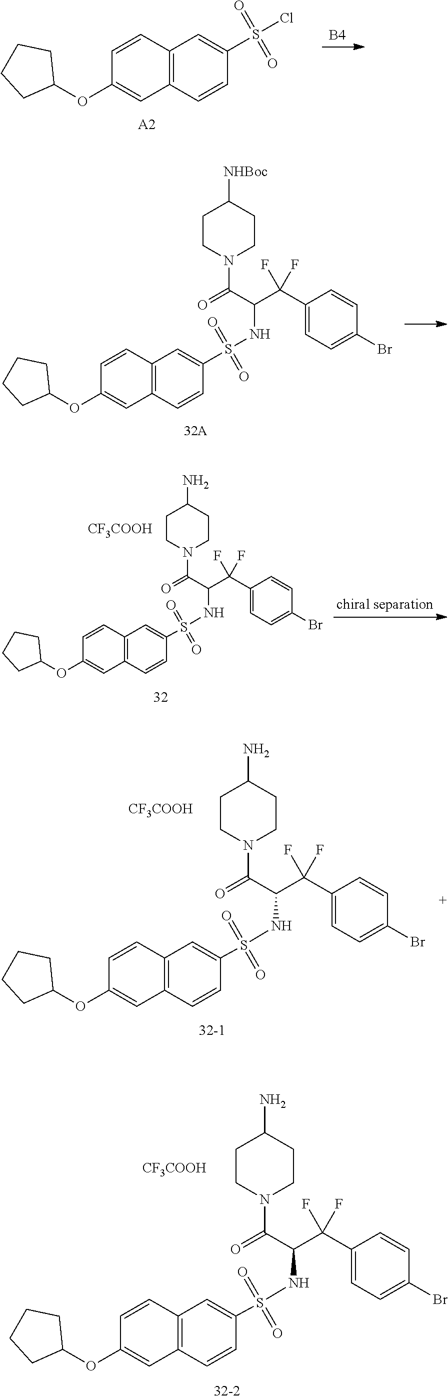

Compounds 32A and 32 were synthesized by employing the procedures described for Compounds 2D and 1 using Compounds B4, A2, and Compound 32A in lieu of Compound B2, Compounds 2C, and 1A.

Compound 32A: LC-MS (ESI) m/z: 680 [M−55]⁺, 758 [M+Na]⁺.

Compound 32: LC-MS (ESI) m/z: 636 [M+H]⁺. It was separated with chiral HPLC (n-hexane contained/EtOH contained 0.1% DEA, 70/30; AD-H, 250×4.6 mm, 5 μm) to furnish Compound 32-1 and Compound 32-2.

Compound 32-1: LC-MS (m/z): 636 [M+H]⁺; ¹H-NMR (CD₃OD, 400 MHz): δ (ppm) 1.29-2.16 (m, 13H), 2.74-3.00 (m, 1H), 3.16-3.19 (m, 1H), 4.16 (d, J=5.6 Hz, 1H), 4.30-4.60 (m, 1H), 4.92-5.07 (m, 2H), 7.21-7.55 (m, 7H), 7.70-7.88 (m, 2H), 8.11 (d, J=35.2 Hz, 1H). Chiral separation condition: n-hexane/EtOH contained 0.1% DEA, 70/30; AD-H (250×4.6 mm, 5 μm); retention time: 19.17 minute.

Compound 32-2: LC-MS (m/z): 636 [M+H]⁺; ¹H-NMR (CD₃OD, 400 MHz): δ (ppm) 1.29-2.16 (m, 13H), 2.74-2.99 (m, 1H), 3.17-3.21 (m, 1H), 4.15 (d, J=5.6 Hz, 1H), 4.29-4.61 (m, 1H), 4.92-5.07 (m, 2H), 7.21-7.54 (m, 7H), 7.70-7.88 (m, 2H), 8.11 (d, J=35.2 Hz, 1H). Chiral separation condition: n-hexane/EtOH contained 0.1% DEA, 70/30; AD-H (250×4.6 mm, 5 μm); retention time: 12.85 minute.

### Example 33

### Synthesis of N-((2R)-3-(3-amino-8-azabicyclo[3.2.1]octan-8-yl)-1-(4-bromophenyl)-1,1-difluoro-3-oxopropan-2-yl)-6-(cyclohexyloxy)naphthalene-2-sulfonamide trifluoroacetate (Compound 33-1), and N-((2S)-3-(3-amino-8-azabicyclo[3.2.1]octan-8-yl)-1-(4-bromophenyl)-1,1-difluoro-3-oxopropan-2-yl)-6-(cyclohexyloxy)naphthalene-2-sulfonamide trifluoroacetate (Compound 33-2)

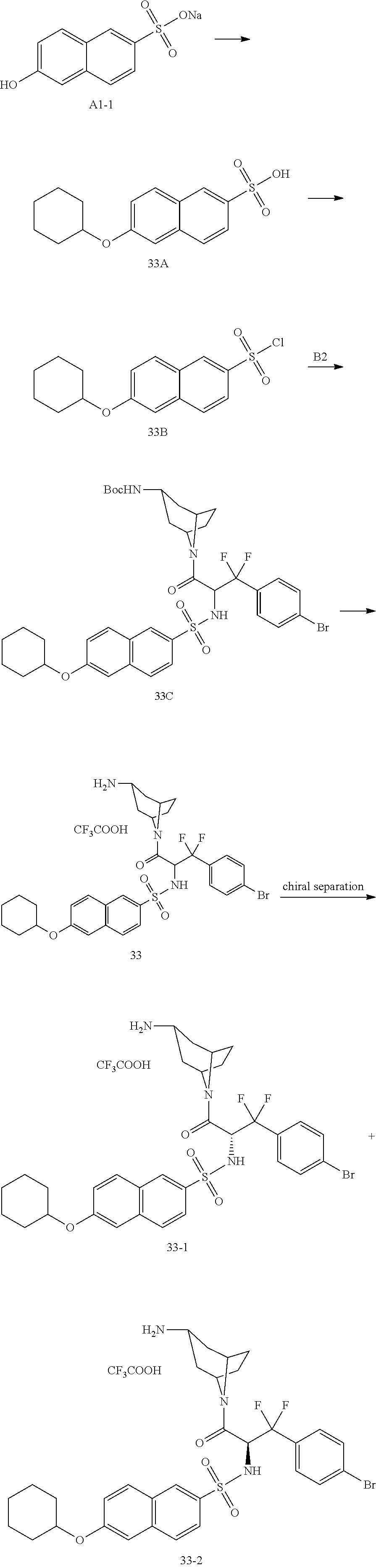

To a solution of Compound A1-1 (1.23 g, 5 mmol) and potassium carbonate (2.76 g, 20 mmol) in N,N-dimethylacetamide (15 mL) was added bromocyclohexane (5.2 g, 30 mmol) and heated at 100° C. for 12 hours. The reaction mixture was purified with preparative HPLC to furnish a crude Compound 33A. LC-MS (ESI) m/z: 305 [M−H]⁺.

Compounds 33B, 33C, and 33 were synthesized by employing the procedures described for Compound A1, Compounds 2D, and 1 using Compounds 33A, 33B, and 33C in lieu of Compound A1-3, Compounds 2C, and 1A.

Compound 33B: ¹H-NMR (CDCl₃, 400 MHz): δ (ppm) 0.82-1.50 (m, 7H), 1.84-2.08 (m, 3H), 4.46-4.51 (m, 1H), 7.21 (s, 1H), 7.29-7.32 (m, 1H), 7.85-8.05 (m, 3H), 8.41-8.52 (m, 1H).

Compound 33C: LC-MS (ESI) m/z: 798 [M+Na]⁺.

Compound 33, which was separated with chiral HPLC (MeOH contained 0.1% DEA; IC, 250×4.6 mm, 5 m) to give Compound 33-1 and Compound 33-2.

Compound 33-1: LC-MS (ESI) m/z: 676 [M+H]⁺; ¹H-NMR (CD₃OD, 400 MHz): δ (ppm) 0.80-2.20 (m, 19H), 3.49-3.75 (m, 1H), 4.36-4.60 (m, 2H), 4.66-4.72 (m, 1H), 7.18 (s, 2H), 7.25-7.29 (m, 1H), 7.34 (t, J=2.8 Hz, 1H), 7.41-7.58 (m, 3H), 7.70-7.88 (m, 2H), 8.12 (d, 1H). Chiral separation condition: n-hexane/EtOH contained 0.1% DEA, 60/40; AD-H (250×4.6 mm, 5 μm); retention time: 19.79 minute.

Compound 33-2: LC-MS (ESI) m/z: 676 [M+H]⁺; ¹H-NMR (CD₃OD, 400 MHz): δ (ppm) 0.80-2.20 (m, 19H), 3.49-3.75 (m, 1H), 4.36-4.60 (m, 2H), 4.66-4.72 (m, 1H), 7.18 (s, 2H), 7.25-7.29 (m, 1H), 7.34 (t, J=2.8 Hz, 1H), 7.41-7.58 (m, 3H), 7.70-7.88 (m, 2H), 8.12 (d, 1H). Chiral separation condition: n-hexane/EtOH contained 0.1% DEA, 60/40; AD-H (250×4.6 mm, 5 μm); retention time: 10.29 min.

### Example 34

### Synthesis of N-((2R)-3-(3-amino-8-azabicyclo[3.2.1]octan-8-yl)-1-(4-bromophenyl)-1,1-difluoro-3-oxopropan-2-yl)-6-(cyclopentyloxy)-N-methylnaphthalene-2-sulfonamide trifluoroacetate (Compound 34-1), and N-((2S)-3-(3-amino-8-azabicyclo[3.2.1]octan-8-yl)-1-(4-bromophenyl)-1,1-difluoro-3-oxopropan-2-yl)-6-(cyclopentyloxy)-N-methylnaphthalene-2-sulfonamide trifluoroacetate (Compound 34-2)

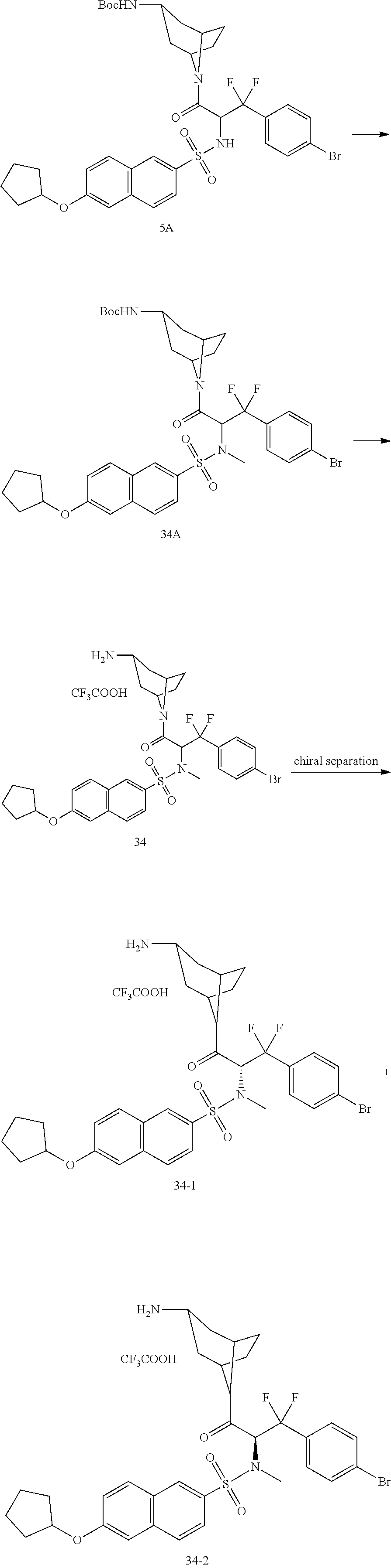

To a solution of Compound 5A (120 mg, 0.16 mmol) and K₂CO₃ (44 mg, 0.31 mmol) in DMSO (2 mL) was added iodomethane (34 mg, 0.24 mmol) at 0° C. The reaction mixture was stirred at 0° C. for 3 hours, quenched with saturated ammonium chloride solution (3 mL), and extracted with ethyl acetate (5 mL×3). The combined organic extracts was washed with water (5 mL) and brine (5 mL), dried over anhydrous sodium sulfate, filtered, and concentrated. The crude product was purified with preparative TLC) to yield Compound 34A. LC-MS (ESI) m/z: 720 [M−55]⁺, 798 [M+Na]⁺.

Compound 34 was synthesized by employing the procedure described for Compound 1 using Compound 34A in lieu of Compound 1A, which was separated with chiral HPLC (n-hexane/EtOH contained 0.1% DEA, 50/50; AY-H, 250×4.6 mm, 5 m) to give Compound 34-1 and Compound 34-2.

Compound 34-1: LC-MS (ESI) m/z: 676 [M+H]⁺; ¹H-NMR (CD₃OD, 400 MHz): δ (ppm) 1.63-2.11 (m, 16H), 3.05 (s, 1H), 3.23 (s, 2H), 3.66-3.70 (m, 1H), 4.58-4.73 (m, 2H), 5.01-5.04 (m, 1H), 5.53-5.60 (m, 1H), 7.25-7.28 (m, 1H), 7.32-7.55 (m, 6H), 7.77-7.94 (m, 2H), 8.11-8.25 (m, 1H). Chiral separation condition: n-hexane/EtOH contained 0.1% DEA, 50/50; AY-H, 250×4.6 mm, 5 μm); retention time: 15.28 minute.

Compound 34-2: LC-MS (ESI) m/z: 676 [M+H]⁺; ¹H-NMR (CD₃OD, 400 MHz): δ (ppm) 1.29-2.19 (m, 16H), 3.05 (s, 1H), 3.23 (s, 2H), 3.66-3.70 (m, 1H), 4.55-4.66 (m, 2H), 5.01-5.04 (m, 1H), 5.53-5.60 (m, 1H), 7.24-7.28 (m, 1H), 7.31-7.55 (m, 6H), 7.77-7.94 (m, 2H), 8.11-8.24 (m, 1H). Chiral separation condition: n-hexane/EtOH contained 0.1% DEA, 50/50; AY-H, 250×4.6 mm, 5 μm); retention time: 6.96 minute.

### Example 35

### Synthesis of N-((2R)-1-([1,1′-biphenyl]-4-yl)-3-(3-amino-8-azabicyclo[3.2.1]octan-8-yl)-1,1-difluoro-3-oxopropan-2-yl)-4-(cyclohexylmethoxy)benzenesulfonamide trifluoroacetate (Compound 35-1), and N-((2S)-1-([1,1′-biphenyl]-4-yl)-3-(3-amino-8-azabicyclo[3.2.1]octan-8-yl)-1,1-difluoro-3-oxopropan-2-yl)-4-(cyclohexylmethoxy) benzenesulfonamide trifluoroacetate (Compound 35-2)

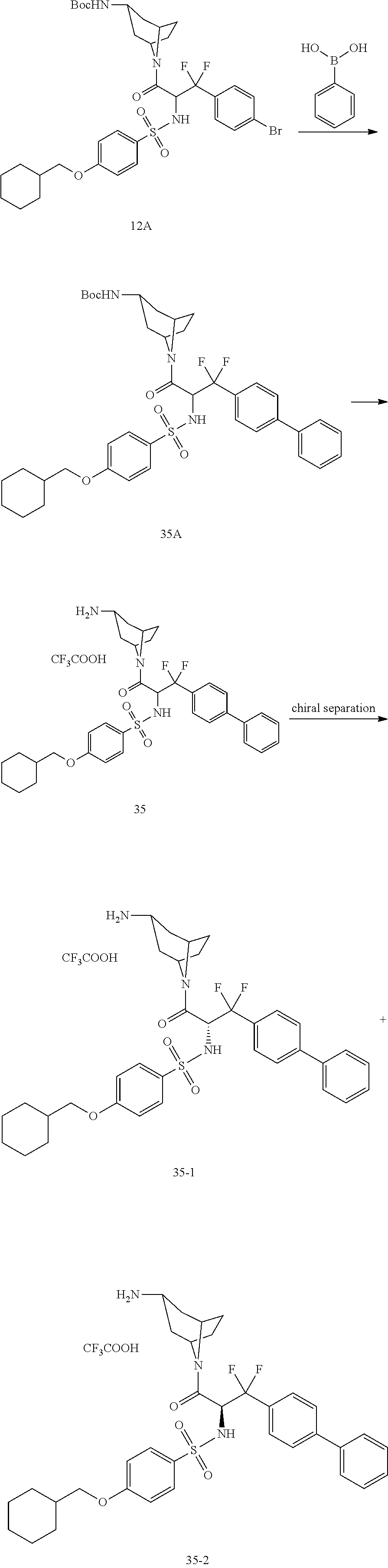

Compounds 35A and 35 were synthesized by employing the procedures described for Compounds 14C and 1 using phenylboronic acid, Compounds 12A, and 35A in lieu of Compounds 14B, 14A, and 1A.

Compound 35A: LC-MS (ESI) m/z: 738 [M+H]⁺.

Compound 35 was separated with chiral HPLC to furnish Compound 35-1 and Compound 35-2.

Compound 35-1: LC-MS (ESI) m/z: 638 [M+H]⁺; ¹H-NMR (CD₃OD, 400 MHz): δ (ppm) 0.92-1.06 (m, 2H), 1.21-1.35 (m, 3H), 1.64-2.17 (m, 14H), 3.49-3.72 (m, 3H), 4.59-4.85 (m, 3H), 6.77-6.85 (m, 2H), 7.41-7.43 (m, 2H), 7.48-7.56 (m, 6H), 7.63-7.69 (m, 3H). Chiral separation condition: MeOH contained 0.5% DEA; RegisCell (4.6×250 mm, 5 μm); retention time: 7.64 minute.

Compound 35-2: LC-MS (ESI) m/z: 638 [M+H]⁺; ¹H-NMR (CD₃OD, 400 MHz): δ (ppm) 0.93-1.06 (m, 2H), 1.20-1.36 (m, 3H), 1.64-2.14 (m, 14H), 3.51-3.72 (m, 3H), 4.58-4.84 (m, 3H), 6.77-6.85 (m, 2H), 7.41-7.43 (m, 2H), 7.48-7.56 (m, 6H), 7.63-7.69 (m, 3H). Chiral separation condition: MeOH contained 0.5% DEA; RegisCell (4.6×250 mm, 5 μm); retention time: 5.89 minute.

### Example 36

### Synthesis of N-((2R)-1-([1,1′-biphenyl]-4-yl)-3-(3-amino-8-azabicyclo[3.2.1]octan-8-yl)-1,1-difluoro-3-oxopropan-2-yl)-6-(cyclopentyloxy)naphthalene-2-sulfonamide trifluoroacetate (Compound 36-1), and N-((2S)-1-([1,1′-biphenyl]-4-yl)-3-(3-amino-8-azabicyclo[3.2.1]octan-8-yl)-1,1-difluoro-3-oxopropan-2-yl)-6-(cyclopentyloxy)naphthalene-2-sulfonamide trifluoroacetate (Compound 36-2)

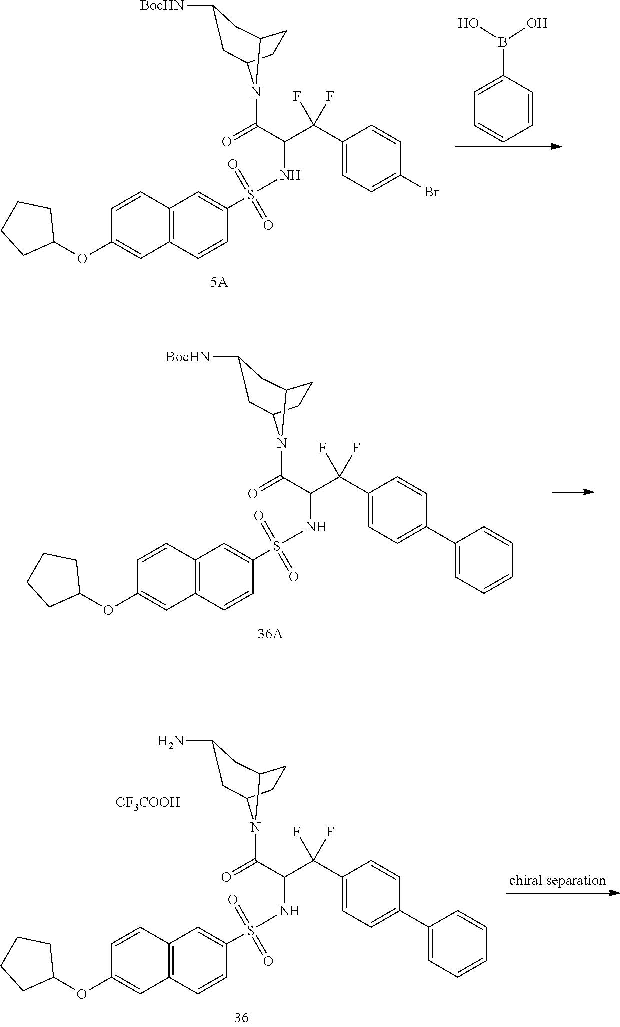 [SMILES: CC(C)(C)OC(=O)NC1CC2CCC(C1)N2C(=O)C(NS(=O)(=O)c1ccc2cc(OC3CCCC3)ccc2c1)C(F)(F)c1ccc(Br)cc1 ; OB(O)c1ccccc1 ; CC(C)(C)OC(=O)NC1CC2CCC(C1)N2C(=O)C(NS(=O)(=O)c1ccc2cc(OC3CCCC3)ccc2c1)C(F)(F)c1ccc(-c2ccccc2)cc1 ; NC1CC2CCC(C1)N2C(=O)C(NS(=O)(=O)c1ccc2cc(OC3CCCC3)ccc2c1)C(F)(F)c1ccc(-c2ccccc2)cc1 ; "CF3COOH"]

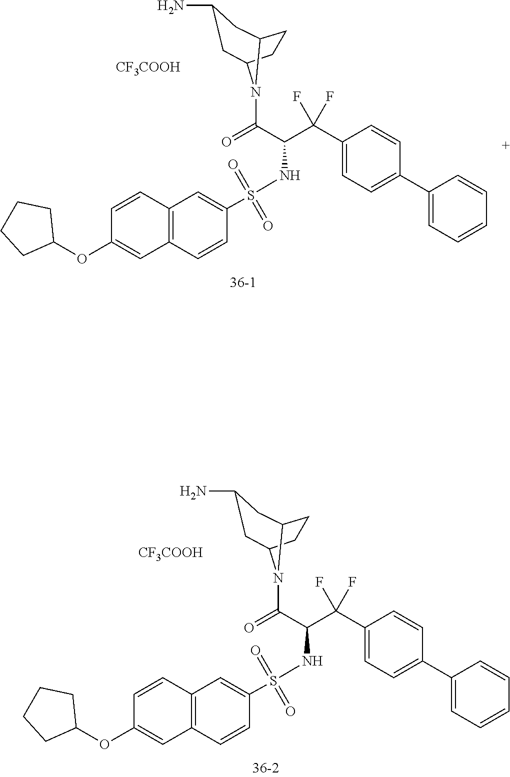 [SMILES: NC1CC2CCC(C1)N2C(=O)[C@@H](NS(=O)(=O)c1ccc2cc(OC3CCCC3)ccc2c1)C(F)(F)c1ccc(-c2ccccc2)cc1 ; "CF3COOH" ; NC1CC2CCC(C1)N2C(=O)[C@H](NS(=O)(=O)c1ccc2cc(OC3CCCC3)ccc2c1)C(F)(F)c1ccc(-c2ccccc2)cc1 ; "CF3COOH"]

Compounds 36A and 36 were synthesized by employing the procedures described for Compounds 14C and 1 using phenylboronic acid, Compounds 5A, and 36A in lieu of Compounds 14B, 14A, and 1A.

Compound 36A: LC-MS (ESI) m/z: 782 [M+Na]⁺; ¹H-NMR (CDCl₃, 400 MHz): δ (ppm) 0.43-0.52 (m, 1H), 0.78-0.87 (m, 1H), 1.24-1.28 (m, 2H), 1.38-1.46 (m, 9H), 1.65-2.04 (m, 12H), 3.47-3.50 (m, 1H), 3.75-3.87 (m, 1H), 4.11-4.17 (m, 1H), 4.42-4.61 (m, 2H), 4.80-4.88 (m, 1H), 5.96-6.09 (m, 1H), 7.02-7.06 (m, 1H), 7.16-7.26 (m, 1H), 7.40-7.53 (m, 9H), 7.63-7.78 (m, 3H), 8.15-8.21 (m, 1H).

Compound 36 was separated with chiral HPLC to give Compound 36-1 and Compound 36-2.

Compound 36-1: LC-MS (ESI) m/z: 660 [M+H]⁺; ¹H-NMR (CD₃OD, 400 MHz): δ (ppm) 0.92-2.22 (m, 16H), 3.51-3.76 (m, 1H), 4.46-4.84 (m, 3H), 4.86-4.98 (m, 1H), 6.96-7.17 (m, 2H), 7.28-7.81 (m, 12H), 8.09-8.15 (m, 1H). Chiral separation condition: n-hexane/EtOH contained 0.1% DEA, 50/50; (S, S)-Whelk-O1 (4.6×250 mm, 5 μm); retention time: 13.45 minute.

Compound 36-2: LC-MS (ESI) m/z: 660 [M+H]⁺; ¹H-NMR (CD₃OD, 400 MHz): δ (ppm) 0.92-2.17 (m, 16H), 3.54-3.77 (m, 1H), 4.39-4.77 (m, 3H), 4.83-4.95 (m, 1H), 6.95-7.16 (m, 2H), 7.28-7.83 (m, 12H), 8.09-8.16 (m, 1H). Chiral separation condition: n-hexane/EtOH contained 0.1% DEA, 50/50; (S, S)-Whelk-O1 (4.6×250 mm, 5 m); retention time: 11.04 minute.

### Example 37

### Synthesis of N-((2R)-3-(3-amino-8-azabicyclo[3.2.1]octan-8-yl)-1-(4-bromophenyl)-1,1-difluoro-3-oxopropan-2-yl)-6-(cyclohexyloxy)-N-methylnaphthalene-2-sulfonamide trifluoroacetate (Compound 37-1), and N-((2S)-3-(3-amino-8-azabicyclo[3.2.1]octan-8-yl)-1-(4-bromophenyl)-1,1-difluoro-3-oxopropan-2-yl)-6-(cyclohexyloxy)-N-methylnaphthalene-2-sulfonamide trifluoroacetate (Compound 37-2)

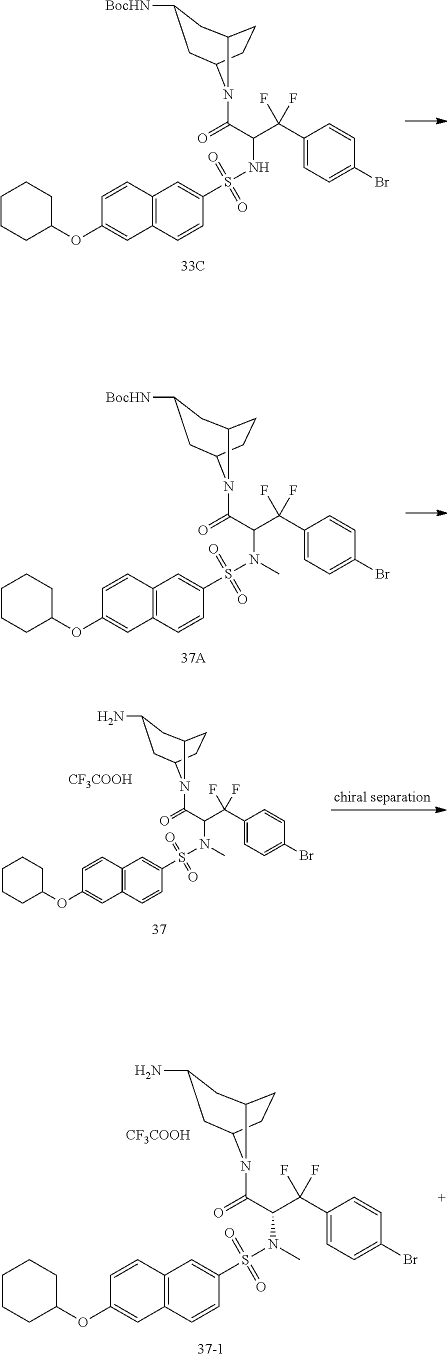

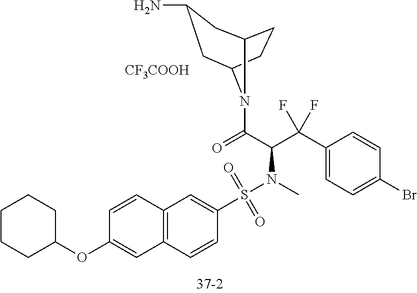 [SMILES: CN([C@@H](C(=O)N1C2CCC1CC(N)C2)C(F)(F)c1ccc(Br)cc1)S(=O)(=O)c1ccc2cc(OC3CCCCC3)ccc2c1 ; "CF3COOH"]

Compounds 37A and 37 were synthesized by employing the procedures described for Compounds 34A and 1 using Compounds 33C and 37A in lieu of Compounds 5A and 1A.

Compound 37A, which was directly used for the next step without further purification. LC-MS (ESI) m/z: 790 [M+H]⁺.

Compound 37, which was separated with chiral HPLC (MeOH contained 0.1% DEA; IC, 250×4.6 mm, 5 m) to give Compound 37-1 and Compound 37-2.

Compound 37-1: LC-MS (ESI) m/z: 690 [M+H]⁺; ¹H-NMR (CD₃OD, 400 MHz): δ (ppm) 1.30-2.22 (m, 18H), 3.05 (s, 1H), 3.23 (s, 2H), 3.64-3.74 (s, 1H), 4.56-4.61 (m, 2H), 4.67-4.74 (m, 1H), 5.55-5.61 (m, 1H), 7.28-7.56 (m, 7H), 7.78-7.86 (m, 1H), 7.89-7.96 (m, 1H), 8.18 (s, 1H). Chiral separation condition: n-hexane contained 0.1% DEA; AY-H (250×4.6 mm, 5 μm); retention time: 15.60 minute.

Compound 37-2: LC-MS (ESI) m/z: 690 [M+H]⁺; ¹H-NMR (CD₃OD, 400 MHz): δ (ppm) 1.30-2.22 (m, 18H), 3.05 (s, 1H), 3.23 (s, 2H), 3.64-3.74 (s, 1H), 4.56-4.61 (m, 2H), 4.67-4.74 (m, 1H), 5.55-5.61 (m, 1H), 7.28-7.56 (m, 7H), 7.78-7.86 (m, 1H), 7.89-7.96 (m, 1H), 8.18 (s, 1H). Chiral separation condition: n-hexane contained 0.1% DEA; AY-H (250×4.6 mm, 5 μm); retention time: 6.55 minute.

### Example 38

### Synthesis of N-((2R)-3-(3-amino-8-azabicyclo[3.2.1]octan-8-yl)-1-(6-bromonaphthalen-2-yl)-1,1-difluoro-3-oxopropan-2-yl)-6-(cyclopentyloxy) naphthalene-2-sulfonamide trifluoroacetate (Compound 38-1), and N-((2S)-3-(3-amino-8-azabicyclo[3.2.1]octan-8-yl)-1-(6-bromonaphthalen-2-yl)-1,1-difluoro-3-oxopropan-2-yl)-6-(cyclopentyloxy)naphthalene-2-sulfonamide trifluoroacetate (Compound 38-2)

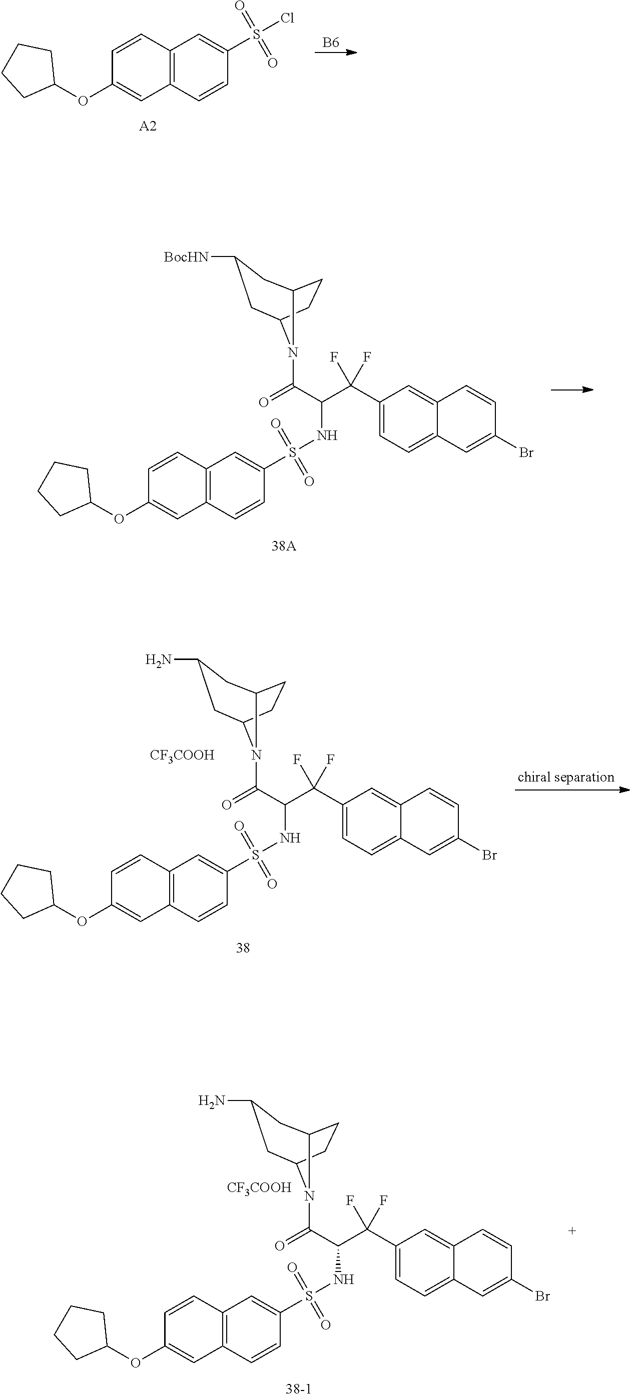

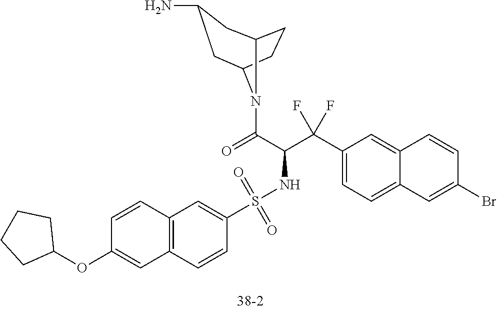 [SMILES: NC1CC2CCC(C1)N2C(=O)[C@H](NS(=O)(=O)c1ccc2cc(OC3CCCC3)ccc2c1)C(F)(F)c1ccc2cc(Br)ccc2c1]

Compounds 38A and 38 were synthesized by employing the procedures described for Compounds 2D and 1 using Compounds B6, A2, and Compound 38A in lieu of Compound B2, Compounds 2C, and 1A.

Compound 38A: LC-MS (ESI) m/z: 812 [M+H]⁺.

Compound 38 was separated with chiral HPLC to furnish Compound 38-1 and Compound 38-2.

Compound 38-1: LC-MS (ESI) m/z: 712 [M+H]⁺; ¹H-NMR (CD₃OD, 400 MHz): δ (ppm) 1.22-1.55 (m, 1H), 1.66-1.74 (m, 3H), 1.88-2.23 (m, 12H), 3.64-3.76 (m, 1H), 4.58-4.81 (m, 2H), 4.97-5.06 (m, 2H), 6.96-7.26 (m, 3H), 7.39-7.97 (m, 9H). Chiral separation condition: n-hexane/EtOH contained 0.1% DEA, 50/50; (S, S)-Whelk-O1 (4.6×250 mm, 5 μm); retention time: 15.04 minute.

Compound 38-2: LC-MS (ESI) m/z: 712 [M+H]⁺; ¹H-NMR (CD₃OD, 400 MHz): δ (ppm) 1.02-1.39 (m, 1H), 1.52-1.58 (m, 3H), 1.70-2.08 (m, 12H), 3.47-3.62 (m, 1H), 4.42-4.63 (m, 2H), 4.82-4.91 (m, 2H), 6.81-7.11 (m, 3H), 7.24-7.82 (m, 9H). Chiral separation condition: n-hexane/EtOH contained 0.1% DEA, 50/50; (S, S)-Whelk-O1 (4.6×250 mm, 5 μm); retention time: 10.87 minute.

### Example 39

### Synthesis of N-((2R)-3-(3-amino-8-azabicyclo[3.2.1]octan-8-yl)-1-(6-bromonaphthalen-2-yl)-1,1-difluoro-3-oxopropan-2-yl)-4-(cyclopentylmethoxy) benzenesulfonamide trifluoroacetate (Compound 39-1), and N-((2S)-3-(3-amino-8-azabicyclo[3.2.1]octan-8-yl)-1-(6-bromonaphthalen-2-yl)-1,1-difluoro-3-oxopropan-2-yl)-4-(cyclopentylmethoxy)benzenesulfonamide trifluoroacetate (Compound 39-2)

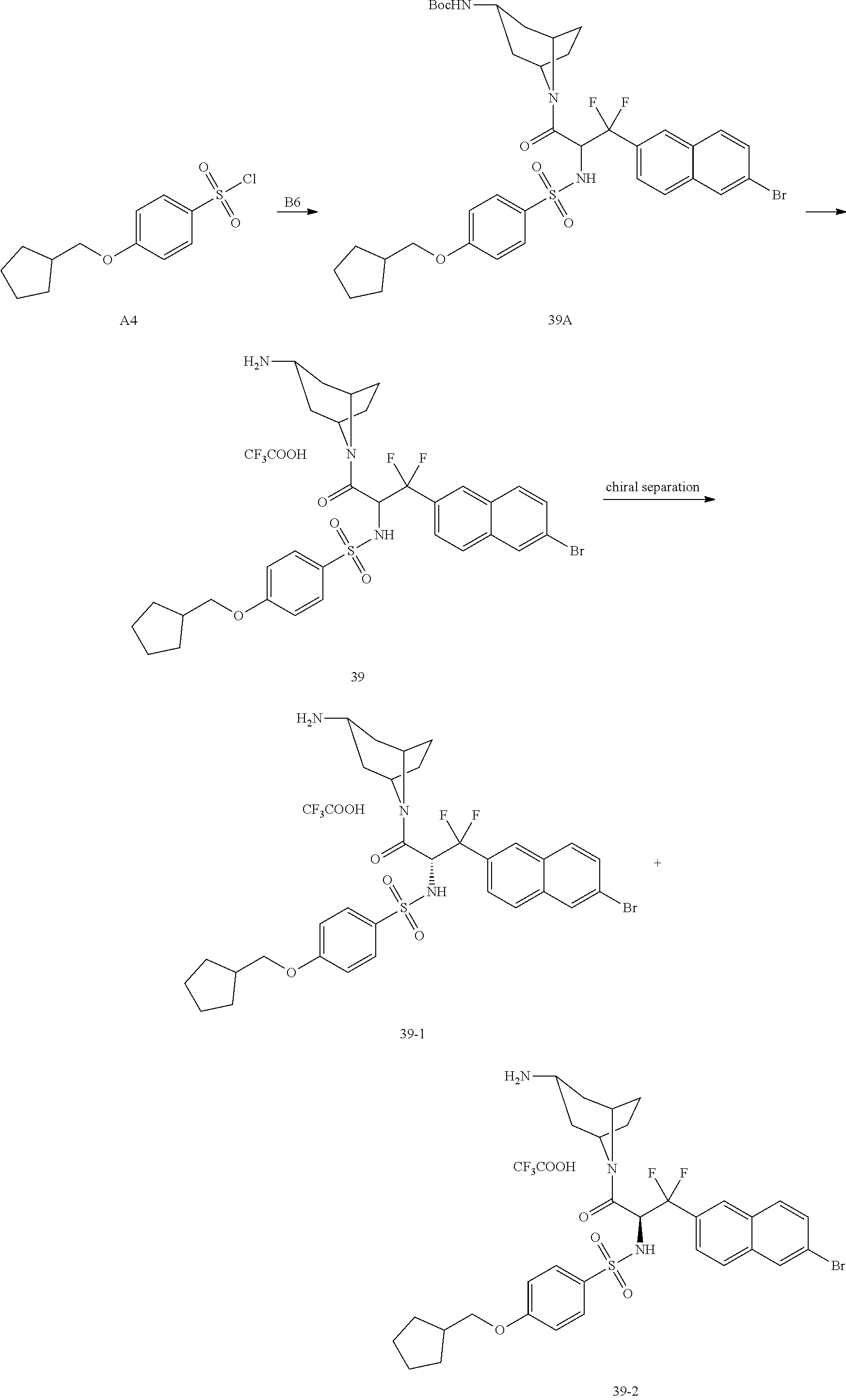

Compounds 39A and 39 were synthesized by employing the procedures described for Compounds 2D and 1 using Compounds B6, A4, and Compound 39A in lieu of Compound B2, Compounds 2C, and 1A.

Compound 39A: LC-MS (ESI) m/z: 798 [M+Na]⁺.

Compound 39 was separated with chiral HPLC to furnish Compound 39-1 and Compound 39-2.

Compound 39-1: LC-MS (ESI) m/z: 676 [M+H]⁺; ¹H-NMR (CD₃OD, 400 MHz): δ (ppm) 1.27-1.32 (m, 2H), 1.55-2.09 (m, 14H), 2.26-2.30 (m, 1H), 3.56-3.64 (m, 3H), 4.58-4.74 (m, 3H), 6.32-6.53 (m, 2H), 7.17-788 (m, 7H), 7.98-8.02 (m, 1H). Chiral separation condition: MeOH contained 0.1% NH₄OH; OJ-H (4.6×250 mm, 5 μm); retention time: 5.04 minute.

Compound 39-2: LC-MS (ESI) m/z: 676 [M+H]⁺; ¹H-NMR (CD₃OD, 400 MHz): δ (ppm) 1.37-1.43 (m, 2H), 1.62-2.20 (m, 14H), 2.33-2.40 (m, 1H), 3.69-3.76 (m, 3H), 4.69-4.85 (m, 3H), 6.43-6.64 (m, 2H), 7.28-7.99 (m, 7H), 8.09-8.13 (m, 1H). Chiral separation condition: MeOH contained 0.1% NH₄OH; OJ-H (4.6×250 mm, 5 μm); retention time: 2.53 minute.

### Example 40

### Synthesis of N-((2R)-3-(3-amino-8-azabicyclo[3.2.1]octan-8-yl)-1-(6-bromonaphthalen-2-yl)-1,1-difluoro-3-oxopropan-2-yl)-4-(cyclohexylmethoxy) benzenesulfonamide trifluoroacetate (Compound 40-1), and N-((2S)-3-(3-amino-8-azabicyclo[3.2.1]octan-8-yl)-1-(6-bromonaphthalen-2-yl)-1,1-difluoro-3-oxopropan-2-yl)-4-(cyclohexylmethoxy)benzenesulfonamide trifluoroacetate (Compound 40-2)

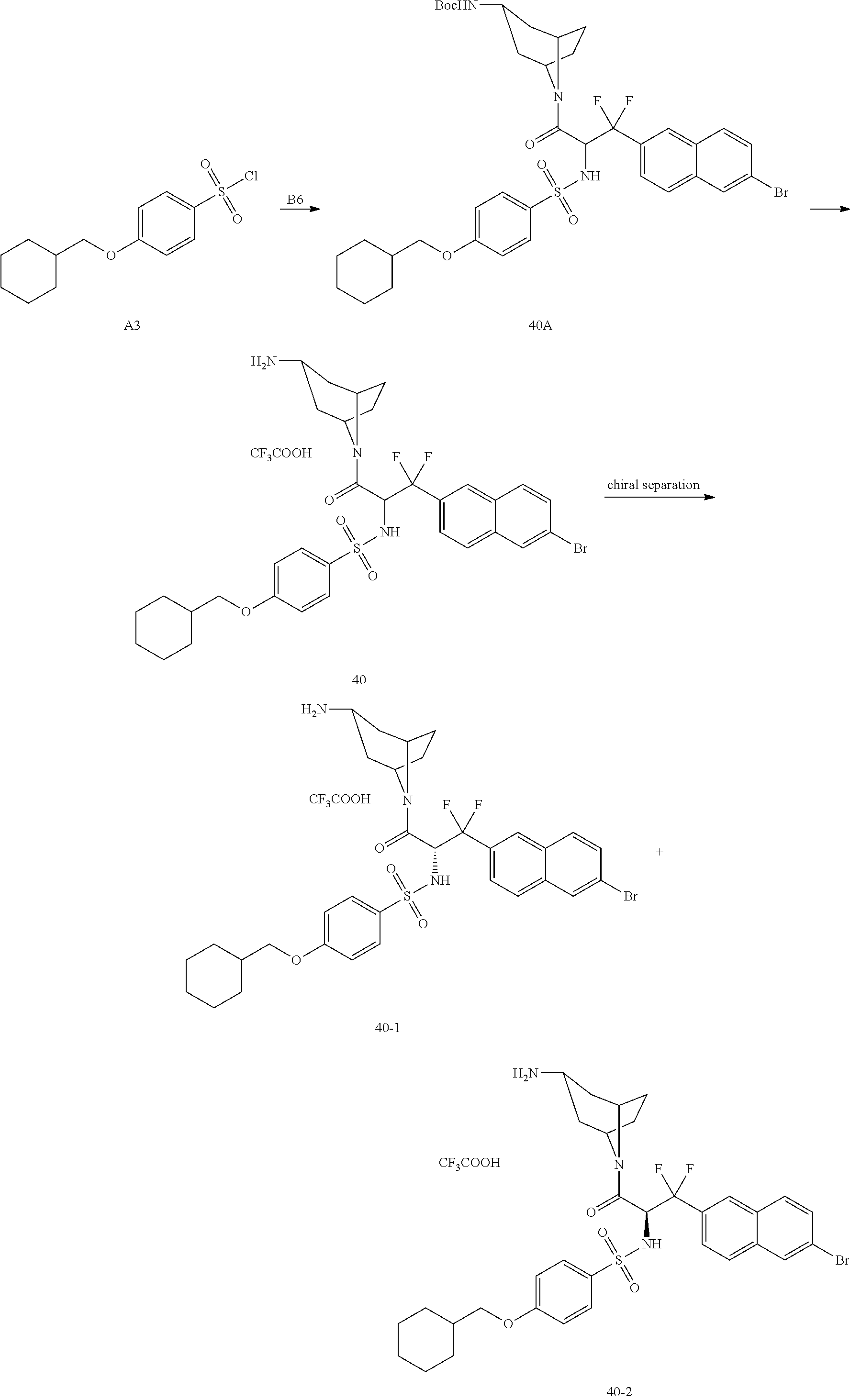

Compounds 40A and 40 were synthesized by employing the procedures described for Compounds 2D and 1 using Compounds B6, A3, and Compound 40A in lieu of Compound B2, Compounds 2C, and 1A.

Compound 40A: LC-MS (ESI) m/z: 812 [M+Na]⁺.

Compound 40 was separated with chiral HPLC to furnish Compound 40-1 and Compound 40-2.

Compound 40-1: LC-MS (ESI) m/z: 690 [M+H]⁺; ¹H-NMR (CD₃OD, 400 MHz): δ (ppm) 0.99-1.27 (m, 5H), 1.71-2.07 (m, 14H), 3.47-3.65 (m, 3H), 4.58-4.74 (m, 3H), 6.31-6.52 (m, 2H), 7.17-8.01 (m, 8H). Chiral separation condition: MeOH contained 0.1% NH₄OH; OJ-H (4.6×250 mm, 5 μm); retention time: 4.39 minute.

Compound 40-2: LC-MS (ESI) m/z: 690 [M+H]⁺; ¹H-NMR (CD₃OD, 400 MHz): δ (ppm) 1.10-1.36 (m, 5H), 1.71-2.20 (m, 14H), 3.57-3.74 (m, 3H), 4.68-4.83 (m, 3H), 6.40-6.62 (m, 2H), 7.27-8.11 (m, 8H). Chiral separation condition: MeOH contained 0.1% NH₄OH; OJ-H (4.6×250 mm, 5 μm); retention time: 2.88 minute.

### Example 41

### Synthesis of (2S)—N-((2R)-3-(3-amino-8-azabicyclo[3.2.1]octan-8-yl)-1-(4-bromophenyl)-1,1-difluoro-3-oxopropan-2-yl)-2-(6-(cyclopentyloxy)naphthalen-2-yl)-2-hydroxyacetamide trifluoroacetate (Compound 41-3), (2R)—N-((2S)-3-(3-amino-8-azabicyclo[3.2.1]octan-8-yl)-1-(4-bromophenyl)-1,1-difluoro-3-oxopropan-2-yl)-2-(6-(cyclopentyloxy)naphthalen-2-yl)-2-hydroxyacetamide trifluoroacetate (Compound 41-4), (2R)—N-((2R)-3-(3-amino-8-azabicyclo[3.2.1]octan-8-yl)-1-(4-bromophenyl)-1,1-difluoro-3-oxopropan-2-yl)-2-(6-(cyclopentyloxy)naphthalen-2-yl)-2-hydroxyacetamide trifluoroacetate (Compound 41-5), and (2S)—N-((2S)-3-(3-amino-8-azabicyclo[3.2.1]octan-8-yl)-1-(4-bromophenyl)-1,1-difluoro-3-oxopropan-2-yl)-2-(6-(cyclopentyloxy)naphthalen-2-yl)-2-hydroxyacetamide trifluoroacetate (Compound 41-6)

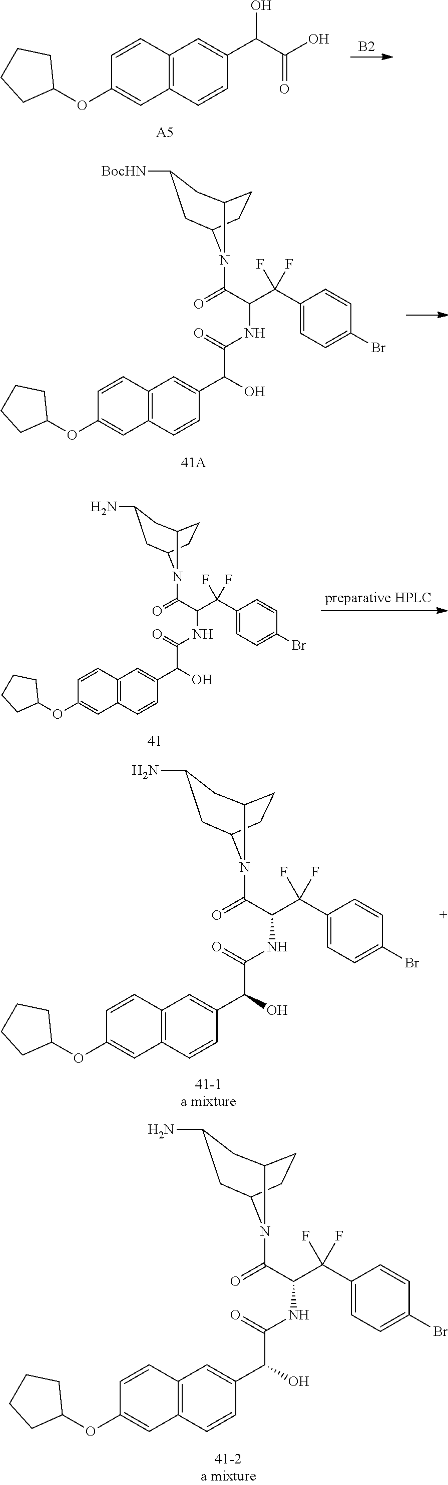

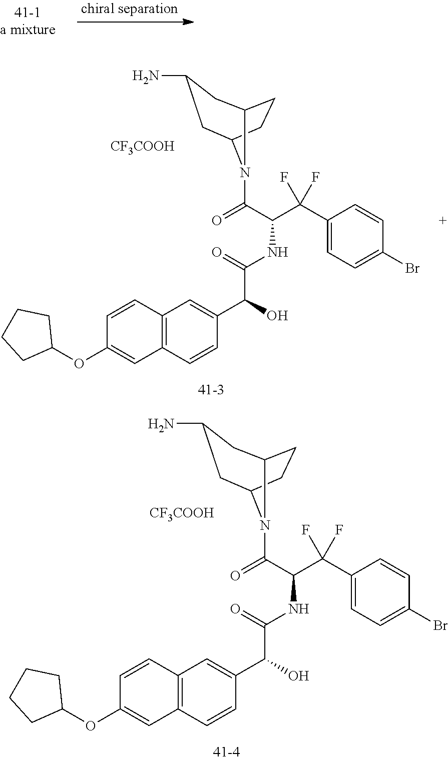 [SMILES: NC1CC2CCC(C1)N2C(=O)[C@@H](NC(=O)[C@@H](O)c1ccc2cc(OC3CCCC3)ccc2c1)C(F)(F)c1ccc(Br)cc1 ; "CF3COOH" ; NC1CC2CCC(C1)N2C(=O)[C@H](NC(=O)[C@H](O)c1ccc2cc(OC3CCCC3)ccc2c1)C(F)(F)c1ccc(Br)cc1 ; "CF3COOH"]

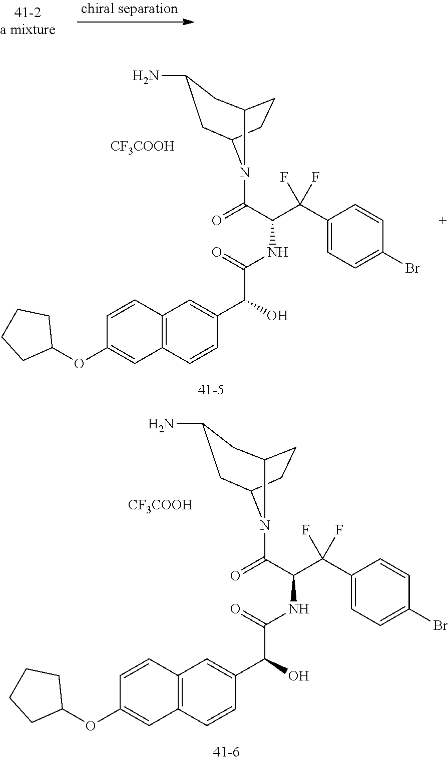 [SMILES: NC1CC2CCC(C1)N2C(=O)[C@@H](NC(=O)[C@H](O)c1ccc2cc(OC3CCCC3)ccc2c1)C(F)(F)c1ccc(Br)cc1 ; "CF3COOH" ; NC1CC2CCC(C1)N2C(=O)[C@H](NC(=O)[C@@H](O)c1ccc2cc(OC3CCCC3)ccc2c1)C(F)(F)c1ccc(Br)cc1 ; "CF3COOH"]

Compounds 41A and 41 were synthesized by employing the procedures described for Compounds 9D and 1 using Compound A5 and Compound 41A in lieu of Compounds 9C and 1A.

Compound 41A: LC-MS (ESI) m/z: 778 [M+Na]⁺.

Compound 41 was separated with preparative HPLC to afford two mixtures, Compound 41-1 and Compound 41-2, which were separated respectively with chiral HPLC to give Compound 41-3, Compound 41-4, Compound 41-5, and Compound 41-6.

Compound 41-3: LC-MS (ESI) m/z: 656 [M+H]⁺; ¹H-NMR (CD₃OD, 400 MHz): δ (ppm) 1.51-2.10 (m, 16H), 3.55-3.70 (m, 1H), 4.39-4.80 (m, 2H), 4.93-4.96 (m, 1H), 5.14-5.24 (m, 1H), 5.42-5.52 (m, 1H), 7.10-7.47 (m, 7H), 7.67-7.79 (m, 3H). Chiral separation condition: EtOH contained 0.1% DEA; IA (4.6×250 mm, 5 μm); retention time: 5.80 minute.

Compound 41-4: LC-MS (ESI) m/z: 656 [M+H]⁺; ¹H-NMR (CD₃OD, 400 MHz): δ (ppm) 1.46-2.14 (m, 16H), 3.54-3.69 (m, 1H), 4.39-4.80 (m, 2H), 4.93-4.97 (m, 1H), 5.13-5.24 (m, 1H), 5.42-5.52 (m, 1H), 7.10-7.48 (m, 7H), 7.66-7.79 (m, 3H). Chiral separation condition: EtOH contained 0.1% DEA; IA (4.6×250 mm, 5 μm); retention time: 8.66 minute.

Compound 41-5: LC-MS (ESI) m/z: 656 [M+H]⁺; ¹H-NMR (CD₃OD, 400 MHz): δ (ppm) 1.65-2.15 (m, 16H), 3.61-3.72 (m, 1H), 4.48 (s, 1H), 4.66 (s, 1H), 4.94-5.00 (m, 1H), 5.05-5.12 (m, 1H), 5.46-5.53 (m, 1H), 7.11-7.40 (m, 7H), 7.64-7.74 (m, 3H). Chiral separation condition: MeOH contained 0.5% NH₄OH; (R, R)-Whelk-O1 (4.6×250 mm, 5 μm); retention time: 4.13 minute.

Compound 41-6: LC-MS (ESI) m/z: 656 [M+H]⁺; ¹H-NMR (CD₃OD, 400 MHz): δ (ppm) 1.65-2.17 (m, 16H), 3.62-3.73 (m, 1H), 4.48 (s, 1H), 4.66 (s, 1H), 4.94-4.99 (m, 1H), 5.05-5.13 (m, 1H), 5.46-5.52 (m, 1H), 7.11-7.40 (m, 7H), 7.64-7.74 (m, 3H). Chiral separation condition: MeOH contained 0.5% NH₄OH; (R, R)-Whelk-O1 (4.6×250 mm, 5 μm); retention time: 8.09 minute.

### Example 42

### Synthesis of N-((2R)-3-(3-amino-8-azabicyclo[3.2.1]octan-8-yl)-1,1-difluoro-1-(4-iodophenyl)-3-oxopropan-2-yl)-6-(cyclopentyloxy)naphthalene-2-sulfonamide trifluoroacetate (Compound 42-1), and N-((2S)-3-(3-amino-8-azabicyclo[3.2.1]octan-8-yl)-1,1-difluoro-1-(4-iodophenyl)-3-oxopropan-2-yl)-6-(cyclopentyloxy) naphthalene-2-sulfonamide trifluoroacetate (Compound 42-2)

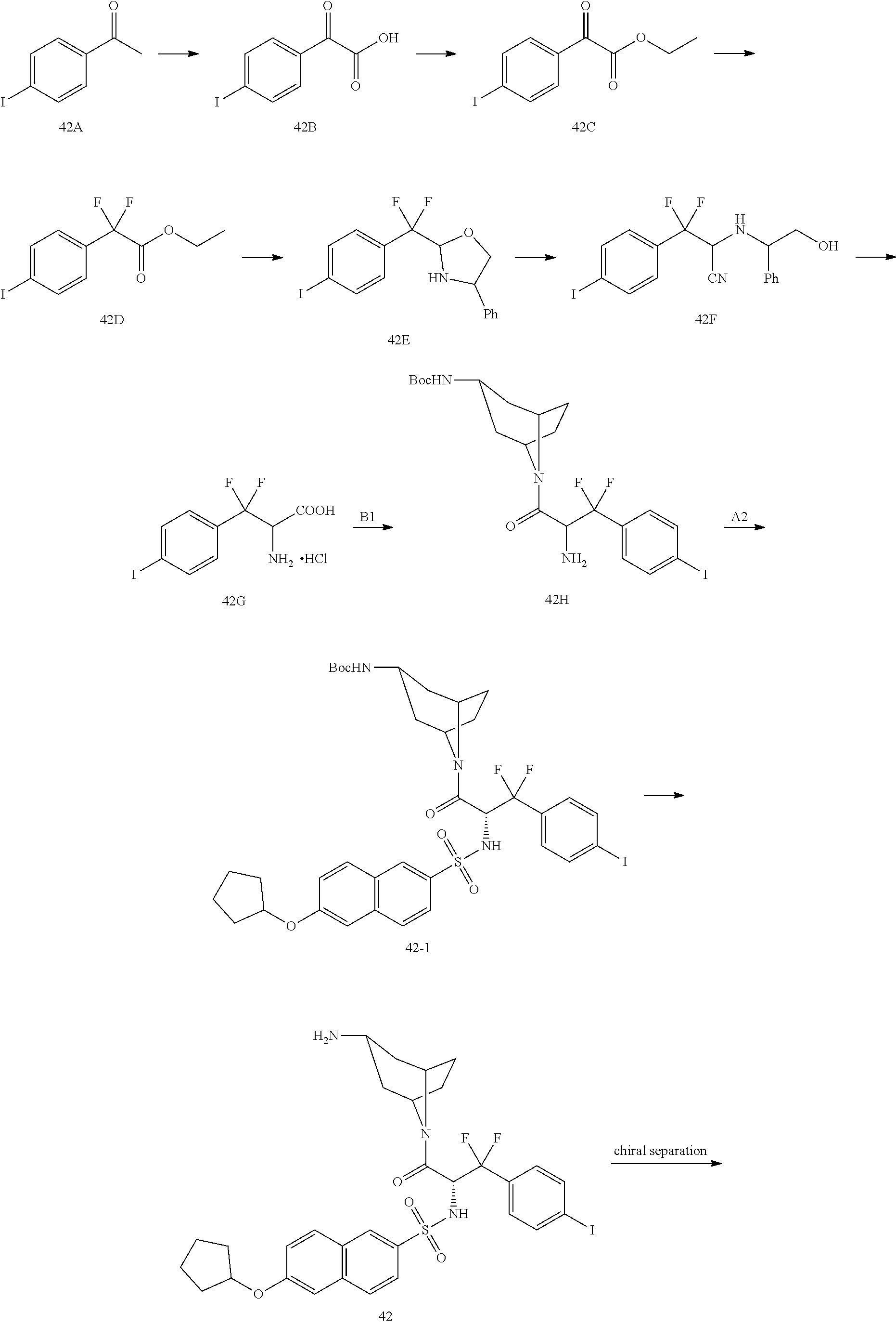

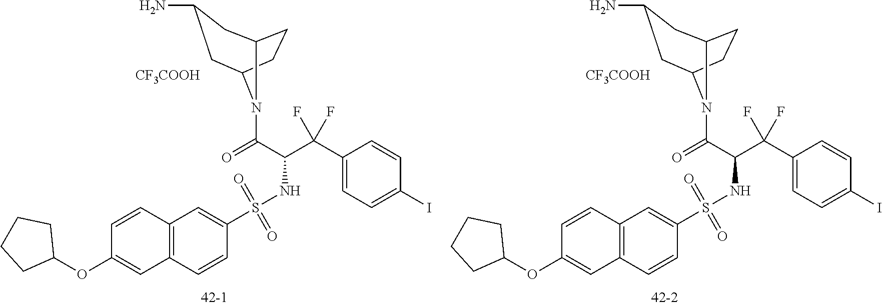

A mixture of Compound 42A (25 g, 101 mmol) and SeO₂ (16.8 g, 151 mmol) in pyridine (200 mL) was stirred at 95° C. for 16 hours. The reaction mixture was cooled down to room temperature, filtered, and concentrated to remove pyridine. To the residue was added aqueous 5% sodium hydroxide solution (200 mL) and extracted with ethyl acetate (200 mL×2). The aqueous phase was acidified to pH 2 with concentrated HCl (10 mL) and solid was formed. The solid was obtained by filtration and dried to give Compound 42B, which was used for the next step without further purification. ¹H-NMR (DMSO-d₆, 400 MHz): δ (ppm) 7.61 (d, J=8 Hz, 2H), 7.90 (d, J=8.4 Hz, 2H).

To a solution of Compound 42B (10 g, 36 mmol) in ethanol (200 mL) was added concentrated H₂SO₄ (2 mL). The reaction mixture was stirred at reflux overnight. After removal of solvent, the reaction mixture was quenched with water (100 mL) and extracted with ethyl acetate (200 mL×2). The combined organic extracts was washed with water (100 mL) and brine (100 mL), dried over anhydrous sodium sulfate, filtered, and concentrated. The crude compound was purified by silica gel column chromatography to afford Compound 42C. LC-MS (ESI) m/z: 305 [M+H]⁺; ¹H-NMR (CDCl₃, 400 MHz): δ (ppm) 1.4 (t, J=7.2 Hz, 3H), 4.5 (q, J=7.2 Hz, 2H), 7.7 (d, J=8.8 Hz, 2H), 7.9 (d, J=8.4 Hz, 2H).

Compounds 42D, 42E, 42F, 42G, 42H, 42I, and 42 were synthesized by employing the procedures described for Compounds B2-3, B2-4, B2-5, B2-6, B2, Compounds 1A, and 1 using Compounds 42C, 42D, 42E, 42F, 42G, 42H, Compound A2, and 421 in lieu of Compounds B2-2, B2-3, B2-4, B2-5, B2-6, B2, A1, and Compound 1A.

Compound 42D: LC-MS (ESI) m/z: 327 [M+H]⁺; ¹H-NMR (CDCl₃, 400 MHz): δ (ppm) 1.30 (t, J=7.2 Hz, 3H), 4.31 (q, J=7.2 Hz, 2H), 7.34 (d, J=8.0 Hz, 2H), 7.81 (d, J=8.8 Hz, 2H).

Compound 42E: LC-MS (ESI) m/z: 402 [M+H]⁺; ¹H-NMR (CDCl₃, 400 MHz): δ (ppm) 2.52-2.60 (m, 1H), 3.36-3.62 (m, 1H), 3.98-4.45 (m, 2H), 5.09-5.17 (m, 1H), 7.22-7.33 (m, 7H), 7.78 (d, J=8.0 Hz, 2H).

Compound 42F: LC-MS (ESI) m/z: 429 [M+H]⁺; ¹H-NMR (CDCl₃, 400 MHz): δ (ppm) 3.44-3.51 (m, 1H), 3.62-3.78 (m, 2H), 3.99-4.03 (m, 1H), 6.70 (d, J=7.6 Hz, 2H), 7.19-7.26 (m, 5H), 7.81 (d, J=8.4 Hz, 2H).

Compound 42G: LC-MS (ESI) m/z: 328 [M+H]⁺; ¹H-NMR (DMSO-d₆, 400 MHz): δ (ppm) 5.02-5.07 (m, 1H), 7.13-7.26 (m, 2H), 7.39 (d, J=8.8 Hz, 2H), 7.93 (d, J=8.4 Hz, 2H), 8.80-9.43 (m, 2H).

Compound 42H: LC-MS (ESI) m/z: 536 [M+H]⁺.

Compound 421: LC-MS (ESI) m/z: 810 [M+H]⁺.

Compound 42 was separated with chiral HPLC to yield Compound 42-1 and Compound 42-2.

Compound 42-1: LC-MS (ESI) m/z: 710 [M+H]⁺; ¹H-NMR (CD₃OD, 400 MHz): δ (ppm) 1.58-2.04 (m, 16H), 3.53-3.71 (m, 1H), 4.37-4.71 (m, 3H), 5.01-5.04 (m, 1H), 7.03 (d, J=8.4 Hz, 1H), 7.23-7.33 (m, 3H), 7.40-7.55 (m, 2H), 7.69-7.87 (m, 3H), 8.09-8.17 (m, 1H). Chiral separation condition: n-hexane/EtOH contained 0.1% NH₄OH, 50/50; (S,S)-WHELK-O1 (4.6×250 mm, 5 μm); retention time: 11.13 minute.

Compound 42-2: LC-MS (ESI) m/z: 710 [M+H]⁺; ¹H-NMR (CD₃OD, 400 MHz): δ (ppm) 1.40-2.17 (m, 16H), 3.52-3.72 (m, 1H), 4.37-4.83 (m, 3H), 5.01-5.04 (m, 1H), 7.03 (d, J=8.4 Hz, 1H), 7.23-7.33 (m, 3H), 7.42-7.53 (m, 2H), 7.71-7.87 (m, 3H), 8.08-8.17 (m, 1H). Chiral separation condition: n-hexane/EtOH contained 0.1% NH₄OH, 50/50; (S,S)-WHELK-O1 (4.6×250 mm, 5 μm); retention time: 9.51 minute.

### Example 43

### Synthesis of (R)—N-(1-([1,1′-biphenyl]-4-yl)-3-(4-aminopiperidin-1-yl)-1,1-difluoro-3-oxopropan-2-yl)-6-(cyclopentyloxy)naphthalene-2-sulfonamide (Compound 43-1), and (S)—N-(1-([1,1′-biphenyl]-4-yl)-3-(4-aminopiperidin-1-yl)-1,1-difluoro-3-oxopropan-2-yl)-6-(cyclopentyloxy)naphthalene-2-sulfonamide (Compound 43-2)

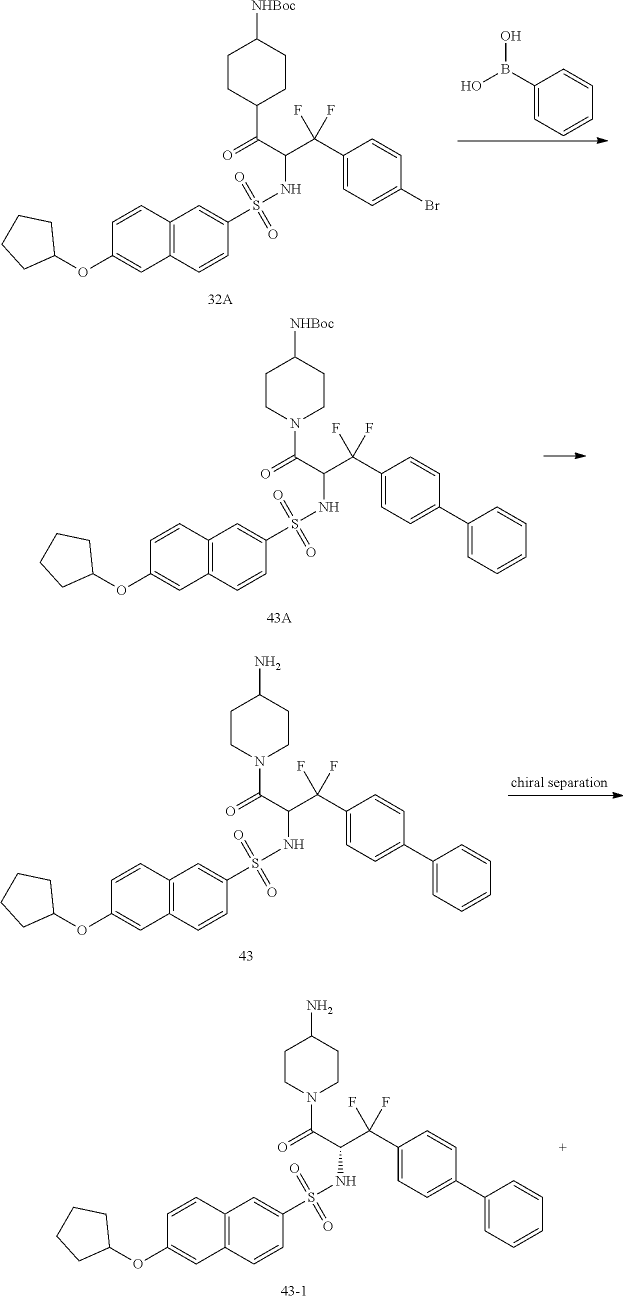 [SMILES: CC(C)(C)OC(=O)NC1CCC(C(=O)C(NS(=O)(=O)c2ccc3cc(OC4CCCC4)ccc3c2)C(F)(F)c2ccc(Br)cc2)CC1 ; OB(O)c1ccccc1 ; CC(C)(C)OC(=O)NC1CCN(C(=O)C(NS(=O)(=O)c2ccc3cc(OC4CCCC4)ccc3c2)C(F)(F)c2ccc(-c3ccccc3)cc2)CC1 ; NC1CCN(C(=O)C(NS(=O)(=O)c2ccc3cc(OC4CCCC4)ccc3c2)C(F)(F)c2ccc(-c3ccccc3)cc2)CC1 ; NC1CCN(C(=O)[C@@H](NS(=O)(=O)c2ccc3cc(OC4CCCC4)ccc3c2)C(F)(F)c2ccc(-c3ccccc3)cc2)CC1 ; "+"]

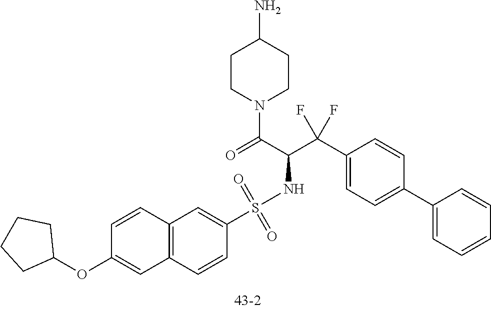 [SMILES: NC1CCN(C(=O)[C@H](NS(=O)(=O)c2ccc3cc(OC4CCCC4)ccc3c2)C(F)(F)c2ccc(-c3ccccc3)cc2)CC1]

Compounds 43A and 43 were synthesized by employing the procedures described for Compounds 14C and 1 using phenylboronic acid, Compounds 32A, and 43A in lieu of Compounds 14B, 14A, and 1A.

Compound 43A: LC-MS (ESI) m/z: 678 [M−55]⁺.

Compound 43 was separated with chiral HPLC to furnish Compound 43-1 and Compound 43-2.

Compound 43-1: LC-MS (ESI) m/z: 634 [M+H]⁺; ¹H-NMR (CD₃OD, 400 MHz): δ (ppm) 0.17-0.54 (m, 1H), 0.77-0.81 (m, 1H), 1.41-1.44 (m, 1H), 1.56-1.58 (m, 3H), 1.72-1.75 (m, 4H), 1.86-1.94 (m, 2H), 2.38-2.47 (m, 1H), 2.61-2.72 (m, 1H), 3.63-4.03 (m, 2H), 4.78-4.87 (m, 3H), 7.05-7.08 (m, 2H), 7.26-7.47 (m, 10H), 7.62-7.67 (m, 1H), 7.72-7.77 (m, 1H), 8.07-8.11 (m, 1H). Chiral separation condition: n-hexane/EtOH contained 0.1% NH₄OH, 60/40; S,S-Whelk (4.6×250 mm, 5 μm); retention time: 23.01 minute.

Compound 43-2: LC-MS (ESI) m/z: 634 [M+H]⁺; ¹H-NMR (CD₃OD, 400 MHz): δ (ppm) 0.29-0.64 (m, 1H), 0.89-0.92 (m, 1H), 1.54-1.57 (m, 1H), 1.67-1.71 (m, 3H), 1.84-1.87 (m, 4H), 2.01-2.06 (m, 2H), 2.54-2.59 (m, 1H), 2.77-2.81 (m, 1H), 3.84-4.17 (m, 2H), 4.91-4.99 (m, 3H), 7.18-7.20 (m, 2H), 7.38-7.59 (m, 10H), 7.74-7.79 (m, 1H), 7.85-7.89 (m, 1H), 8.19-8.23 (m, 1H). Chiral separation condition: n-hexane/EtOH contained 0.1% NH₄OH, 60/40; S,S-Whelk (4.6×250 mm, 5 μm); retention time: 16.93 minute.

### Example 44

### Synthesis of (R)—N-(3-(4-aminopiperidin-1-yl)-1-(4-bromophenyl)-1,1-difluoro-3-oxopropan-2-yl)-6-(cyclopentyloxy)-N-methylnaphthalene-2-sulfonamide (Compound 44-1), and (S)—N-(3-(4-aminopiperidin-1-yl)-1-(4-bromophenyl)-1,1-difluoro-3-oxopropan-2-yl)-6-(cyclopentyloxy)-N-methylnaphthalene-2-sulfonamide (Compound 44-2)

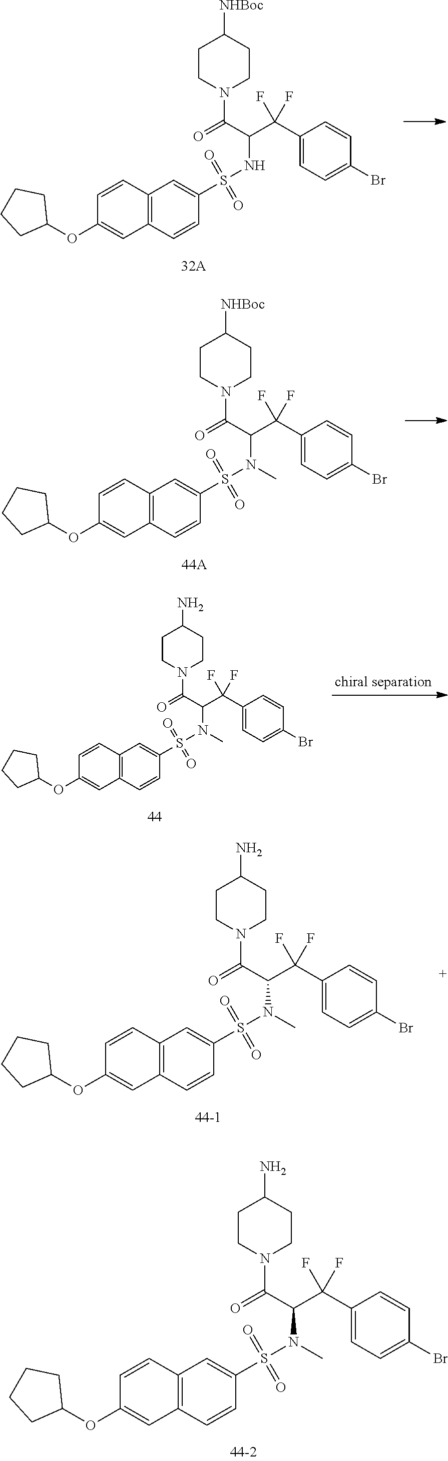 [SMILES: CC(C)(C)OC(=O)NC1CCN(C(=O)C(NS(=O)(=O)c2ccc3cc(OC4CCCC4)ccc3c2)C(F)(F)c2ccc(Br)cc2)CC1 ; CN(C(C(=O)N1CCC(NC(=O)OC(C)(C)C)CC1)C(F)(F)c1ccc(Br)cc1)S(=O)(=O)c1ccc2cc(OC3CCCC3)ccc2c1 ; CN(C(C(=O)N1CCC(N)CC1)C(F)(F)c1ccc(Br)cc1)S(=O)(=O)c1ccc2cc(OC3CCCC3)ccc2c1 ; CN([C@H](C(=O)N1CCC(N)CC1)C(F)(F)c1ccc(Br)cc1)S(=O)(=O)c1ccc2cc(OC3CCCC3)ccc2c1 ; "+" ; CN([C@@H](C(=O)N1CCC(N)CC1)C(F)(F)c1ccc(Br)cc1)S(=O)(=O)c1ccc2cc(OC3CCCC3)ccc2c1]

Compounds 44A and 44 were synthesized by employing the procedures described for Compounds 34A and 1 using Compounds 32A at room temperature and 44A in lieu of Compounds 5A at 0° C. and 1A.

Compound 44A: LC-MS (ESI) m/z: 772 [M+Na]⁺.

Compound 44 was separated with chiral HPLC to furnish Compound 44-1 and Compound 44-2.

Compound 44-1: LC-MS (ESI) m/z: 650 [M+H]⁺; ¹H-NMR (CD₃OD, 400 MHz): δ (ppm) 0.78-0.81 (m, 1H), 0.96-1.05 (m, 1H), 1.60-1.62 (m, 2H), 1.72-1.83 (m, 6H), 1.94-1.98 (m, 2H), 2.42-2.60 (m, 1H), 2.88-3.15 (m, 1H), 2.98-3.01 (m, 3H), 4.04-4.35 (m, 2H), 4.92 (s, 1H), 5.58-5.67 (m, 1H), 7.15 (d, J=8.8 Hz, 1H), 7.22 (s, 1H), 7.26 (d, J=8.4 Hz, 1H), 7.33-7.46 (m, 4H), 7.73-7.77 (m, 1H), 7.82 (d, J=8.8 Hz, 1H), 8.10-8.12 (m, 1H). Chiral separation condition: n-hexane/EtOH contained 0.1% NH₄OH, 60/40; IB (4.6×250 mm, 5 μm); retention time: 8.68 minute.

Compound 44-2: LC-MS (ESI) m/z: 650 [M+H]⁺; ¹H-NMR (CD₃OD, 400 MHz): δ (ppm) 0.89-0.92 (m, 1H), 1.02-1.15 (m, 1H), 1.70-1.73 (m, 2H), 1.82-1.94 (m, 6H), 2.04-2.09 (m, 2H), 2.52-2.70 (m, 1H), 2.94-3.25 (m, 1H), 3.09-3.12 (m, 3H), 4.12-4.43 (m, 2H), 5.02 (s, 1H), 5.68-5.78 (m, 1H), 7.26 (d, J=9.2 Hz, 1H), 7.32 (s, 1H), 7.37 (d, J=8.4 Hz, 1H), 7.44-7.57 (m, 4H), 7.84-7.87 (m, 1H), 7.92 (d, J=9.2 Hz, 1H), 8.21-8.23 (m, 1H). Chiral separation condition: n-hexane/EtOH contained 0.1% NH₄OH, 60/40; IB (4.6×250 mm, 5 μm); retention time: 9.85 minute.

### Example 45

### Synthesis of N-((2R)-3-(3-amino-8-azabicyclo[3.2.1]octan-8-yl)-1,1-difluoro-1-(4-methoxyphenyl)-3-oxopropan-2-yl)-6-(cyclopentyloxy)naphthalene-2-sulfonamide (Compound 45-1), and N-((2S)-3-(3-amino-8-azabicyclo[3.2.1]octan-8-yl)-1,1-difluoro-1-(4-methoxyphenyl)-3-oxopropan-2-yl)-6-(cyclopentyloxy)naphthalene-2-sulfonamide (Compound 45-2)

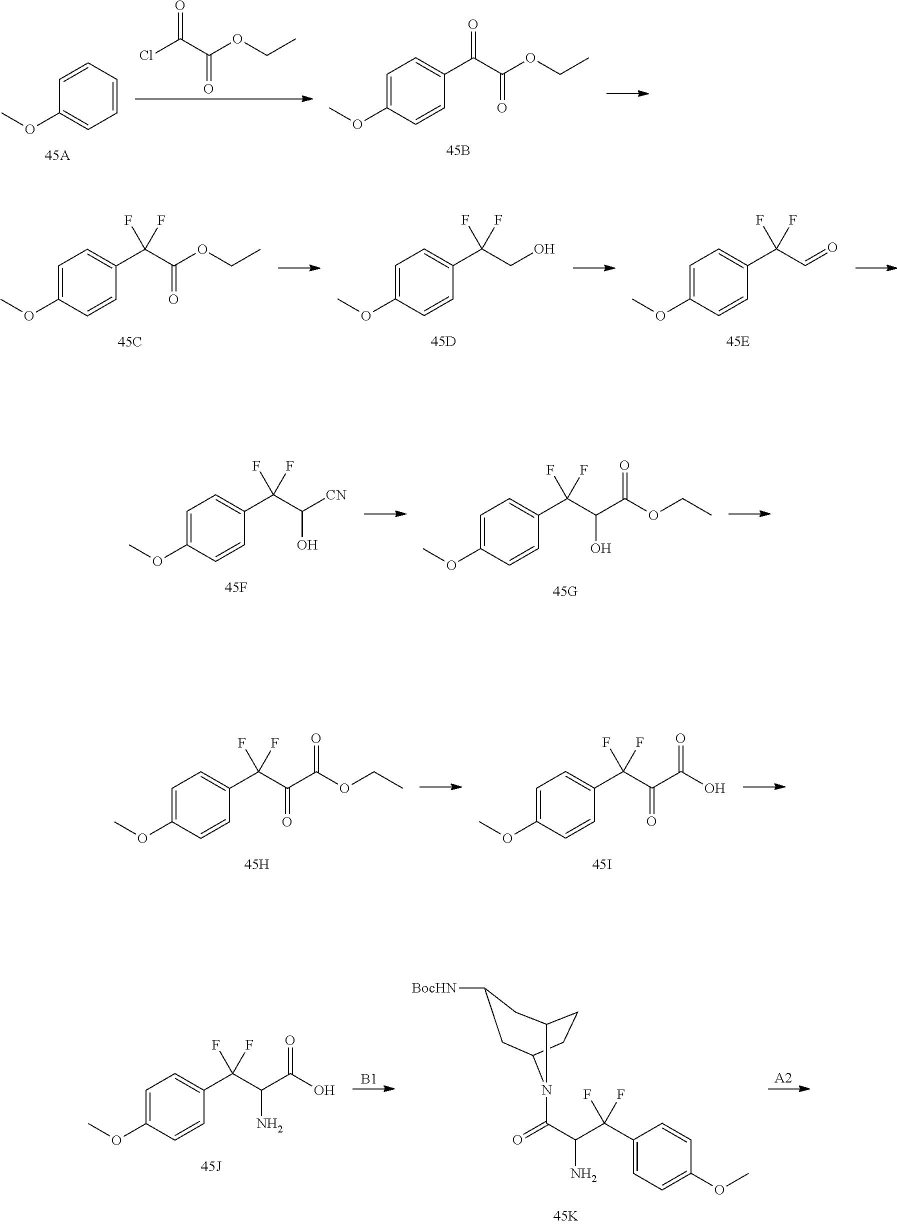

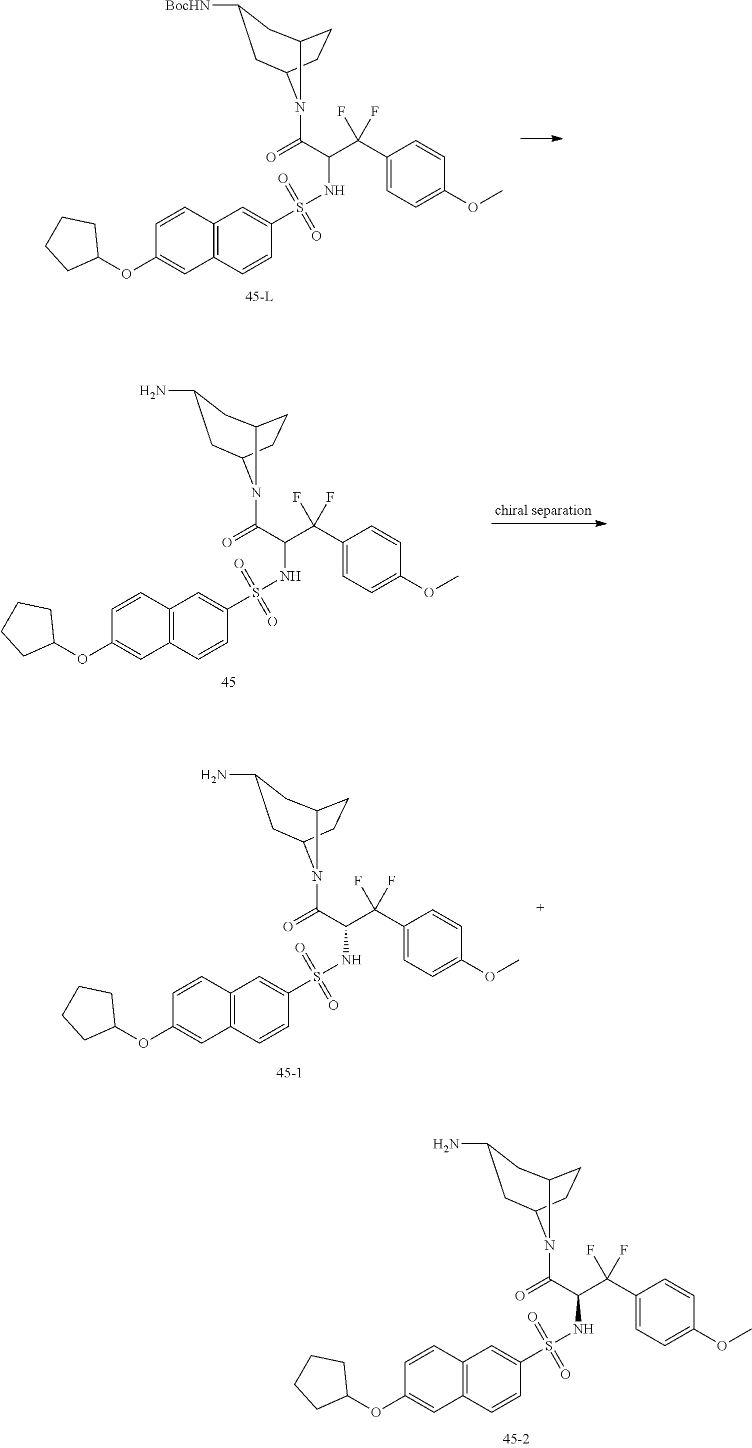 [SMILES: COc1ccc(C(F)(F)C(NS(=O)(=O)c2ccc3cc(OC4CCCC4)ccc3c2)C(=O)N2C3CCC2CC(NC(=O)OC(C)(C)C)C3)cc1 ; COc1ccc(C(F)(F)C(NS(=O)(=O)c2ccc3cc(OC4CCCC4)ccc3c2)C(=O)N2C3CCC2CC(N)C3)cc1 ; COc1ccc(C(F)(F)[C@H](NS(=O)(=O)c2ccc3cc(OC4CCCC4)ccc3c2)C(=O)N2C3CCC2CC(N)C3)cc1 ; COc1ccc(C(F)(F)[C@@H](NS(=O)(=O)c2ccc3cc(OC4CCCC4)ccc3c2)C(=O)N2C3CCC2CC(N)C3)cc1]

Compounds 45B and 45C were synthesized by employing the procedures described for Compounds B2-2 and B2-3 using Compounds 45A and 45B in lieu of Compounds B2-1 and B2-2.

Compound 45B: LC-MS (ESI) m/z: 209 [M+H]⁺; ¹H-NMR (CDCl₃, 400 MHz): δ (ppm) 1.42 (t, J=7.2 Hz, 3H), 3.89 (s, 3H), 4.41-4.46 (m, 2H), 6.97 (d, J=11.6 Hz, 2H), 8.00 (d, J=11.6 Hz, 2H).

Compound 45C: ¹H-NMR (CDCl₃, 400 MHz): δ (ppm) 1.30 (t, J=7.2 Hz, 3H), 3.84 (s, 3H), 4.26-4.32 (m, 2H), 6.95 (d, J=8.8 Hz, 2H), 7.53 (d, J=8.8 Hz, 2H).

To a solution of AlLiH₄ (2.97 g, 78 mmol) in THF (100 mL) was dropped a solution of Compound 45C (9 g, 39 mmol) in THF (20 mL) under nitrogen at −10° C. The reaction mixture was stirred at 0° C. for 2 hours, quenched with Na₂SO₄.10H₂O, and filtered. The filtrate was concentrated to afford a crude Compound 45D.

Compound 45E was synthesized by employing the procedure described for Compound B6-3 using Compound 45D in lieu of Compound B6-2, which was unstable and used directly for the next step.

To a solution of Na₂S₂O₅ (570 mg, 3 mmol) in water (5 mL) was added Compound 45E (558 mg, 3 mmol). After it was stirred at room temperature for 2 hours, to it was added NaCN (294 mg, 6 mmol), stirred for 15 hours, and extracted with ethyl acetate (20 mL×2). The combined organic extracts were washed with saturated sodium bicarbonate solution (20 mL×2) and brine (40 mL), dried over anhydrous sodium sulfate, filtered, and concentrated to afford a crude product. It was purified by silica gel column chromatography to give Compound 45F. LC-MS (ESI) m/z: 196 [M−OH]⁺; ¹H-NMR

(CDCl₃, 400 MHz): δ (ppm) 3.33 (d, J=8.4 Hz, 1H), 3.85 (s, 3H), 4.75-4.81 (m, 1H), 6.79 (d, J=8.8 Hz, 2H), 7.50 (d, J=8.8 Hz, 2H).

To a solution of Compound 45F (2.41 g, 11 mmol) in ethanol (30 mL) at 0° C. was bubbled a gentle stream of HCl gas, which was dried over con. H₂SO₄, for 3 hours. The reaction mixture was kept at 4° C. overnight. To it was slowly added water at 0° C., stirred at room temperature for 2 hours, and extracted with dichloromethane (100 mL×2). The combined organic extracts was washed with brine (100 mL), dried over anhydrous sodium sulfate, filtered, and concentrated. The residue was purified by silica gel column chromatography to give Compound 45G. LC-MS (ESI) m/z: 241 [M−F]⁺; ¹H-NMR (CDCl₃, 400 MHz): δ (ppm) 1.27 (t, J=7.2 Hz, 3H), 3.21 (d, J=8.0 Hz, 1H), 3.83 (s, 3H), 4.24-4.30 (m, 2H), 4.47-4.54 (m, 1H), 6.93 (d, J=9.2 Hz, 2H), 7.41 (d, J=9.2 Hz, 2H).

Compound 45H was synthesized by employing the procedure described for Compound B6-3 using Compound 45G in lieu of Compound B6-2. LC-MS (ESI) m/z: 239 [M−F]⁺; ¹H-NMR (CDCl₃, 400 MHz): δ (ppm) 1.37 (t, J=7.2 Hz, 3H), 3.83 (s, 3H), 4.35-4.40 (m, 2H), 6.93 (d, J=8.4 Hz, 2H), 7.52 (d, J=8.4 Hz, 2H).

A mixture of Compound 45H (327 mg, 1.27 mmol) and sodium bicarbonate (319 mg, 3.81 mmol) in 50% aqueous isopropanol (5 mL) was heated at 50° C. for 10 hours and concentrated under reduced pressure. The residue was dissolved in water (10 mL), washed with diethyl ether, acidified with 1.0 M hydrochloric acid solution (5 mL), saturated with sodium chloride, and extracted with diethyl ether (20×2 mL). The combined organic extracts were washed with brine (30 mL), dried over anhydrous sodium sulfate, filtered, and concentrated to give a crude Compound 451. LC-MS (ESI) m/z: 211 [M−F]⁺.

A solution of Compound 451 (230 mg, 1 mmol) in a 25% aqueous ammonia solution (2 mL) in a sealed pressure-resistant vessel was heated at 60° C. for 5 hours. To it at 25° C. was added sodium borohydride (114 mg, 3 mmol), while a gentle current of nitrogen was bubbled through it for 30 minutes. The reaction mixture was purified with preparative HPLC to afford Compound 45J. LC-MS (ESI) m/z: 232 [M+H]⁺; ¹H-NMR (DMSO-d₆, 400 MHz): δ (ppm) 3.78 (s, 3H), 3.80-3.90 (m, 1H), 6.96 (d, J=8.8 Hz, 2H), 7.42 (d, J=8.8 Hz, 2H).

Compounds 45K, 45L, and 45 were synthesized by employing the procedures described for Compound B2, Compounds 2D and 1 using Compounds 45J, 45K, and 45L in lieu of Compound B2-6, Compounds 2C, and 1A.

Compound 45K: LC-MS (ESI) m/z: 440 [M+H]⁺; ¹H-NMR (CD₃OD, 400 MHz): δ (ppm) 1.03-1.17 (m, 1H), 1.40-1.85 (m, 16H), 3.82-4.00 (m, 5H), 4.22-4.36 (m, 1H), 4.44-4.65 (m, 1H), 6.97-7.03 (m, 2H), 7.37-7.45 (m, 2H).

Compound 45K: LC-MS (ESI) m/z: 736 [M+Na]⁺.

Compound 45, which was separated with chiral HPLC (MeOH contained 0.1% NH₄OH; OJ-H, 250×4.6 mm, 5 m) to give Compound 45-1 and Compound 45-2.

Compound 45-1: LC-MS (ESI) m/z: 614 [M+H]⁺; ¹H-NMR (CD₃OD, 400 MHz): δ (ppm) 0.46-0.90 (m, 2H), 1.11-1.49 (m, 2H), 1.52-1.71 (m, 6H), 1.78-1.91 (m, 4H), 1.99-2.01 (m, 2H), 2.73-2.99 (m, 1H), 3.76 (d, J=8.0 Hz, 3H), 4.06-4.36 (m, 2H), 4.60-4.68 (m, 1H), 4.96-5.02 (m, 1H), 6.84-6.90 (m, 2H), 7.19-7.42 (m, 4H), 7.63-7.90 (m, 3H), 8.23 (d, J=24.6 Hz, 1H); Chiral separation condition: MeOH contained 0.1% NH₄OH; OJ-H (250×4.6 mm, 5 μm); retention time: 4.35 minute.

Compound 45-2: LC-MS (ESI) m/z: 614 [M+H]⁺; ¹H-NMR (CD₃OD, 400 MHz): δ (ppm)¹H-NMR (CD₃OD, 400 MHz): δ (ppm) 0.46-0.88 (m, 2H), 1.10-1.48 (m, 2H), 1.51-1.71 (m, 6H), 1.78-1.90 (m, 4H), 2.01-2.08 (m, 2H), 2.72-2.98 (m, 1H), 3.75 (d, J=7.2 Hz, 3H), 4.05-4.36 (m, 2H), 4.60-4.69 (m, 1H), 4.95-4.99 (m, 1H), 6.84-6.90 (m, 2H), 7.19-7.42 (m, 4H), 7.63-7.89 (m, 3H), 8.23 (d, J=24.6 Hz, 1H); Chiral separation condition: MeOH contained 0.1% NH₄OH; OJ-H (250×4.6 mm, 5 μm); retention time: 5.77 minute.

### Example 46

### Synthesis of N-((2R)-3-(3-amino-8-azabicyclo[3.2.1]octan-8-yl)-1-(4-bromophenyl)-1,1-difluoro-3-oxopropan-2-yl)-6-isopropoxy-N-methylnaphthalene-2-sulfonamide trifluoroacetate (Compound 46-1), and N-((2S)-3-(3-amino-8-azabicyclo[3.2.1]octan-8-yl)-1-(4-bromophenyl)-1,1-difluoro-3-oxopropan-2-yl)-6-isopropoxy-N-methylnaphthalene-2-sulfonamide trifluoroacetate (Compound 46-2)

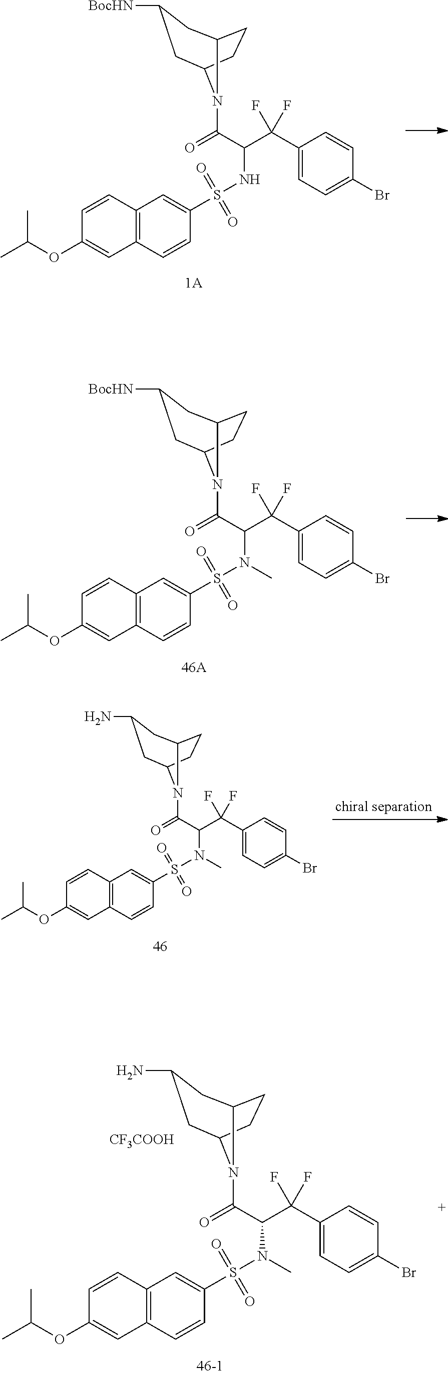 [SMILES: CC(C)Oc1ccc2cc(S(=O)(=O)NC(C(=O)N3C4CCC3CC(NC(=O)OC(C)(C)C)C4)C(F)(F)c3ccc(Br)cc3)ccc2c1 ; CC(C)Oc1ccc2cc(S(=O)(=O)N(C)C(C(=O)N3C4CCC3CC(NC(=O)OC(C)(C)C)C4)C(F)(F)c3ccc(Br)cc3)ccc2c1 ; CC(C)Oc1ccc2cc(S(=O)(=O)N(C)C(C(=O)N3C4CCC3CC(N)C4)C(F)(F)c3ccc(Br)cc3)ccc2c1 ; CC(C)Oc1ccc2cc(S(=O)(=O)N(C)[C@H](C(=O)N3C4CCC3CC(N)C4)C(F)(F)c3ccc(Br)cc3)ccc2c1 ; "CF3COOH" ; "+"]

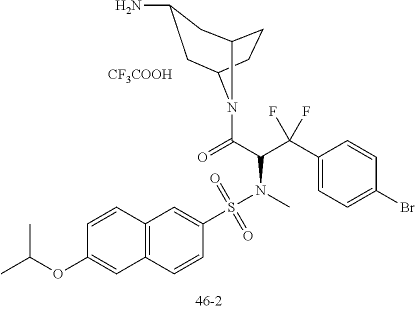

Compounds 46A and 46 were synthesized by employing the procedures described for Compounds 34A and 1 using Compounds 1A and 46A in lieu of Compounds 5A and 1A.

Compound 46A: LC-MS (ESI) m/z: 750 [M+H]⁺; ¹H-NMR (CD₃OD, 400 MHz): δ (ppm) 1.32 (d, J=6.0 Hz, 6H), 1.38 (s, 9H), 1.55-2.12 (m, 8H), 2.95 (s, 1H), 3.13 (s, 2H), 3.56-3.61 (m, 1H), 4.55-4.78 (m, 3H), 5.50-5.58 (m, 1H), 7.17-7.49 (m, 7H), 7.66-7.88 (m, 2H), 8.11-8.18 (m, 1H).

Compound 46 was separated with chiral HPLC to give Compound 46-1 and Compound 46-2.

Compound 46-1: LC-MS (ESI) m/z: 650 [M+H]⁺; ¹H-NMR (CD₃OD, 400 MHz): δ (ppm) 1.32 (d, J=6.0 Hz, 6H), 1.53-2.10 (m, 8H), 2.95 (s, 1H), 3.13 (s, 2H), 3.56-3.61 (m, 1H), 4.48-4.71 (m, 3H), 5.43-5.50 (m, 1H), 7.15-7.46 (m, 7H), 7.66-7.82 (m, 2H), 8.01-8.15 (m, 1H). Chiral separation condition: n-hexane/EtOH contained 0.1% DEA, 50/50; (S, S)-Whelk-O1 (4.6×250 mm, 5 μm); retention time: 5.55 minute.

Compound 46-2: LC-MS (ESI) m/z: 650 [M+H]⁺; ¹H-NMR (CD₃OD, 400 MHz): δ (ppm) 1.32 (d, J=6.0 Hz, 6H), 1.33-2.10 (m, 8H), 2.95 (s, 1H), 3.13 (s, 2H), 3.56-3.61 (m, 1H), 4.48-4.72 (m, 2H), 4.82 (s, 1H), 5.43-5.50 (m, 1H), 7.152-7.36 (m, 7H), 7.67-7.84 (m, 2H), 8.01-8.14 (m, 1H). Chiral separation condition: n-hexane/EtOH contained 0.1% DEA, 50/50; (S, S)-Whelk-O1 (4.6×250 mm, 5 μm); retention time: 6.37 minute.

### Example 47

### Synthesis of N-((2R)-3-(3-amino-8-azabicyclo[3.2.1]octan-8-yl)-1-(4-bromophenyl)-1,1-difluoro-3-oxopropan-2-yl)-6-cyclobutoxynaphthalene-2-sulfonamide (Compound 47-1), and N-((2S)-3-(3-amino-8-azabicyclo[3.2.1]octan-8-yl)-1-(4-bromophenyl)-1,1-difluoro-3-oxopropan-2-yl)-6-cyclobutoxynaphthalene-2-sulfonamide (Compound 47-2)

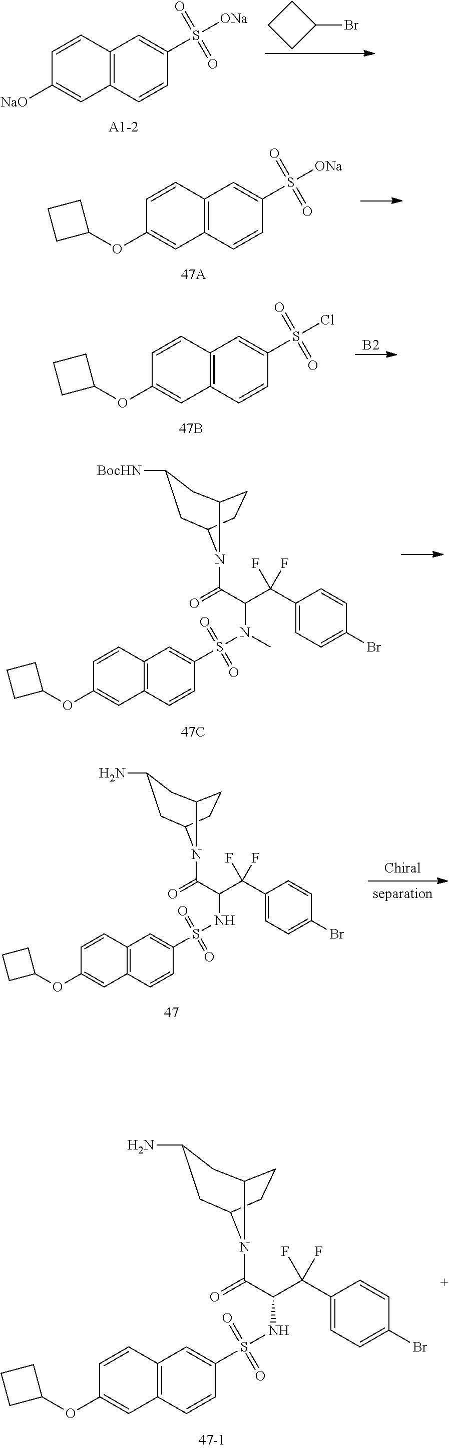

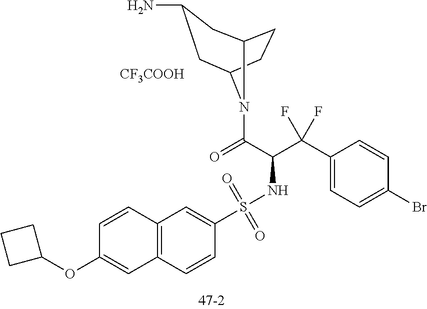 [SMILES: NC1CC2CCC(C1)N2C(=O)[C@H](NS(=O)(=O)c1ccc2cc(OC3CCC3)ccc2c1)C(F)(F)c1ccc(Br)cc1 ; "CF3COOH"]

Compounds 47A, 47B, 47C, and 47 were synthesized by employing the procedures described for Compounds A1-3, A1, Compounds 1A, and 1 using bromocyclobutane, Compounds 47A, 47B, and 47C in lieu of 2-bromopropane, Compounds A1-3, A1, and Compound 1A.

Compound 47A: LC-MS (ESI) m/z: 279 [M+2H—Na]⁺; ¹H-NMR (DMSO-d₆, 400 MHz): δ (ppm) 1.67-1.83 (m, 2H), 2.07-2.12 (m, 2H), 2.53-2.54 (m, 2H), 4.80-4.83 (m, 1H), 7.10-7.13 (m, 1H), 7.16-7.17 (m, 1H), 7.64-7.66 (m, 1H), 7.74 (d, J=8.4 Hz, 1H), 7.87 (d, J=8.8 Hz, 1H), 8.06 (s, 1H).

Compound 47B: LC-MS (ESI) m/z: 293 [M−Cl+OMe+H]⁺.

Compound 47C: LC-MS (ESI) m/z: 770 [M+Na]⁺.

Compound 47, which was separated with chiral HPLC to give Compound 47-1 and Compound 47-2.

Compound 47-1: LC-MS (ESI) m/z: 648 [M+H]⁺; ¹H-NMR (CDCl₃, 400 MHz): δ (ppm) 0.23-0.30 (m, 1H), 0.53-0.60 (m, 1H), 1.25-1.27 (m, 1H), 1.56-1.60 (m, 3H), 1.74-1.79 (m, 3H), 1.87-1.95 (m, 3H), 2.19-2.24 (m, 2H), 2.51-2.54 (m, 2H), 2.79-2.83 (m, 1H), 4.07 (s, 1H), 4.41-4.46 (m, 2H), 4.76-4.79 (m, 1H), 7.01-7.02 (m, 1H), 7.20-7.29 (m, 3H), 7.47-7.49 (m, 2H), 7.63-7.83 (m, 3H), 8.17-8.23 (m, 1H). Chiral separation condition: MeOH contained 0.1% NH₄OH; (R,R)-Whelk-O1 (4.6×250 mm, 5 μm); retention time: 4.34 minute.

Compound 47-2: LC-MS (ESI) m/z: 648 [M+H]⁺; ¹H-NMR (CDCl₃, 400 MHz): δ (ppm) 0.24-0.30 (m, 1H), 0.52-0.57 (m, 1H), 1.08-1.28 (m, 2H), 1.55-1.60 (m, 3H), 1.79-1.82 (m, 3H), 2.04-2.05 (m, 3H), 2.18-2.23 (m, 2H), 2.51-2.54 (m, 2H), 2.97-3.00 (m, 1H), 4.06 (s, 1H), 4.41-4.46 (m, 2H), 4.76-4.79 (m, 1H), 7.01-7.02 (m, 1H), 7.20-7.29 (m, 3H), 7.47-7.49 (m, 2H), 7.64-7.83 (m, 3H), 8.17-8.24 (m, 1H). Chiral separation condition: MeOH contained 0.1% NH₄OH; (R,R)-Whelk-O1 (4.6×250 mm, 5 μm); retention time: 5.26 min.

### Example 48

### Synthesis of N-((2R)-3-(3-amino-8-azabicyclo[3.2.1]octan-8-yl)-1-(4′-chloro-[1,1′-biphenyl]-4-yl)-1,1-difluoro-3-oxopropan-2-yl)-6-(cyclopentyloxy)naphthalene-2-sulfonamide trifluoroacetate (Compound 48-1), and N-((2S)-3-(3-amino-8-azabicyclo[3.2.1]octan-8-yl)-1-(4′-chloro-[1,1′-biphenyl]-4-yl)-1,1-difluoro-3-oxopropan-2-yl)-6-(cyclopentyloxy)naphthalene-2-sulfonamide trifluoroacetate (Compound 48-2)

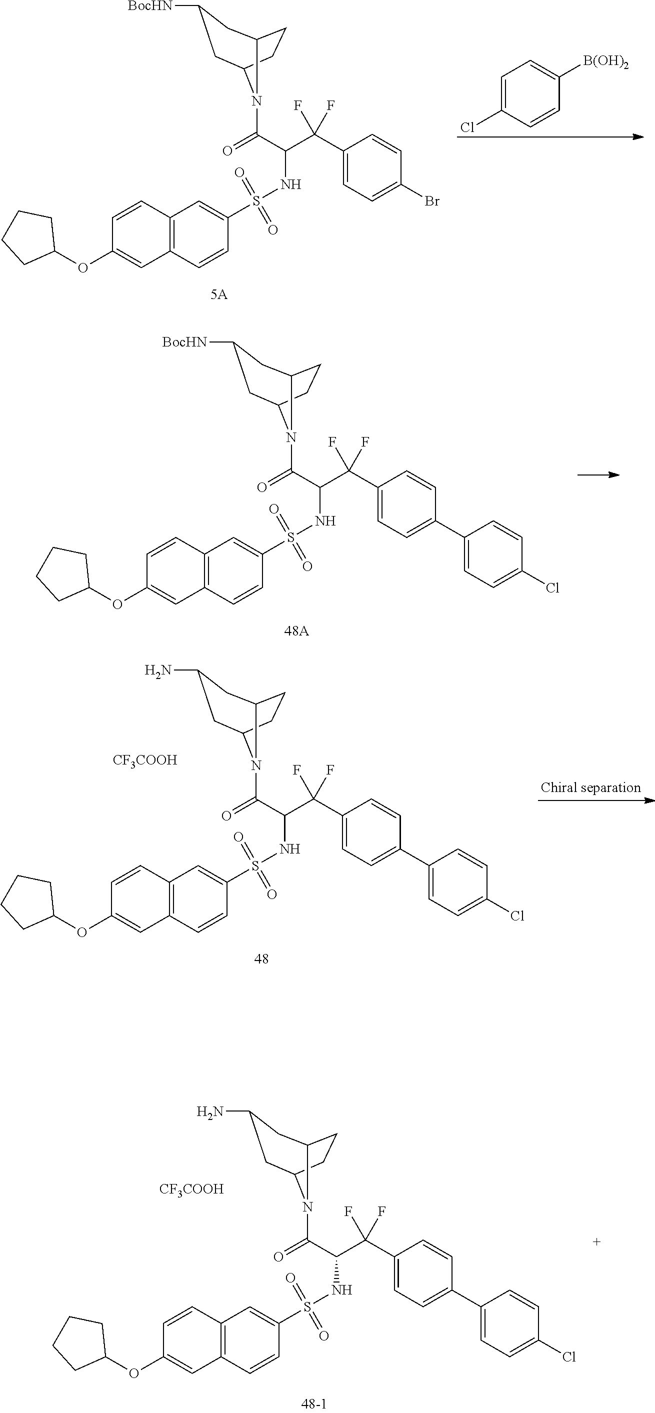

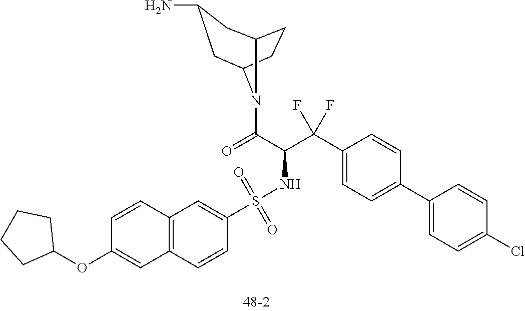 [SMILES: NC1CC2CCC(C1)N2C(=O)[C@H](NS(=O)(=O)c1ccc2cc(OC3CCCC3)ccc2c1)C(F)(F)c1ccc(-c2ccc(Cl)cc2)cc1]

Compounds 48A and 48 were synthesized by employing the procedures described for Compounds 14C and 1 using (4-chlorophenyl)boronic acid, Compounds 5A, and 48A in lieu of Compounds 14B, 14A, and 1A.

Compound 48A: LC-MS (ESI) m/z: 816 [M+Na]⁺; ¹H-NMR (CDCl₃, 400 MHz): δ (ppm) 0.43-0.52 (m, 1H), 0.78-0.87 (m, 1H), 1.24-1.28 (m, 2H), 1.38-1.46 (m, 9H), 1.57-2.00 (m, 12H), 3.47-3.50 (m, 1H), 3.75-3.87 (m, 1H), 4.11-4.17 (m, 1H), 4.42-4.61 (m, 2H), 4.85-4.88 (m, 1H), 5.96-6.09 (m, 1H), 7.02-7.06 (m, 1H), 7.16-7.26 (m, 1H), 7.40-7.53 (m, 8H), 7.63-7.78 (m, 3H), 8.16-8.21 (m, 1H).

Compound 48, which was separated with chiral HPLC to give Compound 48-1 and Compound 48-2.

Compound 48-1: LC-MS (ESI) m/z: 694 [M+H]⁺; ¹H-NMR (CD₃OD, 400 MHz): δ (ppm) 1.33-2.24 (m, 16H), 3.57-3.79 (m, 1H), 4.51-4.79 (m, 3H), 4.90-4.96 (m, 1H), 6.91-7.22 (m, 3H), 7.32-7.81 (m, 10H), 8.03-8.12 (m, 1H). Chiral separation condition: MeOH contained 0.1% NH₄OH; OD-H (4.6×250 mm, 5 μm); retention time: 4.06 minute.

Compound 48-2: LC-MS (ESI) m/z: 694 [M+H]⁺; ¹H-NMR (CD₃OD, 400 MHz): δ (ppm) 1.31-2.24 (m, 16H), 3.56-3.79 (m, 1H), 4.52-4.78 (m, 3H), 4.88-4.97 (m, 1H), 6.91-7.22 (m, 3H), 7.33-7.80 (m, 10H), 8.02-8.12 (m, 1H). Chiral separation condition: MeOH contained 0.1% NH₄OH; OD-H (4.6×250 mm, 5 μm); retention time: 3.37 minute.

### Example 49

### Synthesis of N-((2R)-1-([1,1′-biphenyl]-4-yl)-3-(3-amino-8-azabicyclo[3.2.1]octan-8-yl)-1,1-difluoro-3-oxopropan-2-yl)-6-(cyclopentyloxy)-N-methylnaphthalene-2-sulfonamide (Compound 49-1), and N-((2S)-1-([1,1′-biphenyl]-4-yl)-3-(3-amino-8-azabicyclo[3.2.1]octan-8-yl)-1,1-difluoro-3-oxopropan-2-yl)-6-(cyclopentyloxy)-N-methylnaphthalene-2-sulfonamide (Compound 49-2)

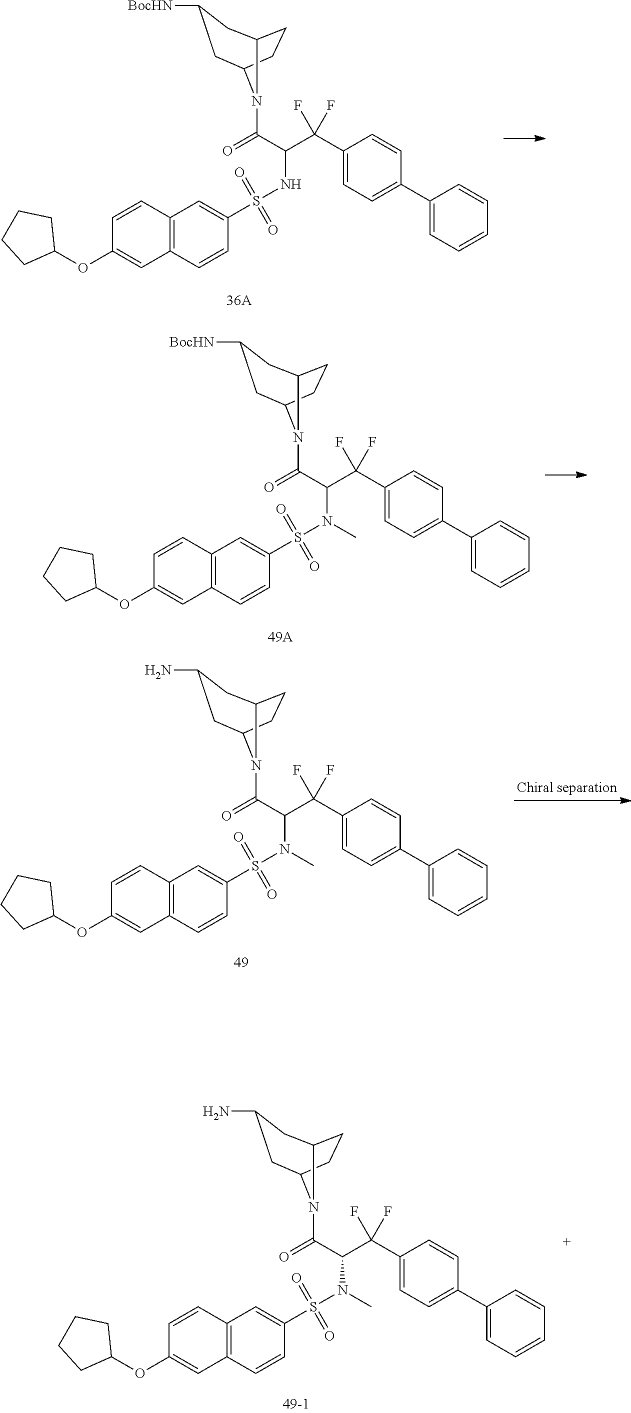 [SMILES: CC(C)(C)OC(=O)NC1CC2CCC(C1)N2C(=O)C(NS(=O)(=O)c1ccc2cc(OC3CCCC3)ccc2c1)C(F)(F)c1ccc(-c2ccccc2)cc1 ; CN(C(C(=O)N1C2CCC1CC(NC(=O)OC(C)(C)C)C2)C(F)(F)c1ccc(-c2ccccc2)cc1)S(=O)(=O)c1ccc2cc(OC3CCCC3)ccc2c1 ; CN(C(C(=O)N1C2CCC1CC(N)C2)C(F)(F)c1ccc(-c2ccccc2)cc1)S(=O)(=O)c1ccc2cc(OC3CCCC3)ccc2c1 ; CN([C@H](C(=O)N1C2CCC1CC(N)C2)C(F)(F)c1ccc(-c2ccccc2)cc1)S(=O)(=O)c1ccc2cc(OC3CCCC3)ccc2c1 ; "+"]

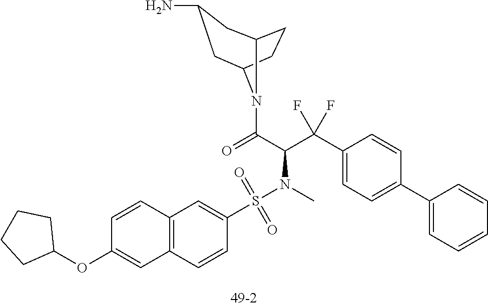 [SMILES: CN([C@@H](C(=O)N1C2CCC1CC(N)C2)C(F)(F)c1ccc(-c2ccccc2)cc1)S(=O)(=O)c1ccc2cc(OC3CCCC3)ccc2c1]

Compounds 49A and 49 were synthesized by employing the procedures described for Compounds 34A and 1 using Compounds 36A and 49A in lieu of Compounds 5A and 1A.

Compound 49A: LC-MS (ESI) m/z: 796 [M+Na]⁺.

Compound 49 was separated with chiral HPLC to furnish Compound 49-1 and Compound 49-2.

Compound 49-1: LC-MS (ESI) m/z: 674 [M+H]⁺; ¹H-NMR (CD₃OD, 400 MHz): δ (ppm) 0.54-1.05 (m, 1H), 1.22-1.30 (m, 1H), 1.52-1.90 (m, 14H), 2.94-3.03 (m, 1H), 3.08-3.23 (m, 3H), 4.19-4.48 (m, 2H), 4.77 (s, 1H), 5.36-5.51 (m, 1H), 7.05-7.08 (m, 2H), 7.28-7.54 (m, 10H), 7.68 (d, J=8.8 Hz, 1H), 7.77 (t, J=8.4 Hz, 1H), 8.13 (d, J=8.8 Hz, 1H). Chiral separation condition: MeOH contained 0.5% NH₄OH; OJ-H (4.6×250 mm, 5 μm); retention time: 4.81 minute.

Compound 49-2: LC-MS (ESI) m/z: 674 [M+H]⁺; ¹H-NMR (CD₃OD, 400 MHz): δ (ppm) 0.55-1.10 (m, 1H), 1.25-1.37 (m, 1H), 1.56-1.94 (m, 14H), 2.96-3.05 (m, 1H), 3.18-3.33 (m, 3H), 4.25-4.56 (m, 2H), 4.84 (s, 1H), 5.47-5.61 (m, 1H), 7.12 (d, J=9.6 Hz, 2H), 7.33-7.62 (m, 10H), 7.74 (d, J=8.8 Hz, 1H), 7.82 (t, J=8.8 Hz, 1H), 8.23 (d, J=8.0 Hz, 1H). Chiral separation condition: MeOH contained 0.5% NH₄OH; OJ-H (4.6×250 mm, 5 μm); retention time: 3.45 minute.

### Example 50

### Synthesis of N-((2R)-3-(3-amino-8-azabicyclo[3.2.1]octan-8-yl)-1-(4′-chloro-[1,1′-biphenyl]-4-yl)-1,1-difluoro-3-oxopropan-2-yl)-4-(cyclohexylmethoxy) benzenesulfonamide trifluoroacetate (Compound 50-1), and N-((2S)-3-(3-amino-8-azabicyclo[3.2.1]octan-8-yl)-1-(4′-chloro-[1,1′-biphenyl]-4-yl)-1,1-difluoro-3-oxopropan-2-yl)-4-(cyclohexylmethoxy)benzenesulfonamide trifluoroacetate (Compound 50-2)

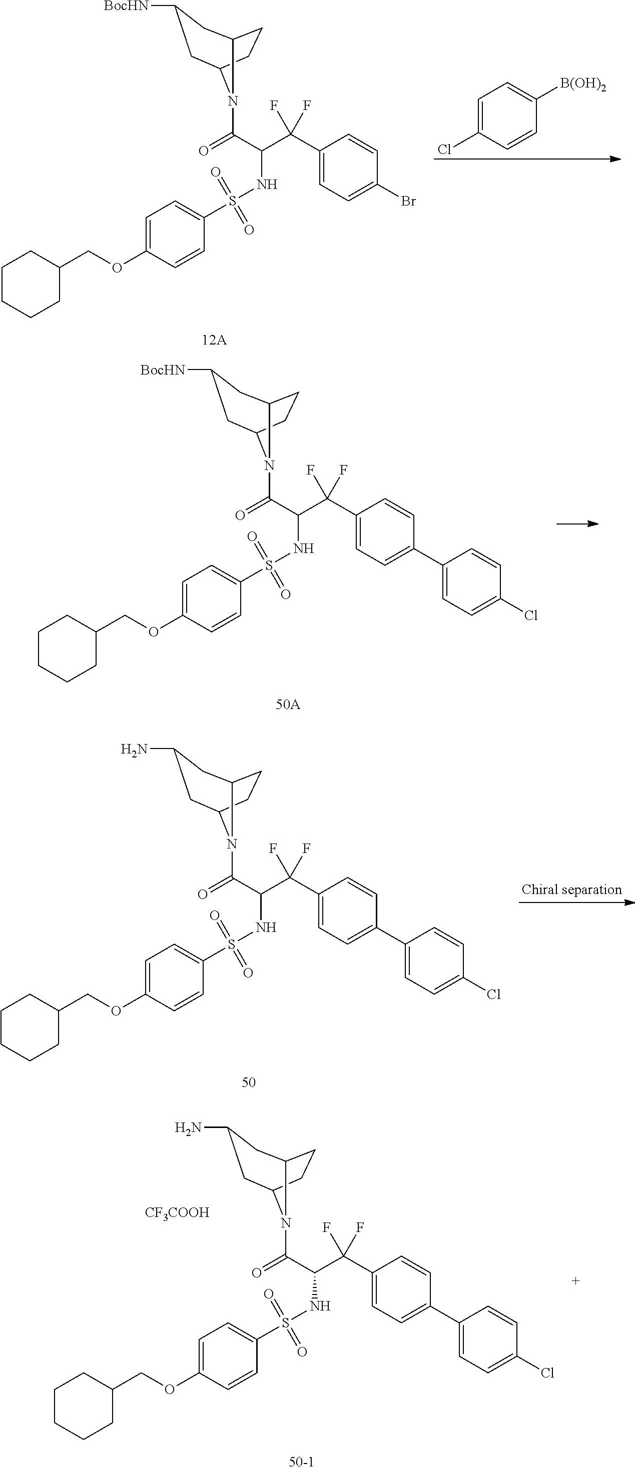

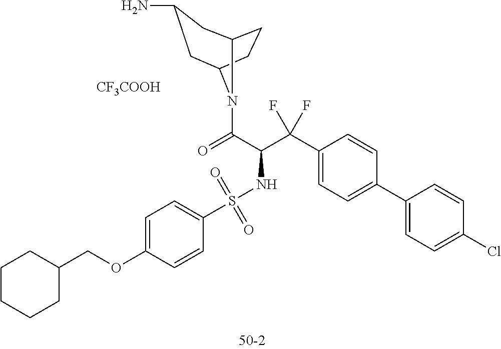 [SMILES: NC1CC2CCC(C1)N2C(=O)[C@H](NS(=O)(=O)c1ccc(OCC2CCCCC2)cc1)C(F)(F)c1ccc(-c2ccc(Cl)cc2)cc1 ; "CF3COOH"]

Compounds 50A and 50 were synthesized by employing the procedures described for Compounds 14C and 6 using (4-chlorophenyl)boronic acid, Compounds 12A, and 50A in lieu of Compounds 14B, 14A, and 6C.

Compound 50A: LC-MS (ESI) m/z: 772 [M+H]⁺.

Compound 50 was separated with chiral HPLC to furnish Compound 50-1 and Compound 50-2.

Compound 50-1: LC-MS (ESI) m/z: 672 [M+H]⁺; ¹H-NMR (CD₃OD, 400 MHz): δ (ppm) 0.82-0.96 (m, 2H), 1.14-1.35 (m, 3H), 1.66-2.18 (m, 14H), 3.39-3.42 (m, 1H), 3.54-3.72 (m, 2H), 4.58-4.82 (m, 3H), 6.67-6.77 (m, 2H), 7.35-7.49 (m, 7H), 7.55-7.63 (m, 3H). Chiral separation condition: n-hexane/EtOH contained 0.1% NH₄OH, 60/40; R,R-Whelk (4.6×250 mm, 5 μm); retention time: 4.69 minute.

Compound 50-2: LC-MS (ESI) m/z: 672 [M+H]⁺; ¹H-NMR (CD₃OD, 400 MHz): δ (ppm) 0.82-0.96 (m, 2H), 1.14-1.35 (m, 3H), 1.66-2.18 (m, 14H), 3.39-3.42 (m, 1H), 3.54-3.72 (m, 2H), 4.58-4.82 (m, 3H), 6.67-6.77 (m, 2H), 7.35-7.49 (m, 7H), 7.55-7.63 (m, 3H). Chiral separation condition: n-hexane/EtOH contained 0.1% NH₄OH, 60/40; R,R-Whelk (4.6×250 mm, 5 μm); retention time: 5.52 minute.

### Example 51

### Synthesis of N-((2R)-3-(3-amino-8-azabicyclo[3.2.1]octan-8-yl)-1-(4-butoxyphenyl)-1,1-difluoro-3-oxopropan-2-yl)-6-(cyclopentyloxy)-N-methylnaphthalene-2-sulfonamide (Compound 51-1), and N-((2S)-3-(3-amino-8-azabicyclo[3.2.1]octan-8-yl)-1-(4-butoxyphenyl)-1,1-difluoro-3-oxopropan-2-yl)-6-(cyclopentyloxy)-N-methylnaphthalene-2-sulfonamide (Compound 51-2)

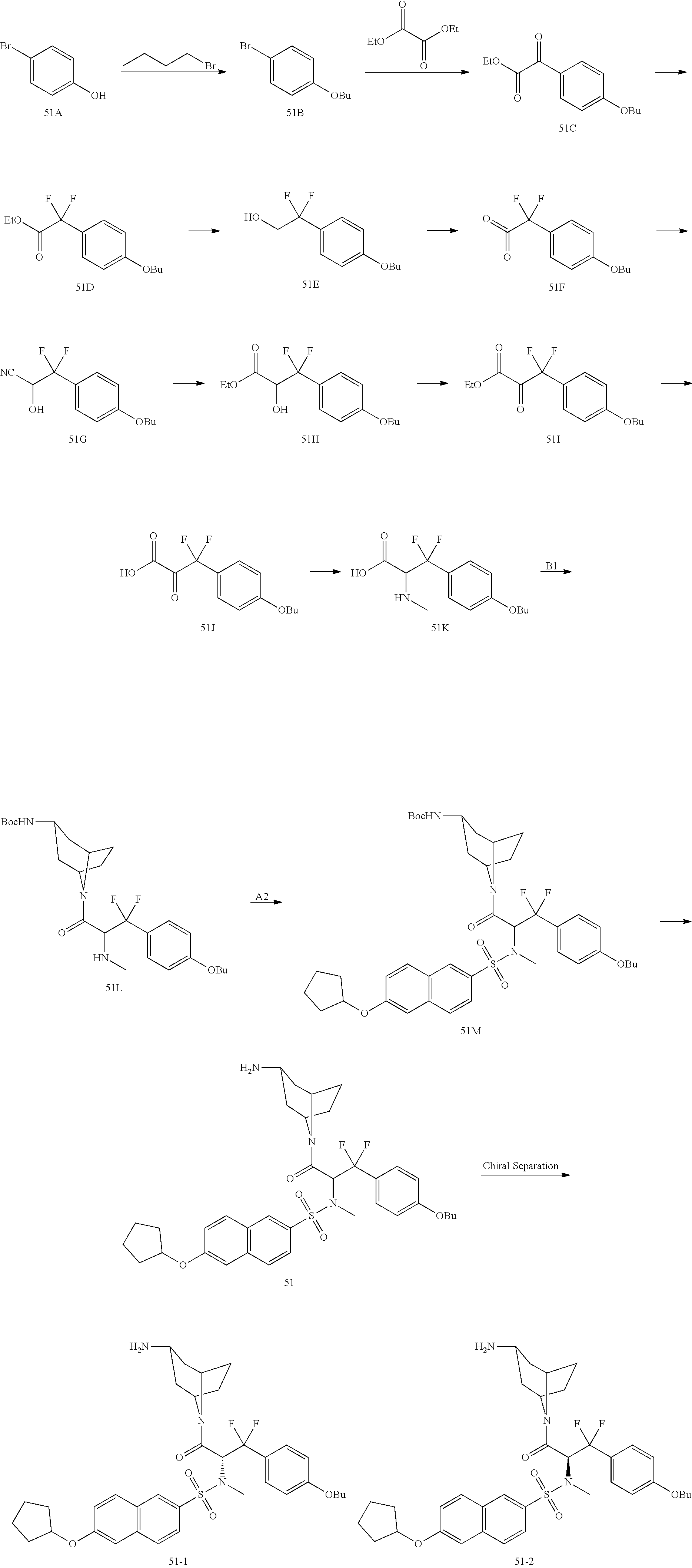

Compounds 51B, 51C, and 51D were synthesized by employing the procedures described for Compounds 4D, 9B, and Compound B2-3 using Compounds 51A using K₂CO₃ as base, 51B, and 51C in lieu of Compounds 4C using Cs₂CO₃ as base, 9A, and Compound B2-2.

Compound 51B: LC-MS (ESI) m/z: No; ¹H-NMR (CDCl₃, 400 MHz): δ (ppm) 0.97 (t, J=7.2 Hz, 3H), 1.43-1.53 (m, 2H), 1.72-1.79 (m, 2H), 3.92 (t, J=6.4 Hz, 2H), 6.77 (d, J=15.6 Hz, 2H), 7.36 (d, J=15.2 Hz, 2H).

Compound 51C: LC-MS (ESI) m/z: 251 [M+H]⁺; ¹H-NMR (CDCl₃, 400 MHz): δ (ppm) 0.99 (t, J=7.2 Hz, 3H), 1.42 (t, J=7.2 Hz, 3H), 1.46-1.55 (m, 2H), 1.73-1.84 (m, 2H), 4.05 (t, J=6.4 Hz, 2H), 4.44 (q, J=7.2 Hz, 2H), 6.97 (d, J=8.8 Hz, 2H), 7.99 (d, J=8.8 Hz, 2H).

Compound 51D: LC-MS (ESI) m/z: 253 [M−F]⁺; ¹H-NMR (CDCl₃, 400 MHz): δ (ppm) 0.98 (t, J=7.6 Hz, 3H), 1.30 (t, J=7.2 Hz, 3H), 1.45-1.54 (m, 2H), 1.74-1.81 (m, 2H), 3.98 (t, J=6.4 Hz, 2H), 4.29 (q, J=7.2 Hz, 2H), 6.93 (d, J=8.8 Hz, 2H), 7.52 (d, J=8.8 Hz, 2H).

To a solution of Compound 51D (9.7 g, 35.6 mmol) in methanol (100 mL) at 0° C. was added NaBH₄ (2.7 g, 71.2 mmol) in several small portions. The reaction mixture was stirred for 1 hour, diluted with water (200 mL), and extracted with dichloromethane (100 mL×2). The combined organic extracts were washed with water (100 mL×2) and brine (200 mL), dried over anhydrous sodium sulfate, filtered, and concentrated. The residue was purified by silica gel column chromatography to afford Compound 51E. LC-MS (ESI) m/z: 211 [M−F]⁺; ¹H-NMR (CDCl₃, 400 MHz): δ (ppm) 0.98 (t, J=7.6 Hz, 3H), 1.45-1.54 (m, 2H), 1.74-1.81 (m, 2H), 1.97-2.00 (m, 1H), 3.91-4.00 (m, 4H), 6.94 (d, J=8.8 Hz, 2H), 7.52 (d, J=8.8 Hz, 2H).

Compounds 51F, 51G, 51H, 51I, 51J, 51K, 51L, 51M, and 51 were synthesized by employing the procedures described for Compound B6-3, Compounds 45F, 45G, Compound B6-3, Compounds 451, 45J, Compound B4, Compounds 1A, and 6 using Compounds 51E, 51F, 51G, 51H, 51I, 51J, 51K, Compounds B1, A2, Compounds 51L, and 51M in lieu of Compound B6-2, Compounds 45E, 45F, Compound B6-2, Compounds 45H, 45I, Compounds B2-6, B4-1, A1, B2, and Compound 6C.

Compound 51F was directly used for the next step without further purification.

Compound 51G: ¹H-NMR (CDCl₃, 400 MHz): δ (ppm) 0.98 (t, J=7.6 Hz, 3H), 1.45-1.55 (m, 2H), 1.75-1.82 (m, 2H), 3.54 (brs, 1H), 4.00 (t, J=6.8 Hz, 2H), 4.78 (t, J=8.4 Hz, 1H), 6.97 (d, J=8.8 Hz, 2H), 7.48 (d, J=8.8 Hz, 2H).

Compound 51H: LC-MS (ESI) m/z: 283 [M−F]⁺; ¹H-NMR (CDCl₃, 400 MHz): δ (ppm) 0.98 (t, J=7.6 Hz, 3H), 1.27 (t, J=6.8 Hz, 3H), 1.44-1.54 (m, 2H), 1.74-1.81 (m, 2H), 3.24 (brs, 1H), 3.98 (t, J=6.4 Hz, 2H), 4.27 (q, J=7.6 Hz, 2H), 4.48-4.53 (m, 1H), 6.92 (d, J=8.8 Hz, 2H), 7.40 (d, J=8.8 Hz, 2H).

Compound 51I: LC-MS (ESI) m/z: non-ionizable compound under routine conditions used; ¹H-NMR (CDCl₃, 400 MHz): δ (ppm) 0.98 (t, J=7.2 Hz, 3H), 1.38 (t, J=7.2 Hz, 3H), 1.44-1.54 (m, 2H), 1.74-1.81 (m, 2H), 3.98 (t, J=6.4 Hz, 2H), 4.38 (q, J=7.2 Hz, 2H), 6.92 (d, J=8.8 Hz, 2H), 7.50 (d, J=8.8 Hz, 2H).

Compound 51J: LC-MS (ESI) m/z: 271 [M−H]⁺; ¹H-NMR (CDCl₃, 400 MHz): δ (ppm) 0.93 (t, J=7.6 Hz, 3H), 1.39-1.48 (m, 2H), 1.66-1.73 (m, 2H), 4.00 (t, J=6.4 Hz, 2H), 7.00 (d, J=8.8 Hz, 2H), 7.51 (d, J=8.8 Hz, 2H).

Compound 51K: LC-MS (ESI) m/z: 286 [M−H]⁺; ¹H-NMR (DMSO-d₆, 400 MHz): δ (ppm) 0.94 (t, J=7.6 Hz, 3H), 1.39-1.49 (m, 2H), 1.66-1.74 (m, 2H), 2.38 (s, 3H), 3.94-4.02 (m, 3H), 6.99 (d, J=8.8 Hz, 2H), 7.41 (d, J=8.8 Hz, 2H).

Compound 51L: LC-MS (ESI) m/z: 496.3 [M+H]⁺; ¹H-NMR (CDCl₃, 400 MHz): δ (ppm) 0.95-0.99 (m, 3H), 1.27-1.34 (m, 2H), 1.43-1.52 (m, 11H), 1.73-2.13 (m, 8H), 2.31-2.36 (m, 3H), 3.73-3.76 (m, 1H), 3.95-4.00 (m, 2H), 4.17-4.32 (m, 2H), 4.74-4.84 (m, 1H), 6.87-6.93 (m, 2H), 7.35-7.43 (m, 2H).

Compound 51M: LC-MS (ESI) m/z: 670 [M−Boc+H]⁺.

Compound 51, which was separated with chiral HPLC to give Compound 51-1 and Compound 51-2.

Compound 51-1: LC-MS (ESI) m/z: 670 [M+H]⁺; ¹H-NMR (CDCl₃, 400 MHz): δ (ppm) 0.28-1.09 (m, 4H), 1.26-1.50 (m, 6H), 1.62-2.05 (m, 15H), 2.95-3.09 (m, 1H), 3.12-3.25 (m, 3H), 3.92-3.96 (m, 2H), 4.13-4.51 (m, 1H), 4.60-4.61 (m, 1H), 4.90-4.95 (m, 1H), 5.35-5.53 (m, 1H), 6.84-6.91 (m, 2H), 7.13-7.14 (m, 1H), 7.19-7.23 (m, 1H), 7.34-7.47 (m, 2H), 7.65-7.68 (m, 1H), 7.75-7.84 (m, 2H), 8.23-8.26 (m, 1H). Chiral separation condition: MeOH contained 0.1% NH₄OH; AD-H (4.6×250 mm, 5 μm); retention time: 4.77 minute.

Compound 51-2: LC-MS (ESI) m/z: 670 [M+H]⁺; ¹H-NMR (CDCl₃, 400 MHz): δ (ppm) 0.28-1.09 (m, 5H), 1.45-2.03 (m, 20H), 2.94-3.43 (m, 4H), 3.93-3.96 (m, 2H), 4.13-4.51 (m, 1H), 4.60-4.61 (m, 1H), 4.90-4.94 (m, 1H), 5.35-5.53 (m, 1H), 6.84-6.91 (m, 2H), 7.13-7.14 (m, 1H), 7.19-7.23 (m, 1H), 7.34-7.48 (m, 2H), 7.65-7.68 (m, 1H), 7.75-7.83 (m, 2H), 8.23-8.26 (m, 1H); Chiral separation condition: MeOH contained 0.1% NH₄OH; AD-H (4.6×250 mm, 5 μm); retention time: 7.33 minute.

### Example 52

### Synthesis of N-((2R)-3-(3-amino-8-azabicyclo[3.2.1]octan-8-yl)-1-(4-chlorophenyl)-1,1-difluoro-3-oxopropan-2-yl)-6-(cyclopentyloxy)-N-methylnaphthalene-2-sulfonamide (Compound 52-1), and N-((2S)-3-(3-amino-8-azabicyclo[3.2.1]octan-8-yl)-1-(4-chlorophenyl)-1,1-difluoro-3-oxopropan-2-yl)-6-(cyclopentyloxy)-N-methylnaphthalene-2-sulfonamide (Compound 52-2)

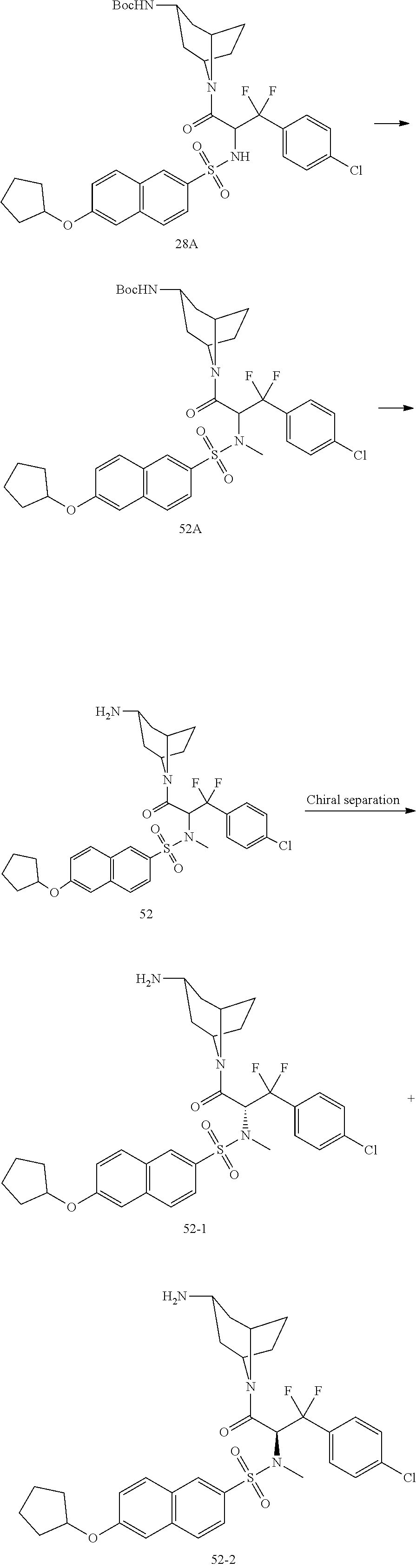 [SMILES: CC(C)(C)OC(=O)NC1CC2CCC(C1)N2C(=O)C(NS(=O)(=O)c1ccc2cc(OC3CCCC3)ccc2c1)C(F)(F)c1ccc(Cl)cc1 ; CN(C(C(=O)N1C2CCC1CC(NC(=O)OC(C)(C)C)C2)C(F)(F)c1ccc(Cl)cc1)S(=O)(=O)c1ccc2cc(OC3CCCC3)ccc2c1 ; CN(C(C(=O)N1C2CCC1CC(N)C2)C(F)(F)c1ccc(Cl)cc1)S(=O)(=O)c1ccc2cc(OC3CCCC3)ccc2c1 ; CN([C@H](C(=O)N1C2CCC1CC(N)C2)C(F)(F)c1ccc(Cl)cc1)S(=O)(=O)c1ccc2cc(OC3CCCC3)ccc2c1 ; CN([C@@H](C(=O)N1C2CCC1CC(N)C2)C(F)(F)c1ccc(Cl)cc1)S(=O)(=O)c1ccc2cc(OC3CCCC3)ccc2c1]

Compounds 52A and 52 were synthesized by employing the procedures described for Compounds 34A and 1 using Compounds 28A and 52A in lieu of Compounds 5A and 1A.

Compound 52A: LC-MS (ESI) m/z: 754 [M+Na]⁺.

Compound 52, which was separated with chiral HPLC (n-hexane/EtOH contained 0.1% NH₄OH, 60/40; AY-H, 250×4.6 mm, 5 μm) to give Compound 52-1 and Compound 52-2.

Compound 52-1: LC-MS (ESI) m/z: 632 [M+H]⁺; ¹H-NMR (CD₃OD, 400 MHz): δ (ppm) 0.76-0.91 (s, 1H), 1.49-2.20 (m, 15H), 3.07-3.68 (m, 4H), 4.34-4.58 (m, 2H), 4.98-5.01 (m, 1H), 5.33-5.58 (m, 1H), 7.22-7.59 (m, 7H), 7.80-7.94 (m, 2H), 8.18-8.26 (m, 1H). Chiral separation condition: n-hexane/EtOH contained 0.1% NH₄OH, 60/40; AY-H (250×4.6 mm, 5 μm); retention time: 18.34 minute.

Compound 52-2: LC-MS (ESI) m/z: 632 [M+H]⁺; ¹H-NMR (CD₃OD, 400 MHz): δ (ppm) 0.74-0.91 (s, 1H), 1.53-2.20 (m, 15H), 3.07-3.64 (m, 4H), 4.33-4.59 (m, 2H), 4.98-5.01 (m, 1H), 5.32-5.62 (m, 1H), 7.22-7.62 (m, 7H), 7.80-7.94 (m, 2H), 8.18-8.26 (m, 1H); Chiral separation condition: n-hexane/EtOH contained 0.1% NH₄OH, 60/40; AY-H (250×4.6 mm, 5 μm); retention time: 7.60 minute.

### Example 53

### Synthesis of (R)—N-(3-(4-aminopiperidin-1-yl)-1-(4-butoxyphenyl)-1,1-difluoro-3-oxopropan-2-yl)-6-(cyclopentyloxy)-N-methylnaphthalene-2-sulfonamide (Compound 53-1), and (S)—N-(3-(4-aminopiperidin-1-yl)-1-(4-butoxyphenyl)-1,1-difluoro-3-oxopropan-2-yl)-6-(cyclopentyloxy)-N-methylnaphthalene-2-sulfonamide (Compound 53-2)

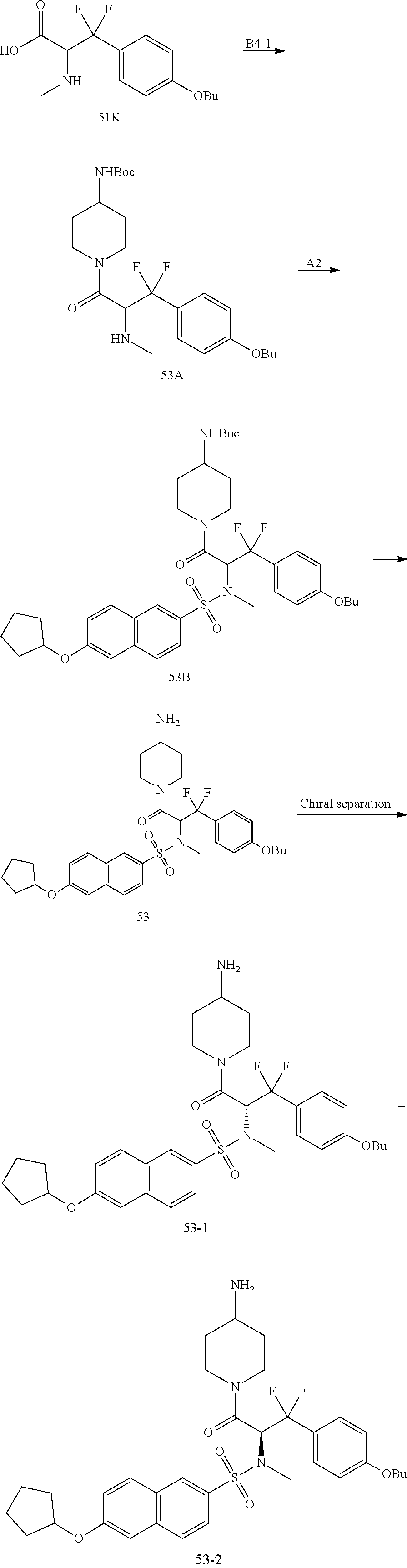

Compounds 53A, 53B, and 53 were synthesized by employing the procedures described for Compound B4, Compounds 1A, and 1 using Compounds 51K, 53A, Compound A2, and Compound 53B in lieu of Compounds B2-6, B2, A1, and Compound 1A.

Compound 53A: LC-MS (ESI) m/z: 470 [M+H]⁺; ¹H-NMR (CDCl₃, 400 MHz): δ (ppm) 0.97 (t, J=7.6 Hz, 3H), 1.48-1.54 (m, 11H), 1.74-1.81 (m, 4H), 1.96-2.04 (m, 4H), 2.29 (d, J=10.8 Hz, 3H), 3.60-3.82 (m, 1H), 3.82-4.01 (m, 4H), 4.42-4.85 (m, 2H), 6.87-6.91 (m, 2H), 7.34-7.38 (m, 2H).

Compound 53B: LC-MS (ESI) m/z: 644 [M−Boc+H]⁺.

Compound 53, which was separated with chiral HPLC to give Compound 53-1 and Compound 53-2.

Compound 53-1: LC-MS (ESI) m/z: 644 [M+H]⁺; ¹H-NMR (CDCl₃, 400 MHz): δ (ppm) 0.67-0.76 (m, 1H), 0.98 (t, J=7.6 Hz, 3H), 1.25-1.54 (m, 6H), 1.75-2.03 (m, 10H), 2.53-2.63 (m, 1H), 2.82-2.91 (m, 1H), 3.13 (d, J=7.6 Hz, 3H), 3.92-4.02 (m, 3H), 4.27-4.38 (m, 1H), 4.90-4.93 (m, 1H), 5.63-5.72 (m, 1H), 6.83-6.89 (m, 2H), 7.13 (s, 1H), 7.20 (d, J=11.2 Hz, 1H), 7.34-7.41 (m, 2H), 7.63-7.66 (m, 1H), 7.74-7.82 (m, 2H), 8.22 (m, 1H); Chiral separation condition: MeOH contained 0.5% NH₄OH; IC (4.6×250 mm, 5 μm); retention time: 4.88 minute.

Compound 53-2: LC-MS (ESI) m/z: 644 [M+H]⁺; ¹H-NMR (CDCl₃, 400 MHz): δ (ppm) 0.67-0.76 (m, 1H), 0.98 (t, J=7.6 Hz, 3H), 1.44-2.05 (m, 16H), 2.53-2.63 (m, 1H), 2.78-2.90 (m, 1H), 3.10 (d, J=7.2 Hz, 3H), 3.93-4.01 (m, 3H), 4.27-4.36 (m, 1H), 4.90-4.94 (m, 1H), 5.63-5.71 (m, 1H), 6.83-6.89 (m, 2H), 7.14 (s, 1H), 7.19-7.22 (m, 1H), 7.35-7.41 (m, 2H), 7.63-7.66 (m, 1H), 7.75-7.82 (m, 2H), 8.22 (m, 1H); Chiral separation condition: MeOH contained 0.5% NH₄OH; IC (4.6×250 mm, 5 μm); retention time: 6.16 minute.

### Example 54

### Synthesis of N-((2R)-3-(3-amino-8-azabicyclo[3.2.1]octan-8-yl)-1,1-difluoro-3-oxo-1-(4-(trifluoromethyl)phenyl)propan-2-yl)-6-(cyclopentyloxy)naphthalene-2-sulfonamide (Compound 54-1), and N-((2S)-3-(3-amino-8-azabicyclo[3.2.1]octan-8-yl)-1,1-difluoro-3-oxo-1-(4-(trifluoromethyl)phenyl)propan-2-yl)-6-(cyclopentyloxy) naphthalene-2-sulfonamide (Compound 54-2)

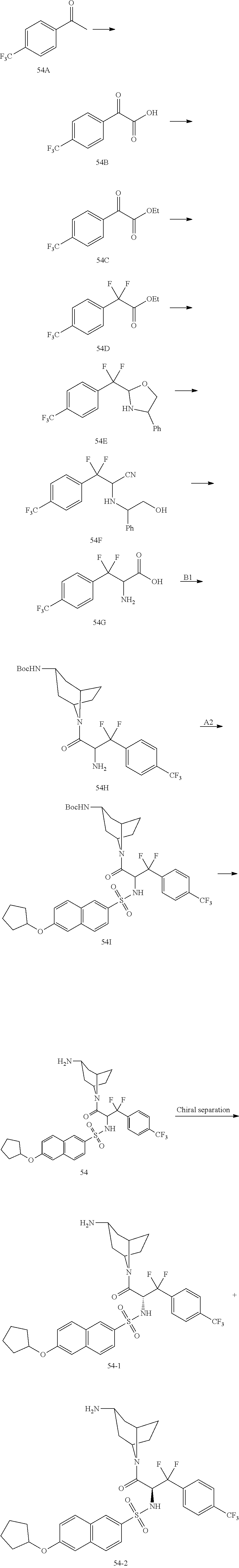

Compounds 54B, 54C, 54D, 54E, 54F, 54G, 54H, 54I, and 54 were synthesized by employing the procedures described for Compounds 42B, 42C, Compounds B2-3, B2-4, B2-5, B2-6, B4, Compounds 1A, and 1 using Compounds 54A, 54B, 54C, 54D, 54E, 54F, 54G, Compounds B1, A2, Compounds 54H, and 54I in lieu of Compounds 42A, 42B, Compounds B2-2, B2-3, B2-4, B2-5, B2-6, B4-1, A1, B2, and Compound 1A.

Compound 54B: LC-MS (ESI) m/z: 217 [M−H]⁺; ¹H-NMR (CDCl₃, 400 MHz): δ (ppm) 7.80 (d, J=8.0 Hz, 2H), 8.41-8.43 (m, 2H).

Compound 54C: LC-MS (ESI) m/z: No; ¹H-NMR (CDCl₃, 400 MHz): δ (ppm) 1.44 (t, J=7.2 Hz, 3H), 4.47 (q, J=7.2 Hz, 2H), 7.78 (d, J=8.8 Hz, 2H), 8.17 (d, J=8.8 Hz, 2H).

Compound 54D: ¹H-NMR (CDCl₃, 400 MHz): δ (ppm) 1.24 (t, J=7.2 Hz, 3H), 4.24 (q, J=7.2 Hz, 2H), 7.64-7.70 (m, 4H).

Compound 54E: LC-MS (ESI) m/z: 344 [M+H]⁺; ¹H-NMR (CDCl₃, 400 MHz): δ (ppm) 2.56-2.66 (m, 1H), 3.34-3.67 (m, 1H), 3.95-4.50 (m, 2H), 5.13-5.26 (m, 1H), 7.18-7.36 (m, 5H), 7.70-7.77 (m, 4H).

Compound 54F: LC-MS (ESI) m/z: 371 [M+H]⁺; ¹H-NMR (CDCl₃, 400 MHz): δ (ppm) 1.73-1.78 (m, 1H), 2.29-2.68 (m, 1H), 3.44-3.66 (m, 1H), 3.71-4.36 (m, 3H), 6.93-7.37 (m, 5H), 7.59-7.77 (m, 4H).

Compound 54G: LC-MS (ESI) m/z: 270 [M+H]⁺; ¹H-NMR (DMSO-d₆, 400 MHz): δ (ppm) 4.20-4.27 (m, 1H), 7.76 (d, J=8.4 Hz, 2H), 7.86 (d, J=8.4 Hz, 2H).

Compound 54H: LC-MS (ESI) m/z: 478 [M+H]⁺; ¹H-NMR (CDCl₃, 400 MHz): δ (ppm) 1.35-1.61 (m, 13H), 1.86-2.07 (m, 4H), 3.93-4.03 (m, 1H), 4.08-4.16 (m, 2H), 4.26-4.34 (m, 1H), 4.63-4.79 (m, 1H), 7.57-7.73 (m, 4H).

Compound 541: LC-MS (ESI) m/z: 652 [M−Boc+H]⁺.

Compound 54 was separated with chiral HPLC to give Compound 54-1 and Compound.

Compound 54-1: LC-MS (ESI) m/z: 652 [M+H]⁺; ¹H-NMR (CDCl₃, 400 MHz): δ (ppm) 1.24-1.30 (m, 2H), 1.55-1.67 (m, 10H), 1.82-2.00 (m, 5H), 2.76-3.04 (m, 1H), 4.07-4.10 (m, 1H), 4.38-4.55 (m, 2H), 4.87-4.92 (m, 1H), 7.10-7.12 (m, 1H), 7.19-7.24 (m, 1H), 7.54-7.59 (m, 2H), 7.64-7.71 (m, 3H), 7.74-7.78 (m, 1H), 7.80-7.82 (m, 1H), 8.18-8.25 (m, 1H); Chiral separation condition: MeOH contained 0.1% NH₄OH; IC (4.6×250 mm, 5 μm); Retention time: 3.42 minute.

Compound 54-2: LC-MS (ESI) m/z: 652 [M+H]⁺; ¹H-NMR (CDCl₃, 400 MHz): δ (ppm) 0.76-1.30 (m, 3H), 1.42-2.01 (m, 12H), 2.08-2.26 (m, 1H), 2.92-3.02 (m, 1H), 3.46-3.69 (m, 1H), 4.17-4.34 (m, 1H), 4.41-4.62 (m, 1H), 4.65-4.87 (m, 2H), 7.00-7.04 (m, 1H), 7.09-7.16 (m, 1H), 7.20-7.25 (m, 3H), 7.40-7.57 (m, 3H), 7.63-7.67 (m, 1H), 8.03-8.07 (m, 1H); Chiral separation condition: MeOH contained 0.1% NH₄OH; IC (4.6×250 mm, 5 μm); Retention time: 2.66 minute.

### Example 55

### Synthesis of N-((2R)-3-(3-amino-8-azabicyclo[3.2.1]octan-8-yl)-1,1-difluoro-3-oxo-1-(4′-(trifluoromethyl)-[1,1′-biphenyl]-4-yl)propan-2-yl)-6-(cyclopentyloxy) naphthalene-2-sulfonamide trifluoroacetate (Compound 55-1), and N-((2S)-3-(3-amino-8-azabicyclo[3.2.1]octan-8-yl)-1,1-difluoro-3-oxo-1-(4′-(trifluoromethyl)-[1,1′-biphenyl]-4-yl)propan-2-yl)-6-(cyclopentyloxy)naphthalene-2-sulfonamide trifluoroacetate (Compound 55-2)

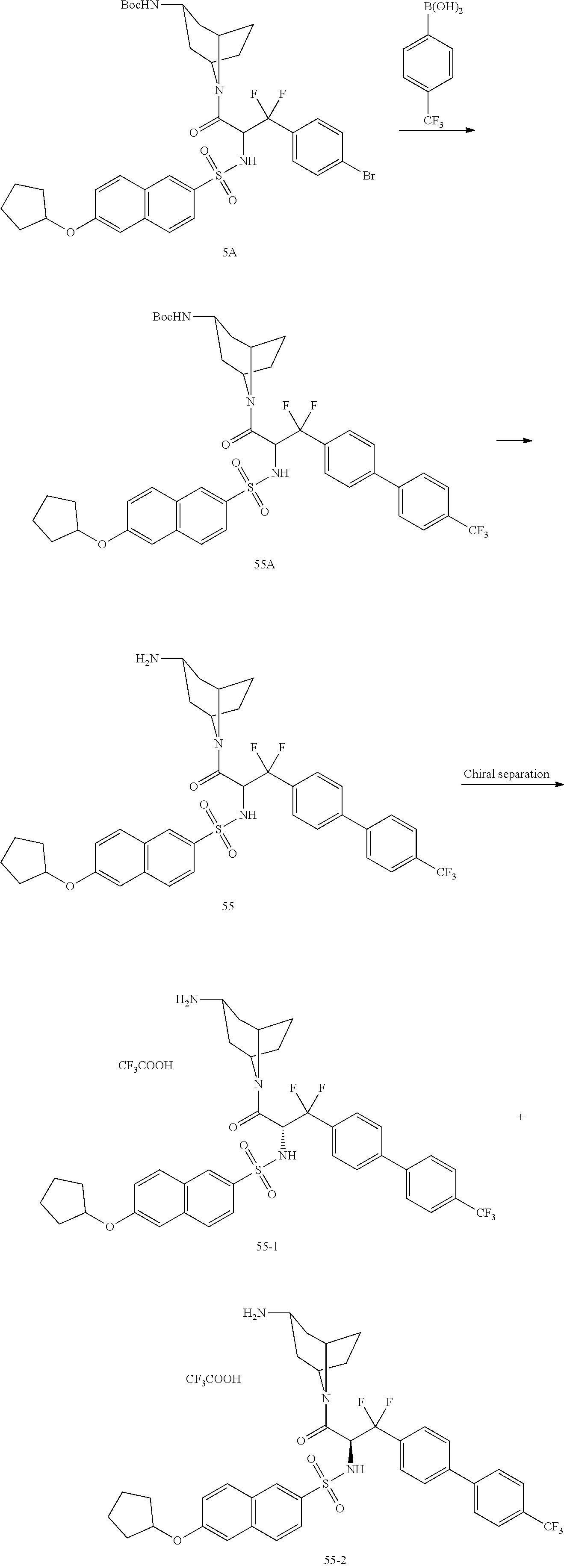

Compounds 55A and 55 were synthesized by employing the procedures described for Compounds 14C and 1 using 4-(trifluoromethyl)phenylboronic acid, Compounds 5A, and 55A in lieu of Compounds 14B, 14A, and 1A.

Compound 55A: LC-MS (ESI) m/z: 850 [M+Na]⁺; ¹H-NMR (CDCl₃, 400 MHz): δ (ppm) 0.43-0.49 (m, 1H), 0.77-0.88 (m, 1H), 1.25-1.30 (m, 2H), 1.59 (s, 9H), 1.67-2.10 (m, 12H), 3.45-3.48 (m, 1H), 4.09-4.18 (m, 1H), 4.42-4.61 (m, 2H), 4.87 (s, 1H), 5.99-6.13 (m, 1H), 7.04-7.07 (m, 1H), 7.17-7.23 (m, 1H), 7.46-7.80 (m, 11H), 8.16-8.21 (m, 1H).

Compound 55 was separated with chiral HPLC to give Compound 55-1 and Compound.

Compound 55-1: LC-MS (ESI) m/z: 728 [M+H]⁺; ¹H-NMR (CD₃OD, 400 MHz): δ (ppm) 1.45-2.09 (m, 16H), 3.56-3.79 (m, 1H), 4.49-4.82 (m, 3H), 4.93-4.99 (m, 1H), 6.94-7.16 (m, 2H), 7.29 (d, J=8.4 Hz, 1H), 7.39 (d, J=12.8 Hz, 1H), 7.49-7.80 (m, 9H), 8.03-8.14 (m, 1H). Chiral separation condition: n-hexane/EtOH contained 0.1% NH₄OH, 70/30; AD-H (250×4.6 mm, 5 μm); retention time: 14.81 minute.

Compound 55-2: LC-MS (ESI) m/z: 728 [M+H]⁺; ¹H-NMR (CD₃OD, 400 MHz): δ (ppm) 1.31-2.23 (m, 16H), 3.56-3.79 (m, 1H), 4.48-4.79 (m, 3H), 4.94-4.99 (m, 1H), 6.96-7.30 (m, 2H), 7.37-7.82 (m, 11H), 8.02-8.13 (m, 1H). Chiral separation condition: n-hexane/EtOH contained 0.1% NH₄OH, 70/30; AD-H (250×4.6 mm, 5 μm); retention time: 21.63 minute.

### Example 56

### Synthesis of N-((2R)-3-(3-amino-8-azabicyclo[3.2.1]octan-8-yl)-1,1-difluoro-1-(4′-fluoro-[1,1′-biphenyl]-4-yl)-3-oxopropan-2-yl)-6-(cyclopentyloxy)naphthalene-2-sulfonamide trifluoroacetate (Compound 56-1), and N-((2S)-3-(3-amino-8-azabicyclo[3.2.1]octan-8-yl)-1,1-difluoro-1-(4′-fluoro-[1,1′-biphenyl]-4-yl)-3-oxopropan-2-yl)-6-(cyclopentyloxy)naphthalene-2-sulfonamide trifluoroacetate (Compound 56-2)

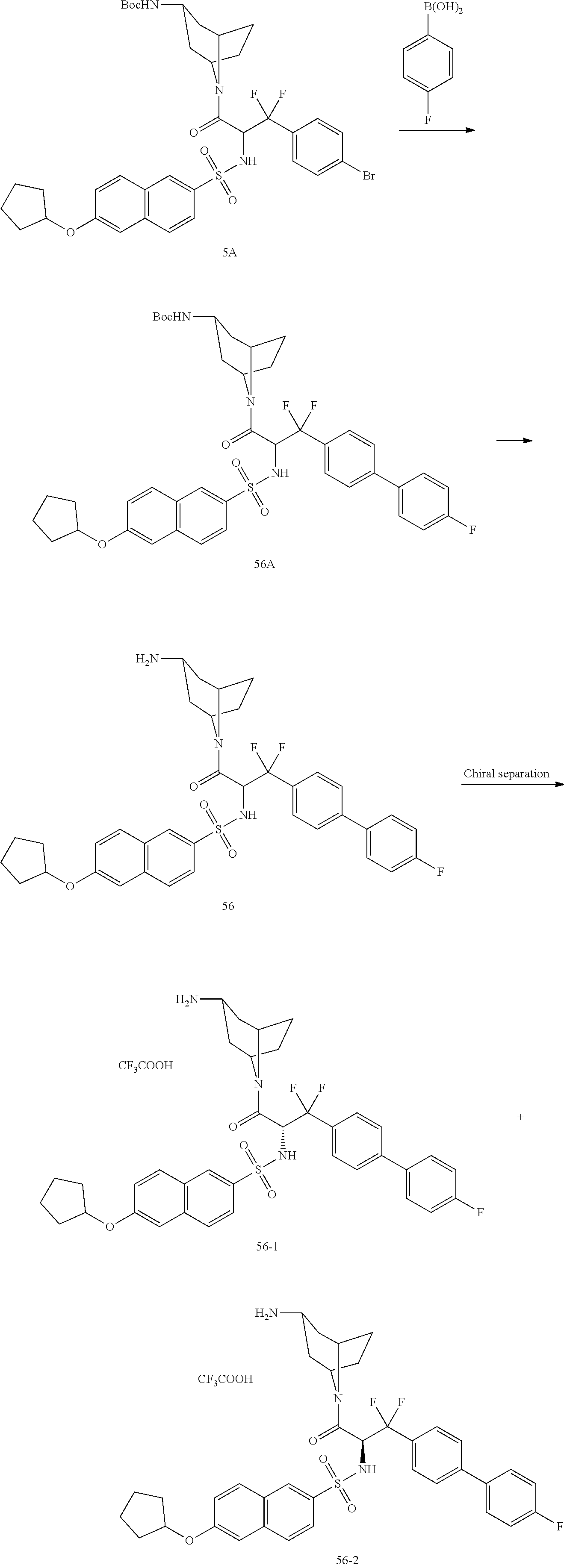

Compounds 56A and 55 were synthesized by employing the procedures described for Compounds 14C and 6 using Compounds 5A and 55A in lieu of Compounds 14A and 6C.

Compound 56A: LC-MS (ESI) m/z: 778 [M+H]⁺.

Compound 56 was separated with chiral HPLC to furnish Compound 56-1 and Compound 56-2.

Compound 56-1: LC-MS (ESI) m/z: 678 [M+H]⁺; ¹H-NMR (CD₃OD, 400 MHz): δ (ppm) 1.014-2.20 (m, 16H), 3.54-3.78 (m, 1H), 4.48-4.75 (m, 3H), 4.91-4.97 (m, 1H), 6.95-7.39 (m, 7H), 7.49-7.82 (m, 6H), 8.04-8.14 (m, 1H). Chiral separation condition: MeOH contained 0.5% NH₄OH; IC (4.6×250 mm, 5 μm); retention time: 8.31 minute.

Compound 56-2: LC-MS (ESI) m/z: 678 [M+H]⁺; ¹H-NMR (CD₃OD, 400 MHz): δ (ppm) 1.01-2.20 (m, 16H), 3.54-3.78 (m, 1H), 4.48-4.75 (m, 3H), 4.91-4.97 (m, 1H), 6.95-7.39 (m, 7H), 7.49-7.82 (m, 6H), 8.04-8.14 (m, 1H). Chiral separation condition: MeOH contained 0.5% NH₄OH; IC (4.6×250 mm, 5 μm); retention time: 6.41 minute.

### Example 57

### Synthesis of (R)—N-(3-(4-(aminomethyl)piperidin-1-yl)-1-(4-bromophenyl)-1,1-difluoro-3-oxopropan-2-yl)-6-(cyclopentyloxy)naphthalene-2-sulfonamide trifluoroacetate (Compound 57-1), and (S)—N-(3-(4-(aminomethyl)piperidin-1-yl)-1-(4-bromophenyl)-1,1-difluoro-3-oxopropan-2-yl)-6-(cyclopentyloxy)naphthalene-2-sulfonamide trifluoroacetate (Compound 57-2)

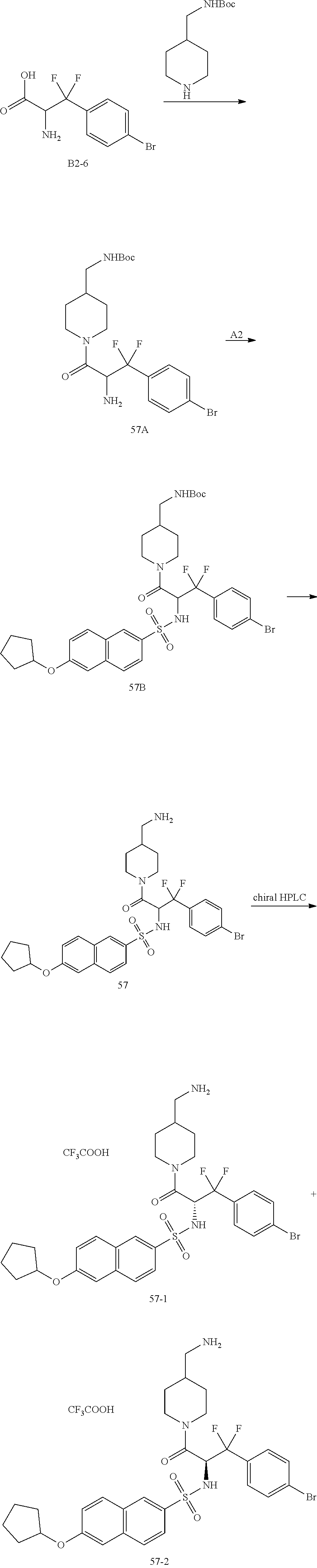

Compounds 57A, 57B, and 57 were synthesized by employing the procedures described for Compound B4, Compounds 1A, and 1 using tert-butyl (piperidin-4-ylmethyl)carbamate, Compound A2, Compounds 57A, and 57B in lieu of Compounds B4-1, A1, B2, and Compound 1A.

Compound 57A: LC-MS (ESI) m/z: 476 [M+H]⁺.

Compound 57B: LC-MS (ESI) m/z: 694 [M−55]⁺.

Compound 57 was separated with chiral HPLC to afford Compound 57-1 and Compound 57-2

Compound 57-1: LC-MS (ESI) m/z: 650 [M+H]⁺; ¹H-NMR (CD₃OD, 400 MHz): δ (ppm) −0.03-0.72 (m, 2H), 1.30-1.76 (m, 9H), 1.90-1.94 (m, 2H), 2.18-2.30 (m, 2H), 2.50-2.68 (m, 2H), 3.77-4.10 (m, 2H), 4.75-4.78 (m, 1H), 4.84-4.88 (m, 1H), 7.06-7.23 (m, 4H), 7.35 (t, J=8.4 Hz, 2H), 7.46 (t, J=10.0 Hz, 1H), 7.64-7.67 (m, 1H), 7.73 (d, J=8.8 Hz, 1H), 8.05 (s, 1H). Chiral separation condition: EtOH contained 0.1% NH₄OH; Cellulose-3 (4.6×250 mm, 5 μm); retention time: 14.15 minute.

Compound 57-2: LC-MS (ESI) m/z: 650 [M+H]⁺; ¹H-NMR (CD₃OD, 400 MHz): δ (ppm) −0.03-0.70 (m, 2H), 1.31-1.77 (m, 9H), 1.91 (s, 2H), 2.19-2.31 (m, 2H), 2.53-2.68 (m, 2H), 3.78-3.82 (m, 1H), 3.88-4.12 (m, 1H), 4.76-4.79 (m, 1H), 4.87 (s, 1H), 7.07-7.24 (m, 4H), 7.36 (t, J=8.0 Hz, 2H), 7.47 (t, J=10.4 Hz, 1H), 7.65-7.68 (m, 1H), 7.74 (d, J=9.2 Hz, 1H), 8.06 (s, 1H). Chiral separation condition: EtOH contained 0.1% NH₄OH; Cellulose-3 (4.6×250 mm, 5 μm); retention time: 7.31 minute.

### Example 58

### Synthesis of (R)—N-(3-(4-aminopiperidin-1-yl)-1-(4-butoxyphenyl)-1,1-difluoro-3-oxopropan-2-yl)-4-(cyclohexylmethoxy)-N-methylbenzenesulfonamide (Compound 58-1), and (S)—N-(3-(4-aminopiperidin-1-yl)-1-(4-butoxyphenyl)-1,1-difluoro-3-oxopropan-2-yl)-4-(cyclohexylmethoxy)-N-methylbenzenesulfonamide (Compound 58-2)

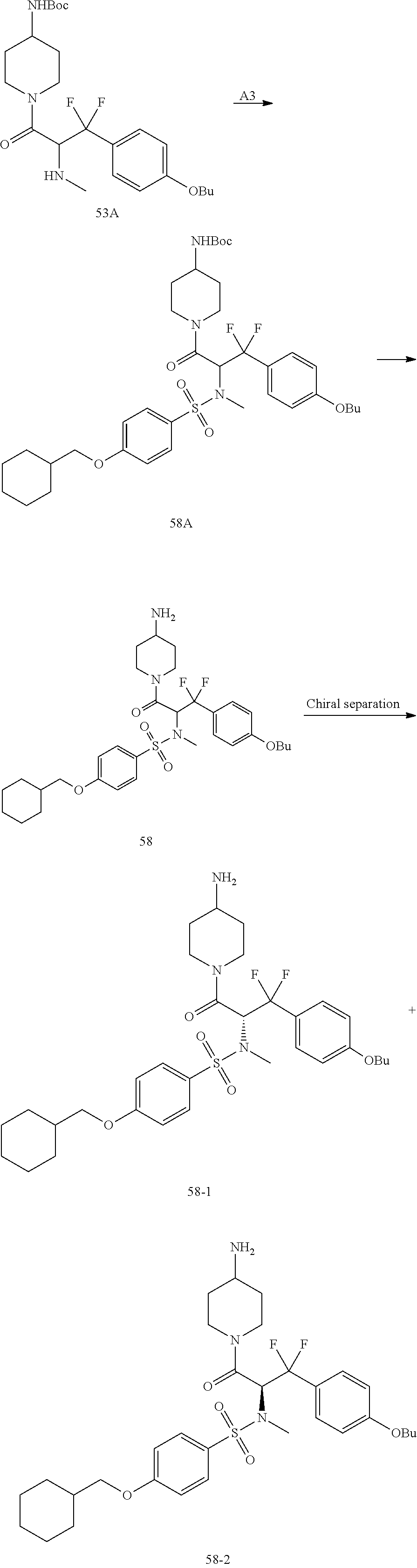

Compounds 58A and 58 were synthesized by employing the procedures described for Compounds 1A and 6 using Compounds 53A, Compound A3, and 58A in lieu of Compounds B2, A1, and Compound 6C.

Compound 58A: LC-MS (ESI) m/z: 622 [M−Boc+H]⁺.

Compound 58 was separated with chiral HPLC to give Compound 58-1 and Compound 58-2.

Compound 58-1: LC-MS (ESI) m/z: 622 [M+H]⁺; ¹H-NMR (CD₃OD, 400 MHz): δ (ppm) 0.89 (t, J=7.2 Hz, 3H), 0.95-1.46 (m, 9H), 1.60-1.78 (m, 10H), 2.47-2.55 (m, 1H), 2.70-3.10 (m, 5H), 3.73-3.75 (m, 2H), 3.88-3.92 (m, 3H), 4.16-4.23 (m, 1H), 5.46-5.56 (m, 1H), 6.80-6.91 (m, 4H), 7.22-7.30 (m, 2H), 7.44-7.49 (m, 2H); Chiral separation condition: MeOH contained 0.5% NH₄OH; (R, R)-Whelk-O1 (4.6×250 mm, 5 μm); retention time: 5.01 minute.

Compound 58-2: LC-MS (ESI) m/z: 622 [M+H]⁺; ¹H-NMR (CD₃OD, 400 MHz): δ (ppm) 0.91 (t, J=7.2 Hz, 3H), 1.00-1.46 (m, 9H), 1.62-2.05 (m, 10H), 2.52-2.64 (m, 1H), 2.88-3.31 (m, 5H), 3.75-3.78 (m, 2H), 3.89-3.95 (m, 2H), 4.14-4.18 (m, 1H), 4.36-4.49 (m, 1H), 5.52-5.60 (m, 1H), 6.80-6.90 (m, 4H), 7.23-7.48 (m, 4H); Chiral separation condition: MeOH contained 0.5% NH₄OH; (R, R)-Whelk-O1 (4.6×250 mm, 5 μm); retention time: 5.79 minute.

### Example 59

### Synthesis of N-((2R)-3-(3-amino-8-azabicyclo[3.2.1]octan-8-yl)-1,1-difluoro-3-oxo-1-(4-(trifluoromethyl)phenyl)propan-2-yl)-4-(cyclohexylmethoxy)benzenesulfonamide (Compound 59-1), and N-((2S)-3-(3-amino-8-azabicyclo[3.2.1]octan-8-yl)-1,1-difluoro-3-oxo-1-(4-(trifluoromethyl)phenyl)propan-2-yl)-4-(cyclohexylmethoxy) benzenesulfonamide (Compound 59-2)

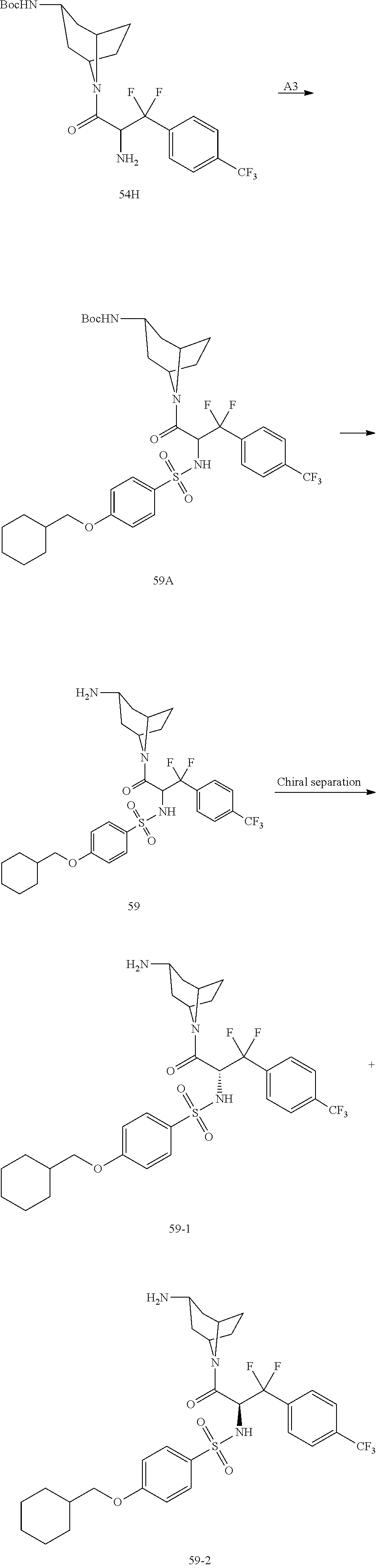 [SMILES: CC(C)(C)OC(=O)NC1CC2CCC(C1)N2C(=O)C(N)C(F)(F)c1ccc(C(F)(F)F)cc1 ; "A3" ; CC(C)(C)OC(=O)NC1CC2CCC(C1)N2C(=O)C(NS(=O)(=O)c1ccc(OCC2CCCCC2)cc1)C(F)(F)c1ccc(C(F)(F)F)cc1 ; NC1CC2CCC(C1)N2C(=O)C(NS(=O)(=O)c1ccc(OCC2CCCCC2)cc1)C(F)(F)c1ccc(C(F)(F)F)cc1 ; NC1CC2CCC(C1)N2C(=O)[C@@H](NS(=O)(=O)c1ccc(OCC2CCCCC2)cc1)C(F)(F)c1ccc(C(F)(F)F)cc1 ; NC1CC2CCC(C1)N2C(=O)[C@H](NS(=O)(=O)c1ccc(OCC2CCCCC2)cc1)C(F)(F)c1ccc(C(F)(F)F)cc1]

Compounds 59A and 59 were synthesized by employing the procedures described for Compounds 1A and 6 using Compounds 54H, Compound A3, and 59A in lieu of Compounds B2, A1, and Compound 6C.

Compound 59A: LC-MS (ESI) m/z: 630 [M−Boc+H]⁺.

Compound 59 was separated with chiral HPLC to give Compound 59-1 and Compound 59-2.

Compound 59-1: LC-MS (ESI) m/z: 630 [M+H]⁺; ¹H-NMR (CD₃OD, 400 MHz): δ (ppm) 0.85-1.39 (m, 8H), 1.58-1.89 (m, 12H), 2.99-3.16 (m, 1H), 3.83 (d, J=6.0 Hz, 2H), 4.29-4.48 (m, 2H), 4.63-4.72 (m, 1H), 6.92-6.99 (m, 2H), 7.61-7.77 (m, 6H). Chiral separation condition: MeOH contained 0.1% NH₄OH; (R, R)-Whelk-O1 (4.6×250 mm, 5 μm); retention time: 4.04 minute.

Compound 59-2: LC-MS (ESI) m/z: 630 [M+H]⁺; ¹H-NMR (CD₃OD, 400 MHz): δ (ppm) 0.75-1.30 (m, 8H), 1.48-1.79 (m, 12H), 2.91-3.04 (m, 1H), 3.73 (d, J=6.0 Hz, 2H), 4.19-4.38 (m, 2H), 4.53-4.62 (m, 1H), 6.82-6.89 (m, 2H), 7.51-7.68 (m, 6H). Chiral separation condition: MeOH contained 0.1% NH₄OH; (R, R)-Whelk-O1 (4.6×250 mm, 5 μm); retention time: 4.62 minute.

### Example 60

### Synthesis of N-((2R)-3-(3-amino-8-azabicyclo[3.2.1]octan-8-yl)-1,1-difluoro-3-oxo-1-(4-(trifluoromethyl)phenyl)propan-2-yl)-4-(cyclopentylmethoxy) benzenesulfonamide (Compound 60-1), and N-((2S)-3-(3-amino-8-azabicyclo[3.2.1]octan-8-yl)-1,1-difluoro-3-oxo-1-(4-(trifluoromethyl)phenyl) propan-2-yl)-4-(cyclopentylmethoxy)benzenesulfonamide (Compound 60-2)

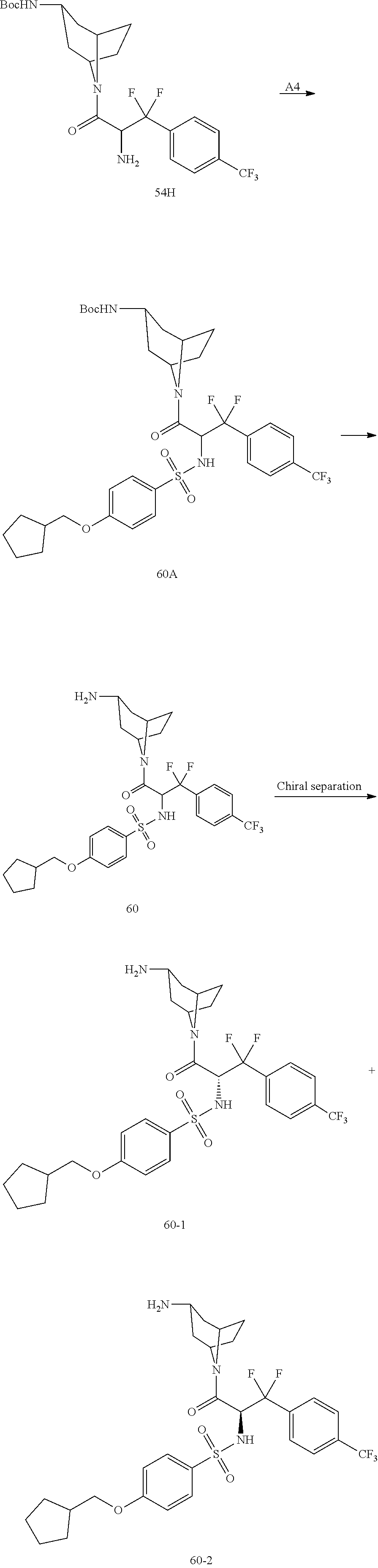 [SMILES: CC(C)(C)OC(=O)NC1CC2CCC(C1)N2C(=O)C(N)C(F)(F)c1ccc(C(F)(F)F)cc1 ; "A4" ; CC(C)(C)OC(=O)NC1CC2CCC(C1)N2C(=O)C(NS(=O)(=O)c1ccc(OCC2CCCC2)cc1)C(F)(F)c1ccc(C(F)(F)F)cc1 ; NC1CC2CCC(C1)N2C(=O)C(NS(=O)(=O)c1ccc(OCC2CCCC2)cc1)C(F)(F)c1ccc(C(F)(F)F)cc1 ; NC1CC2CCC(C1)N2C(=O)[C@@H](NS(=O)(=O)c1ccc(OCC2CCCC2)cc1)C(F)(F)c1ccc(C(F)(F)F)cc1 ; NC1CC2CCC(C1)N2C(=O)[C@H](NS(=O)(=O)c1ccc(OCC2CCCC2)cc1)C(F)(F)c1ccc(C(F)(F)F)cc1]

Compounds 60A and 60 were synthesized by employing the procedures described for Compounds 1A and 6 using Compounds 54H, Compound A4, and 60A in lieu of Compounds B2, A1, and Compound 6C.

Compound 60A: LC-MS (ESI) m/z: 616 [M−Boc+H]⁺.

Compound 60 was separated with chiral HPLC to give Compound 60-1 and Compound 60-2.

Compound 60-1: LC-MS (ESI) m/z: 616 [M+H]⁺; ¹H-NMR (CD₃OD, 400 MHz): δ (ppm) 0.88-1.10 (m, 1H), 1.27-1.45 (m, 4H), 1.60-1.91 (m, 12H), 2.34-2.42 (m, 1H), 3.01-3.17 (m, 1H), 3.90-3.93 (m, 2H), 4.29-4.48 (m, 2H), 4.63-4.72 (m, 1H), 6.93-6.99 (m, 2H), 7.62-7.78 (m, 6H). Chiral separation condition: MeOH contained 0.1% NH₄OH; (R, R)-Whelk-O1 (4.6×250 mm, 5 μm); retention time: 3.11 minute.

Compound 60-2: LC-MS (ESI) m/z: 616 [M+H]⁺; ¹H-NMR (CD₃OD, 400 MHz): δ (ppm) 0.77-0.99 (m, 1H), 1.17-1.34 (m, 4H), 1.51-1.81 (m, 12H), 2.22-2.31 (m, 1H), 2.89-3.04 (m, 1H), 3.80-3.83 (m, 2H), 4.20-4.38 (m, 2H), 4.52-4.62 (m, 1H), 6.83-6.89 (m, 2H), 7.51-7.69 (m, 6H). Chiral separation condition: MeOH contained 0.1% NH₄OH; (R, R)-Whelk-O1 (4.6×250 mm, 5 μm); retention time: 3.53 minute.

### Example 61

### Synthesis of (R)—N-(3-(4-aminopiperidin-1-yl)-1,1-difluoro-3-oxo-1-(4-phenoxyphenyl)propan-2-yl)-6-(cyclopentyloxy)naphthalene-2-sulfonamide (Compound 61-1), and (S)—N-(3-(4-aminopiperidin-1-yl)-1,1-difluoro-3-oxo-1-(4-phenoxyphenyl)propan-2-yl)-6-(cyclopentyloxy)naphthalene-2-sulfonamide (Compound 61-2)

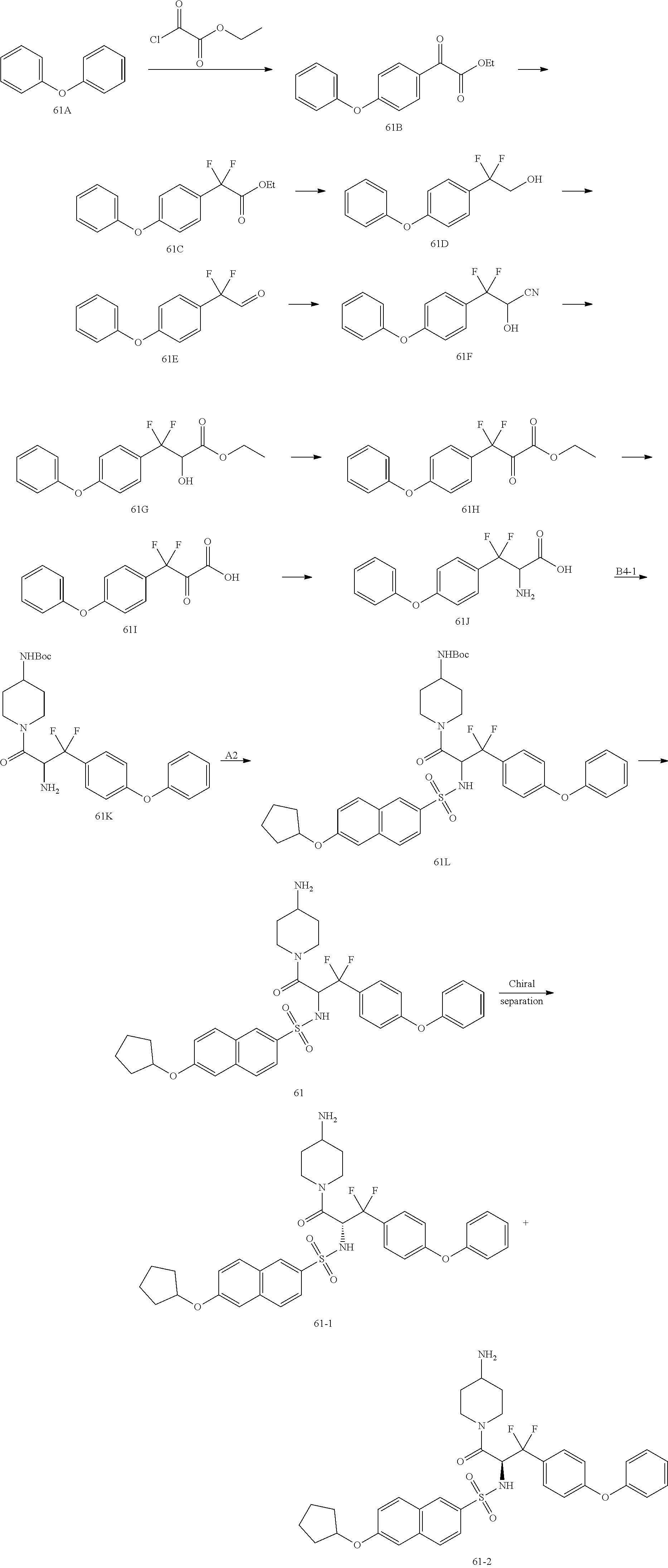

Compounds 61B, 61C, 61D, 61E, 61F, 61G, 61H, 61I [please check 61G, 61H, and 61I as the transformation with respect to using corresponding procedure 45G, B6-3, and 45I do not match], 61J, 61K, 61L, and 61 were synthesized by employing the procedures described for Compounds B2-2, B2-3, Compound 51E, Compound B6-3, Compounds 45F, 45G, Compound B6-3, Compounds 451, 45J, Compound B4, Compounds 2D, and 1 using Compounds 61A, 61B, 61C, 61D, 61E, 61F, 61G, 61H, 61I, 61J, 61K, Compound A2, and Compound 61L in lieu of Compounds B2-1 and B2-2, Compound 51D, Compound B6-2, Compounds 45E, 45F, Compound B6-2, Compounds 45H, 45I, Compounds B4-1, B2, Compounds 2C, and 1A.

Compound 61B: LC-MS (ESI) m/z: 271 [M+H]⁺.

Compound 61C: LC-MS (ESI) m/z: 273 [M−F]⁺.

Compound 61D: LC-MS (ESI) m/z: 231 [M−F]⁺; ¹H-NMR (CD₃OD, 400 MHz): δ (ppm) 3.91-4.00 (m, 2H), 6.72-7.01 (m, 4H), 7.13-7.22 (m, 1H), 7.23-7.37 (m, 3H), 7.46-7.66 (m, 1H).

Compound 61E was used directly for the next step. ¹H-NMR (CDCl₃, 400 MHz): δ (ppm) 6.82-7.07 (m, 4H), 7.11-7.19 (m, 1H), 7.28-7.37 (m, 3H), 7.41-7.56 (m, 1H), 10.23 (s, 1H).

Compound 61F: ¹H-NMR (CDCl₃, 400 MHz): δ (ppm) δ (ppm) 3.64 (s, 1H), 4.80 (t, J=8.0 Hz, 1H), 7.03-7.07 (m, 4H), 7.15-7.46 (m, 3H), 7.48-7.53 (m, 2H).

Compound 61G: LC-MS (ESI) m/z: 323 [M+H]⁺; ¹H-NMR (CDCl₃, 400 MHz): δ (ppm) 1.29 (t, J=7.6 Hz, 3H), 3.24 (d, J=4.0 Hz, 1H), 4.26-4.32 (m, 2H), 4.48-4.55 (m, 1H), 7.00-7.05 (m, 4H), 7.14-7.16 (m, 1H), 7.25-7.27 (m, 2H), 7.33-7.49 (m, 2H).

Compound 61H: ¹H-NMR (CDCl₃, 400 MHz): δ (ppm) 1.28 (t, J=7.6 Hz, 3H), 4.32-4.42 (m, 2H), 6.97-7.06 (m, 4H), 7.12-7.13 (m, 1H), 7.21-7.37 (m, 2H), 7.61-7.58 (m, 2H).

Compound 61I: LC-MS (ESI) m/z: 291 [M−H]⁺; ¹H-NMR (DMSO-d₆, 400 MHz): δ (ppm) 6.99-7.07 (m, 4H), 7.16-7.21 (m, 1H), 7.42-7.62 (m, 4H).

Compound 61J: LC-MS (ESI) m/z: 294 [M+H]⁺; ¹H-NMR (DMSO-d₆, 400 MHz): δ (ppm) 4.21-4.28 (m, 1H), 6.99-7.08 (m, 4H), 7.18-7.21 (m, 1H), 7.38-7.43 (m, 2H), 7.45-7.54 (m, 2H).

Compound 61K: LC-MS (ESI) m/z: 476 [M+H]⁺.

Compound 61L: LC-MS (ESI) m/z: 694 [M−55]⁺.

Compound 61 was separated with chiral HPLC (Chiral separation condition: MeOH contained 0.1% DEA; IC, 250×4.6 mm, 5 m) to give Compound 61-1 and Compound 61-2.

Compound 61-1: LC-MS (ESI) m/z: 650 [M+H]⁺; ¹H-NMR (CD₃OD, 400 MHz): δ (ppm) 0.22-0.58 (m, 1H), 0.82-0.98 (m, 1H), 1.38-2.06 (m, 11H), 2.39-2.81 (m, 3H), 3.45-4.14 (m, 2H), 4.82-5.10 (m, 1H), 6.91-7.04 (m, 4H), 7.17-7.4 (m, 3H), 7.38-7.46 (m, 4H), 7.63-7.70 (m, 1H), 7.82-8.26 (m, 3H). Chiral separation condition: MeOH contained 0.1% NH₄OH; IC (250×4.6 mm, 5 μm); retention time: 5.9 minute.

Compound 61-2: LC-MS (ESI) m/z: 650 [M+H]⁺; ¹H-NMR (CD₃OD, 400 MHz): δ (ppm) 0.22-0.58 (m, 1H), 0.82-0.98 (m, 1H), 1.38-2.06 (m, 11H), 2.39-2.81 (m, 3H), 3.45-4.14 (m, 2H), 4.82-5.10 (m, 1H), 6.91-7.04 (m, 4H), 7.17-7.4 (m, 3H), 7.38-7.46 (m, 4H), 7.63-7.70 (m, 1H), 7.82-8.26 (m, 3H). Chiral separation condition: MeOH contained 0.1% NH₄OH; IC (250×4.6 mm, 5 μm); retention time: 5.1 minute.

### Example 62

### Synthesis of (R)—N-(3-(4-aminopiperidin-1-yl)-1,1-difluoro-3-oxo-1-(4-phenoxy phenyl)propan-2-yl)-6-(cyclopentyloxy)-N-methylnaphthalene-2-sulfonamide trifluoroacetate (Compound 62-1), and (S)—N-(3-(4-aminopiperidin-1-yl)-1,1-difluoro-3-oxo-1-(4-phenoxyphenyl)propan-2-yl)-6-(cyclopentyloxy)-N-methylnaphthalene-2-sulfonamide trifluoroacetate (Compound 62-2)

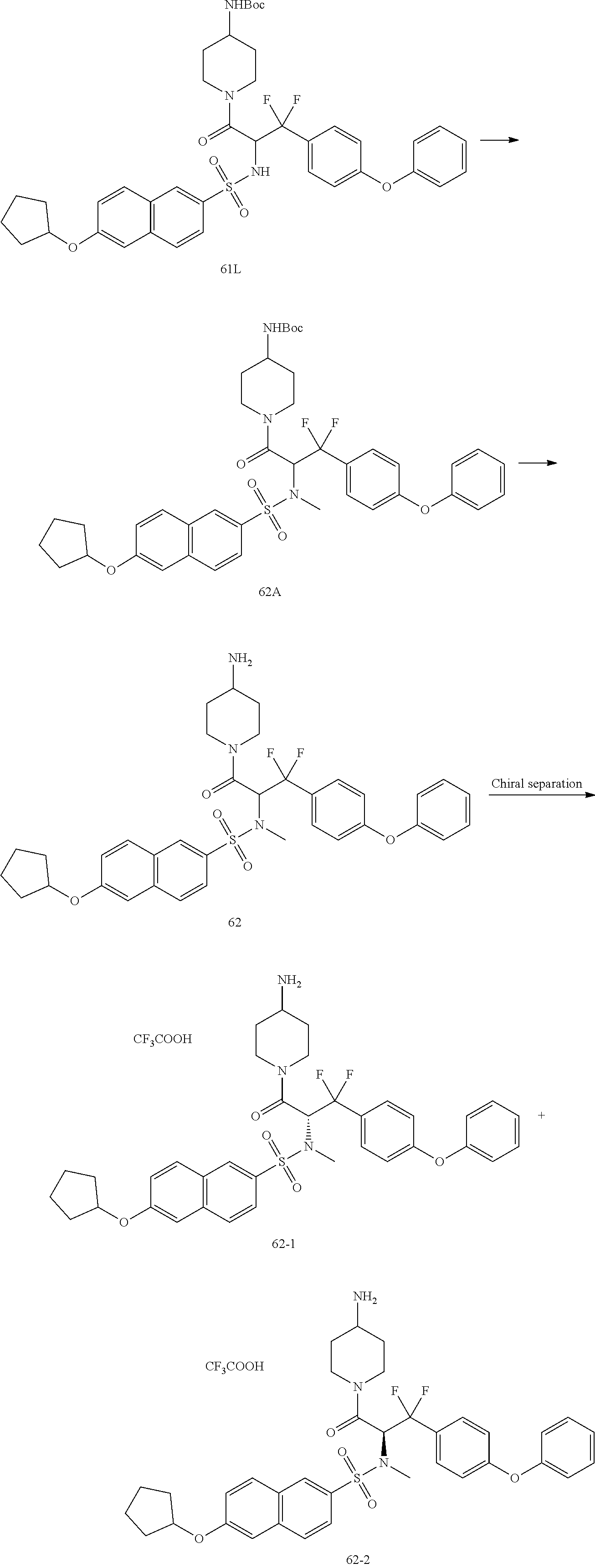

Compounds 62A and 62 were synthesized by employing the procedures described for Compounds 34A and 1 using Compounds 61L and 62A in lieu of Compounds 5A and 1A.

Compound 62A was directly used for the next step without further purification. LC-MS (ESI) m/z: 708 [M−55]⁺.

Compound 62, which was separated with chiral HPLC (Chiral separation condition: MeOH (0.5% NH₄OH); IC, 250×4.6 mm, 5 m) to furnish Compound 62-1 and Compound 62-1.

Compound 62-1: LC-MS (ESI) m/z: 664 [M+H]⁺; ¹H-NMR (CD₃OD, 400 MHz): δ (ppm) 1.30-1.92 (m, 8H), 2.00-2.22 (m, 4H), 2.55-2.74 (m, 1H), 3.07-3.44 (m, 5H), 4.33-4.63 (m, 2H), 4.92-5.02 (m, 1H), 5.75-5.81 (m, 1H), 6.85-7.03 (m, 4H), 7.18-7.27 (m, 2H), 7.31-7.42 (m, 4H), 7.50-7.60 (m, 2H), 7.84-8.24 (m, 3H). Chiral separation condition: n-hexane contained 0.5% NH₄OH; IC (250×4.6 mm, 5 μm); retention time: 6.16 minute.

Compound 62-2: LC-MS (ESI) m/z: 664 [M+H]⁺; ¹H-NMR (CD₃OD, 400 MHz): δ (ppm) 1.30-1.92 (m, 8H), 2.00-2.22 (m, 4H), 2.55-2.74 (m, 1H), 3.07-3.44 (m, 5H), 4.33-4.63 (m, 2H), 4.92-5.02 (m, 1H), 5.75-5.81 (m, 1H), 6.85-7.03 (m, 4H), 7.18-7.27 (m, 2H), 7.31-7.42 (m, 4H), 7.50-7.60 (m, 2H), 7.84-8.24 (m, 3H). Chiral separation condition: n-hexane contained 0.5% NH₄OH; IC (250×4.6 mm, 5 μm); retention time: 6.545 minute.

### Example 63

### Synthesis of (R)—N-(3-(4-aminopiperidin-1-yl)-1,1-difluoro-3-oxo-1-(4-phenoxy phenyl)propan-2-yl)-4-(cyclohexylmethoxy)benzenesulfonamide trifluoroacetate (Compound 63-1), and (S)—N-(3-(4-aminopiperidin-1-yl)-1,1-difluoro-3-oxo-1-(4-phenoxyphenyl) propan-2-yl)-4-(cyclohexylmethoxy)benzenesulfonamide trifluoroacetate (Compound 63-2)

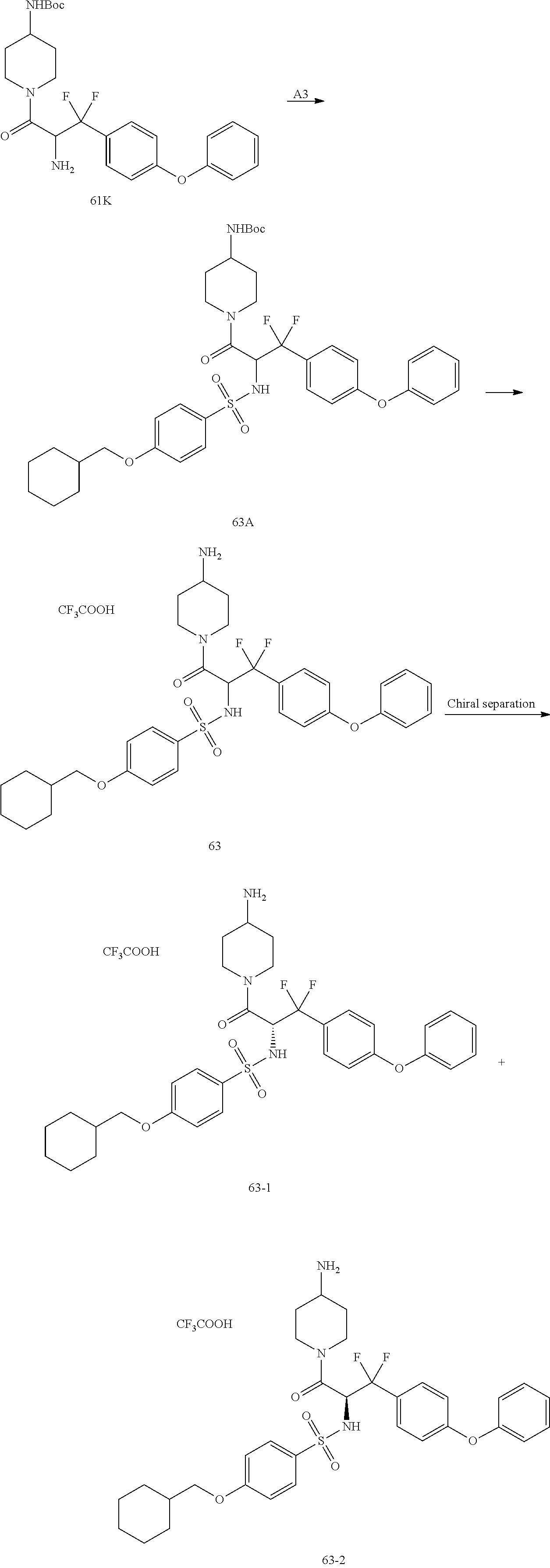

Compounds 63A and 63 were synthesized by employing the procedures described for Compounds 2D and 1 using Compound A3, Compounds 61K, and 63A in lieu of Compounds 2C, Compound B2, and 1A.

Compound 63A: LC-MS (ESI) m/z: 672 [M−55]⁺.

Compound 63 was separated with chiral HPLC (Chiral separation condition: MeOH contained 0.1% DEA; IC, 250×4.6 mm, 5 m) to furnish Compound 63-1 and Compound 63-2.

Compound 63-1: LC-MS (ESI) m/z: 628 [M+H]⁺; ¹H-NMR (CD₃OD, 400 MHz): δ (ppm) 0.91-1.89 (m, 13H), 1.98-2.12 (m, 2H), 2.41-.77 (m, 1H), 3.00-3.15 (m, 1H), 3.34-3.49 (m, 1H), 3.86 (d, J=6.4 Hz, 2H), 4.16-4.56 (m, 2H), 4.52-5.01 (m, 1H), 6.88-6.99 (m, 4H), 7.04-7.22 (m, 3H), 7.34-7.46 (m, 4H), 7.56-7.64 (m, 2H). Chiral separation condition: MeOH contained 0.5% NH₄OH; IC (250×4.6 mm, 5 μm); retention time: 6.97 minute.

Compound 63-2: LC-MS (ESI) m/z: 628 [M+H]⁺; ¹H-NMR (CD₃OD, 400 MHz): δ (ppm) 0.91-1.89 (m, 13H), 1.98-2.12 (m, 2H), 2.41-.77 (m, 1H), 3.00-3.15 (m, 1H), 3.34-3.49 (m, 1H), 3.86 (d, J=6.4 Hz, 2H), 4.16-4.56 (m, 2H), 4.52-5.01 (m, 1H), 6.88-6.99 (m, 4H), 7.04-7.22 (m, 3H), 7.34-7.46 (m, 4H), 7.56-7.64 (m, 2H). Chiral separation condition: MeOH contained 0.5% NH₄OH; IC (250×4.6 mm, 5 μm); retention time: 5.09 minute.

### Example 64

### Synthesis of (R)—N-(3-(4-aminopiperidin-1-yl)-1,1-difluoro-3-oxo-1-(4-phenoxy phenyl)propan-2-yl)-4-(cyclohexylmethoxy)-N-methylbenzenesulfonamide (Compound 64-1), and (S)—N-(3-(4-aminopiperidin-1-yl)-1,1-difluoro-3-oxo-1-(4-phenoxyphenyl)propan-2-yl)-4-(cyclohexylmethoxy)-N-methylbenzenesulfonamide (Compound 64-2)

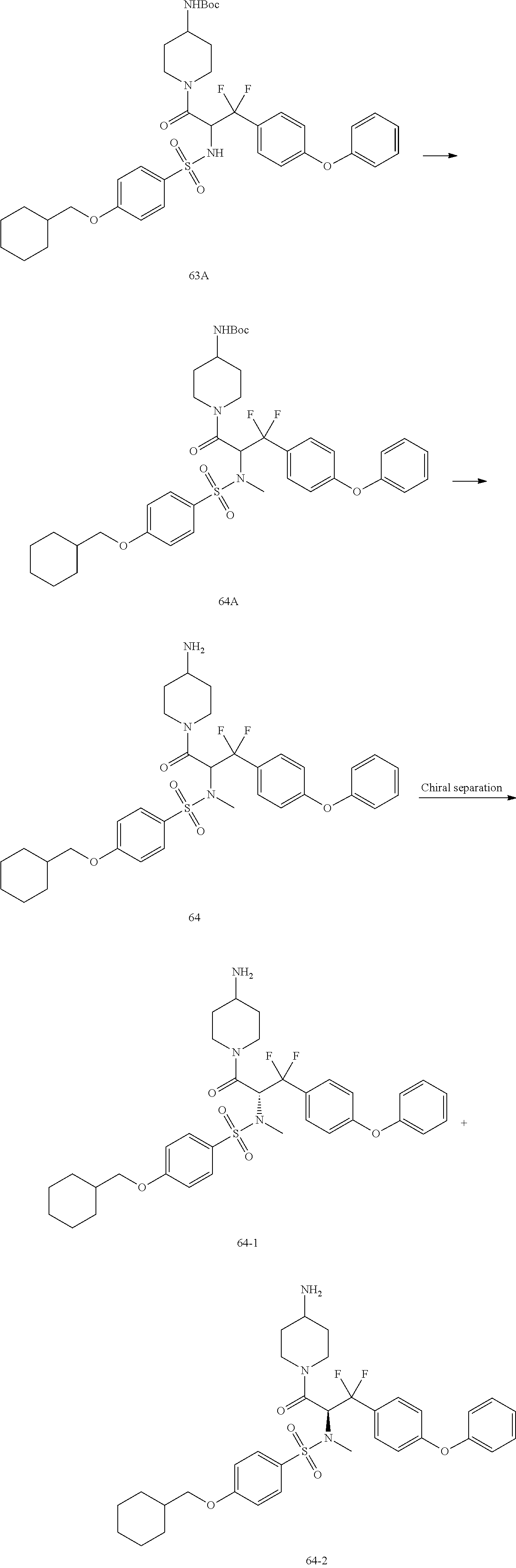 [SMILES: CC(C)(C)OC(=O)NC1CCN(C(=O)C(NS(=O)(=O)c2ccc(OCC3CCCCC3)cc2)C(F)(F)c2ccc(Oc3ccccc3)cc2)CC1 ; CN(C(C(=O)N1CCC(NC(=O)OC(C)(C)C)CC1)C(F)(F)c1ccc(Oc2ccccc2)cc1)S(=O)(=O)c1ccc(OCC2CCCCC2)cc1 ; CN(C(C(=O)N1CCC(N)CC1)C(F)(F)c1ccc(Oc2ccccc2)cc1)S(=O)(=O)c1ccc(OCC2CCCCC2)cc1 ; CN([C@H](C(=O)N1CCC(N)CC1)C(F)(F)c1ccc(Oc2ccccc2)cc1)S(=O)(=O)c1ccc(OCC2CCCCC2)cc1 ; CN([C@@H](C(=O)N1CCC(N)CC1)C(F)(F)c1ccc(Oc2ccccc2)cc1)S(=O)(=O)c1ccc(OCC2CCCCC2)cc1]

Compounds 64A and 64 were synthesized by employing the procedures described for Compounds 34A and 1 using Compounds 63A and 64A in lieu of Compounds 5A and 1A.

Compound 64A was directly used for the next step without further purification. LC-MS (ESI) m/z: 764 [M+Na]⁺.

Compound 64 was separated with chiral HPLC (Chiral separation condition: MeOH contained 0.5% NH₄OH; IC, 150×4.6 mm, 5 m) to furnish Compound 64-1 and Compound 64-2.

Compound 64-1: LC-MS (ESI) m/z: 642 [M+H]⁺; ¹H-NMR (CD₃OD, 400 MHz): δ (ppm) 0.78-1.51 (m, 8H), 1.70-1.88 (m, 8H), 2.61-2.69 (m, 1H), 2.87-3.25 (m, 5H), 3.85-3.97 (m, 2H), 4.02-4.39 (m, 2H), 5.63-5.73 (m, 1H), 6.97-7.08 (m, 6H), 7.18-7.22 (m, 1H), 7.39-7.516 (m, 4H), 7.60-7.67 (m, 2H). Chiral separation condition: MeOH contained 0.5% NH₄OH; IC (150×4.6 mm, 5 μm); retention time: 3.31 minute.

Compound 64-2: LC-MS (ESI) m/z: 642 [M+H]⁺; ¹H-NMR (CD₃OD, 400 MHz): δ (ppm) 0.78-1.51 (m, 8H), 1.70-1.88 (m, 8H), 2.61-2.69 (m, 1H), 2.87-3.25 (m, 5H), 3.85-3.97 (m, 2H), 4.02-4.39 (m, 2H), 5.63-5.73 (m, 1H), 6.97-7.08 (m, 6H), 7.18-7.22 (m, 1H), 7.39-7.516 (m, 4H), 7.60-7.67 (m, 2H). Chiral separation condition: MeOH contained 0.5% NH₄OH; IC (150×4.6 mm, 5 μm); retention time: 2.66 minute.

### Example 65

### Synthesis of N-((2R)-3-(3-amino-8-azabicyclo[3.2.1]octan-8-yl)-1-(3-bromophenyl)-1,1-difluoro-3-oxopropan-2-yl)-6-(cyclopentyloxy)naphthalene-2-sulfonamide (Compound 65-1), and N-((2S)-3-(3-amino-8-azabicyclo[3.2.1]octan-8-yl)-1-(3-bromophenyl)-1,1-difluoro-3-oxopropan-2-yl)-6-(cyclopentyloxy)naphthalene-2-sulfonamide (Compound 65-2)

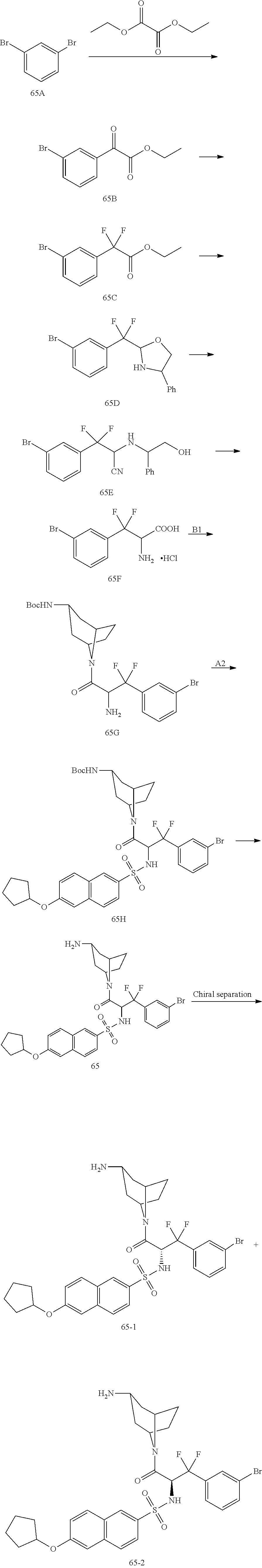

Compounds 65B, 65C, 65D, 65E, 65F, 65G, 65H, and 65 were synthesized by employing the procedures described for Compound 9B, Compounds B2-3, B2-4, B2-5, B2-6, B2, Compounds 2D, and 1 using Compounds 65A, 65B, 65C, 65D, 65E, 65F, 65G, Compound A2, and Compound 65H in lieu of Compound 9A, Compounds B2-2, B2-3, B2-4, B2-5, B2-6, Compounds 2C, and 1A.

Compound 65B: LC-MS (ESI) m/z: 257 [M+H]⁺; ¹H-NMR (CDCl₃, 400 MHz): δ (ppm) 1.42 (t, J=7.2 Hz, 3H), 4.47 (q, J=7.2 Hz, 2H), 7.41 (t, J=8.0 Hz, 1H), 7.79 (d, J=8.0 Hz, 1H), 7.97 (d, J=8.0 Hz, 1H), 8.18 (s, 1H).

Compound 65C: LC-MS (ESI) m/z: 279 [M+H]⁺; ¹H-NMR (CDCl₃, 400 MHz): δ (ppm) 1.33 (t, J=7.2 Hz, 3H), 4.32 (q, J=7.2 Hz, 2H), 7.35 (t, J=8.0 Hz, 1H), 7.56 (d, J=8.0 Hz, 1H), 7.64 (d, J=8.0 Hz, 1H), 7.77 (s, 1H).

Compound 65D: LC-MS (ESI) m/z: 354 [M+H]⁺; ¹H-NMR (CDCl₃, 400 MHz): δ (ppm) 2.62-2.66 (m, 1H), 3.26-3.30 (m, 1H), 4.12-4.50 (m, 2H), 5.12-5.17 (m, 1H), 7.24-7.35 (m, 6H), 7.56-7.62 (m, 2H), 7.79-7.82 (m, 1H).

Compound 65E: LC-MS (ESI) m/z: 381 [M+H]⁺; ¹H-NMR (CDCl₃, 400 MHz): δ (ppm) 1.78-1.81 (m, 1H), 2.64-2.67 (m, 1H), 3.47-3.54 (m, 1H), 3.74-3.80 (m, 2H), 4.05-4.08 (m, 1H), 7.00-7.01 (m, 1H), 7.24-7.44 (m, 6H), 7.60 (s, 1H), 7.67-7.69 (m, 1H).

Compound 65F: LC-MS (ESI) m/z: 280 [M+H]⁺; ¹H-NMR (DMSO-d₆, 400 MHz): δ (ppm) 4.64-4.71 (m, 1H), 7.47 (t, J=7.6 Hz, 1H), 7.59 (d, J=7.6 Hz, 1H), 7.75-7.78 (m, 2H).

Compound 65G: LC-MS (ESI) m/z: 488 [M+H]⁺.

Compound 65H: LC-MS (ESI) m/z: 706 [M−55]⁺.

Compound 65 was separated with chiral HPLC to furnish Compound 65-1 and Compound 65-2.

Compound 65-1: LC-MS (ESI) m/z: 662 [M+H]⁺; ¹H-NMR (CD₃OD, 400 MHz): δ (ppm) 1.21-2.09 (m, 16H), 3.43-3.61 (m, 1H), 4.24-4.62 (m, 3H), 4.89-4.92 (m, 1H), 6.88-7.07 (m, 1H), 7.10-7.56 (m, 6H), 7.65-7.75 (m, 2H), 8.00-8.06 (m, 1H). Chiral separation condition: MeOH contained 0.5% NH₄OH; IC (4.6×250 mm, 5 μm); retention time: 5.53 minute.

Compound 65-2: LC-MS (ESI) m/z: 662 [M+H]⁺; ¹H-NMR (CD₃OD, 400 MHz): δ (ppm) 1.38-1.94 (m, 16H), 2.70-2.90 (m, 1H), 4.04-4.28 (m, 2H), 4.55-4.63 (m, 1H), 4.87-4.91 (m, 1H), 7.10-7.23 (m, 3H), 7.35-7.80 (m, 6H), 8.09-8.15 (m, 1H). Chiral separation condition: MeOH contained 0.5% NH₄OH; IC (4.6×250 mm, 5 μm); retention time: 4.34 minute.

### Example 66

### Synthesis of N-((2R)-1-([1,1′-biphenyl]-3-yl)-3-(3-amino-8-azabicyclo[3.2.1]octan-8-yl)-1,1-difluoro-3-oxopropan-2-yl)-6-(cyclopentyloxy)naphthalene-2-sulfonamide (Compound 66-1), and N-((2S)-1-([1,1′-biphenyl]-3-yl)-3-(3-amino-8-azabicyclo[3.2.1]octan-8-yl)-1,1-difluoro-3-oxopropan-2-yl)-6-(cyclopentyloxy)naphthalene-2-sulfonamide (Compound 66-2)

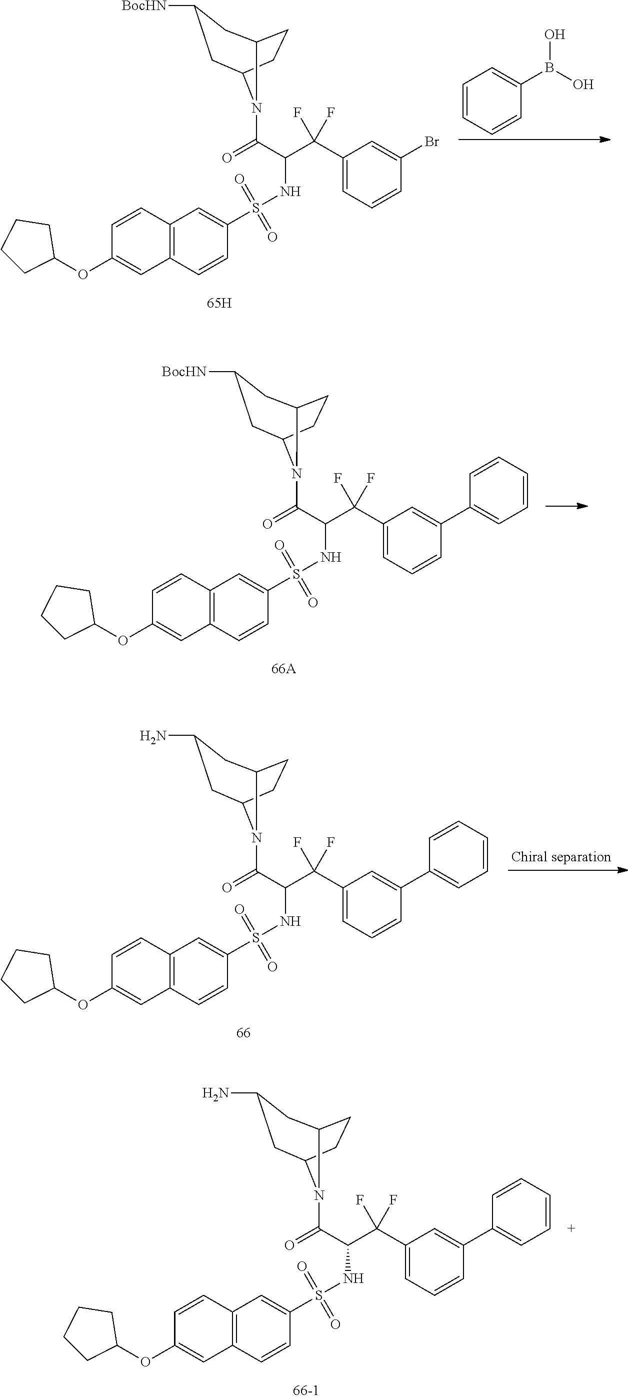

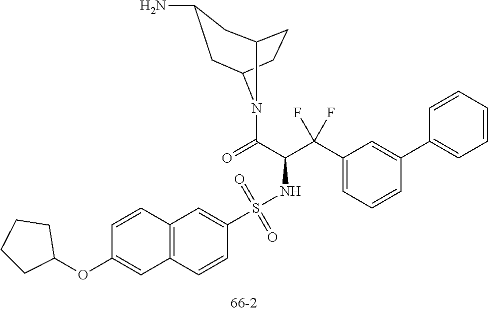 [SMILES: NC1CC2CCC(C1)N2C(=O)[C@H](NS(=O)(=O)c1ccc2cc(OC3CCCC3)ccc2c1)C(F)(F)c1cccc(-c2ccccc2)c1]

Compounds 66A and 66 were synthesized by employing the procedures described for Compounds 14C and 1 using phenylboronic acid, Compounds 65H, and 66A in lieu of Compounds 14B, 14A, and 1A.

Compound 66A: LC-MS (ESI) m/z: 704 [M−55]⁺.

Compound 66 was separated with chiral HPLC to furnish Compound 66-1 and Compound 66-2.

Compound 66-1: LC-MS (ESI) m/z: 660 [M+H]⁺; ¹H-NMR (CD₃OD, 400 MHz): δ (ppm) 1.20-1.78 (m, 13H), 1.91-1.94 (m, 3H), 2.66-2.86 (m, 1H), 4.00-4.25 (m, 2H), 4.67-4.71 (m, 1H), 4.84-4.87 (m, 1H), 7.07-7.15 (m, 2H), 7.26-7.78 (m, 12H), 8.10-8.17 (m, 1H). Chiral separation condition: MeOH contained 0.5% NH₄OH; IC (4.6×250 mm, 5 μm); retention time: 8.51 minute.

Compound 66-2: LC-MS (ESI) m/z: 660 [M+H]⁺; ¹H-NMR (CD₃OD, 400 MHz): δ (ppm) 1.18-1.60 (m, 8H), 1.73-1.76 (m, 5H), 1.89-1.94 (m, 3H), 3.25-3.31 (m, 1H), 4.26-4.46 (m, 2H), 4.64-4.70 (m, 1H), 4.82-4.84 (m, 1H), 7.05-7.08 (m, 2H), 7.23-7.62 (m, 11H), 7.62-7.70 (m, 1H), 8.06 (s, 1H). Chiral separation condition: MeOH contained 0.5% NH₄OH; IC (4.6×250 mm, 5 μm); retention time: 6.22 minute.

### Example 67

### Synthesis of N-((2R)-3-(3-amino-8-azabicyclo[3.2.1]octan-8-yl)-1-(3-bromophenyl)-1,1-difluoro-3-oxopropan-2-yl)-6-(cyclopentyloxy)-N-methylnaphthalene-2-sulfonamide (Compound 67-1), and N-((2S)-3-(3-amino-8-azabicyclo[3.2.1]octan-8-yl)-1-(3-bromophenyl)-1,1-difluoro-3-oxopropan-2-yl)-6-(cyclopentyloxy)-N-methylnaphthalene-2-sulfonamide (Compound 67-2)

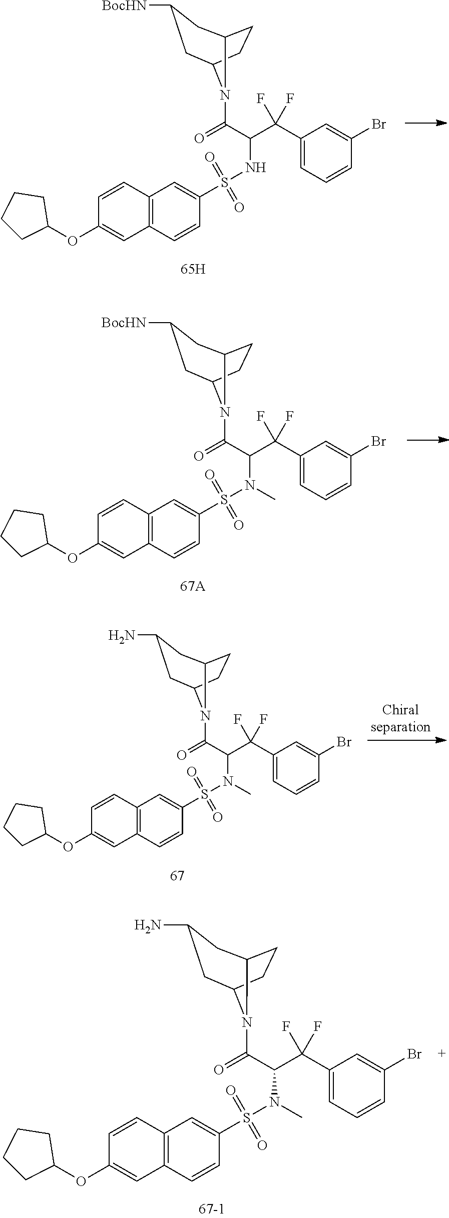 [SMILES: CC(C)(C)OC(=O)NC1CC2CCC(C1)N2C(=O)C(NS(=O)(=O)c1ccc2cc(OC3CCCC3)ccc2c1)C(F)(F)c1cccc(Br)c1 ; CN(C(C(=O)N1C2CCC1CC(NC(=O)OC(C)(C)C)C2)C(F)(F)c1cccc(Br)c1)S(=O)(=O)c1ccc2cc(OC3CCCC3)ccc2c1 ; CN(C(C(=O)N1C2CCC1CC(N)C2)C(F)(F)c1cccc(Br)c1)S(=O)(=O)c1ccc2cc(OC3CCCC3)ccc2c1 ; CN([C@H](C(=O)N1C2CCC1CC(N)C2)C(F)(F)c1cccc(Br)c1)S(=O)(=O)c1ccc2cc(OC3CCCC3)ccc2c1]

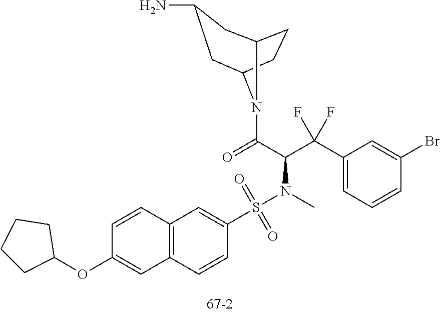 [SMILES: CN([C@@H](C(=O)N1C2CCC1CC(N)C2)C(F)(F)c1cccc(Br)c1)S(=O)(=O)c1ccc2cc(OC3CCCC3)ccc2c1]

Compounds 67A and 67 were synthesized by employing the procedures described for Compounds 34A and 1 using Compounds 65H and 67A in lieu of Compounds 5A and 1A.

Compound 67A: LC-MS (ESI) m/z: 798 [M+Na]⁺.

Compound 67 was separated with chiral HPLC to furnish Compound 67-1 and Compound 67-2.

Compound 67-1: LC-MS (ESI) m/z: 676 [M+H]⁺; ¹H-NMR (CD₃OD, 400 MHz): δ (ppm) 1.48-2.05 (m, 16H), 2.89-3.10 (m, 3H), 3.53-3.61 (m, 1H), 4.44-4.60 (m, 2H), 4.90-4.92 (m, 1H), 5.42-5.52 (m, 1H), 7.12-7.21 (m, 3H), 7.28-7.33 (m, 1H), 7.40-7.44 (m, 2H), 7.49-7.54 (m, 1H), 7.71-7.84 (m, 2H), 8.01-8.16 (m, 1H). Chiral separation condition: MeOH contained 0.5% NH₄OH; AD-H (4.6×250 mm, 5 μm); retention time: 6.27 minute.

Compound 67-2: LC-MS (ESI) m/z: 676 [M+H]⁺; ¹H-NMR (CD₃OD, 400 MHz): δ (ppm) 1.29-2.06 (m, 16H), 3.11-3.18 (m, 1H), 3.07-3.24 (m, 3H), 4.30-4.57 (m, 2H), 4.99-5.02 (m, 1H), 5.44-5.59 (m, 1H), 7.25-7.39 (m, 3H), 7.44-7.66 (m, 4H), 7.89-7.94 (m, 2H), 8.24-8.28 (m, 1H). Chiral separation condition: MeOH contained 0.5% NH₄OH; AD-H (4.6×250 mm, 5 μm); retention time: 3.17 minute.

### Example 68

### Synthesis of N-((2R)-3-(3-amino-8-azabicyclo[3.2.1]octan-8-yl)-1-(4-bromophenyl)-1,1-difluoro-3-oxopropan-2-yl)-6-(cyclopentyloxy)-N-ethylnaphthalene-2-sulfonamide (Compound 68-1), and N-((2S)-3-(3-amino-8-azabicyclo[3.2.1]octan-8-yl)-1-(4-bromophenyl)-1,1-difluoro-3-oxopropan-2-yl)-6-(cyclopentyloxy)-N-ethylnaphthalene-2-sulfonamide (Compound 68-2)

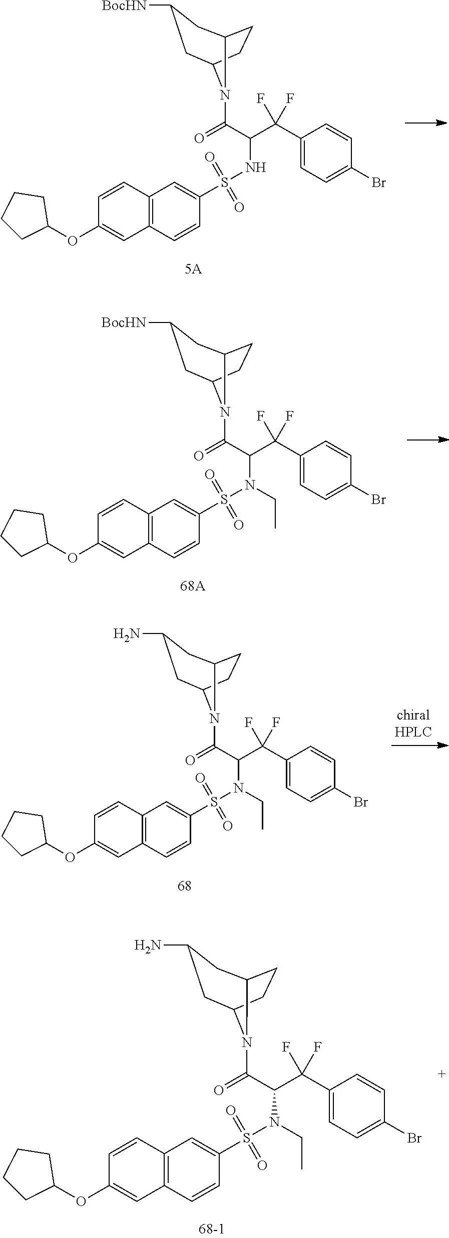 [SMILES: CC(C)(C)OC(=O)NC1CC2CCC(C1)N2C(=O)C(NS(=O)(=O)c1ccc2cc(OC3CCCC3)ccc2c1)C(F)(F)c1ccc(Br)cc1 ; CCN(C(C(=O)N1C2CCC1CC(NC(=O)OC(C)(C)C)C2)C(F)(F)c1ccc(Br)cc1)S(=O)(=O)c1ccc2cc(OC3CCCC3)ccc2c1 ; CCN(C(C(=O)N1C2CCC1CC(N)C2)C(F)(F)c1ccc(Br)cc1)S(=O)(=O)c1ccc2cc(OC3CCCC3)ccc2c1 ; CCN([C@H](C(=O)N1C2CCC1CC(N)C2)C(F)(F)c1ccc(Br)cc1)S(=O)(=O)c1ccc2cc(OC3CCCC3)ccc2c1]

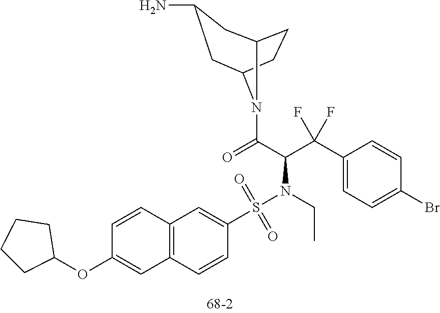 [SMILES: CCN([C@@H](C(=O)N1C2CCC1CC(N)C2)C(F)(F)c1ccc(Br)cc1)S(=O)(=O)c1ccc2cc(OC3CCCC3)ccc2c1]

Compounds 68A and 68 were synthesized by employing the procedures described for Compounds 34A and 1 using iodoethane and Compound 68A in lieu of iodomethane and Compound 1A.

Compound 68A: LC-MS (ESI) m/z: 813 [M+Na]⁺.

Compound 68 was separated with chiral HPLC to furnish Compound 68-1 and Compound 68-2.

Compound 68-1: LC-MS (ESI) m/z: 691 [M+H]⁺; ¹H-NMR (CD₃OD, 400 MHz): δ (ppm) 0.97-1.21 (m, 3H), 1.28-1.73 (m, 11H), 1.93-2.08 (m, 5H), 3.40-3.88 (m, 3H), 4.37-4.60 (m, 2H), 4.90 (s, 1H), 5.34-5.38 (m, 1H), 7.18-7.62 (m, 7H), 7.70-7.83 (m, 2H), 8.13-8.25 (m, 1H). Chiral separation condition: MeOH contained 0.5% NH₄OH; AD-H (4.6×250 mm, 5 μm); retention time: 6.07 minute.

Compound 68-2: LC-MS (ESI) m/z: 691 [M+H]⁺; ¹H-NMR (CD₃OD, 400 MHz): δ (ppm) 0.35-0.95 (m, 1H), 1.00-1.15 (m, 4H), 1.25-1.73 (m, 12H), 1.82-1.97 (m, 2H), 2.90-2.96 (m, 1H), 3.48-3.98 (m, 2H), 4.08-4.33 (m, 2H), 4.90 (s, 1H), 5.26-5.37 (m, 1H), 7.13-7.32 (m, 4H), 7.42-7.63 (m, 3H), 7.79-7.85 (m, 2H), 8.21-8.22 (m, 1H). Chiral separation condition: MeOH contained 0.5% NH₄OH; AD-H (4.6×250 mm, 5 μm); retention time: 3.95 minute.

### Example 69

### Synthesis of (R)—N-(1-([1,1′-biphenyl]-3-yl)-3-(4-aminopiperidin-1-yl)-1,1-difluoro-3-oxopropan-2-yl)-6-(cyclopentyloxy)naphthalene-2-sulfonamide trifluoroacetate (Compound 69-1), and (S)—N-(1-([1,1′-biphenyl]-3-yl)-3-(4-aminopiperidin-1-yl)-1,1-difluoro-3-oxopropan-2-yl)-6-(cyclopentyloxy)naphthalene-2-sulfonamide trifluoroacetate (Compound 69-2)

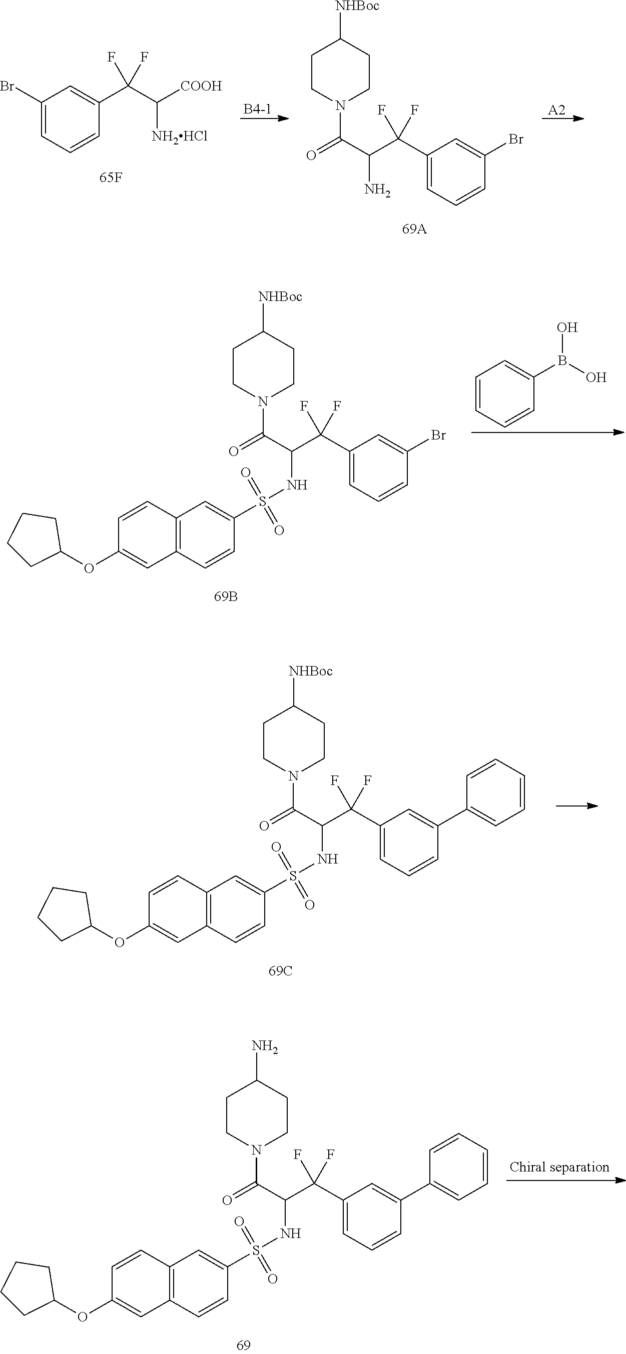

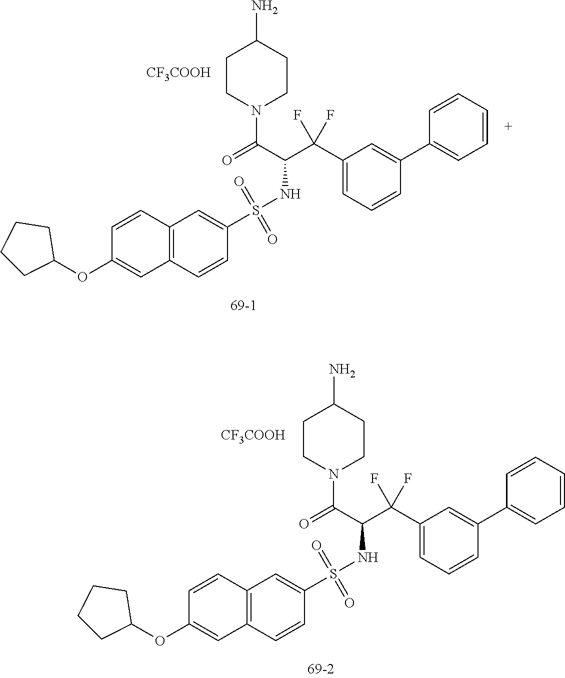 [SMILES: NC1CCN(C(=O)[C@@H](NS(=O)(=O)c2ccc3cc(OC4CCCC4)ccc3c2)C(F)(F)c2cccc(-c3ccccc3)c2)CC1 ; "CF3COOH" ; NC1CCN(C(=O)[C@H](NS(=O)(=O)c2ccc3cc(OC4CCCC4)ccc3c2)C(F)(F)c2cccc(-c3ccccc3)c2)CC1 ; "CF3COOH"]

Compounds 69A, 69B, 69C, and 69 were synthesized by employing the procedures described for Compound B4, Compounds 2D, 14C, and 1 using Compounds 65F, 69A, Compound A2, Compounds 69B, phenylboronic acid, and 69C in lieu of Compounds B2-6, B2, Compounds 2C, 14A, 14B, and 1A.

Compound 69A: LC-MS (ESI) m/z: 462 [M+H]⁺; ¹H-NMR (CDCl₃, 400 MHz): δ (ppm) 1.45-1.46 (m, 9H), 1.85-2.01 (m, 4H), 2.71-3.15 (m, 2H), 3.63-3.85 (m, 2H), 4.27-4.57 (m, 3H), 7.29-7.43 (m, 2H), 7.59-7.61 (m, 2H).

Compound 69B: LC-MS (ESI) m/z: 680 [M−55]⁺.

Compound 69C: LC-MS (ESI) m/z: 678 [M−55]⁺.

Compound 69 was separated with chiral HPLC to furnish Compound 69-1 and Compound 69-2.

Compound 69-1: LC-MS (ESI) m/z: 634 [M+H]⁺; ¹H-NMR (CD₃OD, 400 MHz): δ (ppm) 1.26-2.03 (m, 12H), 2.76-3.40 (m, 3H), 4.17-4.58 (m, 2H), 4.94-4.96 (m, 1H), 5.03-5.16 (m, 1H), 7.13-7.21 (m, 2H), 7.36-7.84 (m, 12H), 8.05-8.16 (m, 1H). Chiral separation condition: MeOH contained 0.1% NH₄OH; OJ-H (4.6×250 mm, 5 μm); retention time: 2.37 minute.

Compound 69-2: LC-MS (ESI) m/z: 634 [M+H]⁺; ¹H-NMR (CD₃OD, 400 MHz): δ (ppm) 1.25-2.03 (m, 12H), 2.72-3.42 (m, 3H), 4.16-4.58 (m, 2H), 4.89-4.97 (m, 1H), 5.03-5.16 (m, 1H), 7.13-7.20 (m, 2H), 7.36-7.84 (m, 12H), 8.05-8.16 (m, 1H). Chiral separation condition: MeOH contained 0.1% NH₄OH; OJ-H (4.6×250 mm, 5 μm); retention time: 3.30 minute.

### Example 70

### Synthesis of N-((2R)-1-([1,1′-biphenyl]-3-yl)-3-(3-amino-8-azabicyclo[3.2.1]octan-8-yl)-1,1-difluoro-3-oxopropan-2-yl)-4-(cyclohexylmethoxy)benzenesulfonamide trifluoroacetate (Compound 70-1), and N-((2S)-1-([1,1′-biphenyl]-3-yl)-3-(3-amino-8-azabicyclo[3.2.1]octan-8-yl)-1,1-difluoro-3-oxopropan-2-yl)-4-(cyclohexylmethoxy) benzenesulfonamide trifluoroacetate (Compound 70-2)

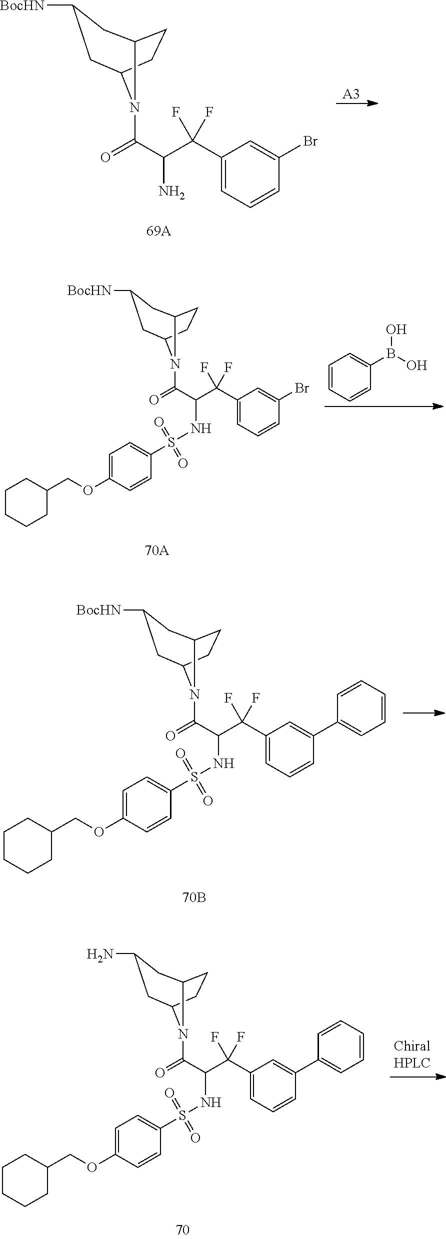

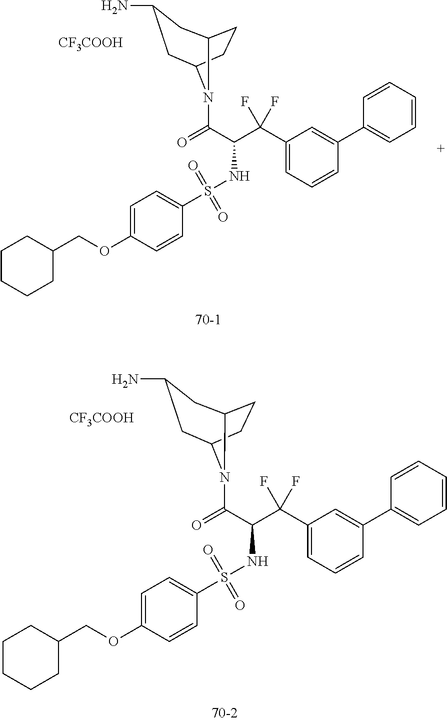 [SMILES: NC1CC2CCC(C1)N2C(=O)[C@@H](NS(=O)(=O)c1ccc(OCC2CCCCC2)cc1)C(F)(F)c1cccc(-c2ccccc2)c1 ; "CF3COOH" ; NC1CC2CCC(C1)N2C(=O)[C@H](NS(=O)(=O)c1ccc(OCC2CCCCC2)cc1)C(F)(F)c1cccc(-c2ccccc2)c1 ; "CF3COOH"]

Compounds 70A, 70B, and 70 were synthesized by employing the procedures described for Compounds 2D, 14C, and 1 using Compound A3, Compounds 69A, 70A, phenylboronic acid, and 70B in lieu of Compound B2, Compounds 2C, 14A, 14B, and 1A.

Compound 70A: LC-MS (ESI) m/z: 684 [M−55]⁺.

Compound 70B: LC-MS (ESI) m/z: 682 [M−55]⁺.

Compound 70 was separated with chiral HPLC to furnish Compound 70-1 as and Compound 70-2.

Compound 70-1: LC-MS (ESI) m/z: 638 [M+H]⁺; ¹H-NMR (CD₃OD, 400 MHz): δ (ppm) 1.02-1.11 (m, 2H), 1.26-1.35 (m, 3H), 1.74-2.15 (m, 14H), 3.66-3.72 (m, 3H), 4.55-4.66 (m, 2H), 4.71-4.87 (m, 1H), 6.79-6.86 (m, 2H), 7.39-7.70 (m, 11H). Chiral separation condition: MeOH (0.1% NH₄OH); AS-H (4.6×250 mm, 5 μm); retention time: 3.57 minute.

Compound 70-2: LC-MS (ESI) m/z: 638 [M+H]⁺; ¹H-NMR (CD₃OD, 400 MHz): δ (ppm) 1.02-1.11 (m, 2H), 1.23-1.38 (m, 3H), 1.72-2.14 (m, 14H), 3.66-3.72 (m, 3H), 4.55-4.66 (m, 2H), 4.71-4.87 (m, 1H), 6.79-6.86 (m, 2H), 7.39-7.70 (m, 11H). Chiral separation condition: MeOH (0.1% NH₄OH); AS-H (4.6×250 mm, 5 μm); retention time: 2.97 minute.

### Example 71

### Synthesis of (R)—N-(3-(4-aminopiperidin-1-yl)-1,1-difluoro-3-oxo-1-(4-(trifluoromethyl)phenyl)propan-2-yl)-6-(cyclopentyloxy)naphthalene-2-sulfonamide (Compound 71-1), and (S)—N-(3-(4-aminopiperidin-1-yl)-1,1-difluoro-3-oxo-1-(4-(trifluoromethyl)phenyl)propan-2-yl)-6-(cyclopentyloxy)naphthalene-2-sulfonamide (Compound 71-2)

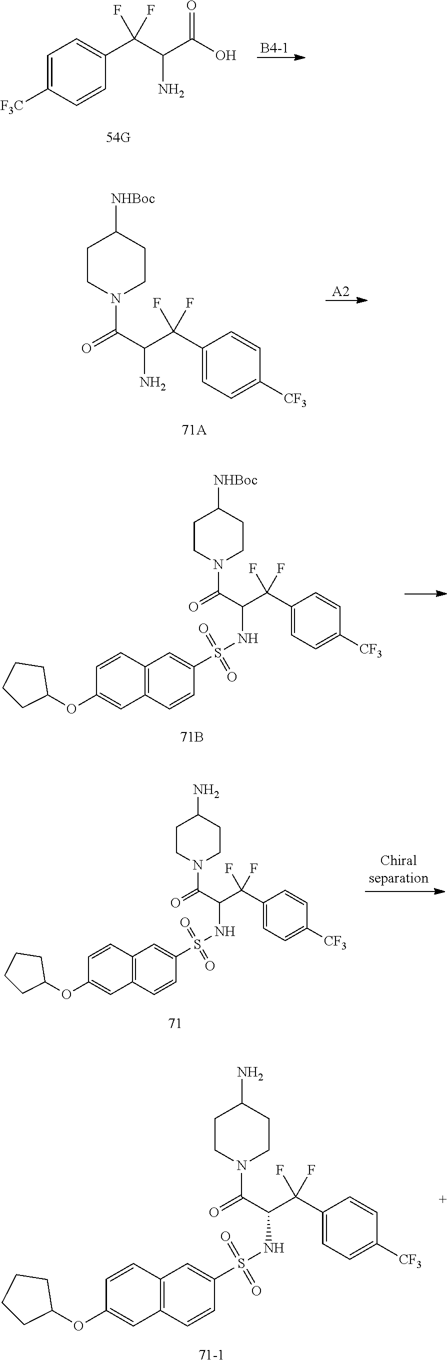

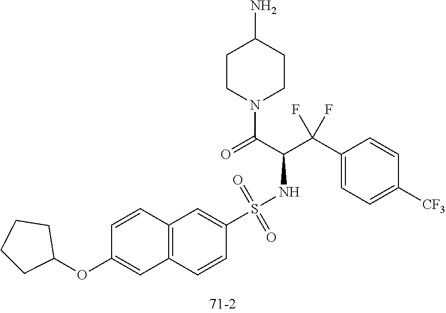 [SMILES: NC1CCN(C(=O)[C@H](NS(=O)(=O)c2ccc3cc(OC4CCCC4)ccc3c2)C(F)(F)c2ccc(C(F)(F)F)cc2)CC1]

Compounds 71A, 71B, and 71 were synthesized by employing the procedures described for Compound B4, Compounds 1A and 6 using Compound 54G, Compound A2, Compounds 71A, and 71B in lieu of Compounds B2-6, A1, B2, and Compound 6C.

Compound 71A: LC-MS (ESI) m/z: 452 [M+H]⁺; ¹H-NMR (CDCl₃, 400 MHz): δ (ppm) 1.13-1.38 (m, 2H), 1.44 (s, 9H), 1.93-2.00 (m, 2H), 2.71-2.81 (m, 1H), 2.88-3.17 (m, 1H), 3.60-3.70 (m, 1H), 3.81-3.89 (m, 1H), 4.35-4.55 (m, 3H), 7.57-7.61 (m, 2H), 7.68-7.72 (m, 2H).

Compound 71B: LC-MS (ESI) m/z: 626 [M−Boc+H]⁺.

Compound 71 was separated with chiral HPLC to give Compound 71-1 and Compound 71-2.

Compound 71-1: LC-MS (ESI) m/z: 626 [M+H]⁺; ¹H-NMR (CD₃OD, 400 MHz): δ (ppm) 0.03-0.34 (m, 1H), 0.73-0.92 (m, 1H), 1.19-1.25 (m, 1H), 1.35-1.50 (m, 2H), 1.56-1.62 (m, 2H), 1.66-1.82 (m, 4H), 1.89-1.97 (m, 2H), 2.27-2.47 (m, 1H), 2.61-2.73 (m, 1H), 3.71-4.00 (m, 2H), 4.77-4.89 (m, 2H), 7.08-7.19 (m, 2H), 7.45-7.59 (m, 5H), 7.65-7.72 (m, 1H), 7.74-7.79 (m, 1H), 8.09-8.13 (m, 1H). Chiral separation condition: MeOH contained 0.5% NH₄OH; (R, R)-Whelk-O1 (4.6×250 mm, 5 μm); retention time: 4.17 minute.

Compound 71-2: LC-MS (ESI) m/z: 626 [M+H]⁺; ¹H-NMR (CD₃OD, 400 MHz): δ (ppm) 0.03-0.37 (m, 1H), 0.76-0.95 (m, 1H), 1.23-1.27 (m, 1H), 1.37-1.52 (m, 2H), 1.58-1.64 (m, 2H), 1.71-1.85 (m, 4H), 1.92-2.00 (m, 2H), 2.30-2.49 (m, 1H), 2.64-2.76 (m, 1H), 3.74-4.03 (m, 2H), 4.80-4.91 (m, 2H), 7.11-7.22 (m, 2H), 7.48-7.62 (m, 5H), 7.67-7.74 (m, 1H), 7.77-7.82 (m, 1H), 8.11-8.16 (m, 1H). Chiral separation condition: MeOH contained 0.5% NH₄OH; (R, R)-Whelk-O1 (4.6×250 mm, 5 μm); retention time: 4.99 minute.

### Example 72

### Synthesis of (R)—N-(3-(4-aminopiperidin-1-yl)-1,1-difluoro-3-oxo-1-(4-(trifluoromethyl)phenyl)propan-2-yl)-4-(cyclohexylmethoxy)benzenesulfonamide (Compound 72-1), and (S)—N-(3-(4-aminopiperidin-1-yl)-1,1-difluoro-3-oxo-1-(4-(trifluoromethyl)phenyl)propan-2-yl)-4-(cyclohexylmethoxy)benzenesulfonamide (Compound 72-2)

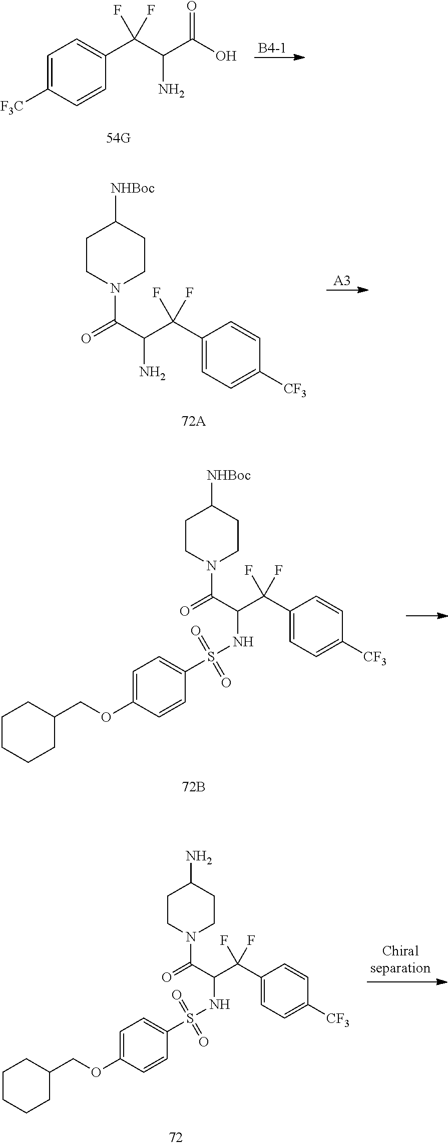

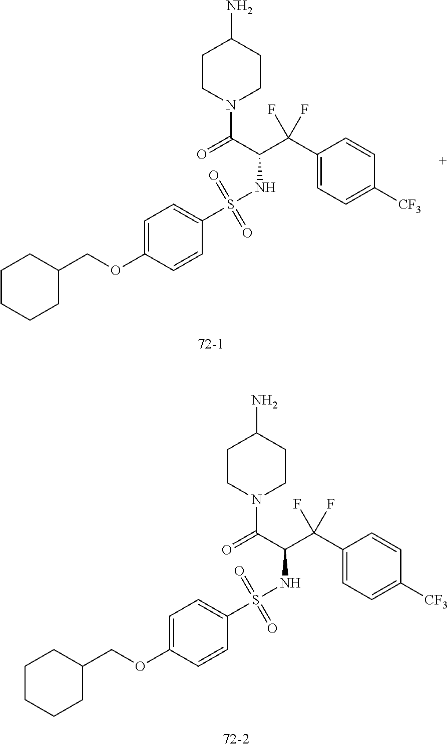 [SMILES: NC1CCN(C(=O)[C@@H](NS(=O)(=O)c2ccc(OCC3CCCCC3)cc2)C(F)(F)c2ccc(C(F)(F)F)cc2)CC1 ; NC1CCN(C(=O)[C@H](NS(=O)(=O)c2ccc(OCC3CCCCC3)cc2)C(F)(F)c2ccc(C(F)(F)F)cc2)CC1]

Compounds 72A, 72B and 72 were synthesized by employing the procedures described for Compounds B4, 1A and 6 using Compounds 54G, A3, 72A, and 72B in lieu of Compounds B2-6, A1, B2, and 6C.

Compound 72B: LC-MS (ESI) m/z: 604 [M−Boc+H]⁺.

Compound 72, which was separated with chiral HPLC to give Compound 72-1 (yield 5.6%) as a white solid and Compound 72-2 (yield 5.0%) as a white solid.

Compound 72-1: LC-MS (ESI) m/z: 604 [M+H]⁺; ¹H-NMR (CD₃OD, 400 MHz): δ (ppm) 0.78-1.28 (m, 7H), 1.60-1.79 (m, 8H), 2.27-2.58 (m, 1H), 2.73-2.94 (m, 2H), 3.74 (d, J=6.0 Hz, 2H), 3.87-3.95 (m, 1H), 4.02-4.20 (m, 1H), 4.81-4.86 (m, 1H), 6.84-6.88 (m, 2H), 7.49-7.66 (m, 6H). Chiral separation condition: MeOH contained 0.5% NH₄OH; OZ-H (4.6×250 mm, 5 μm); retention time: 6.64 minute.

Compound 72-2: LC-MS (ESI) m/z: 604 [M+H]⁺; ¹H-NMR (CD₃OD, 400 MHz): δ (ppm) 0.75-1.28 (m, 7H), 1.61-1.88 (m, 8H), 2.26-2.56 (m, 1H), 2.65-2.91 (m, 2H), 3.74 (d, J=6.0 Hz, 2H), 3.83-3.89 (m, 1H), 3.99-4.16 (m, 1H), 4.80-4.84 (m, 1H), 6.84-6.89 (m, 2H), 7.50-7.66 (m, 6H). Chiral separation condition: MeOH contained 0.5% NH₄OH; OZ-H (4.6×250 mm, 5 μm); retention time: 4.62 minute.

### Example 73

### Synthesis of N-((2R)-3-(3-amino-8-azabicyclo[3.2.1]octan-8-yl)-1,1-difluoro-3-oxo-1-(4-(trifluoromethyl)phenyl)propan-2-yl)-6-(cycyclopentyloxy)-N-methylnaphthalene-2-sulfonamide (Compound 73-1), and N-((2S)-3-(3-amino-8-azabicyclo[3.2.1]octan-8-yl)-1,1-difluoro-3-oxo-1-(4-(trifluoromethyl)phenyl)propan-2-yl)-6-(cyclopentyloxy)-N-methylnaphthalene-2-sulfonamide (Compound 73-2)

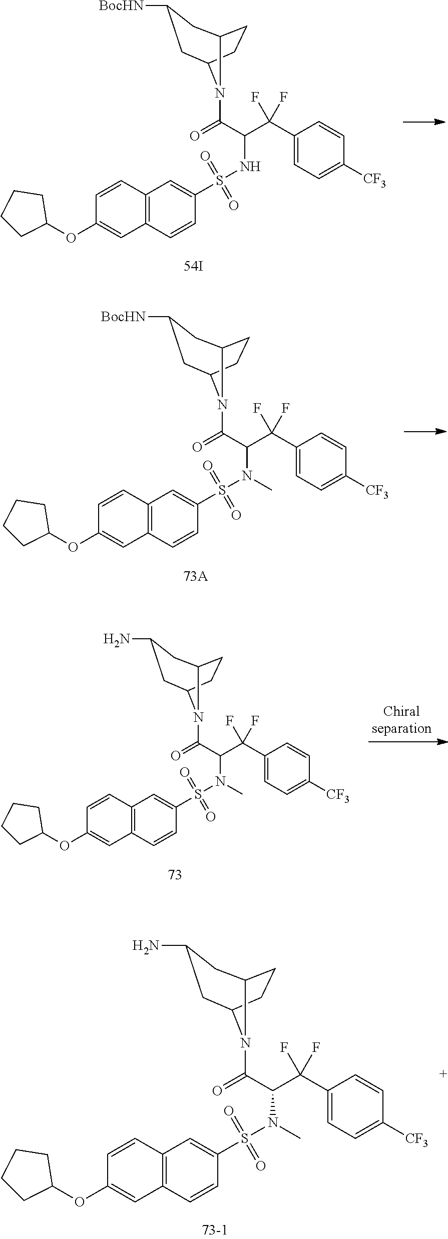 [SMILES: CC(C)(C)OC(=O)NC1CC2CCC(C1)N2C(=O)C(NS(=O)(=O)c1ccc2cc(OC3CCCC3)ccc2c1)C(F)(F)c1ccc(C(F)(F)F)cc1 ; CN(C(C(=O)N1C2CCC1CC(NC(=O)OC(C)(C)C)C2)C(F)(F)c1ccc(C(F)(F)F)cc1)S(=O)(=O)c1ccc2cc(OC3CCCC3)ccc2c1 ; CN(C(C(=O)N1C2CCC1CC(N)C2)C(F)(F)c1ccc(C(F)(F)F)cc1)S(=O)(=O)c1ccc2cc(OC3CCCC3)ccc2c1 ; CN([C@H](C(=O)N1C2CCC1CC(N)C2)C(F)(F)c1ccc(C(F)(F)F)cc1)S(=O)(=O)c1ccc2cc(OC3CCCC3)ccc2c1]

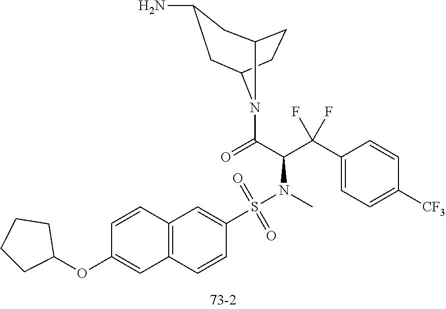 [SMILES: CN([C@@H](C(=O)N1C2CCC1CC(N)C2)C(F)(F)c1ccc(C(F)(F)F)cc1)S(=O)(=O)c1ccc2cc(OC3CCCC3)ccc2c1]

Compounds 73A and 73 were synthesized by employing the procedures described for Compounds 34A and 6 using Compounds 54I and 73A in lieu of Compounds 5A and 6C.

Compound 73A: LC-MS (ESI) m/z: 666 [M−Boc+H]⁺.

Compound 73 was separated with chiral HPLC to give Compound 73-1 and Compound 73-2.

Compound 73-1: LC-MS (ESI) m/z: 666 [M+H]⁺; ¹H-NMR (CD₃OD, 400 MHz): δ (ppm) 0.86-1.23 (m, 2H), 1.36-1.38 (m, 1H), 1.53-1.94 (m, 12H), 2.01-2.11 (m, 2H), 3.07-3.26 (m, 4H), 4.38-4.66 (m, 1H), 5.00-5.03 (m, 1H), 5.50-5.73 (m, 1H), 7.24-7.31 (m, 2H), 7.50-7.63 (m, 1H), 7.69-7.98 (m, 6H), 8.25-8.34 (m, 1H). Chiral separation condition: MeOH contained 0.5% NH₄OH; (R, R)-Whelk-O1 (4.6×250 mm, 5 μm); retention time: 4.42 minute.

Compound 73-2: LC-MS (ESI) m/z: 666 [M+H]⁺; ¹H-NMR (CD₃OD, 400 MHz): δ (ppm) 0.86-0.93 (m, 1H), 1.36-1.38 (m, 1H), 1.70-1.94 (m, 10H), 2.03-2.24 (m, 5H), 3.03-3.23 (m, 3H), 3.62-3.73 (m, 1H), 4.61-4.72 (m, 1H), 5.00-5.04 (m, 1H), 5.59-5.69 (m, 1H), 7.24-7.36 (m, 2H), 7.58-7.96 (m, 7H), 8.19-8.33 (m, 1H). Chiral separation condition: MeOH contained 0.5% NH₄OH; (R, R)-Whelk-O1 (4.6×250 mm, 5 μm); retention time: 4.96 minute.

### Example 74

### Synthesis of N-((2R)-3-(3-amino-8-azabicyclo[3.2.1]octan-8-yl)-1-(4-bromophenyl)-1,1-difluoro-3-oxopropan-2-yl)-1-(cyclopentylmethyl)-1H-indazole-5-sulfonamide (Compound 74-1), and N-((2S)-3-(3-amino-8-azabicyclo[3.2.1]octan-8-yl)-1-(4-bromophenyl)-1,1-difluoro-3-oxopropan-2-yl)-1-(cyclopentylmethyl)-1H-indazole-5-sulfonamide (Compound 74-2)

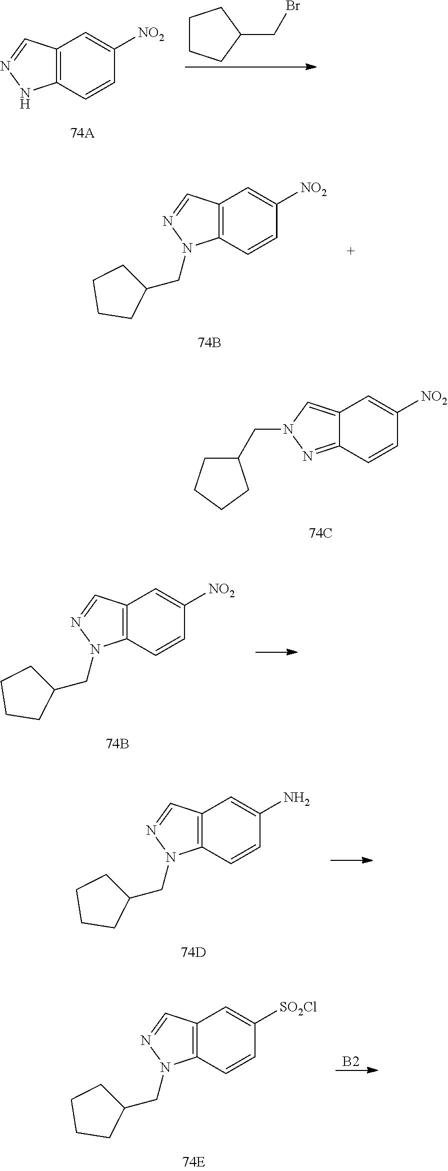

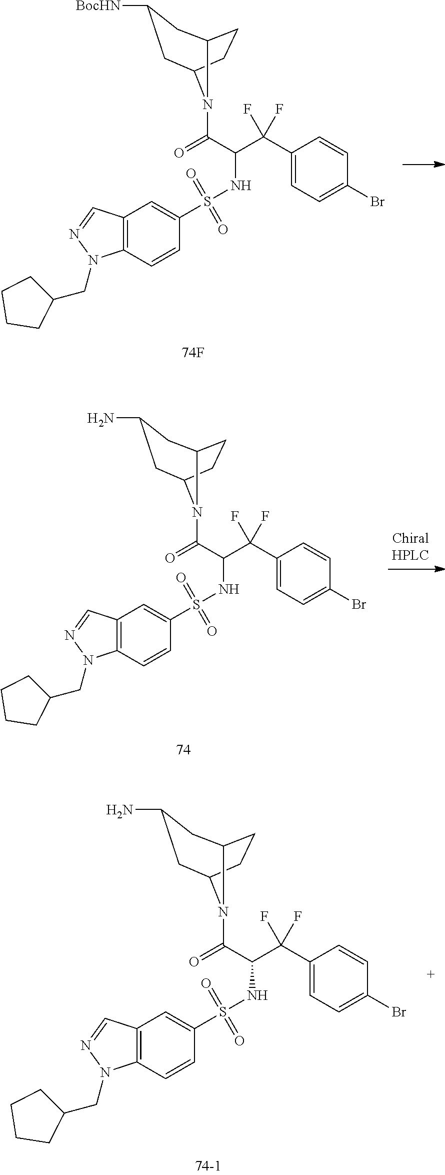 [SMILES: CC(C)(C)OC(=O)NC1CC2CCC(C1)N2C(=O)C(NS(=O)(=O)c1ccc2c(cnn2CC2CCCC2)c1)C(F)(F)c1ccc(Br)cc1 ; NC1CC2CCC(C1)N2C(=O)C(NS(=O)(=O)c1ccc2c(cnn2CC2CCCC2)c1)C(F)(F)c1ccc(Br)cc1 ; NC1CC2CCC(C1)N2C(=O)[C@@H](NS(=O)(=O)c1ccc2c(cnn2CC2CCCC2)c1)C(F)(F)c1ccc(Br)cc1]

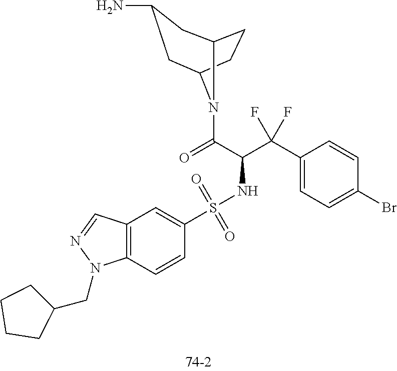 [SMILES: NC1CC2CCC(C1)N2C(=O)[C@H](NS(=O)(=O)c1ccc2c(cnn2CC2CCCC2)c1)C(F)(F)c1ccc(Br)cc1]

A mixture of Compound 74A (5 g, 30.6 mmol), (bromomethyl)cyclopentane (10 g, 61.3 mmol), and Cs₂CO₃ (30 g, 92 mmol) in DMF (100 mL) was stirred at room temperature for 18 hours. The reaction mixture was treated with H₂O (50 mL) and ethyl acetate (50 mL), washed with water (500 mL×3), dried over anhydrous sodium sulfate, filtered, and concentrated. The crude product was purified by silica gel column chromatography to furnish Compound 74B and Compound 74C.

Compound 74B: LC-MS (ESI) m/z: 246 [M+H]⁺; ¹H-NMR (CDCl₃, 400 MHz): δ (ppm) 1.30-1.31 (m, 2H), 1.57-1.60 (m, 2H), 1.58-1.78 (m, 4H), 2.55-2.59 (m, 1H), 4.39 (d, J=7.6 Hz, 2H), 7.75 (d, J=9.6 Hz, 1H), 8.10 (d, J=9.6 Hz, 1H), 8.21 (s, 1H), 8.73 (s, 1H).

Compound 74C: LC-MS (ESI) m/z: 246 [M+H]⁺; ¹H-NMR (CDCl₃, 400 MHz): δ (ppm) 0.80-0.89 (m, 2H), 1.26-1.36 (m, 2H), 1.60-1.78 (m, 4H), 2.63-2.65 (m, 1H), 4.39 (d, J=7.6 Hz, 2H), 7.75 (d, J=9.6 Hz, 1H), 8.10 (d, J=9.6 Hz, 1H), 8.21 (s, 1H), 8.73 (s, 1H).

To a solution of Compound 74B (4 g, 16.3 mmol) in methanol (100 mL) was added 10% palladium on carbon (0.8 g). The reaction mixture was stirred under 1 atm of hydrogen atmosphere at 23° C. for 2 days. The solvent was filtered and concentrated under reduced pressure to give Compound 74D as a solid. LC-MS (ESI) m/z: 216 [M+H]⁺; ¹H-NMR (CDCl₃, 400 MHz): δ (ppm) 1.26-1.34 (m, 2H), 1.53-1.57 (m, 2H), 1.57-1.68 (m, 4H), 2.49-2.54 (m, 1H), 3.48 (brs, 2H), 4.22 (d, J=7.6 Hz, 2H), 6.86 (dd, J=1.6, 8.8 Hz, 1H), 6.92 (d, J=1.6 Hz, 1H), 7.24 (d, J=8.8 Hz, 1H), 7.76 (s, 1H).

To a solution of Compound 74D (2 g, 9.25 mmol) in acetonitrile (60 mL) and acetic acid (9 mL) was added con. HCl (7.5 mL, 12M). To the resulting red mixture in an ice-water bath was added a solution of sodium nitrite (1.66 g, 24 mmol) in water (3 mL). After it was stirred at 0° C. for 1 hour, to the reaction mixture was added a solution of sulfur dioxide in acetic acid (20 mL), followed by addition of CuCl₂ (3.2 g, 24 mmol). The reaction mixture was stirred at 23° C. overnight, treated with H₂O (50 mL) and ethyl acetate (50 mL), washed with water (500 mL×3), dried over anhydrous sodium sulfate, filtered, and concentrated. The crude product was purified by silica gel column chromatography to furnish Compound 74E. LC-MS (ESI) m/z: 299 [M+H]⁺; ¹H-NMR (CDCl₃, 400 MHz): δ (ppm) 1.26-1.34 (m, 2H), 1.50-1.52 (m, 2H), 1.60-1.64 (m, 4H), 2.52-2.54 (m, 1H), 4.30 (d, J=7.6 Hz, 2H), 7.53 (d, J=9.2 Hz, 1H), 7.91 (d, J=9.2 Hz, 1H), 8.15 (s, 1H), 8.45 (s, 1H).

Compounds 74F and 74 were synthesized by employing the procedures described for Compounds 2D and 1 using Compounds 74E and 74F in lieu of Compounds 2C and 1A.

Compound 74F: LC-MS (ESI) m/z: 772 [M+Na]⁺.

Compound 74 was separated with chiral HPLC (Chiral separation condition: MeOH contained 0.1% DEA; IC, 250×4.6 mm, 5 m) to give Compound 74-1 and Compound 74-2.

Compound 74-1: LC-MS (ESI) m/z: 650 [M+H]⁺; ¹H-NMR (CD₃OD, 400 MHz): δ (ppm) 0.78-1.18 (m, 3H), 1.23-1.31 (m, 2H), 1.48-1.80 (m, 11H), 2.44-2.48 (m, 1H), 3.07-3.11 (m, 1H), 4.20-4.42 (m, 4H), 4.54-4.72 (m, 1H), 7.19-7.21 (m, 1H), 7.29-7.33 (m, 2H), 7.43-7.45 (m, 1H), 7.54-7.56 (m, 2H), 8.06-8.11 (m, 2H). Chiral separation condition: MeOH contained 0.1% DEA; IC (4.6×250 mm, 5 μm); retention time: 5.06 minute.

Compound 74-2: LC-MS (ESI) m/z: 650 [M+H]⁺; ¹H-NMR (CD₃OD, 400 MHz): δ (ppm) 0.70-1.18 (m, 2H), 1.23-1.30 (m, 3H), 1.45-1.81 (m, 11H), 2.42-2.48 (m, 1H), 3.05-3.08 (m, 1H), 4.19-4.40 (m, 4H), 4.54-4.70 (m, 1H), 7.19-7.21 (m, 1H), 7.30-7.33 (m, 2H), 7.43-7.45 (m, 1H), 7.55-7.56 (m, 2H), 8.06-8.11 (m, 2H). Chiral separation condition: MeOH contained 0.1% DEA; IC (4.6×250 mm, 5 μm); retention time: 4.17 minute.

### Example 75

### Synthesis of N-((2R)-3-(3-amino-8-azabicyclo[3.2.1]octan-8-yl)-1-(4-bromophenyl)-1,1-difluoro-3-oxopropan-2-yl)-1-(cyclopentylmethyl)-N-methyl-1H-indazole-5-sulfonamide (Compound 75-1), and N-((2S)-3-(3-amino-8-azabicyclo[3.2.1]octan-8-yl)-1-(4-bromophenyl)-1,1-difluoro-3-oxopropan-2-yl)-1-(cyclopentylmethyl)-N-methyl-1H-indazole-5-sulfonamide (Compound 75-2)

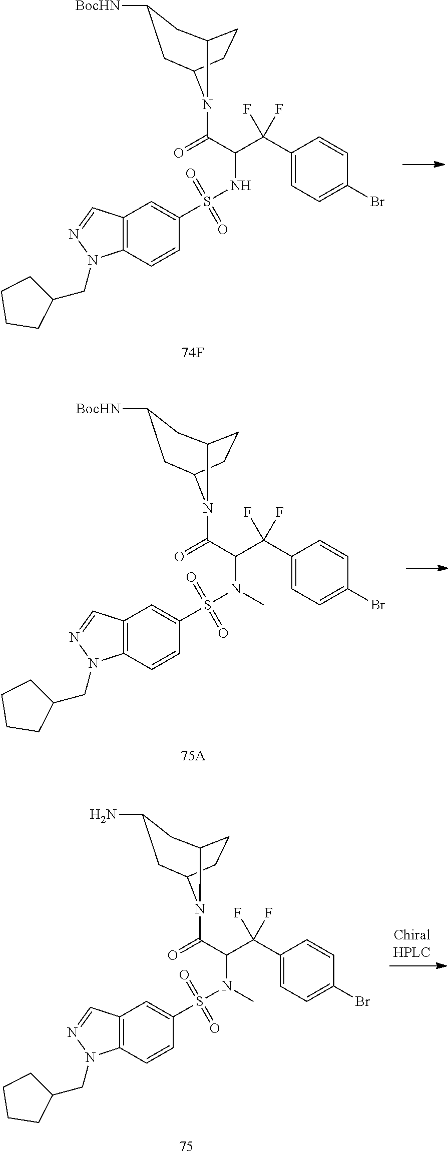 [SMILES: CC(C)(C)OC(=O)NC1CC2CCC(C1)N2C(=O)C(NS(=O)(=O)c1ccc2c(cnn2CC2CCCC2)c1)C(F)(F)c1ccc(Br)cc1 ; CN(C(C(=O)N1C2CCC1CC(NC(=O)OC(C)(C)C)C2)C(F)(F)c1ccc(Br)cc1)S(=O)(=O)c1ccc2c(cnn2CC2CCCC2)c1 ; CN(C(C(=O)N1C2CCC1CC(N)C2)C(F)(F)c1ccc(Br)cc1)S(=O)(=O)c1ccc2c(cnn2CC2CCCC2)c1]

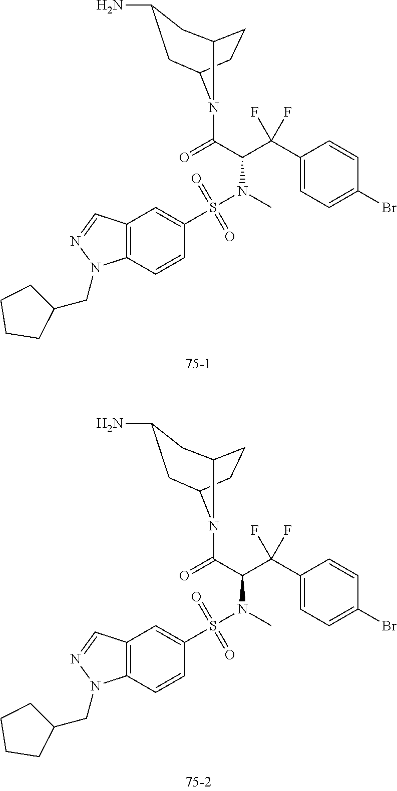 [SMILES: CN([C@H](C(=O)N1C2CCC1CC(N)C2)C(F)(F)c1ccc(Br)cc1)S(=O)(=O)c1ccc2c(cnn2CC2CCCC2)c1 ; CN([C@@H](C(=O)N1C2CCC1CC(N)C2)C(F)(F)c1ccc(Br)cc1)S(=O)(=O)c1ccc2c(cnn2CC2CCCC2)c1]

Compounds 75A and 75 were synthesized by employing the procedures described for Compounds 34A and 1 using Compounds 74F and 75A in lieu of Compounds 5A and 1A.

Compound 75A was used for the next step without any purification. LC-MS (ESI) m/z: 786 [M+Na]⁺.

Compound 75 was separated with chiral HPLC (Chiral separation condition: MeOH contained 0.1% DEA; OD 250×4.6 mm, 5 m) to give Compound 75-1 and Compound 75-2.

Compound 75-1: LC-MS (ESI) m/z: 664 [M+H]⁺; ¹H-NMR (CD₃OD, 400 MHz): δ (ppm) 0.72-1.17 (m, 2H), 1.23-1.86 (m, 14H), 2.46-2.47 (m, 1H), 2.96-3.21 (m, 4H), 4.30-4.47 (m, 4H), 5.37-5.49 (m, 1H), 7.26-7.28 (m, 1H), 7.35-7.37 (m, 1H), 7.42-7.56 (m, 3H), 7.61-7.64 (m, 1H), 8.12-8.18 (m, 2H). Chiral separation condition: MeOH contained 0.1% DEA; OD (4.6×250 mm, 5 μm); retention time: 5.52 minute.

Compound 75-2: LC-MS (ESI) m/z: 664 [M+H]⁺; ¹H-NMR (CD₃OD, 400 MHz): δ (ppm) 0.72-1.10 (m, 2H), 1.10-1.17 (m, 2H), 1.23-1.86 (m, 12H), 2.44-2.49 (m, 1H), 2.94-3.09 (m, 4H), 4.28-4.50 (m, 4H), 5.35-5.49 (m, 1H), 7.26-7.28 (m, 1H), 7.34-7.37 (m, 1H), 7.42-7.44 (m, 1H), 7.45-7.56 (m, 3H), 8.12-8.18 (m, 2H). Chiral separation condition: MeOH contained 0.1% DEA; OD (4.6×250 mm, 5 μm); retention time: 12.27 minute.

### Example 76

### Synthesis of N-((2R)-3-(3-amino-8-azabicyclo[3.2.1]octan-8-yl)-1-(4-bromophenyl)-1,1-difluoro-3-oxopropan-2-yl)-6-(cyclopentyloxy)-N-isopropylnaphthalene-2-sulfonamide (Compound 76-1), and N-((2S)-3-(3-amino-8-azabicyclo[3.2.1]octan-8-yl)-1-(4-bromophenyl)-1,1-difluoro-3-oxopropan-2-yl)-6-(cyclopentyloxy)-N-isopropylnaphthalene-2-sulfonamide (Compound 76-2)

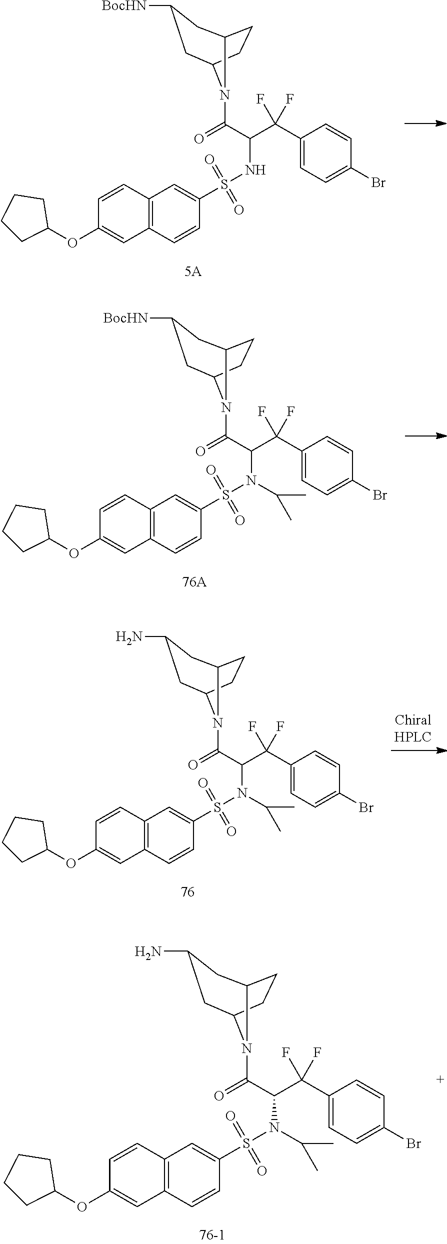 [SMILES: CC(C)(C)OC(=O)NC1CC2CCC(C1)N2C(=O)C(NS(=O)(=O)c1ccc2cc(OC3CCCC3)ccc2c1)C(F)(F)c1ccc(Br)cc1 ; CC(C)N(C(C(=O)N1C2CCC1CC(NC(=O)OC(C)(C)C)C2)C(F)(F)c1ccc(Br)cc1)S(=O)(=O)c1ccc2cc(OC3CCCC3)ccc2c1 ; CC(C)N(C(C(=O)N1C2CCC1CC(N)C2)C(F)(F)c1ccc(Br)cc1)S(=O)(=O)c1ccc2cc(OC3CCCC3)ccc2c1 ; CC(C)N([C@H](C(=O)N1C2CCC1CC(N)C2)C(F)(F)c1ccc(Br)cc1)S(=O)(=O)c1ccc2cc(OC3CCCC3)ccc2c1]

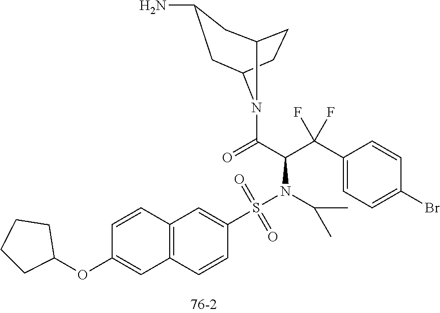 [SMILES: CC(C)N([C@@H](C(=O)N1C2CCC1CC(N)C2)C(F)(F)c1ccc(Br)cc1)S(=O)(=O)c1ccc2cc(OC3CCCC3)ccc2c1]

A mixture of Compound 5A (100 mg, 0.13 mmol), Cs₂CO₃ (130 mg, 0.4 mmol), and 2-iodopropane (45 mg, 0.26 mmol) in NMP (5 mL) was stirred at 60° C. for 72 hours. The reaction mixture was cooled down to room temperature, diluted with water (50 mL), and extracted with ethyl acetate (50 mL×2). The combined organic extracts was washed with water (20 mL×3) and brine (50 mL), dried over anhydrous sodium sulfate, filtered, and concentrated to afford Compound 76A. LC-MS (ESI) m/z: 826 [M+Na]⁺.

Compound 76 was synthesized by employing the procedure described for Compound 1 using Compound 76A in lieu of Compound 1A, which was separated with chiral-HPLC to furnish Compound 76-1 and Compound 76-2.

Compound 76-1: LC-MS (ESI) m/z: 704 [M+H]⁺; ¹H-NMR (CD₃OD, 400 MHz): δ (ppm) 0.29-1.15 (m, 3H), 1.39-1.59 (m, 8H), 1.70-1.90 (m, 9H), 2.06-2.07 (m, 2H), 3.06-3.13 (m, 1H), 3.57-3.65 (m, 1H), 4.14-4.52 (m, 1H), 4.60-4.72 (m, 1H), 5.02-5.12 (m, 2H), 7.15-7.33 (m, 4H), 7.48-7.52 (m, 2H), 7.71-7.99 (m, 3H), 8.37-8.42 (m, 1H). Chiral separation condition: n-hexane/EtOH contained 0.1% DEA, 70/30; AY-H (4.6×250 mm, 5 m; retention time: 12.78 minute.

Compound 76-2: LC-MS (ESI) m/z: 704 [M+H]⁺; ¹H-NMR (CD₃OD, 400 MHz): δ (ppm) 0.23-1.28 (m, 3H), 1.37-1.57 (m, 8H), 1.70-1.92 (m, 9H), 2.01-2.06 (m, 2H), 3.04-3.07 (m, 1H), 3.56-3.64 (m, 1H), 4.12-4.50 (m, 1H), 4.60-4.71 (m, 1H), 5.02-5.17 (m, 2H), 7.17-7.32 (m, 4H), 7.47-7.52 (m, 2H), 7.71-7.99 (m, 3H), 8.37-8.42 (m, 1H). Chiral separation condition: n-hexane/EtOH contained 0.1% DEA, 70/30; AY-H (4.6×250 mm, 5 m; retention time: 7.05 minute.

### Example 77

### Synthesis of N-((2R)-3-(3-amino-8-azabicyclo[3.2.1]octan-8-yl)-1-(4-bromophenyl)-1,1-difluoro-3-oxopropan-2-yl)-2-(cyclopentylmethyl)-2H-indazole-5-sulfonamide (Compound 77-1), and N-((2S)-3-(3-amino-8-azabicyclo[3.2.1]octan-8-yl)-1-(4-bromophenyl)-1,1-difluoro-3-oxopropan-2-yl)-2-(cyclopentylmethyl)-2H-indazole-5-sulfonamide (Compound 77-2)

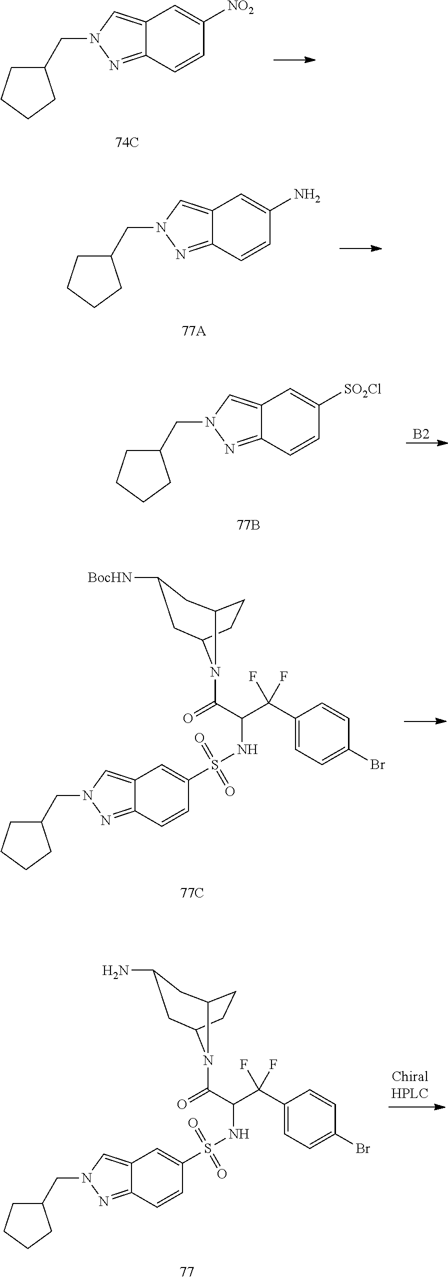 [SMILES: O=[N+]([O-])c1ccc2nn(CC3CCCC3)cc2c1 ; Nc1ccc2nn(CC3CCCC3)cc2c1 ; O=S(=O)(Cl)c1ccc2nn(CC3CCCC3)cc2c1 ; "B2" ; CC(C)(C)OC(=O)NC1CC2CCC(C1)N2C(=O)C(NS(=O)(=O)c1ccc2nn(CC3CCCC3)cc2c1)C(F)(F)c1ccc(Br)cc1 ; NC1CC2CCC(C1)N2C(=O)C(NS(=O)(=O)c1ccc2nn(CC3CCCC3)cc2c1)C(F)(F)c1ccc(Br)cc1]

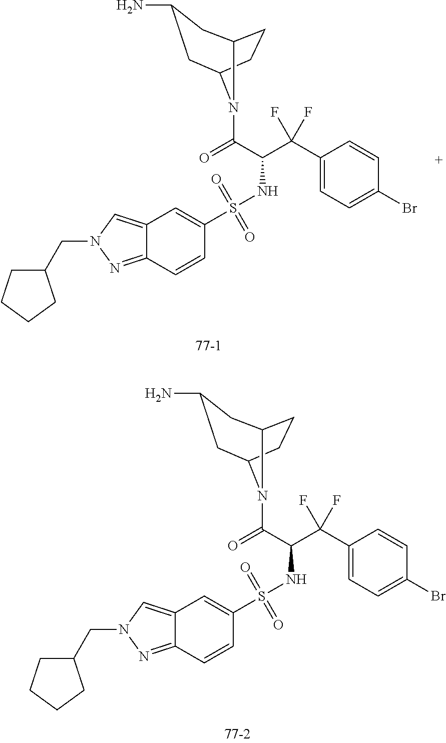 [SMILES: NC1CC2CCC(C1)N2C(=O)[C@@H](NS(=O)(=O)c1ccc2nn(CC3CCCC3)cc2c1)C(F)(F)c1ccc(Br)cc1 ; NC1CC2CCC(C1)N2C(=O)[C@H](NS(=O)(=O)c1ccc2nn(CC3CCCC3)cc2c1)C(F)(F)c1ccc(Br)cc1]

Compounds 77A, 77B, 77C, and 77 were synthesized by employing the procedures described for Compounds 74D, 74E, 2D, and 1 using Compounds 74C, 77A, 77B, and 77C in lieu of Compounds 74B, 74D, 2C, and 1A.

Compound 77A: LC-MS (ESI) m/z: 216 [M+H]⁺.

Compound 77B: LC-MS (ESI) m/z: 299 [M+H]⁺.

Compound 77C: LC-MS (ESI) m/z: 772 [M+Na]⁺.

Compound 77 was separated with chiral HPLC (Chiral separation condition: MeOH contained 0.5% NH₄OH; OJ, 250×4.6 mm 5 m) to give Compound 77-1 and Compound 77-2.

Compound 77-1: LC-MS (ESI) m/z: 650 [M+H]⁺; ¹H-NMR (CD₃OD, 400 MHz): δ (ppm) 0.67-1.18 (m, 4H), 1.23-1.61 (m, 12H), 2.44-2.53 (m, 1H), 2.73-2.97 (m, 1H), 4.12-4.34 (m, 4H), 4.51-4.62 (m, 1H), 7.23-7.57 (m, 6H), 8.10-8.12 (m, 1H), 8.36-8.38 (m, 1H). Chiral separation condition: MeOH contained 0.5% NH₄OH; OJ (4.6×250 mm 5 μm); retention time: 6.66 minute.

Compound 77-2: LC-MS (ESI) m/z: 650 [M+H]⁺; ¹H-NMR (CD₃OD, 400 MHz): δ (ppm) 0.65-1.16 (m, 3H), 1.26-1.71 (m, 13H), 2.49-2.54 (m, 1H), 2.82-2.92 (m, 1H), 4.14-4.34 (m, 4H), 4.51-4.61 (m, 1H), 7.23-7.57 (m, 6H), 8.11-8.13 (m, 1H), 8.37-8.38 (m, 1H). Chiral separation condition: MeOH contained 0.5% NH₄OH; OJ (4.6×250 mm 5 μm); retention time: 4.66 minute.

### Example 78

### Synthesis of N-((2R)-3-(3-amino-8-azabicyclo[3.2.1]octan-8-yl)-1-(4-bromophenyl)-1,1-difluoro-3-oxopropan-2-yl)-2-(cyclopentylmethyl)-N-methyl-2H-indazole-5-sulfonamide (Compound 78-1), and N-((2S)-3-(3-amino-8-azabicyclo[3.2.1]octan-8-yl)-1-(4-bromophenyl)-1,1-difluoro-3-oxopropan-2-yl)-2-(cyclopentylmethyl)-N-methyl-2H-indazole-5-sulfonamide (Compound 78-2)

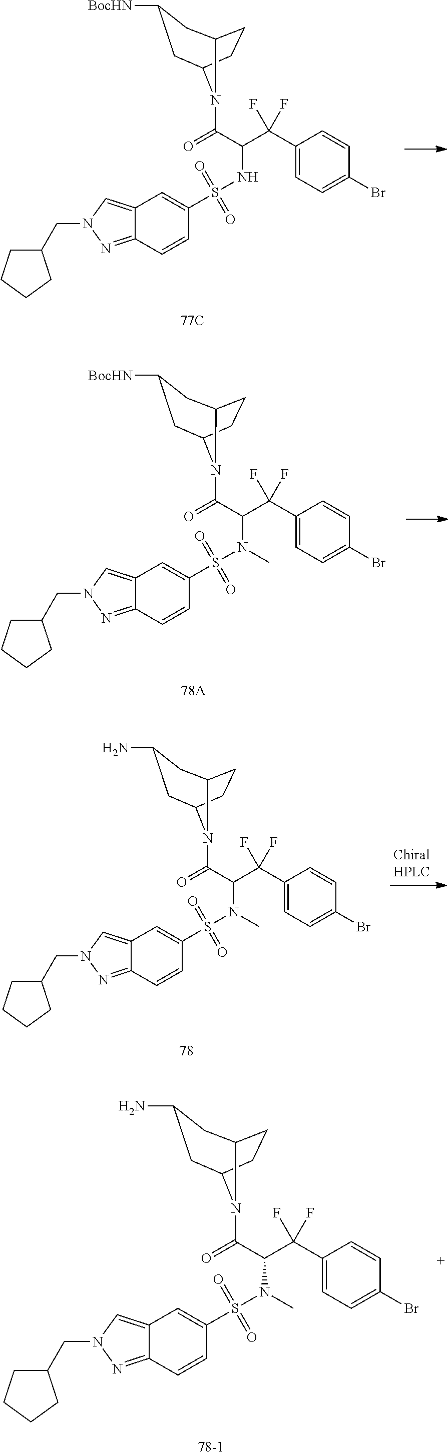 [SMILES: CC(C)(C)OC(=O)NC1CC2CCC(C1)N2C(=O)C(NS(=O)(=O)c1ccc2nn(CC3CCCC3)cc2c1)C(F)(F)c1ccc(Br)cc1 ; CN(C(C(=O)N1C2CCC1CC(NC(=O)OC(C)(C)C)C2)C(F)(F)c1ccc(Br)cc1)S(=O)(=O)c1ccc2nn(CC3CCCC3)cc2c1 ; CN(C(C(=O)N1C2CCC1CC(N)C2)C(F)(F)c1ccc(Br)cc1)S(=O)(=O)c1ccc2nn(CC3CCCC3)cc2c1 ; CN([C@H](C(=O)N1C2CCC1CC(N)C2)C(F)(F)c1ccc(Br)cc1)S(=O)(=O)c1ccc2nn(CC3CCCC3)cc2c1]

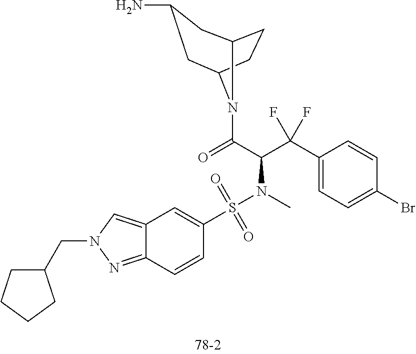 [SMILES: CN([C@@H](C(=O)N1C2CCC1CC(N)C2)C(F)(F)c1ccc(Br)cc1)S(=O)(=O)c1ccc2nn(CC3CCCC3)cc2c1]

Compounds 78A and 78 were synthesized by employing the procedures described for Compounds 34A and 1 using Compounds 77C and 78A in lieu of Compounds 5A and 1A.

Compound 78A was used for the next step without further purification. LC-MS (ESI) m/z: 786 [M+Na]⁺.

Compound 78 was separated with chiral HPLC (Chiral separation condition: MeOH contained 0.5% NH₄OH; OJ, 250×4.6 mm 5 m) to give Compound 78-1 and Compound 78-2.

Compound 78-1: LC-MS (ESI) m/z: 664 [M+H]⁺; ¹H-NMR (CD₃OD, 400 MHz): δ (ppm) 0.82-1.21 (m, 1H), 1.36-2.01 (m, 15H), 2.62-2.64 (m, 1H), 3.06-3.20 (m, 4H), 4.43-4.64 (m, 4H), 5.40-5.59 (m, 1H), 7.33-7.71 (m, 6H), 8.25-8.28 (m, 1H), 8.52-8.53 (m, 1H). Chiral separation condition: MeOH contained 0.5% NH₄OH; OJ (4.6×250 mm 5 μm); retention time: 11.64 minute.

Compound 78-2: LC-MS (ESI) m/z: 664 [M+H]⁺; ¹H-NMR (CD₃OD, 400 MHz): δ (ppm) 0.72-1.20 (m, 1H), 1.31-1.96 (m, 15H), 2.57-2.67 (m, 1H), 3.07-3.20 (m, 4H), 4.34-4.62 (m, 4H), 5.40-5.60 (m, 1H), 7.36-7.72 (m, 6H), 7.27-7.29 (m, 1H), 7.53-7.54 (m, 1H). Chiral separation condition: MeOH contained 0.5% NH₄OH; OJ (4.6×250 mm 5 m); retention time: 9.64 minute.

### Example 79

### Synthesis of N-((2R)-3-(3-amino-8-azabicyclo[3.2.1]octan-8-yl)-1-(4-butoxyphenyl)-1,1-difluoro-3-oxopropan-2-yl)-4-(cyclohexylmethoxy)-N-methylbenzenesulfonamide (Compound 79-1), and N-((2S)-3-(3-amino-8-azabicyclo[3.2.1]octan-8-yl)-1-(4-butoxyphenyl)-1,1-difluoro-3-oxopropan-2-yl)-4-(cyclohexylmethoxy)-N-methylbenzenesulfonamide (Compound 79-2)

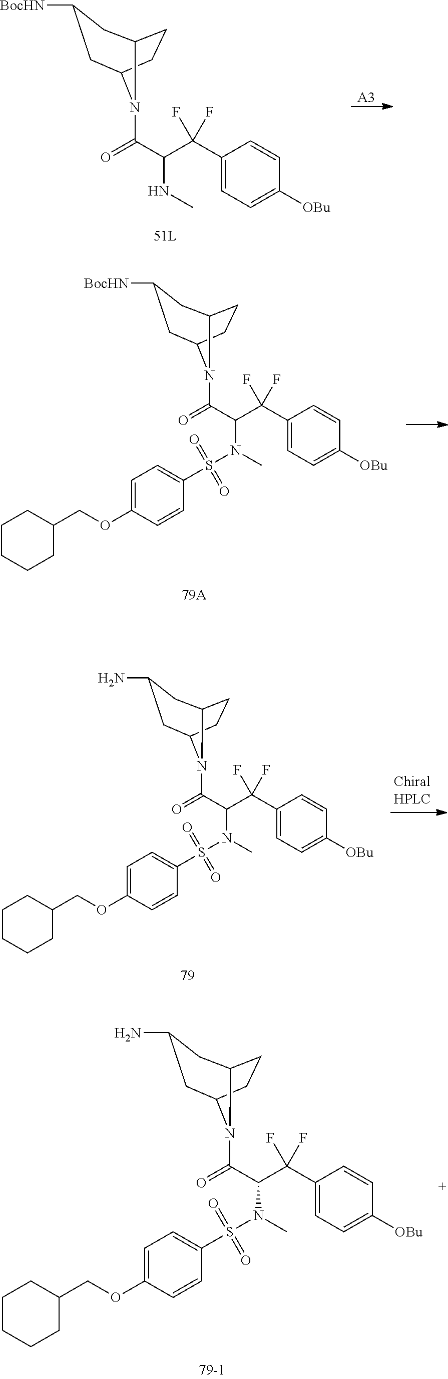

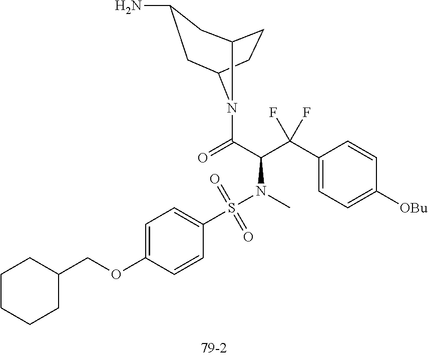

Compounds 79A and 79 were synthesized by employing the procedures described for Compounds 1A and 6 using Compound A3, Compounds 51L, and 79A in lieu of Compounds A1, B2, and Compound 6C.

Compound 79A: LC-MS (ESI) m/z: 648 [M−Boc+H]⁺.

Compound 79 was separated with chiral HPLC to give Compound 79-1 and Compound 79-2.

Compound 79-1: LC-MS (ESI) m/z: 648 [M+H]⁺; ¹H-NMR (CD₃OD, 400 MHz): δ (ppm) 0.74-0.82 (m, 1H), 0.90 (t, J=7.2 Hz, 3H), 0.98-1.06 (m, 2H), 1.12-1.32 (m, 4H), 1.37-1.94 (m, 16H), 2.90-3.08 (m, 4H), 3.76-3.77 (m, 2H), 3.90-3.93 (m, 2H), 4.09-4.46 (m, 2H), 5.20-5.39 (m, 1H), 6.83-6.93 (m, 4H), 7.23-7.34 (m, 2H), 7.50-7.55 (m, 2H); Chiral separation condition: MeOH contained 0.1% NH₄OH; (R,R)-Whelk-O1 (4.6×250 mm, 5 μm); retention time: 7.51 minute.

Compound 79-2: LC-MS (ESI) m/z: 648 [M+H]⁺; ¹H-NMR (CD₃OD, 400 MHz): δ (ppm) 1.01 (t, J=7.6 Hz, 3H), 1.09-1.17 (m, 2H), 1.24-1.37 (m, 5H), 1.50-2.07 (m, 17H), 3.04-3.18 (m, 3H), 3.80-3.89 (m, 2H), 4.01-4.04 (m, 2H), 4.26-4.60 (m, 2H), 5.33-5.49 (m, 1H), 6.92-7.04 (m, 4H), 7.33-7.75 (m, 4H); Chiral separation condition: MeOH contained 0.1% NH₄OH; (R,R)-Whelk-O1 (4.6×250 mm, 5 μm); retention time: 8.77 min.

### Example 80

### Synthesis of (R)—N-(3-(4-aminopiperidin-1-yl)-1-(4-butoxyphenyl)-1,1-difluoro-3-oxopropan-2-yl)-6-(cyclopentyloxy)naphthalene-2-sulfonamide (Compound 80-1), and (S)—N-(3-(4-aminopiperidin-1-yl)-1-(4-butoxyphenyl)-1,1-difluoro-3-oxopropan-2-yl)-6-(cyclopentyloxy)naphthalene-2-sulfonamide (Compound 80-2)

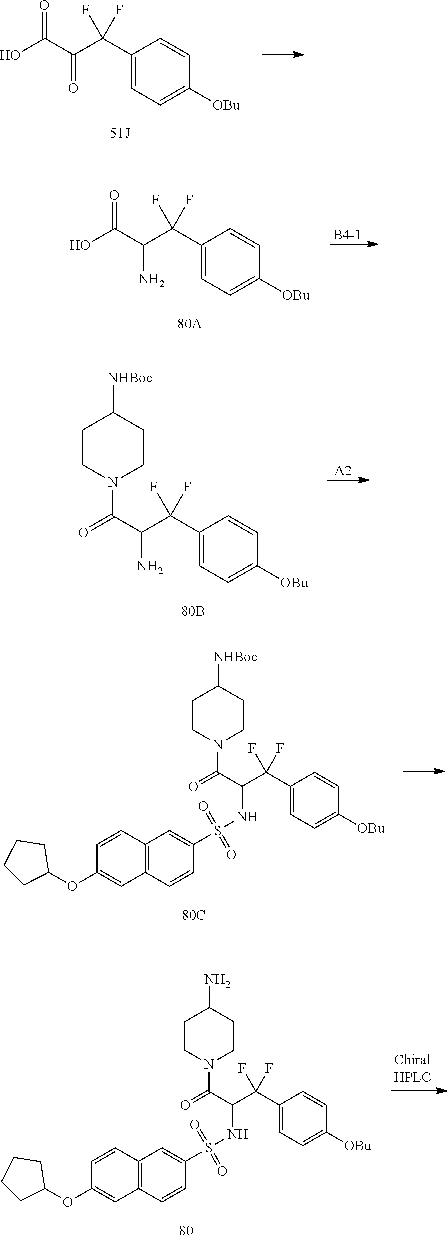

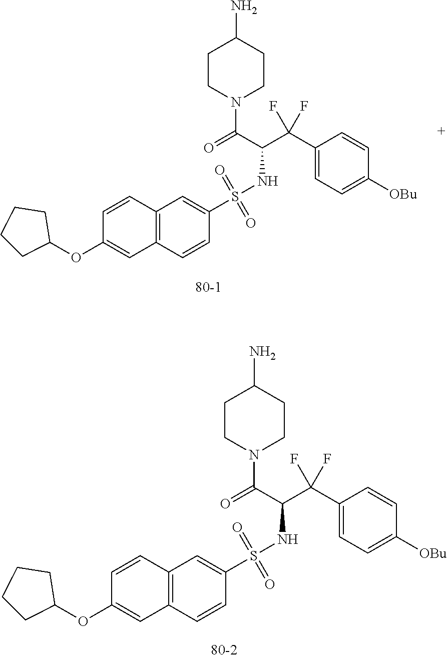

A mixture of Compound 51J (500 mg, 1.84 mmol) and hydroxylamine hydrochloride (153 mg, 2.2 mmol) in ethanol (10 mL) was stirred at 40° C. overnight. After the reaction mixture was cooled down, to it was added zinc powder (1.2 g, 18.4 mmol), water (2 mL), and formic acid (4 mL) and stirred at room temperature for 2 hours. The reaction mixture was filtered and concentrated under reduced pressure. The residue was purified with reverse phase chromatography using eluents (methanol in water, from 0% to 100% v/v) to furnish Compound 80A. LC-MS (ESI) m/z: 272 [M−H]⁺; ¹H-NMR (DMSO-d₆, 400 MHz): δ (ppm) 0.90-096 (m, 3H), 1.39-1.47 (m, 2H), 1.65-1.73 (m, 2H), 3.90-4.09 (m, 3H), 6.80-6.82 (m, 1H), 7.03-7.13 (m, 2H), 7.47-7.49 (m, 1H).

Compounds 80B, 80C, and 80 were synthesized by employing the procedures described for Compound B4, Compounds 1A, and 6 using Compounds 80A, 80B, Compound A2, and 80C in lieu of Compounds B2-6, B2, A1, and Compound 6C.

Compound 80B: LC-MS (ESI) m/z: 456 [M+H]⁺.

Compound 80C: LC-MS (ESI) m/z: 674 [M−55]⁺.

Compound 80 was separated with chiral HPLC to give Compound 80-1 and Compound 80-2.

Compound 80-1: LC-MS (ESI) m/z: 630 [M+H]⁺; ¹H-NMR (CD₃OD, 400 MHz): δ (ppm) 0.23-0.55 (m, 1H), 0.90-1.01 (m, 4H), 1.47-1.53 (m, 3H), 1.70-1.76 (m, 4H), 1.82-1.93 (m, 4H), 2.03-2.08 (m, 2H), 2.36-2.80 (m, 2H), 3.56-3.66 (m, 3H), 3.75-4.13 (m, 4H), 4.98-5.03 (m, 1H), 6.80-6.84 (m, 2H), 7.20-7.35 (m, 4H), 7.60-7.66 (m, 1H), 7.78-7.89 (m, 2H), 8.15-8.20 (m, 1H). Chiral separation condition: n-hexane/EtOH contained 0.1% DEA, 60/40; S,S-Whelk-O1 (4.6×250 mm, 5 μm); retention time: 15.71 minute.

Compound 80-2: LC-MS (ESI) m/z: 630 [M+H]⁺; ¹H-NMR (CD₃OD, 400 MHz): δ (ppm) 0.25-0.60 (m, 1H), 0.98-1.02 (m, 4H), 1.48-1.55 (m, 3H), 1.69-1.77 (m, 4H), 1.82-1.94 (m, 4H), 2.03-2.11 (m, 2H), 2.37-2.83 (m, 2H), 3.56-3.68 (m, 3H), 3.76-4.14 (m, 4H), 4.99-5.01 (m, 1H), 6.81-6.84 (m, 2H), 7.20-7.36 (m, 4H), 7.60-7.67 (m, 1H), 7.78-7.90 (m, 2H), 8.16-8.20 (m, 1H). Chiral separation condition: n-hexane/EtOH contained 0.1% DEA, 60/40; S,S-Whelk-O1 (4.6×250 mm, 5 μm); retention time: 12.16 minute.

### Example 81

### Synthesis of (R)—N-(3-(4-aminopiperidin-1-yl)-1-(4-butoxyphenyl)-1,1-difluoro-3-oxopropan-2-yl)-4-(cyclohexylmethoxy)benzenesulfonamide trifluoroacetate (Compound 81-1), and (S)—N-(3-(4-aminopiperidin-1-yl)-1-(4-butoxyphenyl)-1,1-difluoro-3-oxopropan-2-yl)-4-(cyclohexylmethoxy)benzenesulfonamide trifluoroacetate (Compound 81-2)

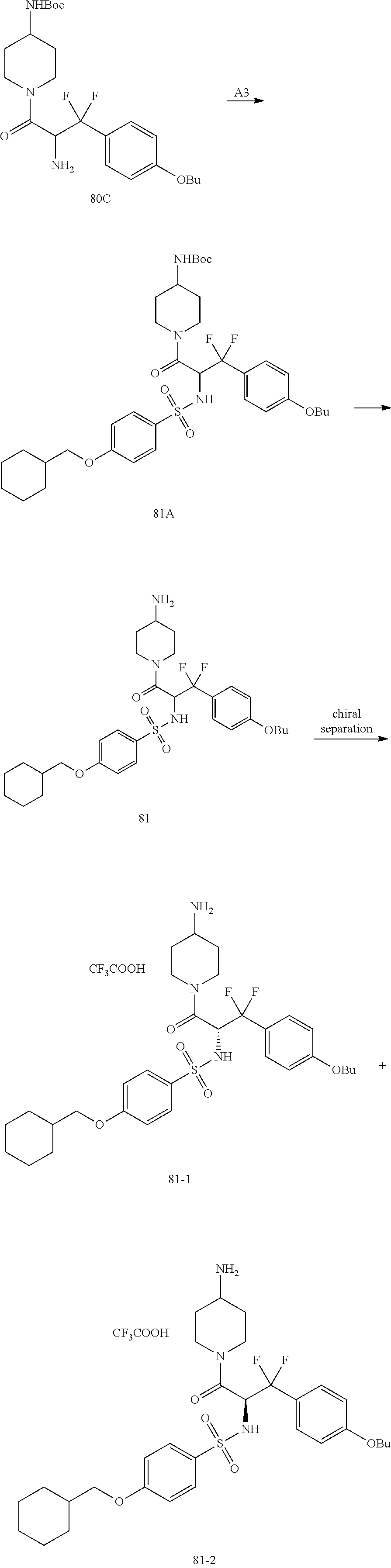

Compounds 81A and 81 were synthesized by employing the procedures described for Compounds 1A and 6 using Compound 80C, Compound A3, and Compound 81A in lieu of Compounds B2, A1, and Compound 6C.

Compound 81A: LC-MS (ESI) m/z: 652 [M−55]⁺.

Compound 81 was separated with chiral HPLC to give Compound 81-1 and Compound 81-2.

Compound 81-1: LC-MS (ESI) m/z: 608 [M+H]⁺; ¹H-NMR (CD₃OD, 400 MHz): δ (ppm) 1.02 (t, J=7.2 Hz, 3H), 1.08-1.17 (m, 2H), 1.25-2.11 (m, 17H), 2.42-2.75 (m, 1H), 3.01-3.14 (m, 1H), 3.35-3.41 (m, 1H), 3.84 (d, J=6.0 Hz, 2H), 3.98-4.04 (m, 2H), 4.12-4.20 (m, 1H), 4.34-4.55 (m, 1H), 4.84-4.95 (m, 1H), 6.81-6.93 (m, 4H), 7.26-7.38 (m, 2H), 7.50-7.56 (m, 2H). Chiral separation condition: n-hexane/EtOH contained 0.1% DEA, 60/40; S,S-Whelk-O1 (4.6×250 mm, 5 μm); retention time: 10.96 minute.

Compound 81-2: LC-MS (ESI) m/z: 608 [M+H]⁺; ¹H-NMR (CD₃OD, 400 MHz): δ (ppm) 1.02 (t, J=7.2 Hz, 3H), 1.08-1.17 (m, 2H), 1.24-2.11 (m, 17H), 2.42-2.75 (m, 1H), 3.01-3.15 (m, 1H), 3.35-3.42 (m, 1H), 3.84 (d, J=6.0 Hz, 2H), 3.98-4.04 (m, 2H), 4.12-4.20 (m, 1H), 4.34-4.55 (m, 1H), 4.84-4.86 (m, 1H), 6.81-6.93 (m, 4H), 7.26-7.38 (m, 2H), 7.50-7.57 (m, 2H). Chiral separation condition: n-hexane/EtOH contained 0.1% DEA, 60/40; S,S-Whelk-O1 (4.6×250 mm, 5 μm); retention time: 9.59 minute.

### Example 82

### Synthesis of N-((2R)-3-(3-amino-8-azabicyclo[3.2.1]octan-8-yl)-1-(4-butoxyphenyl)-1,1-difluoro-3-oxopropan-2-yl)-6-(cyclopentyloxy)naphthalene-2-sulfonamide trifluoroacetate (Compound 82-1), and N-((2S)-3-(3-amino-8-azabicyclo[3.2.1]octan-8-yl)-1-(4-butoxyphenyl)-1,1-difluoro-3-oxopropan-2-yl)-6-(cyclopentyloxy)naphthalene-2-sulfonamide trifluoroacetate (Compound 82-2)

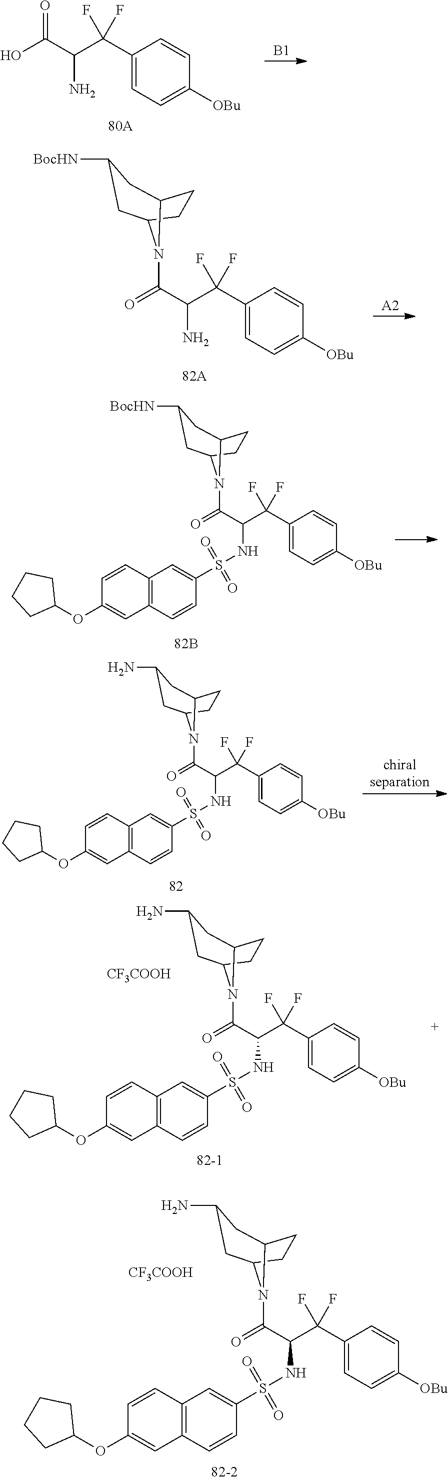

Compounds 82A, 82B, and 82 were synthesized by employing the procedures described for Compound B4, Compounds 1A, and 6 using Compound 80A, Compounds B1, A2, Compounds 82A, and 82B in lieu of Compounds B2-6, B4-1, A1, B2, and Compound 6C.

Compound 82A: LC-MS (ESI) m/z: 482 [M+H]⁺.

Compound 82B: LC-MS (ESI) m/z: 700 [M−55]⁺.

Compound 82 was separated with chiral HPLC to give Compound 82-1 and Compound 82-2.

Compound 82-1: LC-MS (ESI) m/z: 656 [M+H]⁺; ¹H-NMR (CD₃OD, 400 MHz): δ (ppm) 0.71-0.80 (m, 1H), 0.87-0.92 (m, 3H), 1.27-2.08 (m, 20H), 3.40-3.80 (m, 3H), 4.27-4.60 (m, 2H), 4.88-4.92 (m, 1H), 6.44-6.46 (m, 1H), 6.69-6.71 (m, 1H), 7.06-7.17 (m, 3H), 7.26-7.28 (m, 1H), 7.44-7.50 (m, 1H), 7.62-7.74 (m, 2H), 7.92-8.02 (m, 1H). Chiral separation condition: n-hexane/EtOH contained 0.1% DEA, 40/60; S,S-Whelk-O1 (4.6×250 mm, 5 μm); retention time: 8.83 minute.

Compound 82-2: LC-MS (ESI) m/z: 656 [M+H]⁺; ¹H-NMR (CD₃OD, 400 MHz): δ (ppm) 0.75-0.87 (m, 1H), 0.92-0.98 (m, 3H), 1.33-2.14 (m, 20H), 3.47-3.86 (m, 3H), 4.49-4.66 (m, 2H), 4.93-4.98 (m, 1H), 6.49-6.51 (m, 1H), 6.75-6.77 (m, 1H), 7.11-7.22 (m, 3H), 7.32-7.34 (m, 1H), 7.50-7.55 (m, 1H), 7.67-7.78 (m, 2H), 7.98-8.08 (m, 1H). Chiral separation condition: n-hexane/EtOH contained 0.1% DEA, 40/60; S,S-Whelk-O1 (4.6×250 mm, 5 μm); retention time: 7.66 minute.

### Example 83

### Synthesis of (R)—N-(3-(4-aminopiperidin-1-yl)-1-(3-butoxyphenyl)-1,1-difluoro-3-oxopropan-2-yl)-6-(cyclopentyloxy)naphthalene-2-sulfonamide trifluoroacetate (Compound 83-1), and (S)—N-(3-(4-aminopiperidin-1-yl)-1-(3-butoxyphenyl)-1,1-difluoro-3-oxopropan-2-yl)-6-(cyclopentyloxy)naphthalene-2-sulfonamide trifluoroacetate (Compound 83-2)

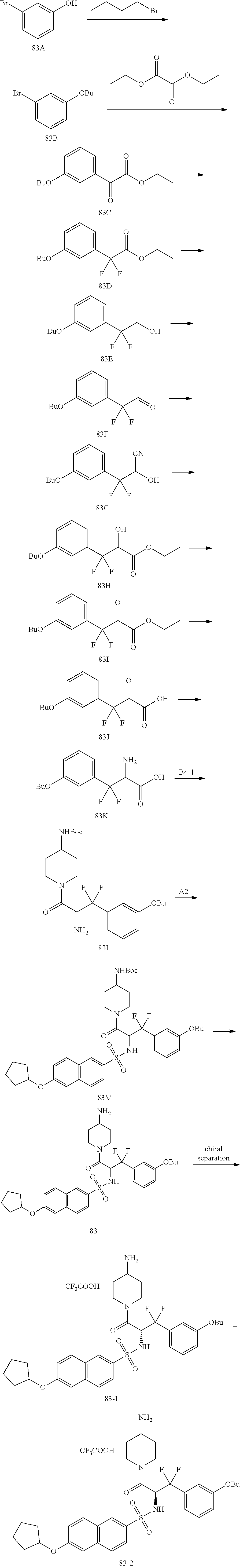

Compounds 83B, 83C, 83D, 83E, 83F, 83G, 83H, 83I, 83J, 83K, 83L, 83M, and 83 were synthesized by employing the procedures described for Compounds 4D, 9B, Compound B2-3, Compound 51E, Compound B6-3, Compounds 45F, 45G, Compound B6-3, Compounds 451, 80A, Compound B4, Compounds 2D, and 1 using Compounds 83A using K₂CO₃ as base, 83B, 83C, 83D using EtOH as solvent, 83E, 83F, 83G, 83H, 83I, 83J, 83K, 83L, Compound A2, and Compound 83M in lieu of Compounds 4C using Cs₂CO₃ as base, 9A, Compound B2-2, Compound 51D using MeOH as solvent, Compound B6-2, Compounds 45E, 45F, Compound B6-2, Compounds 45H, 51J, Compounds B2-6, B2, Compounds 2C, and 1.

Compound 83B was used for the next step without further purification. ¹H-NMR (DMSO-d₆, 400 MHz): δ (ppm) 0.98 (t, J=7.2 Hz, 3H), 1.42-1.51 (m, 2H), 1.72-1.79 (m, 2H), 3.93 (t, J=6.4 Hz, 2H), 6.81-6.83 (m, 1H), 7.05-7.15 (m, 3H).

Compound 83C: LC-MS (ESI) m/z: 251 [M+H]⁺; ¹H-NMR (CDCl₃, 400 MHz): δ (ppm) 0.96-1.01 (m, 3H), 1.42-1.45 (m, 3H), 1.47-1.52 (m, 2H), 1.74-1.81 (m, 2H), 3.99-4.02 (m, 2H), 4.41-4.46 (m, 2H), 7.18-7.20 (m, 1H), 7.38-7.42 (m, 1H), 7.51-7.55 (m, 2H).

Compound 83D: LC-MS (ESI) m/z: 273 [M+H]⁺.

Compound 83E: LC-MS (ESI) m/z: 231 [M+H]⁺; ¹H-NMR (CDCl₃, 400 MHz): δ (ppm) 0.90 (t, J=7.2 Hz, 3H), 1.49-1.51 (m, 2H), 1.76-1.79 (m, 2H), 3.92-3.99 (m, 4H), 6.97-7.07 (m, 3H), 7.34 (t, J=8.0 Hz, 1H).

Compound 83F was used for the next step without further purification. LC-MS (ESI) m/z: 229 [M+H]⁺.

Compound 83G: LC-MS (ESI) m/z: 256 [M+H]⁺.

Compound 83H: LC-MS (ESI) m/z: 325 [M+Na]⁺; ¹H-NMR (CDCl₃, 400 MHz): δ (ppm) 0.98 (t, J=7.2 Hz, 3H), 1.27 (t, J=7.2 Hz, 3H), 1.48-1.50 (m, 2H), 1.75-1.79 (m, 2H), 3.27 (d, J=8.0 Hz, 1H), 3.97 (t, J=6.8 Hz, 2H), 4.25-4.27 (m, 2H), 4.49-4.56 (m, 1H), 6.97-7.05 (m, 3H), 7.26-7.34 (m, 1H).

Compound 831: LC-MS (ESI) m/z: 301 [M+H]⁺; ¹H-NMR (CDCl₃, 400 MHz): δ (ppm) 0.97 (t, J=7.2 Hz, 3H), 1.39 (q, J=6.8 Hz, 3H), 1.48-1.49 (m, 2H), 1.76-1.78 (m, 2H), 3.97-3.99 (m, 2H), 4.38-4.39 (m, 2H), 6.97-7.00 (m, 1H), 7.12-7.14 (m, 2H), 7.38-7.39 (m, 1H).

Compound 83J was used for the next step without further purification. LC-MS (ESI) m/z: 273 [M+H]⁺.

Compound 83K: LC-MS (ESI) m/z: 274 [M+H]⁺; ¹H-NMR (CD₃OD, 400 MHz): δ (ppm) 0.98-1.02 (m, 3H), 1.50-1.56 (m, 2H), 1.77-1.80 (m, 2H), 4.02-4.05 (m, 2H), 4.88-4.89 (m, 1H), 7.11-7.16 (m, 3H), 7.41-7.45 (m, 1H).

Compound 83L: LC-MS (ESI) m/z: 456 [M+H]⁺; ¹H-NMR (CDCl₃, 400 MHz): δ (ppm) 0.96-1.00 (m, 3H), 1.42-1.92 (m, 17H), 2.70-3.06 (m, 2H), 3.61-3.76 (m, 2H), 3.94-3.99 (m, 2H), 4.29-4.54 (m, 2H), 6.94-7.02 (m, 3H), 7.31-7.33 (m, 1H).

Compound 83M: LC-MS (ESI) m/z: 674 [M−55]⁺.

Compound 83 was separated with chiral HPLC to yield Compound 83-1 and Compound 83-2.

Compound 83-1: LC-MS (ESI) m/z: 630 [M+H]⁺; ¹H-NMR (CD₃OD, 400 MHz): δ (ppm) 0.97-1.00 (m, 3H), 1.38-2.06 (m, 16H), 2.75-3.14 (m, 3H), 3.38-3.83 (m, 2H), 4.13-4.58 (m, 2H), 4.97-5.04 (m, 2H), 6.67-6.72 (m, 1H), 6.88-7.04 (m, 2H), 7.11-7.23 (m, 3H), 7.45-7.56 (m, 1H), 7.68-7.85 (m, 2H), 8.03-8.13 (m, 1H). Chiral separation condition: MeOH contained 0.5% NH₄OH; AD-H (4.6×250 mm 5 μm); retention time: 3.91 minute.

Compound 83-2: LC-MS (ESI) m/z: 630 [M+H]⁺; ¹H-NMR (CD₃OD, 400 MHz): δ (ppm) 0.96-1.00 (m, 3H), 1.38-2.06 (m, 16H), 2.75-3.14 (m, 3H), 3.38-3.83 (m, 2H), 4.13-4.58 (m, 2H), 4.97-5.04 (m, 2H), 6.67-6.72 (m, 1H), 6.88-7.04 (m, 2H), 7.11-7.23 (m, 3H), 7.45-7.56 (m, 1H), 7.68-7.85 (m, 2H), 8.03-8.13 (m, 1H). Chiral separation condition: MeOH contained 0.5% NH₄OH; AD-H (4.6×250 mm 5 μm); retention time: 2.91 minute.

### Example 84

### Synthesis of N-((2R)-3-(3-amino-8-azabicyclo[3.2.1]octan-8-yl)-1,1-difluoro-3-oxo-1-(4-phenoxyphenyl)propan-2-yl)-4-(cyclohexylmethoxy)benzenesulfonamide (Compound 84-1), and N-((2S)-3-(3-amino-8-azabicyclo[3.2.1]octan-8-yl)-1,1-difluoro-3-oxo-1-(4-phenoxyphenyl)propan-2-yl)-4-(cyclohexylmethoxy) benzenesulfonamide (Compound 84-2)

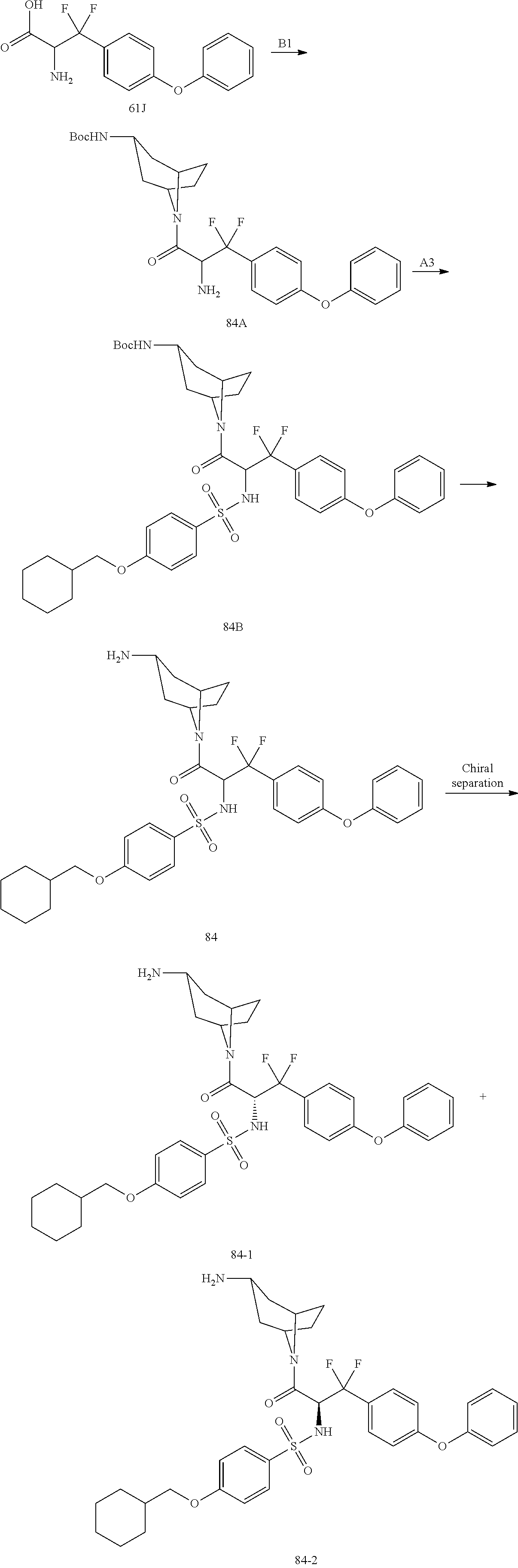

Compounds 84A, 84B, and 84 were synthesized by employing the procedures described for Compound B4, Compounds 2D, and 1 using Compound 61J, Compounds B1, A3, Compounds 84A, and 84B in lieu of Compounds B2-6, B4-1, Compound 2C, Compound B2, and Compound 1A.

Compound 84A: LC-MS (ESI) m/z: 502 [M+H]⁺.

Compound 84B: LC-MS (ESI) m/z: 698 [M−55]⁺.

Compound 84 was separated with chiral HPLC to furnish Compound 84-1 and Compound 84-2.

Compound 84-1: LC-MS (ESI) m/z: 654 [M+H]⁺; ¹H-NMR (CD₃OD, 400 MHz): δ (ppm) 0.85-1.41 (m, 8H), 1.48-1.88 (m, 12H), 2.98-3.12 (m, 1H), 3.80-3.86 (m, 2H), 4.17-4.85 (m, 3H), 6.89-7.01 (m, 6H), 7.18-7.21 (m, 1H), 7.36-7.49 (m, 4H), 7.65-7.73 (m, 2H). Chiral separation condition: MeOH contained 0.5% NH₄OH; IC (250×4.6 mm, 5 μm); retention time: 5.78 minute.

Compound 84-2: LC-MS (ESI) m/z: 654 [M+H]⁺; ¹H-NMR (CD₃OD, 400 MHz): δ (ppm) 0.85-1.41 (m, 8H), 1.48-1.88 (m, 12H), 2.98-3.12 (m, 1H), 3.80-3.86 (m, 2H), 4.17-4.85 (m, 3H), 6.89-7.01 (m, 6H), 7.18-7.21 (m, 1H), 7.36-7.49 (m, 4H), 7.65-7.73 (m, 2H). Chiral separation condition: MeOH contained 0.5% NH₄OH; IC (250×4.6 mm, 5 μm); retention time: 4.63 minute.

### Example 85

### Synthesis of N-((2R)-3-(3-amino-8-azabicyclo[3.2.1]octan-8-yl)-1,1-difluoro-3-oxo-1-(4-phenoxyphenyl)propan-2-yl)-4-(cyclohexylmethoxy)-N-methylbenzene sulfonamide trifluoroacetate (Compound 85-1), and N-((2S)-3-(3-amino-8-azabicyclo[3.2.1]octan-8-yl)-1,1-difluoro-3-oxo-1-(4-phenoxyphenyl)propan-2-yl)-4-(cyclohexylmethoxy)-N-methylbenzenesulfonamide trifluoroacetate (Compound 85-2)

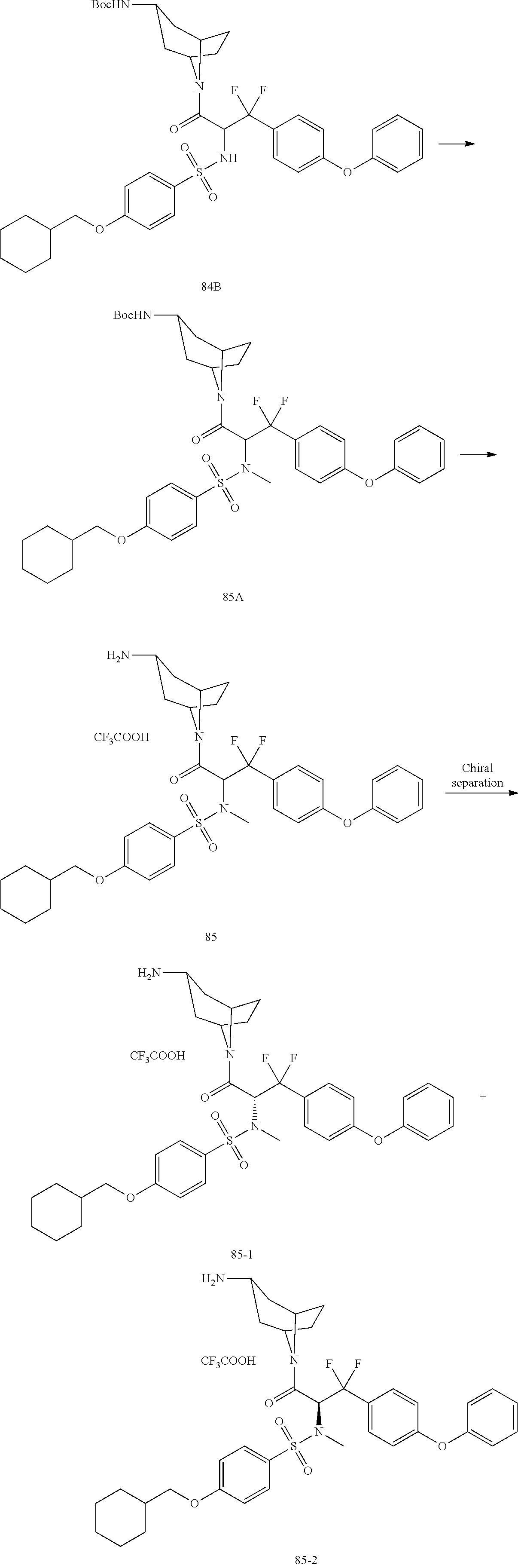

Compounds 85A and 85 were synthesized by employing the procedures described for Compounds 34A and 1 using Compounds 84B and 85A in lieu of Compounds 5A and 1A.

Compound 85A was directly used for the next step without further purification. LC-MS (ESI) m/z: 712 [M−55]⁺.

Compound 85 was separated with chiral HPLC to furnish Compound 85-1 and Compound 85-2.

Compound 85-1: LC-MS (ESI) m/z: 668 [M+H]⁺; ¹H-NMR (CD₃OD, 400 MHz): δ (ppm) 0.86-1.41 (m, 6H), 1.65-2.24 (m, 13H), 3.06 (s, 3H), 3.61-3.75 (m, 1H), 3.85-3.89 (m, 2H), 4.46-4.86 (m, 2H), 5.47-5.54 (m, 1H), 6.97-7.09 (m, 6H), 7.20-7.24 (m, 1H), 7.41-7.71 (m, 6H). Chiral separation condition: n-hexane/EtOH contained 0.1% DEA, 40/60; OZ-H (250×4.6 mm 5 μm); retention time: 8.64 minute.

Compound 85-2: LC-MS (ESI) m/z: 668 [M+H]⁺; ¹H-NMR (CD₃OD, 400 MHz): δ (ppm) 0.88-1.41 (m, 6H), 1.65-2.25 (m, 13H), 3.06 (s, 3H), 3.61-3.75 (m, 1H), 3.83-3.89 (m, 2H), 4.46-4.95 (m, 2H), 5.47-5.54 (m, 1H), 6.97-7.09 (m, 6H), 7.20-7.24 (m, 1H), 7.41-7.71 (m, 6H). Chiral separation condition: n-hexane/EtOH contained 0.1% DEA, 40/60; OZ-H (250×4.6 mm 5 μm); retention time: 11.12 minute.

### Example 86

### Synthesis of N-((2R)-3-(3-amino-8-azabicyclo[3.2.1]octan-8-yl)-1,1-difluoro-3-oxo-1-(4-phenoxyphenyl)propan-2-yl)-6-(cyclopentyloxy)naphthalene-2-sulfonamide trifluoroacetate (Compound 86-1), and N-((2S)-3-(3-amino-8-azabicyclo[3.2.1]octan-8-yl)-1,1-difluoro-3-oxo-1-(4-phenoxyphenyl)propan-2-yl)-6-(cyclopentyloxy)naphthalene-2-sulfonamide trifluoroacetate (Compound 86-2)

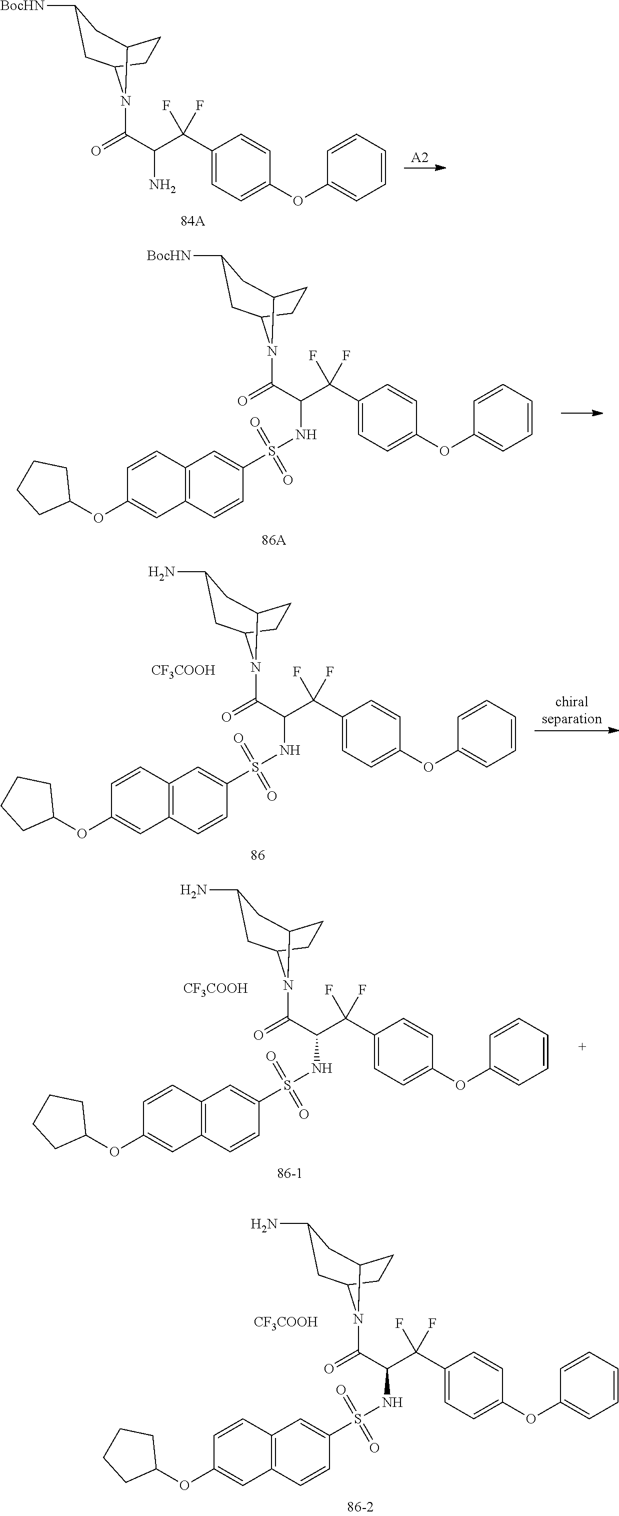

Compounds 86A and 86 were synthesized by employing the procedures described for Compounds 2D and 1 using Compound 84A, Compound A2, and Compound 86A in lieu of Compound B2, Compounds 2C, and 1A.

Compound 86A: LC-MS (ESI) m/z: 720 [M−55]⁺.

Compound 86 was separated with chiral HPLC to furnish Compound 86-1 and Compound 86-2.

Compound 86-1: LC-MS (ESI) m/z: 676 [M+H]⁺; ¹H-NMR (CD₃OD, 400 MHz): δ (ppm) 0.68-0.92 (m, 1H), 1.28-1.38 (m, 2H), 1.52-2.15 (m, 13H), 3.49-3.73 (m, 1H), 4.32-4.71 (m, 3H), 4.96-4.98 (m, 1H), 6.66-7.03 (m, 4H), 7.15-7.28 (m, 4H), 7.34-7.42 (m, 2H), 7.48-7.65 (m, 2H), 7.77-8.21 (m, 3H). Chiral separation condition: MeOH contained 0.5% NH₄OH; IC (250×4.6 mm, 5 μm); retention time: 5.46 minute.

Compound 86-2: LC-MS (ESI) m/z: 676 [M+H]⁺; ¹H-NMR (CD₃OD, 400 MHz): δ (ppm) 0.68-0.92 (m, 1H), 1.28-1.38 (m, 2H), 1.52-2.15 (m, 13H), 3.49-3.73 (m, 1H), 4.32-4.71 (m, 3H), 4.96-4.98 (m, 1H), 6.66-7.03 (m, 4H), 7.15-7.28 (m, 4H), 7.34-7.42 (m, 2H), 7.48-7.65 (m, 2H), 7.77-8.21 (m, 3H). Chiral separation condition: MeOH contained 0.5% NH₄OH; IC (250×4.6 mm, 5 μm); retention time: 4.03 minute.

### Example 87

### Synthesis of N-((2R)-3-(3-amino-8-azabicyclo[3.2.1]octan-8-yl)-1,1-difluoro-3-oxo-1-(4-phenoxyphenyl)propan-2-yl)-6-(cyclopentyloxy)-N-methylnaphthalene-2-sulfonamide trifluoroacetate (Compound 87-1), and N-((2S)-3-(3-amino-8-azabicyclo[3.2.1]octan-8-yl)-1,1-difluoro-3-oxo-1-(4-phenoxyphenyl)propan-2-yl)-6-(cyclopentyloxy)-N-methylnaphthalene-2-sulfonamide trifluoroacetate (Compound 87-2)

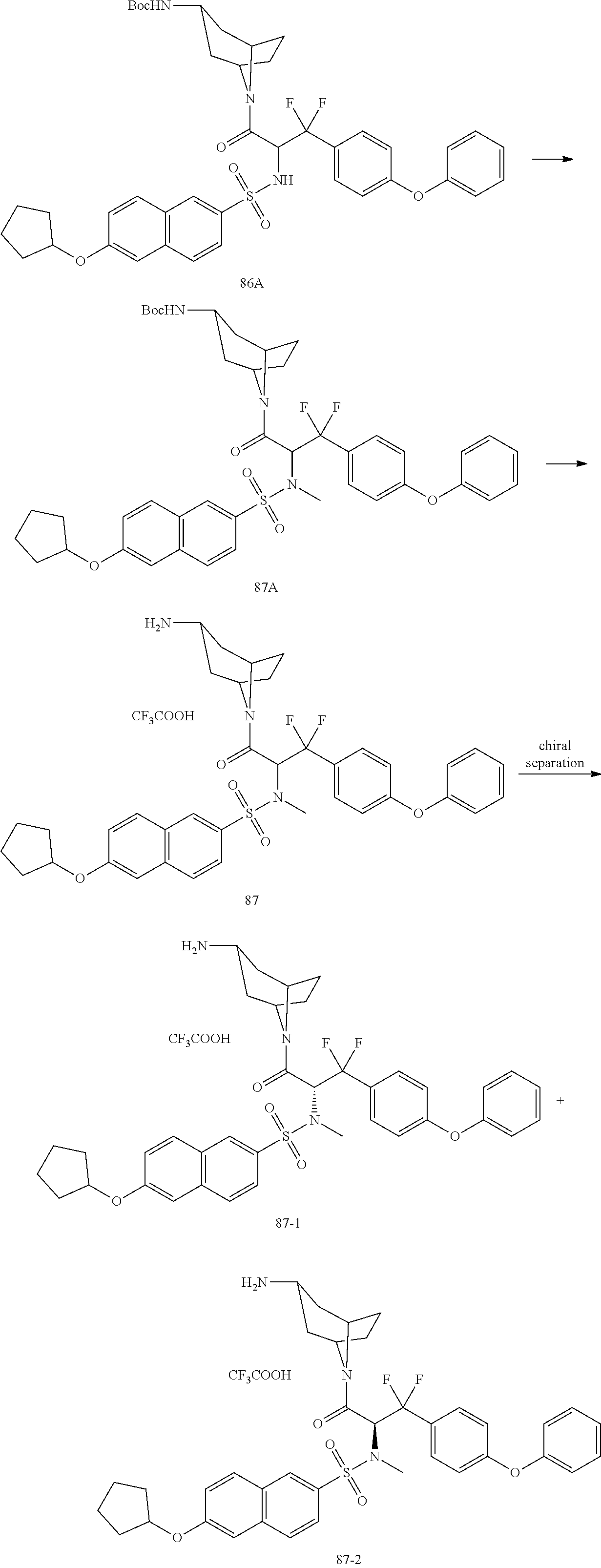

Compounds 87A and 87 were synthesized by employing the procedures described for Compounds 34A and 1 using Compounds 86A and 87A in lieu of Compounds 5A and 1A.

Compound 87A was directly used for the next step without further purification. LC-MS (ESI) m/z: 734 [M−55]⁺.

Compound 87 was separated with chiral HPLC to furnish Compound 87-1 and Compound 87-2.

Compound 87-1: LC-MS (ESI) m/z: 690 [M+H]⁺; ¹H-NMR (CD₃OD, 400 MHz): δ (ppm) 1.27-2.21 (m, 16H), 3.08 (s, 3H), 3.63-3.72 (m, 1H), 4.54-4.72 (m, 2H), 4.91-5.03 (m, 1H), 5.54-5.62 (m, 1H), 6.88-7.02 (m, 4H), 7.19-7.27 (m, 3H), 7.32-7.66 (m, 5H), 7.81-8.29 (m, 3H). Chiral separation condition: n-hexane/EtOH contained 0.1% DEA, 30/70; AY-H (250×4.6 mm 5 μm); retention time: 22.85 minute.

Compound 87-2: LC-MS (ESI) m/z: 690 [M+H]⁺; ¹H-NMR (CD₃OD, 400 MHz): δ (ppm) 1.29-2.20 (m, 16H), 3.08 (s, 3H), 3.63-3.72 (m, 1H), 4.53-4.72 (m, 2H), 4.91-5.03 (m, 1H), 5.54-5.62 (m, 1H), 6.88-7.02 (m, 4H), 7.19-7.27 (m, 3H), 7.32-7.66 (m, 5H), 7.81-8.29 (m, 3H). Chiral separation condition: n-hexane/EtOH contained 0.1% DEA, 30/70; AY-H (250×4.6 mm 5 μm); retention time: 7.42 minute.

### Example 88

### Synthesis of N-((2R)-3-(3-amino-8-azabicyclo[3.2.1]octan-8-yl)-1-(4-chlorophenyl)-1,1-difluoro-3-oxopropan-2-yl)-1-(cyclopentylmethyl)-1H-indazole-5-sulfonamide (Compound 88-1), and N-((2S)-3-(3-amino-8-azabicyclo[3.2.1]octan-8-yl)-1-(4-chlorophenyl)-1,1-difluoro-3-oxopropan-2-yl)-1-(cyclopentylmethyl)-1H-indazole-5-sulfonamide (Compound 88-2)

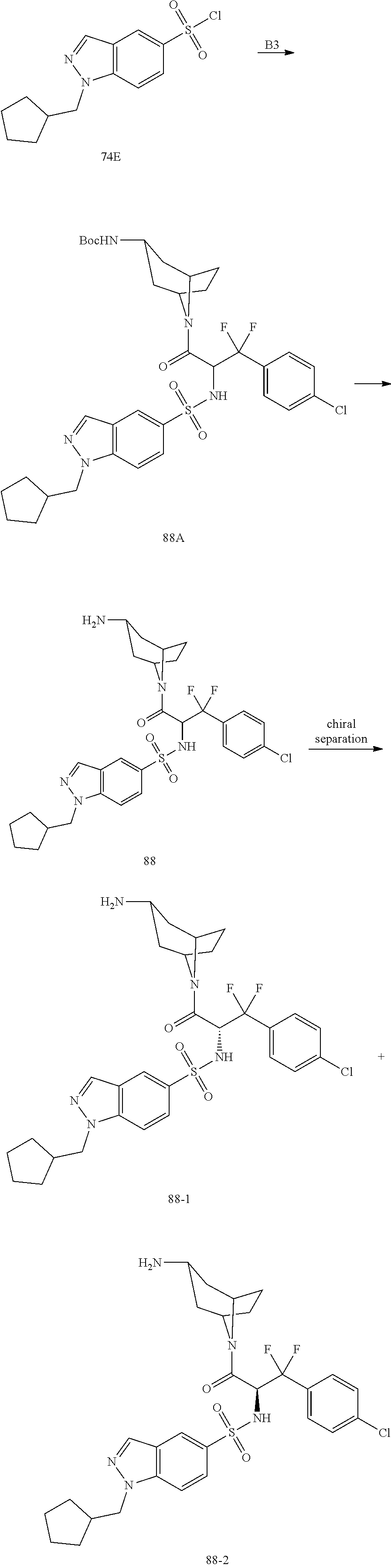

Compounds 88A and 88 were synthesized by employing the procedures described for Compounds 2D and 1 using Compound B3, Compounds 74E, and 88A in lieu of Compound B2, Compounds 2C, and 1A.

Compound 88A: LC-MS (ESI) m/z: 650 [M−55]⁺.

Compound 88 was separated with chiral HPLC to give Compound 88-1 and Compound 88-2.

Compound 88-1: LC-MS (ESI) m/z: 606 [M+H]⁺; ¹H-NMR (CD₃OD, 400 MHz): δ (ppm) 0.58-1.18 (m, 2H), 1.18-1.31 (m, 3H), 1.40-1.78 (m, 11H), 2.41-2.48 (m, 1H), 2.86-2.97 (m, 1H), 4.14-4.35 (m, 4H), 4.53-4.67 (m, 1H), 7.18-7.31 (m, 4H), 7.56-7.61 (m, 2H), 8.06-8.11 (m, 2H). Chiral separation condition: MeOH contained 0.5% NH₄OH; IC (4.6×250 mm 5 μm); retention time: 4.68 minute.

Compound 88-2: LC-MS (ESI) m/z: 606 [M+H]⁺; ¹H-NMR (CD₃OD, 400 MHz): δ (ppm) 0.70-1.18 (m, 2H), 1.23-1.30 (m, 3H), 1.45-1.81 (m, 11H), 2.42-2.48 (m, 1H), 2.85-2.97 (m, 1H), 4.21-4.34 (m, 4H), 4.45-4.67 (m, 1H), 7.13-7.41 (m, 4H), 7.47-7.63 (m, 2H), 8.01-8.18 (m, 2H). Chiral separation condition: MeOH contained 0.5% NH₄OH; IC (4.6×250 mm 5 μm); retention time: 3.81 minute.

### Example 89

### Synthesis of N-((2R)-3-(3-amino-8-azabicyclo[3.2.1]octan-8-yl)-1-(4-bromo-3-methoxyphenyl)-1,1-difluoro-3-oxopropan-2-yl)-6-(cyclopentyloxy)naphthalene-2-sulfonamide (Compound 89-1), and N-((2S)-3-(3-amino-8-azabicyclo[3.2.1]octan-8-yl)-1-(4-bromo-3-methoxyphenyl)-1,1-difluoro-3-oxopropan-2-yl)-6-(cyclopentyloxy)naphthalene-2-sulfonamide (Compound 89-2)

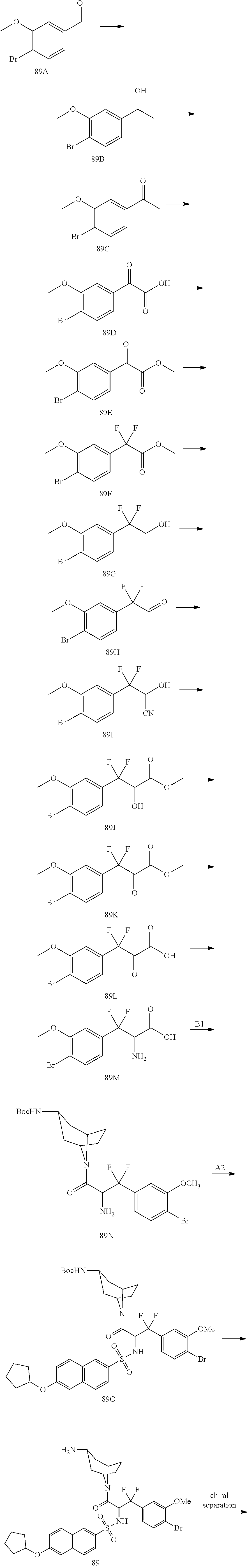

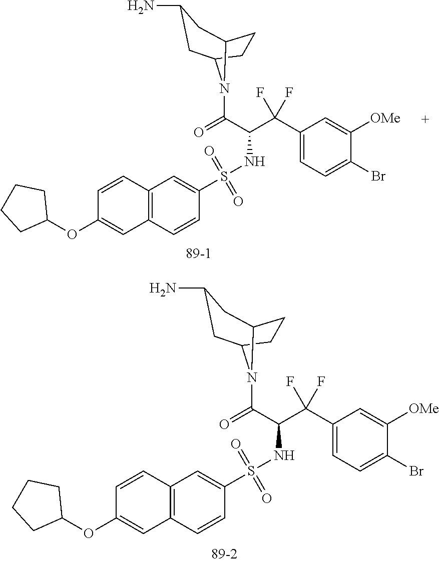 [SMILES: COc1cc(C(F)(F)[C@H](NS(=O)(=O)c2ccc3cc(OC4CCCC4)ccc3c2)C(=O)N2C3CCC2CC(N)C3)ccc1Br ; COc1cc(C(F)(F)[C@@H](NS(=O)(=O)c2ccc3cc(OC4CCCC4)ccc3c2)C(=O)N2C3CCC2CC(N)C3)ccc1Br]

To a stirred solution of Compound 4-bromo-3-methoxybenzaldehyde 89A (20 g, 93 mmol) in THF (200 mL) was dropped methylmagnesium bromide solution (3 Min ether, 62 mL, 187 mmol) at −30° C. The reaction mixture was stirred at 25° C. for 4 hours, quenched with ice water (100 mL), filtered, and extracted with dichloromethane (100 mL×3). The combined organic extracts was dried over anhydrous sodium sulfate, filtered, and concentrated. The residue was purified by silica gel column chromatography to give Compound 89B. ¹H-NMR (CDCl₃, 400 MHz): δ (ppm) 1.32 (d, J=6.4 Hz, 3H), 3.84 (s, 3H), 4.70-4.76 (m, 1H), 5.26 (d, J=4.4 Hz, 1H), 6.84-6.89 (m, 1H), 7.08 (d, J=1.6 Hz, 1H) 7.49 (d, J=8.0 Hz, 1H).

Compounds 89C and 89D were synthesized by employing the procedures described for Compound B6-3 and Compound 42B using Compounds 89B and 89C in lieu of for Compound B6-2 and Compound 42A. Compound 89C: LC-MS (ESI) m/z: No; ¹H-NMR (CDCl₃, 400 MHz): δ (ppm) 2.57 (s, 3H), 3.94 (d, J=6.0 Hz, 3H), 7.36-7.38 (m, 1H), 7.46 (d, J=1.6 Hz, 1H), 7.61 (d, J=8.0 Hz, 1H).

Compound 89D: LC-MS (ESI) m/z: 259 [M+H]⁺.

To a solution of Compound 89D (10.5 g, 40 mmol) in methanol (100 mL) was dropped SOCl₂ (6 mL, 80 mmol) at 0° C. The reaction mixture was stirred at room temperature for 2 hours. After removal of the solvent, the residue was diluted with dichloromethane (50 mL) and washed with saturated aqueous sodium bicarbonate solution (50 mL). The organic layer was separated, dried over anhydrous sodium sulfate, filtered, and concentrated. The residue was purified by silica gel column chromatography to give Compound 89E. ¹H-NMR (CDCl₃, 400 MHz): δ (ppm) 3.97 (s, 3H), 3.99 (s, 3H), 7.50 (d, J=2.0 Hz, 1H), 7.57 (d, J=1.6 Hz, 1H), 7.70 (d, J=8.0 Hz, 1H).

Compound 89F was synthesized by employing the procedure described for Compound B2-3 using Compound 89E in lieu of for Compound B2-2. ¹H-NMR (CDCl₃, 400 MHz): δ (ppm) 3.86 (s, 3H), 3.94 (s, 3H), 7.10-7.06 (m, 2H), 7.63 (d, J=8.4 Hz, 1H).

To a solution of Compound 89F (7.5 g, 25.4 mmol) in ethanol (100 mL) at 0° C. was added NaBH₄ (2.9 g, 76 mmol) and CaCl₂ (8.4 g, 76 mmol). The reaction mixture was stirred at 40° C. for 2 hours, quenched with diluted HCl solution (1 M, 50 mL) and extracted with dichloromethane (100 mL×2). The combined organic extracts was washed with brine (100 mL), dried over anhydrous sodium sulfate, filtered, and concentrated. The residue was purified by silica gel column chromatography to give Compound 89G. LC-MS (ESI) m/z: 249 [M−OH]⁺; ¹H-NMR (CDCl₃, 400 MHz): δ (ppm) 4.00-4.23 (m, 5H), 7.00 (d, J=8.0 Hz, 1H), 7.04 (d, J=1.6 Hz, 1H), 7.52 (d, J=8.4 Hz, 1H).

Compounds 89H, 89I, 89J, 89K, 89L, 89M, 89N, 890, and 89 were synthesized by employing the procedures described for Compound B6-3, Compounds 45F, 45G, Compound B6-3, Compounds 451, 80A, Compound B4, Compounds 1A, and 1 using Compounds 89G, 89H, 89I in MeOH, 89J, 89K, 89L, 89M, Compound B1, Compound 89N, Compound A2, and Compound 890 in lieu of for Compound B6-2, Compounds 45E, 45F in EtOH, Compound B6-2, Compounds 4511, 51J, Compounds B2-6, B4-1, A1, B2, and Compound 1A.

Compound 89H was used for the next step without further purification. LC-MS (ESI) m/z: 265 [M+H]⁺.

Compound 891: ¹H-NMR (CDCl₃, 400 MHz): δ (ppm) 3.93 (s, 3H), 4.27 (s, 1H), 4.79-4.83 (t, J=8.8 Hz, 1H), 7.02-7.04 (m, 1H), 7.06-7.07 (m, 1H), 7.63 (d, J=8.0 Hz, 1H).

Compound 89J: LC-MS (ESI) m/z: 347 [M+Na]⁺; ¹H-NMR (CDCl₃, 400 MHz): δ (ppm) 3.29 (d, J=7.6 Hz, 1H), 3.85 (s, 3H), 3.92 (s, 3H), 4.50-4.57 (m, 1H), 6.94-7.00 (m, 2H), 7.59 (d, J=8.4 Hz, 1H).

Compound 89K: LC-MS (ESI) m/z: non-ionizable compound under routine conditions used; ¹H-NMR (CDCl₃, 400 MHz): δ (ppm) 3.92 (s, 3H), 3.96 (s, 3H), 7.05-7.07 (m, 1H), 7.11 (d, J=1.6 Hz, 1H), 7.59 (d, J=8.4 Hz, 1H).

Compound 89L: LC-MS (ESI) m/z: non-ionizable compound under routine conditions used; ¹H-NMR (CD₃OD, 400 MHz): δ (ppm) 3.88 (s, 3H), 7.02-7.04 (m, 1H), 7.15 (s, 1H), 7.55 (d, J=8.0 Hz, 1H).

Compound 89M: LC-MS (ESI) m/z: non-ionizable compound under routine conditions used; ¹H-NMR (DMSO-d₆, 400 MHz): δ (ppm) 3.91 (s, 3H), 5.04 (m, 1H), 7.07 (m, 1H), 7.30 (s, 1H), 7.74 (d, J=8.0 Hz, 1H).

Compound 89N: LC-MS (ESI) m/z: 518 [M+H]⁺.

Compound 890: LC-MS (ESI) m/z: 814 [M+Na]⁺.

Compound 89 was separated with chiral HPLC to give Compound 89-1 and Compound 89-2.

Compound 89-1: LC-MS (ESI) m/z: 692 [M+H]⁺; ¹H-NMR (CD₃OD, 400 MHz): δ (ppm) 0.60-1.29 (m, 4H), 1.70-2.05 (m, 12H), 2.83-3.05 (m, 1H), 3.79-3.81 (m, 3H), 4.23-4.42 (m, 2H), 4.63-4.74 (m, 1H), 4.97-5.01 (m, 1H), 6.93-7.06 (m, 2H), 7.20-7.31 (m, 2H), 7.48-7.65 (m, 2H), 7.75-7.90 (m, 2H), 8.19-8.25 (m, 1H). Chiral separation condition: MeOH contained 0.1% NH₄OH; IC (4.6×150 mm 5 μm); retention time: 4.07 minute.

Compound 89-2: LC-MS (ESI) m/z: 692 [M+H]⁺; ¹H-NMR (CD₃OD, 400 MHz): δ (ppm) 0.57-1.37 (m, 4H), 1.48-2.04 (m, 12H), 2.82-3.03 (m, 1H), 3.79-3.80 (m, 3H), 4.22-4.41 (m, 2H), 4.63-4.74 (m, 1H), 4.97-5.01 (m, 1H), 6.93-7.06 (m, 2H), 7.20-7.30 (m, 2H), 7.48-7.66 (m, 2H), 7.74-7.89 (m, 2H), 8.19-8.25 (m, 1H). Chiral separation condition: MeOH contained 0.1% NH₄OH; IC (4.6×150 mm 5 m); retention time: 3.34 minute.

### Example 90

### Synthesis of N-((2R)-3-(3-amino-8-azabicyclo[3.2.1]octan-8-yl)-1-(3-butoxyphenyl)-1,1-difluoro-3-oxopropan-2-yl)-6-(cyclopentyloxy)naphthalene-2-sulfonamide trifluoroacetate (Compound 90-1), and N-((2S)-3-(3-amino-8-azabicyclo[3.2.1]octan-8-yl)-1-(3-butoxyphenyl)-1,1-difluoro-3-oxopropan-2-yl)-6-(cyclopentyloxy)naphthalene-2-sulfonamide trifluoroacetate (Compound 90-2)

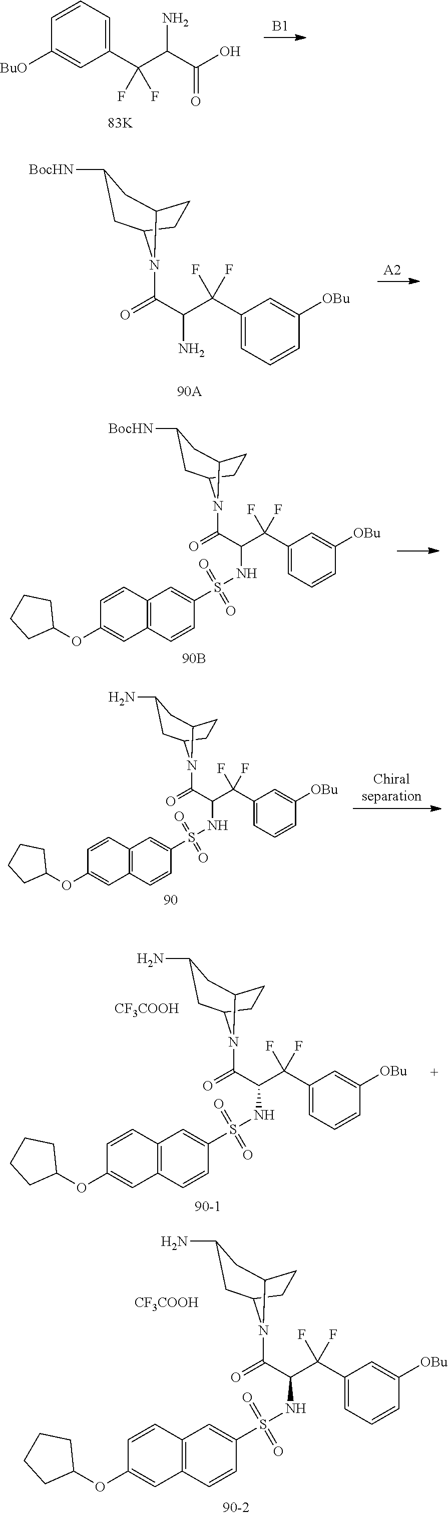

Compounds 90A, 90B, and 90 were synthesized by employing the procedures described for Compound B4, Compounds 2D, and 1 using Compound B1, Compounds 83K, 90A, Compound A2, and Compound 90B in lieu of Compounds B4-1, B2-6, B2, Compounds 2C, and 1A.

Compound 90A: LC-MS (ESI) m/z: 482 [M+H]⁺.

Compound 90B: LC-MS (ESI) m/z: 700 [M−55]⁺.

Compound 90 was separated with chiral HPLC to yield Compound 90-1 and Compound 90-2.

Compound 90-1: LC-MS (ESI) m/z: 656 [M+H]⁺; ¹H-NMR (CD₃OD, 400 MHz): δ (ppm) 0.96-1.00 (m, 3H), 1.42-2.07 (m, 20H), 3.54-3.87 (m, 3H), 4.39-4.67 (m, 3H), 5.0-5.02 (m, 1H), 6.67-6.70 (m, 1H), 6.97-7.08 (m, 2H), 7.12-7.27 (m, 3H), 7.52-7.57 (m, 1H), 7.71-7.82 (m, 2H), 8.08-8.14 (m, 1H). Chiral separation condition: n-hexane/EtOH contained 0.1% DEA, 80/20; OJ-H (250×4.6 mm 5 μm); retention time: 11.68 minute.

Compound 90-2: LC-MS (ESI) m/z: 656 [M+H]⁺; ¹H-NMR (CD₃OD, 400 MHz): δ (ppm) 0.97-1.01 (m, 3H), 1.43-2.07 (m, 20H), 3.55-3.88 (m, 3H), 4.40-4.68 (m, 3H), 5.0-5.02 (m, 1H), 6.68-6.70 (m, 1H), 6.97-7.08 (m, 2H), 7.14-7.29 (m, 3H), 7.52-7.57 (m, 1H), 7.71-7.82 (m, 2H), 8.08-8.14 (m, 1H). Chiral separation condition: n-hexane/EtOH contained 0.1% DEA, 80/20; OJ-H (250×4.6 mm 5 μm); retention time: 7.98 minute.

### Example 91

### Synthesis of (R)—N-(3-(4-aminopiperidin-1-yl)-1-(4′-chloro-[1,1′-biphenyl]-4-yl)-1,1-difluoro-3-oxopropan-2-yl)-6-(cyclopentyloxy)naphthalene-2-sulfonamide trifluoroacetate (Compound 91-1), and (S)—N-(3-(4-aminopiperidin-1-yl)-1-(4′-chloro-[1,1′-biphenyl]-4-yl)-1,1-difluoro-3-oxopropan-2-yl)-6-(cyclopentyloxy)naphthalene-2-sulfonamide trifluoroacetate (Compound 91-2)

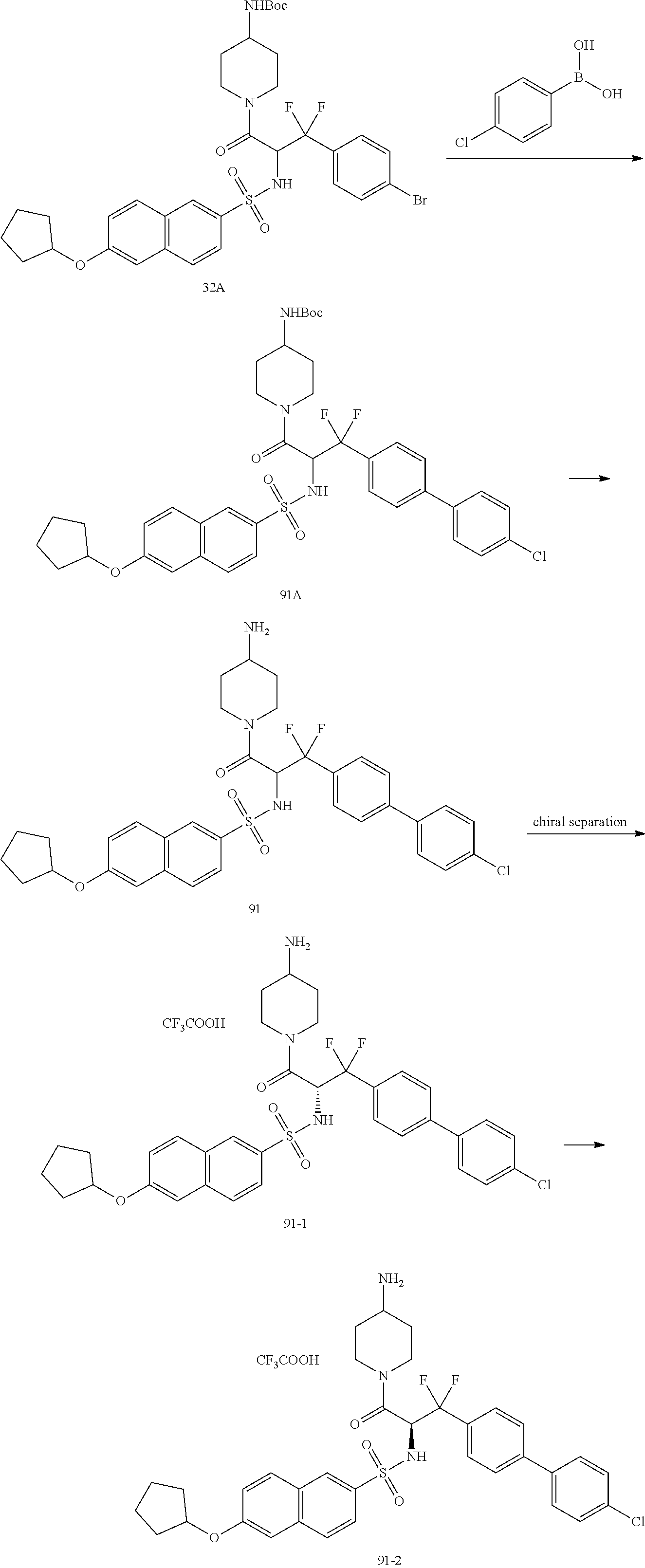

Compounds 91A and 91 were synthesized by employing the procedures described for Compounds 14C and 1 using 4-chlorophenylboronic acid, Compounds 32A, and 91A in lieu of Compounds 14B, 14A, and 1A.

Compound 91A: LC-MS (ESI) m/z: 768 [M+H]⁺.

Compound 91 was separated with chiral HPLC to furnish Compound 91-1 and Compound 91-2).

Compound 91-1: LC-MS (ESI) m/z: 668 [M+H]⁺; ¹H-NMR (CD₃OD, 400 MHz): δ (ppm) 1.37-2.32 (m, 13H), 2.80-3.13 (m, 1H), 3.26-3.49 (m, 1H), 4.24-4.80 (m, 3H), 5.00-5.12 (m, 1H), 6.89-7.54 (m, 11H), 7.67-7.82 (m, 2H), 8.00-8.10 (m, 1H). Chiral separation condition: MeOH contained 0.5% NH₄OH; R,R-Whelk-O1 (4.6×250 mm, 5 μm); retention time: 6.23 minute.

Compound 91-2: LC-MS (ESI) m/z: 668 [M+H]⁺; ¹H-NMR (CD₃OD, 400 MHz): δ (ppm) 1.28-2.27 (m, 13H), 2.80-3.12 (m, 1H), 3.26-3.48 (m, 1H), 4.21-4.78 (m, 3H), 5.01-5.12 (m, 1H), 6.87-7.52 (m, 11H), 7.64-7.80 (m, 2H), 8.01-8.11 (m, 1H). Chiral separation condition: MeOH contained 0.5% NH₄OH; R,R-Whelk-O1 (4.6×250 mm, 5 μm); retention time: 8.93 minute.

### Example 92

### Synthesis of N-((2R)-3-(3-amino-8-azabicyclo[3.2.1]octan-8-yl)-1,1-difluoro-3-oxo-1-(4-(trifluoromethyl)phenyl)propan-2-yl)-1-(cyclopentylmethyl)-1H-indazole-5-sulfonamide (Compound 92-1), and N-((2S)-3-(3-amino-8-azabicyclo[3.2.1]octan-8-yl)-1,1-difluoro-3-oxo-1-(4-(trifluoromethyl)phenyl)propan-2-yl)-1-(cyclopentylmethyl)-1H-indazole-5-sulfonamide (Compound 92-2)

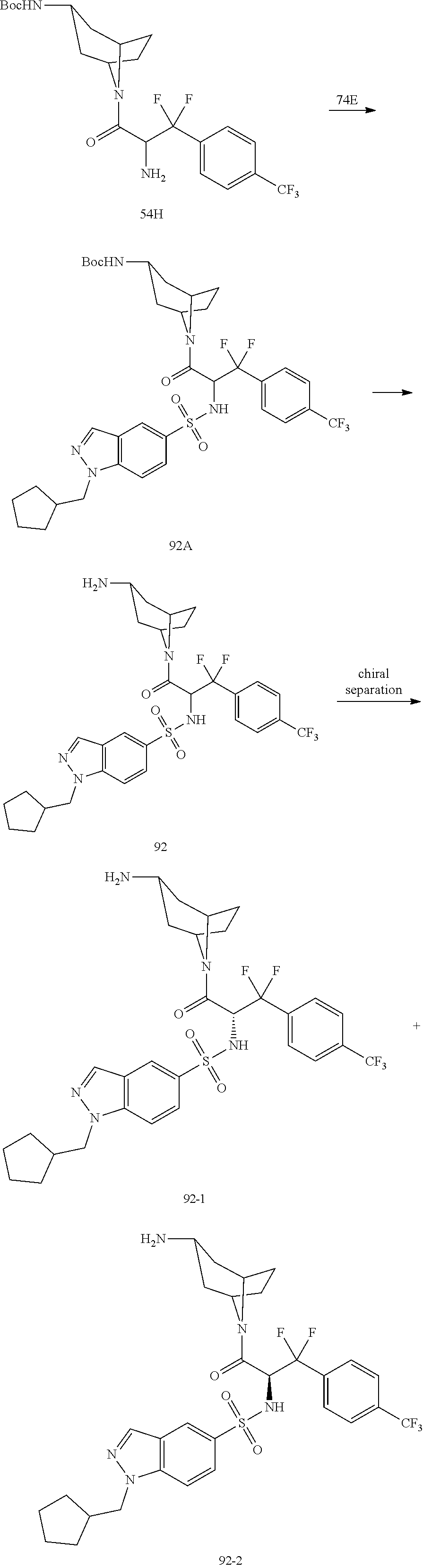 [SMILES: CC(C)(C)OC(=O)NC1CC2CCC(C1)N2C(=O)C(N)C(F)(F)c1ccc(C(F)(F)F)cc1 ; "74E" ; CC(C)(C)OC(=O)NC1CC2CCC(C1)N2C(=O)C(NS(=O)(=O)c1ccc2c(cnn2CC2CCCC2)c1)C(F)(F)c1ccc(C(F)(F)F)cc1 ; NC1CC2CCC(C1)N2C(=O)C(NS(=O)(=O)c1ccc2c(cnn2CC2CCCC2)c1)C(F)(F)c1ccc(C(F)(F)F)cc1 ; NC1CC2CCC(C1)N2C(=O)[C@@H](NS(=O)(=O)c1ccc2c(cnn2CC2CCCC2)c1)C(F)(F)c1ccc(C(F)(F)F)cc1 ; NC1CC2CCC(C1)N2C(=O)[C@H](NS(=O)(=O)c1ccc2c(cnn2CC2CCCC2)c1)C(F)(F)c1ccc(C(F)(F)F)cc1]

Compounds 92A and 92 were synthesized by employing the procedures described for Compounds 1A and 6 using Compounds 74E, 54H, and 92A in lieu of Compounds A1, B2, and Compound 6C.

Compound 92A was used for the next step without further purification. LC-MS (ESI) m/z: 640 [M−Boc+H]⁺.

Compound 92 was separated with chiral HPLC to give Compound 92-1 and Compound 92-2.

Compound 92-1: LC-MS (ESI) m/z: 640 [M+H]⁺; ¹H-NMR (CD₃OD, 400 MHz): δ (ppm) 0.70-0.99 (m, 1H), 1.22-1.39 (m, 4H), 1.55-1.88 (m, 11H), 2.50-2.58 (m, 1H), 2.94-3.08 (m, 1H), 4.23-4.42 (m, 4H), 4.70-4.85 (m, 1H), 7.65-7.75 (m, 6H), 8.17-8.28 (m, 2H); Chiral separation condition: MeOH contained 0.5% NH₄OH; IC (4.6×250 mm, 5 μm); retention time: 6.18 minute.

Compound 92-2: LC-MS (ESI) m/z: 640 [M+H]⁺; ¹H-NMR (CD₃OD, 400 MHz): δ (ppm) 0.75-1.00 (m, 1H), 1.26-1.40 (m, 4H), 1.57-1.88 (m, 11H), 2.50-2.58 (m, 1H), 2.96-3.09 (m, 1H), 4.23-4.42 (m, 4H), 4.69-4.84 (m, 1H), 7.66-7.74 (m, 6H), 8.16-8.27 (m, 2H); Chiral separation condition: MeOH contained 0.5% NH₄OH; IC (4.6×250 mm, 5 μm); retention time: 4.83 min.

### Example 93

### Synthesis of (R)—N-(3-(4-aminopiperidin-1-yl)-1,1-difluoro-3-oxo-1-(4-(trifluoromethyl)phenyl)propan-2-yl)-1-(cyclopentylmethyl)-1H-indazole-5-sulfonamide (Compound 93-1), and (S)—N-(3-(4-aminopiperidin-1-yl)-1,1-difluoro-3-oxo-1-(4-(trifluoromethyl)phenyl)propan-2-yl)-1-(cyclopentylmethyl)-1H-indazole-5-sulfonamide (Compound 93-2)

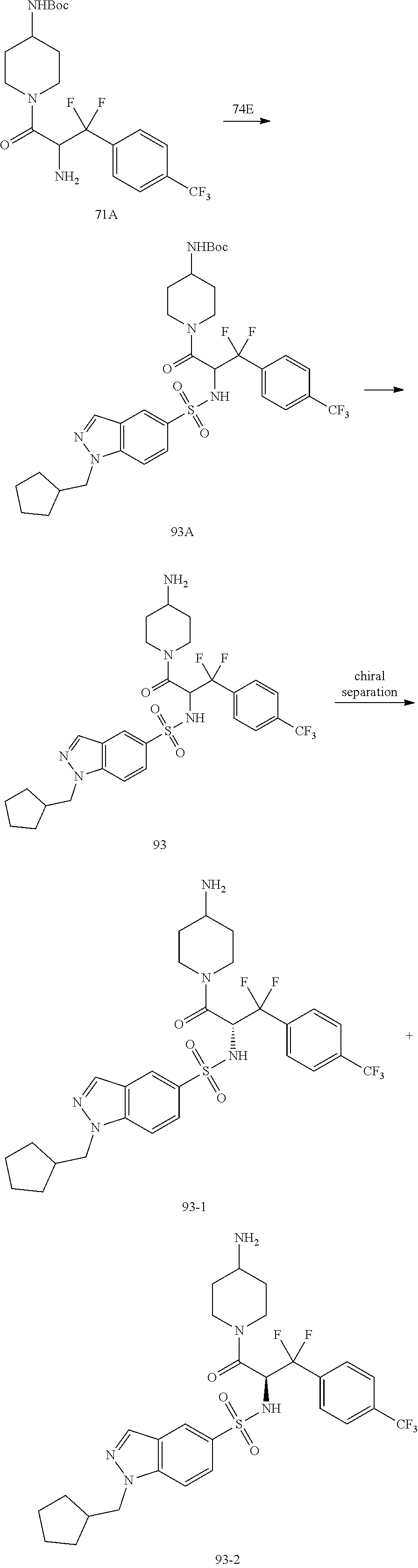

Compounds 93A and 93 were synthesized by employing the procedures described for Compounds 1A and 6 using Compounds 74E, 71A, and 93A in lieu of Compounds A1, B2, and Compound 6C.

Compound 93A was used for the next step without further purification. LC-MS (ESI) m/z: 614 [M−Boc+H]⁺.

Compound 93 was separated with chiral HPLC to give Compound 93-1 and Compound 93-1.

Compound 93-1: LC-MS (ESI) m/z: 614 [M+H]⁺; ¹H-NMR (CD₃OD, 400 MHz): δ (ppm) 0.25-1.09 (m, 2H), 1.37-1.97 (m, 11H), 2.51-2.68 (m, 2H), 2.77-2.94 (m, 1H), 3.87-4.09 (m, 2H), 4.37-4.39 (m, 2H), 4.93-5.03 (m, 1H), 7.68-7.75 (m, 6H), 8.19-8.26 (m, 2H); Chiral separation condition: MeOH contained 0.5% NH₄OH; IC (4.6×250 mm, 5 μm); retention time: 5.09 minute.

Compound 93-2: LC-MS (ESI) m/z: 614 [M+H]⁺; ¹H-NMR (CD₃OD, 400 MHz): δ (ppm) 0.28-1.09 (m, 2H), 1.31-1.97 (m, 11H), 2.48-2.68 (m, 2H), 2.77-2.95 (m, 1H), 3.87-4.09 (m, 2H), 4.37-4.39 (m, 2H), 4.93-5.03 (m, 1H), 7.68-7.75 (m, 6H), 8.19-8.26 (m, 2H). Chiral separation condition: MeOH contained 0.5% NH₄OH; IC (4.6×250 mm, 5 μm); retention time: 3.95 minute.

### Example 94

### Synthesis of N-((2R)-3-(3-amino-8-azabicyclo[3.2.1]octan-8-yl)-1-(4′-chloro-[1,1′-biphenyl]-4-yl)-1,1-difluoro-3-oxopropan-2-yl)-1-(cyclopentylmethyl)-1H-indazole-5-sulfonamide (Compound 94-1), and N-((2S)-3-(3-amino-8-azabicyclo[3.2.1]octan-8-yl)-1-(4′-chloro-[1,1′-biphenyl]-4-yl)-1,1-difluoro-3-oxopropan-2-yl)-1-(cyclopentylmethyl)-1H-indazole-5-sulfonamide (Compound 94-2)

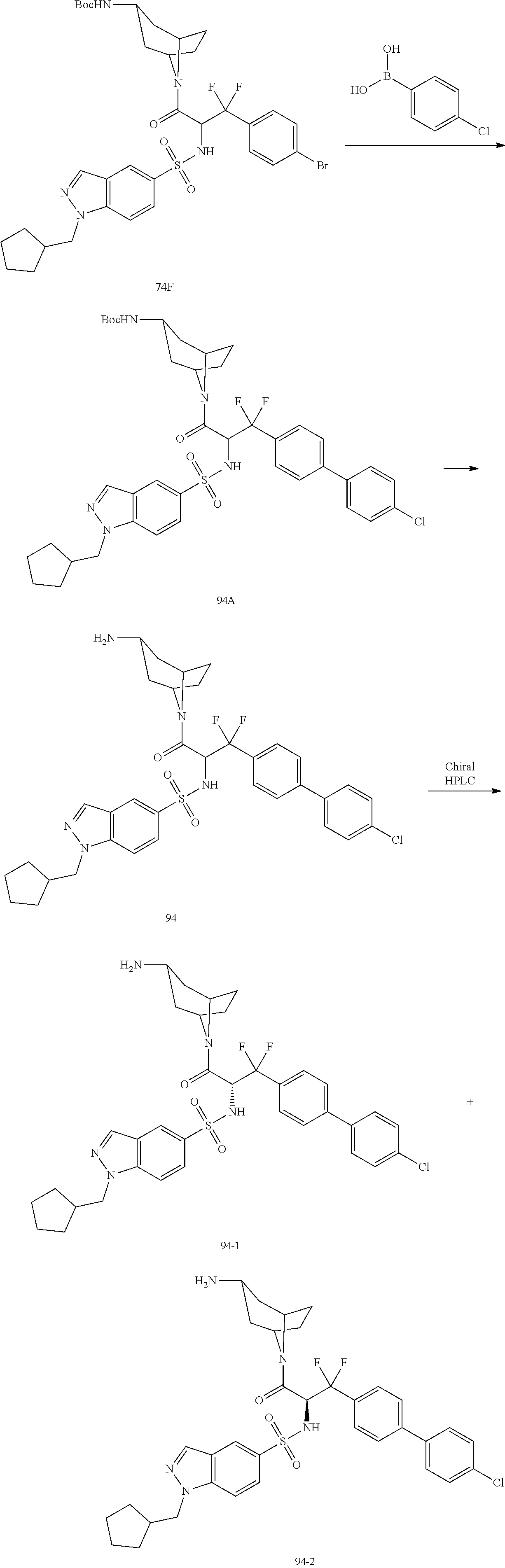 [SMILES: CC(C)(C)OC(=O)NC1CC2CCC(C1)N2C(=O)C(NS(=O)(=O)c1ccc2c(cnn2CC2CCCC2)c1)C(F)(F)c1ccc(Br)cc1 ; OB(O)c1ccc(Cl)cc1 ; CC(C)(C)OC(=O)NC1CC2CCC(C1)N2C(=O)C(NS(=O)(=O)c1ccc2c(cnn2CC2CCCC2)c1)C(F)(F)c1ccc(-c2ccc(Cl)cc2)cc1 ; NC1CC2CCC(C1)N2C(=O)C(NS(=O)(=O)c1ccc2c(cnn2CC2CCCC2)c1)C(F)(F)c1ccc(-c2ccc(Cl)cc2)cc1 ; NC1CC2CCC(C1)N2C(=O)[C@@H](NS(=O)(=O)c1ccc2c(cnn2CC2CCCC2)c1)C(F)(F)c1ccc(-c2ccc(Cl)cc2)cc1 ; NC1CC2CCC(C1)N2C(=O)[C@H](NS(=O)(=O)c1ccc2c(cnn2CC2CCCC2)c1)C(F)(F)c1ccc(-c2ccc(Cl)cc2)cc1]

Compounds 94A and 94 were synthesized by employing the procedures described for Compounds 14 and 1 using 4-chlorophenylboronic acid, Compounds 74F, and 94A in lieu of Compounds 14B, 14A, and 1A.

Compound 94A: LC-MS (ESI) m/z: 694 [M−55]⁺.

Compound 94 was separated with chiral HPLC (Chiral separation condition: n-hexane/EtOH contained 0.1% DEA, 30/70; IC, 250×4.6 mm, 5 μm) to give Compound 94-1 and Compound 94-2.

Compound 94-1: LC-MS (ESI) m/z: 682 [M+H]⁺; ¹H-NMR (CD₃OD, 400 MHz): δ (ppm) 0.81-1.28 (m, 4H), 1.38-1.93 (m, 12H), 2.41-2.48 (m, 1H), 3.08-3.17 (m, 1H), 4.07-4.21 (m, 2H), 4.35-4.57 (m, 2H), 4.65-4.83 (m, 1H), 7.47-7.73 (m, 10H), 8.12-8.23 (m, 2H). Chiral separation condition: n-hexane/EtOH contained 0.1% DEA, 30/70; IC (4.6×250 mm, 5 μm); retention time: 9.46 minute.

Compound 94-1: LC-MS (ESI) m/z: 682 [M+H]⁺; ¹H-NMR (CD₃OD, 400 MHz): δ (ppm) 0.78-1.18 (m, 4H), 1.36-1.83 (m, 12H), 2.37-2.48 (m, 1H), 3.02-3.07 (m, 1H), 3.94-4.11 (m, 2H), 4.23-4.47 (m, 2H), 4.55-4.83 (m, 1H), 7.37-7.53 (m, 10H), 8.03-8.12 (m, 2H). Chiral separation condition: n-hexane/EtOH contained 0.1% DEA, 30/70; IC (4.6×250 mm, 5 μm); retention time: 8.10 minute.

### Example 95

### Synthesis of (R)—N-(3-(4-aminopiperidin-1-yl)-1-(4′-chloro-[1,1′-biphenyl]-4-yl)-1,1-difluoro-3-oxopropan-2-yl)-1-(cyclopentylmethyl)-1H-indazole-5-sulfonamide (Compound 95-1), and (S)—N-(3-(4-aminopiperidin-1-yl)-1-(4′-chloro-[1,1′-biphenyl]-4-yl)-1,1-difluoro-3-oxopropan-2-yl)-1-(cyclopentylmethyl)-1H-indazole-5-sulfonamide (Compound 95-2)

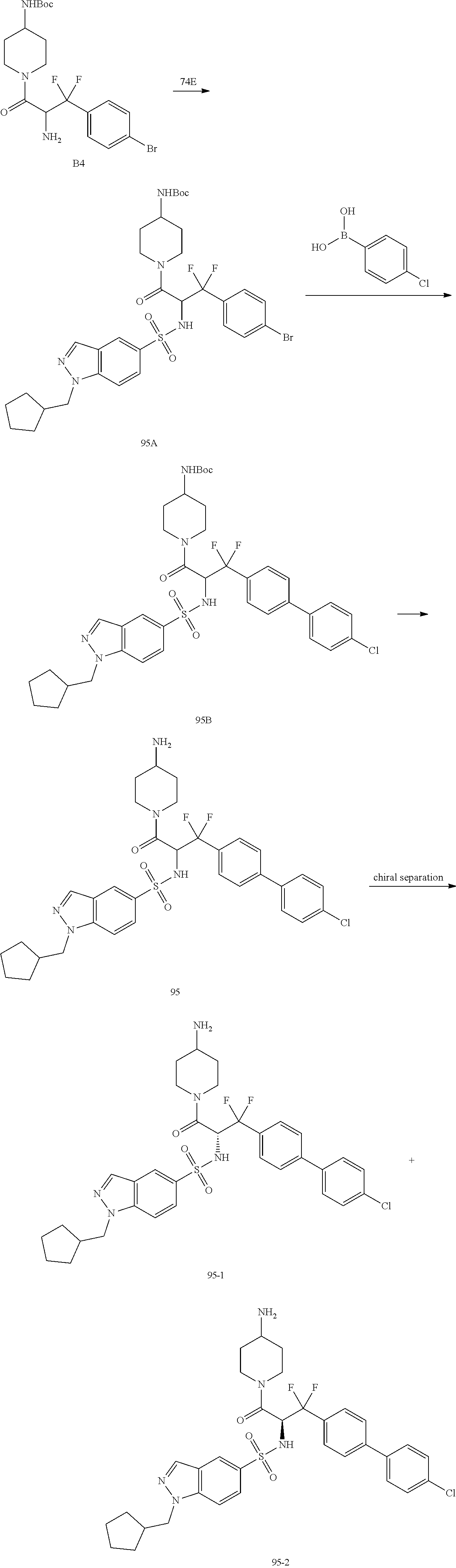

Compounds 95A, 95B, and 95 were synthesized by employing the procedures described for Compounds 2D, 14, and 1 using Compound B4, Compounds 74E, 4-chlorophenylboronic acid, 95A, and 95B in lieu of Compound B2, Compounds 2C, 14B, 14A, and 1A.

Compound 95A: LC-MS (ESI) m/z: 746 [M+Na]⁺; ¹H-NMR (CDCl₃, 400 MHz): δ (ppm) 0.81-1.46 (m, 8H), 1.57 (s, 9H), 1.72-2.01 (s, 5H), 2.50-2.60 (m, 2H), 2.82-2.99 (m, 1H), 3.43-3.56 (m, 1H), 4.05-4.25 (m, 1H), 4.34 (d, J=7.2 Hz, 2H), 4.63-4.78 (m, 1H), 5.77-5.98 (m, 1H), 7.22 (d, J=8.4 Hz, 2H), 7.42-7.47 (m, 3H), 7.56-7.65 (m, 1H), 8.09-8.18 (m, 2H).

Compound 95B: LC-MS (ESI) m/z: 778 [M+Na]⁺; ¹H-NMR (CDCl₃, 400 MHz): δ (ppm) 0.85-1.26 (m, 8H), 1.46 (s, 9H), 1.63-2.00 (m, 4H), 2.47-2.61 (m, 1H), 2.81-2.97 (m, 1H), 3.41-3.51 (m, 1H), 3.94-4.09 (m, 1H), 4.24 (d, J=7.6 Hz, 2H), 4.68-4.85 (m, 1H), 5.83-6.02 (m, 1H), 7.36-7.53 (m, 10H), 7.60-7.70 (m, 2H), 8.07-8.21 (m, 2H).

Compound 95 was separated with chiral HPLC to give Compound 95-1 and Compound 95-2.

Compound 95-1: LC-MS (ESI) m/z: 656 [M+H]⁺; ¹H-NMR (CD₃OD, 400 MHz): δ (ppm) 0.56-1.27 (m, 4H), 1.57-1.93 (m, 9H), 2.43-2.97 (m, 3H), 3.88-4.24 (m, 4H), 4.94-5.11 (m, 1H), 7.47-7.64 (m, 10H), 8.16-8.22 (m, 2H). Chiral separation condition: MeOH contained 0.1% NH₄OH; IC (4.6×250 mm, 5 μm); retention time: 7.02 minute.

Compound 95-2: LC-MS (ESI) m/z: 656 [M+H]⁺; ¹H-NMR (CD₃OD, 400 MHz): δ (ppm) 0.61-2.04 (m, 13H), 2.42-2.98 (m, 3H), 3.92-4.22 (m, 4H), 4.98-5.03 (m, 1H), 7.47-7.63 (m, 10H), 8.12-8.27 (m, 2H). Chiral separation condition: MeOH contained 0.1% NH₄OH; IC (4.6×250 mm, 5 μm); retention time: 5.41 minute.

### Example 96

### Synthesis of (R)—N-(3-(4-aminopiperidin-1-yl)-1-(4′-chloro-[1,1′-biphenyl]-4-yl)-1,1-difluoro-3-oxopropan-2-yl)-4-(cyclohexylmethoxy)benzenesulfonamide trifluoroacetate (Compound 96-1), and (S)—N-(3-(4-aminopiperidin-1-yl)-1-(4′-chloro-[1,1′-biphenyl]-4-yl)-1,1-difluoro-3-oxopropan-2-yl)-4-(cyclohexylmethoxy) benzenesulfonamide trifluoroacetate (Compound 96-2)

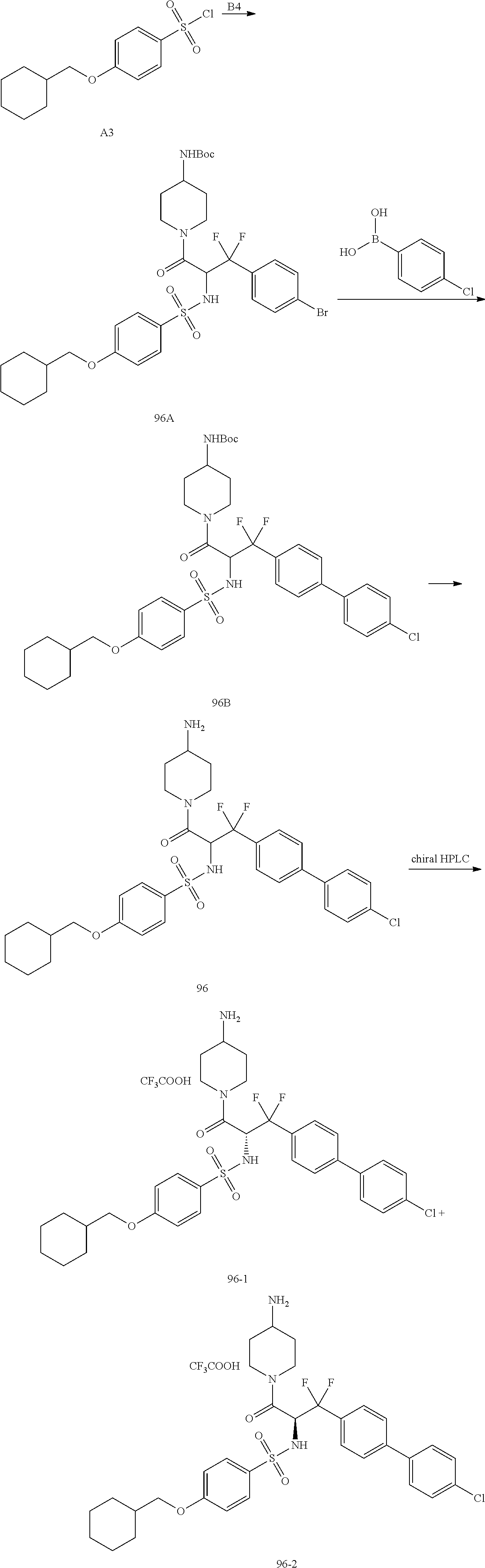

Compounds 96A, 96B, and 96 were synthesized by employing the procedures described for Compounds 2D, 14, and 1 using Compounds B4, A3, 4-chlorophenylboronic acid, Compounds 96A, and 96B in lieu of Compound B2, Compounds 2C, 14B, 14A, and 1A.

Compound 96A: LC-MS (ESI) m/z: 736 [M+Na]⁺.

Compound 96B: LC-MS (ESI) m/z: 768 [M+Na]⁺.

Compound 96 was separated with chiral HPLC to furnish Compound 96-1 and Compound 96-2.

Compound 96-1: LC-MS (ESI) m/z: 646 [M+H]⁺; ¹H-NMR (CD₃OD, 400 MHz): δ (ppm) 0.78-1.68 (m, 13H), 1.93-2.07 (m, 2H), 2.47-2.74 (m, 1H), 3.02-3.14 (m, 1H), 3.31-3.50 (m, 3H), 4.13-4.54 (m, 2H), 4.86-4.89 (m, 1H), 6.61-6.72 (m, 2H), 7.31-7.33 (m, 2H), 7.39-7.41 (m, 5H), 7.49-7.51 (m, 1H), 7.55-7.57 (m, 2H). Chiral separation condition: MeOH/Acetonitrile contained 0.5% NH₄OH, 1/1; IC (4.6×250 mm 5 μm); retention time: 4.11 minute.

Compound 96-2: LC-MS (ESI) m/z: 646 [M+H]⁺; ¹H-NMR (CD₃OD, 400 MHz): δ (ppm) 0.92-1.79 (m, 13H), 2.03-2.17 (m, 2H), 2.56-2.85 (m, 1H), 3.17-3.28 (m, 1H), 3.38-3.61 (m, 3H), 4.23-4.66 (m, 2H), 4.95-4.99 (m, 1H), 6.72-6.83 (m, 2H), 7.42-7.44 (m, 2H), 7.49-7.54 (m, 5H), 7.60-7.62 (m, 1H), 7.66-7.69 (m, 2H). Chiral separation condition: MeOH/Acetonitrile contained 0.5% NH₄OH, 1/1; IC (4.6×250 mm 5 μm); retention time: 3.12 minute.

### Example 97

### Synthesis of (R)—N-(3-(4-aminopiperidin-1-yl)-1-(4-(cyclohexyloxy)phenyl)-1,1-difluoro-3-oxopropan-2-yl)-6-(cyclopentyloxy)naphthalene-2-sulfonamide (Compound 97-1), and (S)—N-(3-(4-aminopiperidin-1-yl)-1-(4-(cyclohexyloxy) phenyl)-1,1-difluoro-3-oxopropan-2-yl)-6-(cyclopentyloxy)naphthalene-2-sulfonamide (Compound 97-2)

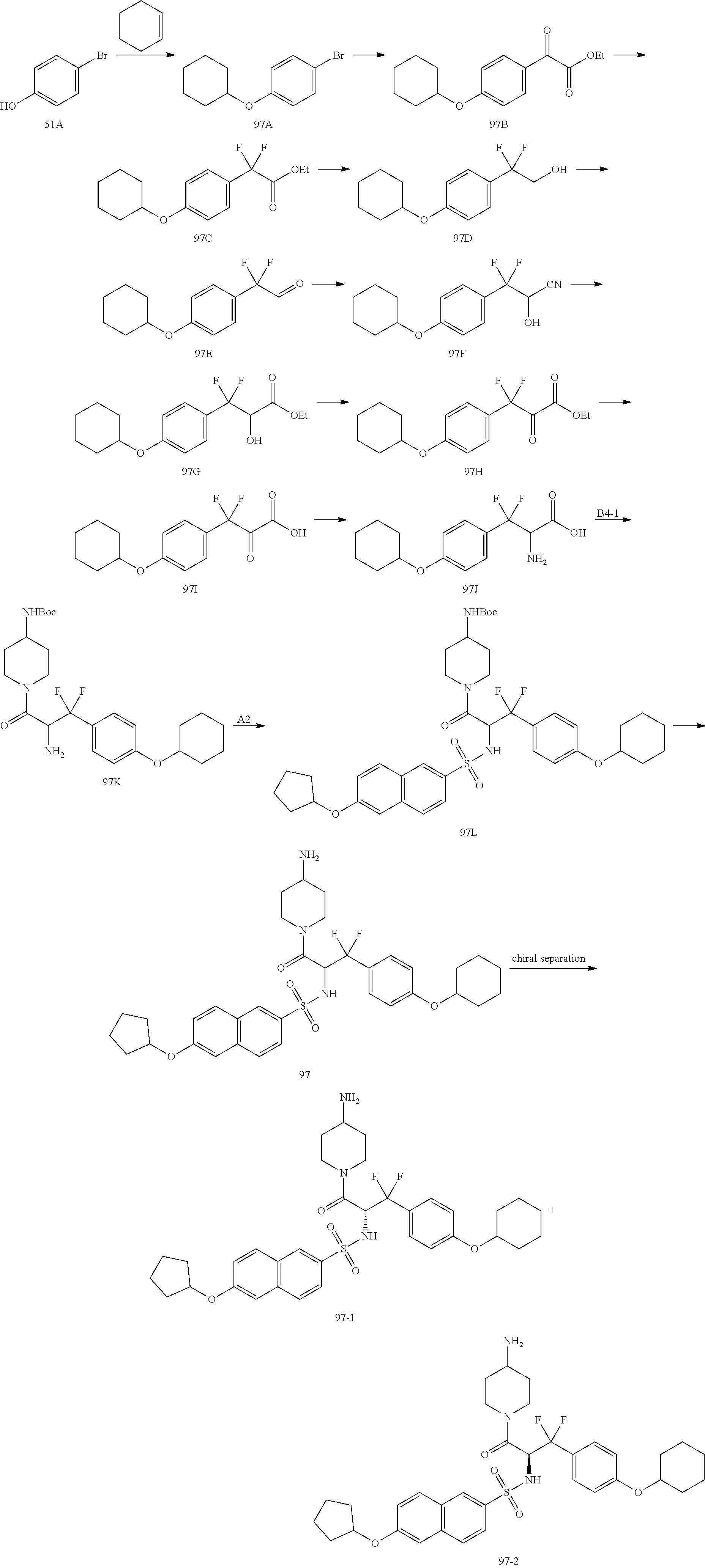

A solution of Compound 51A (50 g, 290 mmol) and cyclohexene (36 g, 435 mmol) in dichloromethane (500 mL) was added a solution of BF₃Et₂O in diethyl ether (3.0 M, 290 mL) at 0° C. The reaction mixture was stirred at room temperature for 16 hours, treated with water (200 mL), and extracted with dichloromethane (200 mL×2). The combined organic extracts were washed with water (200 mL×4) and brine (100 mL), dried over anhydrous sodium sulfate, filtered, and concentrated. The residue was purified by silica gel column chromatography to furnish Compound 97A. ¹H-NMR (CD₃OD, 400 MHz): δ (ppm) 1.31-1.48 (m, 6H), 1.71-1.72 (m, 2H), 1.92-1.99 (m, 2H), 4.36-4.49 (m, 1H), 6.93 (d, J=2.8 Hz, 2H), 7.95 (d, J=7.2 Hz, 2H).

Compounds 97B, 97C, 97D, 97E, 97F, 97G, 97H, 97I, 97J, 97K, 97L, and 97 were synthesized by employing the procedures described for Compound 9B, Compound B2-3, Compound 51E, Compound B6-3, Compounds 45F, 45G, Compound B6-3, Compounds 451, 80A, Compound B4, Compounds 2D, and 1 using Compounds 97A, 97B, 97C, 97D, 97E, 97F, 97G, 97H, 97I, 97J, 97K, Compound A2, and 97L in lieu of Compound 9A, Compound B2-2, Compound 51D, Compound B6-2, Compounds 45E, 45F, Compound B6-2, Compounds 45H, 51J, Compounds B2-6, B2, Compounds 2C, and 1A.

Compound 97B: ¹H-NMR (CD₃OD, 400 MHz): δ (ppm) 1.27-1.46 (m, 6H), 1.52-1.63 (m, 3H), 1.81-1.86 (m, 2H), 1.95-1.98 (m, 2H), 4.36-4.49 (m, 3H), 6.94 (d, J=2.8 Hz, 2H), 7.97 (d, J=7.2 Hz, 2H).

Compound 97C: LC-MS (ESI) m/z: 299 [M+H]⁺; ¹H-NMR (CDCl₃, 400 MHz): δ (ppm) 1.24-1.43 (m, 6H), 1.49-1.60 (m, 3H), 1.79-1.84 (m, 2H), 1.95-1.99 (m, 2H), 4.24-4.32 (m, 3H), 6.92 (d, J=8.8 Hz, 2H), 7.597 (d, J=8.8 Hz, 2H).

Compound 97D: LC-MS (ESI) m/z: 236 [M−18]⁺; ¹H-NMR (CD₃OD, 400 MHz): δ (ppm) 1.32-1.64 (m, 6H), 1.79-1.82 (m, 2H), 1.97-1.99 (m, 2H), 3.86 (t, J=13.6 Hz, 2H), 4.34-4.40 (m, 1H), 6.97 (d, J=9.2 Hz, 2H), 7.42 (d, J=8.4 Hz, 2H).

Compound 97E was used directly for the next step without further purification. ¹H-NMR (CDCl₃, 400 MHz): δ (ppm) 1.28-1.61 (m, 6H), 1.71-1.78 (m, 2H), 1.87-1.92 (m, 2H), 4.31-4.42 (m, 1H), 6.87 (d, J=8.8 Hz, 2H), 7.35 (d, J=8.4 Hz, 2H).

Compound 97F: LC-MS (ESI) m/z: 277 [M+Na]⁺; ¹H-NMR (CDCl₃, 400 MHz): δ (ppm) δ (ppm) 0.90-1.90 (m, 8H), 1.93-2.07 (m, 2H), 4.30-4.36 (m, 1H), 4.77-4.83 (m, 1H), 6.98 (d, J=8.8 Hz, 2H), 7.48 (d, J=8.8 Hz, 2H).

Compound 97G: LC-MS (ESI) m/z: 329 [M+H]⁺; ¹H-NMR (CDCl₃, 400 MHz): δ (ppm) 0.90-1.90 (m, 11H), 1.91-2.03 (m, 2H), 4.23-4.36 (m, 3H), 4.47-4.65 (m, 1H), 6.88 (d, J=8.8 Hz, 2H), 7.38 (d, J=8.4 Hz, 2H).

Compound 97H: LC-MS (ESI) m/z: No; ¹H-NMR (CD₃OD, 400 MHz): δ (ppm) 1.18-1.61 (m, 9H), 1.80-1.85 (m, 2H), 1.88-2.06 (m, 2H), 4.26-4.44 (m, 3H), 6.89-6.97 (m, 2H), 7.51 (d, J=7.2 Hz, 2H).

Compound 971: LC-MS (ESI) m/z: 297 [M−H]⁺.

Compound 97J: LC-MS (ESI) m/z: 336 [M+H]⁺; ¹H-NMR (CD₃OD, 400 MHz): 1.20-1.61 (m, 6H), 1.70-1.72 (m, 2H), 1.88-1.92 (m, 2H), 4.04-4.44 (m, 1H), 4.83-4.90 (m, 1H), 7.05 (d, J=9.2 Hz, 2H), 7.46 (d, J=9.2 Hz, 2H).

Compound 97K: LC-MS (ESI) m/z: 482 [M+H]⁺; ¹H-NMR (CD₃OD, 400 MHz): δ (ppm) 0.67-1.61 (m, 14H), 1.81-2.05 (m, 9H), 2.62-3.09 (m, 2H), 3.48-3.79 (m, 2H), 4.12-4.58 (m, 3H), 6.85-6.93 (m, 2H), 7.30-7.36 (m, 2H).

Compound 97L: LC-MS (ESI) m/z: 700 [M−55]⁺.

Compound 97 was separated with chiral HPLC to furnish Compound 97-1 and Compound 97-2.

Compound 97-1: LC-MS (ESI) m/z: 656 [M+H]⁺; ¹H-NMR (CD₃OD, 400 MHz): δ (ppm) 0.98-2.11 (m, 23H), 2.87-3.12 (m, 2H), 4.03-4.40 (m, 3H), 4.95-5.02 (m, 2H), 6.69-6.84 (m, 2H), 7.12-7.36 (m, 4H), 7.57-7.61 (m, 1H), 7.76-7.89 (m, 2H), 8.13-8.17 (m, 1H). Chiral separation condition: MeOH contained 0.1% NH₄OH; (R,R)-Whelk-O1 (4.6×250 mm, 5 μm); retention time: 5.36 minute.

Compound 97-2: LC-MS (ESI) m/z: 656 [M+H]⁺; ¹H-NMR (CD₃OD, 400 MHz): δ (ppm) 1.24-2.12 (m, 23H), 2.69-3.22 (m, 2H), 4.10-4.55 (m, 3H), 4.96-5.08 (m, 2H), 6.61-6.82 (m, 2H), 7.20-7.36 (m, 4H), 7.52-7.60 (m, 1H), 7.73-7.88 (m, 2H), 8.08-8.16 (m, 1H). Chiral separation condition: MeOH contained 0.1% NH₄OH; (R,R)-Whelk-O1 (4.6×250 mm, 5 μm); retention time: 6.64 minute.

### Example 98

### Synthesis of (R)—N-(3-(4-aminopiperidin-1-yl)-1-(4-chlorophenyl)-1,1-difluoro-3-oxopropan-2-yl)-6-(cyclopentyloxy)naphthalene-2-sulfonamide (Compound 98-1), and (S)—N-(3-(4-aminopiperidin-1-yl)-1-(4-chlorophenyl)-1,1-difluoro-3-oxopropan-2-yl)-6-(cyclopentyloxy)naphthalene-2-sulfonamide (Compound 98-2)

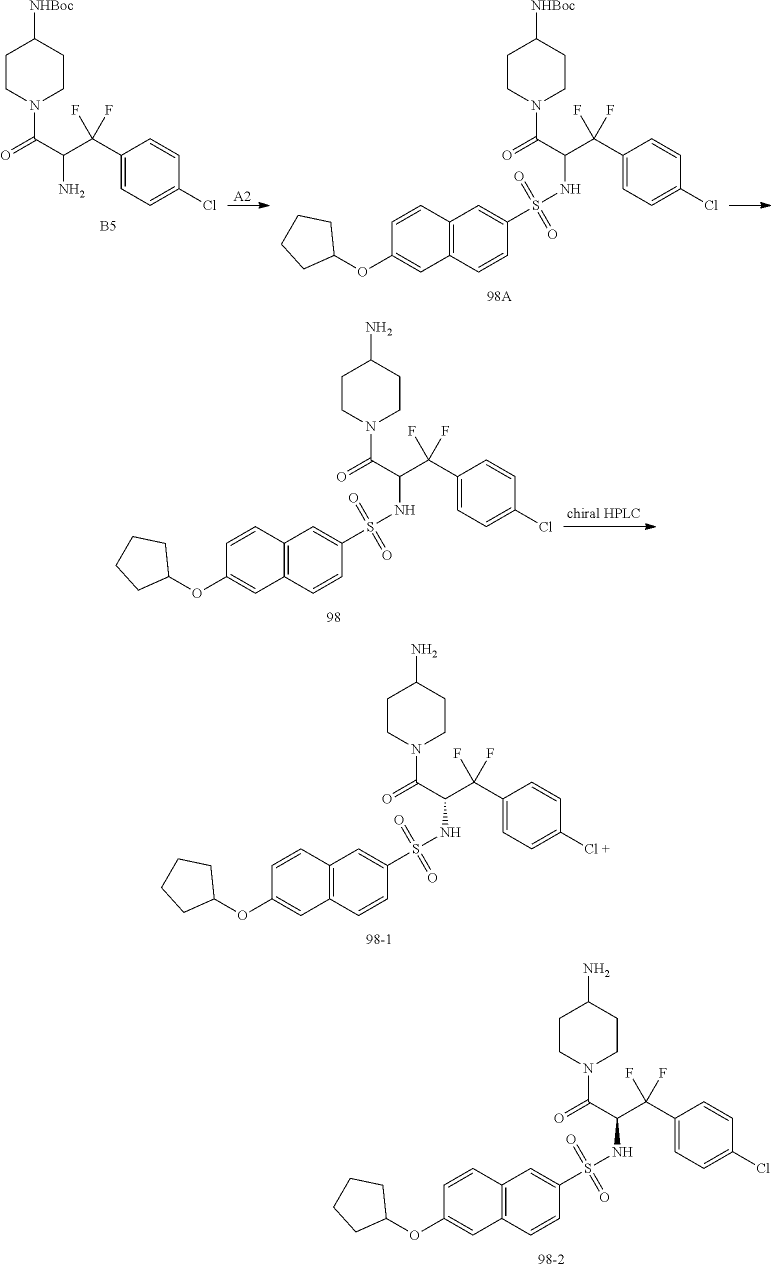

Compounds 98A and 98 were synthesized by employing the procedures described for Compounds 2D and 1 using Compounds B5, A2, and Compound 98A in lieu of Compound B2, Compounds 2C, and 1A.

Compound 98A: LC-MS (ESI) m/z: 714 [M+Na]⁺.

Compound 98 was separated with chiral HPLC (Chiral separation condition: MeOH contained 0.5% NH₄₀H; IC, 250×4.6 mm, 5 μm) to give Compound 98-1 and Compound 98-2.

Compound 98-1: LC-MS (ESI) m/z: 592 [M+H]⁺; ¹H-NMR (CDCl₃ and CD₃OD, 400 MHz): δ (ppm) 0.79-1.00 (m, 1H), 1.61 (s, 2H), 1.77-1.95 (m, 9H), 2.55-2.80 (m, 1H), 2.95-3.04 (m, 2H), 3.85 (s, 1H), 4.08-4.40 (m, 1H), 4.70-4.73 (m, 1H), 4.87 (s, 1H), 7.07-7.26 (m, 6H), 7.38-7.44 (m, 1H), 7.63 (d, J=8.4 Hz, 1H), 7.70-7.73 (m, 1H), 8.03 (s, 1H). Chiral separation condition: MeOH contained 0.5% NH₄OH; IC (4.6×250 mm, 5 μm); retention time: 4.28 minute.

Compound 98-2: LC-MS (ESI) m/z: 592 [M+H]⁺; ¹H-NMR (CDCl₃, 400 MHz): δ (ppm) 0.98-1.10 (m, 1H), 1.46-1.56 (m, 2H), 1.79-2.01 (m, 7H), 2.11-2.24 (m, 1H), 2.42-2.55 (m, 2H), 2.66-2.94 (m, 2H), 3.58-3.72 (m, 1H), 3.92-4.15 (m, 1H), 4.60-4.77 (m, 1H), 4.87-4.95 (m, 1H), 7.12 (d, J=8.4 Hz, 1H), 7.18-7.23 (m, 1H), 7.28-7.33 (m, 4H), 7.55-7.80 (m, 3H), 8.17 (d, J=20 Hz, 1H). Chiral separation condition: MeOH contained 0.5% NH₄OH; IC (4.6×250 mm, 5 μm); retention time: 3.56 minute.

### Example 99

### Synthesis of (R)—N-(3-(4-aminopiperidin-1-yl)-1-(4-chlorophenyl)-1,1-difluoro-3-oxopropan-2-yl)-1-(cyclopentylmethyl)-1H-indazole-5-sulfonamide (Compound 99-1), and (S)—N-(3-(4-aminopiperidin-1-yl)-1-(4-chlorophenyl)-1,1-difluoro-3-oxopropan-2-yl)-1-(cyclopentylmethyl)-1H-indazole-5-sulfonamide (Compound 99-2)

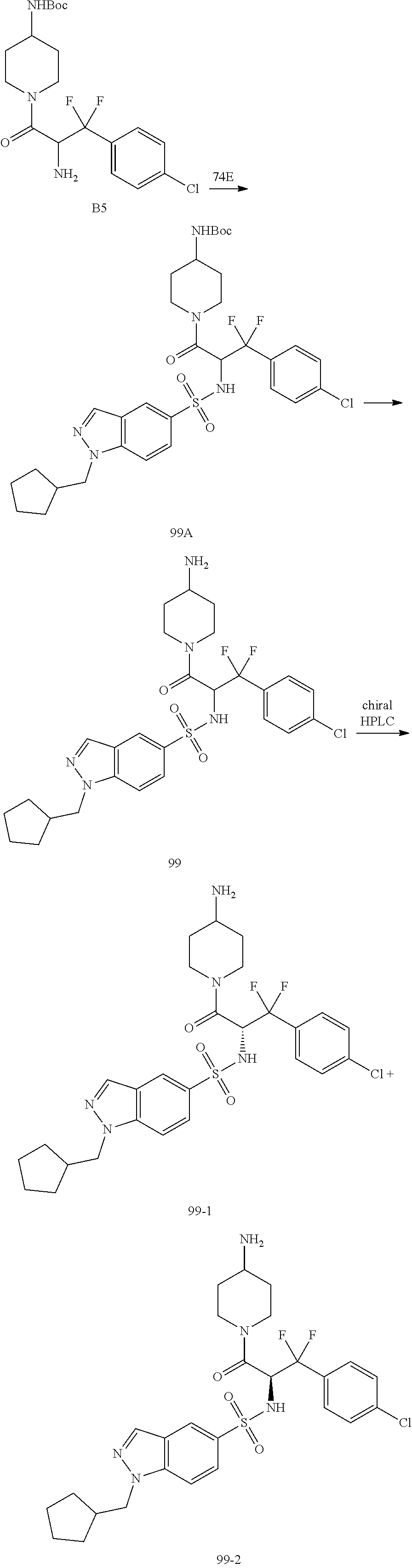

Compounds 99A and 99 were synthesized by employing the procedures described for Compounds 1A and 1 using Compound B5, Compounds 74E, and 99A in lieu of Compounds B2, A1, and Compound 1A.

Compound 99A: LC-MS (ESI) m/z: 624 [M−55]⁺.

Compound 99 was separated with chiral HPLC (Chiral separation condition: MeOH contained 0.5% NH₄OH; IC, 250×4.6 mm, 5 m) to give Compound 99-1 and Compound 99-2.

Compound 99-1: LC-MS (ESI) m/z: 580 [M+H]⁺; ¹H-NMR (CDCl₃ and CD₃OD, 400 MHz): δ (ppm) 1.19-1.23 (m, 4H), 1.37-1.51 (m, 4H), 1.87-2.23 (m, 3H), 2.47 (s, 1H), 2.66-3.31 (m, 4H), 3.94-4.02 (m, 1H), 4.27-4.51 (m, 3H), 4.75-4.79 (m, 1H), 7.01-7.45 (m, 6H), 7.99-8.06 (m, 2H). Chiral separation condition: MeOH contained 0.5% NH₄OH; IC (250×4.6 mm, 5 μm); retention time: 3.96 minute.

Compound 99-2: LC-MS (ESI) m/z: 580 [M+H]⁺; ¹H-NMR (CDCl₃, 400 MHz): δ (ppm) 0.27-1.18 (m, 4H), 1.25-1.31 (m, 5H), 1.98-2.26 (m, 3H), 2.50-2.97 (m, 4H), 3.67-3.78 (m, 1H), 3.99-4.11 (m, 1H), 4.32 (d, J=7.6 Hz, 2H), 4.69-4.79 (m, 1H), 7.29-7.31 (m, 4H), 7.42-7.48 (m, 1H), 7.59-7.64 (m, 1H), 8.09-8.18 (m, 2H). Chiral separation condition: MeOH contained 0.5% NH₄OH; IC (250×4.6 mm, 5 μm); retention time: 3.21 minute.

### Example 100

### Synthesis of (R)—N-(3-(4-aminopiperidin-1-yl)-1-(4-chlorophenyl)-1,1-difluoro-3-oxopropan-2-yl)-6-(cyclopentyloxy)-N-methylnaphthalene-2-sulfonamide (Compound 100-1), and (S)—N-(3-(4-aminopiperidin-1-yl)-1-(4-chlorophenyl)-1,1-difluoro-3-oxopropan-2-yl)-6-(cyclopentyloxy)-N-methylnaphthalene-2-sulfonamide (Compound 100-2)

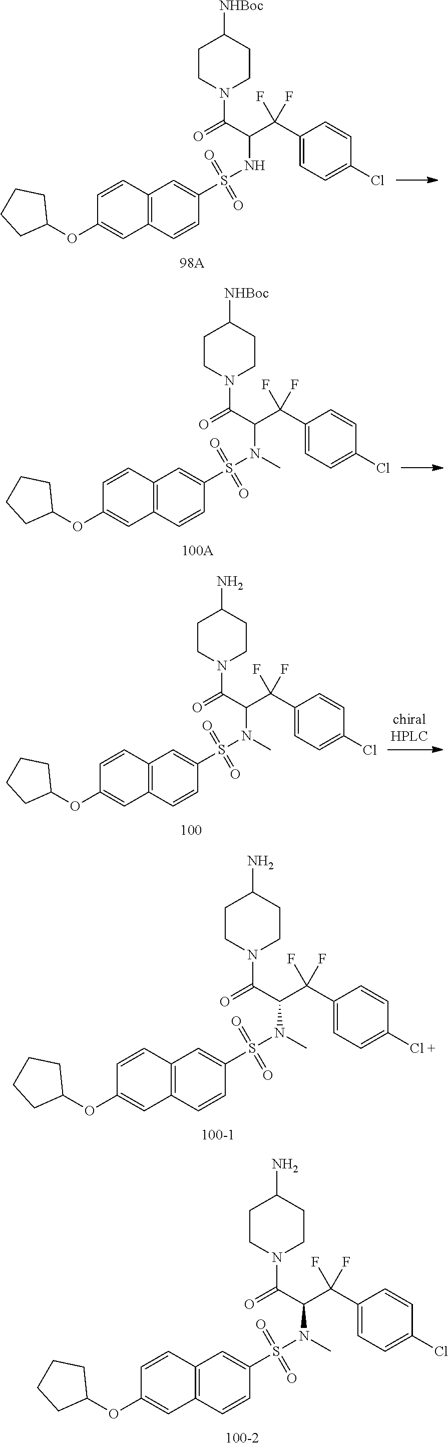

Compounds 100A and 100 were synthesized by employing the procedures described for Compounds 34A and 1 using Compounds 98A and 100A in lieu of Compounds 5A and 1A.

Compound 100A: LC-MS (ESI) m/z: 728 [M+Na]⁺.

Compound 100 was separated with chiral HPLC (Chiral separation condition: MeOH contained 0.5% NH₄OH; IC, 250×4.6 mm; 5 m) to give Compound 100-1 and Compound 100-2.

Compound 100-1: LC-MS (ESI) m/z: 606 [M+H]⁺; ¹H-NMR (CDCl₃ and CD₃OD, 400 MHz): δ (ppm) 1.28-2.15 (m, 12H), 2.52 (s, 1H), 2.89-3.27 (m, 5H), 4.10-4.18 (m, 1H), 4.33-4.50 (m, 1H), 4.85 (s, 1H), 5.25-5.62 (m, 1H), 7.08-7.46 (m, 7H), 7.64-7.76 (s, 2H), 8.09 (d, J=21.6 Hz, 1H). Chiral separation condition: MeOH (0.5% NH₄OH); IC (4.6×250 mm, 5 μm); retention time: 3.4 minute.

Compound 100-2: LC-MS (ESI) m/z: 606 [M+H]⁺; ¹H-NMR (CDCl₃, 400 MHz): δ (ppm) 1.62-2.04 (m, 12H), 2.61 (t, J=12.8 Hz, 1H), 2.84 (s, 1H), 2.93-3.23 (m, 4H), 3.97-4.35 (m, 2H), 4.89-4.93 (m, 1H), 5.32-5.71 (m, 1H), 7.14 (s, 1H), 7.19-7.22 (m, 1H), 7.30-7.43 (m, 4H), 7.63 (t, J=8.0 Hz, 1H), 7.75-7.83 (m, 2H), 8.24 (d, J=6 Hz, 1H). Chiral separation condition: MeOH (0.5% NH₄OH); IC (4.6×250 mm, 5 μm); retention time: 2.7 minute.

### Example 101

### Synthesis of N-((2R)-3-(3-amino-8-azabicyclo[3.2.1]octan-8-yl)-1-(4-(5-chlorothiophen-2-yl)phenyl)-1,1-difluoro-3-oxopropan-2-yl)-6-(cyclopentyloxy)naphthalene-2-sulfonamide (Compound 101-1), and N-((2S)-3-(3-amino-8-azabicyclo[3.2.1]octan-8-yl)-1-(4-(5-chlorothiophen-2-yl)phenyl)-1,1-difluoro-3-oxopropan-2-yl)-6-(cyclopentyloxy)naphthalene-2-sulfonamide (Compound 101-2)

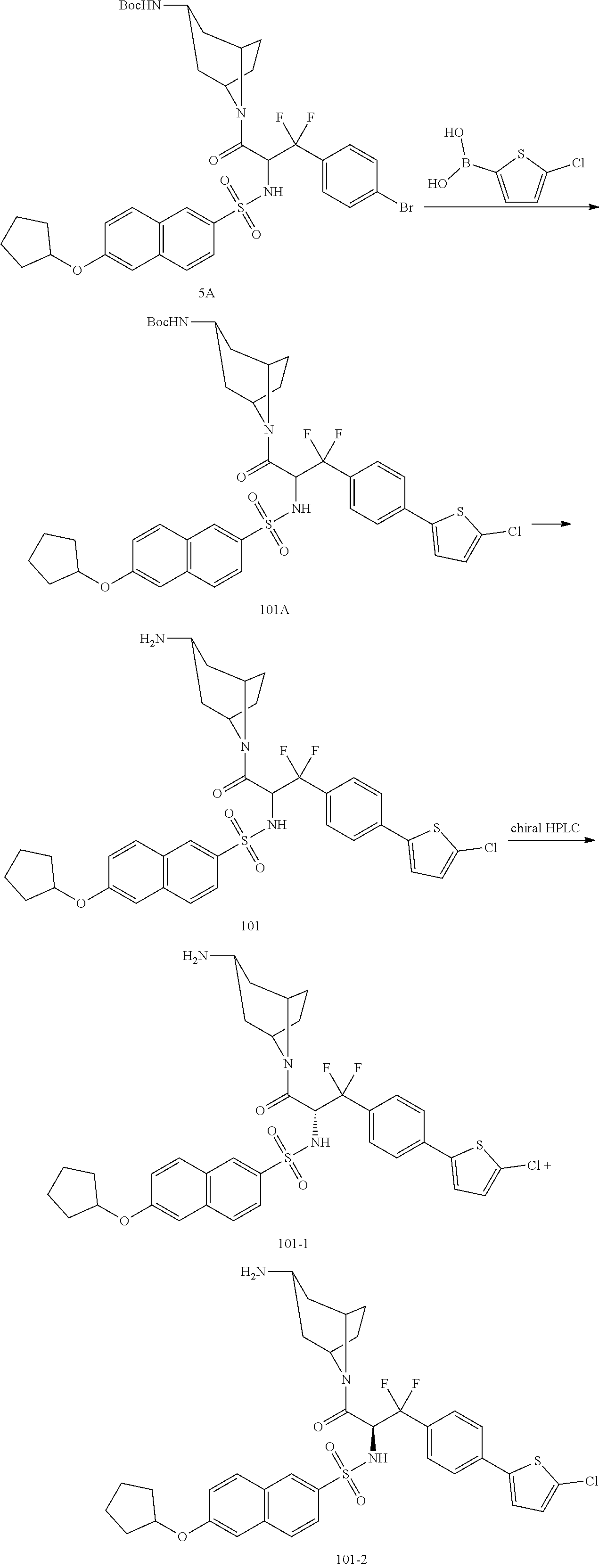

Compounds 101A and 101 were synthesized by employing the procedures described for Compounds 14 and 1 using 5-chlorothiophen-2-ylboronic acid, Compounds 5A, and 101A in lieu of Compounds 14B, 14A, and 1A.

Compound 101A: LC-MS: (ESI) m/z: 744 [M−55]⁺.

Compound 101 was separated with chiral HPLC to give Compound 101-1 and Compound 101-2.

Compound 101-1: LC-MS (ESI) m/z: 700 [M+H]⁺; ¹H-NMR (CD₃OD, 400 MHz): δ (ppm) 0.76-0.85 (m, 1H), 1.12-1.46 (m, 3H), 1.64-2.09 (m, 12H), 2.87-3.07 (m, 1H), 4.26-4.47 (m, 2H), 4.71-4.77 (m, 1H), 6.99-7.01 (m, 2H), 7.14-7.24 (m, 3H), 7.42-7.46 (m, 4H), 7.57-7.85 (m, 3H), 8.14-8.18 (m, 1H). Chiral separation condition: MeOH/MeCN contained 0.5% NH₄OH, 1/1; (R,R)-whelk-O1 (4.6×250 mm, 5 μm); retention time: 4.59 minute.

Compound 101-2: LC-MS (ESI) m/z: 700 [M+H]⁺; ¹H-NMR (CD₃OD, 400 MHz): δ (ppm) 0.59-0.69 (m, 1H), 0.83-1.30 (m, 3H), 1.45-1.93 (m, 12H), 2.74-2.89 (m, 1H), 4.12-4.32 (m, 2H), 4.51-4.61 (m, 1H), 4.77-4.79 (m, 1H), 6.85-6.87 (m, 1H), 6.99-7.09 (m, 3H), 7.28-7.31 (m, 4H), 7.42-7.70 (m, 3H), 7.99-8.03 (m, 1H). Chiral separation condition: MeOH/MeCN contained 0.5% NH₄OH, 1/1; (R,R)-whelk-O1 (4.6×250 mm, 5 μm); retention time: 5.92 minute.

### Example 102

### Synthesis of N-((2R)-3-(3-amino-8-azabicyclo[3.2.1]octan-8-yl)-1-(4-(cyclohexyloxy)phenyl)-1,1-difluoro-3-oxopropan-2-yl)-6-(cyclopentyloxy) naphthalene-2-sulfonamide (Compound 102-1), and N-((2S)-3-(3-amino-8-azabicyclo[3.2.1]octan-8-yl)-1-(4-(cyclohexyloxy)phenyl)-1,1-difluoro-3-oxopropan-2-yl)-6-(cyclopentyloxy)naphthalene-2-sulfonamide (Compound 102-2)

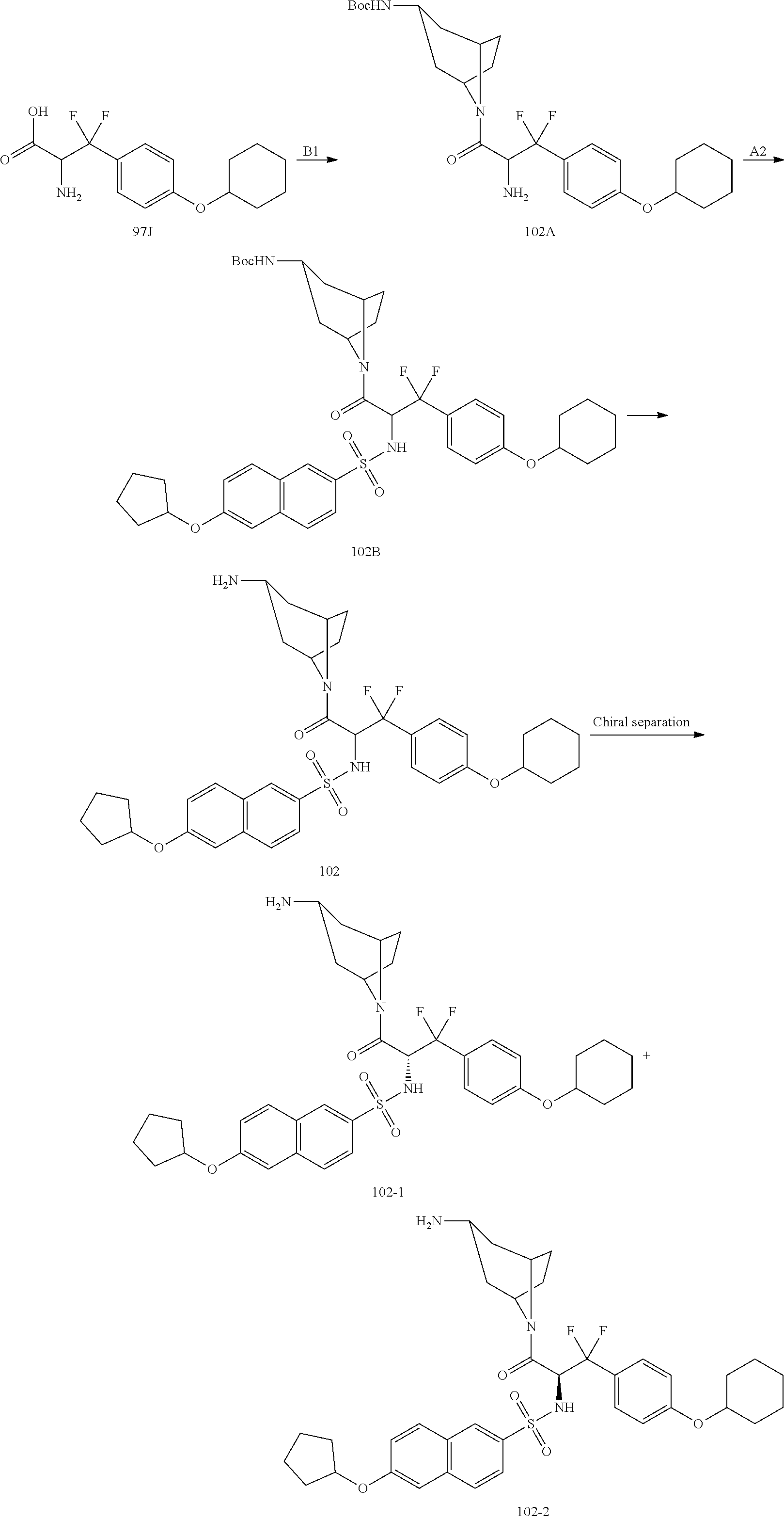

Compounds 102A, 102B, and 102 were synthesized by employing the procedures described for Compound B4, Compounds 2D, and 1 using Compound 97J, Compounds B1, A2, Compounds 102A, and 102B in lieu of Compounds B2-6, B4-1, Compound 2C, Compound B2, and Compound 1A.

Compound 102A: LC-MS (ESI) m/z: 508 [M+H]⁺.

Compound 102B: LC-MS (ESI) m/z: 726 [M−55]⁺.

Compound 102 was separated with chiral HPLC to furnish Compound 102-1 and Compound 102-2.

Compound 102-1: LC-MS (ESI) m/z: 682 [M+H]⁺; ¹H-NMR (CD₃OD, 400 MHz): δ (ppm) 0.30-0.46 (m, 1H), 0.97-1.97 (m, 25H), 2.61-2.84 (m, 1H), 3.90-4.24 (m, 3H), 4.50-4.59 (m, 1H), 4.86-4.89 (m, 1H), 6.74-6.79 (m, 2H), 7.09-7.30 (m, 4H), 7.55-7.81 (m, 3H), 8.10-8.18 (m, 1H). Chiral separation condition: MeOH contained 0.5% NH₄OH; IC (250×4.6 mm, 5 μm); retention time: 6.6 minute.

Compound 102-2: LC-MS (ESI) m/z: 682 [M+H]⁺; ¹H-NMR (CD₃OD, 400 MHz): δ (ppm) 0.33-0.48 (m, 1H), 1.19-1.97 (m, 25H), 2.63-2.84 (m, 1H), 3.91-4.24 (m, 3H), 4.50-4.59 (m, 1H), 4.86-4.89 (m, 1H), 6.74-6.79 (m, 2H), 7.09-7.30 (m, 4H), 7.55-7.80 (m, 3H), 8.10-8.18 (m, 1H). Chiral separation condition: MeOH contained 0.5% NH₄OH; IC (250×4.6 mm, 5 μm); retention time: 5.06 minute.

### Example 103

### Synthesis of (R)—N-(3-(4-aminopiperidin-1-yl)-1-(4-(cyclohexyloxy)phenyl)-1,1-difluoro-3-oxopropan-2-yl)-4-(cyclohexylmethoxy)benzenesulfonamide (Compound 103-1), and (S)—N-(3-(4-aminopiperidin-1-yl)-1-(4-(cyclohexyloxy)phenyl)-1,1-difluoro-3-oxopropan-2-yl)-4-(cyclohexylmethoxy)benzenesulfonamide (Compound 103-2)

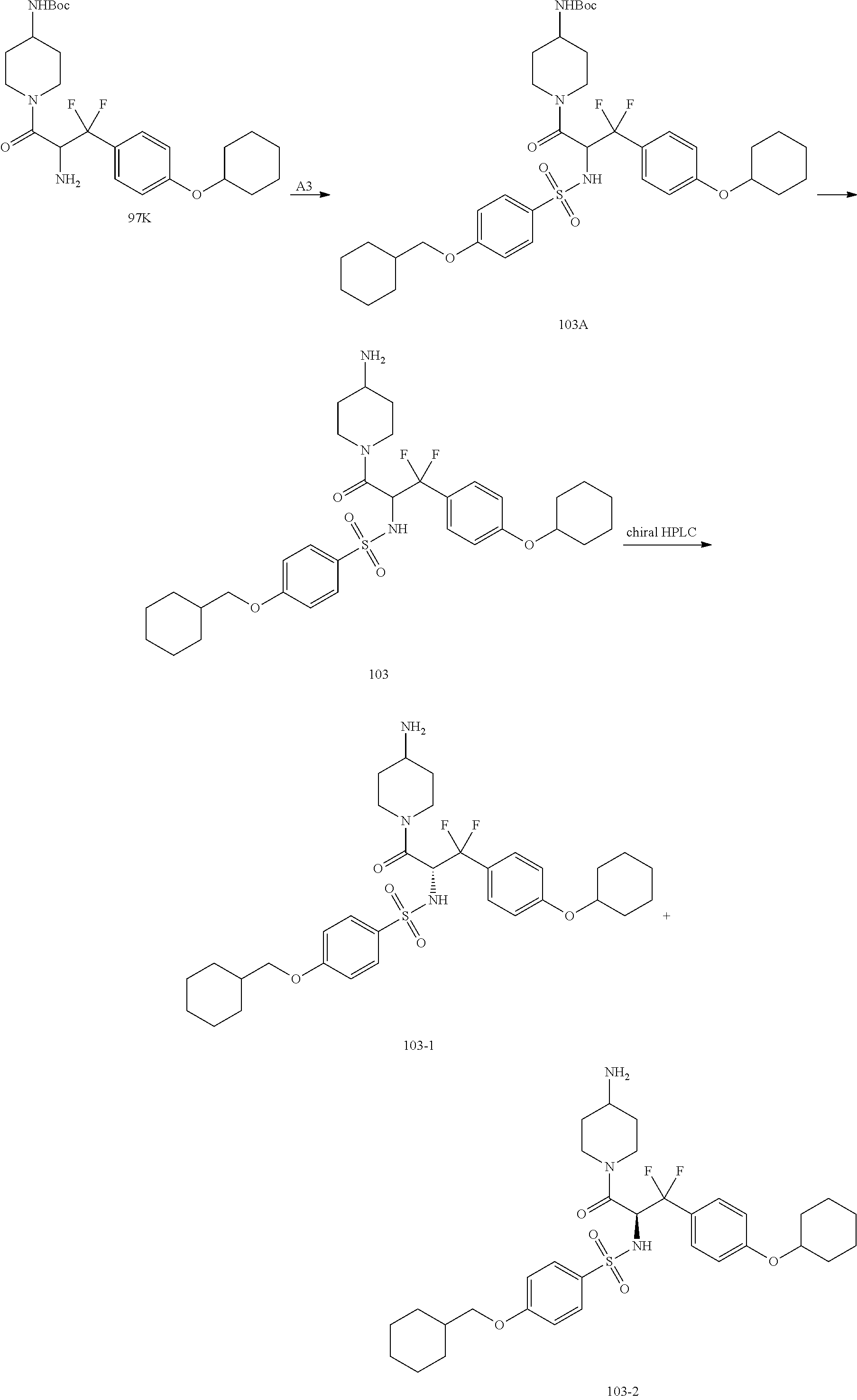

Compounds 103A and 103 were synthesized by employing the procedures 103A in lieu of Compound B2, Compounds 2C, and 1A.

Compound 103A: LC-MS (ESI) m/z: 678 [M−55]f.

Compound 103 was separated with chiral HPLC to furnish Compound 103-1 and Compound 103-2.

Compound 103-1: LC-MS (ESI) m/z: 634 [M+H]⁺; ¹H-NMR (CD₃OD, 400 MHz): δ (ppm) 0.96-1.99 (m, 26H), 2.30-2.64 (m, 1H), 2.88-3.02 (m, 1H), 3.25-3.28 (m, 1H), 3.46-3.75 (m, 3H), 4.00-4.41 (m, 3H), 6.72-6.84 (m, 4H), 7.15-7.27 (m, 2H), 7.41-7.47 (m, 2H). Chiral separation condition: MeOH contained 0.5% NH₄OH; IC (250×4.6 mm, 5 μm); retention time: 4.35 minute.

Compound 103-2: LC-MS (ESI) m/z: 634 [M+H]⁺; ¹H-NMR (CD₃OD, 400 MHz): δ (ppm) 0.85-2.02 (m, 26H), 2.29-2.92 (m, 3H), 3.78-3.86 (m, 3H), 4.07-4.19 (m, 1H), 4.36-4.40 (m, 1H), 4.78-4.86 (m, 1H), 6.90-7.00 (m, 4H), 7.31-7.37 (m, 2H), 7.61-7.65 (m, 2H). Chiral separation condition: MeOH contained 0.5% NH₄OH; IC (250×4.6 mm, 5 μm); retention time: 3.43 minute.

### Example 104

### Synthesis of N-((2R)-3-(3-amino-8-azabicyclo[3.2.1]octan-8-yl)-1-(4-(5-chlorothiophen-2-yl)phenyl)-1,1-difluoro-3-oxopropan-2-yl)-4-(cyclohexylmethoxy) benzenesulfonamide (Compound 104-1), and N-((2S)-3-(3-amino-8-azabicyclo[3.2.1]octan-8-yl)-1-(4-(5-chlorothiophen-2-yl)phenyl)-1,1-difluoro-3-oxopropan-2-yl)-4-(cyclohexylmethoxy)benzenesulfonamide (Compound 104-2)

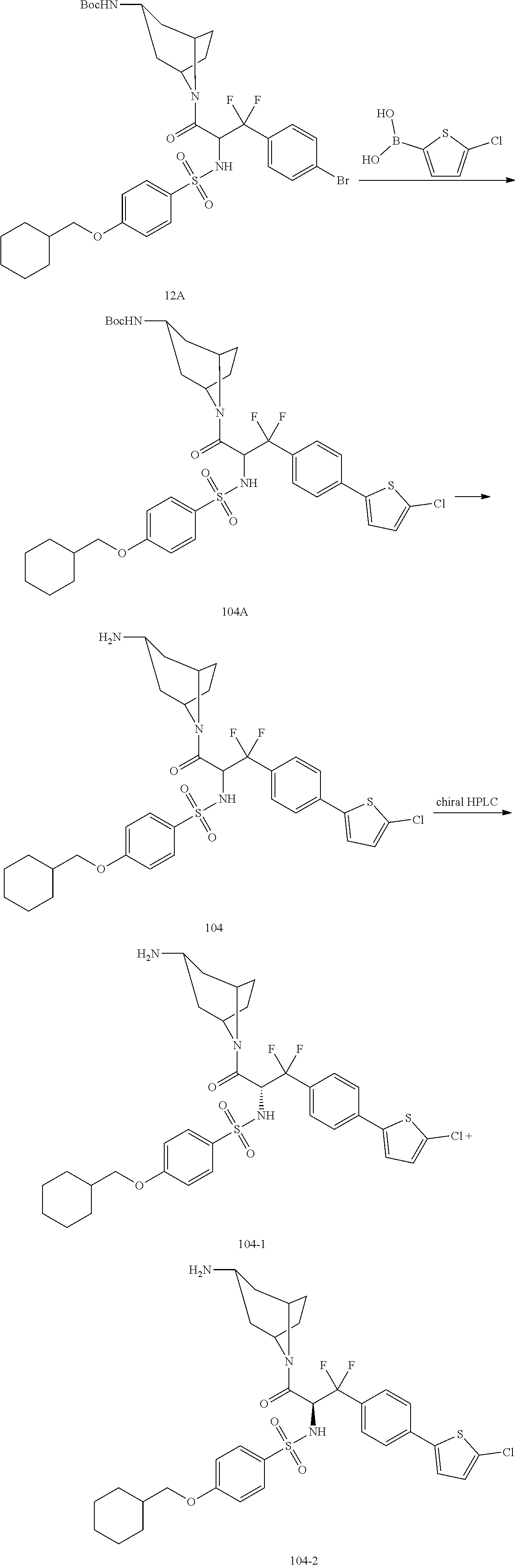

Compounds 104A and 104 were synthesized by employing the procedures described for Compounds 14 and 1 using 5-chlorothiophen-2-ylboronic acid, Compounds 12A, and 104A in lieu of Compounds 14B, 14A, and 1A.

Compound 104A: LC-MS: (ESI) m/z: 778 [M+H]⁺.

Compound 104 was separated with chiral HPLC to give Compound 104-1 and Compound 104-2

Compound 104-1: LC-MS (ESI) m/z: 678 [M+H]⁺; ¹H-NMR (CD₃OD, 400 MHz): δ (ppm) 1.00-1.34 (m, 8H), 1.71-1.84 (m, 11H), 3.03-3.14 (m, 1H), 3.65-3.7 (m, 2H), 4.30-4.68 (m, 3H), 6.85-6.88 (m, 2H), 7.02 (d, J=4.0 Hz, 1H), 7.31-7.34 (m, 1H), 7.40-7.43 (m, 1H), 7.47-7.59 (m, 5H). Chiral separation condition: MeOH contained 0.5% NH₄OH; IC (4.6×150 mm, 5 μm); retention time: 7.57 minute.

Compound 104-2: LC-MS (ESI) m/z: 678 [M+H]⁺; ¹H-NMR (CD₃OD, 400 MHz): δ (ppm) 0.88-1.34 (m, 8H), 1.69-1.84 (m, 11H), 3.05-3.13 (m, 1H), 3.64-3.70 (m, 2H), 4.30-4.68 (m, 3H), 6.85-6.88 (m, 2H), 7.02 (d, J=4.0 Hz, 1H), 7.31-7.34 (m, 1H), 7.40-7.43 (m, 1H), 7.47-7.59 (m, 5H). Chiral separation condition: MeOH contained 0.5% NH₄OH; IC (4.6×150 mm, 5 μm); retention time: 5.56 min.

### Example 105

### Synthesis of N-((2R)-3-(3-amino-8-azabicyclo[3.2.1]octan-8-yl)-1-(4-(cyclohexyloxy)phenyl)-1,1-difluoro-3-oxopropan-2-yl)-4-(cyclohexylmethoxy) benzenesulfonamide (Compound 105-1), and N-((2S)-3-(3-amino-8-azabicyclo[3.2.1]octan-8-yl)-1-(4-(cyclohexyloxy)phenyl)-1,1-difluoro-3-oxopropan-2-yl)-4-(cyclohexylmethoxy)benzenesulfonamide (Compound 105-2)

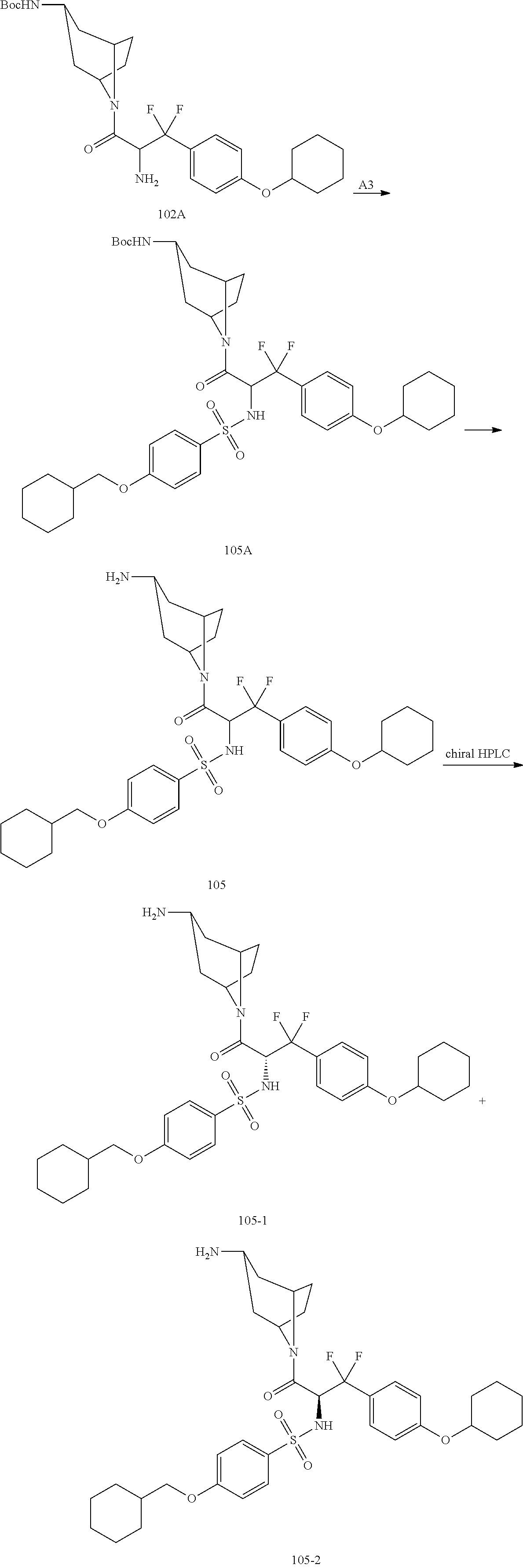

Compounds 105A and 105 were synthesized by employing the procedures described for Compounds 2D and 1 using Compound 102A, Compound A3, and Compound 105A in lieu of Compound B2, Compounds 2C, and 1A.

Compound 105A: LC-MS (ESI) m/z: 704 [M−55]⁺.

Compound 105 was separated with chiral HPLC to furnish Compound 105-1 and Compound 105-2.

Compound 105-1: LC-MS (ESI) m/z: 660 [M+H]⁺; ¹H-NMR (CD₃OD, 400 MHz): δ (ppm) 0.73-1.89 (m, 29H), 2.87-2.97 (m, 1H), 3.73-3.74 (m, 2H), 3.98-4.16 (m, 1H), 4.23-4.33 (m, 2H), 4.44-4.53 (m, 1H), 6.78-6.89 (m, 4H), 7.19-7.30 (m, 2H), 7.53-7.60 (m, 2H). Chiral separation condition: MeOH contained 0.5% NH₄OH; IC (250×4.6 mm, 5 μm); retention time: 4.77 minute.

Compound 105-2: LC-MS (ESI) m/z: 660 [M+H]⁺; ¹H-NMR (CD₃OD, 400 MHz): δ (ppm) 0.73-1.88 (m, 29H), 2.85-2.96 (m, 1H), 3.73-3.74 (m, 2H), 3.97-4.15 (m, 1H), 4.24-4.33 (m, 2H), 4.44-4.53 (m, 1H), 6.79-6.89 (m, 4H), 7.20-7.30 (m, 2H), 7.53-7.61 (m, 2H). Chiral separation condition: MeOH contained 0.5% NH₄OH; IC (250×4.6 mm, 5 μm); retention time: 3.97 minute.

### Example 106

### Synthesis of N-((2R)-3-(3-amino-8-azabicyclo[3.2.1]octan-8-yl)-1-(4-(benzyloxy) phenyl)-1,1-difluoro-3-oxopropan-2-yl)-6-(cyclopentyloxy)-N-methylnaphthalene-2-sulfonamide (Compound 106-1), and N-((2S)-3-(3-amino-8-azabicyclo[3.2.1]octan-8-yl)-1-(4-(benzyloxy)phenyl)-1,1-difluoro-3-oxopropan-2-yl)-6-(cyclopentyloxy)-N-methylnaphthalene-2-sulfonamide (Compound 106-2)

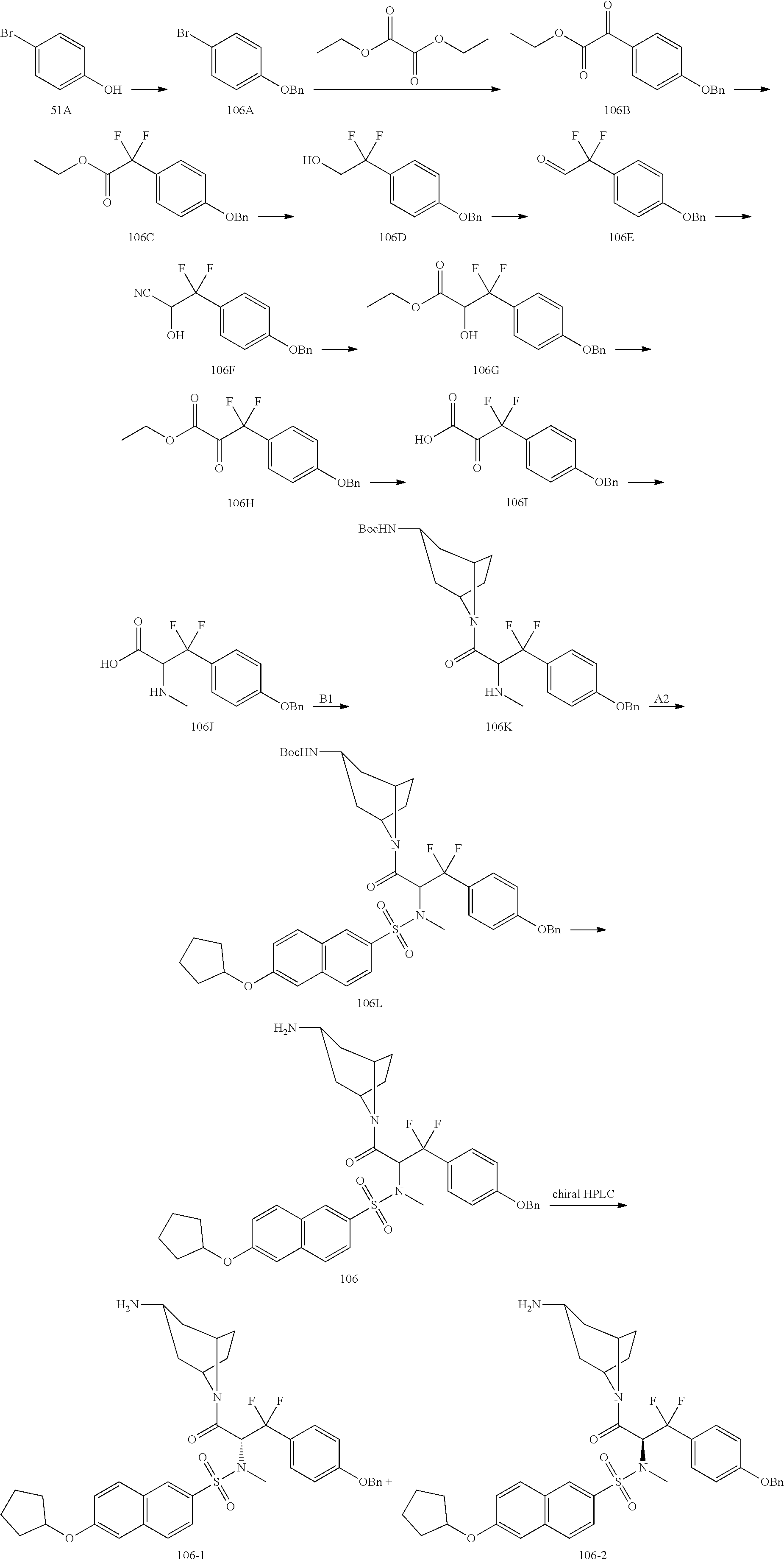

Compounds 106A, 106B, 106C, 106D, 106E, 106F, 106G, 106H, 106I, 106J, 106K, 106L, and 106 were synthesized by employing the procedures described for Compounds 4D, 9B, Compound B2-3, Compound 51E, Compound B6-3, Compounds 45F, 45G, Compound B6-3, Compounds 451, 80A, Compound B4, Compounds 1A, and 6 using (bromomethyl)benzene, Compounds 51A using K₂CO₃ as base at 50° C., 106A, 106B, 106C, 106D, 106E, 106F, 106G, 106H, 106I using MeNH2, 106J, Compound B1, Compound 106K, Compound A2, and Compound 106L in lieu of 2-iodopropane, Compounds 4C using CsCO₃ as base at room temperature, 9A, Compound B2-2, Compound 51D, Compound B6-2, Compounds 45E, 45F, Compound B6-2, Compounds 45H, 51J using NH₂OH hydrochloride, Compounds B2-6, B4-1, B2, A1, and Compound 6C.

Compound 106A: LC-MS (ESI) m/z: non-ionizable compound under routine conditions used; ¹H-NMR (CDCl₃, 400 MHz): δ (ppm) 5.03 (s, 2H), 6.84-6.86 (m, 2H), 7.31-7.42 (m, 7H).

Compound 106B was used for next step without further purification. LC-MS (ESI) m/z: 285 [M+H]⁺; ¹H-NMR (CDCl₃, 400 MHz): δ (ppm) 1.42 (t, J=7.2 Hz, 3H), 4.43 (q, J=7.2 Hz, 2H), 5.16 (s, 2H), 7.05 (d, J=8.8 Hz, 2H), 7.31-7.44 (m, 5H), 8.01 (d, J=8.8 Hz, 2H).

Compound 106: LC-MS (ESI) m/z: 287 [M−F]⁺; ¹H-NMR (CDCl₃, 400 MHz): δ (ppm) 1.30 (t, J=7.2 Hz, 3H), 4.29 (q, J=7.2 Hz, 2H), 5.09 (s, 2H), 7.02 (d, J=8.8 Hz, 2H), 7.32-7.43 (m, 5H), 7.53 (d, J=8.8 Hz, 2H).

Compound 106D: LC-MS (ESI) m/z: 245 [M−F]⁺; ¹H-NMR (CDCl₃, 400 MHz): δ (ppm) 1.96-1.99 (m, 1H), 3.91-3.99 (m, 2H), 5.09 (s, 2H), 7.02 (d, J=8.8 Hz, 2H), 7.32-7.45 (m, 7H).

Compound 106E was used directly for the next step.

Compound 106F: LC-MS (ESI) m/z: No; ¹H-NMR (CDCl₃, 400 MHz): δ (ppm) 3.30-3.36 (m, 1H), 4.67-4.71 (m, 1H), 5.03 (s, 2H), 6.99 (d, J=8.8 Hz, 2H), 7.25-7.43 (m, 7H).

Compound 106G: LC-MS (ESI) m/z: 317 [M−F]⁺; ¹H-NMR (CDCl₃, 400 MHz): δ (ppm) 1.26 (t, J=7.2 Hz, 3H), 3.23 (d, J=8.0 Hz, 1H), 4.23-4.29 (m, 2H), 4.47-4.54 (m, 1H), 5.09 (s, 2H), 7.01 (d, J=8.8 Hz, 2H), 7.32-7.44 (m, 7H).

Compound 106H: ¹H-NMR (CDCl₃, 400 MHz): δ (ppm) 1.35 (t, J=7.2 Hz, 3H), 4.35 (q, J=7.2 Hz, 2H), 5.07 (s, 2H), 6.99 (d, J=8.8 Hz, 2H), 7.31-7.43 (m, 5H), 7.51 (d, J=8.8 Hz, 2H).

Compound 1061: LC-MS (ESI) m/z: 305 [M−H]⁺; ¹H-NMR (DMSO-d₆, 400 MHz): δ (ppm) 5.14 (s, 2H), 7.04 (d, J=8.4 Hz, 2H), 7.32-7.47 (m, 7H), 13.25 (brs, 1H).

Compound 106J: LC-MS (ESI) m/z: 320 [M−H]⁺; ¹H-NMR (DMSO-d₆, 400 MHz): δ (ppm) 2.40 (s, 3H), 5.15 (s, 3H), 7.08 (d, J=8.8 Hz, 2H), 7.34-7.47 (m, 7H).

Compound 106K: LC-MS (ESI) m/z: 530 [M+H]⁺; ¹H-NMR (CDCl₃, 400 MHz): δ (ppm) 1.25-2.10 (m, 21H), 2.30-2.37 (m, 3H), 5.09 (s, 2H), 6.96-7.01 (m, 2H), 7.33-7.44 (m, 7H).

Compound 106L: LC-MS (ESI) m/z: 704 [M−Boc+H]⁺; ¹H-NMR: 1.11-1.50 (m, 12H), 1.66-2.06 (m, 13H), 3.10-3.26 (m, 3H), 3.88-3.97 (m, 1H), 4.08-4.64 (m, 2H), 4.89-4.93 (m, 1H), 5.06-5.09 (m, 2H), 5.36-5.49 (m, 1H), 6.91-7.00 (m, 2H), 7.13-7.23 (m, 2H), 7.33-7.47 (m, 7H), 7.60-7.67 (m, 1H), 7.74-7.88 (m, 2H), 8.23-8.24 (m, 1H).

Compound 106 was separated with chiral HPLC to give Compound 106-1 and Compound 106-2.

Compound 106-1: LC-MS (ESI) m/z: 704 [M+H]⁺; ¹H-NMR (CD₃OD, 400 MHz): δ (ppm) 1.13-2.05 (m, 16H), 3.03-3.09 (m, 1H), 3.15-3.30 (m, 3H), 4.19-4.58 (m, 2H), 4.94-5.05 (m, 3H), 5.39-5.53 (m, 1H), 6.92-7.03 (m, 2H), 7.22-7.47 (m, 9H), 7.53-7.63 (m, 1H), 7.83-7.94 (m, 2H), 8.21-8.23 (m, 1H). Chiral separation condition: MeOH contained 0.5% NH₄OH; (R,R)-whelk-O1 (4.6×250 mm, 5 μm); retention time: 6.95 minute.

Compound 106-2: LC-MS (ESI) m/z: 704 [M+H]⁺; ¹H-NMR (CD₃OD, 400 MHz): δ (ppm) 0.98-1.91 (m, 16H), 2.87-2.96 (m, 1H), 3.02-3.16 (m, 3H), 4.04-4.44 (m, 2H), 4.80-4.81 (m, 1H), 4.91 (s, 2H), 5.25-5.39 (m, 1H), 6.79-6.89 (m, 2H), 7.08-7.33 (m, 9H), 7.40-7.49 (m, 1H), 7.69-7.80 (m, 2H), 8.07-8.09 (m, 1H). Chiral separation condition: MeOH contained 0.5% NH₄OH; (R,R)-whelk-O1 (4.6×250 mm, 5 μm); retention time: 6.26 minute.

### Example 107

### Synthesis of N-((2R)-3-(3-amino-8-azabicyclo[3.2.1]octan-8-yl)-1,1-difluoro-3-oxo-1-(3-phenoxyphenyl)propan-2-yl)-6-(cyclopentyloxy)naphthalene-2-sulfonamide (Compound 107-1), and N-((2S)-3-(3-amino-8-azabicyclo[3.2.1]octan-8-yl)-1,1-difluoro-3-oxo-1-(3-phenoxyphenyl)propan-2-yl)-6-(cyclopentyloxy)naphthalene-2-sulfonamide (Compound 107-2)

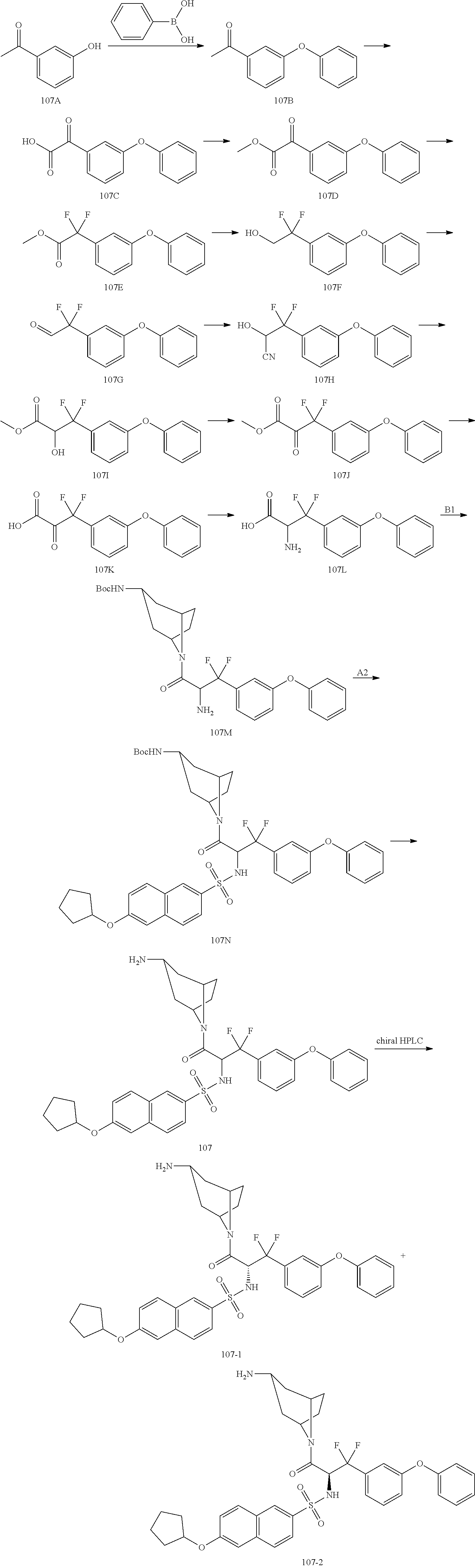

To a suspension of copper (II) acetate (5.32 g, 29.41 mmol), Compound 107A (8.00 g, 58.82 mmol), and powdered 4 Å molecular sieves (5.00 g) in 1,2-dichloroethane (100 mL) and pyridine (5 mL) at 20° C. was dropped a solution of phenylboronic acid (14.35 g, 117.65 mmol) in 1,2-dichloroethane (100 mL) and pyridine (5 mL) over 6 hours. The reaction mixture was stirred at 20° C. for 16 hours. More solution of phenylboronic acid (14.35 g, 117.65 mmol) in 1,2-dichloroethane (100 mL) and pyridine (5 mL) was dropped over 6 hours and stirred at 20° C. for another 16 hours. The reaction mixture was filtered and the filtrate was concentrated under reduced pressure. The residue was purified by silica gel column chromatography to furnish Compound 107B. ¹H-NMR (CDCl₃, 400 MHz): δ (ppm) 32.57 (s, 3H), 7.01 (d, J=8.0 Hz, 2H), 7.14-7.22 (m, 2H), 7.34-7.43 (m, 3H), 7.58 (s, 1H), 7.68 (d, J=7.2 Hz, 1H).

Compounds 107C, 107D, 107E, 107F, 107G, 107H, 107I, 107J, 107K, 107L, 107M, 107N, and 107 were synthesized by employing the procedures described for Compounds 42B, 89E, Compound B2-3, Compound 51E, Compound B6-3, Compounds 45F, 45G, Compound B6-3, Compounds 451, 80A, Compound B4, Compounds 2D, and 1 using Compounds 107B, 107C, 107D, 107E, 107F, 107G, 107H in MeOH, 1071, 107J, 107K, 107L, Compound B1, Compound 107M, Compound A2, and Compound 107N in lieu of Compounds 42A, 89D, Compound B2-2, Compound 51D, Compound B6-2, Compounds 45E, 45F in EtOH, Compound B6-2, Compounds 45H, 51J, Compounds B2-6, B4-1, B2, Compounds 2C, and 1A.

Compound 107C: LC-MS (ESI) m/z: 241 [M−H]⁺.

Compound 107D: ¹H-NMR (CDCl₃, 400 MHz): δ (ppm) 3.60 (s, 3H), 7.02 (d, J=8.0 Hz, 2H), 7.11-7.21 (m, 2H), 7.34-7.46 (m, 5H).

Compound 107E: LC-MS (ESI) m/z: 279 [M+H]⁺; ¹H-NMR (CDCl₃, 400 MHz): δ (ppm) 3.82 (s, 3H), 7.01 (d, J=8.0 Hz, 2H), 7.12-7.20 (m, 2H), 7.34-7.43 (m, 5H).

Compound 107F: LC-MS (ESI) m/z: 273 [M+Na]⁺; ¹H-NMR (CDCl₃, 400 MHz): δ (ppm) 2.16 (t, J=6.8 Hz, 1H), 3.89-3.99 (m, 2H), 7.00-7.05 (m, 3H), 7.07-7.12 (m, 2H), 7.16-7.25 (m, 1H), 7.33-7.41 (m, 3H).

Compound 107G was directly used for the next step without further purification. LC-MS (ESI) m/z: 249 [M+H]⁺.

Compound 107H: LC-MS (ESI) m/z: 298 [M+Na]⁺; ¹H-NMR (CDCl₃, 400 MHz): δ (ppm) 4.79 (t, J=8.8 Hz, 1H), 7.03 (d, J=8.0 Hz, 2H), 7.13-7.18 (m, 2H), 7.20-7.25 (m, 1H), 7.28-7.35 (m, 1H), 7.37-7.42 (m, 2H), 7.46-7.50 (m, 1H).

Compound 1071: LC-MS (ESI) m/z: 291 [M−F]⁺; ¹H-NMR (CDCl₃, 400 MHz): δ (ppm) 3.22 (d, J=8.0 Hz, 1H), 3.81 (s, 3H), 4.49-4.56 (m, 1H), 7.01 (d, J=8.0 Hz, 2H), 7.08-7.16 (m, 3H), 7.20-7.25 (m, 1H), 7.33-7.41 (m, 3H).

Compound 107J: LC-MS (ESI) m/z: 329 [M+Na]⁺; ¹H-NMR (CDCl₃, 400 MHz): δ (ppm) 3.92 (s, 3H), 7.02 (t, J=8.4 Hz, 2H), 7.08-7.16 (m, 2H), 7.21-7.24 (m, 1H), 7.30-7.41 (m, 4H).

Compound 107K: LC-MS (ESI) m/z: 291 [M−H]⁺; ¹H-NMR (CDCl₃, 400 MHz): δ (ppm) 7.00-7.03 (m, 2H), 7.04-7.15 (m, 2H), 7.20-7.28 (m, 1H), 7.32-7.39 (m, 4H).

Compound 107L: LC-MS (ESI) m/z: 294 [M+H]⁺; ¹H-NMR (CD₃OD, 400 MHz): δ (ppm) 4.83-4.94 (m, 1H), 7.00-7.21 (m, 5H), 7.33-7.42 (m, 3H), 7.49-7.54 (m, 1H).

Compound 107M: LC-MS (ESI) m/z: 502 [M+H]⁺.

Compound 107N: LC-MS (ESI) m/z: 798 [M+Na]⁺.

Compound 107 was separated with chiral HPLC to give Compound 107-1 and Compound 107-2.

Compound 107-1: LC-MS (ESI) m/z: 676 [M+H]⁺; ¹H-NMR (CD₃OD, 400 MHz): δ (ppm) 1.12-1.95 (m, 16H), 3.21-3.58 (m, 1H), 4.21-4.77 (m, 3H), 4.86-4.93 (m, 1H), 6.64-7.29 (m, 11H), 7.46-7.54 (m, 1H), 7.66-7.77 (m, 2H), 8.04-8.09 (m, 1H). Chiral separation condition: MeOH contained 0.2% Methanol Ammonia; IC (4.6×150 mm, 5 μm); retention time: 7.46 minute.

Compound 107-2: LC-MS (ESI) m/z: 676 [M+H]⁺; ¹H-NMR (CD₃OD, 400 MHz): δ (ppm) 0.36-1.15 (m, 3H), 1.19-2.04 (m, 13H), 2.74-3.00 (m, 1H), 4.12-4.34 (m, 2H), 4.60-4.70 (m, 1H), 4.95-5.01 (m, 1H), 6.98-7.40 (m, 11H), 7.64-7.92 (m, 3H), 8.21-8.29 (m, 1H). Chiral separation condition: MeOH contained 0.2% Methanol Ammonia; IC (4.6×150 mm, 5 μm); retention time: 5.66 minute.

### Example 108

### Synthesis of (R)—N-(3-(4-aminopiperidin-1-yl)-1,1-difluoro-3-oxo-1-(3-phenoxyphenyl)propan-2-yl)-6-(cyclopentyloxy)naphthalene-2-sulfonamide (Compound 108-1), and (S)—N-(3-(4-aminopiperidin-1-yl)-1,1-difluoro-3-oxo-1-(3-phenoxyphenyl)propan-2-yl)-6-(cyclopentyloxy)naphthalene-2-sulfonamide (Compound 108-2)

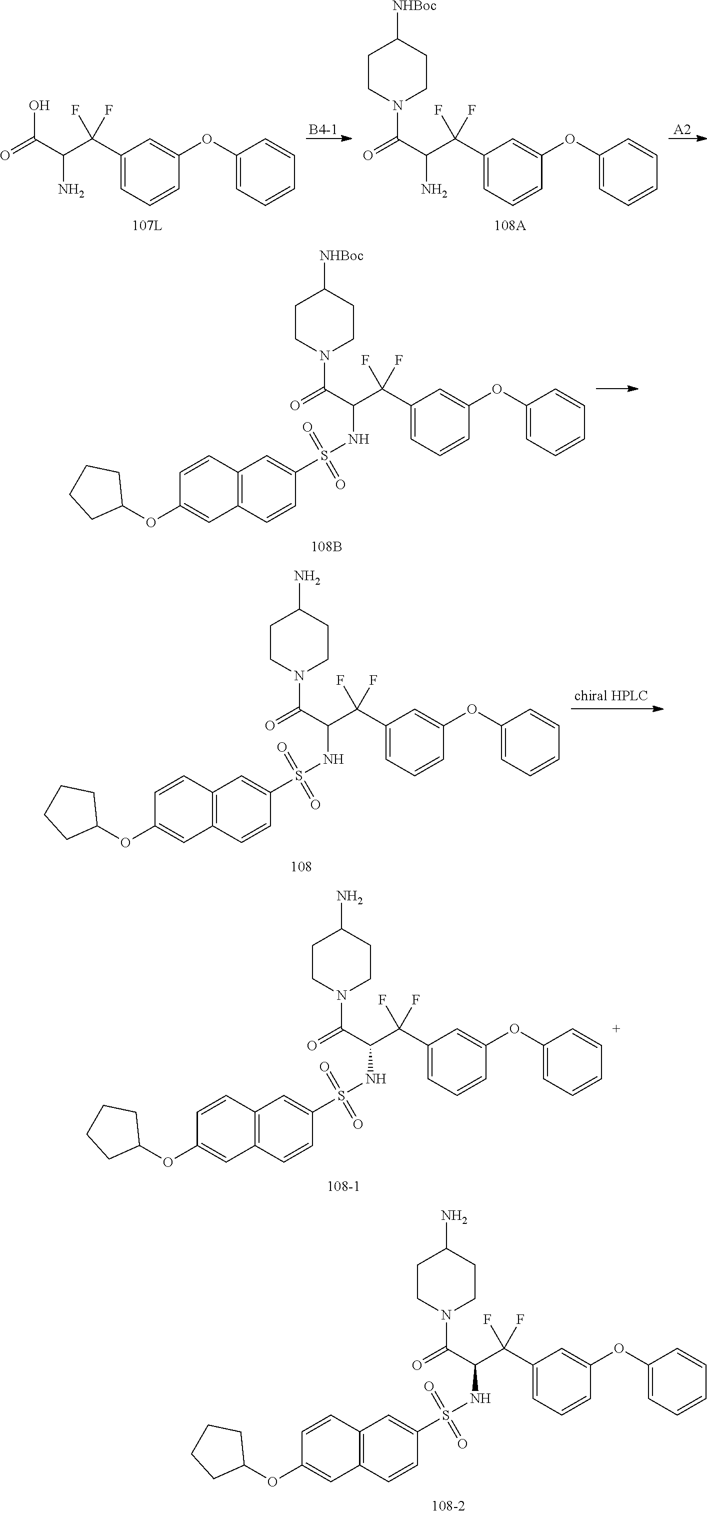

Compounds 108A, 108B, and 108 were synthesized by employing the procedures described for Compound B4, Compounds 2D, and 6 using Compounds 107L, 108A, Compound A2, and Compound 108B in lieu of Compounds B2-6, B2, Compounds 2C, and 6C.

Compound 108A: LC-MS (ESI) m/z: 476 [M+H]⁺.

Compound 108B: LC-MS (ESI) m/z: 750 [M+H]⁺.

Compound 108 was separated with chiral HPLC to furnish Compound 108-1 and Compound 108-2.

Compound 108-1: LC-MS (ESI) m/z: 650 [M+H]⁺; ¹H-NMR (CD₃OD, 400 MHz): δ (ppm) 1.34-2.08 (m, 12H), 2.72-2.92 (m, 1H), 3.11-3.17 (m, 1H), 3.31-3.41 (m, 1H), 4.08-4.52 (m, 2H), 4.92-5.11 (m, 2H), 6.77-7.39 (m, 11H), 7.51-7.88 (m, 3H), 8.13-8.17 (m, 1H). Chiral separation condition: n-hexane/EtOH contained 0.1% DEA, 70/30; OZ-H (4.6×250 mm, 5 μm); retention time: 17.44 minute.

Compound 108-2: LC-MS (ESI) m/z: 650 [M+H]⁺; ¹H-NMR (CD₃OD, 400 MHz): δ (ppm) 1.18-2.06 (m, 12H), 2.69-2.90 (m, 1H), 3.11-3.17 (m, 1H), 3.31-3.41 (m, 1H), 4.08-4.52 (m, 2H), 4.92-5.11 (m, 2H), 6.77-7.39 (m, 11H), 7.51-7.88 (m, 3H), 8.13-8.17 (m, 1H). Chiral separation condition: n-hexane/EtOH contained 0.1% DEA, 70/30; OZ-H (4.6×250 mm, 5 μm); retention time: 12.03 minute.

### Example 109

### Synthesis of (R)—N-(3-(4-aminopiperidin-1-yl)-1,1-difluoro-3-oxo-1-(3-phenoxyphenyl)propan-2-yl)-4-(cyclohexylmethoxy)benzenesulfonamide trifluoroacetate (Compound 109-1), and (S)—N-(3-(4-aminopiperidin-1-yl)-1,1-difluoro-3-oxo-1-(3-phenoxyphenyl)propan-2-yl)-4-(cyclohexylmethoxy) benzenesulfonamide trifluoroacetate (Compound 109-2)

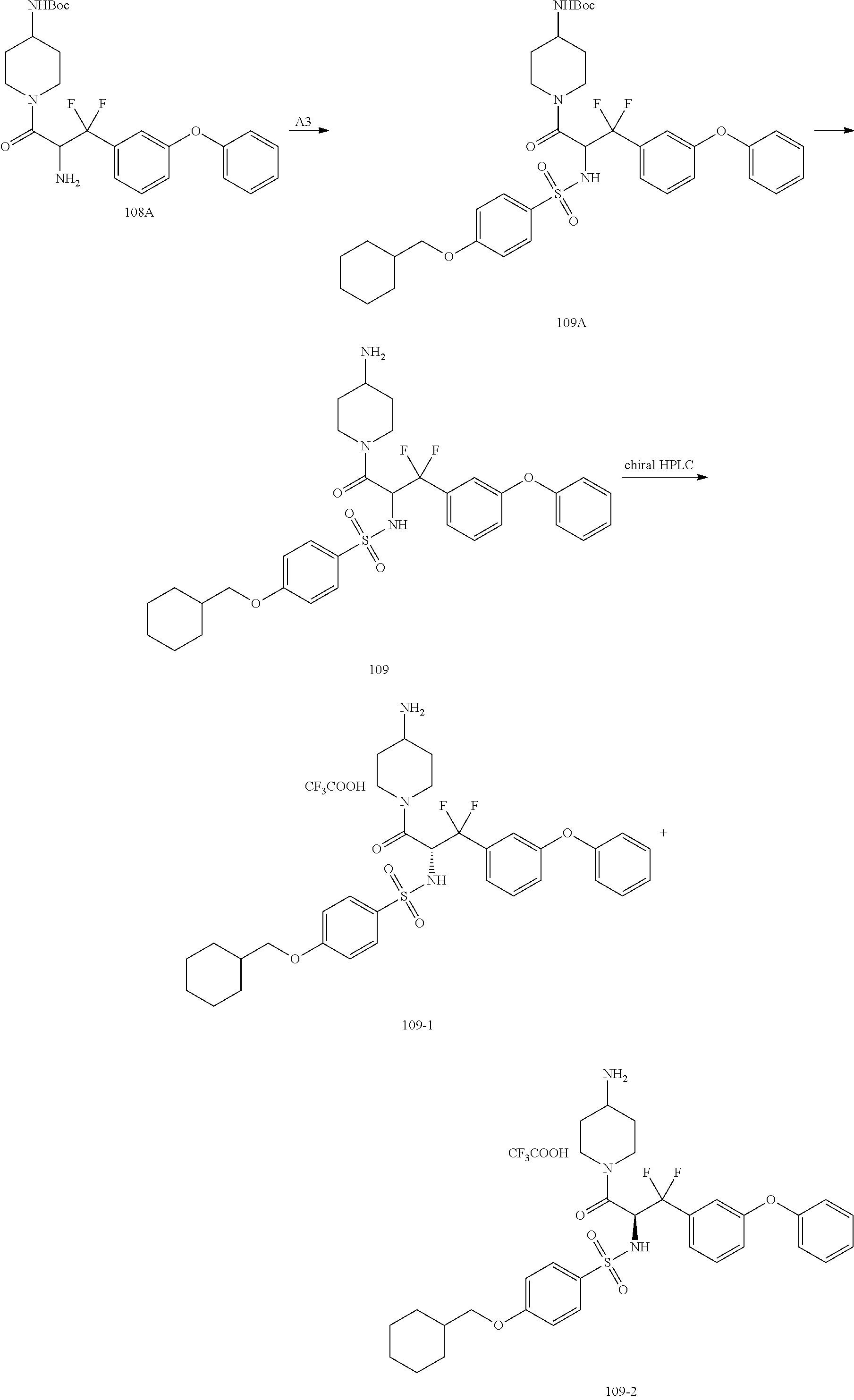

Compounds 109A and 109 were synthesized by employing the procedures described for Compounds 2D and 6 using Compound 108A, Compound A3, and Compound 109A in lieu of Compound B2, Compounds 2C, and 6C.

Compound 109A: LC-MS (ESI) m/z: 729 [M+H]⁺.

Compound 109 was separated with chiral HPLC to furnish Compound 109-1 and Compound 109-2.

Compound 109-1: LC-MS (ESI) m/z: 628 [M+H]⁺; ¹H-NMR (CD₃OD, 400 MHz): δ (ppm) 1.08-2.10 (m, 15H), 2.36-2.75 (m, 1H), 3.02-3.15 (m, 1H), 3.31-3.39 (m, 1H), 3.83-3.85 (m, 2H), 4.13-4.52 (m, 2H), 4.99-5.06 (m, 1H), 6.92-7.17 (m, 8H), 7.37-7.43 (m, 3H), 7.55-7.62 (m, 2H). Chiral separation condition: MeOH contained 0.5% NH₄OH; IC (4.6×150 mm, 5 μm); retention time: 6.47 minute.

Compound 109-2: LC-MS (ESI) m/z: 628 [M+H]⁺; ¹H-NMR (CD₃OD, 400 MHz): δ (ppm) 1.08-2.09 (m, 15H), 2.37-2.75 (m, 1H), 3.02-3.15 (m, 1H), 3.31-3.39 (m, 1H), 3.83-3.85 (m, 2H), 4.13-4.52 (m, 2H), 4.99-5.06 (m, 1H), 6.92-7.17 (m, 8H), 7.37-7.43 (m, 3H), 7.55-7.61 (m, 2H). Chiral separation condition: MeOH contained 0.5% NH₄OH; IC (4.6×150 mm, 5 μm); retention time: 5.04 minute.

### Example 110

### Synthesis of N-((2R)-3-(3-amino-8-azabicyclo[3.2.1]octan-8-yl)-1,1-difluoro-1-(4-hydroxyphenyl)-3-oxopropan-2-yl)-6-(cyclopentyloxy)-N-methylnaphthalene-2-sulfonamide trifluoroacetate (Compound 110-1), and N-((2S)-3-(3-amino-8-azabicyclo[3.2.1]octan-8-yl)-1,1-difluoro-1-(4-hydroxyphenyl)-3-oxopropan-2-yl)-6-(cyclopentyloxy)-N-methylnaphthalene-2-sulfonamide trifluoroacetate (Compound 110-2)

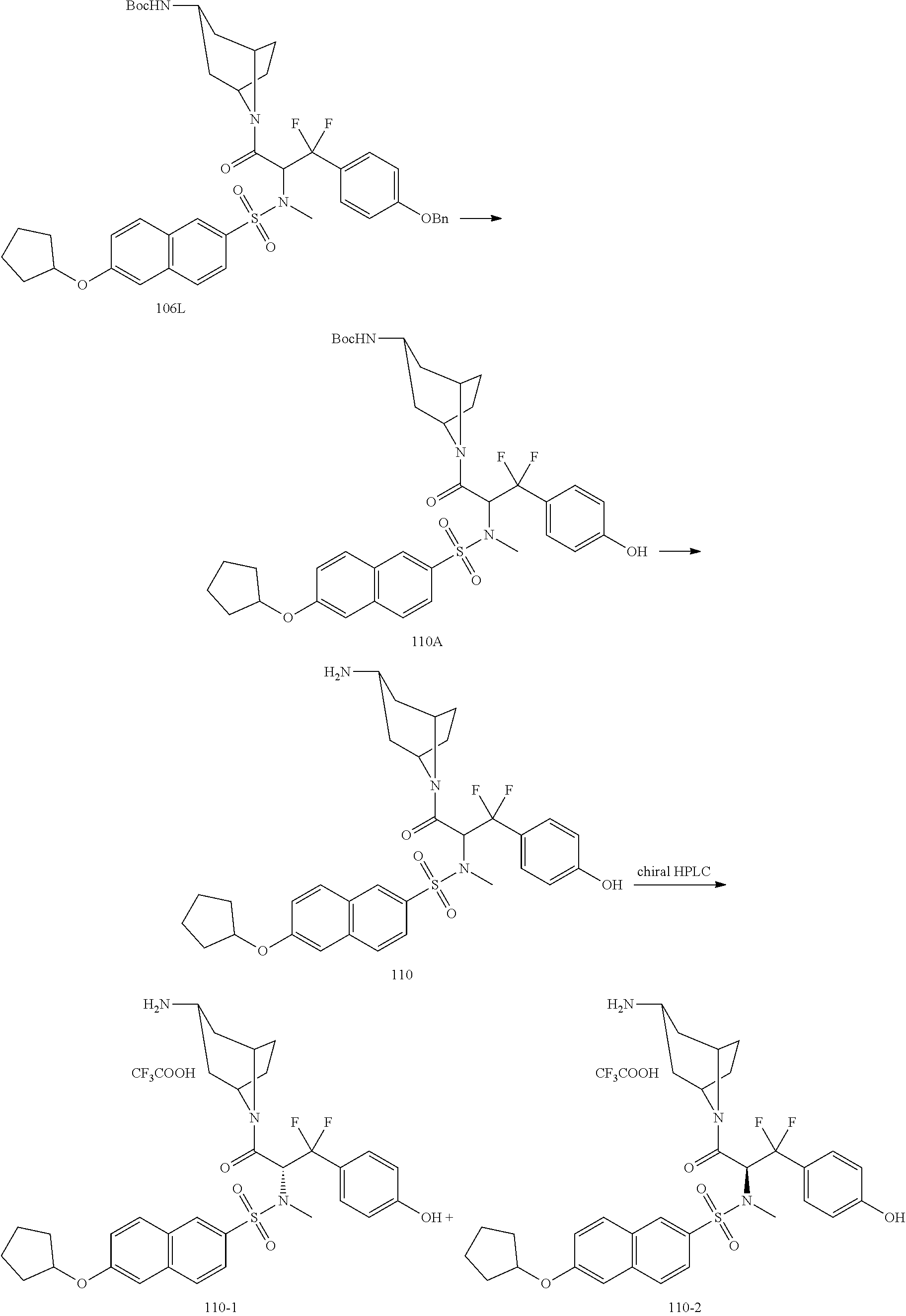

A mixture of Compound 106L (440 mg, 0.55 mmol) and 10% Pd/C (500 mg) in methanol (40 mL) was stirred under hydrogen at 35° C. for 16 hours. The reaction mixture was filtered and washed with dichloromethane (50 mL). The filtrate was concentrated to give Compound 110A. LC-MS (ESI) m/z: 614 [M−Boc+H]⁺.

Compound 110 was synthesized by employing the procedure described for Compound 6 using Compound 110A in lieu of Compound 6C, and was separated with chiral HPLC to give Compound 110-1 and Compound 110-2.

Compound 110-1: LC-MS (ESI) m/z: 614 [M+H]⁺; ¹H-NMR (CD₃OD, 400 MHz): δ (ppm) 1.42-2.21 (m, 16H), 3.01-3.24 (m, 3H), 3.60-3.72 (m, 1H), 4.41-4.44 (m, 1H), 4.64-4.74 (m, 1H), 5.00-5.05 (m, 1H), 5.49-5.58 (m, 1H), 6.76-6.78 (m, 2H), 7.23-7.36 (m, 4H), 7.61-7.64 (m, 1H), 7.78-7.96 (m, 2H), 8.05-8.27 (m, 1H). Chiral separation condition: n-hexane/MeOH contained 0.1% DEA, 70/30; AY-H (4.6×250 mm, 5 μm); retention time: 10.91 minute.

Compound 110-2: LC-MS (ESI) m/z: 614 [M+H]⁺; ¹H-NMR (CD₃OD, 400 MHz): δ (ppm) 1.31-2.18 (m, 16H), 3.00-3.23 (m, 3H), 3.59-3.70 (m, 1H), 4.41-4.42 (m, 1H), 4.63-4.72 (m, 1H), 4.99-5.04 (m, 1H), 5.48-5.57 (m, 1H), 6.75-6.77 (m, 2H), 7.22-7.35 (m, 4H), 7.61-7.63 (m, 1H), 7.77-7.95 (m, 2H), 8.04-8.26 (m, 1H). Chiral separation condition: n-hexane/MeOH contained 0.1% DEA, 70/30; AY-H (4.6×250 mm, 5 μm); retention time: 8.30 minute.

### Example 111

### Synthesis of N-((2R)-3-(3-amino-8-azabicyclo[3.2.1]octan-8-yl)-1,1-difluoro-3-oxo-1-(3-phenoxyphenyl)propan-2-yl)-6-(cyclopentyloxy)-N-methylnaphthalene-2-sulfonamide trifluoroacetate (Compound 111-1), and N-((2S)-3-(3-amino-8-azabicyclo[3.2.1]octan-8-yl)-1,1-difluoro-3-oxo-1-(3-phenoxyphenyl)propan-2-yl)-6-(cyclopentyloxy)-N-methylnaphthalene-2-sulfonamide trifluoroacetate (Compound 111-2)

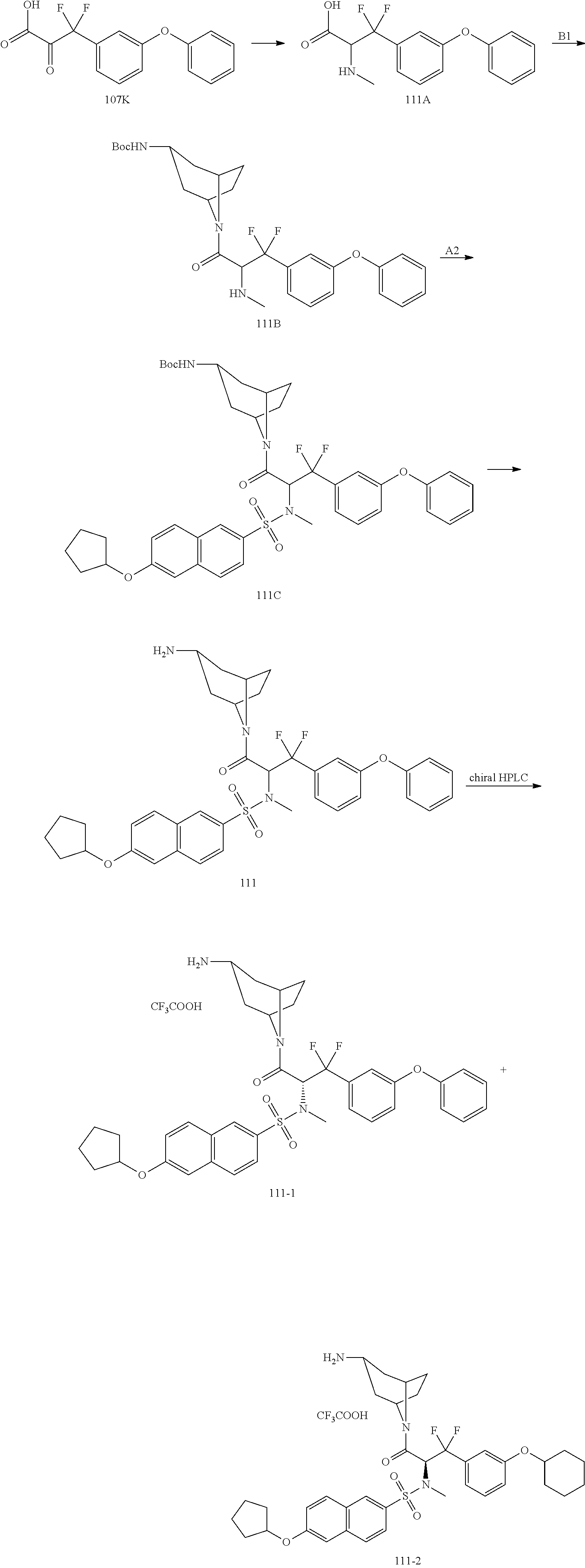

Compounds 111A, 111B, 111C, and 111 were synthesized by employing the procedures described for Compound 80A, Compound B4, Compounds 2D, and 1 using Compounds 107K with methylamine, 111A, Compound B1, Compound 111B, Compound A2, and Compound 111C in lieu of Compound 51J with hydroxylamine hydrochloride, Compounds B2-6, B4-1, B2, Compounds 2C, and 1A.

Compound 111A: LC-MS (ESI) m/z: 308 [M+H]⁺.

Compound 111B: LC-MS (ESI) m/z: 516 [M+H]⁺.

Compound 111C: LC-MS (ESI) m/z: 812 [M+Na]⁺.

Compound 111 was separated with chiral HPLC to give Compound 111-1 and Compound 111-2.

Compound 111-1: LC-MS (ESI) m/z: 690 [M+H]⁺; ¹H-NMR (CD₃OD, 400 MHz): δ (ppm) 1.45-2.11 (m, 16H), 2.87 (s, 1H), 3.08 (s, 2H), 3.51-3.63 (m, 1H), 4.40-4.78 (m, 2H), 4.86-4.94 (m, 1H), 5.40-5.53 (m, 1H), 6.77 (d, J=8.0 Hz, 2H), 6.87-7.47 (m, 10H), 7.69-7.82 (m, 2H), 8.00-8.14 (m, 1H). Chiral separation condition: MeOH contained 0.5% Methanol Ammonia; AD-H (4.6×150 mm, 5 μm); retention time: 7.41 minute.

Compound 111-2: LC-MS (ESI) m/z: 690 [M+H]⁺; ¹H-NMR (CD₃OD, 400 MHz): δ (ppm) 1.58-2.21 (m, 16H), 2.98 (s, 1H), 3.19 (s, 2H), 3.64-3.76 (m, 1H), 4.51-4.72 (m, 2H), 4.98-5.03 (m, 1H), 5.53-5.63 (m, 1H), 6.88 (d, J=8.0 Hz, 2H), 6.96-7.58 (m, 10H), 7.77-7.92 (m, 2H), 8.11-8.25 (m, 1H). Chiral separation condition: MeOH contained 0.5% Methanol Ammonia; AD-H (4.6×150 mm, 5 μm); retention time: 4.07 minute.

### Example 112

### Synthesis of (R)—N-(3-(4-aminopiperidin-1-yl)-1-(4-(5-chlorothiophen-2-yl)phenyl)-1,1-difluoro-3-oxopropan-2-yl)-6-(cyclopentyloxy)naphthalene-2-sulfonamide trifluoroacetate (Compound 112-1), and (S)—N-(3-(4-aminopiperidin-1-yl)-1-(4-(5-chlorothiophen-2-yl)phenyl)-1,1-difluoro-3-oxopropan-2-yl)-6-(cyclopentyloxy)naphthalene-2-sulfonamide trifluoroacetate (Compound 112-2)

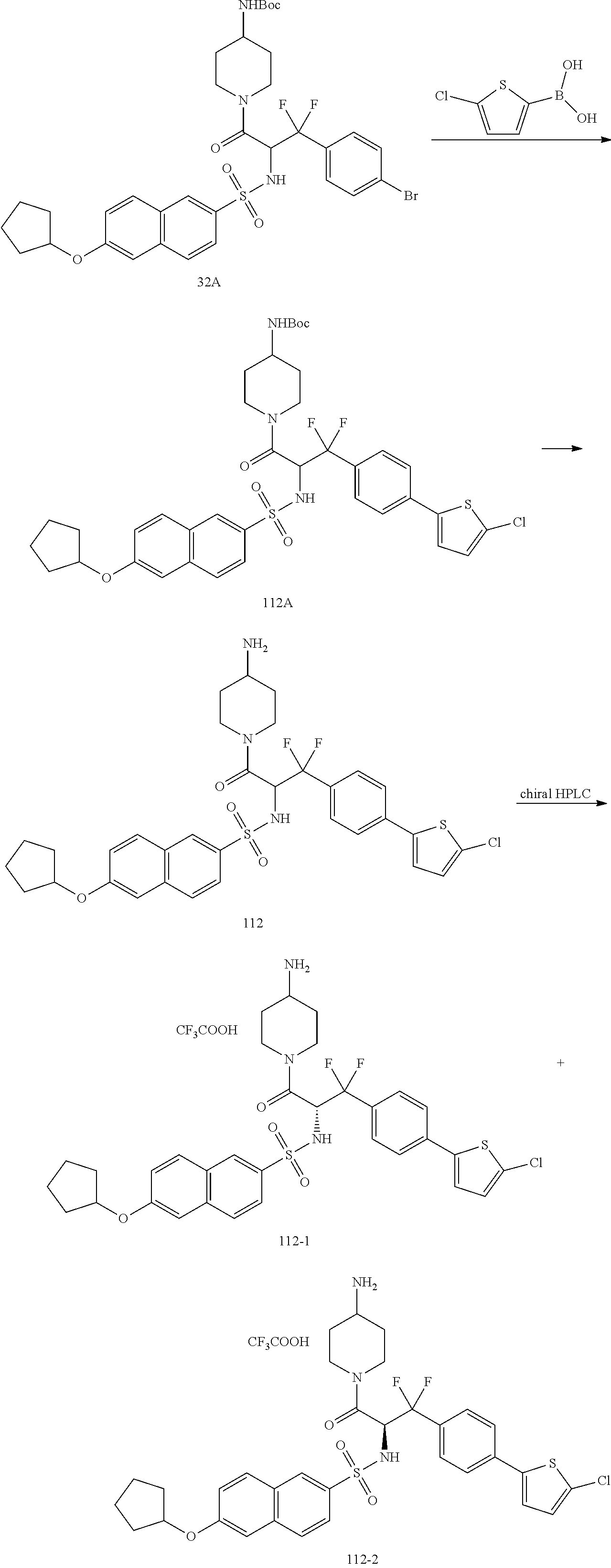

Compounds 112A and 112 were synthesized by employing the procedures described for Compounds 14C and 6 using 5-chlorothiophen-2-ylboronic acid, Compounds 32A and 112A in lieu of Compounds 14B, 14A, and 6C.

Compound 112A: LC-MS (ESI) m/z: 774 [M+H]⁺.

Compound 112 was separated with chiral HPLC to furnish Compound 112-1 and Compound 112-2.

Compound 112-1: LC-MS (ESI) m/z: 674 [M+H]⁺; ¹H-NMR (CD₃OD, 400 MHz): δ (ppm) 1.40-2.06 (m, 12H), 2.18-2.87 (m, 1H), 3.09-3.26 (m, 1H), 3.31-3.47 (m, 1H), 4.26-4.86 (m, 2H), 4.97-5.09 (m, 2H), 697-7.41 (m, 9H), 7.50-7.76 (m, 2H), 7.95-8.05 (m, 1H). Chiral separation condition: MeOH contained 0.2% methanol ammonia; IC (4.6×150 mm, 5 μm); retention time: 9.65 minute.

Compound 112-2: LC-MS (ESI) m/z: 674 [M+H]⁺; ¹H-NMR (CD₃OD, 400 MHz): δ (ppm) 1.33-2.21 (m, 12H), 2.28-2.87 (m, 1H), 3.08-3.26 (m, 1H), 3.31-3.49 (m, 1H), 4.26-4.86 (m, 2H), 4.97-5.09 (m, 2H), 6.97-7.41 (m, 9H), 7.50-7.76 (m, 2H), 7.96-8.06 (m, 1H). Chiral separation condition: MeOH contained 0.2% methanol ammonia; IC (4.6×150 mm, 5 μm); retention time: 7.78 minute.

### Example 113

### Synthesis of N-((2R)-3-(3-amino-8-azabicyclo[3.2.1]octan-8-yl)-1,1-difluoro-3-oxo-1-(3-phenoxyphenyl)propan-2-yl)-4-(cyclohexylmethoxy)benzenesulfonamide trifluoroacetate (Compound 113-1), and N-((2S)-3-(3-amino-8-azabicyclo[3.2.1]octan-8-yl)-1,1-difluoro-3-oxo-1-(3-phenoxyphenyl)propan-2-yl)-4-(cyclohexylmethoxy) benzenesulfonamide trifluoroacetate (Compound 113-2)

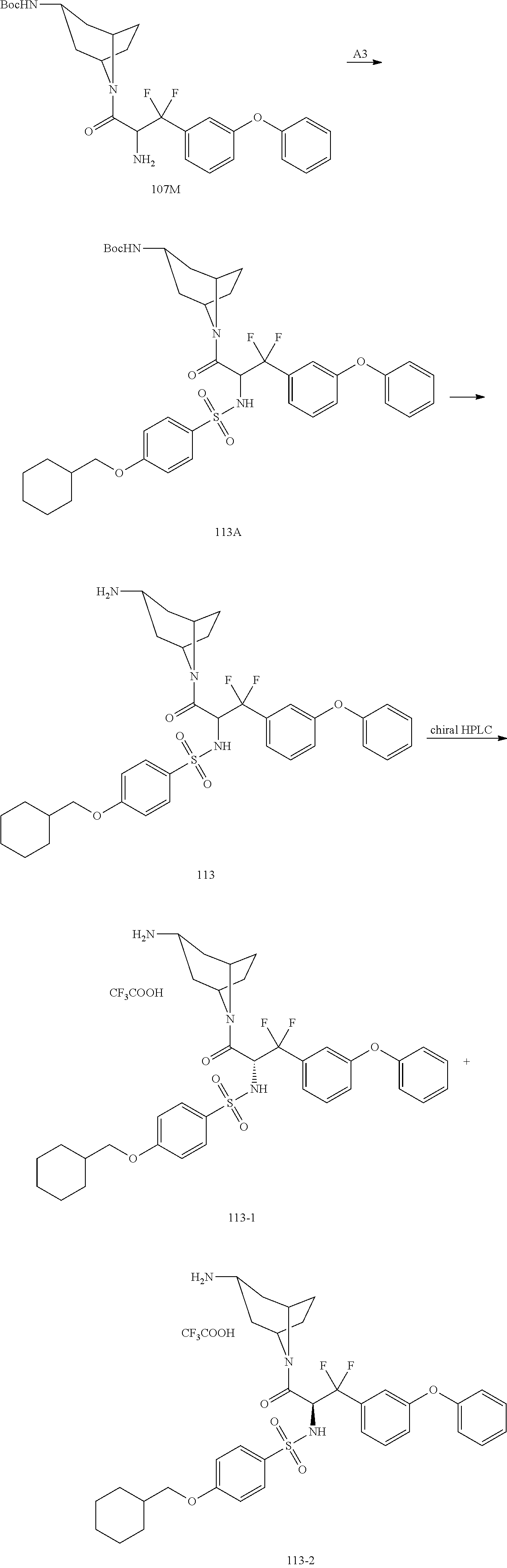

Compounds 113A and 113 were synthesized by employing the procedures described for Compounds 2D and 1 using Compound 107M, Compound A3, and Compound 113A in lieu of Compound B2, Compounds 2C, and 1A.

Compound 113A: LC-MS (ESI) m/z: 776 [M+Na]⁺.

Compound 113 was separated with chiral HPLC to afford Compound 113-1 and Compound 113-2.

Compound 113-1: LC-MS (ESI) m/z: 654 [M+H]⁺; ¹H-NMR (CD₃OD, 400 MHz): δ (ppm) 1.06-2.10 (m, 19H), 3.58-3.74 (m, 1H), 3.82-3.86 (m, 2H), 4.52-4.86 (m, 3H), 6.92-7.46 (m, 11H), 7.61 (t, J=8.8 Hz, 2H). Chiral separation condition: n-Hexane/EtOH contained 0.1% DEA, 80/20; OJ-H (4.6×250 mm, 5 μm); retention time: 15.28 minute.

Compound 113-2: LC-MS (ESI) m/z: 654 [M+H]⁺; ¹H-NMR (CD₃OD, 400 MHz): δ (ppm) 1.03-2.14 (m, 19H), 3.56-3.73 (m, 1H), 3.82-3.86 (m, 2H), 4.52-4.85 (m, 3H), 6.92-7.44 (m, 11H), 7.61 (t, J=8.8 Hz, 2H). Chiral separation condition: n-Hexane/EtOH contained 0.1% DEA, 80/20; OJ-H (4.6×250 mm, 5 μm); retention time: 9.97 minute.

### Example 114

### Synthesis of N-((2R)-3-(3-amino-8-azabicyclo[3.2.1]octan-8-yl)-1-(4-ethoxyphenyl)-1,1-difluoro-3-oxopropan-2-yl)-6-(cyclopentyloxy)-N-methylnaphthalene-2-sulfonamide (Compound 114-1), and N-((2S)-3-(3-amino-8-azabicyclo[3.2.1]octan-8-yl)-1-(4-ethoxyphenyl)-1,1-difluoro-3-oxopropan-2-yl)-6-(cyclopentyloxy)-N-methylnaphthalene-2-sulfonamide (Compound 114-2)

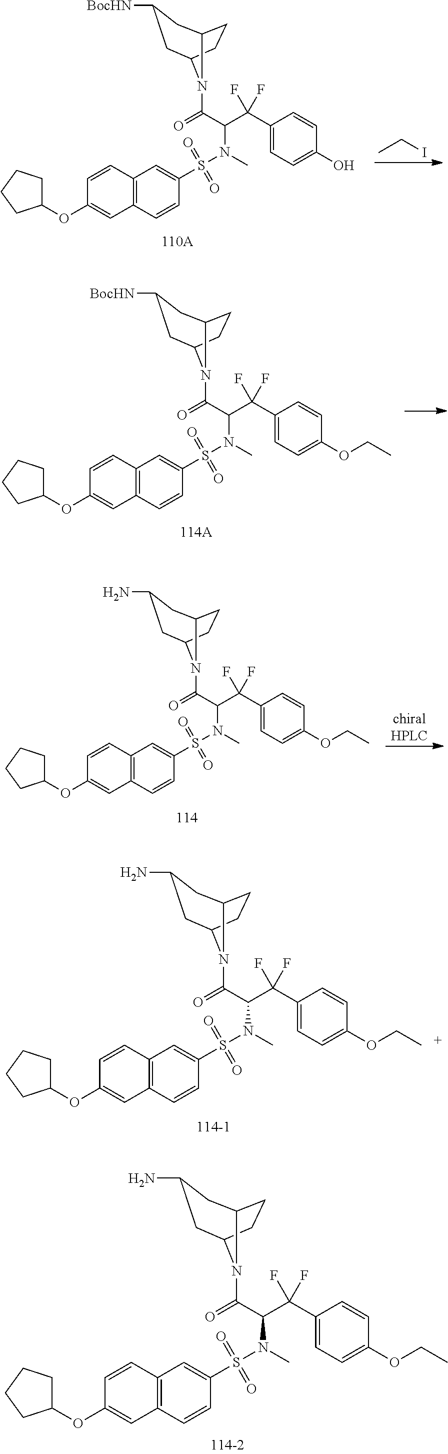

Compounds 114A and 114 were synthesized by employing the procedures described for Compounds 4D and 6 using Compounds 110A with iodoethane using K₂CO₃ as base at 50° C. and 114A in lieu of Compounds 4C with 2-iodopropane using Cs₂CO₃ as base at 25° C., and 6C.

Compound 114A: LC-MS (ESI) m/z: 642 [M−Boc+1].

Compound 114 was separated with chiral HPLC to afford Compound 114-1 and Compound 114-2.

Compound 114-1: LC-MS (ESI) m/z: 642 [M+H]; ¹H-NMR (CD₃OD, 400 MHz): δ (ppm) 1.19-1.29 (m, 5H), 1.41-1.99 (m, 16H), 3.04-3.19 (m, 3H), 3.84-3.92 (m, 2H), 4.42-4.46 (m, 1H), 4.89-4.92 (m, 1H), 5.27-5.49 (m, 1H), 6.73-6.83 (m, 2H), 7.11-7.15 (m, 1H), 7.20-7.23 (m, 2H), 7.31-7.33 (m, 1H), 7.46-7.56 (m, 1H), 7.74-7.82 (m, 2H), 8.09-8.12 (m, 1H). Chiral separation condition: n-Hexane/EtOH contained 0.1% DEA, 60/40; AY-H (4.6×250 mm, 5 μm); retention time: 23.58 minute.

Compound 114-2: LC-MS (ESI) m/z: 642 [M+H]⁺; ¹H-NMR (CD₃OD, 400 MHz): δ (ppm) 1.19-1.31 (m, 5H), 1.42-2.00 (m, 16H), 3.04-3.19 (m, 3H), 3.84-3.93 (m, 2H), 4.43-4.47 (m, 1H), 4.89-4.92 (m, 1H), 5.27-5.44 (m, 1H), 6.73-6.83 (m, 2H), 7.12-7.15 (m, 1H), 7.20-7.23 (m, 2H), 7.31-7.33 (m, 1H), 7.45-7.54 (m, 1H), 7.74-7.82 (m, 2H), 8.09-8.12 (m, 1H). Chiral separation condition: n-Hexane/EtOH contained 0.1% DEA, 60/40; AY-H (4.6×250 mm, 5 μm); retention time: 9.05 minute.

### Example 115

### Synthesis of N-((2R)-3-(3-amino-8-azabicyclo[3.2.1]octan-8-yl)-1,1-difluoro-1-(4-(2-morpholinoethoxy)phenyl)-3-oxopropan-2-yl)-6-(cyclopentyloxy)-N-methylnaphthalene-2-sulfonamide (Compound 115-1), and N-((2S)-3-(3-amino-8-azabicyclo[3.2.1]octan-8-yl)-1,1-difluoro-1-(4-(2-morpholinoethoxy)phenyl)-3-oxopropan-2-yl)-6-(cyclopentyloxy)-N-methylnaphthalene-2-sulfonamide (Compound 115-2)

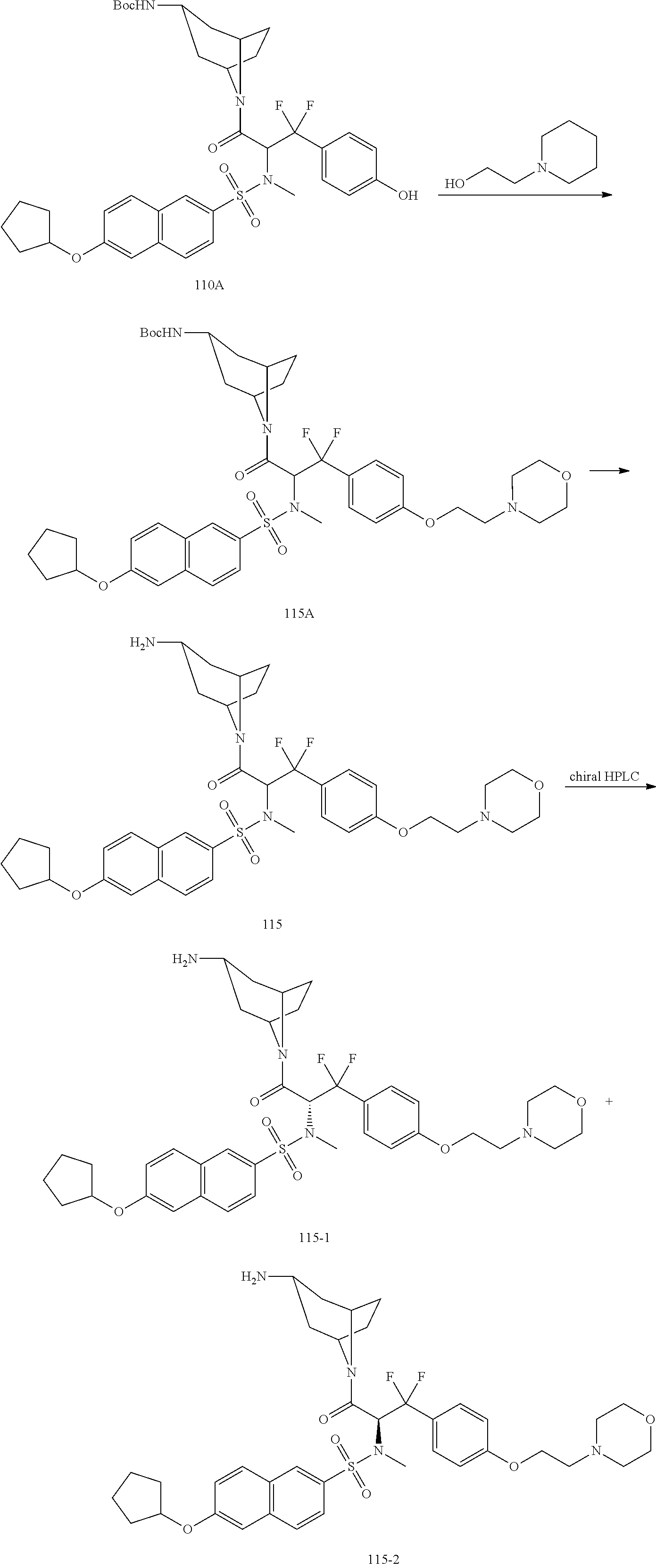

A mixture of Compound 110A (200 mg, 0.28 mmol), 2-morpholinoethanol (55 mg, 0.42 mmol), PPh₃ (147 mg, 0.56 mmol), and DEAD (170 mg, 0.84 mmol) in toluene (4 mL) and dichloromethane (1 mL) was stirred at room temperature for 2 hours. The reaction mixture was diluted with water (50 mL) and extracted with ethyl acetate (100 mL×2). The combined organic layers was washed with water (100 mL×2) and brine (100 mL), dried over sodium sulfate, filtered, and concentrated under reduced pressure. The residue was purified with reverse phase chromatography using eluent (methanol in water, from 0% to 100% v/v) to give Compound 115A. LC-MS (ESI) m/z: non-ionizable compound under routine conditions used.

Compound 115 was synthesized by employing the procedure described for Compound 6 using Compound 115A in lieu of Compound 6C, and was separated with chiral HPLC to afford Compound 115-1 and Compound 115-2.

Compound 115-1: LC-MS (ESI) m/z: 727 [M+H]⁺; ¹H-NMR (CD₃OD, 400 MHz): δ (ppm) 1.24-1.98 (m, 18H), 2.47-2.49 (m, 4H), 2.68-2.72 (m, 2H), 3.02-3.17 (m, 3H), 3.60-3.62 (m, 4H), 3.98-4.03 (m, 2H), 4.43-4.46 (m, 1H), 4.90-4.93 (m, 1H), 5.30-5.50 (m, 1H), 6.79-6.88 (m, 2H), 7.13-7.16 (m, 1H), 7.21-7.35 (m, 3H), 7.43-7.56 (m, 1H), 7.74-7.85 (m, 2H), 8.09-8.14 (m, 1H). Chiral separation condition: n-Hexane/EtOH contained 0.1% DEA, 50/50; AY-H (4.6×250 mm, 5 μm); retention time: 22.13 minute.

Compound 115-2: LC-MS (ESI) m/z: 727 [M+H]⁺; ¹H-NMR (CD₃OD, 400 MHz): δ (ppm) 1.25-2.00 (m, 18H), 2.47-2.49 (m, 4H), 2.68-2.71 (m, 2H), 3.02-3.17 (m, 3H), 3.60-3.62 (m, 4H), 3.97-4.02 (m, 2H), 4.44-4.46 (m, 1H), 4.89-4.92 (m, 1H), 5.30-5.45 (m, 1H), 6.79-6.88 (m, 2H), 7.12-7.16 (m, 1H), 7.21-7.35 (m, 3H), 7.44-7.56 (m, 1H), 7.74-7.82 (m, 2H), 8.09-8.14 (m, 1H). Chiral separation condition: n-Hexane/EtOH contained 0.1% DEA, 50/50; AY-H (4.6×250 mm, 5 μm); retention time: 10.94 minute.

### Example 116

### Synthesis of N-((2R)-3-(3-amino-8-azabicyclo[3.2.1]octan-8-yl)-1,1-difluoro-3-oxo-1-(4-(2-(piperidin-1-yl)ethoxy)phenyl)propan-2-yl)-6-(cyclopentyloxy)-N-methylnaphthalene-2-sulfonamide (Compound 116-1), and N-((2S)-3-(3-amino-8-azabicyclo[3.2.1]octan-8-yl)-1,1-difluoro-3-oxo-1-(4-(2-(piperidin-1-yl)ethoxy)phenyl)propan-2-yl)-6-(cyclopentyloxy)-N-methylnaphthalene-2-sulfonamide (Compound 116-2)

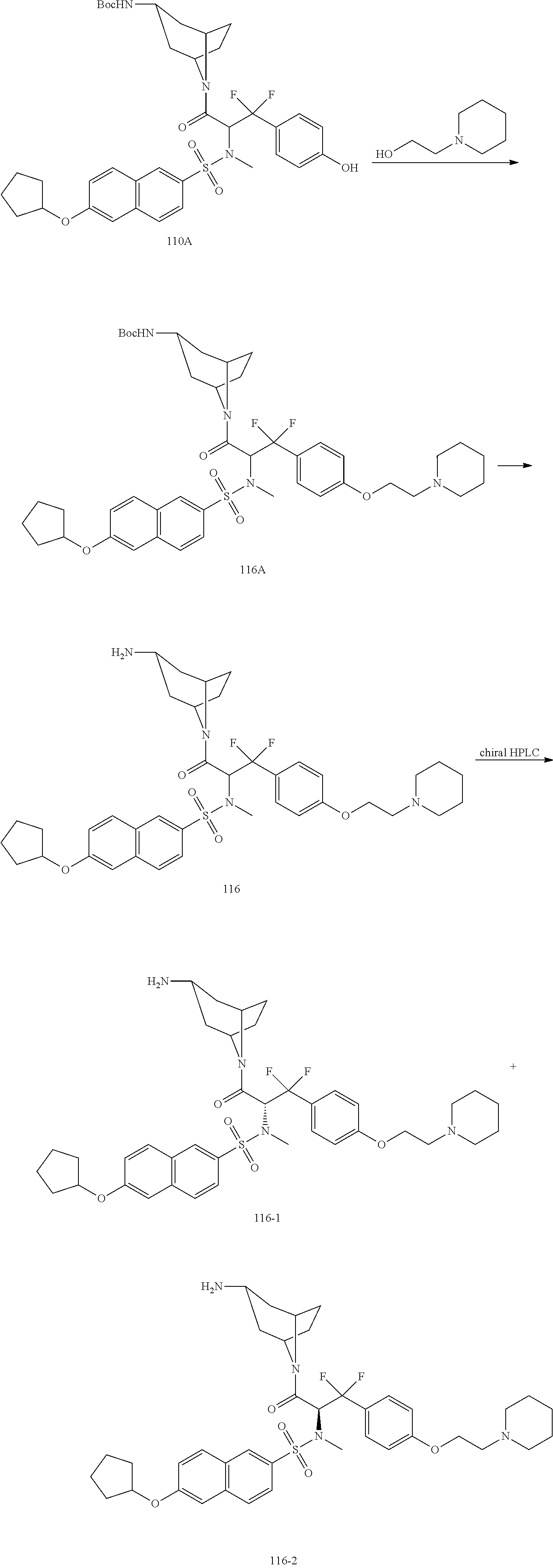

Compounds 116A and 116 were synthesized by employing the procedures described for Compounds 115A and 6 using 2-(piperidin-1-yl)ethan-1-ol and Compound 116A in lieu of 2-morpholinoethan-1-ol and Compound 6C.

Compound 116A: LC-MS (ESI) m/z: 825 [M+H]⁺.

Compound 116 was separated with chiral HPLC to give Compound 116-1 and Compound 116-2.

Compound 116-1: LC-MS (ESI) m/z: 725 [M+H]⁺; ¹H-NMR (CD₃OD, 400 MHz): δ (ppm) 1.23-2.00 (m, 24H), 2.44-2.46 (m, 4H), 2.65-2.69 (m, 2H), 3.02-3.18 (m, 3H), 3.96-4.01 (m, 2H), 4.42-4.46 (m, 1H), 4.90-4.93 (m, 1H), 5.30-5.52 (m, 1H), 6.79-6.87 (m, 2H), 7.12-7.16 (m, 1H), 7.21-7.35 (m, 3H), 7.43-7.56 (m, 1H), 7.74-7.83 (m, 2H), 8.09-8.14 (m, 1H). Chiral separation condition: n-Hexane/EtOH contained 0.1% DEA, 40/60; AY-H (4.6×250 mm, 5 μm); 14.51 minute.

Compound 116-2: LC-MS (ESI) m/z: 725 [M+H]⁺; ¹H-NMR (CD₃OD, 400 MHz): δ (ppm) 1.25-2.00 (m, 24H), 2.44-2.46 (m, 4H), 2.65-2.69 (m, 2H), 3.02-3.18 (m, 3H), 3.96-4.01 (m, 2H), 4.42-4.46 (m, 1H), 4.89-4.92 (m, 1H), 5.30-5.45 (m, 1H), 6.79-6.88 (m, 2H), 7.12-7.16 (m, 1H), 7.21-7.35 (m, 3H), 7.44-7.54 (m, 1H), 7.74-7.83 (m, 2H), 8.09-8.14 (m, 1H). Chiral separation condition: n-Hexane/EtOH contained 0.1% DEA, 40/60; AY-H (4.6×250 mm, 5 μm); 7.58 minute.

### Example 117

### Synthesis of (R)—N-(3-(4-aminopiperidin-1-yl)-1-(4-(5-chlorothiophen-2-yl)phenyl)-1,1-difluoro-3-oxopropan-2-yl)-4-(cyclohexylmethoxy)benzenesulfonamide (Compound 117-1), and (S)—N-(3-(4-aminopiperidin-1-yl)-1-(4-(5-chlorothiophen-2-yl)phenyl)-1,1-difluoro-3-oxopropan-2-yl)-4-(cyclohexylmethoxy) benzenesulfonamide (Compound 117-2)

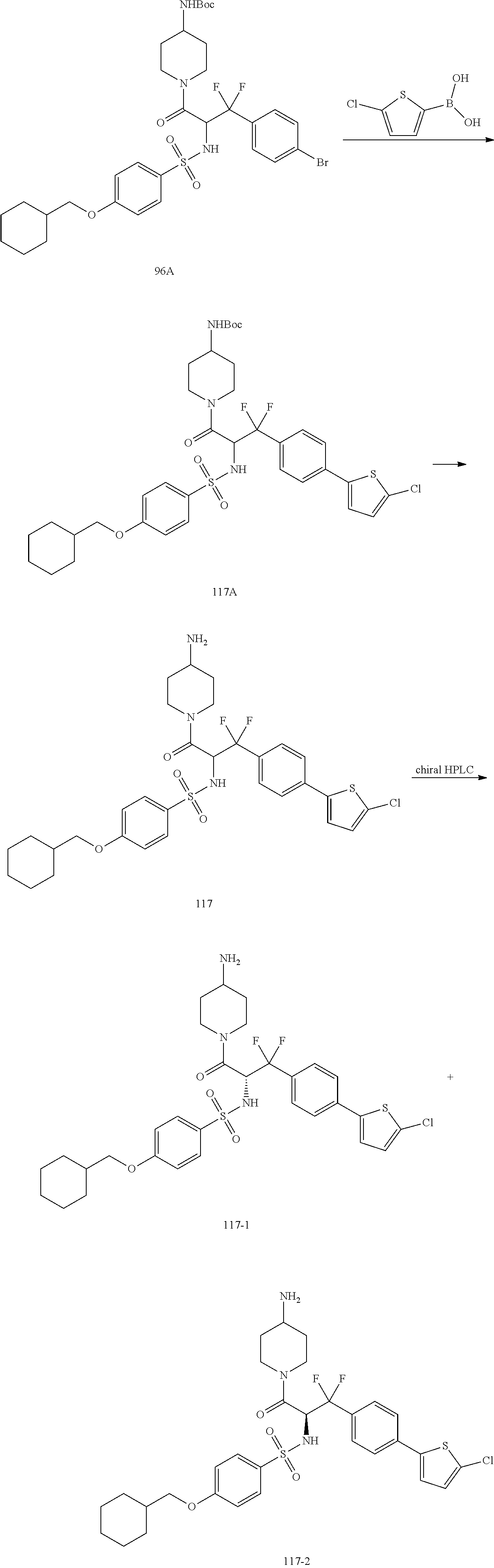

Compounds 117A and 117 were synthesized by employing the procedures described for Compounds 14 and 6 using 5-chlorothiophen-2-ylboronic acid, Compounds 96A, and 117A in lieu of Compounds 14B, 14A, and 6C.

Compound 117A: LC-MS (ESI) m/z: 752 [M+H]⁺.

Compound 117 was separated with chiral HPLC to afford Compound 117-1 and Compound 117-2.

Compound 117-1: LC-MS (ESI) m/z: 652 [M+H]⁺; ¹H-NMR (CD₃OD, 400 MHz): δ (ppm) 1.03-1.39 (m, 7H), 1.69-1.86 (m, 8H), 2.47-2.72 (m, 1H), 2.85-2.90 (m, 1H), 3.01-3.07 (m, 1H), 3.66-3.72 (m, 2H), 3.96-4.04 (m, 1H), 4.20-4.37 (1H), 4.83-4.84 (m, 1H), 6.87 (d, J=8.8 Hz, 2H), 7.04 (d, J=3.6 Hz, 1H), 7.34-7.35 (m, 1H), 7.41-7.47 (m, 2H), 7.52-7.56 (m, 4H). Chiral separation condition: MeOH contained 0.5% NH₄OH; (R, R)-Whelk-O1 (4.6×250 mm, 5 μm); retention time: 7.1 minute.

Compound 117-2: LC-MS (ESI) m/z: 652 [M+H]⁺; ¹H-NMR (CD₃OD, 400 MHz): δ (ppm) 1.04-1.36 (m, 7H), 1.71-1.86 (m, 8H), 2.47-2.72 (m, 1H), 2.85-2.90 (m, 1H), 3.01-3.07 (m, 1H), 3.66-3.71 (m, 2H), 3.96-4.04 (m, 1H), 4.20-4.37 (1H), 4.83-4.84 (m, 1H), 6.87 (d, J=8.8 Hz, 2H), 7.04 (d, J=3.6 Hz, 1H), 7.34-7.35 (m, 1H), 7.41-7.47 (m, 2H), 7.54-7.56 (m, 4H). Chiral separation condition: MeOH contained 0.5% NH₄OH; (R, R)-Whelk-O1 (4.6×250 mm, 5 μm); retention time: 8.74 minute.

### Example 118

### Synthesis of N-((2R)-3-(3-amino-8-azabicyclo[3.2.1]octan-8-yl)-1-(3-butoxy-4-chlorophenyl)-1,1-difluoro-3-oxopropan-2-yl)-6-(cyclopentyloxy)naphthalene-2-sulfonamide (Compound 118-1), and N-((2S)-3-(3-amino-8-azabicyclo[3.2.1]octan-8-yl)-1-(3-butoxy-4-chlorophenyl)-1,1-difluoro-3-oxopropan-2-yl)-6-(cyclopentyloxy) naphthalene-2-sulfonamide (Compound 118-2)

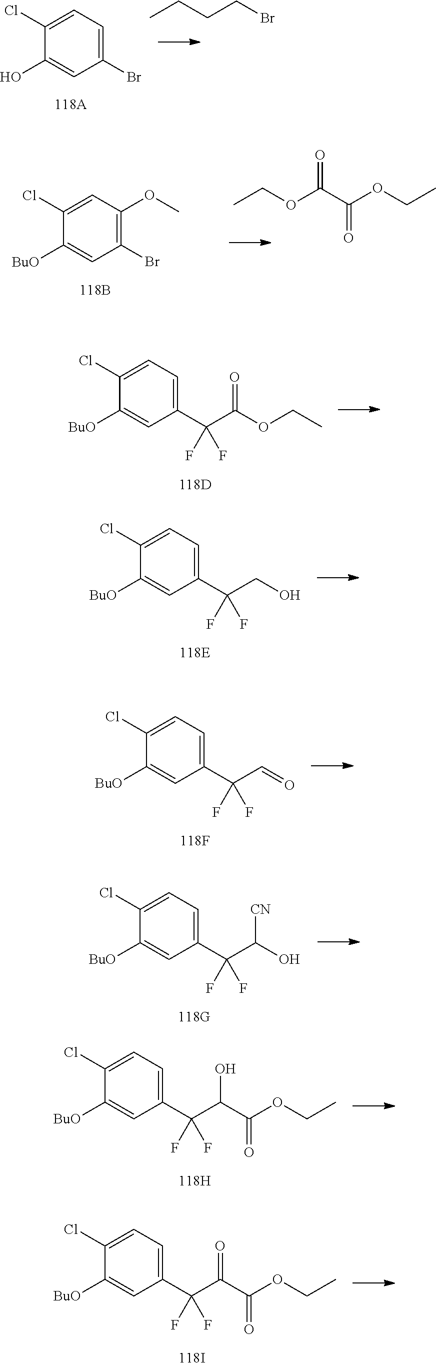

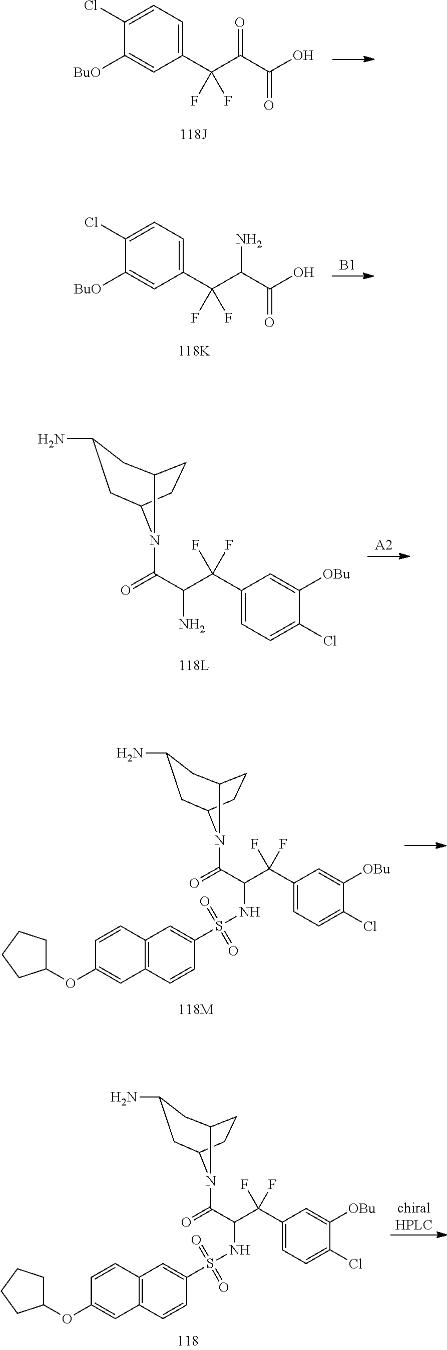

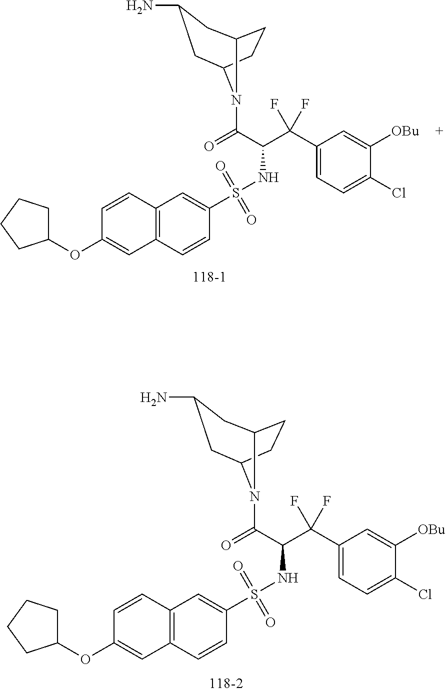

Compounds 118B, 118C, 118D, 118E, 118F, 118G, 118H, 118I, 118J, 118K, 118L, 118M, and 118 were synthesized by employing the procedures described for Compounds 4D, 9B, Compound B2-3, Compound 51E, Compound B6-3, Compounds 45F, 45G, Compound B6-3, Compounds 45I, 80A, Compound B4, Compounds 2D, and 1 using Compounds 118A using K₂CO₃ as base at 80° C., 118B, 118C, 118D, 118E, 118F, 118G, 118H, 118I, 118J, 118K, Compound B1, Compound 118L, Compound A2, and Compound 118M in lieu of Compounds 4C using Cs₂CO₃ as base at room temperature, 9A, Compound B2-2, Compound 51D, Compound B6-2, Compounds 45E, 45F, Compound B6-2, Compounds 45H, 51J, Compounds B2-6, B4-1, B2, Compounds 2C, and 1A.

Compound 118B: ¹H-NMR (CDCl₃, 400 MHz): δ (ppm) 0.98 (t, J=7.6 Hz, 3H), 1.49-1.55 (m, 2H), 1.79-1.83 (m, 2H), 3.99 (t, J=6.4 Hz, 2H), 6.97-7.03 (m, 2H), 7.19 (d, J=8.4 Hz, 1H).

Compound 118C: LC-MS (ESI) m/z: 285[M+H]⁺; ¹H-NMR (CDCl₃, 400 MHz): δ (ppm) 0.98 (t, J=7.6 Hz, 3H), 1.42 (t, J=7.2 Hz, 3H), 1.48-1.55 (m, 2H), 1.79-1.86 (m, 2H), 4.01 (t, J=6.4 Hz, 2H), 4.44 (q, J=7.2 Hz, 2H), 7.01-7.05 (m, 2H), 7.99 (d, J=8.0 Hz, 1H).

Compound 118D: LC-MS (ESI) m/z: non-ionizable compound under routine conditions used.

Compound 118E: ¹H-NMR (CDCl₃, 400 MHz): δ (ppm) 0.99 (t, J=7.2 Hz, 3H), 1.51-1.55 (m, 2H), 1.82-1.86 (m, 2H), 1.94 (t, J=7.2 Hz, 1H), 3.95-4.00 (m, 2H), 4.06 (t, J=6.4 Hz, 2H), 7.03-7.05 (m, 2H), 7.42 (d, J=8.0 Hz, 1H).

Compound 118F was used directly in the next step without further purification.

Compound 118G: LC-MS (ESI) m/z: non-ionizable compound under routine conditions used.

Compound 118H: LC-MS (ESI) m/z: 337 [M+H]⁺; ¹H-NMR (CDCl₃, 400 MHz): δ (ppm) 0.91-0.99 (m, 3H), 1.25-1.31 (m, 3H), 1.50-1.56 (m, 2H), 1.80-1.86 (m, 2H), 3.29 (d, J=7.6 Hz, 1H), 4.02-4.07 (m, 2H), 4.27-4.31 (m, 2H), 4.47-4.54 (m, 1H), 6.97-7.03 (m, 2H), 7.40 (d, J=8.4 Hz, 1H).

Compound 1181: ¹H-NMR (CDCl₃, 400 MHz): δ (ppm) 0.91-0.99 (m, 3H), 1.29-1.35 (m, 3H), 1.54-1.60 (m, 2H), 1.80-1.88 (m, 2H), 4.02-4.25 (m, 4H), 7.09-7.13 (m, 2H), 7.42 (d, J=8.4 Hz, 1H).

Compound 118J: LC-MS (ESI) m/z: 305 [M−H]⁻.

Compound 118K: LC-MS (ESI) m/z: non-ionizable compound under routine conditions used.

Compound 118L: LC-MS (ESI) m/z: non-ionizable compound under routine conditions used.

Compound 118M: LC-MS (ESI) m/z: 812 [M+Na]⁺.

Compound 118 was separated by chiral HPLC to give Compound 118-1 and Compound 118-2.

Compound 118-1: LC-MS (ESI) m/z: 690 [M+H]⁺; ¹H-NMR (CD₃OD, 400 MHz): δ (ppm) 0.98-1.05 (m, 3H), 1.39-2.21 (m, 20H), 3.55-3.89 (m, 3H), 4.48-4.90 (m, 3H), 5.00-5.03 (m, 1H), 6.72-7.04 (m, 2H), 7.13-7.48 (m, 4H), 7.62-7.70 (m, 2H), 8.02-8.23 (m, 1H). Chiral separation condition: MeOH contained 0.5% NH₄OH; IC (150×4.6 mm, 5 μm); retention time: 6.95 minute.

Compound 118-2: LC-MS (ESI) m/z: 690 [M+H]⁺; ¹H-NMR (CD₃OD, 400 MHz): δ (ppm) 0.95-1.03 (m, 3H), 1.38-2.22 (m, 20H), 3.54-3.91 (m, 3H), 4.47-4.85 (m, 3H), 5.01-5.03 (m, 1H), 6.72-7.05 (m, 2H), 7.13-7.48 (m, 4H), 7.62-7.70 (m, 2H), 8.02-8.23 (m, 1H). Chiral separation condition: MeOH contained 0.5% NH₄OH; IC (150×4.6 mm, 5 μm); retention time: 5.51 minute.

### Example 119

### Synthesis of (R)—N-(3-(4-aminopiperidin-1-yl)-1-(4-bromophenyl)-1,1-difluoro-3-oxopropan-2-yl)-5-bromo-6-isopropoxynaphthalene-2-sulfonamide (Compound 119-1), and (S)—N-(3-(4-aminopiperidin-1-yl)-1-(4-bromophenyl)-1,1-difluoro-3-oxopropan-2-yl)-5-bromo-6-isopropoxynaphthalene-2-sulfonamide (Compound 119-2)

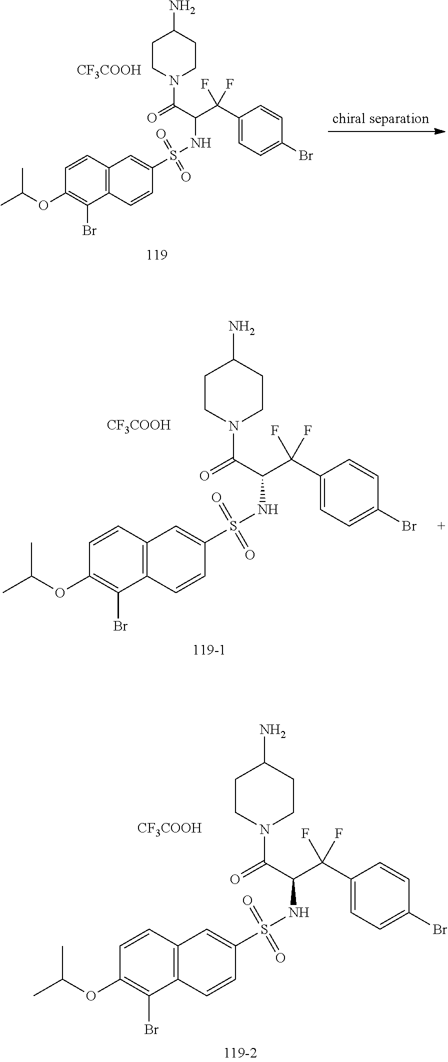 [SMILES: CC(C)Oc1ccc2cc(S(=O)(=O)NC(C(=O)N3CCC(N)CC3)C(F)(F)c3ccc(Br)cc3)ccc2c1Br ; "CF3COOH" ; CC(C)Oc1ccc2cc(S(=O)(=O)N[C@H](C(=O)N3CCC(N)CC3)C(F)(F)c3ccc(Br)cc3)ccc2c1Br ; "CF3COOH" ; CC(C)Oc1ccc2cc(S(=O)(=O)N[C@@H](C(=O)N3CCC(N)CC3)C(F)(F)c3ccc(Br)cc3)ccc2c1Br ; "CF3COOH"]

Compounds 119-1 and 119-2 were prepared by similar methods disclosed herein, such as, for example, by the method described in Example 27, and as depicted in the scheme above.

### Example 120

### Synthesis of N-((2R)-3-(3-amino-8-azabicyclo[3.2.1]octan-8-yl)-1-(3-butoxy-4-chlorophenyl)-1,1-difluoro-3-oxopropan-2-yl)-4-(cyclohexylmethoxy) benzenesulfonamide (Compound 120-1), and N-((2S)-3-(3-amino-8-azabicyclo[3.2.1]octan-8-yl)-1-(3-butoxy-4-chlorophenyl)-1,1-difluoro-3-oxopropan-2-yl)-4-(cyclohexylmethoxy)benzenesulfonamide (Compound 120-2)

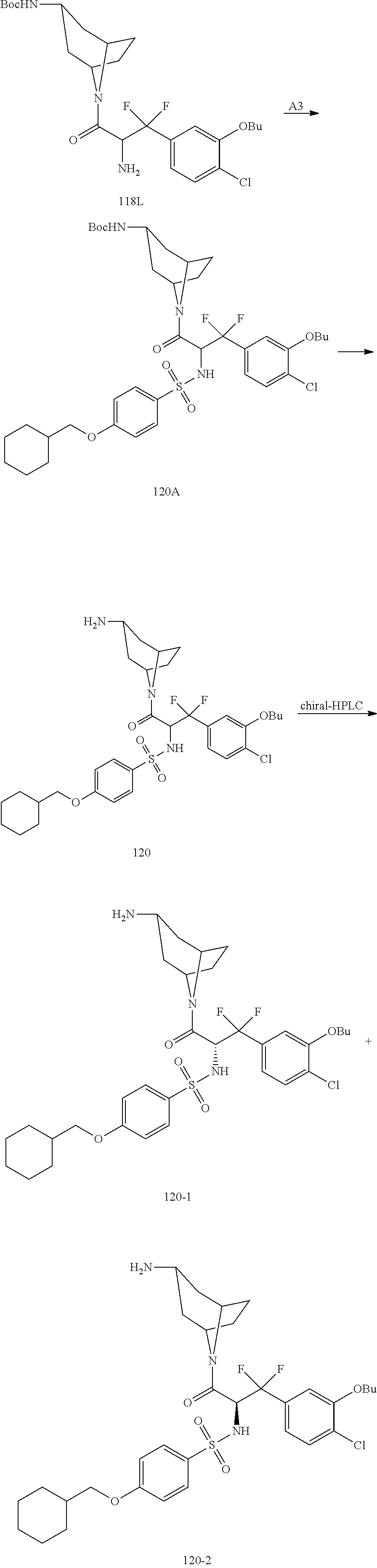

Compounds 120A and 120 were synthesized by employing the procedures described for Compounds 2D and 6 using Compounds 118L, Compound A3, and 120A in lieu of Compound B2, Compounds 2C, and 6C.

Compound 120A: LC-MS (ESI) m/z: 768 [M+H]⁺.

Compound 120 was separated with chiral HPLC to give Compound 120-1 and Compound 120-2.

Compound 120-1: LC-MS (ESI) m/z: 668 [M+H]⁺; ¹H-NMR (CD₃OD, 400 MHz): δ (ppm) 0.98-1.04 (m, 3H), 1.39-1.14 (m, 6H), 1.55-1.70 (m, 3H), 1.71-2.17 (m, 14H), 3.63-3.88 (m, 5H), 4.69-4.81 (m, 3H), 6.82-7.52 (m, 7H). Chiral separation condition: n-hexane/EtOH contained 0.1% DEA, 70/30; S, S-WHELK-O1 (250×4.6 mm, 5 μm); retention time: 11.84 minute.

Compound 120-2: LC-MS (ESI) m/z: 668 [M+H]⁺; ¹H-NMR (CD₃OD, 400 MHz): δ (ppm) 0.98-1.05 (m, 3H), 1.39-1.15 (m, 6H), 1.57-1.71 (m, 3H), 1.72-2.17 (m, 14H), 3.63-3.89 (m, 5H), 4.71-4.81 (m, 3H), 6.82-7.52 (m, 7H). Chiral separation condition: n-hexane/EtOH contained 0.1% DEA, 70/30; S, S-WHELK-O1 (250×4.6 mm, 5 μm); retention time: 10.49 minute.

### Example 121

### Synthesis of N-((2R)-3-(3-amino-8-azabicyclo[3.2.1]octan-8-yl)-1-(4-(4-chlorophenoxy)phenyl)-1,1-difluoro-3-oxopropan-2-yl)-6-(cyclopentyloxy) naphthalene-2-sulfonamide (Compound 121-1), and N-((2S)-3-(3-amino-8-azabicyclo[3.2.1]octan-8-yl)-1-(4-(4-chlorophenoxy)phenyl)-1,1-difluoro-3-oxopropan-2-yl)-6-(cyclopentyloxy)naphthalene-2-sulfonamide (Compound 121-2)

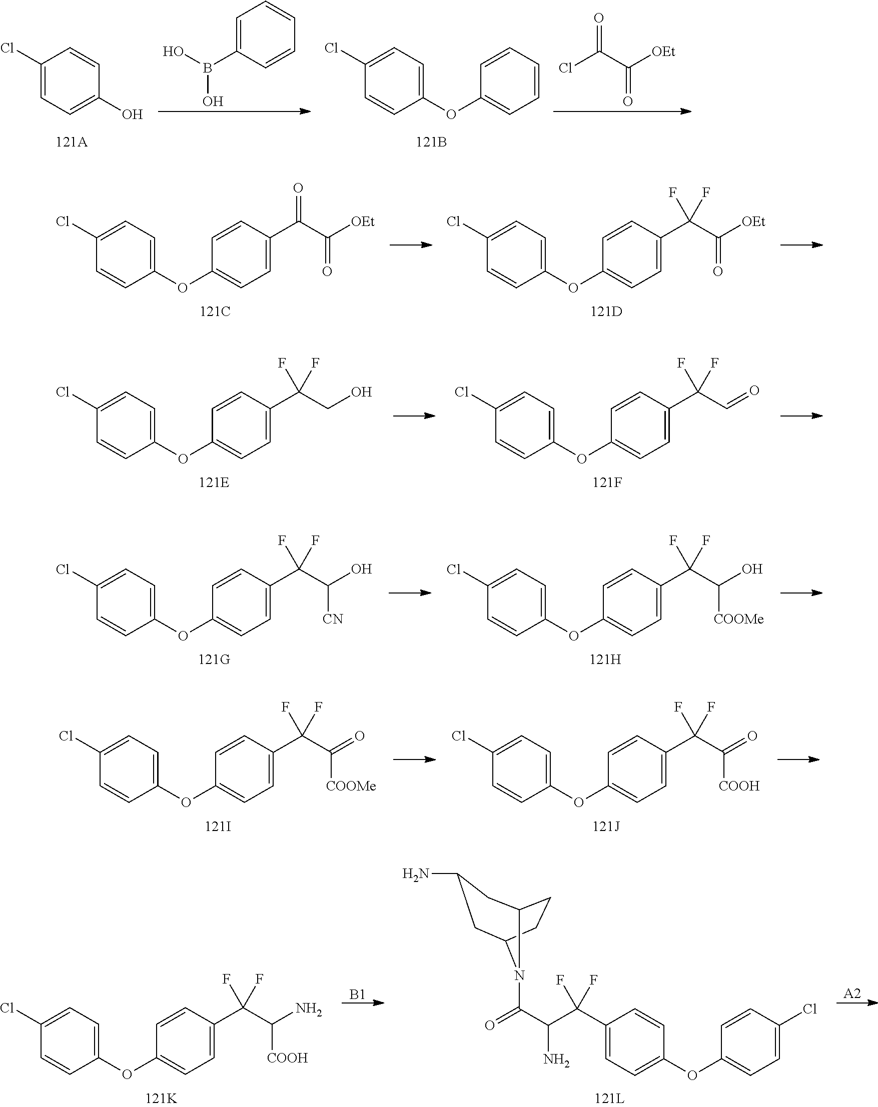

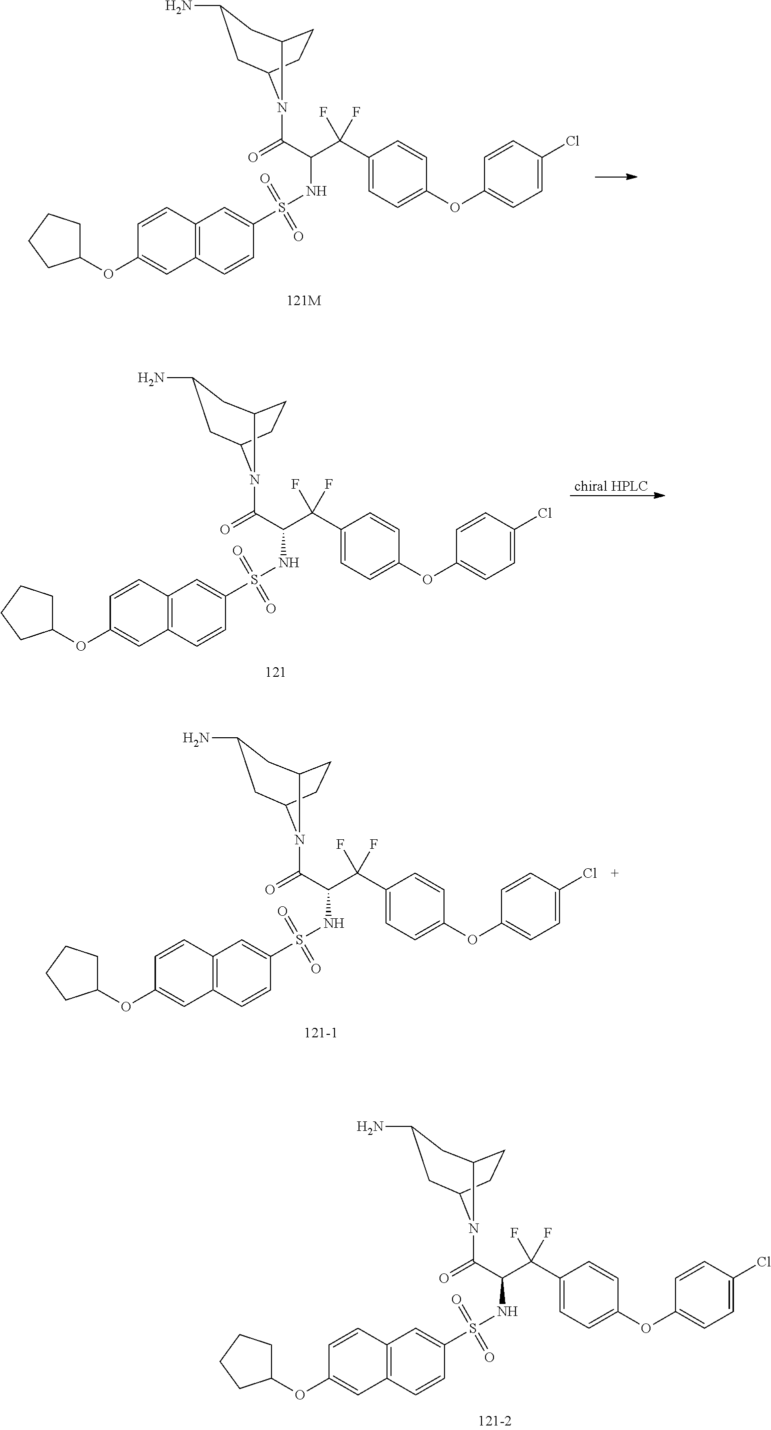

To a solution of Compound 121A (20 g, 156 mmol) and phenylboronic acid (38 g, 312 mmol) in dry 1,2-dichloroethane (800 mL) was added 4A molecular sieves powder (156 g) and dry triethylamine (108 mL, 780 mmol) at 0° C. The reaction mixture was stirred at 0° C. under anhydrous condition for 7 hours, warmed up to 10° C., and stirred for 48 hours. The mixture was poured into petroleum ether (1 L) and filtered through Celite. The filtration was concentrated under reduced pressure. The residue was purified by silica gel column chromatography to give Compound 121B. ¹H-NMR (CDCl₃, 400 MHz): δ (ppm) 6.93-6.96 (m, 2H), 6.99-7.01 (m, 2H), 7.11 (t, J=7.6 Hz, 1H), 7.27-7.33 (m, 2H), 7.35 (t, J=9.2 Hz, 2H).

Compounds 121C, 121D, 121E, 121F, 121G, 121H, 121I, 121J, 121K, 121L, 121M, and 121 were synthesized by employing the procedures described for Compounds B2-2, B2-3, Compound 51E, Compound B6-3, Compounds 45F, 45G, Compound B6-3, Compounds 451, 80A, Compound B4, Compounds 2D, and 1 using Compounds 121B, 121C, 121D using EtOH as solvent, 121E, 121F, 121G in MeOH, 121H, 121I, 121J, 121K, Compound B1, Compound 121L, Compound A2, and Compound 121M in lieu of Compounds B2-1, B2-2, Compound 51D using MeOH as solvent, Compound B6-2, Compounds 45E, 45F in EtOH, Compound B6-2, Compounds 45H, 51J, Compounds B2-6, B4-1, B2, Compounds 2C, and 1A.

Compound 121C: LC-MS (ESI) m/z: 305 [M+H]⁺.

Compound 121D: ¹H-NMR (CDCl₃, 400 MHz): δ (ppm) 1.30 (t, J=7.2 Hz, 3H), 4.28-4.34 (m, 2H), 6.97-7.03 (m, 4H), 7.32-7.35 (m, 2H), 7.56 (d, J=8.8 Hz, 2H).

Compound 121E: LC-MS (ESI) m/z: 265 [M−F]⁺; ¹H-NMR (CDCl₃, 400 MHz): δ (ppm) 1.91 (t, J=7.2 Hz, 1H), 3.93-4.01 (m, 2H), 6.96-7.03 (m, 4H), 7.31-7.33 (m, 2H), 7.47 (d, J=8.8 Hz, 2H).

Compound 121F was used directly in the next step without further purification. LC-MS (ESI) m/z: non-ionizable compound under routine conditions used.

Compound 121G: LC-MS (ESI) m/z: 290 [M−F]⁺; ¹H-NMR (CDCl₃, 400 MHz): δ (ppm) 4.27 (s, 1H), 4.78 (m, 1H), 6.98-7.02 (m, 2H), 7.05 (d, J=8.4 Hz, 2H), 7.33-7.37 (m, 2H), 7.53 (d, J=8.8 Hz, 2H).

Compound 121H: LC-MS (ESI) m/z: 365 [M+Na]⁺; ¹H-NMR (CDCl₃, 400 MHz): δ (ppm) 3.20 (d, J=8.0 Hz, 1H), 3.85 (s, 3H), 4.50-4.57 (m, 1H), 6.97-7.02 (m, 4H), 7.32 (d, J=8.8 Hz, 2H). 7.44 (d, J=8.4 Hz, 2H).

Compound 1211: LC-MS (ESI) m/z: 381 [M+H₂O+Na]⁺; ¹H-NMR (CDCl₃, 400 MHz): δ (ppm) 3.96 (s, 3H), 6.98 (t, J=9.2 Hz, 4H), 7.32 (d, J=8.8 Hz, 2H), 7.54 (d, J=8.8 Hz, 2H).

Compound 121J: LC-MS (ESI) m/z: 367 [M+H₂O+Na]⁺; ¹H-NMR (DMSO-d₆, 400 MHz): δ (ppm) 7.09-7.13 (m, 4H), 7.46-7.48 (m, 2H), 7.62 (d, J=8.4 Hz, 2H).

Compound 121K: LC-MS (ESI) m/z: 328 [M+H]⁺.

Compound 121L: LC-MS (ESI) m/z: 536 [M+H]⁺.

Compound 121M: LC-MS (ESI) m/z: 810 [M+H]⁺.

Compound 121 was separated with chiral HPLC to give Compound 121-1 and Compound 121-2.

Compound 121-1: LC-MS (ESI) m/z: 710 [M+H]⁺; ¹H-NMR (CD₃OD, 400 MHz): δ (ppm) 0.59-1.47 (m, 3H), 1.59-2.09 (m, 13H), 3.37-3.66 (m, 1H), 4.22-4.60 (m, 3H), 4.86-4.90 (m, 1H), 6.55 (d, J=8.4 Hz, 1H), 6.71 (d, J=15.2 Hz, 1H), 6.84-6.92 (m, 2H), 7.11-7.30 (m, 5H), 7.40-7.54 (m, 2H), 7.67-7.77 (m, 2H), 8.03-8.09 (m, 1H). Chiral separation condition: MeOH contained 0.5% NH₄OH; IC (150×4.6 mm, 5 μm); retention time: 6.59 minute.

Compound 121-2: LC-MS (ESI) m/z: 710 [M+H]⁺; ¹H-NMR (CD₃OD, 400 MHz): δ (ppm) 0.73-1.36 (m, 3H), 1.58-2.18 (m, 13H), 3.50-3.76 (m, 1H), 4.33-4.71 (m, 3H), 4.99-5.03 (m, 1H), 6.66 (d, J=8.4 Hz, 1H), 6.83 (d, J=8.8 Hz, 1H), 6.95-7.01 (m, 2H), 7.22-7.40 (m, 5H), 7.50-7.65 (m, 2H), 7.77-7.88 (m, 2H), 8.13-8.20 (m, 1H). Chiral separation condition: MeOH contained 0.5% NH₄OH; IC (150×4.6 mm, 5 μm); retention time: 9.25 minute.

### Example 122

### Synthesis of N—((R)-3-((R)-3-aminopiperidin-1-yl)-1-(4-bromophenyl)-1,1-difluoro-3-oxopropan-2-yl)-6-(cyclopentyloxy)naphthalene-2-sulfonamide (Compound 122-1), and N—((S)-3-((R)-3-aminopiperidin-1-yl)-1-(4-bromophenyl)-1,1-difluoro-3-oxopropan-2-yl)-6-(cyclopentyloxy)naphthalene-2-sulfonamide (Compound 122-2)

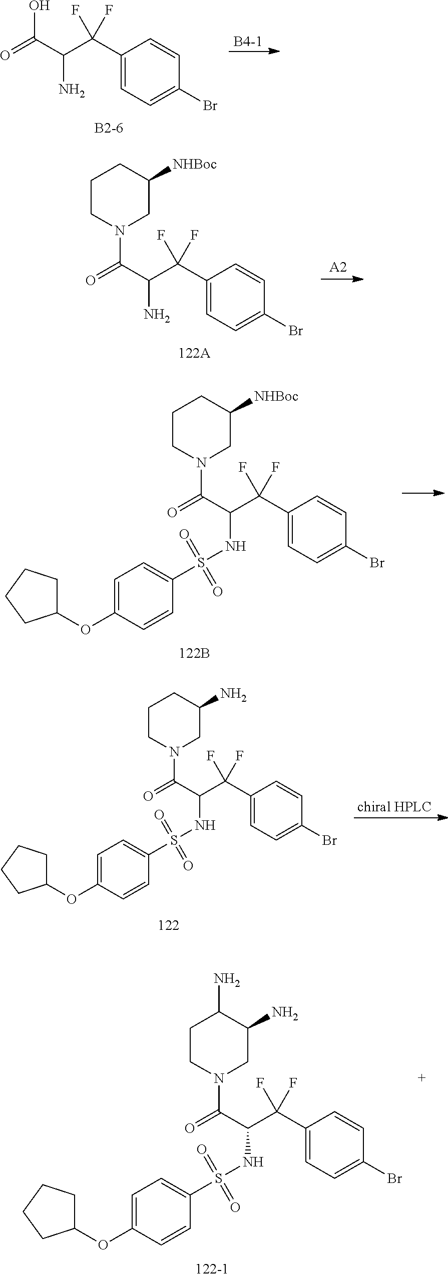

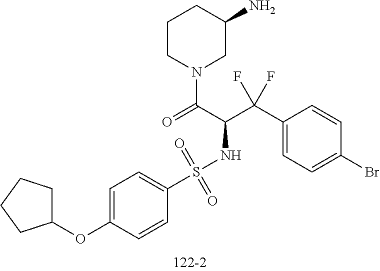 [SMILES: N[C@@H]1CCCN(C(=O)[C@H](NS(=O)(=O)c2ccc(OC3CCCC3)cc2)C(F)(F)c2ccc(Br)cc2)C1]

Compounds 122A, 122B, and 122 were synthesized by employing the procedures described for Compound B4, Compounds 2D, and 1 using (R)-tert-butyl piperidin-3-ylcarbamate, Compound 122A, Compound A2, and Compound 122B in lieu of Compounds B4-1, B2, Compounds 2C, and 1A. Compound 122A: LC-MS (ESI) m/z: 462 [M+H]⁺.

Compound 122B: LC-MS (ESI) m/z: 636[M−Boc+H]⁺.

Compound 122 was separated with chiral HPLC to afford Compound 122-1 and Compound 122-2.

Compound 122-1: LC-MS (ESI) m/z: 636 [M+H]⁺. ¹H NMR (DMSO-d₆, 400 MHz): δ (ppm) 0.82-0.94 (m, 4H), 1.22-1.41 (m, 2H), 1.61-1.79 (m, 8H), 2.01 (s, 3H), 3.59-3.83 (m, 3H), 4.85-4.98 (m, 2H), 7.23 (d, J=8.8 Hz, 1H), 7.36-7.42 (m, 3H), 7.56-7.58 (m, 3H), 7.82-7.85 (m, 1H), 7.91-7.97 (m, 1H), 8.16 (s, 1H). Chiral separation condition: MeOH contained 0.2% Methanol Ammonia; AD-H (250×4.6 mm, 5 μm); retention time: 5.36 minute.

Compound 122-2: LC-MS (ESI) m/z: 636[M+H]⁺. ¹H NMR (DMSO-d₆, 400 MHz): δ (ppm) 0.76-0.90 (m, 2H), 1.14-1.50 (m, 3H), 1.62-1.79 (m, 7H), 2.03-2.13 (m, 3H), 2.19-2.32 (m, 2H), 3.75-3.83 (m, 3H), 4.91-5.00 (m, 2H), 7.23-7.26 (m, 1H), 7.38-7.40 (m, 2H), 7.44 (d, J=8.8 Hz, 1H), 7.58-7.66 (m, 3H), 7.84-7.88 (m, 1H), 7.91-7.98 (m, 1H), 8.19 (s, 1H). Chiral separation condition: MeOH contained 0.2% Methanol Ammonia; AD-H (250×4.6 mm, 5 μm); retention time: 3.31 minute.

### Example 123

### Synthesis of (R)—N-(3-(4-aminopiperidin-1-yl)-1-(4-chloro-3-methoxyphenyl)-1,1-difluoro-3-oxopropan-2-yl)-6-(cyclopentyloxy)naphthalene-2-sulfonamide (Compound 123-1), and (S)—N-(3-(4-aminopiperidin-1-yl)-1-(4-chloro-3-methoxyphenyl)-1,1-difluoro-3-oxopropan-2-yl)-6-(cyclopentyloxy)naphthalene-2-sulfonamide (Compound 123-2)

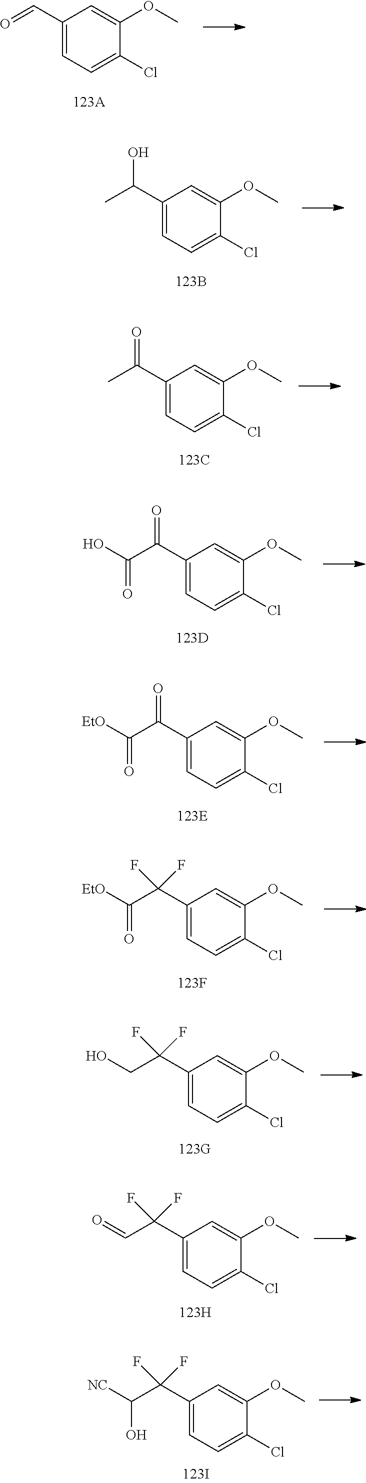

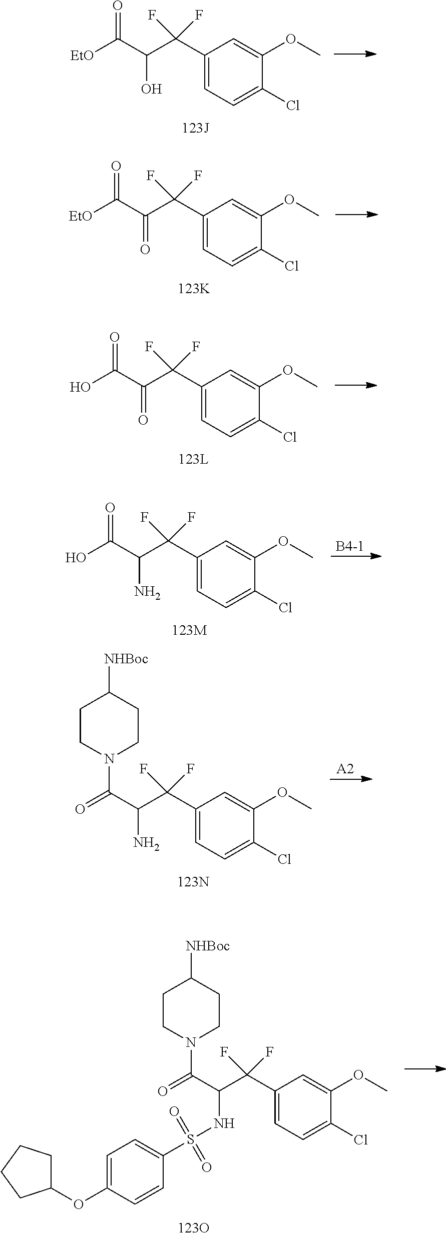

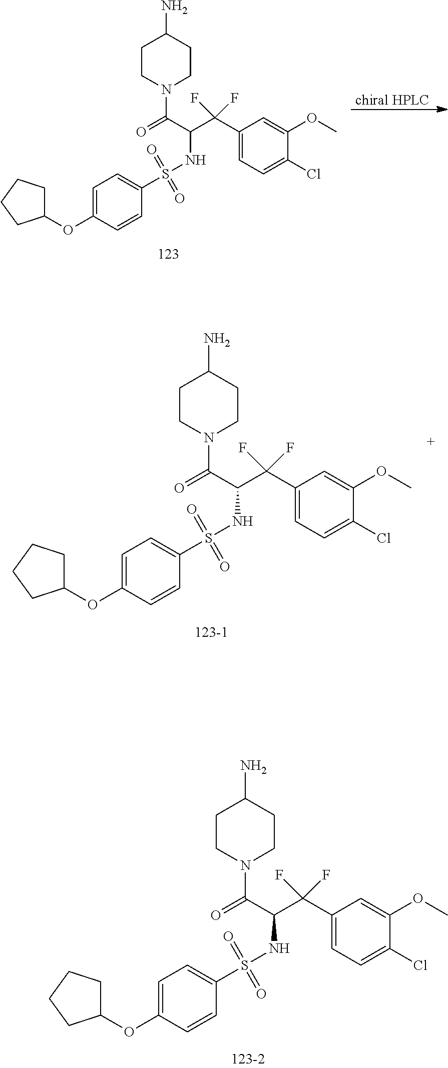

Compounds 123B, 123C, 123D, 123E, 123F, 123G, 123H, 123I, 123J, 123K, 123L, 123M, 123N, 123O, and 123 were synthesized by employing the procedures described for Compound 89B, Compound B6-3, Compounds 42B, 42C, Compound B2-3, Compound 51E, Compound B6-3, Compounds 45F, 45G, Compound B6-3, Compounds 451, 80A, Compound B4, Compounds 1A, and 6 using Compounds 123A at −78° C., 123B, 123C, 123D, 123E, 123F, 123G, 123H, 123I, 123J, 123K, 123L, 123M, 123N, Compound A2, and Compound 123O in lieu of Compound 89A at −30° C., Compound B6-2, Compounds 42A, 42B, Compound B2-2, Compound 51D, Compound B6-2, Compounds 45E, 45F, Compound B6-2, Compounds 4511, 51J, Compounds B2-6, B2, A1, and Compound 6C.

Compound 123B: LC-MS (ESI) m/z: 169 [M−OH]⁺.

Compound 123C: LC-MS (ESI) m/z: 185 [M+H]⁺; ¹H-NMR (CDCl₃, 400 MHz): δ (ppm) 2.60 (s, 3H), 3.97 (s, 3H), 7.44-7.49 (m, 2H), 7.54 (s, 1H).

Compound 123D: LC-MS (ESI) m/z: 213 [M−H]; ¹H-NMR (CDCl₃, 400 MHz): δ (ppm) 3.99 (s, 3H), 5.63 (bs, 1H), 7.53 (d, J=8.4 Hz, 1H), 7.90 (d, J=1.6 Hz, 1H), 8.02 (dd, J₁=2.0 Hz, J2=8.4 Hz, 1H).

Compound 123E: LC-MS (ESI) m/z: 243 [M+H]⁺; ¹H-NMR (CDCl₃, 400 MHz): δ (ppm) 1.43 (t, J=7.2 Hz, 3H), 3.98 (s, 3H), 4.45 (q, J=7.2 Hz, 2H), 7.50 (d, J=8.0 Hz, 1H), 7.55 (dd, J=2.0 Hz, J₂=8.0 Hz, 1H), 7.61 (d, J=1.6 Hz, 1H).

Compound 123F: LC-MS (ESI) m/z: 245 [M−F]⁺; ¹H-NMR (CDCl₃, 400 MHz): δ (ppm) 1.31 (t, J=7.2 Hz, 3H), 3.94 (s, 3H), 4.31 (q, J=7.2 Hz, 2H), 7.14-7.15 (m, 2H), 7.43-7.45 (m, 1H).

Compound 123G: LC-MS (ESI) m/z: 203 [M−F]⁺; ¹H-NMR (CDCl₃, 400 MHz): δ (ppm) 2.08-2.12 (m, 1H), 3.93-4.00 (m, 5H), 7.04-7.07 (m, 2H), 7.43-7.45 (m, 1H).

Compound 123H was used directly for the next step without further purification.

Compound 1231: ¹H-NMR (CDCl₃, 400 MHz): δ (ppm) 3.43 (m, 1H), 3.96 (s, 3H), 4.81-4.85 (m, 1H), 7.11-7.12 (m, 2H), 7.48-7.50 (m, 1H).

Compound 123J: LC-MS (ESI) m/z: 275 [M−F]⁺; ¹H-NMR (CDCl₃, 400 MHz): δ (ppm) 1.30 (t, J=7.2 Hz, 3H), 3.25 (d, J=8.0 Hz, 1H), 3.93 (s, 3H), 4.30 (q, J=7.2 Hz, 2H), 4.48-4.55 (m, 1H), 7.01-7.07 (m, 2H), 7.42 (q, J=8.0 Hz, 1H).

Compound 123K: ¹H-NMR (CDCl₃, 400 MHz): δ (ppm) 1.39 (t, J=7.2 Hz, 3H), 3.93 (s, 3H), 4.41 (q, J=7.2 Hz, 2H), 7.12-7.16 (m, 2H), 7.72 (d, J=8.0 Hz, 1H).

Compound 123L: LC-MS (ESI) m/z: 263 [M−H]; ¹H-NMR (DMSO-d₆, 400 MHz): δ (ppm) 3.89 (s, 3H), 7.20 (dd, J=1.6 Hz, J₂=8.0 Hz, 1H), 7.36 (d, J=1.6 Hz, 1H), 7.56 (d, J=8.0 Hz, 1H).

Compound 123M: LC-MS (ESI) m/z: 266 [M+H]⁺; ¹H-NMR (DMSO-d₆, 400 MHz): δ (ppm) 3.90 (s, 3H), 4.50-4.57 (m, 1H), 7.12 (dd, J=1.6 Hz, J₂=8.0 Hz, 1H), 7.29 (d, J=1.6 Hz, 1H), 7.55 (d, J=8.0 Hz, 1H), 8.76 (bs, 2H).

Compound 123N: LC-MS (ESI) m/z: 448 [M+H]⁺; ¹H-NMR (CDCl₃, 400 MHz): δ (ppm) 0.89-1.35 (m, 2H), 1.44 (s, 9H), 1.92-1.98 (m, 2H), 2.68-3.15 (m, 2H), 3.62-3.84 (m, 2H), 3.91-3.93 (m, 3H), 4.30-4.53 (m, 3H), 6.95-7.03 (m, 2H), 7.40-7.43 (m, 1H).

Compound 123O: LC-MS (ESI) m/z: 622 [M−Boc+1]⁺.

Compound 123 was separated with chiral HPLC to give Compound 123-1 and Compound 123-2

Compound 123-1: LC-MS (ESI) m/z: 622 [M+H]⁺; ¹H-NMR (CD₃OD, 400 MHz): δ (ppm) 1.28-1.52 (m, 2H), 1.71-2.20 (m, 11H), 2.75-3.25 (m, 2H), 3.60-3.77 (m, 3H), 4.17-4.61 (m, 2H), 4.95-5.07 (m, 2H), 6.84-6.91 (m, 1H), 7.00-7.28 (m, 4H), 7.37-7.51 (m, 1H), 7.65-7.87 (m, 2H), 8.06-8.15 (m, 1H). Chiral separation condition: MeOH contained 0.2% NH₄OH; OJ-H (4.6×250 mm, 5 μm); retention time: 4.25 minute.

Compound 123-2: LC-MS (ESI) m/z: 622 [M+H]⁺; ¹H-NMR (CD₃OD, 400 MHz): δ (ppm) 0.23-0.59 (m, 1H), 0.90-1.02 (m, 1H), 1.51-2.07 (m, 11H), 2.41-2.82 (m, 2H), 3.81-3.82 (m, 3H), 3.86-4.16 (m, 2H), 4.91-5.00 (m, 2H), 6.99-7.07 (m, 2H), 7.20-7.33 (m, 3H), 7.54-7.61 (m, 1H), 7.75-7.90 (m, 2H), 8.18-8.22 (m, 1H). Chiral separation condition: MeOH contained 0.2% NH₄OH; OJ-H (4.6×250 mm, 5 μm); retention time: 3.12 minute.

### Example 124

### Synthesis of N-((2R)-3-(3-amino-8-azabicyclo[3.2.1]octan-8-yl)-1-(4-chloro-3-methoxyphenyl)-1,1-difluoro-3-oxopropan-2-yl)-6-(cyclopentyloxy)naphthalene-2-sulfonamide (Compound 124-1), and N-((2S)-3-(3-amino-8-azabicyclo[3.2.1]octan-8-yl)-1-(4-chloro-3-methoxyphenyl)-1,1-difluoro-3-oxopropan-2-yl)-6-(cyclopentyloxy) naphthalene-2-sulfonamide (Compound 124-2)

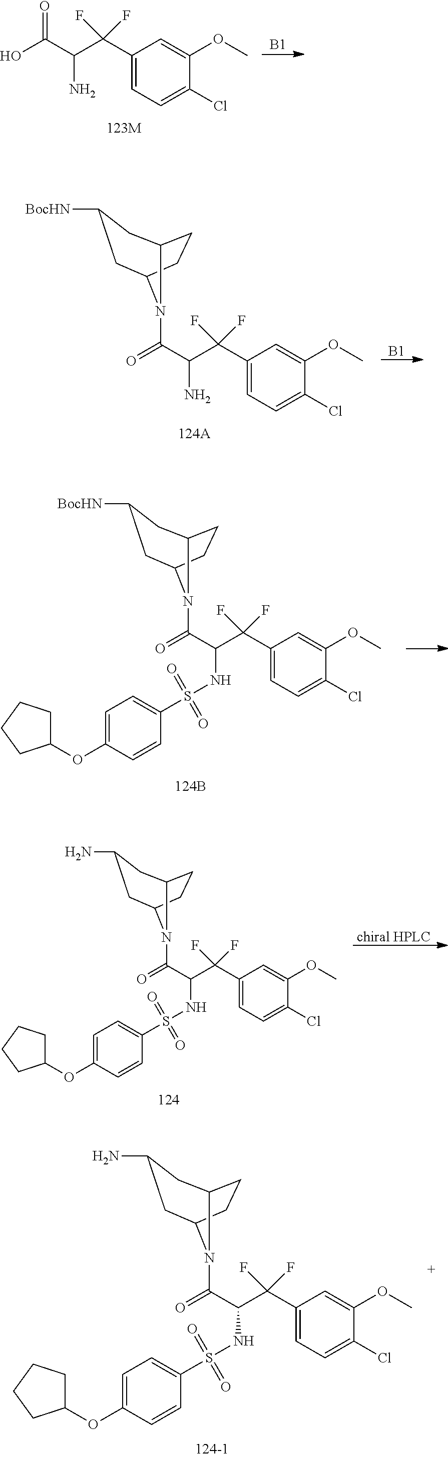

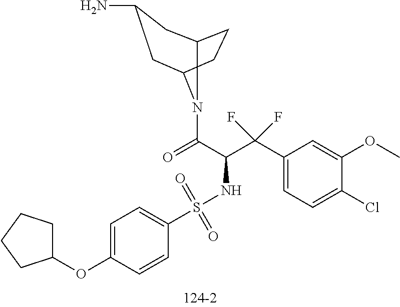 [SMILES: COc1cc(C(F)(F)[C@@H](NS(=O)(=O)c2ccc(OC3CCCC3)cc2)C(=O)N2C3CCC2CC(N)C3)ccc1Cl]

Compounds 124A, 124B, and 124 were synthesized by employing the procedures described for Compound B4, Compounds 1A, and 6 using Compound B1, Compounds 123M, 124A, Compound A2, and Compound 124B in lieu of Compounds B4-1, B2-6, B2, A1, and Compound 6C.

Compound 124A: LC-MS (ESI) m/z: 474 [M+H]⁺; ¹H-NMR (CDCl₃, 400 MHz): δ (ppm) 0.54-1.19 (m, 1H), 1.34-1.55 (m, 11H), 1.83-2.05 (m, 6H), 3.90-4.35 (m, 7H), 4.64-4.78 (m, 1H), 5.30 (s, 1H), 6.96-7.09 (m, 2H), 7.40-7.45 (m, 1H).

Compound 124B: LC-MS (ESI) m/z: 648 [M−Boc+1]⁺.

Compound 124 was separated with chiral HPLC to give Compound 124-1 and Compound 124-2.

Compound 124-1: LC-MS (ESI) m/z: 648 [M+H]⁺; ¹H-NMR (CD₃OD, 400 MHz): δ (ppm) 0.90-2.21 (m, 16H), 3.57-3.81 (m, 4H), 4.41-4.72 (m, 3H), 4.98-5.03 (m, 1H), 6.80-6.89 (m, 1H), 7.03-7.53 (m, 5H), 7.65-7.85 (m, 2H), 8.07-8.16 (m, 1H). Chiral separation condition: MeOH contained 0.5% NH₄OH; IC (4.6×250 mm, 5 μm); retention time: 5.03 minute.

Compound 124-2: LC-MS (ESI) m/z: 648 [M+H]⁺; ¹H-NMR (CD₃OD, 400 MHz): δ (ppm) 0.61-2.08 (m, 16H), 2.83-3.07 (m, 1H), 3.80-3.82 (m, 3H), 4.24-4.42 (m, 2H), 4.63-4.74 (m, 1H), 4.96-5.01 (m, 1H), 6.99-7.10 (m, 2H), 7.20-7.36 (m, 3H), 7.56-7.65 (m, 1H), 7.74-7.89 (m, 2H), 8.18-8.23 (m, 1H). Chiral separation condition: MeOH contained 0.5% NH₄OH; IC (4.6×250 mm, 5 μm); retention time: 4.06 minute.

### Example 125

### Synthesis of N-((2R)-3-(3-amino-8-azabicyclo[3.2.1]octan-8-yl)-1-(4-chloro-3-methoxyphenyl)-1,1-difluoro-3-oxopropan-2-yl)-4-(cyclohexylmethoxy) benzenesulfonamide (Compound 125-1), and N-((2S)-3-(3-amino-8-azabicyclo[3.2.1]octan-8-yl)-1-(4-chloro-3-methoxyphenyl)-1,1-difluoro-3-oxopropan-2-yl)-4-(cyclohexylmethoxy)benzenesulfonamide (Compound 125-2)

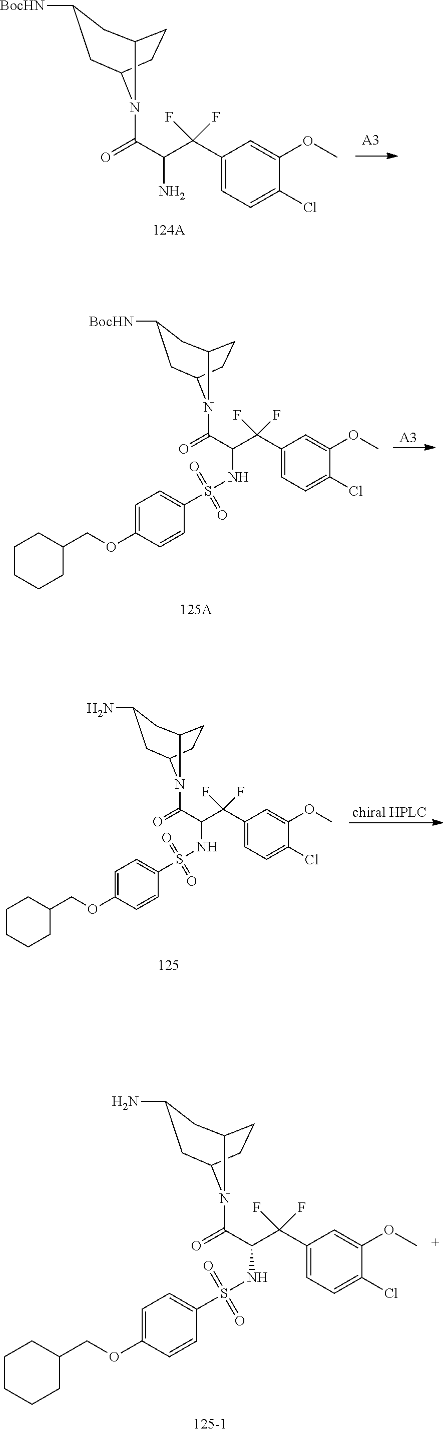

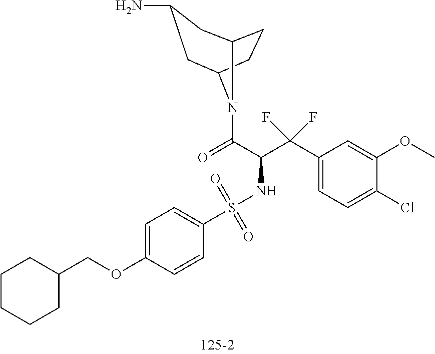 [SMILES: COc1cc(C(F)(F)[C@@H](NS(=O)(=O)c2ccc(OCC3CCCCC3)cc2)C(=O)N2C3CCC2CC(N)C3)ccc1Cl]

Compounds 125A and 125 were synthesized by employing the procedures described for Compounds 1A, and 6 using Compound 124A, Compound A3, and Compound 125A in lieu of Compounds B2, A1, and Compound 6C.

Compound 125A: LC-MS (ESI) m/z: 626 [M−Boc+1]⁺.

Compound 125 was separated with chiral HPLC to give Compound 125-1 and Compound 125-2.

Compound 125-1: LC-MS (ESI) m/z: 626 [M+H]⁺; ¹H-NMR (CD₃OD, 400 MHz): δ (ppm) 1.10-2.20 (m, 19H), 3.63-3.88 (m, 6H), 4.62-4.82 (m, 3H), 6.82-7.09 (m, 4H), 7.29-7.40 (m, 1H), 7.42-7.54 (m, 2H). Chiral separation condition: MeOH contained 0.5% NH₄OH; IC (4.6×250 mm, 5 μm); retention time: 5.24 minute.

Compound 125-2: LC-MS (ESI) m/z: 626 [M+H]⁺; ¹H-NMR (CD₃OD, 400 MHz): δ (ppm) 0.98-1.90 (m, 19H), 3.02-3.13 (m, 1H), 3.83-3.89 (m, 5H), 4.31-4.49 (m, 2H), 4.76-4.69 (m, 1H), 6.90-7.10 (m, 4H), 7.37-7.43 (m, 1H), 7.57-7.62 (m, 2H). Chiral separation condition: MeOH contained 0.5% NH₄OH; IC (4.6×250 mm, 5 μm); retention time: 4.44 minute.

### Example 126

### Synthesis of (R)—N-(3-(4-aminopiperidin-1-yl)-1-(3-butoxy-4-chlorophenyl)-1,1-difluoro-3-oxopropan-2-yl)-6-(cyclopentyloxy)naphthalene-2-sulfonamide (Compound 126-1), and (S)—N-(3-(4-aminopiperidin-1-yl)-1-(3-butoxy-4-chlorophenyl)-1,1-difluoro-3-oxopropan-2-yl)-6-(cyclopentyloxy)naphthalene-2-sulfonamide (Compound 126-2)

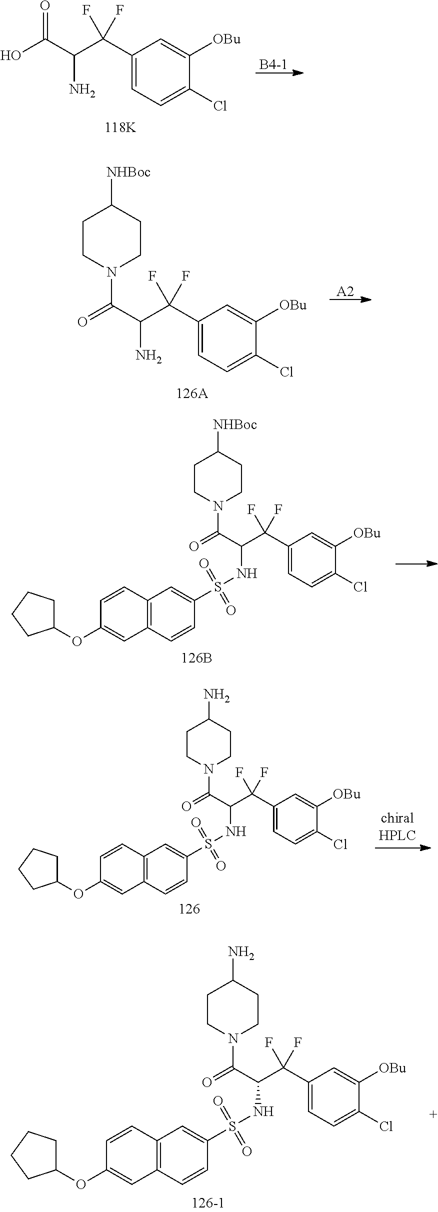

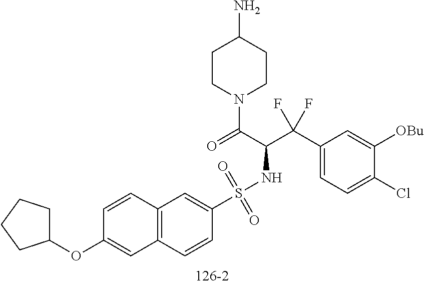

Compounds 126A, 126B, and 126 were synthesized by employing the procedures described for Compound B4, Compounds 2D, and 1 using Compounds 118K, 126A, Compound A2, and Compound 126B in lieu of Compounds B2-6, B2, Compounds 2C, and 1A.

Compound 126A: LC-MS (ESI) m/z: 490 [M+H]⁺.

Compound 126B was used in the next step without further pufification. LC-MS (ESI) m/z: 786 [M+Na]⁺.

Compound 126 was separated with chiral HPLC to give Compound 126-1 and Compound 126-2.

Compound 126-1: LC-MS (ESI) m/z: 664 [M+H]⁺; ¹H-NMR (CD₃OD, 400 MHz): δ (ppm) 0.90-2.20 (m, 18H), 2.71-3.20 (m, 3H), 3.31-4.60 (m, 4H), 4.90-4.97 (m, 3H), 6.70-7.60 (m, 6H), 7.69-7.73 (m, 2H), 7.93-8.14 (m, 1H). Chiral separation condition: MeOH contained 0.2% NH₄OH; IC (150×4.6 mm, 5 μm); retention time: 6.1 minute.

Compound 126-2: LC-MS (ESI) m/z: 664 [M+H]⁺; ¹H-NMR (CD₃OD, 400 MHz): δ (ppm) 0.98-2.28 (m, 18H), 2.73-3.28 (m, 3H), 3.33-4.66 (m, 4H), 4.96-5.02 (m, 3H), 6.75-7.43 (m, 5H), 7.45-7.86 (m, 3H), 8.03-8.23 (m, 1H). Chiral separation condition: MeOH contained 0.2% NH₄OH; IC (150×4.6 mm, 5 μm); retention time: 4.98 minute.

### Example 127

### Synthesis of (R)—N-(3-(4-aminopiperidin-1-yl)-1-(3-butoxy-4-chlorophenyl)-1,1-difluoro-3-oxopropan-2-yl)-4-(cyclohexylmethoxy)benzenesulfonamide (Compound 127-1), and (S)—N-(3-(4-aminopiperidin-1-yl)-1-(3-butoxy-4-chlorophenyl)-1,1-difluoro-3-oxopropan-2-yl)-4-(cyclohexylmethoxy)benzenesulfonamide (Compound 127-2)

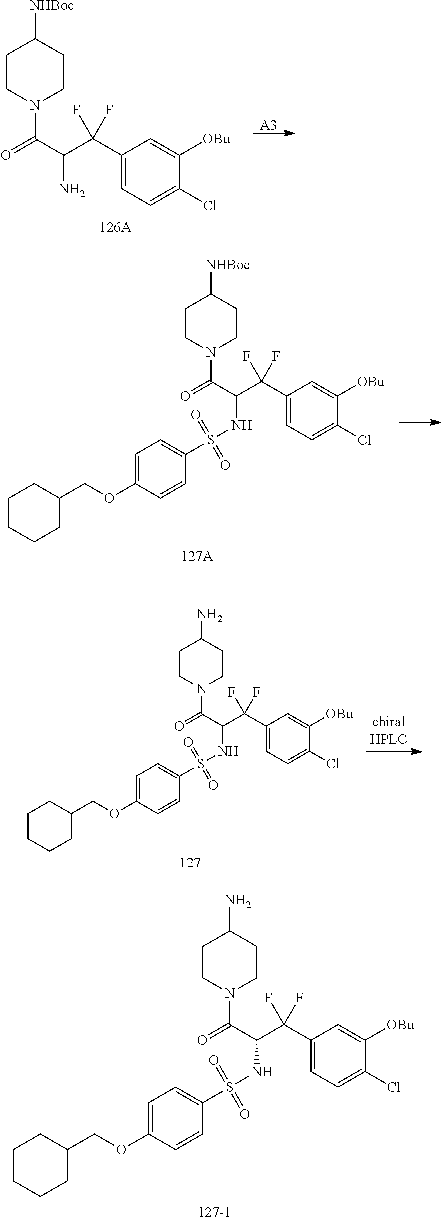

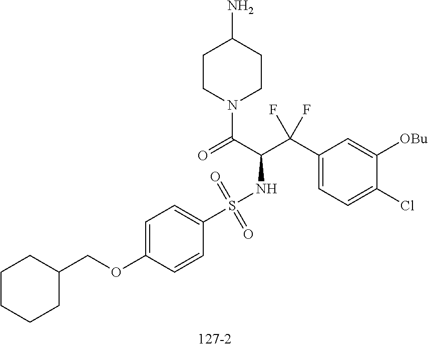

Compounds 127A and 127 were synthesized by employing the procedures described for Compounds 2D and 1 using Compound 126A, Compound A3, and Compound 127A in lieu of Compound B2, Compounds 2C, and 1A.

Compound 127A was used in the next step without further purification. LC-MS (ESI) m/z: 764 [M+Na]⁺.

Compound 127 was separated with chiral HPLC to give Compound 127-1 and Compound 127-2.

Compound 127-1: LC-MS (ESI) m/z: 642 [M+H]⁺; ¹H-NMR (CD₃OD, 400 MHz): δ (ppm) 1.01-2.16 (m, 23H), 3.14-3.42 (m, 2H), 3.79-4.63 (m, 6H), 4.96-5.01 (m, 1H), 6.82-7.04 (m, 4H), 7.40-7.50 (m, 2H), 7.73-7.76 (m, 1H). Chiral separation condition: MeOH contained 0.2% NH₄OH; IC (150×4.6 mm, 5 μm); retention time: 4.64 minute.

Compound 127-2: LC-MS (ESI) m/z: 642 [M+H]⁺; ¹H-NMR (CD₃OD, 400 MHz): δ (ppm) 1.01-2.16 (m, 23H), 3.14-3.42 (m, 2H), 3.79-4.63 (m, 6H), 4.96-5.01 (m, 1H), 6.82-7.04 (m, 4H), 7.40-7.50 (m, 2H), 7.73-7.76 (m, 1H). Chiral separation condition: MeOH contained 0.2% NH₄OH; IC (150×4.6 mm, 5 μm); retention time: 3.61 minute.

### Example 128

### Synthesis of N-((2R)-3-(3-amino-8-azabicyclo[3.2.1]octan-8-yl)-1-(4-bromo-3-methoxyphenyl)-1,1-difluoro-3-oxopropan-2-yl)-4-(cyclohexylmethoxy) benzenesulfonamide (Compound 128-1), and N-((2S)-3-(3-amino-8-azabicyclo[3.2.1]octan-8-yl)-1-(4-bromo-3-methoxyphenyl)-1,1-difluoro-3-oxopropan-2-yl)-4-(cyclohexylmethoxy)benzenesulfonamide (Compound 128-2)

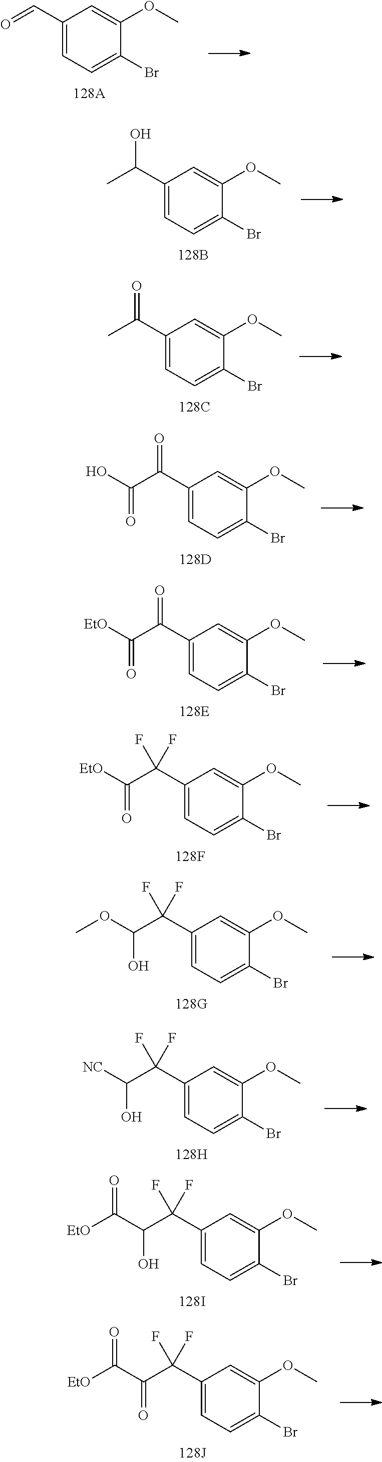

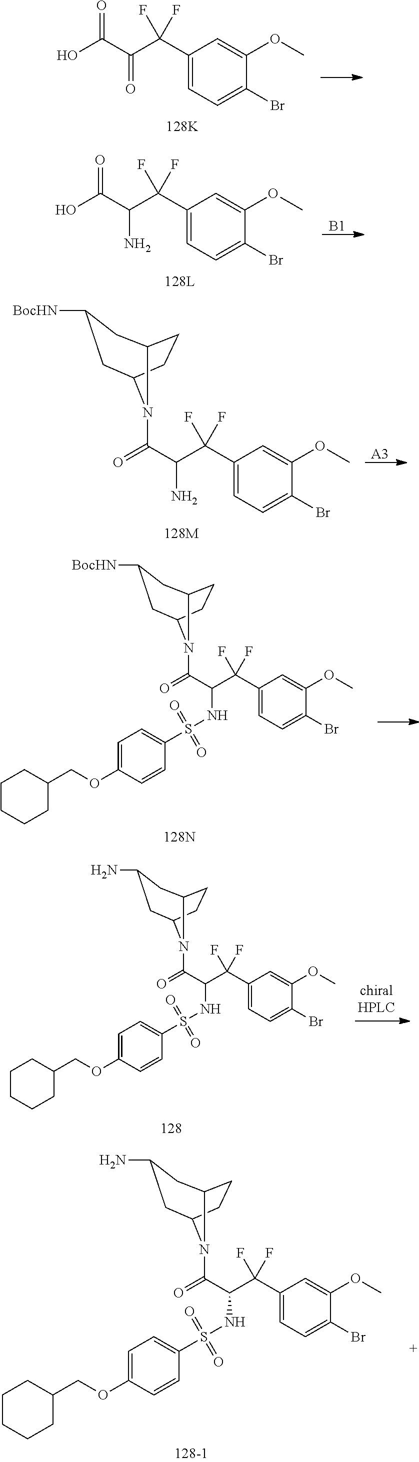

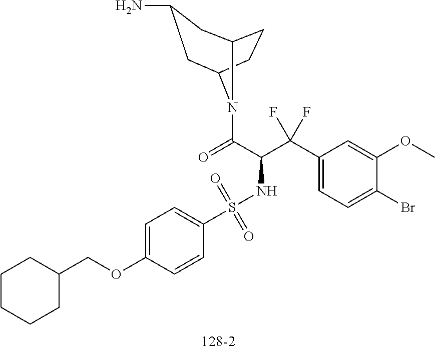 [SMILES: COc1cc(C(F)(F)[C@@H](NS(=O)(=O)c2ccc(OCC3CCCCC3)cc2)C(=O)N2C3CCC2CC(N)C3)ccc1Br]

Compounds 128B, 128C, 128D, 128E, and 128F were synthesized by employing the procedures described for Compound 89B, Compound B6-3, Compounds 42B, 89E, and Compound B2-3 using Compounds 128A, 128B, 128C, 128D, and 128E in lieu of Compound 89A, Compound B6-2, Compounds 42A, 89D, and Compound B2-2.

Compound 128B: ¹H-NMR (CDCl₃, 400 MHz): δ (ppm) 1.32 (d, J=6.4 Hz, 3H), 3.84 (s, 3H), 4.70-4.76 (m, 1H), 5.26 (d, J=4.4 Hz, 1H), 6.84-6.89 (m, 1H), 7.08 (d, J=1.6 Hz, 1H) 7.49 (d, J=8.0 Hz, 1H).

Compound 128C: ¹H-NMR (CDCl₃, 400 MHz): δ (ppm) 2.57 (s, 3H), 3.94 (d, J=6.0 Hz, 3H), 7.36-7.38 (m, 1H), 7.46 (d, J=1.6 Hz, 1H), 7.61 (d, J=8.0 Hz, 1H).

Compound 128D: LC-MS (ESI) m/z: 259 [M+H]⁺.

Compound 128E: ¹H-NMR (CDCl₃, 400 MHz): δ (ppm) 3.97 (s, 3H), 3.99 (s, 3H), 7.50 (d, J=2.0 Hz, 1H), 7.57 (d, J=1.6 Hz, 1H), 7.70 (d, J=8.0 Hz, 1H).

Compound 128F: ¹H-NMR (CDCl₃, 400 MHz): δ (ppm) 3.86 (s, 3H), 3.94 (s, 3H), 7.10-7.06 (m, 2H), 7.63 (d, J=8.4 Hz, 1H).

To a solution of Compound 128F (1 g, 3.4 mmol) in ethanol (20 mL) at −50° C. under nitrogen was added NaBH₄ (129 mg, 3.4 mmol). After stirred at −30° C. for 30 minutes, the reaction mixture was quenched with a hydrochloride solution (1 M, 2 mL) and extracted with ethyl acetate (50 mL×2). The combined organic extracts was washed with brine (20 mL), dried over anhydrous sodium sulfate, filtered, and concentrated to yield Compound 128G.

To a solution of Compound 128G (500 mg, 1.69 mmol) in ethanol (10 mL) was added a solution of KH₂PO₄ (459 mg, 3.38 mmol) and NaCN (196 mg, 3.38 mmol) in water (2 mL). The reaction mixture was stirred at room temperature overnight, diluted with water (20 mL), and extracted with ethyl acetate (50 mL×2). The combined organic extracts were washed with brine (20 mL), dried over anhydrous sodium sulfate, filtered, and concentrated to give a crude product, which was purified by silica gel column chromatography to afford Compound 128H. ¹H-NMR (CDCl₃, 400 MHz): δ (ppm) 3.95 (s, 3H), 4.81-4.83 (m, 1H), 7.04-7.08 (m, 2H), 7.66 (d, J=8.4 Hz, 1H).

Compounds 1281, 128J, 128K, 128L, 128M, 128N, and 123 were synthesized by employing the procedures described for Compound 45G, Compound B6-3, Compounds 451, 80A, Compound B4, Compounds 2D, and 1 using Compounds 128H, 128I, 128J, 128K, 128L, Compound B1, A3, Compounds 128M, and 123N in lieu of Compound 45F, Compound B6-2, Compounds 45H, 51J, Compounds B2-6, B4-1, Compound 2C, Compound B2, and Compound 1A. Compound 1281: LC-MS (ESI) m/z: 347 [M+Na]⁺; ¹H-NMR (CDCl₃, 400 MHz): δ (ppm) 3.29 (d, J=7.6 Hz, 1H), 3.85 (s, 3H), 3.92 (s, 3H), 4.50-4.57 (m, 1H), 6.94-7.00 (m, 2H), 7.59 (d, J=8.4 Hz, 1H).

Compound 128J: ¹H-NMR (CDCl₃, 400 MHz): δ (ppm) 3.92 (s, 3H), 3.96 (s, 3H), 7.05-7.07 (m, 1H), 7.11 (d, J=1.6 Hz, 1H), 7.59 (d, J=8.4 Hz, 1H).

Compound 128K: ¹H-NMR (CD₃OD, 400 MHz): δ (ppm) 3.88 (s, 3H), 7.02-7.04 (m, 1H), 7.15 (s, 1H), 7.55 (d, J=8.0 Hz, 1H).

Compound 128L: ¹H-NMR (DMSO-d₆, 400 MHz): δ (ppm) 3.91 (s, 3H), 5.04 (m, 1H), 7.07 (M, 1H), 7.30 (S, 1H), 7.74 (d, J=8.0 Hz, 1H).

Compound 128M: LC-MS (ESI) m/z: 518 [M+H]⁺.

Compound 128N: LC-MS (ESI) m/z: 670 [M−Boc+H]⁺.

Compound 128 was separated with chiral HPLC to give Compound 128-1 and Compound 128-2.

Compound 128-1: LC-MS (ESI) m/z: 670 [M+H]⁺; ¹H-NMR (CD₃OD, 400 MHz): δ (ppm) 1.11-1.42 (m, 5H), 1.62-2.18 (m, 14H), 3.65-3.90 (m, 6H), 4.63-4.83 (m, 3H), 6.84-7.06 (m, 4H), 7.42-7.58 (m, 3H). Chiral separation condition: MeOH contained 0.2% Methanol Ammonia; OD-H (150×4.6 mm, 5 μm); retention time: 5.29 minute.

Compound 128-2: LC-MS (ESI) m/z: 670 [M+H]⁺; ¹H-NMR (CD₃OD, 400 MHz): δ (ppm) 1.09-1.42 (m, 5H), 1.59-2.19 (m, 14H), 3.64-3.90 (m, 6H), 4.63-4.83 (m, 3H), 6.84-7.05 (m, 4H), 7.42-7.58 (m, 3H). Chiral separation condition: MeOH contained 0.2% Methanol Ammonia; OD-H (150×4.6 mm, 5 μm); retention time: 4.21 minute.

### Example 129

### Synthesis of (R)—N-(3-(4-aminopiperidin-1-yl)-1-(4-chloro-3-methoxyphenyl)-1,1-difluoro-3-oxopropan-2-yl)-4-(cyclohexylmethoxy)benzenesulfonamide (Compound 129-1), and (S)—N-(3-(4-aminopiperidin-1-yl)-1-(4-chloro-3-methoxyphenyl)-1,1-difluoro-3-oxopropan-2-yl)-4-(cyclohexylmethoxy)benzenesulfonamide (Compound 129-2)

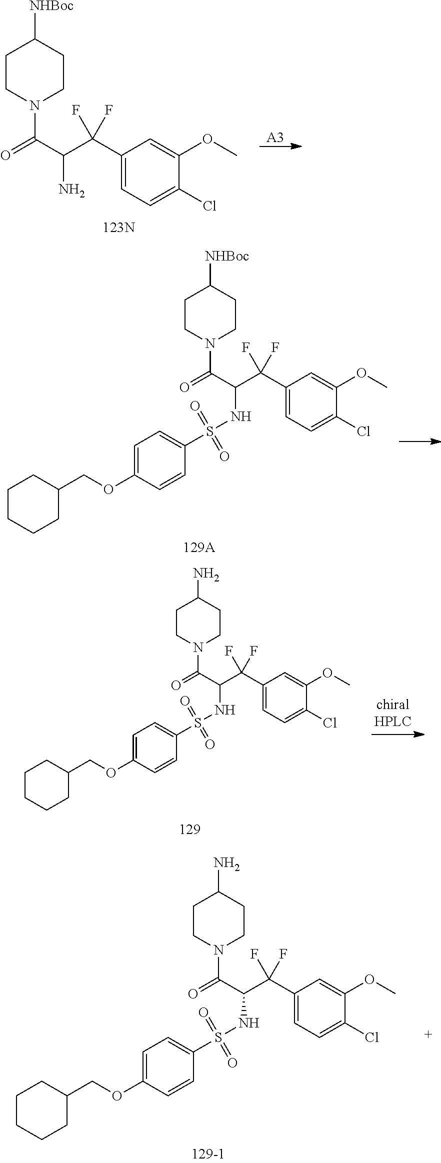

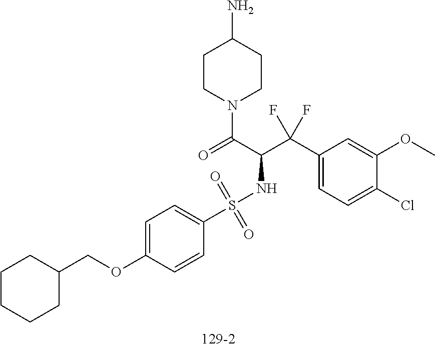 [SMILES: COc1cc(C(F)(F)[C@@H](NS(=O)(=O)c2ccc(OCC3CCCCC3)cc2)C(=O)N2CCC(N)CC2)ccc1Cl]

Compounds 129A and 129 were synthesized by employing the procedures described for Compounds 1A, and 6 using Compound 123N, Compound A3, and Compound 129A in lieu of Compounds B2, A1, and Compound 6C.

Compound 129A: LC-MS (ESI) m/z: 600 [M−Boc+H]⁺.

Compound 129 was separated with chiral HPLC to give Compound 129-1 and Compound 129-2.

Compound 129-1: LC-MS (ESI) m/z: 600 [M+H]⁺; ¹H-NMR (CD₃OD, 400 MHz): δ (ppm) 1.05-1.38 (m, 8H), 1.71-1.90 (m, 8H), 2.41-2.68 (m, 1H), 2.79-2.86 (m, 1H), 2.93-3.02 (m, 1H), 3.84-3.88 (m, 5H), 3.93-4.01 (m, 1H), 4.15-4.31 (m, 1H), 6.91-7.08 (m, 4H), 7.37-7.41 (m, 1H), 7.55-7.58 (m, 2H). Chiral separation condition: MeOH contained 0.2% NH₄OH; OJ-H (4.6×250 mm, 5 μm); retention time: 3.26 minute.

Compound 129-2: LC-MS (ESI) m/z: 600 [M+H]⁺; ¹H-NMR (CD₃OD, 400 MHz): δ (ppm) 0.86-1.38 (m, 8H), 1.71-1.90 (m, 8H), 2.41-2.66 (m, 1H), 2.76-2.83 (m, 1H), 2.92-3.01 (m, 1H), 3.84-3.87 (m, 5H), 3.92-4.00 (m, 1H), 4.14-4.30 (m, 1H), 6.91-7.07 (m, 4H), 7.37-7.41 (m, 1H), 7.55-7.58 (m, 2H). Chiral separation condition: MeOH contained 0.2% NH₄OH; OJ-H (4.6×250 mm, 5 μm); retention time: 2.54 minute.

### Example 130

### Synthesis of N-((2R)-3-(3-amino-8-azabicyclo[3.2.1]octan-8-yl)-1-(5-chlorothiophen-2-yl)-1,1-difluoro-3-oxopropan-2-yl)-6-(cyclopentyloxy)naphthalene-2-sulfonamide (Compound 130-1), and N-((2S)-3-(3-amino-8-azabicyclo[3.2.1]octan-8-yl)-1-(5-chlorothiophen-2-yl)-1,1-difluoro-3-oxopropan-2-yl)-6-(cyclopentyloxy)naphthalene-2-sulfonamide (Compound 130-2)

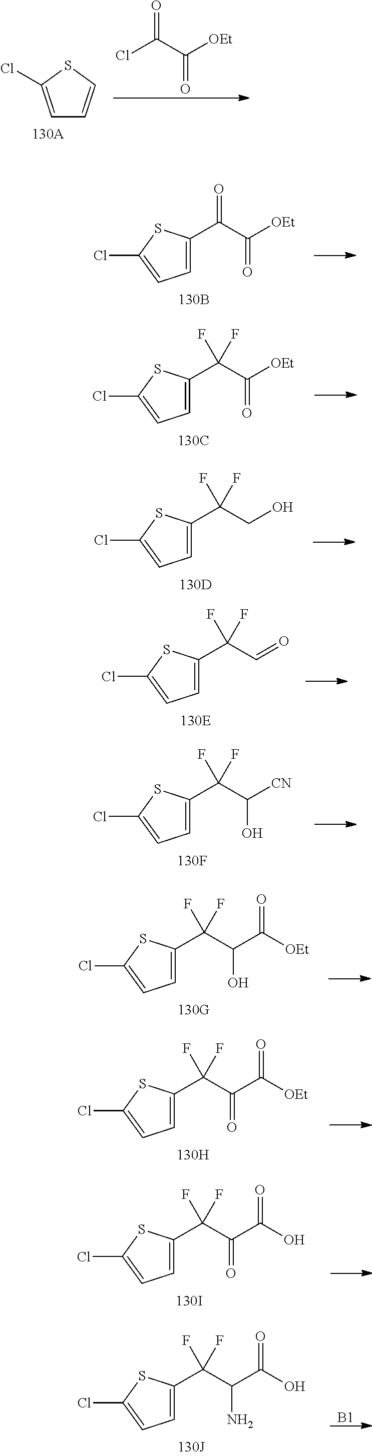

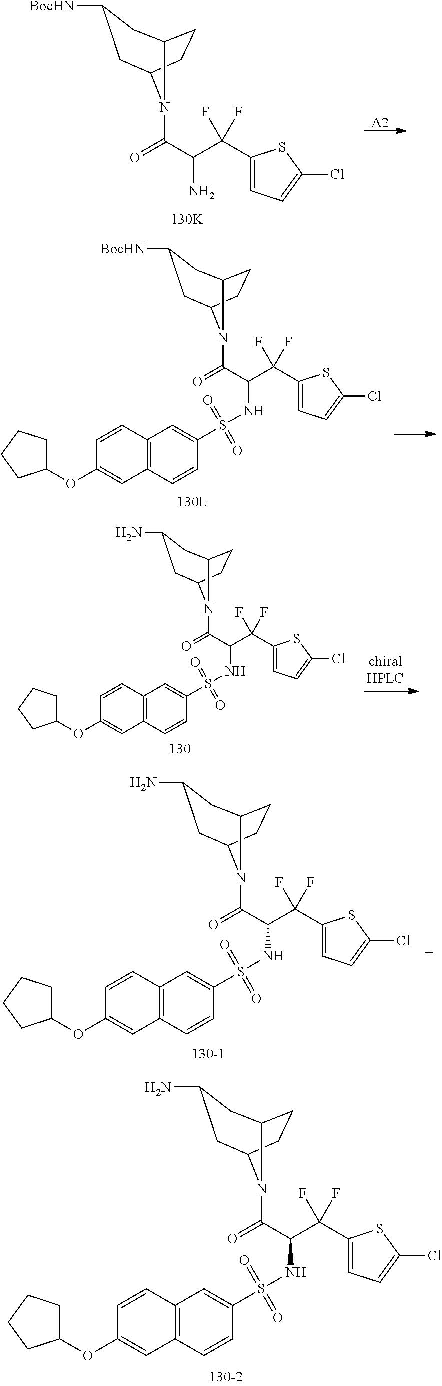 [SMILES: CC(C)(C)OC(=O)NC1CC2CCC(C1)N2C(=O)C(N)C(F)(F)c1ccc(Cl)s1 ; "A2" ; CC(C)(C)OC(=O)NC1CC2CCC(C1)N2C(=O)C(NS(=O)(=O)c1ccc2cc(OC3CCCC3)ccc2c1)C(F)(F)c1ccc(Cl)s1 ; NC1CC2CCC(C1)N2C(=O)C(NS(=O)(=O)c1ccc2cc(OC3CCCC3)ccc2c1)C(F)(F)c1ccc(Cl)s1 ; NC1CC2CCC(C1)N2C(=O)[C@@H](NS(=O)(=O)c1ccc2cc(OC3CCCC3)ccc2c1)C(F)(F)c1ccc(Cl)s1 ; NC1CC2CCC(C1)N2C(=O)[C@H](NS(=O)(=O)c1ccc2cc(OC3CCCC3)ccc2c1)C(F)(F)c1ccc(Cl)s1]

Compounds 130B, 130C, 130D, 130E, 130F, 130G, 130H, 130I, 130J, 130K, 130L, and 130 were synthesized by employing the procedures described for Compounds B2-2, B2-3, Compound 51E, Compound B6-3, Compounds 45F, 45G, Compound B6-3, Compounds 451, 80A, Compound B4, Compounds 1A, and 6 using Compounds 130A, 130B, 130C, 130D, 130E, 130F, 130G, 130H, 130I, 130J, Compounds B1, A2, Compounds 130K, and 130L in lieu of Compounds B2-1, B2-2, Compound 51D, Compound B6-2, Compounds 45E, 45F, Compound B6-2, Compounds 4511, 51J, Compounds B2-6, B4-1, A1, B2, and Compound 6C.

Compound 130B: LC-MS (ESI) m/z: 219[M+H]⁺; ¹H-NMR (CDCl₃, 400 MHz): δ (ppm) 1.43 (t, J=6.8 Hz, 3H), 4.43 (q, J=7.2 Hz, 2H), 7.03-7.04 (m, 1H), 7.97-7.98 (m, 1H).

Compound 130C: ¹H-NMR (CDCl₃, 400 MHz): δ (ppm) 1.37 (t, J=6.8 Hz, 3H), 4.38 (q, J=7.2 Hz, 2H), 6.89-6.90 (m, 1H), 7.18-7.19 (m, 1H).

Compound 130D: LC-MS (ESI) m/z: 179 [M−F]⁺; ¹H-NMR (CDCl₃, 400 MHz): δ (ppm) 2.11-2.15 (brs, 1H), 4.02 (t, J=12.4 Hz, 2H), 6.88-6.89 (m, 1H), 7.09-7.11 (m, 1H).

Compound 130E was used directly for the next step without further purification. LC-MS (ESI) m/z: non-ionizable compound under routine conditions used.

Compound 130F: LC-MS (ESI) m/z: 224[M+H]⁺; ¹H-NMR (CDCl₃, 400 MHz): δ (ppm) δ (ppm) 4.83-4.87 (m, 1H), 6.92-6.95 (m, 1H), 7.22-7.23 (m, 1H).

Compound 130G: LC-MS (ESI) m/z: 293 [M+Na]⁺; ¹H-NMR (CDCl₃, 400 MHz): δ (ppm) 1.32 (t, J=7.6 Hz, 3H), 4.41 (d, J=7.2 Hz, 1H), 4.31-4.34 (m, 2H), 4.51-4.52 (m, 1H), 6.87-6.88 (m, 1H), 7.09-7.10 (m, 1H).

Compound 130H: LC-MS (ESI) m/z: 267[M−H]; ¹H-NMR (CDCl₃, 400 MHz): δ (ppm) 1.35-1.37 (t, J=7.6 Hz, 3H), 4.41-4.43 (m, 2H), 6.86-6.87 (m, 1H), 7.17-7.18 (m, 1H).

Compound 1301: LC-MS (ESI) m/z: 239 [M−H]⁺; ¹H-NMR (CD₃OD, 400 MHz): δ (ppm) 6.83-6.84 (m, 1H), 7.06-7.08 (m, 1H).

Compound 130J: LC-MS (ESI) m/z: 242 [M+H]⁺; ¹H-NMR (CD₃OD, 400 MHz): δ (ppm) 4.47-4.53 (m, 1H), 7.04-7.05 (m, 1H), 7.29-7.31 (m, 1H).

Compound 130K: LC-MS (ESI) m/z: 450 [M+H]⁺.

Compound 130L was used directly in the next step. LC-MS (ESI) m/z: 724 [M+H]⁺; ¹H-NMR (CD₃OD, 400 MHz): δ (ppm) 0.44-1.12 (m, 3H), 1.19-1.36 (m, 9H), 1.46-2.09 (m, 13H), 3.56-3.72 (m, 1H), 4.03-4.74 (m, 2H), 4.83-4.95 (m, 1H), 6.67-6.82 (m, 1H), 7.03-7.22 (m, 3H), 7.58-7.82 (m, 3H), 8.15-8.18 (m, 1H).

Compound 130 was further separated by chiral HPLC to furnish Compound 130-1 and Compound 130-2.

Compound 130-1: LC-MS (ESI) m/z: 624 [M+H]⁺; ¹H-NMR (CD₃OD, 400 MHz): δ (ppm) 0.47-0.84 (m, 2H), 1.36-2.11 (m, 14H), 2.78-3.12 (m, 1H), 4.11-4.74 (m, 3H), 4.89-4.90 (m, 1H), 6.70-6.81 (m, 1H), 7.02-7.22 (m, 3H), 7.58-7.82 (m, 3H), 8.15-8.19 (m, 1H). Chiral separation condition: MeOH contained 0.5% NH₄OH; IC (4.6×250 mm, 5 μm); retention time: 3.85 minute.

Compound 130-2: LC-MS (ESI) m/z: 624 [M+H]⁺; ¹H-NMR (CD₃OD, 400 MHz): δ (ppm) 0.48-0.96 (m, 2H), 1.26-2.14 (m, 14H), 2.78-3.12 (m, 1H), 4.13-4.64 (m, 3H), 4.89-4.91 (m, 1H), 6.71-6.81 (m, 1H), 7.04-7.22 (m, 3H), 7.58-7.82 (m, 3H), 8.15-8.20 (m, 1H). Chiral separation condition: MeOH contained 0.5% NH₄OH; IC (4.6×250 mm, 5 μm); retention time: 3.05 minute.

### Example 131

### Synthesis of (R)—N-(3-(4-aminopiperidin-1-yl)-1-(5-chlorothiophen-2-yl)-1,1-difluoro-3-oxopropan-2-yl)-6-(cyclopentyloxy)naphthalene-2-sulfonamide (Compound 131-1), and (S)—N-(3-(4-aminopiperidin-1-yl)-1-(5-chlorothiophen-2-yl)-1,1-difluoro-3-oxopropan-2-yl)-6-(cyclopentyloxy)naphthalene-2-sulfonamide (Compound 131-2)

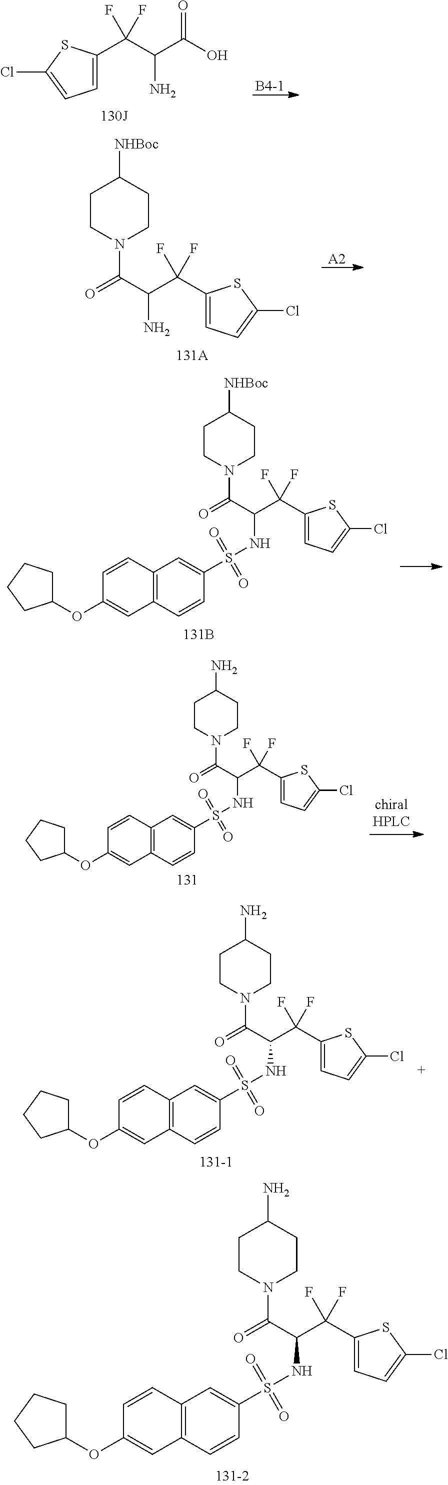

Compounds 131A, 131B, and 131 were synthesized by employing the procedures described for Compound B4, Compounds 1A, and 6 using Compound 130J, Compound A2, Compounds 131A, and 131B in lieu of Compounds B2-6, A1, B2, and Compound 6C.

Compound 131A: LC-MS (ESI) m/z: 424 [M+H]⁺.

Compound 131B was used directly for the next step. LC-MS (ESI) m/z: 698 [M+H]⁺.

Compound 131 was separated by chiral HPLC to furnish Compound 131-1 and Compound 131-2.

Compound 131-1: LC-MS (ESI) m/z: 598 [M+H]⁺; ¹H-NMR (CD₃OD, 400 MHz): δ (ppm) 0.41-1.05 (m, 2H), 1.56-1.85 (m, 8H), 1.94-1.25 (m, 3H), 2.65-2.95 (m, 2H), 3.83-4.13 (m, 2H), 4.89-4.94 (m, 2H), 6.66-6.79 (m, 1H), 7.02-7.23 (m, 3H), 7.56-7.60 (m, 1H), 7.74-7.82 (m, 2H), 8.15-8.16 (m, 1H). Chiral separation condition: MeOH contained 0.2% NH₃-MeOH; OJ (4.6×250 mm, 5 μm); retention time: 3.80 minute.

Compound 131-2: LC-MS (ESI) m/z: 598 [M+H]⁺; ¹H-NMR (CD₃OD, 400 MHz): δ (ppm) 0.61-0.98 (m, 2H), 1.46-1.85 (m, 8H), 1.94-1.24 (m, 3H), 2.56-2.85 (m, 2H), 3.73-4.13 (m, 2H), 4.83-4.94 (m, 2H), 6.69-6.78 (m, 1H), 7.12-7.26 (m, 3H), 7.57-7.61 (m, 1H), 7.73-7.83 (m, 2H), 8.15-8.17 (m, 1H). Chiral separation condition: MeOH contained 0.2% NH₃-MeOH; OJ (4.6×250 mm, 5 μm); retention time: 2.89 minute.

### Example 132

### Synthesis of N—((R)-3-((S)-3-aminopiperidin-1-yl)-1-(4-bromophenyl)-1,1-difluoro-3-oxopropan-2-yl)-6-(cyclopentyloxy)naphthalene-2-sulfonamide trifluoroacetate (Compound 132-1), and N—((S)-3-((S)-3-aminopiperidin-1-yl)-1-(4-bromophenyl)-1,1-difluoro-3-oxopropan-2-yl)-6-(cyclopentyloxy)naphthalene-2-sulfonamide trifluoroacetate (Compound 132-2)

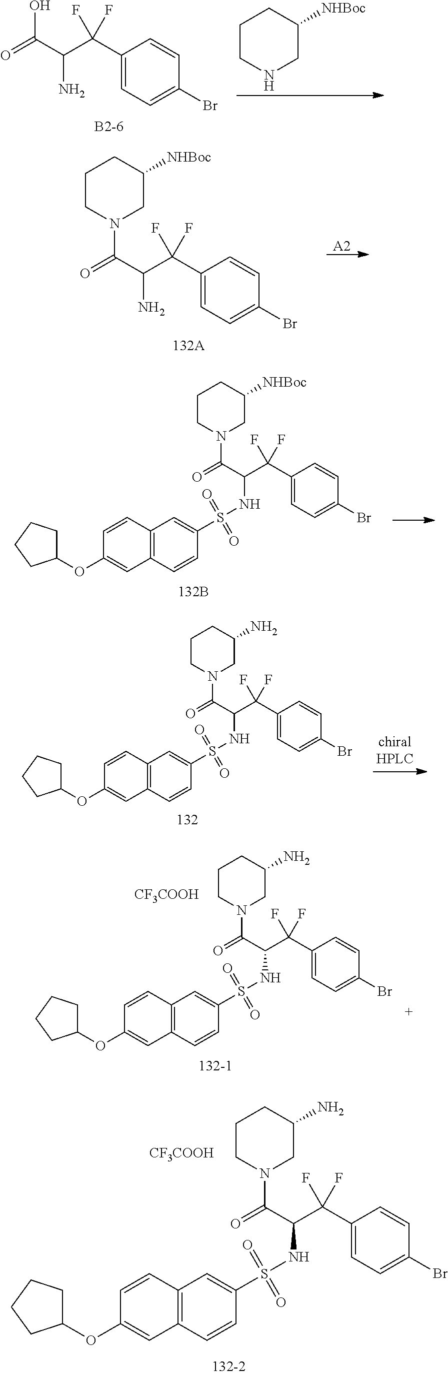

Compounds 132A, 132B, and 132 were synthesized by employing the procedures described for Compound B4, Compounds 2D, and 1 using (S)-tert-butyl piperidin-3-ylcarbamate, Compound 132A, Compound A2, and Compound 132B in lieu of Compounds B4-1, B2, Compounds 2C, and 1A.

Compound 132A: LC-MS (ESI) m/z: 462 [M+H]⁺.

Compound 132B: LC-MS (ESI) m/z: 636 [M−Boc+H]⁺.

Compound 132 was separated with chiral HPLC to afford Compound 132-1 and Compound 132-2.

Compound 132-1: LC-MS (ESI) m/z: 636 [M+H]⁺. ¹H-NMR (MeOD, 400 MHz): δ (ppm) 1.33-3.00 (m, 16H), 3.94-4.28 (m, 2H), 5.00-5.02 (m, 1H), 7.24-7.35 (m, 4H), 7.38-7.55 (m, 3H), 7.76-7.78 (m, 1H), 7.87 (d, J=8.8 Hz, 1H), 8.16-8.18 (m, 1H). Chiral separation condition: n-Hexane/EtOH contained 0.1% DEA, 60/40; IC (250×4.6 mm, 5 μm); retention time: 10.79 minute.

Compound 132-2: LC-MS (ESI) m/z: 636 [M+H]⁺. ¹H-NMR (MeOD, 400 MHz): δ (ppm) 1.26-2.12 (m, 13H), 2.36-3.00 (m, 3H), 3.99-4.51 (m, 2H), 4.95-5.04 (m, 1H), 7.26 (d, J=6.4 Hz, 1H), 7.31-7.34 (m, 3H), 7.40-7.45 (m, 2H), 7.54-7.56 (m, 1H), 7.77 (d, J=8.8 Hz, 1H), 7.87 (d, J=8.8 Hz, 1H), 8.13-8.16 (m, 1H). Chiral separation condition: n-Hexane/EtOH contained 0.1% DEA, 60/40; IC (250×4.6 mm, 5 μm); retention time: 8.77 minute.

### Example 133

### Synthesis of N-((2R)-3-(3-amino-8-azabicyclo[3.2.1]octan-8-yl)-1-(4′-chloro-[1,1′-biphenyl]-4-yl)-1,1-difluoro-3-oxopropan-2-yl)-2-(6-(cyclopentyloxy)naphthalen-2-yl)-2-oxoacetamide trifluoroacetate (Compound 133-1), and N-((2S)-3-(3-amino-8-azabicyclo[3.2.1]octan-8-yl)-1-(4′-chloro-[1,1′-biphenyl]-4-yl)-1,1-difluoro-3-oxopropan-2-yl)-2-(6-(cyclopentyloxy)naphthalen-2-yl)-2-oxoacetamide trifluoroacetate (Compound 133-2)

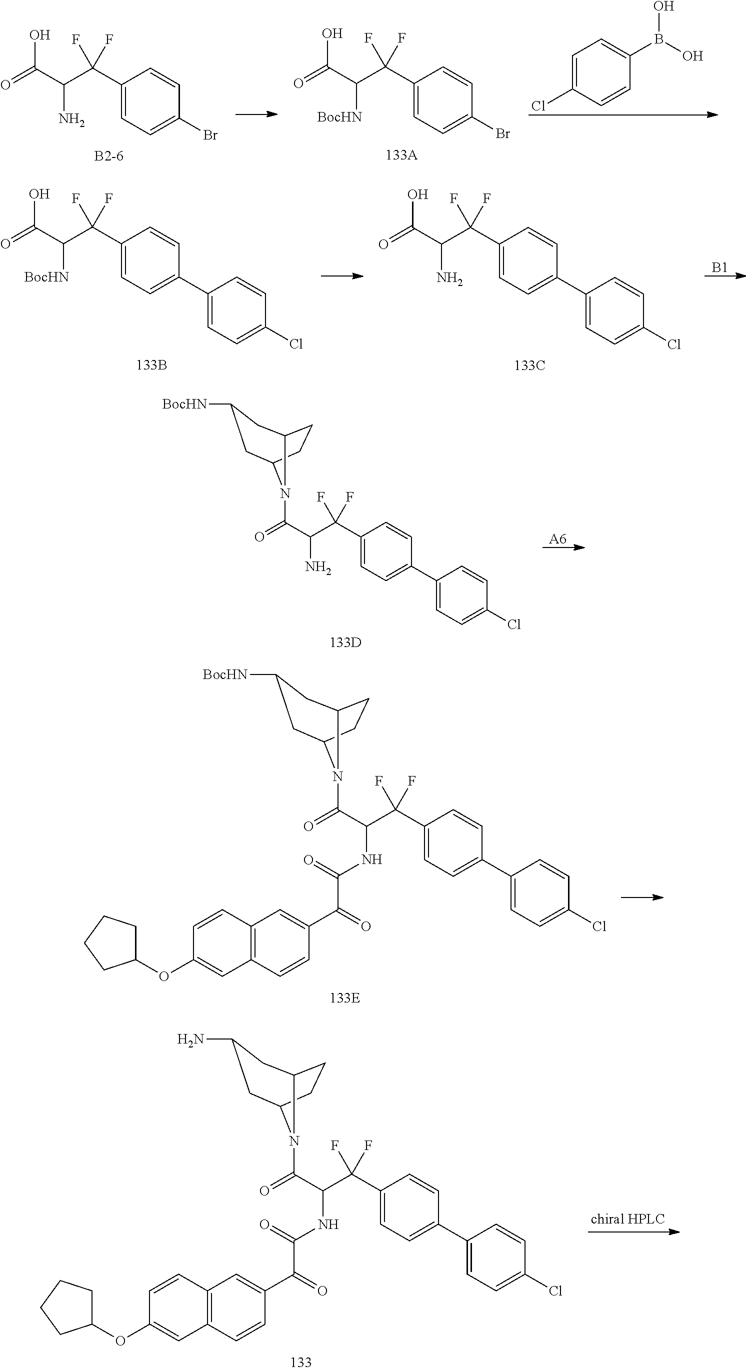

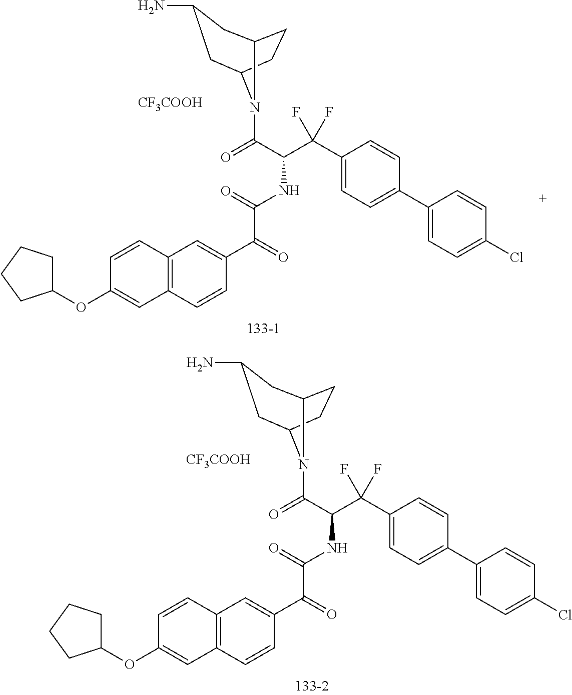 [SMILES: NC1CC2CCC(C1)N2C(=O)[C@@H](NC(=O)C(=O)c1ccc2cc(OC3CCCC3)ccc2c1)C(F)(F)c1ccc(-c2ccc(Cl)cc2)cc1 ; "CF3COOH" ; NC1CC2CCC(C1)N2C(=O)[C@H](NC(=O)C(=O)c1ccc2cc(OC3CCCC3)ccc2c1)C(F)(F)c1ccc(-c2ccc(Cl)cc2)cc1 ; "CF3COOH"]

To a suspension of Compound B2-6 (7.1 g, 22.5 mmol) in MeCN (300 mL) was added triethylamine (6.81 g, 67.5 mmol) and stirred at room temperature until the mixture was turned clear (approx. 30 minutes). To the solution was added di-tert-butyl dicarbonate (5.38 g, 24.7 mmol) and stir at room temperature overnight. The reaction mixture was concentrated under reduced pressure. The residue was diluted with water (200 mL) and washed with diethyl ether (100 mL×2). The aqueous phase was acidified to pH 3 with 10% HCl and extracted with ethyl acetate (100 mL×2). The combined organic extracts were washed with brine (100 mL), dried over magnesium sulphate, filtered, and concentrated to yield Compound 133A. LC-MS (ESI) m/z: 380 [M+H]⁺.

Compounds 133B, 133C, 133D, 133E, and 133 were synthesized by employing the procedures described for Compounds 14C, 1, Compounds B4, B4, and Compounds 1 using 4-chlorophenylboronic acid, 133A using Na₂CO₃ as base, 133B, 133C, Compounds B1, A6, Compounds 133D using DMF as solvent and without DIPEA, and 133E in lieu of Compounds 14B, 14A using K₂CO₃ as base, 1A, Compounds B2-6, B4-1, B2-6, B4-1 using dichloromethane as solvent and with DIPEA, and Compound 1A.

Compound 133B: LC-MS (ESI) m/z: 412 [M+H]⁺; ¹H-NMR (CD₃OD, 400 MHz): δ (ppm) 1.37 (s, 9H), 4.49-4.82 (m, 1H), 7.33-7.35 (m, 2H), 7.47-7.56 (m, 6H).

Compound 133C: LC-MS (ESI) m/z: 312 [M+H]⁺.

Compound 133D: LC-MS: (m/z) 520 [M+1]⁺.

Compound 133E: LC-MS: (m/z) 786 [M+1]⁺.

Compound 133 was separated with chiral HPLC to give Compound 133-1 and Compound 133-2.

Compound 133-1: LC-MS (ESI) m/z: 686 [M+H]⁺; ¹H-NMR (Acetone-d₆, 400 MHz): δ (ppm) 1.69-1.89 (m, 15H), 2.06-2.07 (m, 1H), 2.40-2.57 (m, 1H), 4.67-4.87 (m, 3H), 5.02-5.04 (m, 1H), 5.74-5.75 (m, 1H), 7.09-7.12 (m, 1H), 7.31-7.33 (m, 1H), 7.51-7.53 (m, 2H), 7.73-7.88 (m, 8H), 7.96-7.99 (m, 1H), 8.49-8.65 (m, 1H). Chiral separation condition: n-Hexane/EtOH contained 0.1% DEA, 50/50; OZ-H (4.6×250 mm, 5 μm); retention time: 17.36 minute.

Compound 133-2: LC-MS (ESI) m/z: 686 [M+H]⁺; ¹H-NMR (Acetone-d₆, 400 MHz): δ (ppm) 1.80-1.96 (m, 15H), 2.07-2.09 (m, 1H), 2.56-2.88 (m, 1H), 4.61-4.67 (m, 3H), 5.02-5.04 (m, 1H), 5.73-5.74 (m, 1H), 7.10-7.13 (m, 1H), 7.33-7.35 (m, 1H), 7.52-7.55 (m, 2H), 7.73-7.88 (m, 8H), 7.97-8.00 (m, 1H), 8.62-8.64 (m, 1H). Chiral separation condition: n-Hexane/EtOH contained 0.1% DEA, 50/50; OZ-H (4.6×250 mm, 5 μm); retention time: 8.29 minute.

### Example 134

### Synthesis of (R)—N-(3-(4-aminopiperidin-1-yl)-1-(4′-chloro-[1,1′-biphenyl]-4-yl)-1,1-difluoro-3-oxopropan-2-yl)-2-(4-(cyclohexylmethoxy)phenyl)-2-oxoacetamide trifluoroacetate (Compound 134-1), and (S)—N-(3-(4-aminopiperidin-1-yl)-1-(4′-chloro-[1,1′-biphenyl]-4-yl)-1,1-difluoro-3-oxopropan-2-yl)-2-(4-(cyclohexylmethoxy)phenyl)-2-oxoacetamide trifluoroacetate (Compound 134-2)

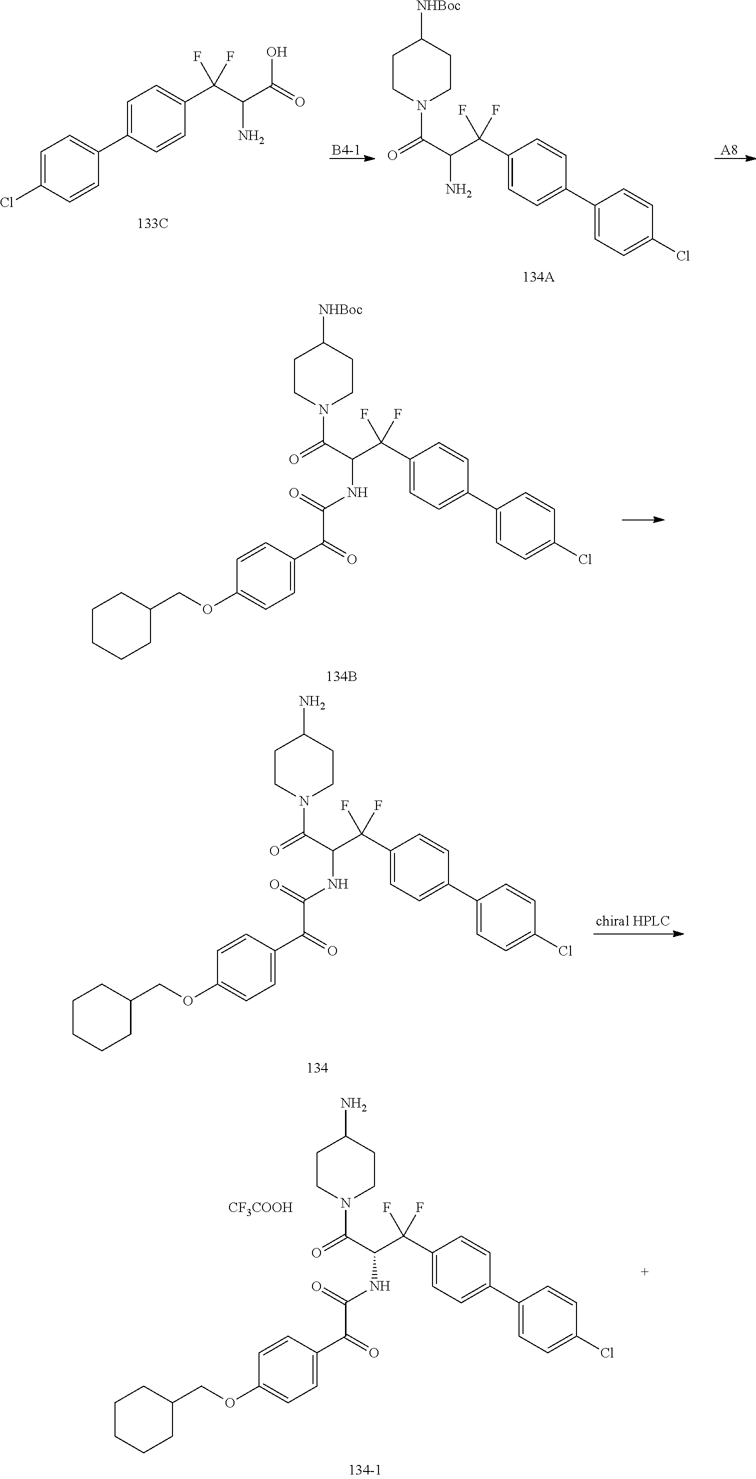

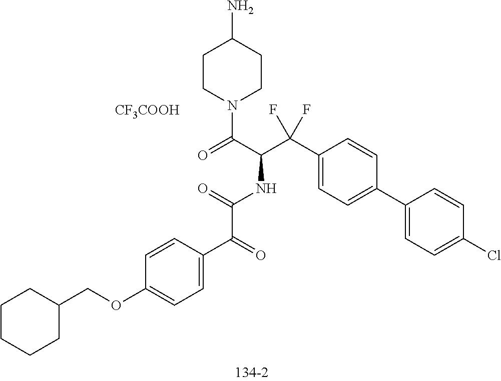 [SMILES: NC1CCN(C(=O)[C@H](NC(=O)C(=O)c2ccc(OCC3CCCCC3)cc2)C(F)(F)c2ccc(-c3ccc(Cl)cc3)cc2)CC1 ; "CF3COOH"]

Compounds 134A, 134B, and 134 were synthesized by employing the procedures described for Compounds B4, B4, and Compound 1 using Compound 133C, Compound A8, Compounds 134A using DMF as solvent and without DIPEA, and 134B in lieu of Compounds B2-6, B2-6, B4-1 using dichloromethane as solvent and with DIPEA, and Compound 1A.

Compound 134A: LC-MS (ESI) m/z: 494 [M+H]⁺.

Compound 134B: LC-MS (ESI) m/z: 682 [M−55]⁺.

Compound 134 was separated with chiral HPLC to give Compound 134-1 and Compound 134-2.

Compound 134-1: LC-MS (ESI) m/z: 638 [M+H]⁺; ¹H-NMR (Actone-d₆, 400 MHz): δ (ppm) 1.05-1.33 (m, 6H), 1.70-1.91 (m, 9H), 2.30-2.56 (m, 2H), 3.74-4.60 (m, 5H), 5.84-6.01 (m, 1H), 6.83-6.97 (m, 2H), 7.52-7.87 (m, 10H). Chiral separation condition: n-Hexane/EtOH contained 0.1% DEA, 50/50; AY-H (150×4.6 mm, 5 μm); retention time: 8.78 minute.

Compound 134-2: LC-MS (ESI) m/z: 638 [M+H]⁺; ¹H-NMR (Actone-d₆, 400 MHz): δ (ppm) 0.91-1.11 (m, 7H), 1.69-1.92 (m, 8H), 2.21-2.54 (m, 2H), 3.62-4.57 (m, 5H), 5.83-5.86 (m, 1H), 6.71-6.80 (m, 2H), 7.42-7.44 (m, 2H), 7.64-7.73 (m, 8H). Chiral separation condition: n-Hexane/EtOH contained 0.1% DEA, 50/50; AY-H (150×4.6 mm, 5 μm); retention time: 13.52 minute.

### Example 135

### Synthesis of N-((2R)-3-(3-amino-8-azabicyclo[3.2.1]octan-8-yl)-1-(4′-chloro-2-methoxy-[1,1′-biphenyl]-4-yl)-1,1-difluoro-3-oxopropan-2-yl)-6-(cyclopentyloxy) naphthalene-2-sulfonamide trifluoroacetate (Compound 135-1), and N-((2S)-3-(3-amino-8-azabicyclo[3.2.1]octan-8-yl)-1-(4′-chloro-2-methoxy-[1,1′-biphenyl]-4-yl)-1,1-difluoro-3-oxopropan-2-yl)-6-(cyclopentyloxy)naphthalene-2-sulfonamide trifluoroacetate (Compound 135-2)

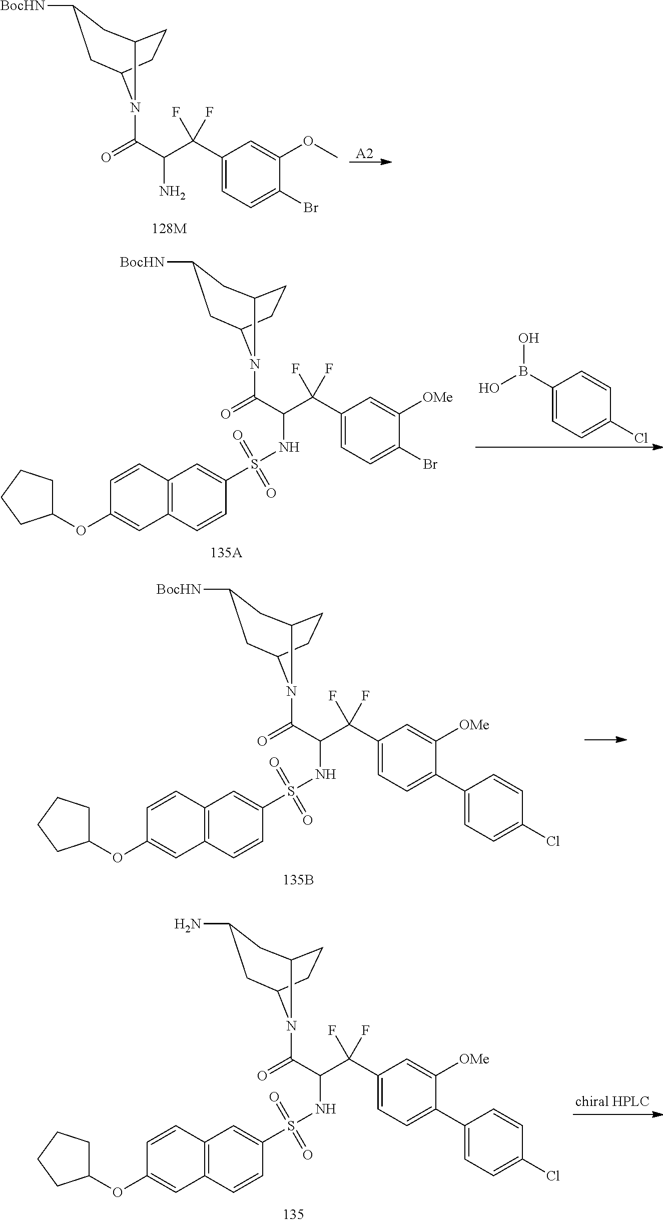

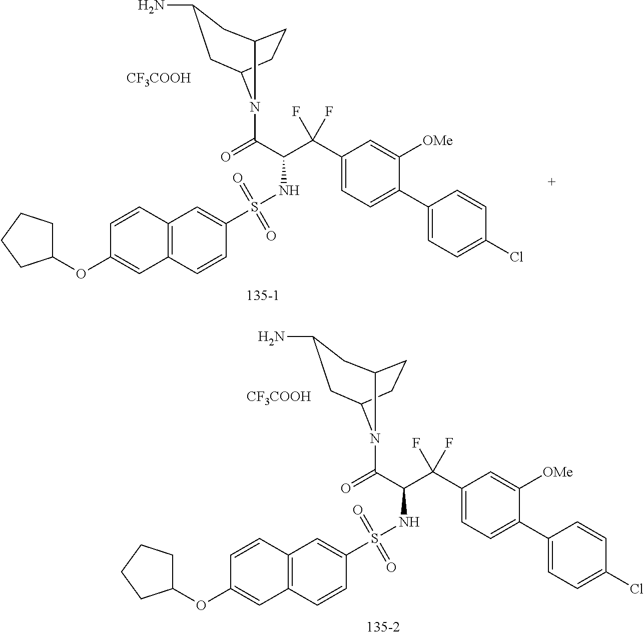 [SMILES: COc1cc(C(F)(F)[C@H](NS(=O)(=O)c2ccc3cc(OC4CCCC4)ccc3c2)C(=O)N2C3CCC2CC(N)C3)ccc1-c1ccc(Cl)cc1 ; "CF3COOH" ; COc1cc(C(F)(F)[C@@H](NS(=O)(=O)c2ccc3cc(OC4CCCC4)ccc3c2)C(=O)N2C3CCC2CC(N)C3)ccc1-c1ccc(Cl)cc1 ; "CF3COOH"]

Compounds 135A, 135B, and 135 were synthesized by employing the procedures described for Compounds 2D, 14C, and 1 using Compound A2, Compounds 128M, 135A, 4-chlorophenylboronic acid, and 135B in lieu of Compound 2C, Compound B2, Compounds 14A, 14B, and 1A.

Compound 135A: LC-MS (ESI) m/z: 692 [M−Boc+H]⁺.

Compound 135B: LC-MS: (ESI) m/z: no mass.

Compound 135 was separated with chiral HPLC to give Compound 135-1 and Compound 135-2.

Compound 135-1: LC-MS (ESI) m/z: 724 [M+H]⁺; ¹H-NMR (CD₃OD, 400 MHz): δ (ppm) 1.60-2.23 (m, 16H), 3.57-3.73 (m, 4H), 4.69-4.83 (m, 3H), 4.91-4.98 (m, 1H), 6.88-7.00 (m, 2H), 7.10-7.25 (m, 4H), 7.35-7.54 (m, 4H), 7.63-7.85 (m, 2H), 8.11-8.16 (m, 1H). Chiral separation condition: MeOH contained 0.2% Methanol Ammonia; (R,R)-Whelk-O1 (150×4.6 mm, 5 μm); retention time: 7.09 minute.

Compound 135-2: LC-MS (ESI) m/z: 724 [M+H]⁺; ¹H-NMR (CD₃OD, 400 MHz): δ (ppm) 1.71-2.23 (m, 16H), 3.57-3.79 (m, 4H), 4.71-4.85 (m, 3H), 4.93-4.98 (m, 1H), 6.87-7.00 (m, 2H), 7.10-7.25 (m, 4H), 7.40-7.54 (m, 4H), 7.63-7.85 (m, 2H), 8.12-8.17 (m, 1H). Chiral separation condition: MeOH contained 0.2% Methanol Ammonia; (R,R)-Whelk-O1 (150×4.6 mm, 5 μm); retention time: 5.78 minute.

### Example 136

### Synthesis of (2R)—N-((2R)-3-(3-amino-8-azabicyclo[3.2.1]octan-8-yl)-1-(4′-chloro-[1,1′-biphenyl]-4-yl)-1,1-difluoro-3-oxopropan-2-yl)-2-(6-(cyclopentyloxy) naphthalen-2-yl)-2-hydroxyacetamide trifluoroacetate (Compound 136-1), (2S)—N-((2S)-3-(3-amino-8-azabicyclo[3.2.1]octan-8-yl)-1-(4′-chloro-[1,1′-biphenyl]-4-yl)-1,1-difluoro-3-oxopropan-2-yl)-2-(6-(cyclopentyloxy)naphthalen-2-yl)-2-hydroxyacetamide trifluoroacetate (Compound 136-2), (2S)—N-((2R)-3-(3-amino-8-azabicyclo[3.2.1]octan-8-yl)-1-(4′-chloro-[1,1′-biphenyl]-4-yl)-1,1-difluoro-3-oxopropan-2-yl)-2-(6-(cyclopentyloxy) naphthalen-2-yl)-2-hydroxyacetamide trifluoroacetate (Compound 136-3), and (2R)—N-((2S)-3-(3-amino-8-azabicyclo[3.2.1]octan-8-yl)-1-(4′-chloro-[1,1′-biphenyl]-4-yl)-1,1-difluoro-3-oxopropan-2-yl)-2-(6-(cyclopentyloxy)naphthalen-2-yl)-2-hydroxyacetamide trifluoroacetate (Compound 136-4)

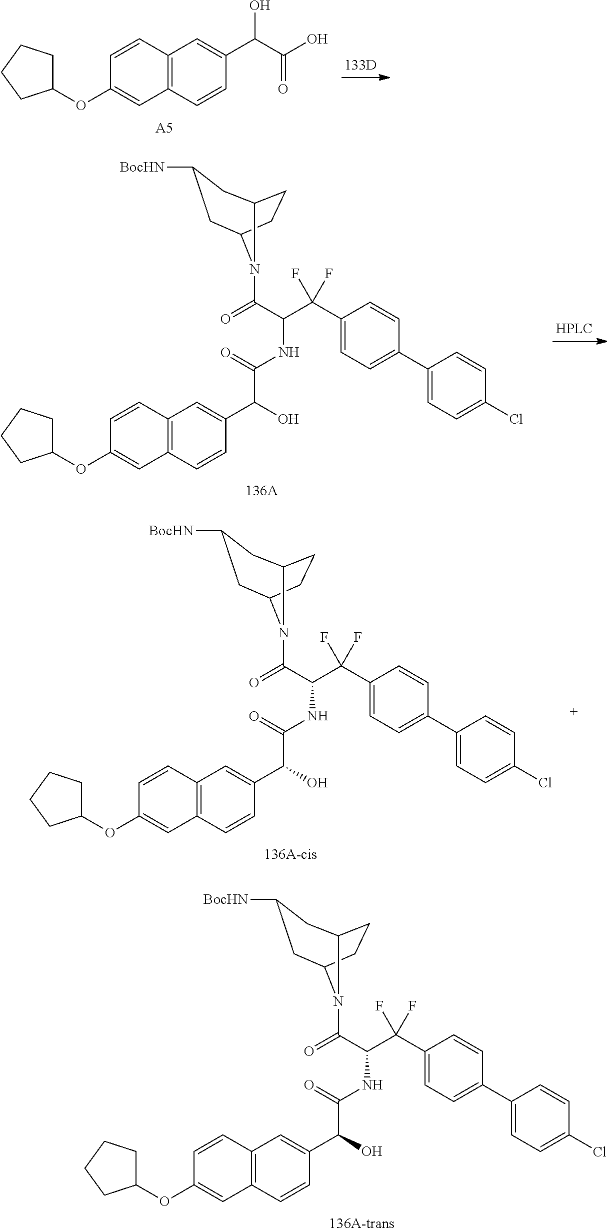

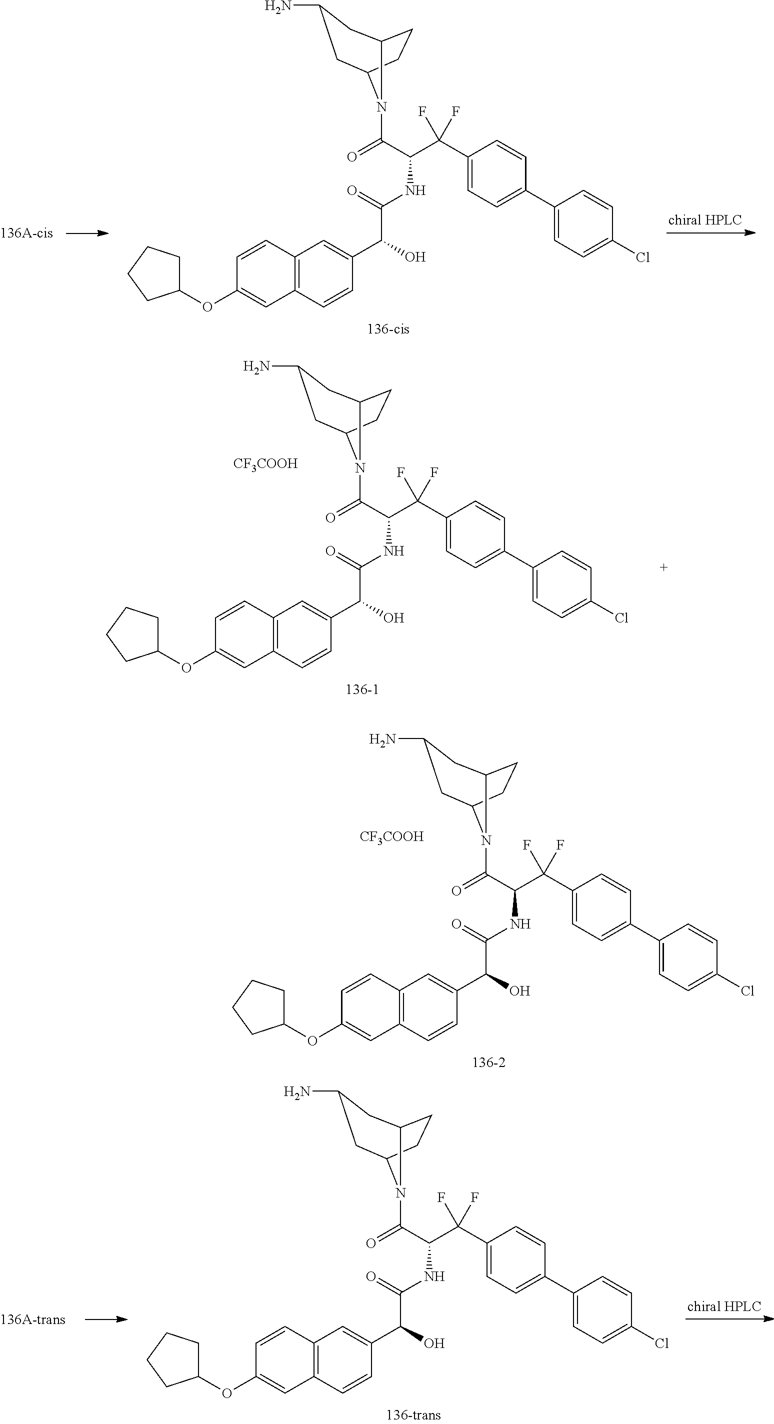 [SMILES: NC1CC2CCC(C1)N2C(=O)[C@@H](NC(=O)[C@H](O)c1ccc2cc(OC3CCCC3)ccc2c1)C(F)(F)c1ccc(-c2ccc(Cl)cc2)cc1 ; NC1CC2CCC(C1)N2C(=O)[C@@H](NC(=O)[C@H](O)c1ccc2cc(OC3CCCC3)ccc2c1)C(F)(F)c1ccc(-c2ccc(Cl)cc2)cc1 ; "CF3COOH" ; NC1CC2CCC(C1)N2C(=O)[C@H](NC(=O)[C@@H](O)c1ccc2cc(OC3CCCC3)ccc2c1)C(F)(F)c1ccc(-c2ccc(Cl)cc2)cc1 ; "CF3COOH" ; NC1CC2CCC(C1)N2C(=O)[C@@H](NC(=O)[C@@H](O)c1ccc2cc(OC3CCCC3)ccc2c1)C(F)(F)c1ccc(-c2ccc(Cl)cc2)cc1]

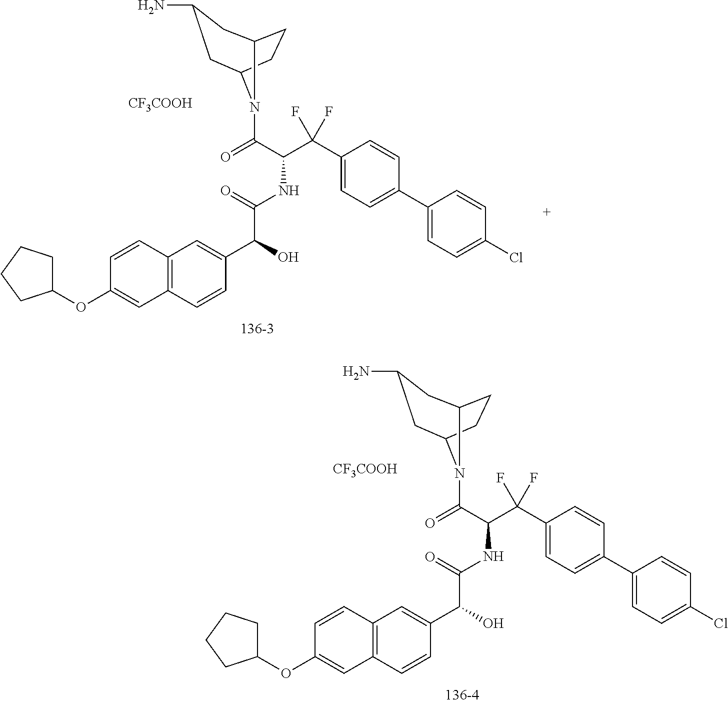 [SMILES: NC1CC2CCC(C1)N2C(=O)[C@@H](NC(=O)[C@@H](O)c1ccc2cc(OC3CCCC3)ccc2c1)C(F)(F)c1ccc(-c2ccc(Cl)cc2)cc1 ; "CF3COOH" ; NC1CC2CCC(C1)N2C(=O)[C@H](NC(=O)[C@H](O)c1ccc2cc(OC3CCCC3)ccc2c1)C(F)(F)c1ccc(-c2ccc(Cl)cc2)cc1 ; "CF3COOH"]

Compound 136A was synthesized by employing the procedure described for Compound B4 using Compound A5 and Compound 133D in lieu of Compound B2-6 and B4-1, which was separated with HPLC to give Compound 136A-cis (yield 14.3%), LC-MS (ESI) m/z: 788 [M+H]⁺, and Compound 136A-trans (yield 9.3%), LC-MS (ESI) m/z: 788 [M+H]⁺.

Compounds 136-cis and 136-trans were respectely synthesized by employing the procedure described for Compound 6 using Compounds 136A-cis and 136A-trans in lieu of Compound 6C.

Compound 136-cis was separated with chiral HPLC to give Compound 136-1 and Compound 136-2.

Compound 136-1: LC-MS (ESI) m/z: 688 [M+H]⁺; ¹H-NMR (Acetone-d₆, 400 MHz): δ (ppm) 1.66-1.91 (m, 15H), 2.17-2.24 (m, 2H), 3.85-4.66 (m, 3H), 4.77-4.78 (m, 1H), 5.30-5.60 (m, 2H), 7.09-7.18 (m, 2H), 7.23-7.52 (m, 8H), 7.54-7.60 (m, 1H), 7.68-7.78 (m, 2H), 7.85-7.95 (m, 1H). Chiral separation condition: n-Hexane/EtOH contained 0.1% DEA, 40/60; AD-H (4.6×250 mm, 5 μm); retention time: 6.92 minute.

Compound 136-2: LC-MS (ESI) m/z: 688 [M+H]⁺; ¹H-NMR (Acetone-d₆, 400 MHz): δ (ppm) 1.66-1.97 (m, 15H), 2.10-2.26 (m, 2H), 3.87-4.77 (m, 3H), 4.92-4.96 (m, 1H), 5.30-5.65 (m, 2H), 7.08-7.16 (m, 2H), 7.22-7.50 (m, 8H), 7.57-7.62 (m, 1H), 7.66-7.78 (m, 2H), 7.85-7.97 (m, 1H). Chiral separation condition: n-Hexane/EtOH contained 0.1% DEA, 40/60; AD-H (4.6×250 mm, 5 μm); retention time: 9.10 minute.

Compound 136-trans was separated with chiral HPLC to give Compound 136-3 and Compound 136-4.

Compound 136-3: LC-MS (ESI) m/z: 688 [M+H]⁺; ¹H-NMR (Acetone-d₆, 400 MHz): δ (ppm) 1.69-1.99 (m, 15H), 2.08-2.24 (m, 1H), 2.54-2.62 (m, 1H), 3.85-4.73 (m, 3H), 4.96-4.97 (m, 1H), 5.20 (s, 1H), 5.49-5.54 (m, 1H), 7.09-7.11 (m, 1H), 7.22-7.23 (m, 1H), 7.30-7.46 (m, 9H), 7.70-7.83 (m, 3H). Chiral separation condition: MeOH contained 0.5% NH₄OH; IC (4.6×250 mm, 5 μm); retention time: 3.92 minute.

Compound 136-4: LC-MS (ESI) m/z: 688 [M+H]⁺; ¹H-NMR (Acetone-d₆, 400 MHz): δ (ppm) 1.67-1.97 (m, 15H), 2.07-2.23 (m, 2H), 3.90-4.70 (m, 3H), 4.96-4.97 (m, 1H), 5.18 (s, 1H), 5.47-5.54 (m, 1H), 7.09-7.23 (m, 2H), 7.29-7.53 (m, 9H), 7.70-7.84 (m, 3H). Chiral separation condition: MeOH contained 0.5% NH₄OH; IC (4.6×250 mm, 5 μm); retention time: 5.39 minute.

### Example 137

### Synthesis of (R)—N-(1-(4′-chloro-[1,1′-biphenyl]-4-yl)-1,1-difluoro-3-oxo-3-(piperazin-1-yl)propan-2-yl)-6-(cyclopentyloxy)naphthalene-2-sulfonamide trifluoroacetate (Compound 137-1), and (S)—N-(1-(4′-chloro-[1,1′-biphenyl]-4-yl)-1,1-difluoro-3-oxo-3-(piperazin-1-yl)propan-2-yl)-6-(cyclopentyloxy)naphthalene-2-sulfonamide trifluoroacetate (Compound 137-2)

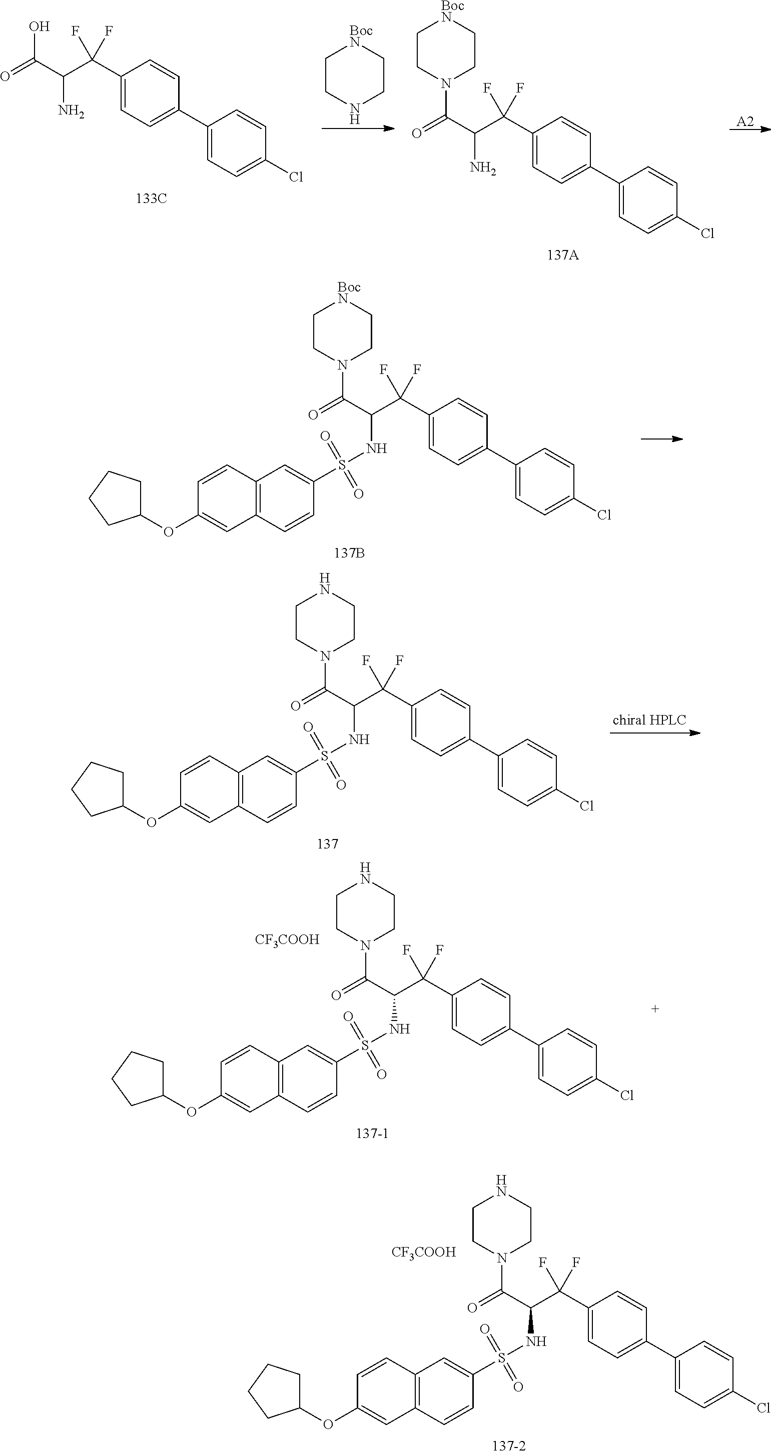

Compounds 137A, 137B, and 137 were synthesized by employing the procedures described for Compound B4, Compounds 2D, and 6 using tert-butyl piperazine-1-carboxylate, Compounds 133C, 137A, Compound A2, and Compound 137B in lieu of Compounds B4-1, B2-6, B2, Compounds 2C, and 6C.

Compound 137A: LC-MS (ESI) m/z: 480 [M+H]⁺.

Compound 137B: LC-MS (ESI) m/z: 754 [M+H]⁺.

Compound 137 was separated with chiral HPLC to furnish Compound 137-1 and Compound 137-2.

Compound 137-1: LC-MS (ESI) m/z: 654 [M+H]⁺; ¹H-NMR (CDCl₃, 400 MHz): δ (ppm) 1.55-1.72 (m, 4H), 1.91-1.97 (m, 4H), 2.86-2.92 (m, 1H), 3.08-3.17 (m, 3H), 3.57-3.63 (m, 1H), 3.79-3.87 (m, 3H), 4.75-4.89 (m, 2H), 6.09-6.18 (m, 1H), 6.92 (s, 1H), 7.09-7.11 (m, 1H), 7.30-7.68 (m, 11H), 8.07 (s, 1H). Chiral separation condition: n-Hexane/EtOH contained 0.1% DEA, 50/50; S,S-Whelk-O1 (4.6×250 mm, 5 μm); 13.71 minute.

Compound 137-2: LC-MS (ESI) m/z: 654 [M+H]⁺; ¹H-NMR (CDCl₃, 400 MHz): δ (ppm) 1.62-1.70 (m, 4H), 1.91-1.97 (m, 4H), 2.87-2.92 (m, 1H), 3.08-3.17 (m, 3H), 3.57-3.63 (m, 1H), 3.79-3.87 (m, 3H), 4.75-4.89 (m, 2H), 6.09-6.18 (m, 1H), 6.92 (s, 1H), 7.09-7.11 (m, 1H), 7.30-7.68 (m, 11H), 8.06 (s, 1H). Chiral separation condition: n-Hexane/EtOH contained 0.1% DEA, 50/50; S,S-Whelk-O1 (4.6×250 mm, 5 μm); retention time: 10.05 minute.

### Example 138

### Synthesis of (R)—N-(3-(4-(aminomethyl)piperidin-1-yl)-1-(4′-chloro-[1,1′-biphenyl]-4-yl)-1,1-difluoro-3-oxopropan-2-yl)-6-(cyclopentyloxy)naphthalene-2-sulfonamide trifluoroacetate (Compound 138-1), and (S)—N-(3-(4-(aminomethyl)piperidin-1-yl)-1-(4′-chloro-[1,1′-biphenyl]-4-yl)-1,1-difluoro-3-oxopropan-2-yl)-6-(cyclopentyloxy)naphthalene-2-sulfonamide trifluoroacetate (Compound 138-2)

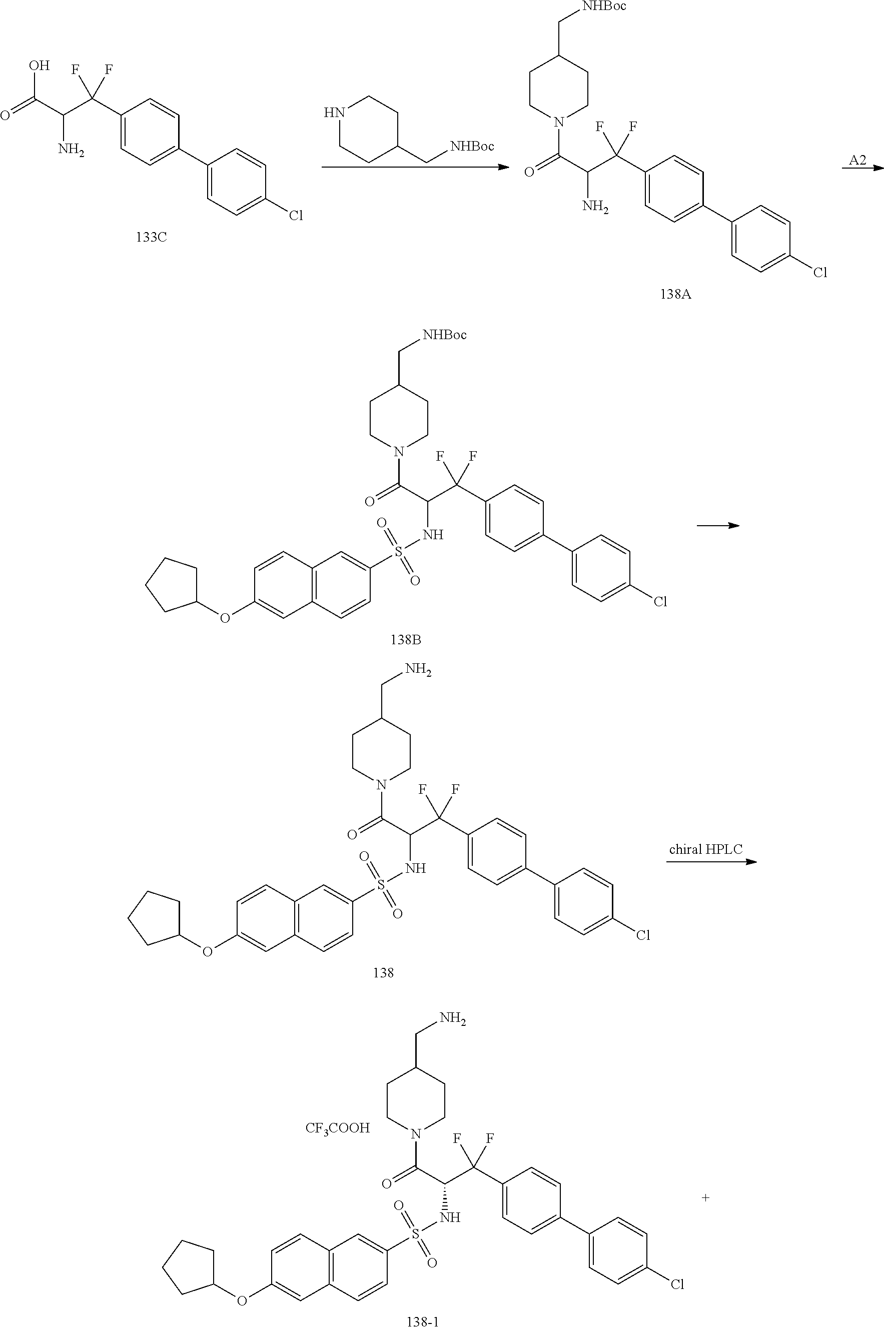

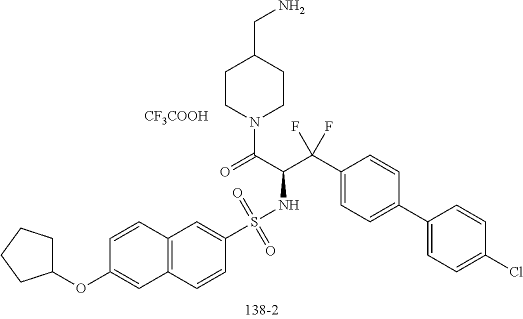 [SMILES: NCC1CCN(C(=O)[C@H](NS(=O)(=O)c2ccc3cc(OC4CCCC4)ccc3c2)C(F)(F)c2ccc(-c3ccc(Cl)cc3)cc2)CC1 ; "CF3COOH"]

Compounds 138A, 138B, and 138 were synthesized by employing the procedures described for Compound B4, Compounds 2D, and 1 using tert-butyl piperidin-4-ylmethylcarbamate, Compounds 133C, 138A, Compound A2, and Compound 138B in lieu of Compounds B4-1, B2-6, B2, Compounds 2C, and 1A.

Compound 138A: LC-MS (ESI) m/z: 508 [M+H]⁺.

Compound 138 was separated with chiral HPLC to give Compound 138-1 and Compound 138-2.

Compound 138-1: LC-MS (ESI) m/z: 682 [M+H]⁺; ¹H-NMR (DMSO-d₆, 400 MHz): δ (ppm) 0.53-0.75 (m, 1H), 1.32-1.88 (m, 9H), 1.90-2.12 (m, 2H), 2.24-2.46 (m, 4H), 2.58-2.82 (m, 1H), 3.82-4.17 (m, 2H), 4.77-5.19 (m, 2H), 7.12-7.33 (m, 2H), 7.46-7.76 (m, 12H), 7.80 (dd, J=8.8, 3.8 Hz, 1H), 7.87-8.00 (m, 1H), 8.16 (d, J=11.3 Hz, 1H), 8.62-8.50 (m, 1H); ¹⁹F-NMR (376 MHz, DMSO-d₆): δ (ppm) −73.52 (s). Chiral separation condition: MeOH contained 0.5% NH₄OH; (R, R)-Whelk-O1 (4.6×250 mm, 5 μm); retention time: 8.04 minute.

Compound 138-2: LC-MS (ESI) m/z: 682 [M+H]⁺; ¹H-NMR (DMSO-d₆, 400 MHz): δ (ppm) 0.59-0.61 (m, 1H), 1.36-1.85 (m, 9H), 1.90-2.16 (m, 2H), 2.27-2.49 (m, 4H), 2.59-2.82 (m, 1H), 3.82-4.15 (m, 2H), 4.80-5.16 (m, 2H), 7.13-7.21 (m, 1H), 7.22-7.29 (m, 1H), 7.77-7.48 (m, 12H), 7.77-7.85 (m, 1H), 7.92 (dd, J=16.7, 9.1 Hz, 1H), 8.16 (d, J=11.7 Hz, 1H), 8.56 (dd, J=10.1, 4.0 Hz, 1H); ¹⁹F-NMR (376 MHz, DMSO-d₆): δ (ppm) −73.51 (s). Chiral separation condition: MeOH contained 0.5% NH₄OH; (R, R)-Whelk-O1 (4.6×250 mm, 5 μm); retention time: 6.5 minute.

### Example 139

### Synthesis of N—((R)-3-((R)-3-aminopyrrolidin-1-yl)-1-(4-bromophenyl)-1,1-difluoro-3-oxopropan-2-yl)-6-(cyclopentyloxy)naphthalene-2-sulfonamide (Compound 139-1), and N—((S)-3-((R)-3-aminopyrrolidin-1l-yl)-1-(4-bromophenyl)-1,1-difluoro-3-oxopropan-2-yl)-6-(cyclopentyloxy)naphthalene-2-sulfonamide (Compound 139-2)

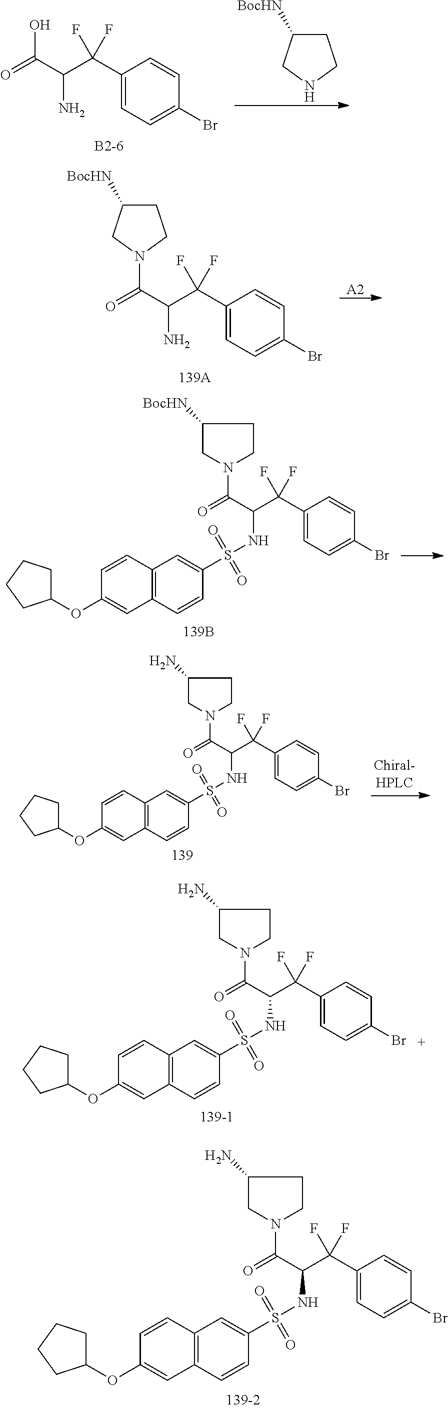

Compounds 139A, 139B, and 139 were synthesized by employing the procedures described for Compound B4, Compounds 2D, and 1 using (R)-tert-butyl pyrrolidin-3-ylcarbamate, Compound 139A, Compound A2, and Compound 139B in lieu of Compounds B4-1, B2, 2C, and 1A.

Compound 139A: LC-MS (ESI) m/z: 448.1 [M+H]⁺.

Compound 139B: LC-MS (ESI) m/z: 666.1 [M−56+H]⁺.

Compound 139 was separated with chiral HPLC to furnish Compound 139-1 and Compound 139-2.

Compound 139-1: LC-MS (ESI) m/z: 622.1 [M+H]⁺; ¹H-NMR (CD₃OD, 400 MHz): δ (ppm) 1.26-2.02 (m, 10H), 2.56-2.66 (m, 1H), 2.70-3.00 (m, 2H), 3.05-3.19 (m, 1H), 3.26-3.45 (m, 1H), 4.39-4.47 (m, 1H), 4.87-4.94 (m, 1H), 7.10-7.16 (m, 1H), 7.18-7.30 (m, 3H), 7.34-7.44 (m, 2H), 7.48-7.53 (m, 1H), 7.68-7.72 (m, 1H), 7.76-7.83 (m, 1H), 8.09-8.13 (m, 1H). Chiral separation condition: MeOH contained 0.2% Methanol Ammonia; OZ-H (4.6×250 mm, 5 μm); retention time: 13.31 minute.

Compound 139-2: LC-MS (ESI) m/z: 622.1 [M+H]⁺; ¹H-NMR (CD₃OD, 400 MHz): δ (ppm) 1.34-2.12 (m, 10H), 2.56-2.88 (m, 1H), 3.08-3.20 (m, 2H), 3.34-3.68 (m, 2H), 4.40-4.64 (m, 1H), 4.87-4.94 (m, 1H), 7.10-7.30 (m, 5H), 7.36-7.52 (m, 2H), 7.63-7.81 (m, 2H), 8.04-8.14 (m, 1H). Chiral separation condition: MeOH contained 0.2% Methanol Ammonia; OZ-H (4.6×250 mm, 5 m); retention time: 9.89 minute.

### Example 140

### Synthesis of N—((R)-3-((S)-3-aminopyrrolidin-1-yl)-1-(4-bromophenyl)-1,1-difluoro-3-oxopropan-2-yl)-6-(cyclopentyloxy)naphthalene-2-sulfonamide (Compound 140-1), and N—((S)-3-((S)-3-aminopyrrolidin-1-yl)-1-(4-bromophenyl)-1,1-difluoro-3-oxopropan-2-yl)-6-(cyclopentyloxy)naphthalene-2-sulfonamide (Compound 140-2)

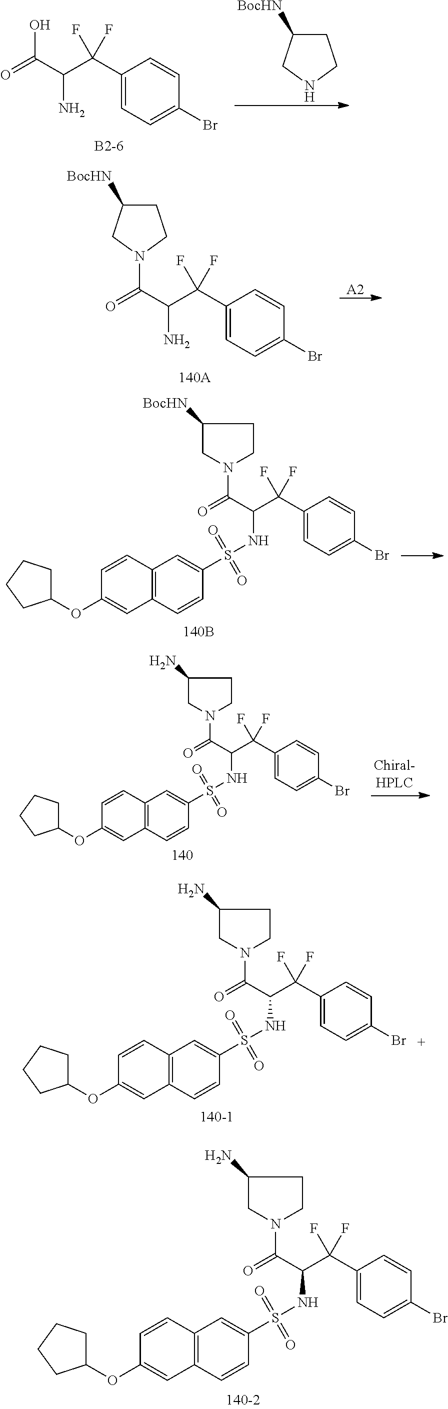

Compounds 140A, 140B, and 140 were synthesized by employing the procedures described for Compound B4, Compounds 2D, and 1 using (S)-tert-butyl pyrrolidin-3-ylcarbamate, Compound 140A, Compound A2, and Compound 140B in lieu of Compounds B4-1, B2, 2C, and 1A.

Compound 140A: LC-MS (ESI) m/z: 448 [M+H]⁺.

Compound 140B: LC-MS (ESI) m/z: 666 [M−56+H]⁺.

Compound 140 was separated by chiral HPLC to afford Compound 140-1 and Compound 140-2.

Compound 140-1: LC-MS (ESI) m/z: 622 [M+H]⁺; ¹H-NMR (CD₃OD, 400 MHz): δ (ppm) 1.69-1.74 (m, 2H), 1.79-1.95 (m, 5H), 2.04-2.35 (m, 3H), 3.10-3.27 (m, 2H), 3.46-3.73 (m, 2H), 3.80-3.99 (m, 1H), 4.60-4.70 (m, 1H), 4.99-5.03 (m, 1H), 7.19-7.56 (m, 7H), 7.72-7.89 (m, 2H), 8.08-8.18 (m, 1H). Chiral separation condition: n-Hexane/EtOH contained 0.1% DEA; OJ-H (4.6×250 mm, 5 μm); retention time 15.94 minute.

Compound 140-2: LC-MS (ESI) m/z: 622 [M+H]⁺; ¹H-NMR (CD₃OD, 400 MHz): δ (ppm) 1.75-2.12 (m, 9H), 2.39-2.92 (m, 1H), 3.32-3.39 (m, 1H), 3.41-3.62 (m, 1H), 3.66-3.75 (m, 1H), 3.79-4.01 (m, 2H), 4.54-4.78 (m, 1H), 5.01-5.03 (m, 1H), 7.17-7.89 (m, 9H), 8.08-8.18 (m, 1H). Chiral separation condition: n-Hexane/EtOH contained 0.1% DEA; OJ-H (4.6×250 mm, 5 μm); retention time: 9.59 minute.

### Example 141

### Synthesis of (R)—N—((R)-3-(4-aminopiperidin-1-yl)-1-(4′-chloro-[1,1′-biphenyl]-4-yl)-1,1-difluoro-3-oxopropan-2-yl)-2-(4-(cyclohexylmethoxy)phenyl)-2-hydroxyacetamide trifluoroacetate (Compound 141-1), (S)—N—((S)-3-(4-aminopiperidin-1-yl)-1-(4′-chloro-[1,1′-biphenyl]-4-yl)-1,1-difluoro-3-oxopropan-2-yl)-2-(4-(cyclohexylmethoxy)phenyl)-2-hydroxyacetamide trifluoroacetate (Compound 141-2), (S)—N—((R)-3-(4-aminopiperidin-1-yl)-1-(4′-chloro-[1,1′-biphenyl]-4-yl)-1,1-difluoro-3-oxopropan-2-yl)-2-(4-(cyclohexylmethoxy)phenyl)-2-hydroxyacetamide trifluoroacetate (Compound 141-3), and (R)—N—((S)-3-(4-aminopiperidin-1-yl)-1-(4′-chloro-[1,1′-biphenyl]-4-yl)-1,1-difluoro-3-oxopropan-2-yl)-2-(4-(cyclohexylmethoxy)phenyl)-2-hydroxyacetamide trifluoroacetate (Compound 141-4)

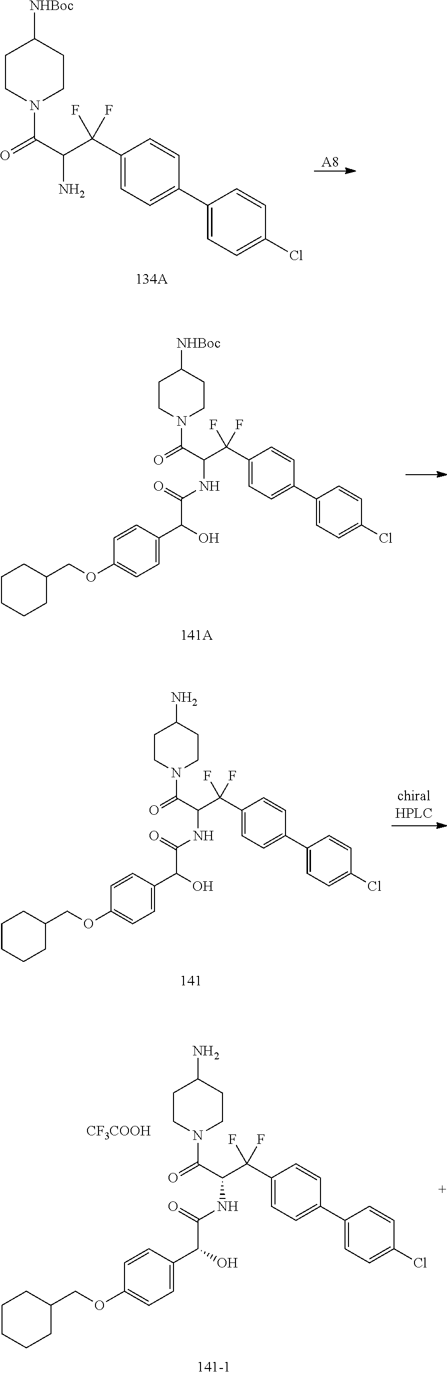 [SMILES: CC(C)(C)OC(=O)NC1CCN(C(=O)C(N)C(F)(F)c2ccc(-c3ccc(Cl)cc3)cc2)CC1 ; "A8" ; CC(C)(C)OC(=O)NC1CCN(C(=O)C(NC(=O)C(O)c2ccc(OCC3CCCCC3)cc2)C(F)(F)c2ccc(-c3ccc(Cl)cc3)cc2)CC1 ; NC1CCN(C(=O)C(NC(=O)C(O)c2ccc(OCC3CCCCC3)cc2)C(F)(F)c2ccc(-c3ccc(Cl)cc3)cc2)CC1 ; NC1CCN(C(=O)[C@@H](NC(=O)[C@H](O)c2ccc(OCC3CCCCC3)cc2)C(F)(F)c2ccc(-c3ccc(Cl)cc3)cc2)CC1 ; "CF3COOH"]

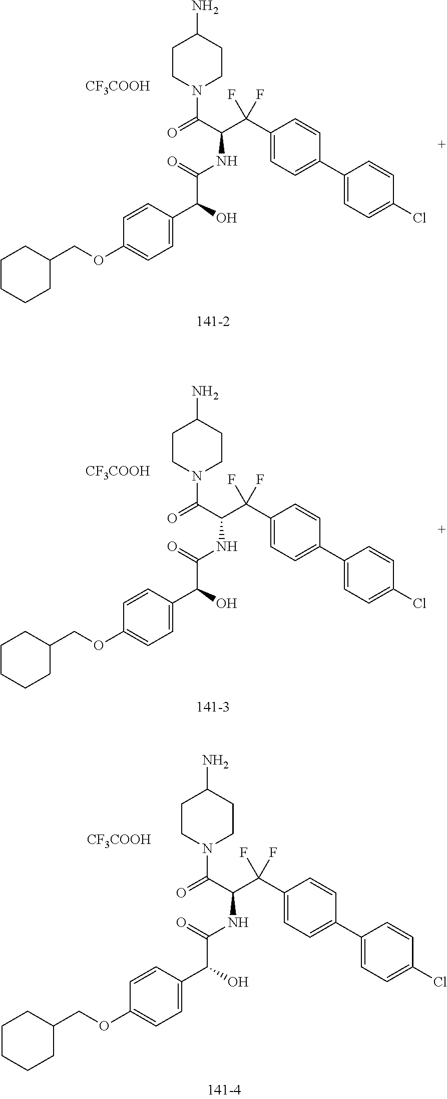 [SMILES: NC1CCN(C(=O)[C@H](NC(=O)[C@@H](O)c2ccc(OCC3CCCCC3)cc2)C(F)(F)c2ccc(-c3ccc(Cl)cc3)cc2)CC1 ; "CF3COOH" ; NC1CCN(C(=O)[C@@H](NC(=O)[C@@H](O)c2ccc(OCC3CCCCC3)cc2)C(F)(F)c2ccc(-c3ccc(Cl)cc3)cc2)CC1 ; "CF3COOH" ; NC1CCN(C(=O)[C@H](NC(=O)[C@H](O)c2ccc(OCC3CCCCC3)cc2)C(F)(F)c2ccc(-c3ccc(Cl)cc3)cc2)CC1 ; "CF3COOH"]

Compounds 141A and 141 were synthesized by employing the procedures described for Compound B4 and Compound 1 using Compound 134A using DMF as solvent and without DIPEA, Compound A8, and Compound 141A in lieu of Compounds B4-1 using dichloromethane as solvent and with DIPEA, B2-6, and 1A.

Compound 141A: LC-MS (ESI) m/z: 684 [M−56+H]⁺.

Compound 141 was separated with chiral HPLC to give Compound 141-1, Compound 141-2, Compound 141-3, and Compound 141-4.

Compound 141-1: LC-MS (ESI) m/z: 640 [M+H]⁺; ¹H-NMR (CD₃OD, 400 MHz): δ (ppm) 0.98-1.05 (m, 2H), 1.29-1.81 (m, 12H), 2.01-2.12 (m, 2H), 2.76-3.40 (m, 2H), 3.60-3.64 (m, 2H), 4.21-4.30 (m, 1H), 4.61-4.68 (m, 1H), 4.94-5.0 (m, 1H), 5.72-5.78 (m, 1H), 6.74-6.79 (m, 2H), 7.16-7.22 (m, 2H), 7.48-7.53 (m, 4H), 7.60-7.64 (m, 4H). Chiral separation condition: MeOH contained 0.2% Methanol Ammonia; R,R-Whelk-O1 (4.6×250 mm, 5 μm); retention time: 10.06 minute.

Compound 141-2: LC-MS (ESI) m/z: 640 [M+H]⁺; ¹H-NMR (CD₃OD, 400 MHz): δ (ppm) 0.97-1.05 (m, 2H), 1.30-1.80 (m, 12H), 2.01-2.12 (m, 2H), 2.76-3.40 (m, 2H), 3.60-3.64 (m, 2H), 4.21-4.30 (m, 1H), 4.61-4.68 (m, 1H), 4.94-5.0 (m, 1H), 5.72-5.78 (m, 1H), 6.74-6.79 (m, 2H), 7.16-7.22 (m, 2H), 7.48-7.53 (m, 4H), 7.60-7.65 (m, 4H). Chiral separation condition: MeOH contained 0.2% Methanol Ammonia; R,R-Whelk-O1 (4.6×250 mm, 5 μm); 3.96 minute.

Compound 141-3: LC-MS (ESI) m/z: 640 [M+H]⁺; ¹H-NMR (CD₃OD, 400 MHz): δ (ppm) 0.97-1.04 (m, 2H), 1.23-1.33 (m, 3H), 1.53-1.78 (m, 8H), 2.06-2.20 (m, 2H), 2.82-3.21 (m, 2H), 3.41-3.51 (m, 3H), 4.33-4.41 (m, 1H), 4.66-4.77 (m, 1H), 4.88-4.95 (m, 1H), 5.79-5.84 (m, 1H), 6.60-6.66 (m, 2H), 6.91-6.95 (m, 2H), 7.47-7.50 (m, 2H), 7.56-7.64 (m, 6H). Chiral separation condition: MeOH contained 0.2% Methanol Ammonia; IC (4.6×150 mm, 5 μm); retention time: 3.4 minute.

Compound 141-4: LC-MS (ESI) m/z: 640 [M+H]⁺; ¹H-NMR (CD₃OD, 400 MHz): δ (ppm) 0.98-1.04 (m, 2H), 1.23-1.33 (m, 3H), 1.53-1.78 (m, 8H), 2.06-2.20 (m, 2H), 2.82-3.21 (m, 2H), 3.41-3.51 (m, 3H), 4.33-4.41 (m, 1H), 4.66-4.77 (m, 1H), 4.88-4.95 (m, 1H), 5.79-5.84 (m, 1H), 6.60-6.66 (m, 2H), 6.90-6.95 (m, 2H), 7.47-7.50 (m, 2H), 7.56-7.65 (m, 6H). Chiral separation condition: MeOH contained 0.2% Methanol Ammonia; IC (4.6×150 mm, 5 μm); retention time: 5.7 minute.

### Example 142

### Synthesis of N-((2R)-3-(3-amino-8-azabicyclo[3.2.1]octan-8-yl)-1-(4′-chloro-2-methoxy-[1,1′-biphenyl]-4-yl)-1,1-difluoro-3-oxopropan-2-yl)-4-(cyclohexylmethoxy) benzenesulfonamide trifluoroacetate (Compound 142-1), and N-((2S)-3-(3-amino-8-azabicyclo[3.2.1]octan-8-yl)-1-(4′-chloro-2-methoxy-[1,1′-biphenyl]-4-yl)-1,1-difluoro-3-oxopropan-2-yl)-4-(cyclohexylmethoxy)benzenesulfonamide trifluoroacetate (Compound 142-2)

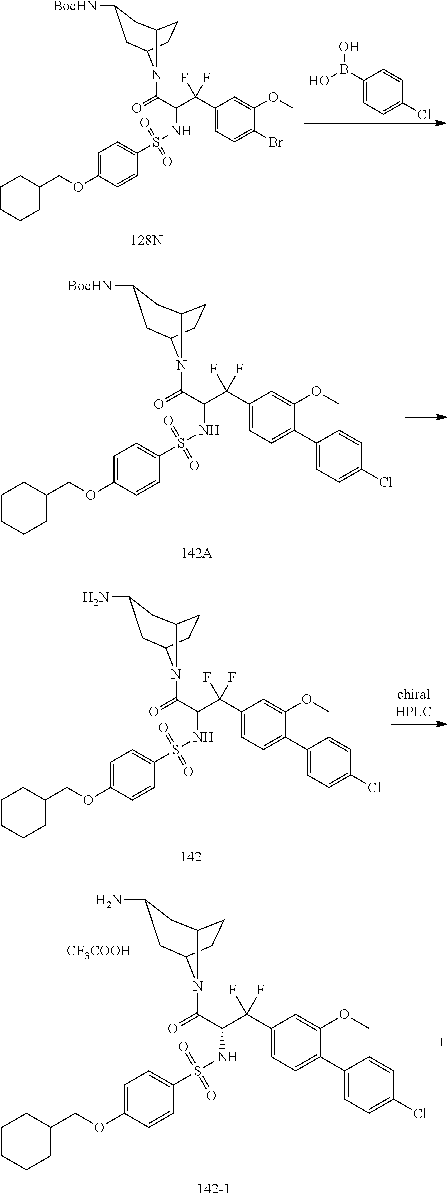 [SMILES: COc1cc(C(F)(F)C(NS(=O)(=O)c2ccc(OCC3CCCCC3)cc2)C(=O)N2C3CCC2CC(NC(=O)OC(C)(C)C)C3)ccc1Br ; OB(O)c1ccc(Cl)cc1 ; COc1cc(C(F)(F)C(NS(=O)(=O)c2ccc(OCC3CCCCC3)cc2)C(=O)N2C3CCC2CC(NC(=O)OC(C)(C)C)C3)ccc1-c1ccc(Cl)cc1 ; COc1cc(C(F)(F)C(NS(=O)(=O)c2ccc(OCC3CCCCC3)cc2)C(=O)N2C3CCC2CC(N)C3)ccc1-c1ccc(Cl)cc1 ; COc1cc(C(F)(F)[C@H](NS(=O)(=O)c2ccc(OCC3CCCCC3)cc2)C(=O)N2C3CCC2CC(N)C3)ccc1-c1ccc(Cl)cc1 ; "CF3COOH"]

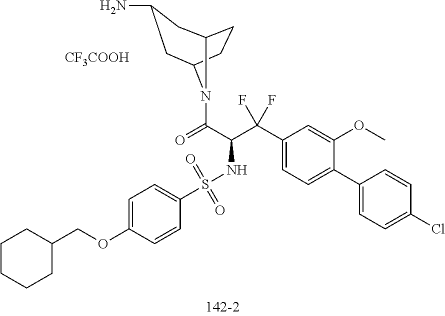 [SMILES: COc1cc(C(F)(F)[C@@H](NS(=O)(=O)c2ccc(OCC3CCCCC3)cc2)C(=O)N2C3CCC2CC(N)C3)ccc1-c1ccc(Cl)cc1 ; "CF3COOH"]

Compounds 142A and 142 were synthesized by employing the procedures described for Compounds 14C and 1 using 4-chlorophenylboronic acid, Compounds 128N, and 142A in lieu of Compounds 14B, 14A, and 1A.

Compound 142A: LC-MS (ESI) m/z: non-ionizable compound under routine conditions used.

Compound 142 was separated with chiral HPLC to give Compound 142-1 and Compound 142-2.

Compound 142-1: LC-MS (ESI) m/z: 702 [M+H]⁺; ¹H-NMR (CD₃OD, 400 MHz): δ (ppm) 0.88-1.36 (m, 6H), 1.60-2.20 (m, 13H), 3.45-3.77 (m, 6H), 4.67-4.85 (m, 3H), 6.76-7.14 (m, 4H), 7.27-7.33 (m, 1H), 7.42-7.57 (m, 6H). Chiral separation condition: n-Hexane/EtOH contained 0.1% DEA, 50/50; AD-H (250×4.6 mm, 5 μm); retention time: 14.03 minute.

Compound 142-2: LC-MS (ESI) m/z: 702 [M+H]⁺; ¹H-NMR (CD₃OD, 400 MHz): δ (ppm) 0.88-1.36 (m, 6H), 1.60-2.20 (m, 13H), 3.45-3.77 (m, 6H), 4.67-4.85 (m, 3H), 6.76-7.14 (m, 4H), 7.27-7.33 (m, 1H), 7.42-7.57 (m, 6H). Chiral separation condition: n-Hexane/EtOH contained 0.1% DEA, 50/50; AD-H (250×4.6 mm, 5 μm); retention time: 9.56 minute.

### Example 143

### Synthesis of (R)—N-(3-(4-aminopiperidin-1-yl)-1-(4-bromo-3-methoxyphenyl)-1,1-difluoro-3-oxopropan-2-yl)-6-(cyclopentyloxy)naphthalene-2-sulfonamide trifluoroacetate (Compound 143-1), and (S)—N-(3-(4-aminopiperidin-1-yl)-1-(4-bromo-3-methoxyphenyl)-1,1-difluoro-3-oxopropan-2-yl)-6-(cyclopentyloxy) naphthalene-2-sulfonamide trifluoroacetate (Compound 143-2)

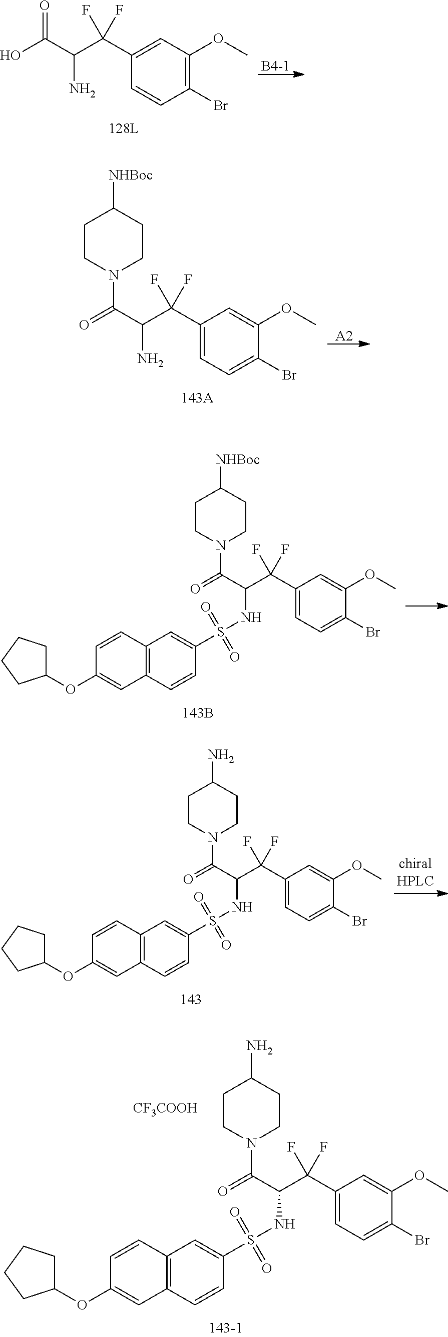

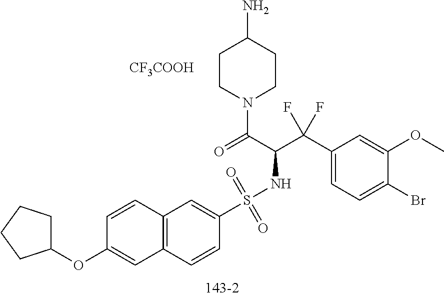 [SMILES: COc1cc(C(F)(F)[C@@H](NS(=O)(=O)c2ccc3cc(OC4CCCC4)ccc3c2)C(=O)N2CCC(N)CC2)ccc1Br ; "CF3COOH"]

Compounds 143A, 143B, and 143 were synthesized by employing the procedures described for Compound B4, Compounds 2D, and 1 using Compounds 128L, 143A, Compound A2, and Compound 143B in lieu of Compounds B2-6, B2, Compounds 2C, and 1A.

Compound 143A: LC-MS (ESI) m/z: 492 [M+H]⁺.

Compound 143B: LC-MS (ESI) m/z: 710 [M−56+H]⁺.

Compound 143 was separated with chiral HPLC to give Compound 143-1 and Compound 143-2.

Compound 143-1: LC-MS (ESI) m/z: 666 [M+H]⁺; ¹H-NMR (CD₃OD, 500 MHz): δ (ppm) 1.35-1.49 (m, 2H), 1.73-2.18 (m, 10H), 2.80-3.04 (m, 1H), 3.23-3.24 (m, 1H), 3.45-3.46 (m, 1H), 3.59-3.77 (m, 3H), 4.23-4.25 (m, 1H), 4.38-4.63 (m, 1H), 5.02-5.04 (m, 2H), 6.82-6.86 (m, 1H), 6.95-7.01 (m, 1H), 7.25-7.38 (m, 3H), 7.46-7.50 (m, 1H), 7.66-7.74 (m, 1H), 7.81-7.86 (m, 1H), 8.08-8.16 (m, 1H). Chiral separation condition: MeOH contained 1% Methanol Ammonia; R, R-Whelk-O1 (4.6×250 mm, 5 μm); retention time: 4.85 minute.

Compound 143-2: LC-MS (ESI) m/z: 666 [M+H]⁺; ¹H-NMR (CD₃OD, 500 MHz): δ (ppm) 0.98-1.05 (m, 2H), 1.29-1.81 (m, 10H), 2.76-3.45 (m, 3H), 3.60-3.77 (m, 3H), 4.24-4.30 (m, 1H), 4.61-4.68 (m, 1H), 4.94-5.0 (m, 2H), 6.82-7.02 (m, 2H), 7.29-7.45 (m, 4H), 7.71-7.88 (m, 2H), 8.08-8.17 (m, 1H). Chiral separation condition: MeOH contained 1% Methanol Ammonia; R, R-Whelk-O1 (4.6×250 mm, 5 μm); retention time: 5.58 minute.

### Example 144

### Synthesis of (R)—N-(3-(4-aminopiperidin-1-yl)-1-(4′-chloro-2-methoxy-[1,1′-biphenyl]-4-yl)-1,1-difluoro-3-oxopropan-2-yl)-6-(cyclopentyloxy)naphthalene-2-sulfonamide trifluoroacetate (Compound 144-1), and (S)—N-(3-(4-aminopiperidin-1-yl)-1-(4′-chloro-2-methoxy-[1,1′-biphenyl]-4-yl)-1,1-difluoro-3-oxopropan-2-yl)-6-(cyclopentyloxy) naphthalene-2-sulfonamide trifluoroacetate (Compound 144-2)

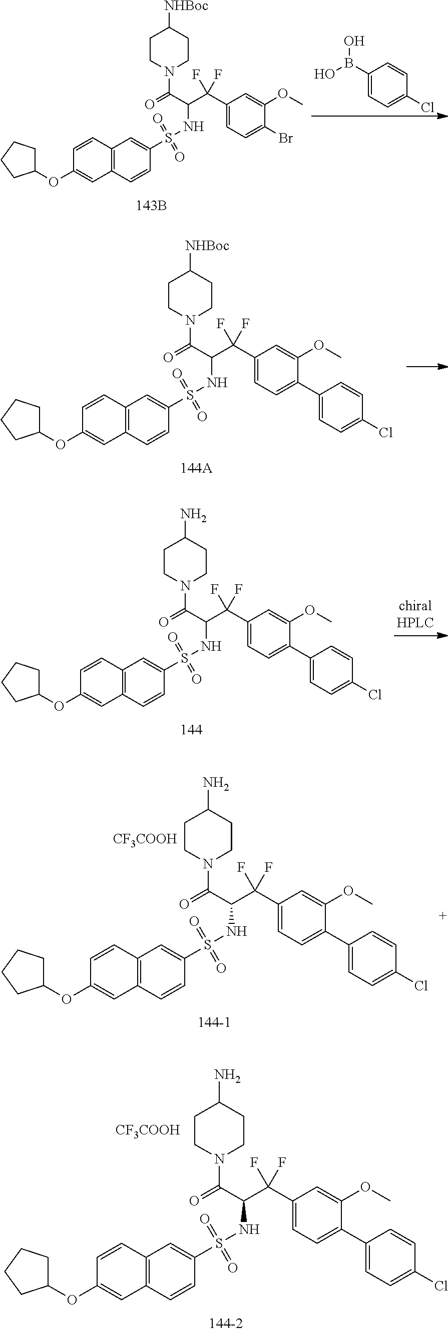

Compounds 144A and 144 were synthesized by employing the procedures described for Compounds 14C and 1 using 4-chlorophenylboronic acid, Compounds 143B, and 144A in lieu of Compounds 14B, 14A, and 1A.

Compound 144A: LC-MS (ESI) m/z: 742 [M−56+H]⁺.

Compound 144 was separated with chiral HPLC to give Compound 144-1 and Compound 144-2.

Compound 144-1: LC-MS (ESI) m/z: 698 [M+H]⁺; ¹H-NMR (CD₃OD, 400 MHz): δ (ppm) 0.76-1.19 (m, 2H), 1.59-1.91 (m, 10H), 2.09-2.54 (m, 1H), 2.82-2.92 (m, 2H), 3.56-3.61 (m, 3H), 3.99-4.28 (m, 2H), 4.70-4.76 (m, 1H), 4.85-4.94 (m, 1H), 6.90-7.04 (m, 5H), 7.21-7.27 (m, 4H), 7.41-7.44 (m, 1H), 7.57-7.62 (m, 1H), 7.72-7.75 (m, 1H), 8.04-8.07 (m, 1H). Chiral separation condition: MeOH contained 0.5% NH₄OH; R,R-Whelk-O1 (4.6×250 mm, 5 μm); retention time: 4.55 minute.

Compound 144-2: LC-MS (ESI) m/z: 698 [M+H]⁺; ¹H-NMR (CD₃OD, 400 MHz): δ (ppm) 0.76-1.19 (m, 2H), 1.59-1.91 (m, 10H), 2.09-2.54 (m, 1H), 2.82-2.92 (m, 2H), 3.56-3.61 (m, 3H), 3.99-4.28 (m, 2H), 4.70-4.76 (m, 1H), 4.85-4.94 (m, 1H), 6.90-7.04 (m, 5H), 7.21-7.27 (m, 4H), 7.41-7.44 (m, 1H), 7.57-7.62 (m, 1H), 7.72-7.75 (m, 1H), 8.04-8.07 (m, 1H). Chiral separation condition: MeOH contained 0.5% NH₄OH; R,R-Whelk-O1 (4.6×250 mm, 5 μm); retention time: 5.47 minute.

### Example 145

### Synthesis of (R)—N-(3-(4-aminopiperidin-1-yl)-1-(4-bromo-3-methoxyphenyl)-1,1-difluoro-3-oxopropan-2-yl)-4-(cyclohexylmethoxy)benzenesulfonamide trifluoroacetate (Compound 145-1), and (S)—N-(3-(4-aminopiperidin-1-yl)-1-(4-bromo-3-methoxyphenyl)-1,1-difluoro-3-oxopropan-2-yl)-4-(cyclohexylmethoxy) benzenesulfonamide trifluoroacetate (Compound 145-2)

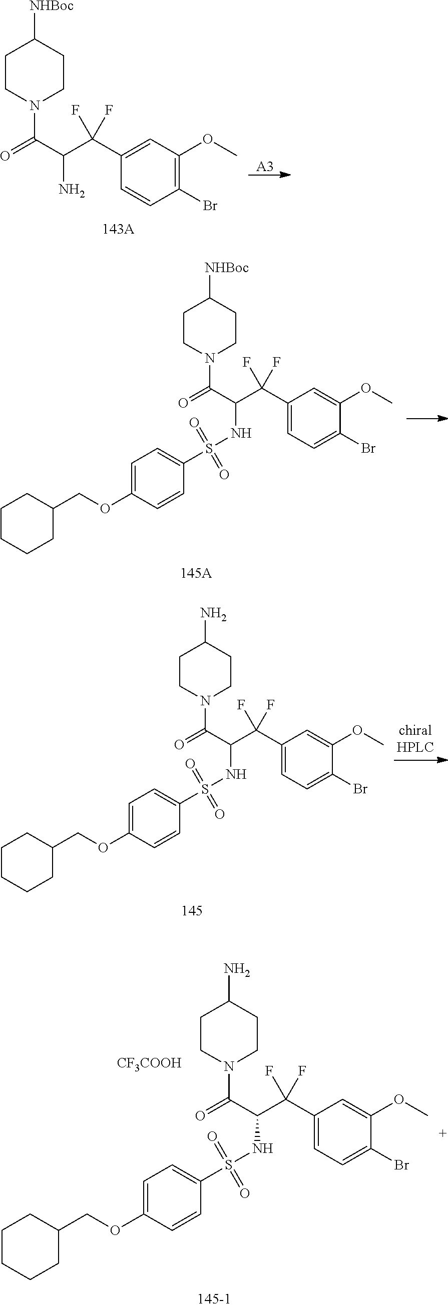

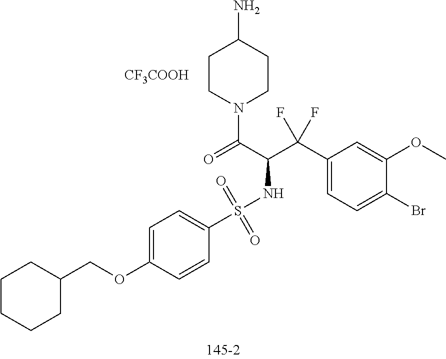 [SMILES: COc1cc(C(F)(F)[C@@H](NS(=O)(=O)c2ccc(OCC3CCCCC3)cc2)C(=O)N2CCC(N)CC2)ccc1Br ; "CF3COOH"]

Compounds 145A and 145 were synthesized by employing the procedures described for Compounds 2D and 1 using Compound 143A, Compound A3, and Compound 145A in lieu of Compound B2, Compounds 2C, and 1A.

Compound 145A: LC-MS (ESI) m/z: 688 [M−56+H]⁺.

Compound 145 was separated with chiral HPLC to give Compound 145-1 and Compound 145-2.

Compound 145-1: LC-MS (ESI) m/z: 644 [M+H]⁺; ¹H-NMR (CD₃OD, 500 MHz): δ (ppm) 1.16-1.27 (m, 3H), 1.36-1.59 (m, 3H), 1.74-2.15 (m, 8H), 2.57-2.81 (m, 1H), 3.15-3.24 (m, 1H), 3.39-3.45 (m, 1H), 3.79-3.88 (m, 5H), 4.26-4.63 (m, 2H), 4.94-4.99 (m, 2H), 6.86-6.90 (m, 3H), 6.99-7.03 (m, 1H), 7.43-7.45 (m, 1H), 7.50-7.57 (m, 2H). Chiral separation condition: MeOH contained 0.2% Methanol Ammonia; OJ-H (4.6×250 mm, 5 μm); retention time: 5.2 minute.

Compound 145-2: LC-MS (ESI) m/z: 644 [M+H]⁺; ¹H-NMR (CD₃OD, 500 MHz): δ (ppm) 1.16-1.27 (m, 3H), 1.36-1.59 (m, 3H), 1.74-2.15 (m, 8H), 2.57-2.81 (m, 1H), 3.15-3.24 (m, 1H), 3.39-3.45 (m, 1H), 3.79-3.88 (m, 5H), 4.26-4.63 (m, 2H), 4.94-4.99 (m, 2H), 6.86-6.90 (m, 3H), 6.99-7.03 (m, 1H), 7.43-7.45 (m, 1H), 7.50-7.57 (m, 2H). Chiral separation condition: MeOH contained 0.2% Methanol Ammonia; OJ-H (4.6×250 mm, 5 μm); retention time: 3.52 minute.

### Example 146

### Synthesis of (R)—N-(3-(4-aminopiperidin-1-yl)-1-(4′-chloro-2-methoxy-[1,1′-biphenyl]-4-yl)-1,1-difluoro-3-oxopropan-2-yl)-4-(cyclohexylmethoxy) benzenesulfonamide trifluoroacetate (Compound 146-1), and (S)—N-(3-(4-aminopiperidin-1-yl)-1-(4′-chloro-2-methoxy-[1,1′-biphenyl]-4-yl)-1,1-difluoro-3-oxopropan-2-yl)-4-(cyclohexylmethoxy)benzenesulfonamide trifluoroacetate (Compound 146-2)

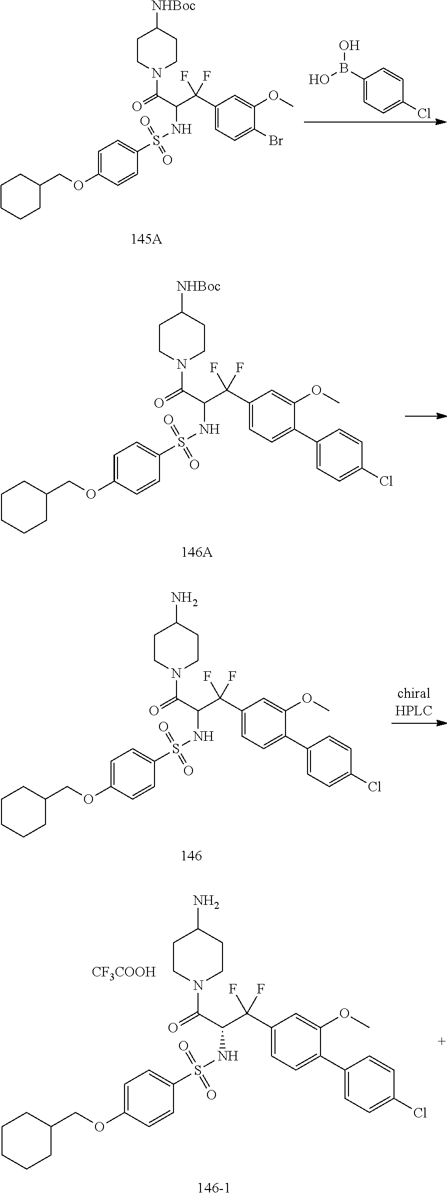 [SMILES: COc1cc(C(F)(F)C(NS(=O)(=O)c2ccc(OCC3CCCCC3)cc2)C(=O)N2CCC(NC(=O)OC(C)(C)C)CC2)ccc1Br ; OB(O)c1ccc(Cl)cc1 ; COc1cc(C(F)(F)C(NS(=O)(=O)c2ccc(OCC3CCCCC3)cc2)C(=O)N2CCC(NC(=O)OC(C)(C)C)CC2)ccc1-c1ccc(Cl)cc1 ; COc1cc(C(F)(F)C(NS(=O)(=O)c2ccc(OCC3CCCCC3)cc2)C(=O)N2CCC(N)CC2)ccc1-c1ccc(Cl)cc1 ; COc1cc(C(F)(F)[C@H](NS(=O)(=O)c2ccc(OCC3CCCCC3)cc2)C(=O)N2CCC(N)CC2)ccc1-c1ccc(Cl)cc1 ; "CF3COOH"]

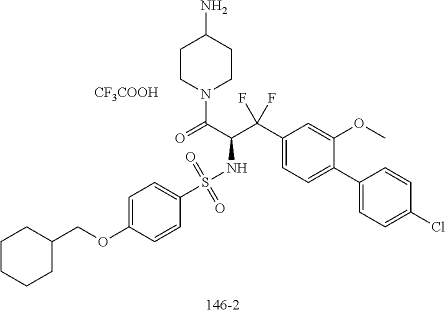 [SMILES: COc1cc(C(F)(F)[C@@H](NS(=O)(=O)c2ccc(OCC3CCCCC3)cc2)C(=O)N2CCC(N)CC2)ccc1-c1ccc(Cl)cc1 ; "CF3COOH"]

Compounds 146A and 146 were synthesized by employing the procedures described for Compounds 14C and 1 using 4-chlorophenylboronic acid, Compounds 145A, and 146A in lieu of Compounds 14B, 14A, and 1A.

Compound 146A: LC-MS (ESI) m/z: 720 [M−56+H]⁺.

Compound 146 was separated with chiral HPLC to give Compound 146-1 and Compound 146-2.

Compound 146-1: LC-MS (ESI) m/z: 676[M+H]⁺; ¹H-NMR (CD₃OD, 500 MHz): δ (ppm) 0.98-1.26 (m, 4H), 1.48-1.77 (m, 8H), 2.05-2.11 (m, 1H), 2.18-2.20 (m, 1H), 2.65-2.85 (m, 1H), 3.22-3.30 (m, 1H), 3.32-3.42 (m, 2H), 3.50-3.57 (m, 1H), 3.70-3.76 (m, 3H), 4.30-4.66 (m, 2H), 4.90-4.98 (m, 2H), 6.78-6.83 (m, 2H), 6.91-7.03 (m, 1H), 7.09-7.14 (m, 1H), 7.28-7.34 (m, 1H), 7.44-7.57 (m, 6H). Chiral separation condition: n-Hexane/EtOH contained 0.1% DEA, 60/40; (S,S)-Whelk-O1 (4.6×250 mm, 5 μm); retention time: 14.8 min.

Compound 146-2: LC-MS (ESI) m/z: 676 [M+H]⁺; ¹H-NMR (CD₃OD, 500 MHz): δ (ppm) 0.98-1.26 (m, 4H), 1.48-1.77 (m, 8H), 2.05-2.11 (m, 1H), 2.18-2.20 (m, 1H), 2.65-2.85 (m, 1H), 3.22-3.30 (m, 1H), 3.32-3.42 (m, 2H), 3.50-3.57 (m, 1H), 3.70-3.76 (m, 3H), 4.30-4.66 (m, 2H), 4.90-4.98 (m, 2H), 6.78-6.83 (m, 2H), 6.91-7.03 (m, 1H), 7.09-7.14 (m, 1H), 7.28-7.34 (m, 1H), 7.44-7.57 (m, 6H). Chiral separation condition: n-Hexane/EtOH contained 0.1% DEA, 60/40; (S,S)-Whelk-O1 (4.6×250 mm, 5 μm); retention time: 15.9 minute.

### Example 147

### Synthesis of N—((R)-1-(4′-chloro-[1,1′-biphenyl]-4-yl)-1,1-difluoro-3-((3aR,6aS)-hexahydropyrrolo[3,4-c]pyrrol-2(1H)-yl)-3-oxopropan-2-yl)-6-(cyclopentyloxy) naphthalene-2-sulfonamide trifluoroacetate (Compound 147-1), and N—((S)-1-(4′-chloro-[1,1′-biphenyl]-4-yl)-1,1-difluoro-3-((3aR,6aS)-hexahydropyrrolo[3,4-c]pyrrol-2(1H)-yl)-3-oxopropan-2-yl)-6-(cyclopentyloxy)naphthalene-2-sulfonamide trifluoroacetate (Compound 147-2)

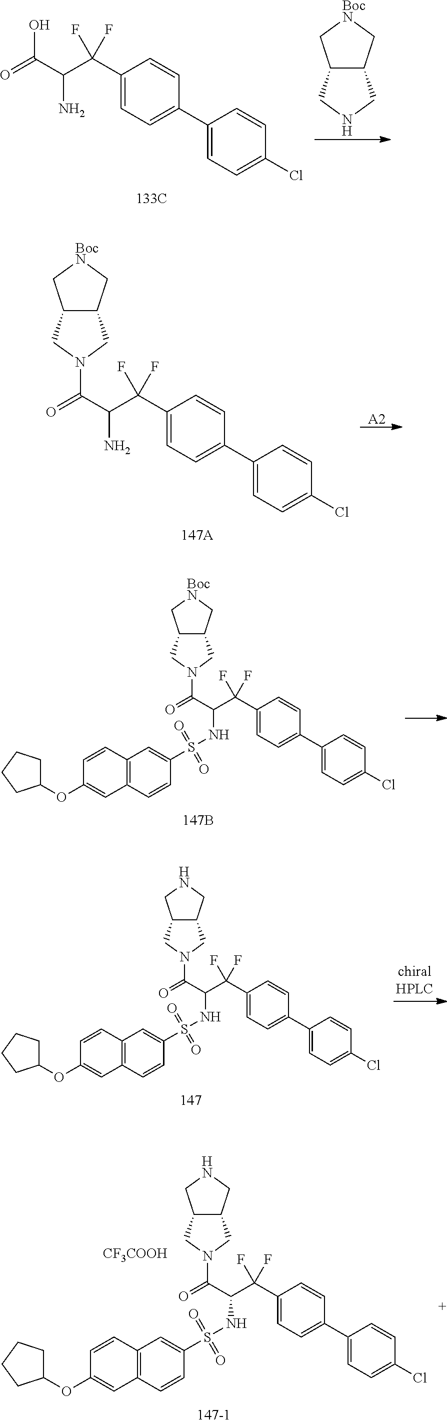

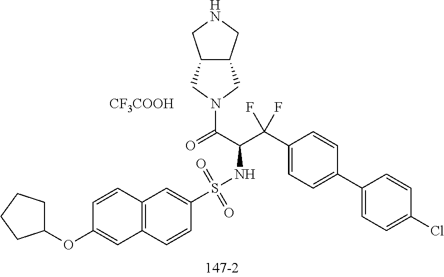 [SMILES: O=C([C@H](NS(=O)(=O)c1ccc2cc(OC3CCCC3)ccc2c1)C(F)(F)c1ccc(-c2ccc(Cl)cc2)cc1)N1C[C@H]2CNC[C@H]2C1 ; "CF3COOH"]

Compounds 147A, 147B, and 147 were synthesized by employing the procedures described for Compound B4, Compounds 2D, and 6 using (3aR, 6aS)-tert-butyl hexahydropyrrolo[3,4-c]pyrrole-2(1H)-carboxylate, Compounds 133C, 147A, Compound A2, and Compound 147B in lieu of Compounds B4-1, B2-6, B2, Compounds 2C, and 6C.

Compound 147A: LC-MS (ESI) m/z: 506 [M+H]⁺.

Compound 147B: LC-MS (ESI) m/z: 780 [M+H]⁺.

Compound 147 was separated with chiral HPLC to furnish Compound 147-1 and Compound 147-2.

Compound 147-1: LC-MS (ESI) m/z: 680 [M+H]⁺; ¹H-NMR (CD₃OD, 400 MHz): δ (ppm) 1.71-1.83 (m, 6H), 2.00-2.03 (m, 2H), 2.66-3.25 (m, 5H), 3.44-3.99 (m, 5H), 4.66-4.83 (m, 2H), 6.89-7.83 (m, 13H), 8.02-8.14 (m, 1H). Chiral separation condition: n-Hexane/EtOH contained 0.1% DEA, 50/50; S,S-Whelk-O1 (4.6×250 mm, 5 μm); retention time: 17.97 minute.

Compound 147-2: LC-MS (ESI) m/z: 680 [M+H]⁺; ¹H-NMR (CD₃OD, 400 MHz): δ (ppm) 1.70-1.83 (m, 6H), 2.00-2.03 (m, 2H), 2.66-3.25 (m, 5H), 3.44-3.99 (m, 5H), 4.68-4.82 (m, 2H), 6.89-7.83 (m, 13H), 8.02-8.14 (m, 1H). Chiral separation condition: n-Hexane/EtOH contained 0.1% DEA, 50/50; S,S-Whelk-O1 (4.6×250 mm, 5 μm); retention time: 12.20 minute.

### Example 148

### Synthesis of N—((R)-3-((S)-3-aminopyrrolidin-1-yl)-1-(4′-chloro-[1,1′-biphenyl]-4-yl)-1,1-difluoro-3-oxopropan-2-yl)-6-(cyclopentyloxy)naphthalene-2-sulfonamide trifluoroacetate (Compound 148-1), and N—((S)-3-((S)-3-aminopyrrolidin-1-yl)-1-(4′-chloro-[1,1′-biphenyl]-4-yl)-1,1-difluoro-3-oxopropan-2-yl)-6-(cyclopentyloxy)naphthalene-2-sulfonamide trifluoroacetate (Compound 148-2)

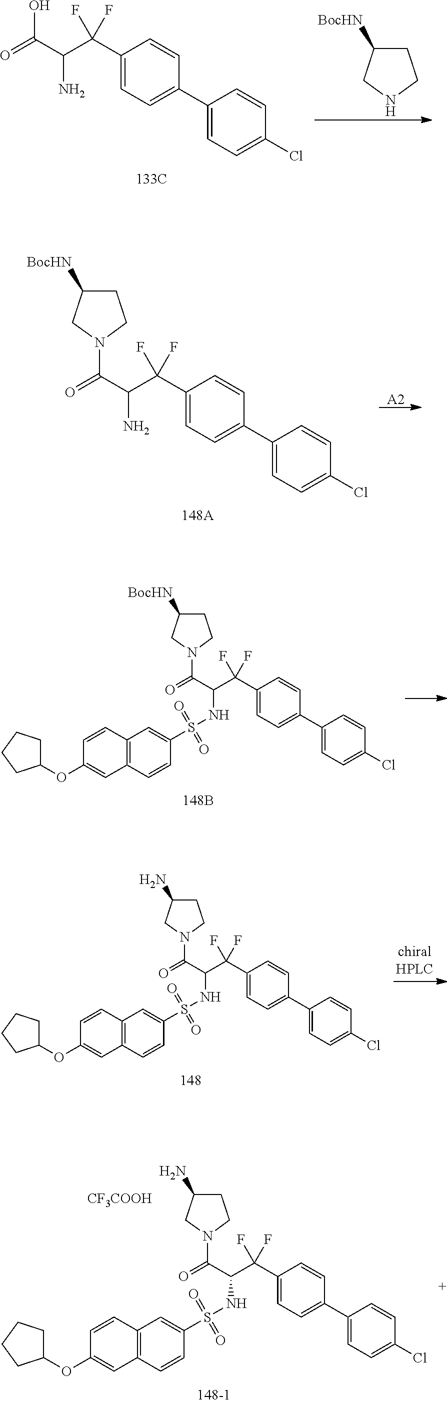

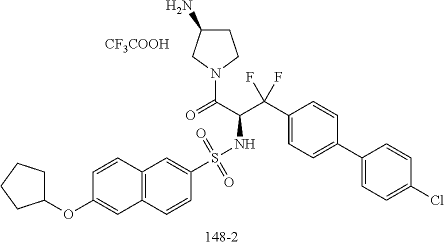 [SMILES: N[C@H]1CCN(C(=O)[C@H](NS(=O)(=O)c2ccc3cc(OC4CCCC4)ccc3c2)C(F)(F)c2ccc(-c3ccc(Cl)cc3)cc2)C1 ; "CF3COOH"]

Compounds 148A, 148B, and 148 were synthesized by employing the procedures described for Compound B4, Compounds 2D, and 1 using (S)-tert-butyl pyrrolidin-3-ylcarbamate, Compounds 133C, 148A, Compound A2, and Compound 148B in lieu of Compounds B4-1, B2-6, B2, Compounds 2C, and 1A.

Compound 148A: LC-MS (ESI) m/z: 480 [M+H]⁺.

Compound 148B: LC-MS (ESI) m/z: 698 [M−55]⁺.

Compound 148 was separated with chiral HPLC to give Compound 148-1 and Compound 148-2.

Compound 148-1: LC-MS (ESI) m/z: 654 [M+H]⁺; ¹H-NMR (CD₃OD, 500 MHz): δ (ppm) 1.71-2.03 (m, 9H), 2.19-2.50 (m, 1H), 3.15-3.57 (m, 2H), 3.73-4.08 (m, 3H), 4.65-4.81 (m, 2H), 6.91-7.10 (m, 2H), 7.16-7.24 (m, 1H), 7.44-7.71 (m, 9H), 7.83-7.95 (m, 1H), 8.02-8.12 (m, 1H). Chiral separation condition: MeOH contained 0.2% Methanol Ammonia; (R,R)-Whelk-O1 (4.6×250 mm, 5 μm); retention time: 5.03 minute.

Compound 148-2: LC-MS (ESI) m/z: 654 [M+H]⁺; ¹H-NMR (CD₃OD, 500 MHz): δ (ppm) 1.71-2.02 (m, 9H), 2.19-2.48 (m, 1H), 3.23-3.55 (m, 2H), 3.73-4.08 (m, 3H), 4.65-4.81 (m, 2H), 6.91-7.10 (m, 2H), 7.16-7.24 (m, 1H), 7.44-7.71 (m, 9H), 7.83-7.95 (m, 1H), 8.02-8.12 (m, 1H). Chiral separation condition: MeOH contained 0.2% Methanol Ammonia; (R,R)-Whelk-O1 (4.6×250 mm, 5 μm); retention time: 6.55 minute.

### Example 149

### Synthesis of N—((R)-3-((R)-3-aminopyrrolidin-1-yl)-1-(4′-chloro-[1,1′-biphenyl]-4-yl)-1,1-difluoro-3-oxopropan-2-yl)-6-(cyclopentyloxy)naphthalene-2-sulfonamide trifluoroacetate (Compound 149-1), and N—((S)-3-((R)-3-aminopyrrolidin-1-yl)-1-(4′-chloro-[1,1′-biphenyl]-4-yl)-1,1-difluoro-3-oxopropan-2-yl)-6-(cyclopentyloxy) naphthalene-2-sulfonamide trifluoroacetate (Compound 149-2)

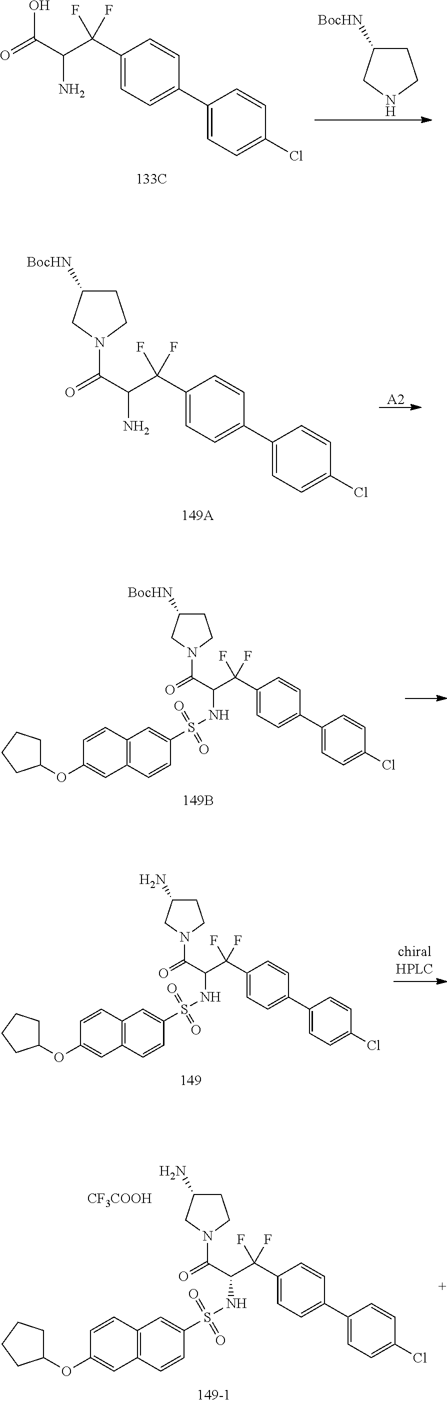

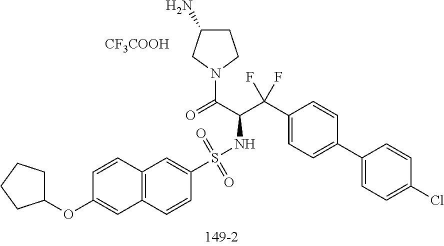 [SMILES: N[C@@H]1CCN(C(=O)[C@H](NS(=O)(=O)c2ccc3cc(OC4CCCC4)ccc3c2)C(F)(F)c2ccc(-c3ccc(Cl)cc3)cc2)C1 ; "CF3COOH"]

Compounds 149A, 149B, and 149 were synthesized by employing the procedures described for Compound B4, Compounds 2D, and 1 using (R)-tert-butyl pyrrolidin-3-ylcarbamate, Compounds 133C, 149A, Compound A2, and Compound 149B in lieu of Compounds B4-1, B2-6, B2, Compounds 2C, and 1A.

Compound 149A: LC-MS (ESI) m/z: 480 [M+H]⁺; ¹H-NMR (CDCl₃, 400 MHz): δ (ppm) 1.45 (s, 9H), 1.75-1.89 (m, 1H), 2.01-2.14 (m, 1H), 3.15-3.81 (m, 4H), 4.04-4.18 (m, 1H), 4.46-4.61 (m, 1H), 7.44 (d, J=8.4 Hz, 2H), 7.53-7.57 (m, 4H), 7.62 (d, J=8.0 Hz, 2H).

Compound 149B: LC-MS (ESI) m/z: 698 [M−55]⁺.

Compound 149 was separated with chiral HPLC to give Compound 149- and Compound 149-2.

Compound 149-1: LC-MS (ESI) m/z: 654 [M+H]⁺; ¹H-NMR (CD₃OD, 400 MHz): δ (ppm) 1.71-2.03 (m, 9H), 2.29-2.39 (m, 1H), 3.21-3.27 (m, 1H), 3.50-3.82 (m, 3H), 3.92-4.04 (m, 1H), 4.70-4.80 (m, 2H), 6.93-7.15 (m, 2H), 7.24-7.53 (m, 9H), 7.59-7.68 (m, 1H), 7.72-7.82 (m, 1H), 8.01-8.12 (m, 1H). Chiral separation condition: MeOH contained 0.2% Methanol Ammonia; (R, R)-Whelk-O1 (4.6×250 mm, 5 μm); retention time: 5.81 minute.

Compound 149-2: LC-MS (ESI) m/z: 654 [M+H]⁺; ¹H-NMR (CD₃OD, 400 MHz): δ (ppm) 1.70-2.04 (m, 9H), 2.14-2.51 (m, 1H), 3.10-3.50 (m, 1H), 3.54-3.73 (m, 1H), 3.81-4.11 (m, 3H), 4.63-4.85 (m, 2H), 6.90-7.15 (m, 2H), 7.21-7.54 (m, 9H), 7.55-7.70 (m, 1H), 7.72-7.82 (m, 1H), 8.01-8.12 (m, 1H). Chiral separation condition: MeOH contained 0.2% Methanol Ammonia; (R, R)-Whelk-O1 (4.6×250 mm, 5 μm); retention time: 8.39 minute.

### Example 150

### Synthesis of N—((R)-3-((1R,5S,8R)-8-amino-3-azabicyclo[3.2.1]octan-3-yl)-1-(4-chlorophenyl)-1,1-difluoro-3-oxopropan-2-yl)-6-(cyclopentyloxy)naphthalene-2-sulfonamide trifluoroacetate (Compound 150-1), and N—((S)-3-((1R,5S,8S)-8-amino-3-azabicyclo[3.2.1]octan-3-yl)-1-(4-chlorophenyl)-1,1-difluoro-3-oxopropan-2-yl)-6-(cyclopentyloxy)naphthalene-2-sulfonamide trifluoroacetate (Compound 150-2)

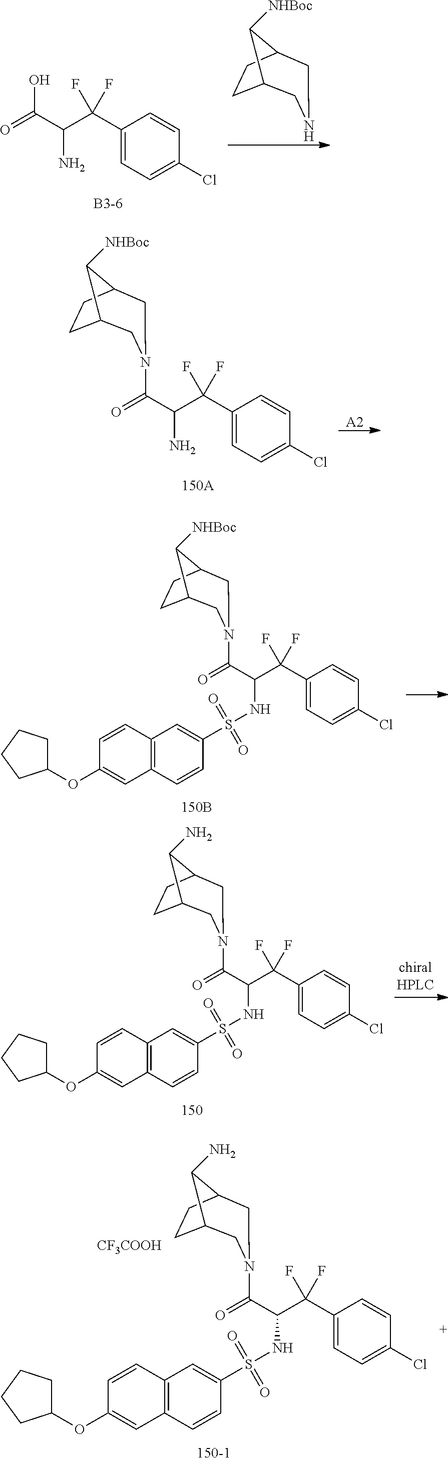

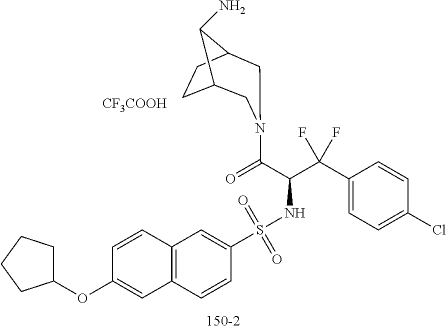 [SMILES: NC1C2CCC1CN(C(=O)[C@H](NS(=O)(=O)c1ccc3cc(OC4CCCC4)ccc3c1)C(F)(F)c1ccc(Cl)cc1)C2 ; "CF3COOH"]

Compounds 150A, 150B, and 150 were synthesized by employing the procedures described for Compound B4, Compounds 2D, and 1 using tert-butyl (1R,5S,8r)-3-azabicyclo[3.2.1]octan-8-ylcarbamate, Compound B3-6, Compound 150A, Compound A2, and Compound 150B in lieu of Compounds B4-1, B2-6, B2, Compounds 2C, and 1A.

Compound 150A: LC-MS (ESI) m/z: 444 [M+H]⁺.

Compound 150B: LC-MS (ESI) m/z: 740 [M+Na]⁺.

Compound 150 was separated with chiral HPLC to afford Compound 150-1 and Compound 150-2.

Compound 150-1: LC-MS (ESI) m/z: 618 [M+H]⁺. ¹H NMR (CD₃OD, 400 MHz): δ (ppm) 0.67-0.74 (m, 1H), 1.14-1.20 (m, 1H), 1.34-1.41 (m, 1H), 1.50-1.60 (m, 1H), 1.67-1.93 (m, 7H), 2.03-2.08 (m, 2H), 2.19-2.47 (m, 2H), 2.91-3.13 (m, 1H), 3.27-3.29 (m, 1H), 3.43-3.51 (m, 1H), 3.89-3.98 (m, 1H), 4.07-4.10 (m, 1H), 4.99-5.03 (m, 2H), 7.02-7.58 (m, 7H), 7.75-7.85 (m, 2H), 8.04-8.16 (m, 1H). Chiral separation condition: n-Hexane/EtOH contained 0.1% DEA, 70/30; S,S-WHELK-O1 (250×4.6 mm, 5 μm); retention time: 15.81 minute.

Compound 150-2: LC-MS (ESI) m/z: 618 [M+H]⁺. ¹H NMR (CD₃OD, 400 MHz): δ (ppm) 0.68-0.74 (m, 1H), 1.13-1.18 (m, 1H), 1.33-1.41 (m, 1H), 1.50-1.60 (m, 1H), 1.70-1.96 (m, 7H), 2.03-2.08 (m, 2H), 2.19-2.47 (m, 2H), 2.92-3.09 (m, 1H), 3.27-3.29 (m, 1H), 3.43-3.51 (m, 1H), 3.88-3.92 (m, 1H), 4.07-4.10 (m, 1H), 4.99-5.03 (m, 2H), 7.02-7.58 (m, 7H), 7.70-7.85 (m, 2H), 8.05-8.16 (m, 1H). Chiral separation condition: n-Hexane/EtOH contained 0.1% DEA, 70/30; S,S-WHELK-O1 (250×4.6 mm, 5 μm); retention time: 12.86 min.

### Example 151

### Synthesis of (R)—N-(1-(4′-chloro-[1,1′-biphenyl]-4-yl)-1,1-difluoro-3-oxo-3-(piperazin-1-yl)propan-2-yl)-4-(cyclohexylmethoxy)benzenesulfonamide trifluoroacetate (Compound 151-1), and (S)—N-(1-(4′-chloro-[1,1′-biphenyl]-4-yl)-1,1-difluoro-3-oxo-3-(piperazin-1-yl)propan-2-yl)-4-(cyclohexylmethoxy) benzenesulfonamide trifluoroacetate (Compound 151-2)

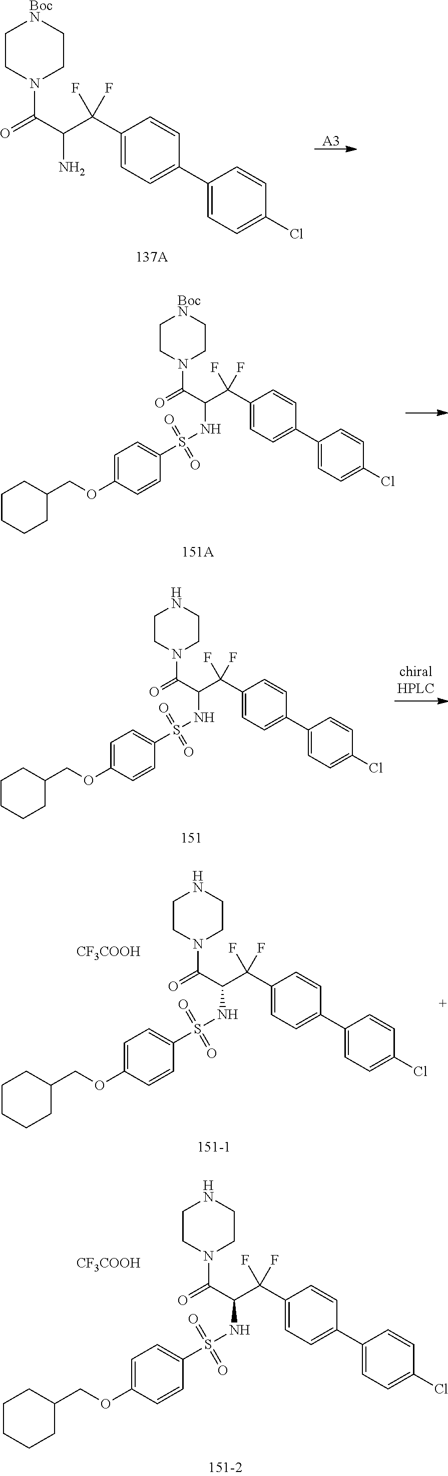

Compound 151A: LC-MS (ESI) m/z: 732 [M+H]⁺.

Compound 151 was separated with chiral HPLC to furnish Compound 151-1 and Compound 151-2.

Compound 151-1: LC-MS (ESI) m/z: 632 [M+H]⁺; ¹H-NMR (CD₃OD, 400 MHz): δ (ppm) 0.91-1.08 (m, 2H), 1.25-1.41 (m, 4H), 1.62-1.79 (m, 4H), 3.08-3.22 (m, 5H), 3.55-3.58 (m, 2H), 3.75-3.97 (m, 4H), 4.91-4.97 (m, 1H), 6.79 (d, J=8.8 Hz, 2H), 7.49-7.51 (m, 6H), 7.58 (d, J=8.4 Hz, 2H), 7.68 (d, J=8.4 Hz, 2H). Chiral separation condition: n-Hexane/EtOH contained 0.1% DEA, 50/50; S,S-Whelk-O1 (4.6×250 mm, 5 μm); retention time: 9.31 minute.

Compound 151-2: LC-MS (ESI) m/z: 632 [M+H]⁺; ¹H-NMR (CD₃OD, 400 MHz): δ (ppm) 0.92-1.06 (m, 2H), 1.25-1.41 (m, 4H), 1.62-1.79 (m, 4H), 3.08-3.22 (m, 5H), 3.55-3.58 (m, 2H), 3.75-3.97 (m, 4H), 4.91-4.97 (m, 1H), 6.79 (d, J=8.8 Hz, 2H), 7.49-7.51 (m, 6H), 7.58 (d, J=8.4 Hz, 2H), 7.66 (d, J=8.4 Hz, 2H). Chiral separation condition: n-Hexane/EtOH contained 0.1% DEA, 50/50; S,S-Whelk-O1 (4.6×250 mm, 5 μm); retention time: 8.15 minute.

### Example 152

### Synthesis of (2R)—N-((2R)-3-(3-amino-8-azabicyclo[3.2.1]octan-8-yl)-1-(4′-chloro-[1,1′-biphenyl]-4-yl)-1,1-difluoro-3-oxopropan-2-yl)-2-(4-(cyclohexylmethoxy) phenyl)-2-hydroxyacetamide trifluoroacetate (Compound 152-1), (2S)—N-((2S)-3-(3-amino-8-azabicyclo[3.2.1]octan-8-yl)-1-(4′-chloro-[1,1′-biphenyl]-4-yl)-1,1-difluoro-3-oxopropan-2-yl)-2-(4-(cyclohexylmethoxy)phenyl)-2-hydroxyacetamide trifluoroacetate (Compound 152-2), (2S)—N-((2R)-3-(3-amino-8-azabicyclo[3.2.1]octan-8-yl)-1-(4′-chloro-[1,1′-biphenyl]-4-yl)-1,1-difluoro-3-oxopropan-2-yl)-2-(4-(cyclohexylmethoxy)phenyl)-2-hydroxyacetamide trifluoroacetate (Compound 152-3), and (2R)—N-((2S)-3-(3-amino-8-azabicyclo[3.2.1]octan-8-yl)-1-(4′-chloro-[1,1′-biphenyl]-4-yl)-1,1-difluoro-3-oxopropan-2-yl)-2-(4-(cyclohexylmethoxy) phenyl)-2-hydroxyacetamide trifluoroacetate (Compound 152-4)

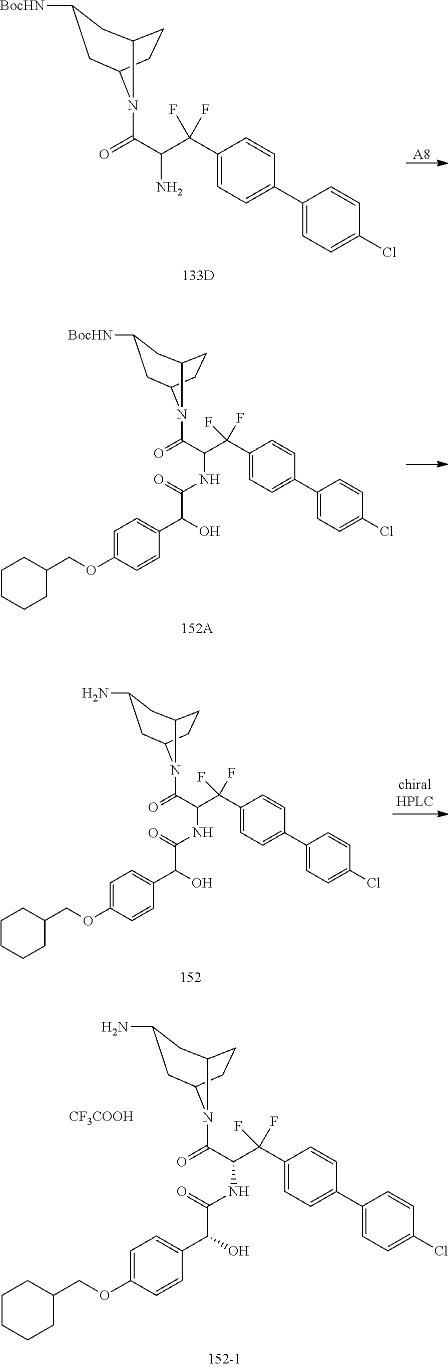 [SMILES: CC(C)(C)OC(=O)NC1CC2CCC(C1)N2C(=O)C(N)C(F)(F)c1ccc(-c2ccc(Cl)cc2)cc1 ; "A8" ; CC(C)(C)OC(=O)NC1CC2CCC(C1)N2C(=O)C(NC(=O)C(O)c1ccc(OCC2CCCCC2)cc1)C(F)(F)c1ccc(-c2ccc(Cl)cc2)cc1 ; NC1CC2CCC(C1)N2C(=O)C(NC(=O)C(O)c1ccc(OCC2CCCCC2)cc1)C(F)(F)c1ccc(-c2ccc(Cl)cc2)cc1 ; NC1CC2CCC(C1)N2C(=O)[C@@H](NC(=O)[C@H](O)c1ccc(OCC2CCCCC2)cc1)C(F)(F)c1ccc(-c2ccc(Cl)cc2)cc1 ; "CF3COOH"]

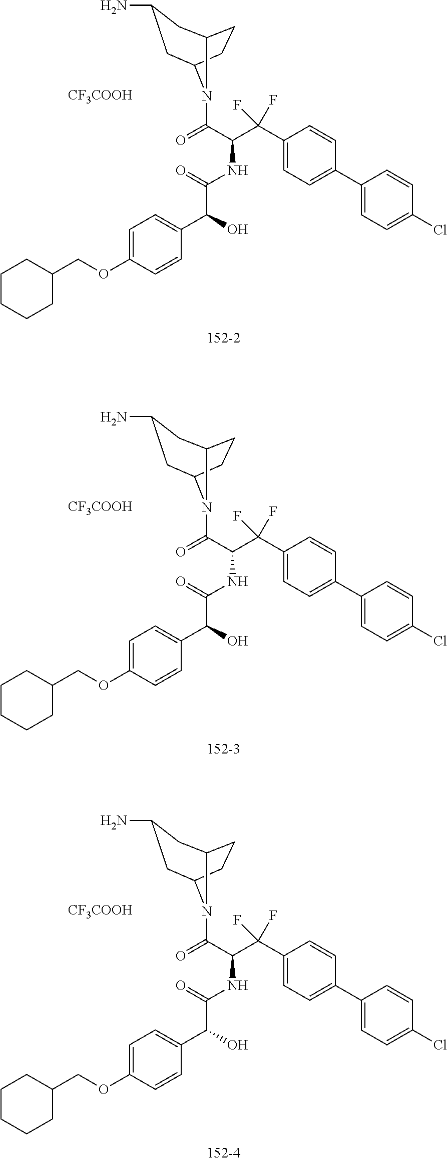 [SMILES: NC1CC2CCC(C1)N2C(=O)[C@H](NC(=O)[C@@H](O)c1ccc(OCC2CCCCC2)cc1)C(F)(F)c1ccc(-c2ccc(Cl)cc2)cc1 ; "CF3COOH" ; NC1CC2CCC(C1)N2C(=O)[C@@H](NC(=O)[C@@H](O)c1ccc(OCC2CCCCC2)cc1)C(F)(F)c1ccc(-c2ccc(Cl)cc2)cc1 ; "CF3COOH" ; NC1CC2CCC(C1)N2C(=O)[C@H](NC(=O)[C@H](O)c1ccc(OCC2CCCCC2)cc1)C(F)(F)c1ccc(-c2ccc(Cl)cc2)cc1 ; "CF3COOH"]

Compounds 152A and 152 were synthesized by employing the procedures described for Compound B4 and Compound 6 using Compound 133D using DMF as solvent and without DIPEA, Compound A8, and Compound 152A in lieu of Compounds B4-1 using dichloromethane as solvent and with DIPEA, B2-6, and Compound 6C.

Compound 152A: LC-MS (ESI) m/z: 788 [M+Na]⁺.

Compound 152 was separated with chiral HPLC to give Compound 152-1, Compound 152-2, Compound 152-3, and Compound 152-4.

Compound 152-1: LC-MS (ESI) m/z: 666 [M+H]⁺; ¹H-NMR (CD₃OD, 500 MHz): δ (ppm) 0.92-1.25 (m, 4H), 1.43-1.98 (m, 15H), 3.60-3.68 (m, 3H), 4.39-4.63 (m, 2H), 5.13-5.49 (m, 2H), 6.75-6.82 (m, 2H), 7.16-7.29 (m, 2H), 7.50-7.51 (m, 2H), 7.59-7.60 (m, 2H), 7.65-7.69 (m, 4H). Chiral separation condition: n-Hexane/EtOH contained 0.1% DEA, 50/50; S,S-Whelk-O1 (4.6×250 mm, 5 μm); retention time: 20.3 minute.

Compound 152-2: LC-MS (ESI) m/z: 666 [M+H]⁺; ¹H-NMR (CD₃OD, 500 MHz): δ (ppm) 0.92-1.24 (m, 4H), 1.43-1.98 (m, 15H), 3.60-3.68 (m, 3H), 4.39-4.63 (m, 2H), 5.13-5.49 (m, 2H), 6.75-6.82 (m, 2H), 7.16-7.29 (m, 2H), 7.50-7.51 (m, 2H), 7.59-7.60 (m, 2H), 7.65-7.69 (m, 4H). Chiral separation condition: n-Hexane/EtOH contained 0.1% DEA, 50/50; S,S-Whelk-O1 (4.6×250 mm, 5 μm); retention time: 7.45 minute.

Compound 152-3: LC-MS (ESI) m/z: 666 [M+H]⁺; ¹H-NMR (CD₃OD, 400 MHz): δ (ppm) 1.01-1.07 (m, 2H), 1.23-1.33 (m, 3H), 1.69-2.02 (m, 14H), 3.40-3.55 (m, 2H), 3.63-3.72 (m, 1H), 4.53-4.69 (m, 2H), 4.83-4.92 (m, 1H), 5.56-5.61 (m, 1H), 6.60-6.72 (m, 2H), 6.87-7.04 (m, 2H), 7.48-7.49 (m, 2H), 7.63-7.69 (m, 6H). Chiral separation condition: MeOH contained 0.5% NH₄OH; R,R-Whelk-O1 (4.6×250 mm, 5 μm); retention time: 3.41 minute.

Compound 152-4: LC-MS (ESI) m/z: 666 [M+H]⁺; ¹H-NMR (CD₃OD, 400 MHz): δ (ppm) 1.01-1.07 (m, 2H), 1.23-1.33 (m, 3H), 1.69-2.02 (m, 14H), 3.40-3.55 (m, 2H), 3.63-3.72 (m, 1H), 4.53-4.69 (m, 2H), 4.83-4.92 (m, 1H), 5.56-5.61 (m, 1H), 6.60-6.72 (m, 2H), 6.87-7.04 (m, 2H), 7.48-7.49 (m, 2H), 7.63-7.69 (m, 6H). Chiral separation condition: MeOH contained 0.5% NH₄OH; R,R-Whelk-O1 (4.6×250 mm, 5 μm); retention time: 4.78 minute.

### Example 153

### Synthesis of (R)—N-(3-(4-aminopiperidin-1-yl)-1-(4′-chloro-3′-ethoxy-[1,1′-biphenyl]-4-yl)-1,1-difluoro-3-oxopropan-2-yl)-4-(cyclohexylmethoxy)benzenesulfonamide trifluoroacetate (Compound 153-1), and (S)—N-(3-(4-aminopiperidin-1-yl)-1-(4′-chloro-3′-ethoxy-[1,1′-biphenyl]-4-yl)-1,1-difluoro-3-oxopropan-2-yl)-4-(cyclohexylmethoxy) benzenesulfonamide trifluoroacetate (Compound 153-2)

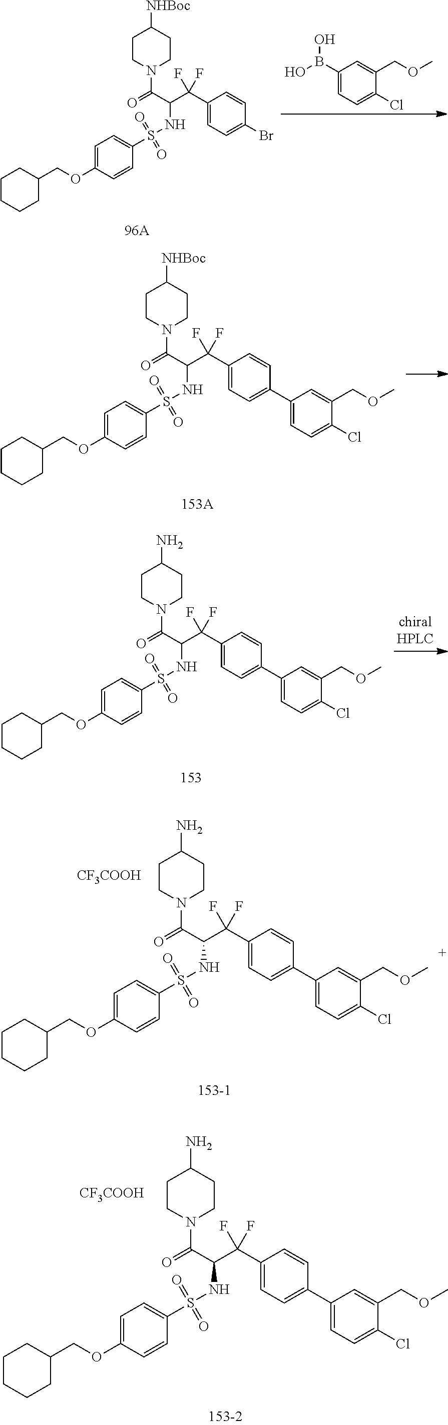

Compounds 153A and 153 were synthesized by employing the procedures described for Compounds 14C and 1 using 4-chloro-3-ethoxyphenylboronic acid, Compounds 96A, and 153A in lieu of Compounds 14B, 14A, and 1A.

Compound 153A: LC-MS (ESI) m/z: 812 [M+Na]⁺.

Compound 153 was separated with chiral HPLC to furnish Compound 153-1 and Compound 153-2.

Compound 153-1: LC-MS (ESI) m/z: 690 [M+H]⁺; ¹H-NMR (CD₃OD, 400 MHz): δ (ppm) 0.87-0.99 (m, 2H), 1.24-1.33 (m, 3H), 1.49-1.75 (m, 11H), 2.04-2.09 (m, 1H), 2.16-2.19 (m, 1H), 2.59-2.87 (m, 1H), 3.20-3.28 (m, 1H), 3.35-3.54 (m, 3H), 4.22-4.36 (m, 3H), 4.49-4.68 (m, 1H), 4.94-4.99 (m, 1H), 6.70-6.72 (m, 1H), 6.78-6.80 (m, 1H), 7.21-7.24 (m, 1H), 7.27-7.29 (m, 1H), 7.40-7.42 (m, 2H), 7.47-7.53 (m, 4H), 7.58-7.60 (m, 1H). Chiral separation condition: MeOH contained 0.5% NH₄OH; IC (4.6×250 mm, 5 μm); retention time: 7.88 minute.

Compound 153-2: LC-MS (ESI) m/z: 690 [M+H]⁺; ¹H-NMR (CD₃OD, 400 MHz): δ (ppm) 0.85-0.96 (m, 2H), 1.24-1.33 (m, 3H), 1.49-1.75 (m, 11H), 2.04-2.10 (m, 1H), 2.16-2.19 (m, 1H), 2.59-2.87 (m, 1H), 3.20-3.28 (m, 1H), 3.36-3.53 (m, 3H), 4.22-4.35 (m, 3H), 4.49-4.68 (m, 1H), 4.94-4.99 (m, 1H), 6.70-6.72 (m, 1H), 6.78-6.80 (m, 1H), 7.22-7.24 (m, 1H), 7.27-7.28 (m, 1H), 7.39-7.42 (m, 2H), 7.46-7.53 (m, 4H), 7.58-7.60 (m, 1H). Chiral separation condition: MeOH contained 0.5% NH₄OH; IC (4.6×250 mm, 5 μm); retention time: 5.71 minute.

### Example 154

### Synthesis of (R)—N-(3-(4-aminopiperidin-1-yl)-1-(4′-chloro-3′-ethoxy-[1,1′-biphenyl]-4-yl)-1,1-difluoro-3-oxopropan-2-yl)-6-(cyclopentyloxy)naphthalene-2-sulfonamide trifluoroacetate (Compound 154-1), and (S)—N-(3-(4-aminopiperidin-1-yl)-1-(4′-chloro-3′-ethoxy-[1,1′-biphenyl]-4-yl)-1,1-difluoro-3-oxopropan-2-yl)-6-(cyclopentyloxy)naphthalene-2-sulfonamide trifluoroacetate (Compound 154-2)

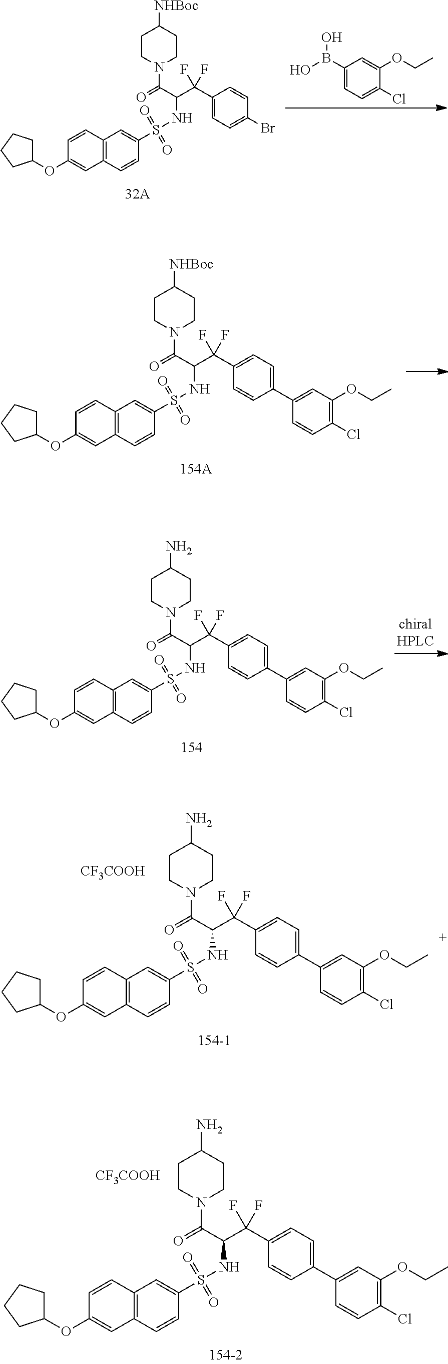

Compounds 154A and 154 were synthesized by employing the procedures described for Compounds 14C and 1 using 4-chloro-3-ethoxyphenylboronic acid, Compounds 32A and 154A in lieu of Compounds 14B, 14C, and 1A.

Compound 154A: LC-MS (ESI) m/z: 812 [M+Na]⁺.

Compound 154 was separated with chiral HPLC to furnish Compound 154-1 and Compound 154-2.

Compound 154-1: LC-MS (ESI) m/z: 712 [M+H]⁺; ¹H-NMR (CD₃OD, 400 MHz): δ (ppm) 1.30-1.42 (m, 1H), 1.50 (t, J=7.2 Hz, 3H), 1.58-2.34 (m, 11H), 2.82-3.15 (m, 1H), 3.43-4.42 (m, 4H), 4.62-4.79 (m, 2H), 4.87-4.87 (m, 1H), 4.99-5.11 (m, 1H), 6.81-7.15 (m, 4H), 7.20-7.22 (m, 1H), 7.32-7.65 (m, 6H), 7.73-7.82 (m, 1H), 8.02-8.11 (m, 1H). Chiral separation condition: MeOH contained 0.2% Methanol Ammonia; (R,R)-Whelk-O1 (4.6×250 mm, 5 μm); retention time: 10.22 minute.

Compound 154-2: LC-MS (ESI) m/z: 712 [M+H]⁺; ¹H-NMR (CD₃OD, 400 MHz): δ (ppm) 1.30-1.41 (m, 1H), 1.48 (t, J=7.2 Hz, 3H), 1.58-2.35 (m, 11H), 2.82-3.16 (m, 1H), 3.43-4.41 (m, 4H), 4.62-4.77 (m, 1.5H), 4.87-4.87 (m, 1H), 5.00-5.11 (m, 1.5H), 6.81-7.14 (m, 4H), 7.20-7.22 (m, 1H), 7.32-7.65 (m, 6H), 7.73-7.82 (m, 1H), 8.02-8.11 (m, 1H). Chiral separation condition: MeOH contained 0.2% Methanol Ammonia; (R,R)-Whelk-O1 (4.6×250 mm, 5 μm); retention time: 14.7 minute.

### Example 155

### Synthesis of N—((R)-3-((R)-3-aminopyrrolidin-1-yl)-1-(4′-chloro-[1,1′-biphenyl]-4-yl)-1,1-difluoro-3-oxopropan-2-yl)-4-(cyclohexylmethoxy)benzenesulfonamide trifluoroacetate (Compound 155-1), and N—((S)-3-((R)-3-aminopyrrolidin-1-yl)-1-(4′-chloro-[1,1′-biphenyl]-4-yl)-1,1-difluoro-3-oxopropan-2-yl)-4-(cyclohexylmethoxy) benzenesulfonamide trifluoroacetate (Compound 155-2)

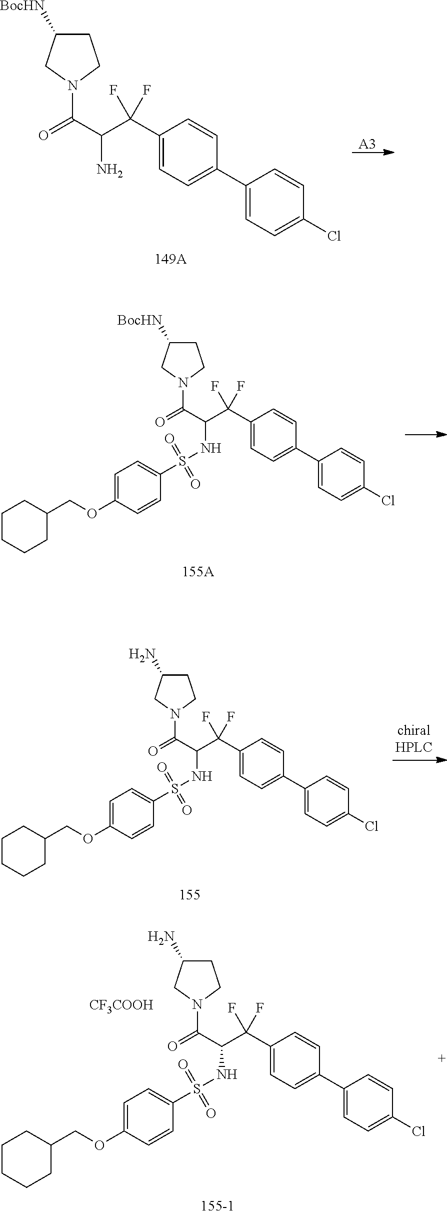 [SMILES: CC(C)(C)OC(=O)N[C@@H]1CCN(C(=O)C(N)C(F)(F)c2ccc(-c3ccc(Cl)cc3)cc2)C1 ; "A3" ; CC(C)(C)OC(=O)N[C@@H]1CCN(C(=O)C(NS(=O)(=O)c2ccc(OCC3CCCCC3)cc2)C(F)(F)c2ccc(-c3ccc(Cl)cc3)cc2)C1 ; N[C@@H]1CCN(C(=O)C(NS(=O)(=O)c2ccc(OCC3CCCCC3)cc2)C(F)(F)c2ccc(-c3ccc(Cl)cc3)cc2)C1 ; N[C@@H]1CCN(C(=O)[C@@H](NS(=O)(=O)c2ccc(OCC3CCCCC3)cc2)C(F)(F)c2ccc(-c3ccc(Cl)cc3)cc2)C1 ; "CF3COOH"]

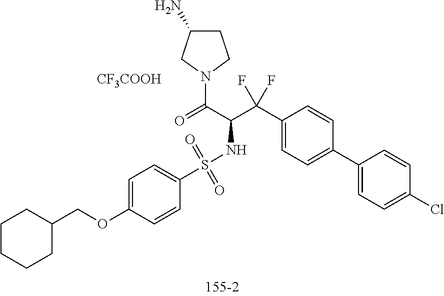 [SMILES: N[C@@H]1CCN(C(=O)[C@H](NS(=O)(=O)c2ccc(OCC3CCCCC3)cc2)C(F)(F)c2ccc(-c3ccc(Cl)cc3)cc2)C1 ; "CF3COOH"]

Compounds 155A and 155 were synthesized by employing the procedures described for Compounds 2D and 1 using Compound 149A Compound A3, and Compound 155A in lieu of Compound B2, Compounds 2C, and 1A.

Compound 155A: LC-MS (m/z): 754 [M+Na]⁺.

Compound 155 was separated with chiral HPLC to give Compound 155-1 and Compound 155-2.

Compound 155-1: LC-MS (ESI) m/z: 632 [M+H]⁺; ¹H-NMR (CD₃OD, 400 MHz): δ (ppm) 0.85-1.05 (m, 2H), 1.15-1.41 (m, 4H), 1.58-1.84 (m, 5H), 1.98-2.53 (m, 2H), 3.33-3.61 (m, 3H), 3.62-4.05 (m, 4H), 4.55-4.71 (m, 1H), 6.77 (dd, J=14.4, 8.9 Hz, 2H), 7.33-7.65 (m, 10H). Chiral separation condition: n-Hexane/EtOH contained 0.1% DEA, 50/50; IA (4.6×250 mm, 5 μm); retention time: 8.22 minute.

Compound 155-2: LC-MS (ESI) m/z: 632 [M+H]⁺; ¹H-NMR (CD₃OD, 400 MHz): δ (ppm) 0.72-0.96 (m, 2H), 1.03-1.31 (m, 4H), 1.60 (dd, J=48.2, 34.5 Hz, 5H), 2.16-2.18 (m, 2H), 3.26-3.99 (m, 7H), 4.55-4.58 (m, 1H), 6.66 (dd, J=36.9, 8.9 Hz, 2H), 7.22-7.65 (m, 10H). Chiral separation condition: n-Hexane/EtOH contained 0.1% DEA, 50/50; IA (4.6×250 mm, 5 μm); retention time: 16.88 minute.

### Example 156

### Synthesis of (R)—N-(1-([1,1′-biphenyl]-4-yl)-3-(4-aminopiperidin-1-yl)-1,1-difluoro-3-oxopropan-2-yl)-4-(cyclohexylmethoxy)benzenesulfonamide trifluoroacetate (Compound 156-1), and (S)—N-(1-([1,1′-biphenyl]-4-yl)-3-(4-aminopiperidin-1-yl)-1,1-difluoro-3-oxopropan-2-yl)-4-(cyclohexylmethoxy)benzenesulfonamide trifluoroacetate (Compound 156-2)

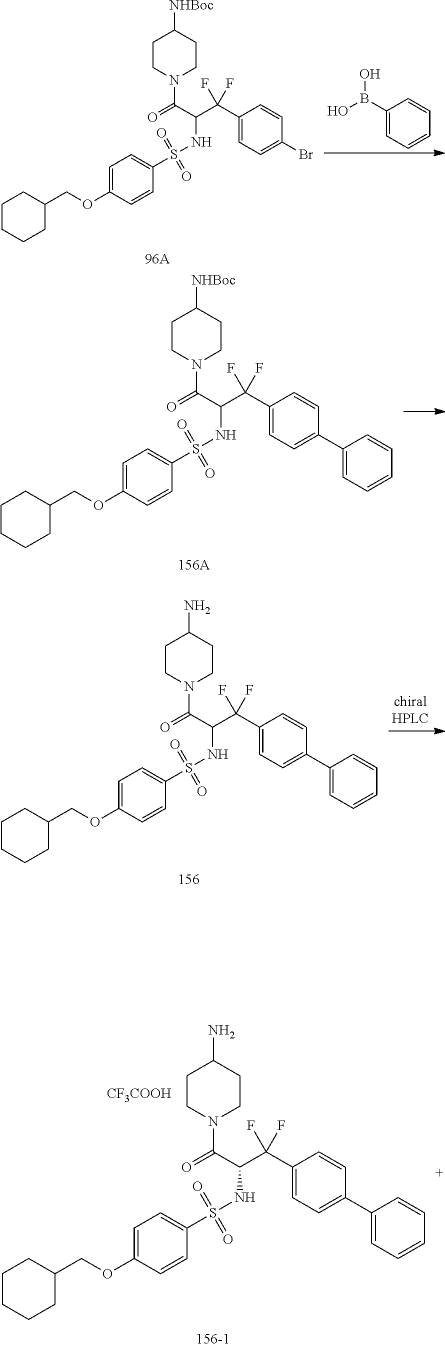 [SMILES: CC(C)(C)OC(=O)NC1CCN(C(=O)C(NS(=O)(=O)c2ccc(OCC3CCCCC3)cc2)C(F)(F)c2ccc(Br)cc2)CC1 ; OB(O)c1ccccc1 ; CC(C)(C)OC(=O)NC1CCN(C(=O)C(NS(=O)(=O)c2ccc(OCC3CCCCC3)cc2)C(F)(F)c2ccc(-c3ccccc3)cc2)CC1 ; NC1CCN(C(=O)C(NS(=O)(=O)c2ccc(OCC3CCCCC3)cc2)C(F)(F)c2ccc(-c3ccccc3)cc2)CC1 ; NC1CCN(C(=O)[C@@H](NS(=O)(=O)c2ccc(OCC3CCCCC3)cc2)C(F)(F)c2ccc(-c3ccccc3)cc2)CC1 ; "CF3COOH"]

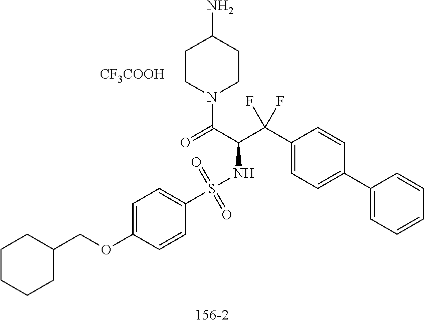 [SMILES: NC1CCN(C(=O)[C@H](NS(=O)(=O)c2ccc(OCC3CCCCC3)cc2)C(F)(F)c2ccc(-c3ccccc3)cc2)CC1 ; "CF3COOH"]

Compounds 156A and 156 were synthesized by employing the procedures described for Compounds 14C and 1 using phenylboronic acid, Compounds 96A, and 156A in lieu of Compounds 14B, 14A, and 1A.

Compound 156A: LC-MS (m/z): 734 [M+Na]⁺.

Compound 156 was separated with chiral HPLC to give Compound 156-1 and Compound 156-2.

Compound 156-1: LC-MS (ESI) m/z: 612 [M+H]⁺; ¹H-NMR (CD₃OD, 400 MHz): δ (ppm) 0.86-1.10 (m, 2H), 1.27-1.31 (m, 3H), 1.38-1.88 (m, 8H), 2.10-2.15 (m, 2H), 2.50-2.82 (m, 1H), 3.13-3.24 (m, 1H), 3.36-3.67 (m, 3H), 4.18-4.61 (m, 2H), 4.85-5.00 (m, 1H), 6.77 (d, J=8.8 Hz, 1H), 6.84 (d, J=8.8 Hz, 1H), 7.38-7.75 (m, 11H). Chiral separation condition: MeOH contained 0.5% NH₄OH; IC (4.6×150 mm, 5 μm); retention time: 6.68 minute.

Compound 156-2: LC-MS (ESI) m/z: 612 [M+H]⁺; ¹H-NMR (CD₃OD, 400 MHz): δ (ppm) 0.81-1.10 (m, 2H), 1.13-1.36 (m, 3H), 1.38-1.86 (m, 8H), 2.10-2.16 (m, 2H), 2.45-2.83 (m, 1H), 3.13-3.21 (m, 1H), 3.40-3.44 (m, 1H), 3.46-3.56 (m, 1H), 3.58-3.70 (m, 1H), 4.18-4.61 (m, 2H), 4.92-5.06 (m, 1H), 6.77 (d, J=8.8 Hz, 1H), 6.84 (d, J=8.8 Hz, 1H), 7.39-7.68 (m, 11H). Chiral separation condition: MeOH contained 0.5% NH₄OH; IC (4.6×150 mm, 5 μm); retention time: 5.02 minute.

### Example 157

### Synthesis of (R)—N-(3-aminopropyl)-3-(4′-chloro-[1,1′-biphenyl]-4-yl)-2-((6-(cyclopentyloxy)naphthalene)-2-sulfonamido)-3,3-difluoro-N-methylpropanamide (Compound 157-1), and (S)—N-(3-aminopropyl)-3-(4′-chloro-[1,1′-biphenyl]-4-yl)-2-((6-(cyclopentyloxy)naphthalene)-2-sulfonamido)-3,3-difluoro-N-methyl propanamide (Compound 157-2)

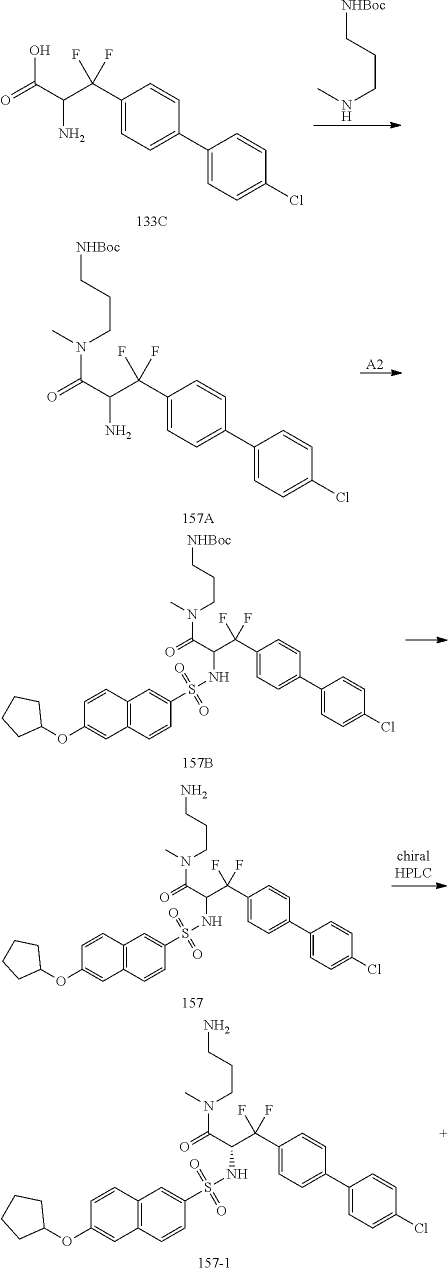

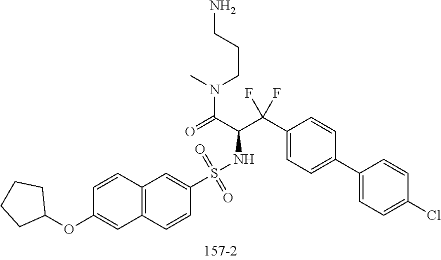 [SMILES: CN(CCCN)C(=O)[C@H](NS(=O)(=O)c1ccc2cc(OC3CCCC3)ccc2c1)C(F)(F)c1ccc(-c2ccc(Cl)cc2)cc1]

Compounds 157A, 157B, and 157 were synthesized by employing the procedures described for Compound B4, Compounds 2D, and 1 using tert-butyl 3-(methylamino)-propylcarbamate, Compounds 133C, 157A, Compound A2, and Compound 157B in lieu of Compounds B4-1, B2-6, B2, Compounds 2C, and 1A.

Compound 157A: LC-MS (ESI) m/z: 482 [M+H]⁺.

Compound 157B: LC-MS (ESI) m/z: 778 [M+Na]⁺.

Compound 157 chiral HPLC to furnish Compound 157-1 and Compound 157-2.

Compound 157-1: LC-MS (ESI) m/z: 656 [M+H]⁺; ¹H-NMR (CD₃OD, 400 MHz): δ (ppm) 1.53-1.97 (m, 12H), 2.67-2.77 (m, 2H), 3.08 (s, 3H), 3.26-3.32 (m, 1H), 4.60-4.68 (m, 1H), 6.86 (s, 1H), 6.96-7.01 (m, 1H), 7.20-7.40 (m, 9H), 7.50 (d, J=8.8 Hz, 1H), 7.67 (d, J=8.8 Hz, 1H), 7.97 (s, 1H). Chiral separation condition: MeOH contained 0.2% Methanol Ammonia; (R,R)-Whelk-O1 (4.6×250 mm, 5 μm); retention time: 6.05 minute.

Compound 157-2: LC-MS (ESI) m/z: 656.2 [M+H]⁺; ¹H-NMR (CD₃OD, 400 MHz): δ (ppm) 1.52-1.98 (m, 12H), 2.67-2.76 (m, 2H), 3.08 (s, 3H), 3.25-3.31 (m, 1H), 4.62-4.68 (m, 1H), 6.86 (d, J=2.0 Hz, 1H), 6.96-7.01 (m, 1H), 7.20-7.37 (m, 9H), 7.50 (d, J=8.8 Hz, 1H), 7.67 (d, J=8.8 Hz, 1H), 7.97 (d, J=2.0 Hz, 1H). Chiral separation condition: MeOH contained 0.2% Methanol Ammonia; (R,R)-Whelk-O1 (4.6×250 mm, 5 μm); retention time: 8.41 minute.

### Example 158

### Synthesis of (2R)—N-((2R)-3-(3-amino-8-azabicyclo[3.2.1]octan-8-yl)-1-(6-bromonaphthalen-2-yl)-1,1-difluoro-3-oxopropan-2-yl)-2-(6-(cyclopentyloxy) naphthalen-2-yl)-2-hydroxyacetamide trifluoroacetate (Compound 158-1), (2S)—N-((2 S)-3-(3-amino-8-azabicyclo[3.2.1]octan-8-yl)-1-(6-bromonaphthalen-2-yl)-1,1-difluoro-3-oxopropan-2-yl)-2-(6-(cyclopentyloxy)naphthalen-2-yl)-2-hydroxyacetamide trifluoroacetate (Compound 158-2), (2S)—N-((2R)-3-(3-amino-8-azabicyclo[3.2.1]octan-8-yl)-1-(6-bromonaphthalen-2-yl)-1,1-difluoro-3-oxopropan-2-yl)-2-(6-(cyclopentyloxy) naphthalen-2-yl)-2-hydroxyacetamide trifluoroacetate (Compound 158-3), and (2R)—N-((2S)-3-(3-amino-8-azabicyclo[3.2.1]octan-8-yl)-1-(6-bromonaphthalen-2-yl)-1,1-difluoro-3-oxopropan-2-yl)-2-(6-(cyclopentyloxy) naphthalen-2-yl)-2-hydroxyacetamide trifluoroacetate (Compound 158-4)

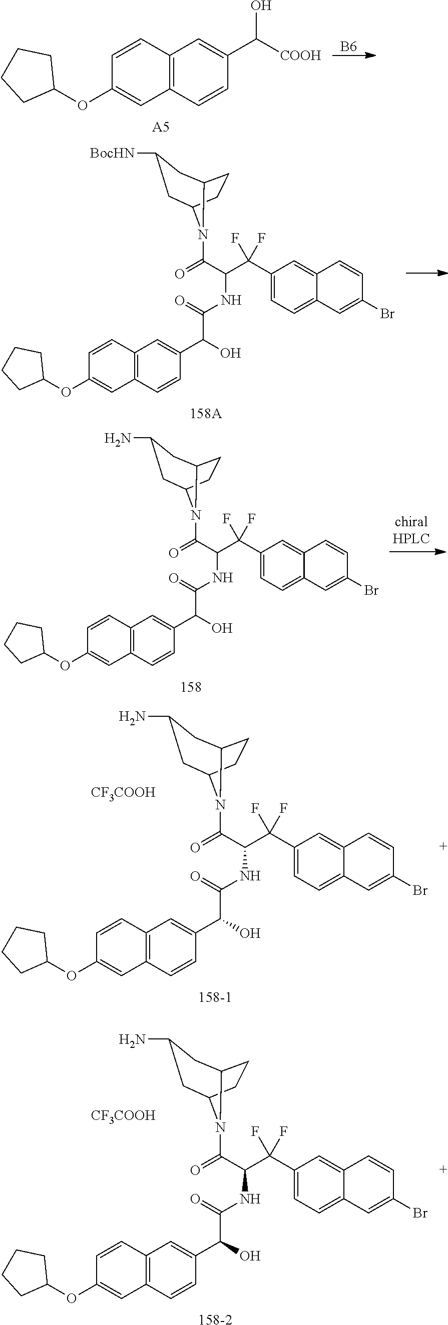

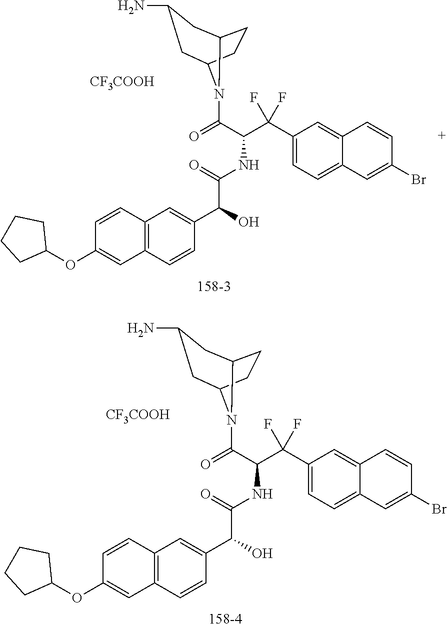

Compounds 158A and 158 were synthesized by employing the procedures described for Compound B4 and Compound 1 using Compounds B6, A5, and Compound 158A in lieu of Compounds B4-1, B2-6, and Compound 1A.

Compound 158A, which was used directly in the next step without further purification. LC-MS (ESI) m/z: 806 [M+H]⁺.

Compound 158 was separated with chiral HPLC to furnish Compound 158-1, Compound 158-2, Compound 158-3, and Compound 158-4.

Compound 158-1: LC-MS (ESI) m/z: 706 [M+H]⁺; ¹H-NMR (CD₃OD, 400 MHz): δ (ppm) 1.08-1.15 (m, 1H), 1.59-2.04 (m, 15H), 3.54-3.57 (m, 1H), 4.37-4.38 (m, 1H), 4.60-4.61 (m, 1H), 4.97-4.98 (m, 1H), 5.25 (s, 1H), 5.54-5.60 (m, 1H), 7.07-7.13 (m, 2H), 7.37-7.88 (m, 8H), 7.98-8.07 (m, 2H). Chiral separation condition: n-Hexane/EtOH contained 0.1% DEA, 85/15; IA (4.6×250 mm, 5 μm); retention time: 5.37 minute.

Compound 158-2: LC-MS (ESI) m/z: 706 [M+H]⁺; ¹H-NMR (CD₃OD, 400 MHz): δ (ppm) 1.08-1.15 (m, 1H), 1.60-2.04 (m, 15H), 3.53-3.57 (m, 1H), 4.37-4.38 (m, 1H), 4.61-4.62 (m, 1H), 4.97-4.98 (m, 1H), 5.25 (s, 1H), 5.54-5.64 (m, 1H), 7.08-7.13 (m, 2H), 7.37-7.76 (m, 8H), 7.01-8.07 (m, 2H). Chiral separation condition: n-Hexane/EtOH contained 0.1% DEA, 85/15; IA (4.6×250 mm, 5 μm); retention time: 9.35 minute.

Compound 158-3: LC-MS (ESI) m/z: 706 [M+H]⁺; ¹H-NMR (CD₃OD, 400 MHz): δ (ppm) 1.30-1.41 (m, 1H), 1.69-2.08 (m, 15H), 3.64-3.74 (m, 1H), 4.50-4.51 (m, 1H), 4.67-4.68 (m, 1H), 4.98-4.99 (m, 1H), 5.10 (s, 1H), 5.62-5.70 (m, 1H), 6.89-7.73 (m, 10H), 7.88-8.03 (m, 2H). Chiral separation condition: MeOH contained 0.2% Methanol Ammonia; (R, R)-Whelk-O1 (4.6×250 mm, 5 μm); retention time: 6.1 minute.

Compound 158-4: LC-MS (ESI) m/z: 706 [M+H]⁺; ¹H-NMR (CD₃OD, 400 MHz): δ (ppm) 1.30-1.39 (m, 1H), 1.69-2.06 (m, 15H), 3.63-3.71 (m, 1H), 4.50-4.51 (m, 1H), 4.67-4.68 (m, 1H), 4.98-4.99 (m, 1H), 5.10 (s, 1H), 5.62-5.71 (m, 1H), 7.04-7.14 (m, 3H), 7.35-7.73 (m, 7H), 7.88-8.03 (m, 2H). Chiral separation condition: MeOH contained 0.2% Methanol Ammonia; (R, R)-Whelk-O1 (4.6×250 mm, 5 μm); retention time: 13.6 minute.

### Example 159

### Synthesis of (R)—N—((R)-3-(4-aminopiperidin-1-yl)-1-(4′-chloro-[1,1′-biphenyl]-4-yl)-1,1-difluoro-3-oxopropan-2-yl)-2-(6-(cyclopentyloxy)naphthalen-2-yl)-2-hydroxyacetamide trifluoroacetate (Compound 159-1), (S)—N—((S)-3-(4-aminopiperidin-1-yl)-1-(4′-chloro-[1,1′-biphenyl]-4-yl)-1,1-difluoro-3-oxopropan-2-yl)-2-(6-(cyclopentyloxy) naphthalen-2-yl)-2-hydroxyacetamide trifluoroacetate (Compound 159-2), (S)—N—((R)-3-(4-aminopiperidin-1-yl)-1-(4′-chloro-[1,1′-biphenyl]-4-yl)-1,1-difluoro-3-oxopropan-2-yl)-2-(6-(cyclopentyloxy)naphthalen-2-yl)-2-hydroxyacetamide trifluoroacetate (Compound 159-3), and (R)—N—((S)-3-(4-aminopiperidin-1-yl)-1-(4′-chloro-[1,1′-biphenyl]-4-yl)-1,1-difluoro-3-oxopropan-2-yl)-2-(6-(cyclopentyloxy)naphthalen-2-yl)-2-hydroxyacetamide trifluoroacetate (Compound 159-4)

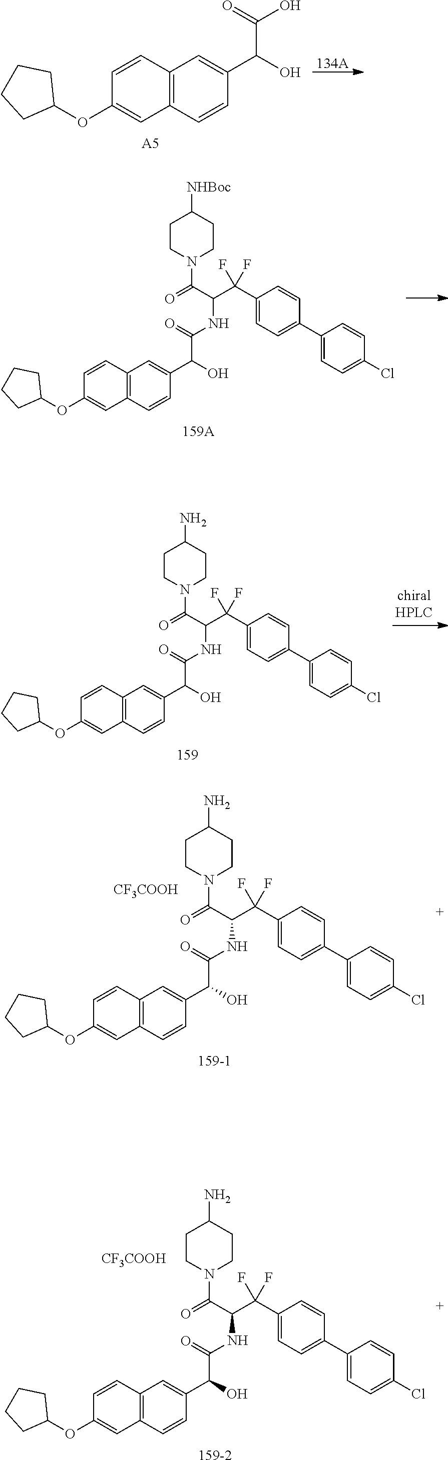

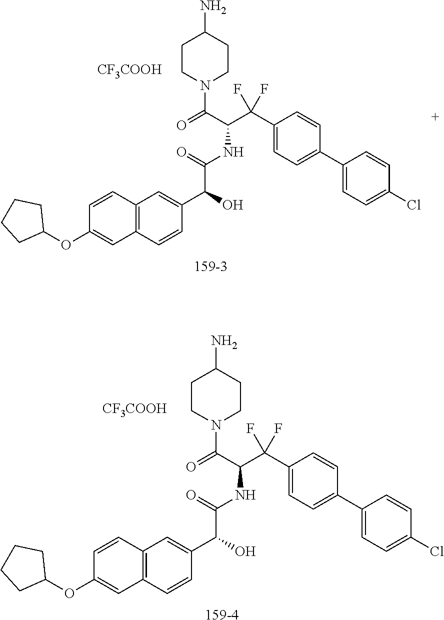

Compounds 159A and 159 were synthesized by employing the procedures described for Compound B4 and Compound 6 using Compound 134A, Compound A5 using DMF as solvent, and Compound 159A in lieu of Compounds B4-1, B2-6 using dichloromethane as solvent, and Compound 6C.

Compound 159A: LC-MS (ESI) m/z: 762[M+H]⁺.

Compound 159 was separated with chiral HPLC to afford Compound 159-1, Compound 159-2, Compound 159-3, and Compound 159-4.

Compound 159-1: LC-MS (ESI) m/z: 662 [M+H]⁺. ¹H-NMR (CD₃OD, 400 MHz): δ (ppm) 1.52-2.13 (m, 12H), 2.75-2.85 (m, 1H), 3.06-3.16 (m, 1H), 3.37-3.48 (m, 1H), 4.23-4.31 (m, 1H), 4.65-4.68 (m, 1H), 4.94-4.97 (m, 1H), 5.17-5.22 (m, 1H), 5.72-5.78 (m, 1H), 7.05-7.08 (m, 2H), 7.30-7.46 (m, 9H), 7.62-7.76 (m, 3H). Chiral separation condition: MeOH contained 0.2% Methanol Ammonia; OJ-H (250×4.6 mm, 5 μm); retention time: 3.76 minute.

Compound 159-2: LC-MS (ESI) m/z: 662 [M+H]⁺. ¹H-NMR (CD₃OD, 400 MHz): δ (ppm) 1.43-2.12 (m, 12H), 2.76-2.84 (m, 1H), 3.04-3.13 (m, 1H), 3.39-3.47 (m, 1H), 4.22-4.31 (m, 1H), 4.64-4.68 (m, 1H), 4.94-4.97 (m, 1H), 5.16-5.22 (m, 1H), 5.73-5.79 (m, 1H), 7.04-7.07 (m, 2H), 7.31-7.46 (m, 9H), 7.62-7.76 (m, 3H). Chiral separation condition: MeOH contained 0.2% Methanol Ammonia; OJ-H (250×4.6 mm, 5 μm); retention time: 6.02 minute.

Compound 159-3: LC-MS (ESI) m/z: 662 [M+H]⁺. ¹H-NMR (CD₃OD, 400 MHz): δ (ppm) 1.72-2.19 (m, 12H), 2.84-2.91 (m, 1H), 3.13-3.26 (m, 1H), 3.40-3.48 (m, 1H), 4.32-4.38 (m, 1H), 4.69-4.77 (m, 1H), 4.93-4.95 (m, 1H), 5.04-5.07 (m, 1H), 5.69-5.77 (m, 1H), 7.02-7.05 (m, 2H), 7.13-7.15 (m, 2H), 7.19-7.29 (m, 3H), 7.35-7.43 (m, 4H), 7.53-7.58 (m, 1H), 7.65-7.68 (m, 2H). Chiral separation condition: n-Hexane/EtOH contained 0.1% DEA, 50/50; OZ-H (250×4.6 mm, 5 μm); retention time: 6.64 minute.

Compound 159-4: LC-MS (ESI) m/z: 662 [M+H]⁺. ¹H-NMR (CD₃OD, 400 MHz): δ (ppm) 1.71-2.19 (m, 12H), 2.84-2.91 (m, 1H), 3.26 (s, 1H), 3.39-3.49 (m, 1H), 4.32-4.39 (m, 1H), 4.71-4.77 (m, 1H), 4.93-4.95 (m, 1H), 5.04-5.07 (m, 1H), 5.69-5.77 (m, 1H), 7.02-7.05 (m, 2H), 7.13-7.15 (m, 2H), 7.19-7.29 (m, 3H), 7.35-7.43 (m, 4H), 7.53-7.58 (m, 1H), 7.65-7.68 (m, 2H). Chiral separation condition: n-Hexane/EtOH contained 0.1% DEA, 50/50; OZ-H (250×4.6 mm, 5 μm); retention time: 11.07 minute.

### Example 160

### Synthesis of N-((2R)-3-(3-amino-8-azabicyclo[3.2.1]octan-8-yl)-1-(6-bromonaphthalen-2-yl)-1,1-difluoro-3-oxopropan-2-yl)-2-(6-(cyclopentyloxy) naphthalen-2-yl)-2-oxoacetamide trifluoroacetate (Compound 160-1), and N-((2S)-3-(3-amino-8-azabicyclo[3.2.1]octan-8-yl)-1-(6-bromonaphthalen-2-yl)-1,1-difluoro-3-oxopropan-2-yl)-2-(6-(cyclopentyloxy)naphthalen-2-yl)-2-oxoacetamide trifluoroacetate (Compound 160-2)

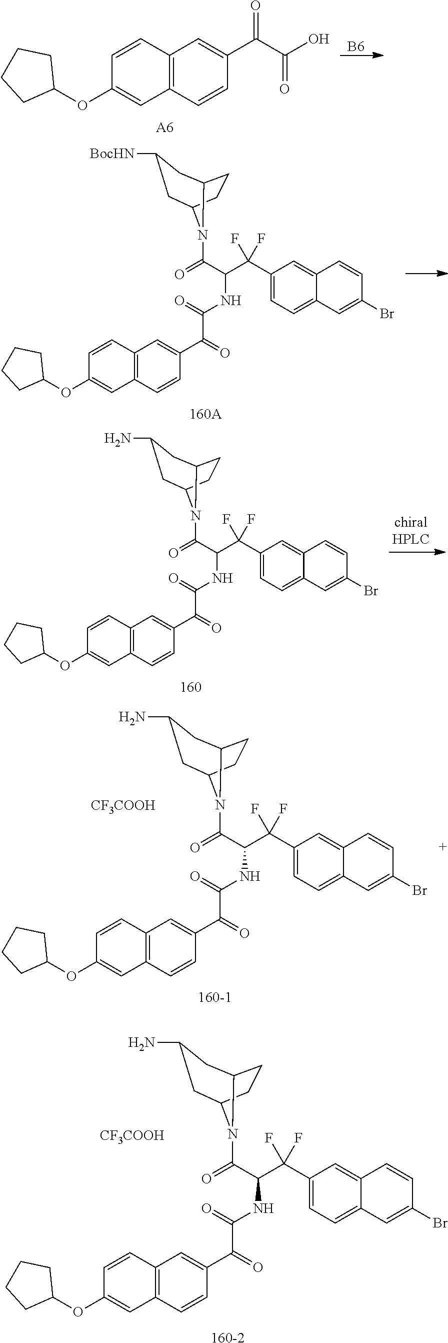

Compounds 160A and 160 were synthesized by employing the procedures described for Compound B4 and Compound 1 using Compounds B6, A6, and Compound 160A in lieu of Compounds B4-1, B2-6, and Compound 1A.

Compound 160A was used directly in the next step without further purification. LC-MS (ESI) m/z: 804 [M+H]⁺.

Compound 160 was separated with chiral HPLC to furnish Compound 160-1 and Compound 160-2.

Compound 160-1: LC-MS (ESI) m/z: 704 [M+H]⁺; ¹H-NMR (DMSO-d₆, 400 MHz): δ (ppm) 1.64-2.03 (m, 16H), 3.51-3.61 (m, 1H), 4.58-4.99 (m, 3H), 5.87-5.96 (m, 1H), 7.02-7.19 (m, 2H), 7.28-7.31 (m, 1H), 7.50-7.75 (m, 4H), 7.92-8.10 (m, 6H), 8.29-8.30 (m, 1H), 8.40-8.41 (m, 1H), 9.84-9.96 (m, 1H). Chiral separation condition: n-Hexane/EtOH contained 0.1% DEA, 30/70; OZ-H (4.6×250 mm, 5 μm); retention time: 13.51 minute.

Compound 160-2: LC-MS (ESI) m/z: 704 [M+H]⁺; ¹H-NMR (DMSO-d₆, 400 MHz): δ (ppm) 1.64-2.02 (m, 16H), 3.74-3.95 (m, 1H), 4.58-4.98 (m, 3H), 5.87-5.96 (m, 1H), 6.98-7.19 (m, 2H), 7.31-7.41 (m, 1H), 7.50-7.73 (m, 4H), 7.92-8.11 (m, 6H), 8.29-8.30 (m, 1H), 8.40-8.41 (m, 1H), 9.85-9.97 (m, 1H). Chiral separation condition: n-Hexane/EtOH contained 0.1% DEA, 30/70; OZ-H (4.6×250 mm, 5 μm); retention time: 6.08 minute.

### Example 161

### Synthesis of N—((R)-3-((R)-4-amino-3,3-difluoropiperidin-1-yl)-1-(4′-chloro-[1,1′-biphenyl]-4-yl)-1,1-difluoro-3-oxopropan-2-yl)-6-(cyclopentyloxy)naphthalene-2-sulfonamide trifluoroacetate (Compound 161-1), N—((S)-3-((S)-4-amino-3,3-difluoropiperidin-1-yl)-1-(4′-chloro-[1,1′-biphenyl]-4-yl)-1,1-difluoro-3-oxopropan-2-yl)-6-(cyclopentyloxy) naphthalene-2-sulfonamide trifluoroacetate (Compound 161-2), N—((R)-3-((S)-4-amino-3,3-difluoropiperidin-1-yl)-1-(4′-chloro-[1,1′-biphenyl]-4-yl)-1,1-difluoro-3-oxopropan-2-yl)-6-(cyclopentyloxy)naphthalene-2-sulfonamide trifluoroacetate (Compound 161-3), and N—((S)-3-((R)-4-amino-3,3-difluoropiperidin-1-yl)-1-(4′-chloro-[1,1′-biphenyl]-4-yl)-1,1-difluoro-3-oxopropan-2-yl)-6-(cyclopentyloxy)naphthalene-2-sulfonamide trifluoroacetate (Compound 161-4)

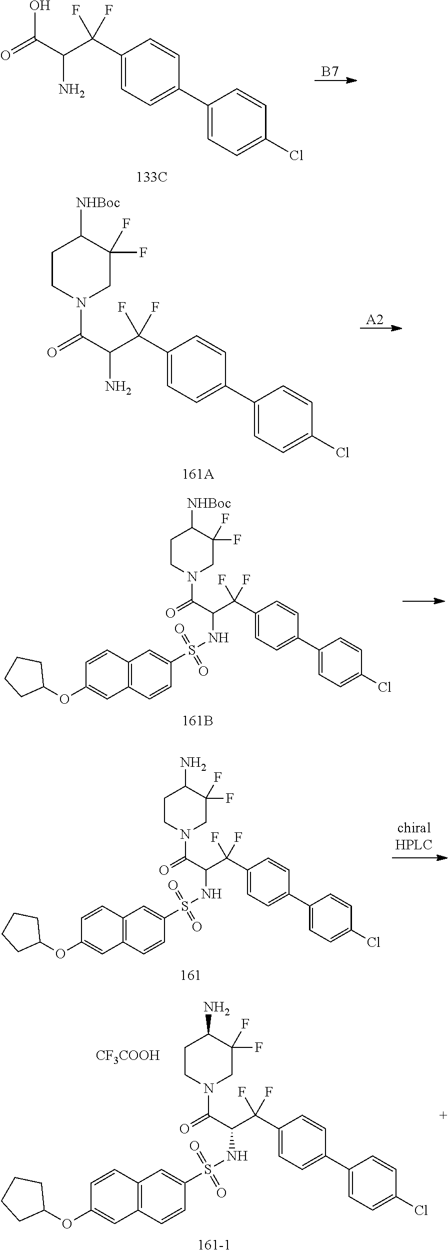

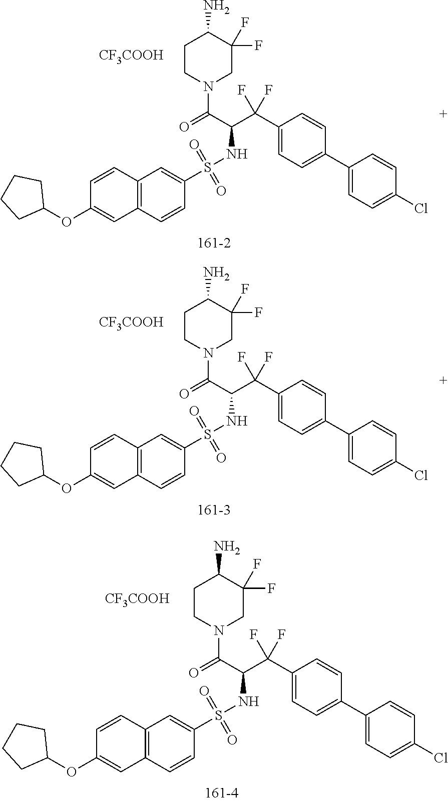 [SMILES: N[C@H]1CCN(C(=O)[C@H](NS(=O)(=O)c2ccc3cc(OC4CCCC4)ccc3c2)C(F)(F)c2ccc(-c3ccc(Cl)cc3)cc2)CC1(F)F ; "CF3COOH" ; N[C@H]1CCN(C(=O)[C@@H](NS(=O)(=O)c2ccc3cc(OC4CCCC4)ccc3c2)C(F)(F)c2ccc(-c3ccc(Cl)cc3)cc2)CC1(F)F ; "CF3COOH" ; N[C@@H]1CCN(C(=O)[C@H](NS(=O)(=O)c2ccc3cc(OC4CCCC4)ccc3c2)C(F)(F)c2ccc(-c3ccc(Cl)cc3)cc2)CC1(F)F ; "CF3COOH"]

Compounds 161A, 161B, and 161 were synthesized by employing the procedures described for Compound B4, Compounds 2D, and 1 using Compound B7, Compounds 133C, 161A, Compound A2, and Compound 161B in lieu of Compounds B4-1, B2-6, B2, Compounds 2C, and 1A.

Compound 161A: LC-MS (ESI) m/z: 530 [M+H]⁺.

Compound 161B: LC-MS (ESI) m/z: 748 [M−55]⁺.

Compound 161 was separated with chiral HPLC to furnish Compound 161-1, Compound 161-2, Compound 161-3, and Compound 161-4.

Compound 161-1: LC-MS (ESI) m/z: 704 [M+H]⁺; ¹H-NMR (CD₃OD, 400 MHz): δ (ppm) 1.70-2.00 (m, 6H), 2.09-2.36 (m, 4H), 3.15-3.27 (m, 1H), 3.41-3.64 (m, 1H), 3.93-4.04 (m, 1H), 4.50-4.79 (m, 2H), 4.99-5.18 (m, 2H), 6.89-7.80 (m, 13H), 7.99-8.10 (m, 1H). Chiral separation condition: n-Hexane/EtOH contained 0.1% DEA, 50/50; IC (4.6×250 mm, 5 μm); retention time: 10.89 minute.

Compound 161-2: LC-MS (ESI) m/z: 704 [M+H]⁺; ¹H-NMR (CD₃OD, 400 MHz): δ (ppm) 1.70-1.85 (m, 7H), 2.00-2.28 (m, 3H), 2.69-2.99 (m, 1H), 3.11-3.25 (m, 1H), 3.46-4.00 (m, 1H), 4.39-4.72 (m, 3H), 4.93-5.25 (m, 1H), 6.92-7.81 (m, 13H), 8.04-8.10 (m, 1H). Chiral separation condition: n-Hexane/EtOH contained 0.1% DEA, 50/50; IC (4.6×250 mm, 5 μm); retention time: 13.08 minute.

Compound 161-3: LC-MS (ESI) m/z: 704 [M+H]⁺; ¹H-NMR (CD₃OD, 400 MHz): δ (ppm) 1.72-2.30 (m, 10H), 3.19-3.28 (m, 1H), 3.43-3.49 (m, 1H), 3.87-4.03 (m, 1H), 4.50-4.75 (m, 2H), 5.00-5.18 (m, 2H), 6.91-7.75 (m, 13H), 8.01-8.11 (m, 1H). Chiral separation condition: n-Hexane/EtOH contained 0.1% DEA, 50/50; (R, R)-Whelk-O1 (4.6×250 mm, 5 μm); retention time: 11.40 minute.

Compound 161-4: LC-MS (ESI) m/z: 704 [M+H]⁺; ¹H-NMR (CD₃OD, 400 MHz): δ (ppm) 1.70-2.27 (m, 10H), 2.71-2.80 (m, 1H), 3.19-3.26 (m, 1H), 3.64-3.94 (m, 1H), 4.40-4.75 (m, 3H), 5.08-5.26 (m, 1H), 6.95-7.82 (m, 13H), 8.04-8.11 (m, 1H). Chiral separation condition: n-Hexane/EtOH contained 0.1% DEA, 50/50; (R, R)-Whelk-O1 (4.6×250 mm, 5 μm); retention time: 13.28 minute.

### Example 162

### Synthesis of (R)—N-(3-(4-aminopiperidin-1-yl)-1-(5-(4-chlorophenyl)pyridin-2-yl)-1,1-difluoro-3-oxopropan-2-yl)-6-(cyclopentyloxy)naphthalene-2-sulfonamide (Compound 162-1), and (S)—N-(3-(4-aminopiperidin-1-yl)-1-(5-(4-chlorophenyl) pyridin-2-yl)-1,1-difluoro-3-oxopropan-2-yl)-6-(cyclopentyloxy)naphthalene-2-sulfonamide (Compound 162-2)

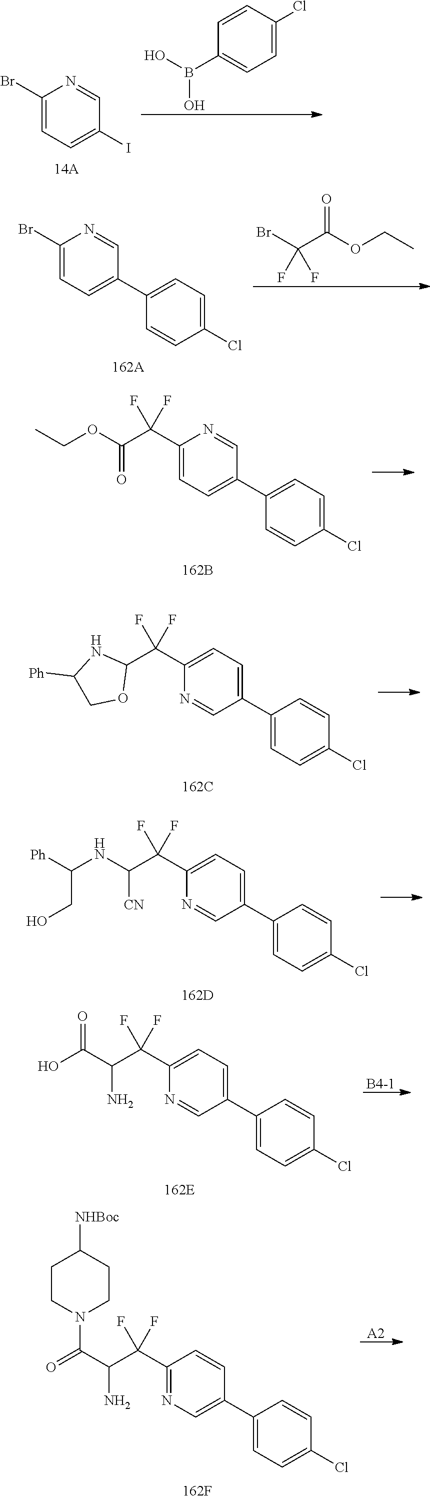

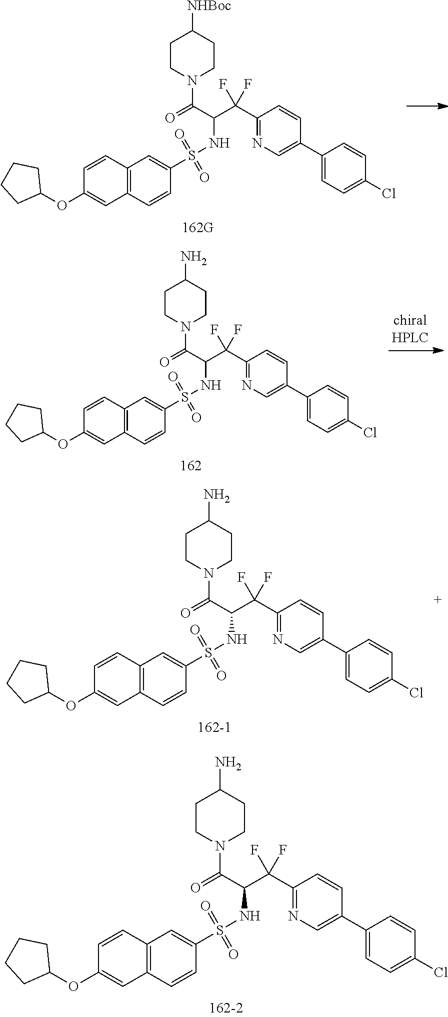 [SMILES: CC(C)(C)OC(=O)NC1CCN(C(=O)C(NS(=O)(=O)c2ccc3cc(OC4CCCC4)ccc3c2)C(F)(F)c2ccc(-c3ccc(Cl)cc3)cn2)CC1 ; NC1CCN(C(=O)C(NS(=O)(=O)c2ccc3cc(OC4CCCC4)ccc3c2)C(F)(F)c2ccc(-c3ccc(Cl)cc3)cn2)CC1 ; NC1CCN(C(=O)[C@@H](NS(=O)(=O)c2ccc3cc(OC4CCCC4)ccc3c2)C(F)(F)c2ccc(-c3ccc(Cl)cc3)cn2)CC1 ; NC1CCN(C(=O)[C@H](NS(=O)(=O)c2ccc3cc(OC4CCCC4)ccc3c2)C(F)(F)c2ccc(-c3ccc(Cl)cc3)cn2)CC1]

Compound 162A was synthesized by employing the procedure described for Compound 14C using 4-chlorophenylboronic acid in lieu of Compound 14B. LC-MS (ESI) m/z: 268 [M+H]⁺.

To a mixture of Compound 162A (2 g, 7.48 mmol) and ethyl 2-bromo-2,2-difluoroacetate (1.63 g, 8.22 mmol) in DMF (20 mL) at room temperature was added copper powder (1.08 g, 16.46 mmol) and stirred at 50° C. under nitrogen overnight. To the reaction mixture was added a solution of potassium dihydrogen phosphate (2.2 g, 16.46 mmol) in H₂O (50 mL), while the temperature was kept below 30° C. After stirred for 30 minutes, the reaction mixture was filtered to remove the precipitated copper salts and the solid was washed with ethyl acetate (50 mL×2). The filtrate was extracted with ethyl acetate (100 mL×3). The combined organic extracts was washed with brine (200 mL), dried over anhydrous sodium sulfate, filtered, and concentrated under reduced pressure. The residue was purified by silica gel column chromatography to furnish Compound 162B. ¹H-NMR (CDCl₃, 400 MHz): δ (ppm) 1.35 (t, J=6.8 Hz, 3H), 4.40 (q, J=6.8 Hz, 2H), 7.47-7.54 (m, 4H), 7.82 (dd, J=0.8, 7.6 Hz, 1H), 8.00 (dd, J=2.4, 8.0 Hz, 1H), 8.83 (d, J=1.6 Hz, 1H).

Compounds 162C, 162D, 162E, 162F, 162G, and 162 were synthesized by employing the procedures described for Compounds B2-4, B2-5, B2-6, B4, Compounds 1A, and 6 using Compounds 162B, 162C, 162D, 162E, 162F, Compound A2, and Compound 162G in lieu of Compounds B2-3, B2-4, B2-5, B2-6, B2, A1, and Compound 6C.

Compound 162C: LC-MS (ESI) m/z: 387 [M+H]⁺.

Compound 162D: LC-MS (ESI) m/z: 414 [M+H]⁺.

Compound 162E: LC-MS (ESI) m/z: 313 [M+H]⁺.

Compound 162F: LC-MS (ESI) m/z: 495 [M+H]⁺.

Compound 162G was used directly for next step without further purification. LC-MS (ESI) m/z: 769 [M+H]⁺.

Compound 162 was further puriseparated with chiral HPLC to give Compound 162-1 and 162-2.

Compound 162-1: LC-MS (m/z): 669 [M+1]⁺; ¹H-NMR (CD₃OD, 400 MHz): δ (ppm) 1.56-1.90 (m, 7H), 1.98-2.26 (m, 4H), 2.55-2.93 (m, 1H), 3.36-3.49 (m, 3H), 4.51-4.73 (m, 3H), 5.48-5.62 (m, 1H), 6.79-6.80 (m, 1H), 7.05-7.12 (m, 1H), 7.27-7.66 (m, 7H), 7.74-8.00 (m, 3H), 8.06-8.19 (m, 1H). Chiral separation condition: MeOH (0.2% NH₄OH); (R,R)-Whelk-O1 (4.6×250 mm, 5 μm); retention time: 5.54 minute.

Compound 162-2: LC-MS (m/z): 669 [M+1]⁺; ¹H-NMR (CD₃OD, 400 MHz): δ (ppm) 1.52-1.96 (m, 10H), 2.35-2.63 (m, 1H), 2.66-2.95 (m, 2H), 3.00-3.09 (m, 1H), 3.49-3.79 (m, 1H), 4.03-4.28 (m, 2H), 4.49-4.65 (m, 1H), 5.31-5.40 (m, 1H), 6.76-7.09 (m, 3H), 7.27-7.37 (m, 4H), 7.46-7.55 (m, 2H), 7.68-7.72 (m, 1H), 7.80-7.84 (m, 1H), 7.98-8.00 (m, 1H), 8.15 (s, 1H). Chiral separation condition: MeOH (0.2% NH₄OH); (R,R)-Whelk-O1 (4.6×250 mm, 5 μm); retention time: 10.28 minute.

### Example 163

### Synthesis of (R)—N-(3-(4-aminopiperidin-1-yl)-1-(5-(4-chlorophenyl)pyridin-2-yl)-1,1-difluoro-3-oxopropan-2-yl)-4-(cyclohexylmethoxy)benzenesulfonamide (Compound 163-1), and (S)—N-(3-(4-aminopiperidin-1-yl)-1-(5-(4-chlorophenyl) pyridin-2-yl)-1,1-difluoro-3-oxopropan-2-yl)-4-(cyclohexylmethoxy) benzenesulfonamide (Compound 163-2)

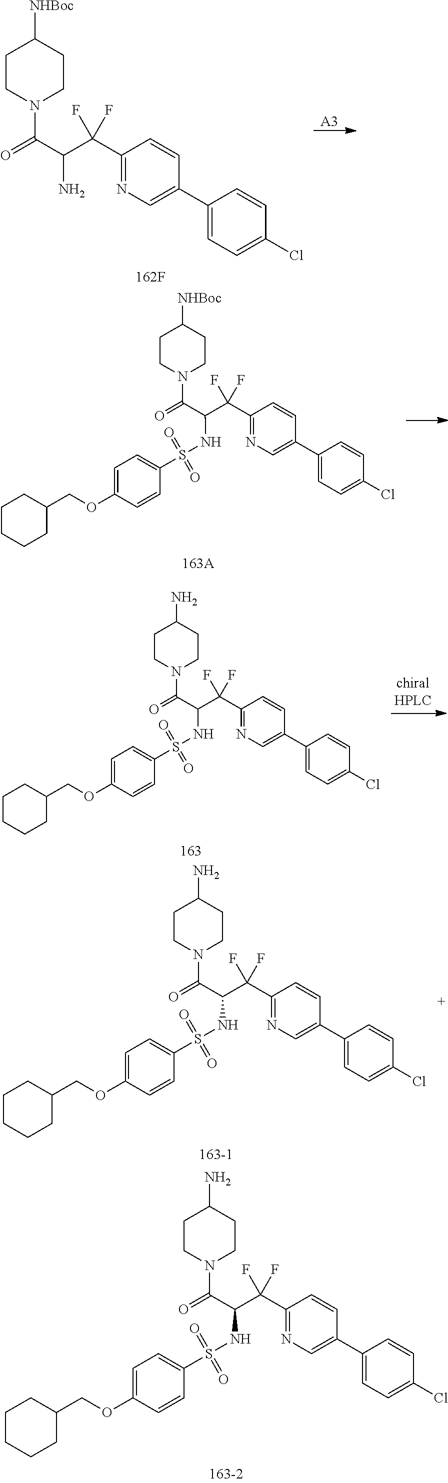 [SMILES: CC(C)(C)OC(=O)NC1CCN(C(=O)C(N)C(F)(F)c2ccc(-c3ccc(Cl)cc3)cn2)CC1 ; "A3" ; CC(C)(C)OC(=O)NC1CCN(C(=O)C(NS(=O)(=O)c2ccc(OCC3CCCCC3)cc2)C(F)(F)c2ccc(-c3ccc(Cl)cc3)cn2)CC1 ; NC1CCN(C(=O)C(NS(=O)(=O)c2ccc(OCC3CCCCC3)cc2)C(F)(F)c2ccc(-c3ccc(Cl)cc3)cn2)CC1 ; NC1CCN(C(=O)[C@@H](NS(=O)(=O)c2ccc(OCC3CCCCC3)cc2)C(F)(F)c2ccc(-c3ccc(Cl)cc3)cn2)CC1 ; NC1CCN(C(=O)[C@H](NS(=O)(=O)c2ccc(OCC3CCCCC3)cc2)C(F)(F)c2ccc(-c3ccc(Cl)cc3)cn2)CC1]

Compounds 163A and 163 were synthesized by employing the procedures described for Compounds 1A and 6 using Compound 162F, Compound A3, and Compound 163A in lieu of Compounds B2, A1, and Compound 6C.

Compound 163A was used directly for next step without further purification. LC-MS (ESI) m/z: 747 [M+H]⁺.

Compound 163 was separated with chiral HPLC to give Compound 163-1 and Compound 163-2.

Compound 163-1: LC-MS (m/z): 647 [M+1]⁺; ¹H-NMR (CD₃OD, 400 MHz): δ (ppm) 0.89-2.18 (m, 15H), 2.68-2.92 (m, 1H), 3.33-3.52 (m, 3H), 3.81 (d, J=2.4 Hz, 1H), 4.34-4.72 (m, 2H), 5.44 (t, J=24 Hz, 1H), 6.70 (dd, J=2.4, 9.6 Hz, 1H), 6.78 (d, J=11.2 Hz, 1H), 6.93 (d, J=8.8 Hz, 1H), 7.38 (dd, J=2.4, 9.6 Hz, 1H), 7.43 (d, J=11.2 Hz, 1H), 7.55-7.75 (m, 4H), 8.02-8.10 (m, 1H), 8.49-8.64 (m, 1H). Chiral separation condition: MeOH contained 0.5% NH₄OH; (R,R)-Whelk-O1 (4.6×250 mm, 5 μm); retention time: 3.69 minute.

Compound 163-2: LC-MS (m/z): 647 [M+1]⁺; ¹H-NMR (CD₃OD, 400 MHz): δ (ppm) 0.79-0.88 (m, 2H), 1.09-1.50 (m, 6H) 1.59-1.63 (m, 5H), 1.82-1.96 (m, 2H), 2.58-2.75 (m, 1H), 3.01-3.18 (m, 2H), 3.28-3.45 (m, 2H), 4.13-4.46 (m, 1H), 5.30 (dd, J=10.0, 23.0 Hz, 1H), 6.63-6.70 (m, 2H), 7.32-7.62 (m, 7H), 7.92-7.97 (m, 1H), 8.45-8.54 (m, 1H). Chiral separation condition: MeOH contained 0.5% NH₄OH; (R,R)-Whelk-O1 (4.6×250 mm, 5 μm); retention time: 4.76 minute.

### Example 164

### Synthesis of (R)—N-(3-(4-aminopiperidin-1-yl)-1-(4′-chloro-[1,1′-biphenyl]-4-yl)-1,1-difluoro-3-oxopropan-2-yl)-6-(cyclopentyloxy)benzo[b]thiophene-2-sulfonamide (Compound 164-1), and (S)—N-(3-(4-aminopiperidin-1-yl)-1-(4′-chloro-[1,1′-biphenyl]-4-yl)-1,1-difluoro-3-oxopropan-2-yl)-6-(cyclopentyloxy)benzo[b]thiophene-2-sulfonamide (Compound 164-2)

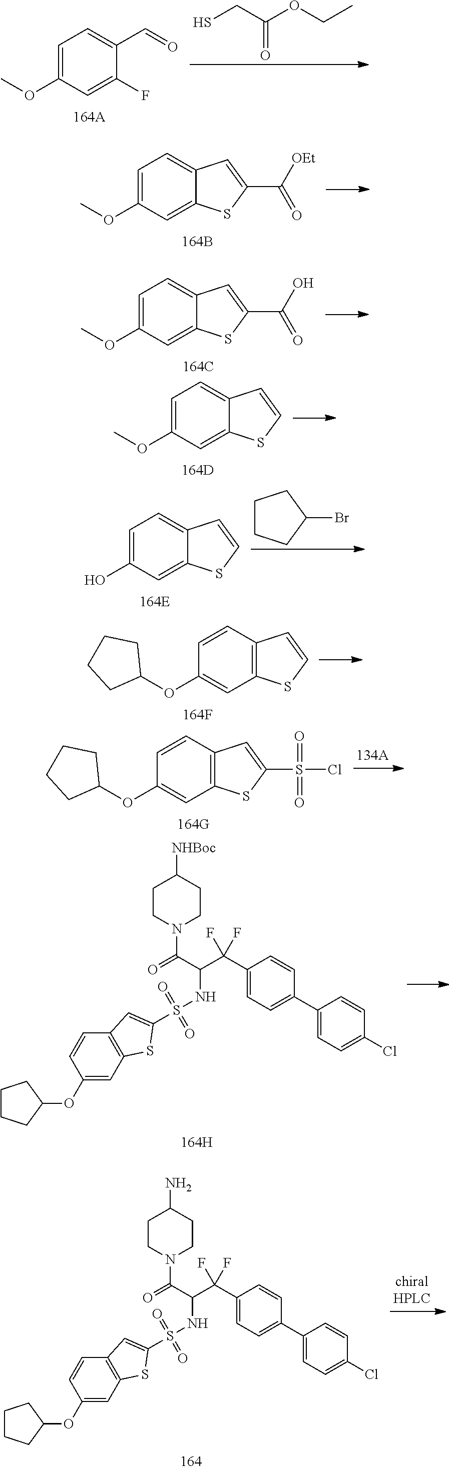

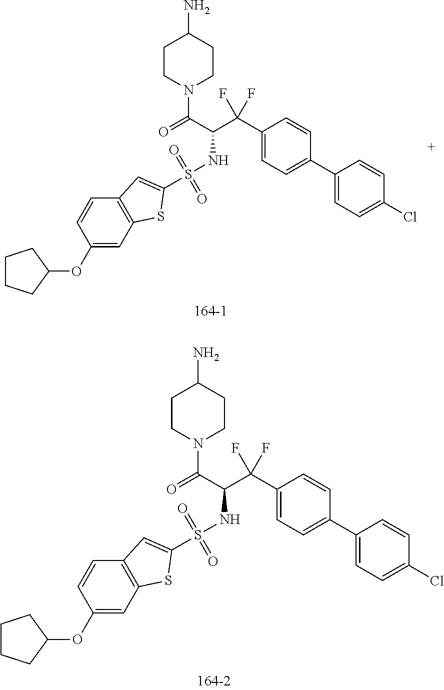 [SMILES: NC1CCN(C(=O)[C@@H](NS(=O)(=O)c2cc3ccc(OC4CCCC4)cc3s2)C(F)(F)c2ccc(-c3ccc(Cl)cc3)cc2)CC1 ; NC1CCN(C(=O)[C@H](NS(=O)(=O)c2cc3ccc(OC4CCCC4)cc3s2)C(F)(F)c2ccc(-c3ccc(Cl)cc3)cc2)CC1]

To a suspension of 2-fluoro-4-methoxybenzaldehyde, 164A, (5.8 g, 37.7 mmol) and potassium carbonate (6.24 g, 45.2 mmol) in dry DMF (50 mL) under nitrogen at 0° C. was slowly added ethyl thioglycolate (4.5 mL, 37.7 mmol). The reaction mixture was slowly warmed to room temperature, stirred at 25° C. for 16 hours, and heated at 60° C. for 5.5 hours. The reaction mixture was cooled down to room temperature, diluted with water (100 mL), stirred at room temperature for 30 minutes, and filtered. The solid was washed with water (100 mL×2) and dried under vacuum to afford Compound 164B. LC-MS (ESI) m/z: 237 [M+H]⁺; ¹H-NMR (CDCl₃, 400 MHz): δ (ppm) 1.40 (t, J=7.2 Hz, 3H), 3.87 (s, 3H), 4.38 (q, J=7.2 Hz, 2H), 7.01 (dd, J=8.4, 1.6 Hz, 1H), 7.26 (s, 1H), 7.72 (d, J=8.8 Hz, 1H), 7.95 (s, 1H).

To a solution of Compound 164B (3 g, 12.7 mmol) in hot ethanol (20 mL) was added a solution of potassium hydroxide (4.1 g, 76.3 mmol) in water (20 mL). The suspension was heated at reflux for 1 hour during which time the most of solid was dissolved. After cooled, to it was dropped an aqueous HCl solution (6 N, 10 mL). The ethanol was evaporated under reduced pressure and the residual solid was filtered. The filter cake was washed with water (20 mL) and dried to give Compound 164C. LC-MS (ESI) m/z: 209 [M+H]⁺.

A suspension of Compound 164C (1 g, 4.8 mmol) and copper powder (154 mg, 2.4 mmol) in quinoline (20 mL) was heated at 185° C. for 2 hours. After the reaction mixture was cooled down to room temperature, to it was added ethyl acetate (50 mL) and filtered. The filter cake was washed with ethyl acetate (50 mL×2). The combined organic layers was washed with an aqueous HCl solution (2 N, 20 mL×2), dried over anhydrous sodium sulfate, filtered, and evaporated under reduced pressure. The residue was purified by silica gel column chromatography to afford Compound 164D. ¹H-NMR (CDCl₃, 400 MHz): δ (ppm) 3.05 (s, 3H), 6.97-7.00 (m, 1H), 7.22-7.24 (m, 2H), 7.33 (m, 1H), 7.67 (d, J=8.8 Hz, 1H).

A mixture of Compound 164D (300 mg, 1.8 mmol) and pyridine hydrochloride (1.5 g) were stirred at 210° C. for 1 hour. After cooled down to room temperature, the reaction mixture was diluted with dichloromethane (50 mL), washed with water (50 mL) and brine (50 mL), dried over anhydrous sodium sulfate, filtered, and concentrated to yield a crude product, which was purified by silica gel column chromatography to furnish Compound 164E. LC-MS: (ESI) m/z: 151 [M+H]⁺; ¹H-NMR (DMSO-d₆, 400 MHz): δ (ppm) 6.19-6.20 (m, 1H), 6.90-6.93 (m, 1H), 7.19-7.20 (m, 1H), 7.30-7.31 (m, 1H), 7.62 (d, J=8.8 Hz, 1H).

Compound 164F was synthesized by employing the procedure described for Compound A4-1 using bromocyclopentane and Compound 164E in lieu of (bromomethyl)cyclopentane and Compound 2A. ¹H-NMR (CDCl₃, 400 MHz): δ (ppm) 1.58-1.62 (m, 2H), 1.77-1.88 (m, 6H), 4.75-4.77 (m, 1H), 6.92-6.95 (m, 1H), 7.18-7.19 (m, 2H), 7.28-7.29 (m, 1H), 7.62-7.64 (m, 1H).

To a solution of Compound 164F (500 mg, 2.29 mmol) in dry THF (20 mL) was added dropwise n-BuLi (2.4 M solution in hexane, 1.3 mL, 3.44 mmol) at −30° C. under nitrogen and stirred for 30 minutes. To the reaction mixture was added sulfuryl dichloride (618 mg, 4.58 mmol) and stirred at −30° C. for one hour. The reaction mixture was quenched with saturated NH₄C₁ solution (50 mL) and extracted with ethyl acetate (50 mL×3). The combined organic extracts were washed with water (50 mL) and brine (50 mL), dried over anhydrous sodium sulfate, filtered, and concentrated to yield a crude product, which was purified by silica gel column chromatography to furnish Compound 164G. ¹H-NMR (CDCl₃, 400 MHz): δ (ppm) 1.65-1.68 (m, 2H), 1.88-1.92 (m, 6H), 4.84-4.87 (m, 1H), 7.08-7.09 (m, 1H), 7.25-7.26 (m, 1H), 7.80 (d, J=9.2 Hz, 1H), 8.03 (s, 1H).

Compounds 164H and 164 were synthesized by employing the procedures described for Compounds 2D and 1 using Compounds 134A, 164G, and 164H in lieu of Compound B2, Compounds 2C, and 1A.

Compound 164H: LC-MS (ESI) m/z: 718 [M−55]⁺.

Compound 164 was separated with chiral HPLC to give Compound 164-1 and Compound 164-2.

Compound 164-1: LC-MS (ESI) m/z: 674 [M+H]⁺; ¹H-NMR (CD₃OD, 400 MHz): δ (ppm) 1.34-2.17 (m, 12H), 2.42-2.83 (m, 1H), 3.19-3.28 (m, 1H), 3.42-3.48 (m, 1H), 4.25-4.66 (m, 3H), 5.03-5.11 (m, 1H), 6.85-6.92 (m, 1H), 7.05-7.17 (m, 1H), 7.36-7.70 (m, 10H). Chiral separation condition: Methanol contained 1% Methanol Ammonia; OJ-H (4.6×250 mm, 5 μm); retention time: 5.25 minute.

Compound 164-2: LC-MS (ESI) m/z: 674 [M+H]⁺; ¹H-NMR (CD₃OD, 400 MHz): δ (ppm) 1.34-2.17 (m, 12H), 2.42-2.83 (m, 1H), 3.19-3.28 (m, 1H), 3.42-3.48 (m, 1H), 4.25-4.66 (m, 3H), 5.03-5.11 (m, 1H), 6.85-6.92 (m, 1H), 7.05-7.17 (m, 1H), 7.36-7.70 (m, 10H). Chiral separation condition: Methanol contained 1% Methanol Ammonia; OJ-H (4.6×250 mm, 5 μm); retention time: 4.36 minute.

### Example 165

### Synthesis of (S)—N-(3-(4-aminopiperidin-1-yl)-1-(4′-chloro-[1,1′-biphenyl]-4-yl)-1,1-difluoro-3-oxopropan-2-yl)-5-((cyclohexyloxy)methyl)thiophene-2-sulfonamide trifluoroacetate (Compound 165-1)), and (R)—N-(3-(4-aminopiperidin-1-yl)-1-(4′-chloro-[1,1′-biphenyl]-4-yl)-1,1-difluoro-3-oxopropan-2-yl)-5-((cyclohexyloxy) methyl)thiophene-2-sulfonamide trifluoroacetate (Compound 165-2)

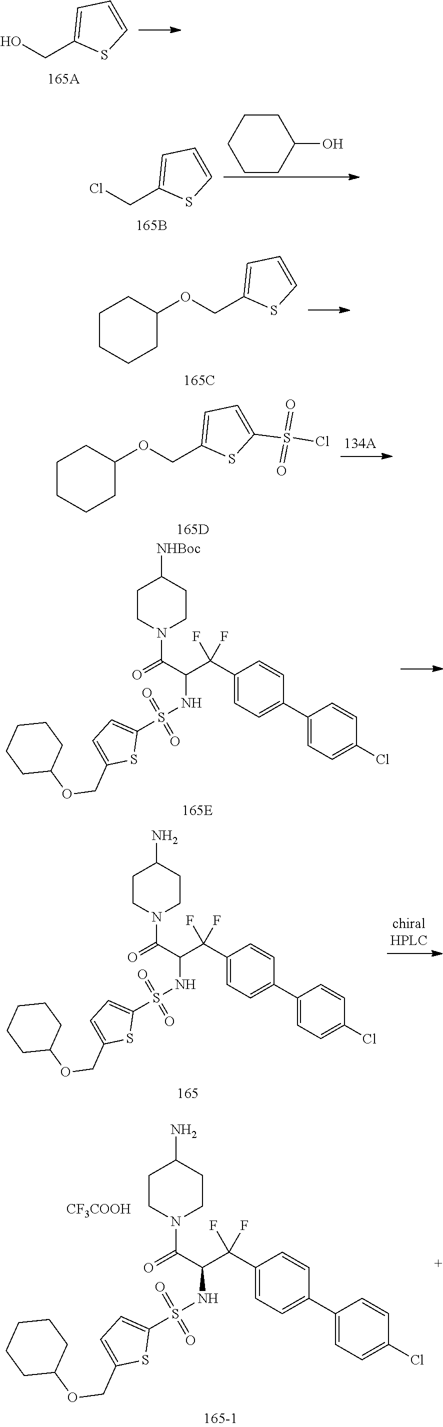

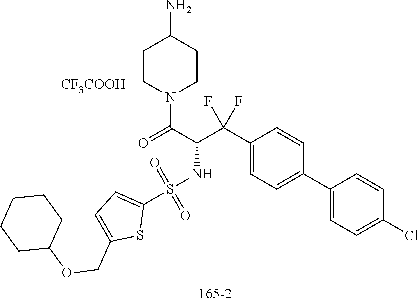 [SMILES: NC1CCN(C(=O)[C@@H](NS(=O)(=O)c2ccc(COC3CCCCC3)s2)C(F)(F)c2ccc(-c3ccc(Cl)cc3)cc2)CC1 ; "CF3COOH"]

To a mixture of thiophen-2-ylmethanol 165A (5 g, 44 mmol) in dichloromethane (50 mL) was added thionyl chloride (9.6 mL, 132 mmol) and DMF (0.5 mL, catalytical) at 0° C. The reaction mixture was stirred at 20° C. for 3 hours and then heated at reflux for 1 hour. The reaction mixture was concentrated under reduced pressure. The residue was diluted with dichloromethane (50 mL) and washed with saturated aqueous NaHCO₃ solution (100 mL). The organic layer was dried over anhydrous sodium sulfate, filtered, and concentrated to give Compound 165B. LC-MS (ESI) m/z: non-ionizable compound under routine conditions used.

To a mixture of cyclohexanol (5 g, 50 mmol) in dry DMF (50 mL) was added NaH (60% suspend in mineral oil, 2.5 g, 63 mmol) in several small portions at 0° C. The reaction mixture was stirred at room temperature for 2 hours. To the mixture was added a solution of Compound 165B (5.5 g, 42 mmol) in dry DMF (10 mL). The reaction mixture was stirred at room temperature for 2 hours, diluted with water (100 mL), and extracted with ether (50 mL×2). The combined organic extracts were washed with brine (50 mL×3), dried over anhydrous sodium sulfate, filtered, and concentrated to give a crude product, which was purified by silica gel column chromatography to afford Compound 165C. ¹H-NMR (CDCl₃, 400 MHz): δ (ppm) 1.18-1.38 (m, 5H), 1.50-1.55 (m, 1H), 1.74-1.76 (m, 2H), 1.91-1.94 (m, 2H), 3.35-3.42 (m, 1H), 4.71 (s, 2H), 6.95-6.97 (m, 2H), 7.25-7.26 (m, 1H).

Compounds 165D, 165E, and 165 were synthesized by employing the procedures described for Compounds 164G, 2D, and 1 using Compounds 165C, 134A, 165D, and 165E in lieu of Compound 164F, Compound B2, Compounds 2C, and 1A.

Compound 165D: LC-MS (ESI) m/z: 290 [M−Cl+MeNH₂]⁺ (sample was treated with methyl amine and then sent to LCMS). ¹H-NMR (CDCl₃, 400 MHz): δ (ppm) 1.23-1.41 (m, 6H), 1.75-1.79 (m, 2H), 1.91-1.94 (m, 2H), 3.42-3.47 (m, 1H), 4.71 (d, J=0.4 Hz, 2H), 6.97 (d, J=4.0 Hz, 1H), 7.75 (d, J=4.0 Hz, 1H).

Compound 165E: LC-MS (ESI) m/z: 652 [M−Boc+H]⁺.

Compound 165 was separated with chiral HPLC to give Compound 165-1 and Compound 165-2.

Compound 165-1: LC-MS (ESI) m/z: 652 [M+H]⁺; ¹H-NMR (CD₃OD, 400 MHz): δ (ppm) 1.27-1.65 (m, 8H), 1.73-2.17 (m, 6H), 2.58-2.79 (m, 1H), 3.09-3.23 (m, 1H), 3.34-3.39 (m, 2H), 4.21-4.27 (m, 1H), 4.42-4.61 (m, 3H), 5.00-5.09 (m, 1H), 6.82-6.89 (m, 1H), 7.26-7.33 (m, 1H), 7.49-7.53 (m, 3H), 7.59-7.71 (m, 5H). Chiral separation condition: MeOH contained 1% Methanol Ammonia; IC (250×4.6 mm, 5 μm); retention time: 5.24 minute.

Compound 165-2: LC-MS (ESI) m/z: 652 [M+H]⁺; ¹H-NMR (CD₃OD, 400 MHz): δ (ppm) 1.27-1.65 (m, 8H), 1.73-2.16 (m, 6H), 2.58-2.79 (m, 1H), 3.10-3.23 (m, 1H), 3.34-3.39 (m, 2H), 4.21-4.27 (m, 1H), 4.43-4.61 (m, 3H), 5.00-5.09 (m, 1H), 6.82-6.89 (m, 1H), 7.26-7.33 (m, 1H), 7.49-7.53 (m, 3H), 7.59-7.71 (m, 5H). Chiral separation condition: MeOH contained 1% Methanol Ammonia; IC (250×4.6 mm, 5 μm); retention time: 6.19 minute.

### Example 166

### Synthesis of (R)—N-(3-(4-aminopiperidin-1-yl)-1,1-difluoro-3-oxo-1-(4-(3,3,3-trifluoroprop-1-en-2-yl)phenyl)propan-2-yl)-6-(cyclopentyloxy)naphthalene-2-sulfonamide (Compound 166-1), and (S)—N-(3-(4-aminopiperidin-1-yl)-1,1-difluoro-3-oxo-1-(4-(3,3,3-trifluoroprop-1-en-2-yl)phenyl)propan-2-yl)-6-(cyclopentyloxy) naphthalene-2-sulfonamide (Compound 166-2)

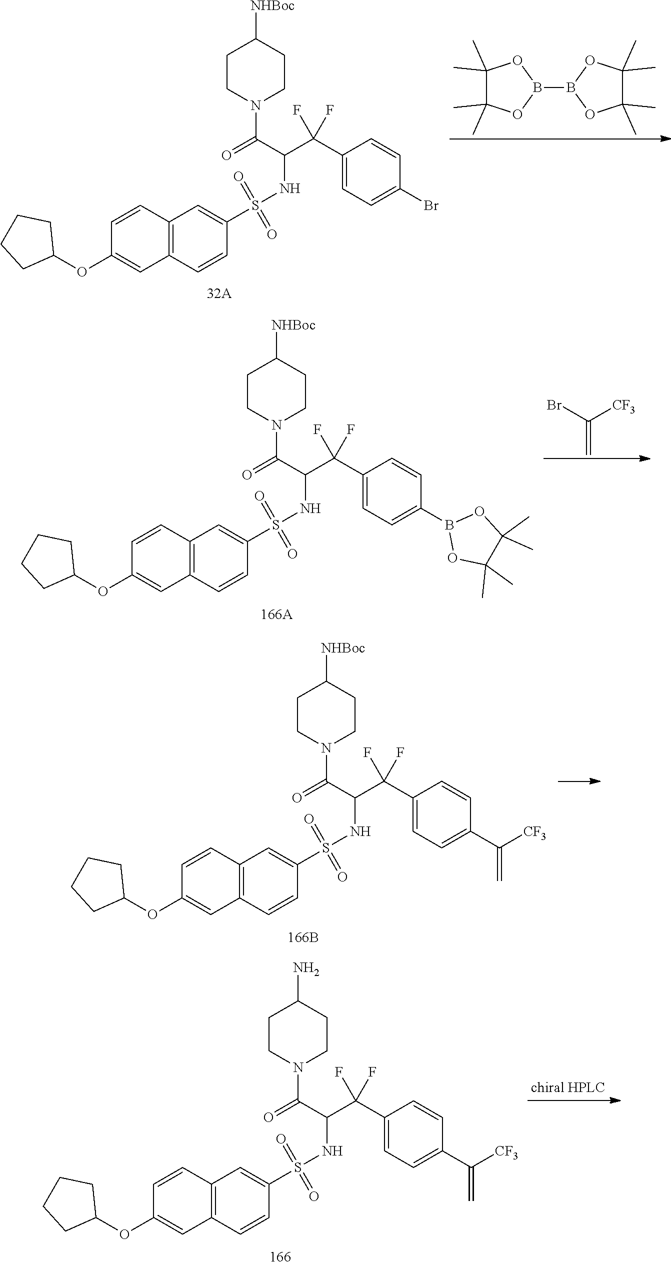

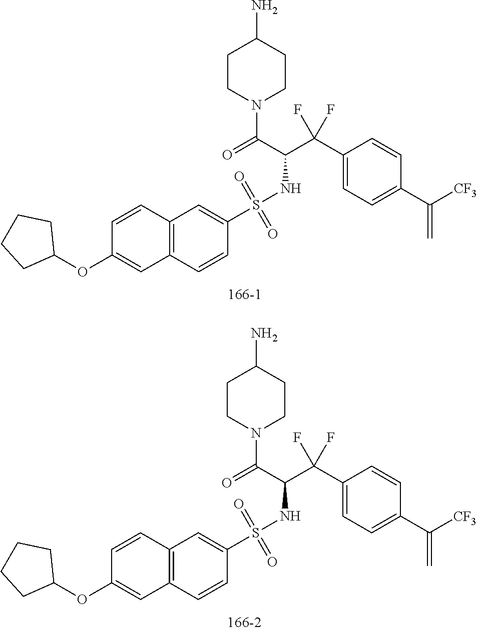 [SMILES: C=C(c1ccc(C(F)(F)[C@H](NS(=O)(=O)c2ccc3cc(OC4CCCC4)ccc3c2)C(=O)N2CCC(N)CC2)cc1)C(F)(F)F ; C=C(c1ccc(C(F)(F)[C@@H](NS(=O)(=O)c2ccc3cc(OC4CCCC4)ccc3c2)C(=O)N2CCC(N)CC2)cc1)C(F)(F)F]

A mixture of Compound 32A (270 mg, 0.367 mmol), 4,4,4′,4′,5,5,5′,5′-octamethyl-2,2′-bi(1,3,2-dioxaborolane) (186 mg, 0.734 mmol), Pd(dppf)Cl₂ (54 mg, 0.0734 mmol), and KOAc (144 mg, 1.467 mmol) in 1,4-dioxane (20 mL) was stirred at 80° C. overnight. The reaction mixture was cooled down to room temperature, diluted with water (100 mL), and extracted with ethyl acetate (50 mL×2). The combined organic extracts were washed with water (50 mL×3) and brine (50 mL), dried over anhydrous sodium sulfate, filtered, and concentrated under reduced pressure. The residue was purified by silica gel column chromatography to afford Compound 166A. LC-MS (ESI) m/z: 806 [M+Na]⁺.

A mixture of Compound 166A (200 mg, 0.288 mmol), 2-bromo-3,3,3-trifluoroprop-1-ene (252 mg, 1.44 mmol), Pd(PPh₃)₂Cl₂ (40 mg, 0.0576 mmol) and Cs₂CO₃ (188 mg, 0.576 mmol) in THF (10 mL) was stirred at 80° C. overnight. The reaction mixture was cooled down to room temperature, diluted with water (100 mL), and extracted with ethyl acetate (50 mL×2). The combined organic extracts were washed with water (50 mL×3) and brine (50 mL), dried over anhydrous sodium sulfate, filtered, and concentrated under reduced pressure. The residue was purified by silica gel column chromatography to afford Compound 166B. LC-MS (ESI) m/z: 696 [M−55]⁺.

Compound 166 was synthesized by employing the procedure described for Compound 6 using Compound 166B in lieu of Compound 6C. Compound 166 was separated by chiral HPLC to furnish Compound 166-1 and Compound 166-2.

Compound 166-1: LC-MS (ESI) m/z: 652 [M+H]⁺; ¹H-NMR (CD₃OD, 400 MHz): δ (ppm) 1.08-1.17 (m, 1H), 1.46-2.01 (m, 11H), 2.38-2.96 (m, 3H), 3.87-4.16 (m, 2H), 4.80-4.89 (m, 2H), 5.79-5.84 (m, 1H), 5.92-5.95 (m, 1H), 7.10-7.17 (m, 2H), 7.30-7.51 (m, 5H), 7.67-7.79 (m, 2H), 7.81 (s, 1H). Chiral separation condition: MeOH contained 0.5% NH₄OH; (R,R)-Whelk (4.6×250 mm, 5 μm); retention time: 3.65 minute.

Compound 166-2: LC-MS (ESI) m/z: 652 [M+H]⁺; ¹H-NMR (CD₃OD, 400 MHz): δ (ppm) 1.02-1.11 (m, 1H), 1.52-2.01 (m, 11H), 2.39-2.96 (m, 3H), 3.87-4.19 (m, 2H), 4.86-4.90 (m, 2H), 5.79-5.84 (m, 1H), 5.92-5.95 (m, 1H), 7.10-7.17 (m, 2H), 7.30-7.51 (m, 5H), 7.67-7.79 (m, 2H), 7.80 (s, 1H). Chiral separation condition: MeOH contained 0.5% NH₄OH; (R,R)-Whelk (4.6×250 mm, 5 μm); retention time: 4.48 minute.

### Example 167

### Synthesis of N-((2R)-3-(3-amino-8-azabicyclo[3.2.1]octan-8-yl)-1-(4-bromophenyl)-1,1-difluoro-3-oxopropan-2-yl)-6-(cyclopentyloxy)benzo[b]thiophene-2-sulfonamide trifluoroacetate (Compound 167-1), and N-((2 S)-3-(3-amino-8-azabicyclo[3.2.1]octan-8-yl)-1-(4-bromophenyl)-1,1-difluoro-3-oxopropan-2-yl)-6-(cyclopentyloxy)benzo[b]thiophene-2-sulfonamide trifluoroacetate (Compound 167-2)

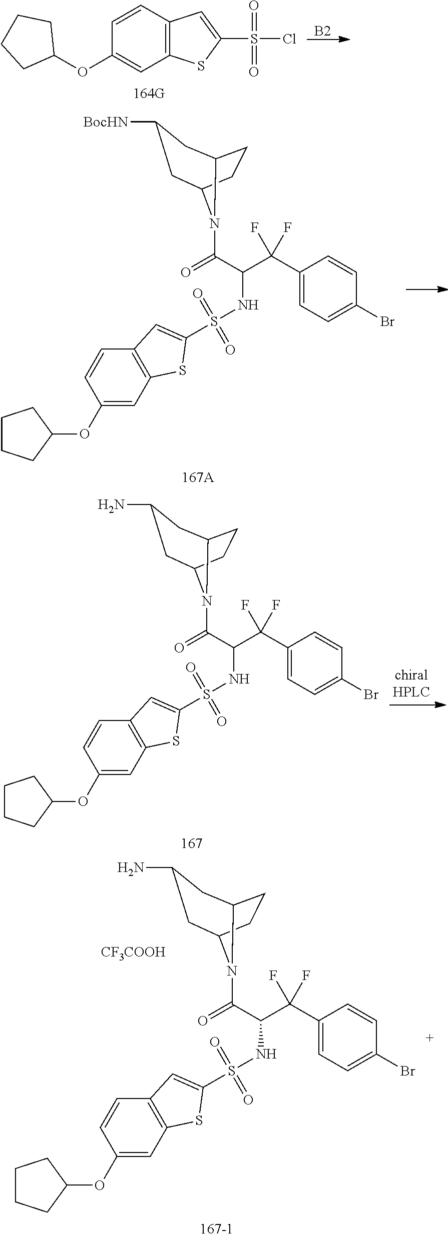

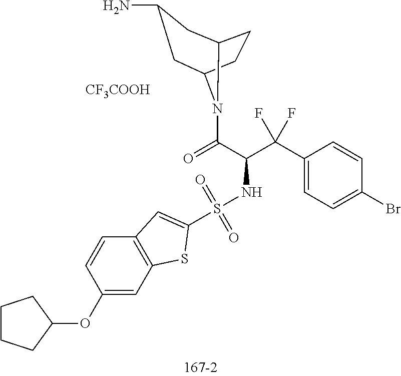 [SMILES: NC1CC2CCC(C1)N2C(=O)[C@H](NS(=O)(=O)c1cc2ccc(OC3CCCC3)cc2s1)C(F)(F)c1ccc(Br)cc1 ; "CF3COOH"]

Compounds 167A and 167 were synthesized by employing the procedures described for Compounds 2D and 1 using Compounds 164G and 167A in lieu of Compounds 2C and 1A.

Compound 167A: LC-MS (ESI) m/z: 712 [M−55]⁺.

Compound 167 was separated with chiral HPLC to give Compound 167-1 and Compound 167-2.

Compound 167-1: LC-MS (ESI) m/z: 668 [M+H]⁺; ¹H-NMR (CD₃OD, 400 MHz): δ (ppm) 1.49-2.16 (m, 16H), 3.59-3.71 (m, 1H), 4.50-4.80 (m, 3H), 4.90-4.92 (m, 1H), 7.03-7.07 (m, 1H), 7.29 (s, 2H), 7.37-7.45 (m, 2H), 7.54-7.66 (m, 2H), 7.73-7.77 (m, 1H). Chiral separation condition: Methanol contained 1% Methanol Ammonia; OJ-H (4.6×250 mm, 5 μm); retention time: 3.22 minute.

Compound 167-2: LC-MS (ESI) m/z: 668 [M+H]⁺; ¹H-NMR (CD₃OD, 400 MHz): δ (ppm) 1.49-2.16 (m, 16H), 3.59-3.71 (m, 1H), 4.50-4.80 (m, 3H), 4.90-4.92 (m, 1H), 7.03-7.07 (m, 1H), 7.29 (s, 2H), 7.37-7.45 (m, 2H), 7.54-7.66 (m, 2H), 7.73-7.77 (m, 1H). Chiral separation condition: Methanol contained 1% Methanol Ammonia; OJ-H (4.6×250 mm, 5 μm); column: OJ-H (4.6×250 mm, 5 μm), retention time: 2.17 minute.

### Example 168

### Synthesis of (R)—N-(3-(4-aminopiperidin-1-yl)-1-(4-chloro-3-cyclopropoxyphenyl)-1,1-difluoro-3-oxopropan-2-yl)-6-(cyclopentyloxy)naphthalene-2-sulfonamide trifluoroacetate (Compound 168-1), and (S)—N-(3-(4-aminopiperidin-1-yl)-1-(4-chloro-3-cyclopropoxyphenyl)-1,1-difluoro-3-oxopropan-2-yl)-6-(cyclopentyloxy) naphthalene-2-sulfonamide trifluoroacetate (Compound 168-2)

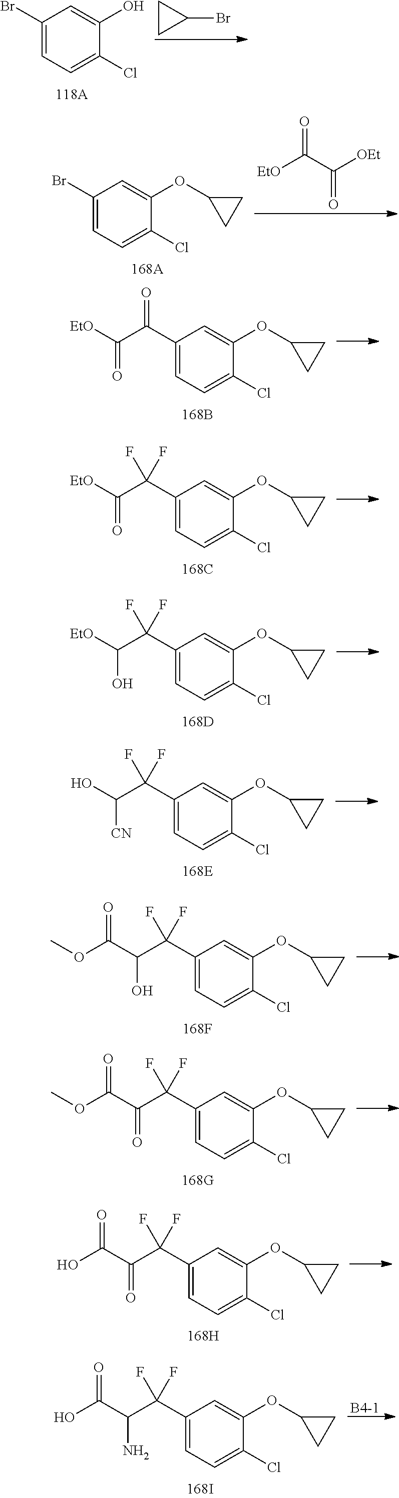

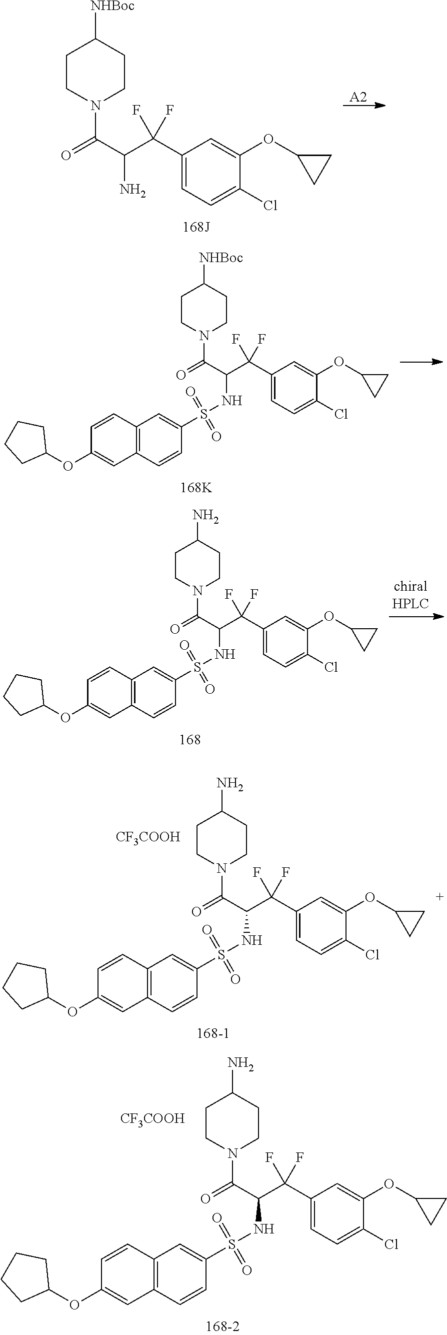

Compounds 168A, 168B, 168C, 168D, 168E, 168F, 168G, 168H, 168I, 168J, 168K, and 168 were synthesized by employing the procedures described for Compound A4-1, Compound 9B, Compound B2-3, Compounds 128G, 128H, 45G, Compound B6-3, Compounds 451, 80A, Compound B4, Compounds 2D, and 1 using bromocyclopropane, Compounds 118A, 168A, 168B, 168C, 168D, 168E using MeOH as solvent, 168F, 168G, 168H, 168I, 168J, Compound A2, and 168K in lieu of (bromomethyl)cyclopentane, Compounds 2A, 9A, Compound B2-2, Compounds 128F, 128G, 45F using EtOH as solvent, Compound B6-2, Compounds 45H, 51J, Compounds B2-6, B2, Compounds 2C, and 1A.

Compound 168A: ¹H-NMR (CDCl₃, 400 MHz): δ (ppm) 1.37 (t, J=4.4 Hz, 4H), 3.76-3.80 (m, 1H), 7.04 (dd, J=8.4, 2.0 Hz, 1H), 7.19 (d, J=8.4 Hz, 1H), 7.41 (d, J=2.4 Hz, 1H).

Compound 168B: LC-MS ESI (m/z): 269 [M+H]⁺; ¹H-NMR (CDCl₃, 400 MHz): δ (ppm) 0.84-0.88 (m, 4H), 1.32 (t, J=6.8 Hz, 3H), 3.82-3.85 (m, 1H), 4.28-4.34 (m, 2H), 7.15 (d, J=8.4 Hz, 1H), 7.41 (d, J=8.0 Hz, 1H), 7.51 (s, 1H).

Compound 168C: ¹H-NMR (CDCl₃, 400 MHz): δ (ppm) 0.77-0.85 (m, 4H), 1.32 (t, J=6.8 Hz, 3H), 3.82-3.86 (m, 1H), 4.01-4.12 (m, 2H), 7.15 (d, J=8.4 Hz, 1H), 7.42 (d, J=8.0 Hz, 1H), 7.51 (d, J=0.8 Hz, 1H).

Compound 168D was used directly in next step without further purification (yield 100%). LC-MS (ESI) m/z: non-ionizable compound under routine conditions used.

Compound 168E: LC-MS ESI (m/z): 274 [M+H]⁺; ¹H-NMR (CDCl₃, 400 MHz): δ (ppm) 0.80-0.88 (m, 4H), 3.83-3.87 (m, 1H), 4.83 (t, J=8.4 Hz, 1H), 7.11 (d, J=8.0 Hz, 1H), 7.45-7.50 (m, 2H).

Compound 168F: LC-MS ESI (m/z): 329 [M+Na]⁺; ¹H-NMR (CDCl₃, 400 MHz): δ (ppm) 0.82-0.87 (m, 4H), 4.23 (d, J=8.0 Hz, 1H), 3.77-3.82 (m, 1H), 3.85 (s, 3H), 4.51-4.58 (m, 1H), 7.02 (d, J=8.0 Hz, 1H), 7.39-7.41 (m, 2H).

Compound 168G: LC-MS ESI (m/z): 345 [M+H₂O+Na]⁺; ¹H-NMR (CDCl₃, 400 MHz): δ (ppm) 0.84-0.88 (m, 4H), 3.80-3.85 (m, 1H), 3.99 (s, 3H), 7.11-7.16 (m, 1H), 7.40 (d, J=8.8 Hz, 1H), 7.51 (d, J=2.0 Hz, 1H).

Compound 168H: LC-MS ESI (m/z): 289 [M−H]⁻.

Compound 1681 hydrochloride: LC-MS ESI (m/z): 292 [M+H]⁺.

Compound 168J: LC-MS ESI (m/z): 474 [M+H]⁺; ¹H-NMR (CDCl₃, 400 MHz): δ (ppm) 0.84-0.87 (m, 4H), 1.26 (s, 9H), 1.44-1.68 (m, 2H), 1.91-1.98 (m, 2H), 2.68-2.81 (m, 1H), 3.08-3.21 (m, 1H), 3.48-3.79 (m, 4H), 4.33-4.54 (m, 4H), 6.97 (d, J=8.8 Hz, 1H), 7.33-7.41 (m, 2H).

Compound 168K: LC-MS ESI (m/z): 692 [M−55]⁺.

Compound 168 was separated with chiral HPLC to give Compound 168-1 and Compound 168-2.

Compound 168-1: LC-MS (ESI) m/z: 648 [M+H]⁺; ¹H-NMR (CD₃OD, 400 MHz): δ (ppm) 0.58-0.82 (m, 4H), 1.32-2.16 (m, 12H), 2.78-3.05 (m, 1H), 3.17-3.24 (m, 1H), 3.33-3.76 (m, 2H), 4.14-4.66 (m, 2H), 4.95-5.08 (m, 2H), 6.92-7.43 (m, 6H), 7.69-7.89 (m, 2H), 8.11-8.18 (m, 1H). Chiral separation condition: MeOH contained 0.5% NH₄OH; (R, R)-Whelk-O1 (4.6×150 mm, 5 μm); retention time: 5.03 minute.

Compound 168-2: LC-MS (ESI) m/z: 648 [M+H]⁺; ¹H-NMR (CD₃OD, 400 MHz): δ (ppm) 0.57-0.82 (m, 4H), 1.281.51 (m, 2H), 1.71-2.18 (m, 10H), 2.76-3.06 (m, 1H), 3.17-3.23 (m, 1H), 3.31-3.76 (m, 2H), 4.14-4.62 (m, 2H), 4.93-5.08 (m, 2H), 6.91-7.51 (m, 6H), 7.67-7.88 (m, 2H), 8.09-8.16 (m, 1H). Chiral separation condition: MeOH contained 0.5% NH₄OH; (R, R)-Whelk-O1 (4.6×150 mm, 5 μm); retention time: 5.71 minute.

### Example 169

### Synthesis of (R)—N-(3-(4-aminopiperidin-1-yl)-1-(4-chloro-3-propoxyphenyl)-1,1-difluoro-3-oxopropan-2-yl)-6-(cyclopentyloxy)naphthalene-2-sulfonamide trifluoroacetate (Compound 169-1), and (S)—N-(3-(4-aminopiperidin-1-yl)-1-(4-chloro-3-propoxyphenyl)-1,1-difluoro-3-oxopropan-2-yl)-6-(cyclopentyloxy) naphthalene-2-sulfonamide trifluoroacetate (Compound 169-2)

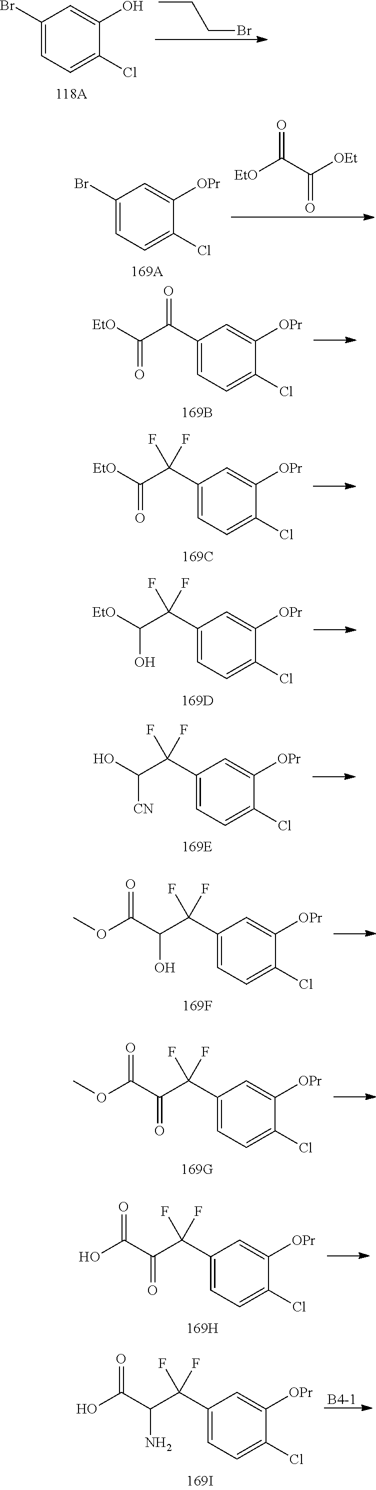

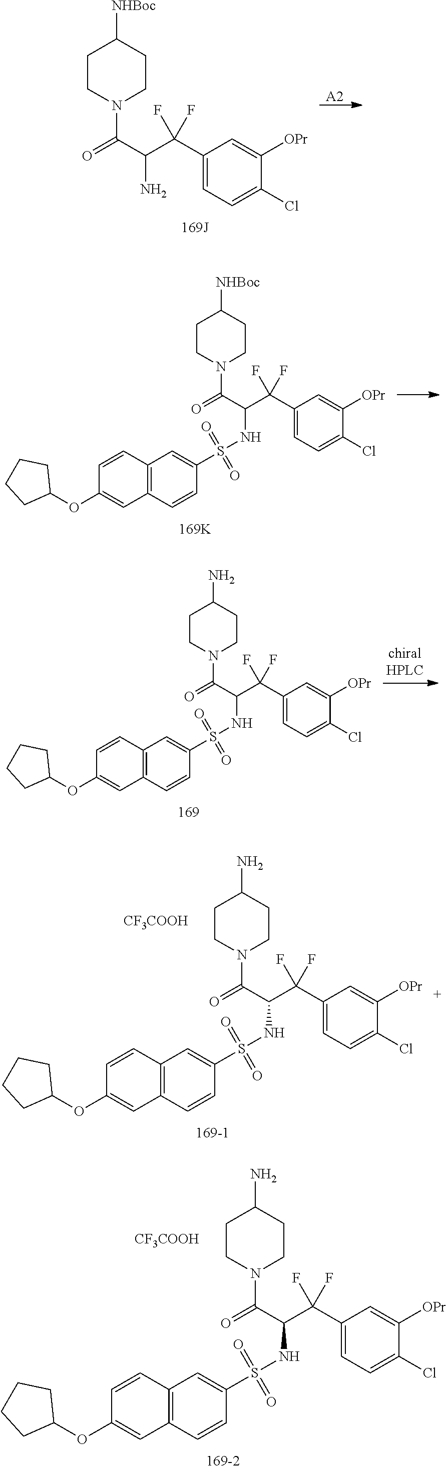

Compounds 169A, 169B, 169C, 169D, 169E, 169F, 169G, 169H, 169I, 169J, 169K, and 169 were synthesized by employing the procedures described for Compound A4-1, Compound 9B, Compound B2-3, Compounds 128G, 128H, 45G, Compound B6-3, Compounds 451, 80A, Compound B4, Compounds 2D, and 1 using 1-bromopropane Compounds 118A, 169A, 169B, 169C, 169D, 169E using MeOH as solvent, 169F, 169G, 169H, 169I, 169J, Compound A2, and 169K in lieu of (bromomethyl)cyclopentane, Compounds 2A, 9A, Compound B2-2, Compounds 128F, 128G, 45F using EtOH as solvent, Compound B6-2, Compounds 45H, 51J, Compounds B2-6, B2, Compounds 2C, and 1A.

Compound 169A: ¹H-NMR (CDCl₃, 400 MHz): δ (ppm) 1.37 (t, J=7.6 Hz, 3H), 1.83-1.89 (m, 2H), 3.97 (t, J=6.8 Hz, 2H), 6.99-7.03 (m, 2H), 7.20-7.26 (m, 1H).

Compound 169B: LC-MS ESI (m/z): 271 [M+H]⁺; ¹H-NMR (CDCl₃, 400 MHz): δ (ppm) 1.07 (t, J=7.2 Hz, 3H), 1.43 (t, J=7.2 Hz, 3H), 1.88-1.90 (m, 2H), 4.06 (t, J=6.8 Hz, 2H), 4.44-4.46 (m, 2H), 7.49-7.51 (m, 2H), 7.58 (s, 1H),

Compound 169C: ¹H-NMR (CDCl₃, 400 MHz): δ (ppm) 1.08 (t, J=7.2 Hz, 3H), 1.31 (t, J=7.2 Hz, 3H), 1.85-1.90 (m, 2H), 4.01 (t, J=6.8 Hz, 2H), 4.31 (q, J=7.2 Hz, 2H), 7.10-7.14 (m, 2H), 7.43 (d, J=8.8 Hz, 1H).

Compound 169D: LC-MS (ESI) m/z: non-ionizable compound under routine conditions used.

Compound 169E: LC-MS ESI (m/z): 276 [M+H]⁺; ¹H-NMR (CDCl₃, 400 MHz): δ (ppm) 1.08 (t, J=7.2 Hz, 3H), 1.85-1.90 (m, 2H), 3.35 (d, J=8.0 Hz, 1H), 4.03 (t, J=6.8 Hz, 2H), 4.80-4.82 (m, 1H), 7.10-7.14 (m, 2H), 7.43 (d, J=8.8 Hz, 1H).

Compound 169F: ¹H-NMR (CDCl₃, 400 MHz): δ (ppm) 1.08 (t, J=7.6 Hz, 3H), 1.29 (t, J=7.2 Hz, 3H), 1.85-1.90 (m, 2H), 3.23 (d, J=8.0 Hz, 1H), 4.00 (t, J=6.8 Hz, 2H), 4.30-4.32 (m, 2H), 4.48-4.52 (m, 1H), 7.10-7.14 (m, 2H), 7.42 (d, J=8.8 Hz, 1H).

Compound 169G hydrate: LC-MS ESI (m/z): 321 [M+H]⁺; ¹H-NMR (CDCl₃, 400 MHz): δ (ppm) 1.08 (t, J=7.6 Hz, 3H), 1.39 (t, J=7.2 Hz, 3H), 1.85-1.90 (m, 2H), 4.00-4.03 (m, 2H), 4.30-4.32 (m, 2H), 7.10-7.14 (m, 2H), 7.42 (d, J=8.8 Hz, 1H).

Compound 169H: LC-MS ESI (m/z): 291 [M−H]⁻.

Compound 1691 hydrochloride: LC-MS ESI (m/z): 294 [M+H]⁺.

Compound 169J: LC-MS ESI (m/z): 476 [M+H]⁺.

Compound 169K was used directly in the next step without further purification. LC-MS ESI (m/z): 694 [M−55]⁺.

Compound 169 was separated with chiral HPLC to give Compound 169-1 and Compound 169-2.

Compound 169-1: LC-MS (ESI) m/z: 650 [M+H]⁺; ¹H-NMR (CD₃OD, 400 MHz): δ (ppm) 0.86-1.05 (m, 3H), 1.30-2.09 (m, 14H), 2.75-3.09 (m, 1H), 3.23-3.26 (m, 1H), 3.32-3.81 (m, 3H), 4.21-4.66 (m, 2H), 4.88-5.08 (m, 2H), 6.78-7.84 (m, 8H), 8.05-8.13 (m, 1H). Chiral separation condition: MeOH contained 0.5% NH₄OH; IC (4.6×150 mm, 5 μm); retention time: 4.51 minute.

Compound 169-2: LC-MS (ESI) m/z: 650 [M+H]⁺; ¹H-NMR (CD₃OD, 400 MHz): δ (ppm) 0.88-1.07 (m, 3H), 1.31-2.19 (m, 14H), 2.78-3.13 (m, 1H), 3.23-3.26 (m, 1H), 3.32-3.81 (m, 3H), 4.21-4.66 (m, 2H), 4.88-5.08 (m, 2H), 6.78-7.84 (m, 8H), 8.08-8.15 (m, 1H). Chiral separation condition: MeOH contained 0.5% NH₄OH; IC (4.6×150 mm, 5 μm); retention time: 5.71 minute.

### Example 170

### Synthesis of (R)—N-(3-(4-aminopiperidin-1-yl)-1-(4-chloro-3-(cyclopentyloxy)phenyl)-1,1-difluoro-3-oxopropan-2-yl)-6-(cyclopentyloxy)naphthalene-2-sulfonamide trifluoroacetate (Compound 170-1), and (S)—N-(3-(4-aminopiperidin-1-yl)-1-(4-chloro-3-(cyclopentyloxy)phenyl)-1,1-difluoro-3-oxopropan-2-yl)-6-(cyclopentyloxy) naphthalene-2-sulfonamide trifluoroacetate (Compound 170-2)

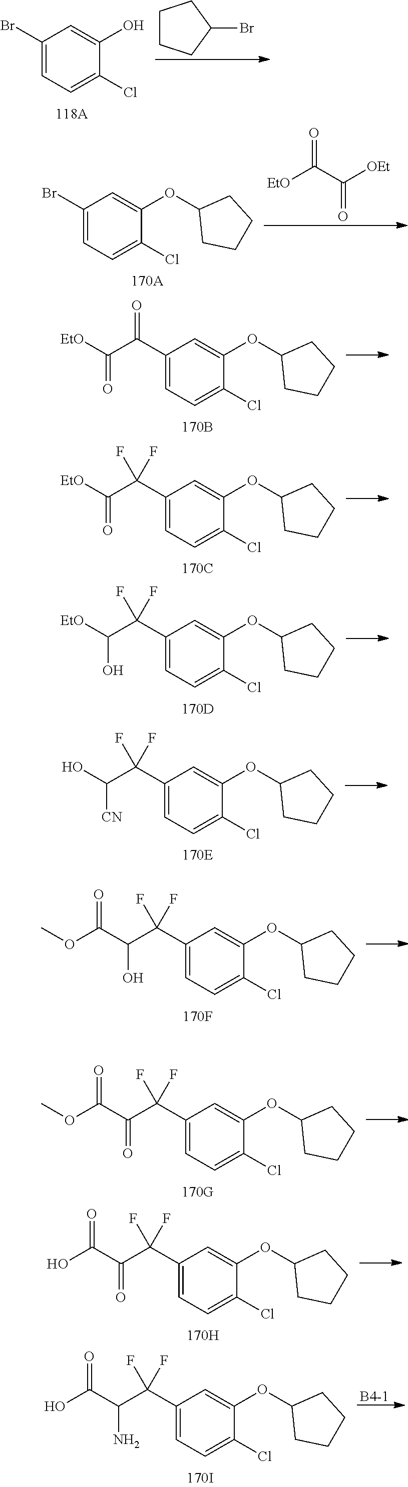

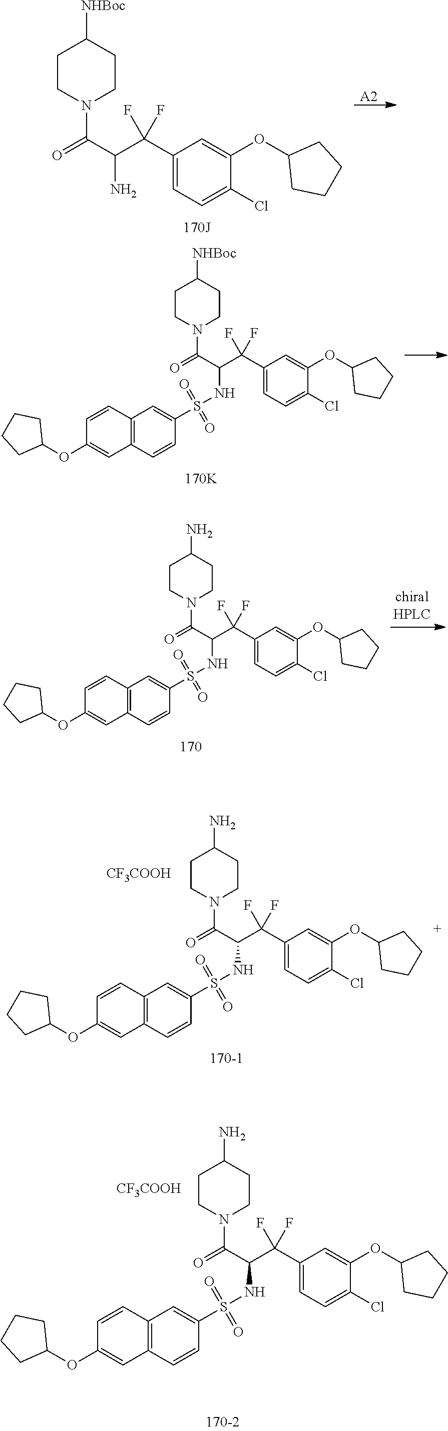

Compounds 170A, 170B, 170C, 170D, 170E, 170F, 170G, 170H, 170I, 170J, 170K, and 170 were synthesized by employing the procedures described for Compound A4-1, Compound 9B, Compound B2-3, Compounds 128G, 128H, 45G, Compound B6-3, Compounds 451, 80A, Compound B4, Compounds 2D, and 1 using bromocyclopentane, Compounds 118A, 170A, 170B, 170C, 170D, 170E using MeOH as solvent, 170F, 170G, 170H, 170I, 170J, Compound A2, and 170K in lieu of (bromomethyl)cyclopentane, Compounds 2A, 9A, Compound B2-2, Compounds 128F, 128G, 45F using EtOH as solvent, Compound B6-2, Compounds 45H, 51J, Compounds B2-6, B2, Compounds 2C, and 1A.

Compound 170A: ¹H-NMR (CDCl₃, 400 MHz): δ (ppm) 1.63-1.67 (m, 2H), 1.83-1.93 (m, 6H), 4.77-4.81 (m, 1H), 6.99 (dd, J=8.4, 2.4 Hz, 1H), 7.05 (d, J=2.0 Hz, 1H), 7.20 (d, J=8.4 Hz, 1H).

Compound 170B: LC-MS ESI (m/z): 319 [M+Na]⁺; ¹H-NMR (CDCl₃, 400 MHz): δ (ppm) 1.44 (t, J=7.2 Hz, 3H), 1.65-1.69 (m, 2H), 1.84-1.96 (m, 6H), 4.46 (q, J=7.2 Hz, 2H), 4.89-4.93 (m, 1H), 7.46-7.49 (m, 2H), 7.60 (s, 1H).

Compound 170C: ¹H-NMR (CDCl₃, 400 MHz): δ (ppm) 1.32 (t, J=7.2 Hz, 3H), 1.64-1.68 (m, 2H), 1.83-1.94 (m, 6H), 4.31 (q, J=7.2 Hz, 2H), 4.84-4.87 (m, 1H), 7.10 (d, J=8.4 Hz, 1H), 7.14 (s, 1H), 7.40 (d, J=8.0 Hz, 1H).

Compound 170D: LC-MS (ESI) m/z: non-ionizable compound under routine conditions used.

Compound 170E: LC-MS ESI (m/z): 302 [M+H]⁺; ¹H-NMR (CDCl₃, 400 MHz): δ (ppm) 1.62-1.68 (m, 2H), 1.84-1.95 (m, 6H), 4.80-4.84 (m, 1H), 4.85-4.90 (m, 1H), 7.00-7.12 (m, 2H), 7.41-7.48 (m, 1H).

Compound 170F: LC-MS ESI (m/z): 333 [M−H]; ¹H-NMR (CDCl₃, 400 MHz): δ (ppm) 1.65-1.67 (m, 2H), 1.85-1.95 (m, 6H), 3.22 (d, J=7.6 Hz, 1H), 3.85 (s, 3H), 4.83-4.87 (m, 1H), 6.96-7.12 (m, 2H), 7.40-7.48 (m, 1H).

Compound 170G hydrate: LC-MS ESI (m/z): 333 [M+H]⁺; ¹H-NMR (CDCl₃, 400 MHz): δ (ppm) 1.61-1.66 (m, 2H), 1.86-1.93 (m, 6H), 3.95-3.99 (m, 3H), 4.83-4.89 (m, 1H), 7.15-7.23 (m, 2H), 7.39-7.45 (m, 1H).

Compound 170H: LC-MS ESI (m/z): 317 [M−H]⁻.

Compound 1701 hydrochloride: LC-MS ESI (m/z): 318 [M−H]⁻.

Compound 170J: LC-MS ESI (m/z): 502 [M+H]⁺.

Compound 170K: LC-MS ESI (m/z): 720 [M−55]⁺.

Compound 170 was separated with chiral HPLC to give Compound 170-1 and Compound 170-2.

Compound 170-1: LC-MS (ESI) m/z: 676 [M+H]⁺; ¹H-NMR (CD₃OD, 400 MHz): δ (ppm) 1.49-2.16 (m, 20H), 2.77-3.07 (m, 1H), 3.25-3.44 (m, 2H), 4.17-4.74 (m, 3H), 4.96-5.02 (m, 2H), 6.82-7.01 (m, 2H), 7.13-7.30 (m, 3H), 7.37-7.51 (m, 1H), 7.66-7.76 (m, 1H), 7.79-7.87 (m, 1H), 8.06-8.15 (m, 1H). Chiral separation condition: MeOH contained 0.5% NH₄OH); OJ-H (4.6×150 mm, 5 μm); retention time: 4.70 minute.

Compound 170-2: LC-MS (ESI) m/z: 676 [M+H]⁺; ¹H-NMR (CD₃OD, 400 MHz): δ (ppm) 1.49-2.14 (m, 20H), 2.77-3.07 (m, 1H), 3.22-3.44 (m, 2H), 4.17-4.74 (m, 3H), 4.94-5.02 (m, 2H), 6.81-7.28 (m, 2H), 7.36-7.51 (m, 3H), 7.52-7.65 (m, 1H), 7.66-7.76 (m, 1H), 7.79-7.87 (m, 1H), 8.06-8.14 (m, 1H). Chiral separation condition: MeOH contained 0.5% NH₄OH); OJ-H (4.6×150 mm, 5 μm); retention time: 3.51 minute.

### Example 171

### Synthesis of N—((R)-3-((S)-3-aminopyrrolidin-1-yl)-1-(4′-chloro-[1,1′-biphenyl]-4-yl)-1,1-difluoro-3-oxopropan-2-yl)-4-(cyclohexylmethoxy)benzenesulfonamide trifluoroacetate (Compound 171-1), and N—((S)-3-((S)-3-aminopyrrolidin-1-yl)-1-(4′-chloro-[1,1′-biphenyl]-4-yl)-1,1-difluoro-3-oxopropan-2-yl)-4-(cyclohexylmethoxy) benzenesulfonamide trifluoroacetate (Compound 171-2)

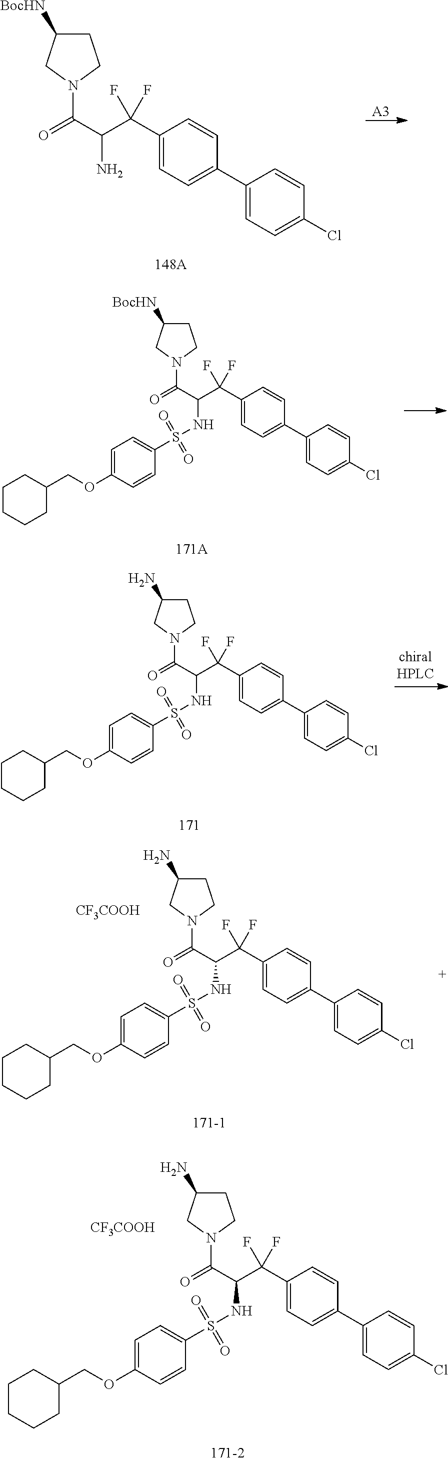

Compounds 171A and 171 were synthesized by employing the procedures described for Compounds 2D and 1 using Compound 148A, Compound A3, and Compound 171A in lieu of Compound B2, Compounds 2C, and 1A.

Compound 171A: LC-MS (ESI) m/z: 754 [M+Na]⁺.

Compound 171 was separated with chiral HPLC to give Compound 171-1 and Compound 171-2.

Compound 171-1: LC-MS (ESI) m/z: 632 [M+H]⁺; ¹H-NMR (DMSO-d₆, 400 MHz): δ (ppm) 0.88-0.94 (m, 2H), 1.15-1.23 (m, 3H), 1.63-1.67 (m, 6H), 1.867-2.18 (m, 2H), 3.05-3.22 (m, 1H), 3.51-3.87 (m, 6H), 4.53-4.79 (m, 1H), 6.78-6.87 (m, 2H), 7.41-7.48 (m, 4H), 7.55-7.61 (m, 3H), 7.68-7.77 (m, 3H), 8.20 (s, 3H), 8.64-8.67 (m, 1H). Chiral separation condition: MeOH contained 0.5% NH₄OH; IC (4.6×150 mm, 5 μm); retention time: 6.22 minute.

Compound 171-2: LC-MS (ESI) m/z: 632 [M+H]⁺; ¹H-NMR (DMSO-d₆, 400 MHz): δ (ppm) 0.915-0.94 (m, 2H), 1.19-1.43 (m, 3H), 1.69-1.71 (m, 6H), 1.77-2.25 (m, 2H), 3.14-3.42 (m, 2H), 3.55-3.78 (m, 5H), 4.60-4.63 (m, 1H), 6.82-6.85 (m, 2H), 7.41-7.48 (m, 4H), 7.55 (d, J=8.4 Hz, 2H), 7.63-7.66 (m, 2H), 7.72-7.75 (m, 2H), 8.11-8.20 (m, 3H), 8.61-8.77 (m, 1H). Chiral separation condition: MeOH contained 0.5% NH₄OH; IC (4.6×150 mm, 5 μm); retention time: 4.86 minute.

### Example 172

### Synthesis of (R)—N-(3-(4-aminopiperidin-1-yl)-1-(4-chloro-3-isopropoxyphenyl)-1,1-difluoro-3-oxopropan-2-yl)-6-(cyclopentyloxy)naphthalene-2-sulfonamide trifluoroacetate (Compound 172-1), and (S)—N-(3-(4-aminopiperidin-1-yl)-1-(4-chloro-3-isopropoxyphenyl)-1,1-difluoro-3-oxopropan-2-yl)-6-(cyclopentyloxy) naphthalene-2-sulfonamide trifluoroacetate (Compound 172-2)

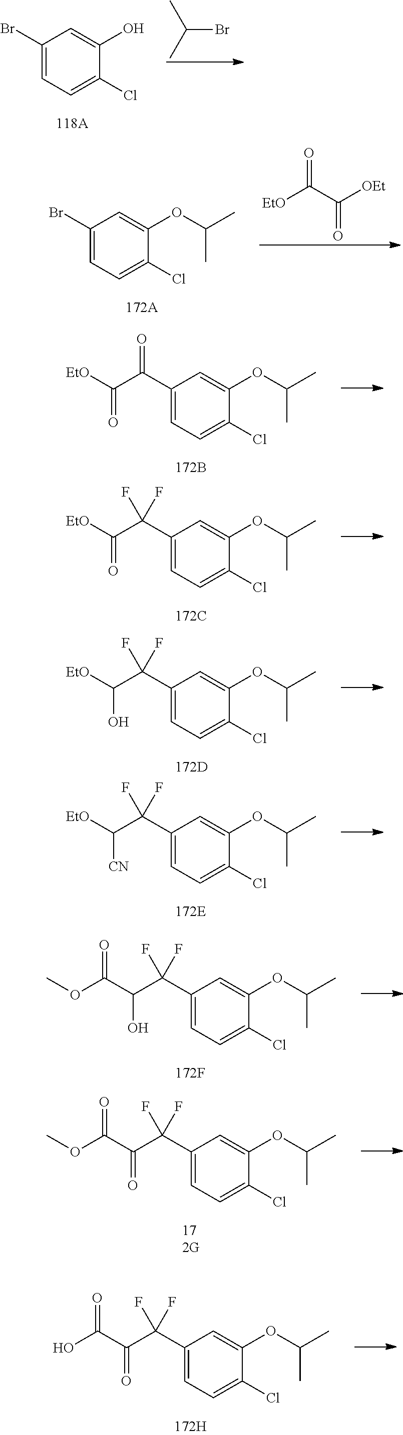

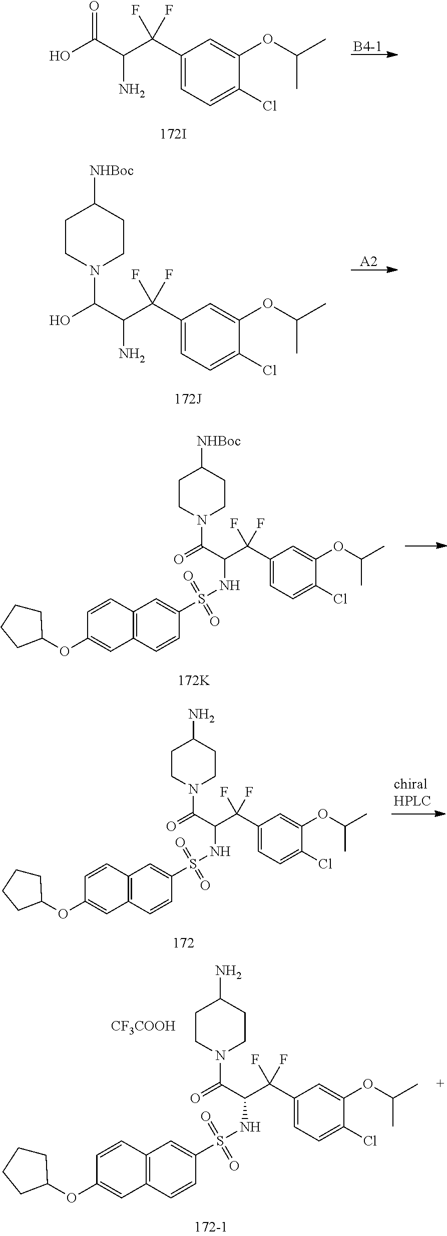

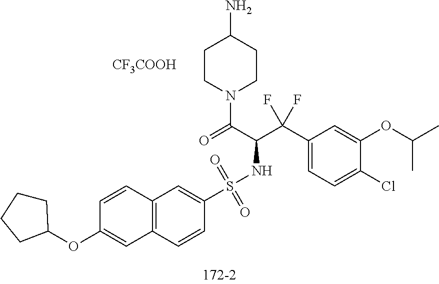 [SMILES: CC(C)Oc1cc(C(F)(F)[C@@H](NS(=O)(=O)c2ccc3cc(OC4CCCC4)ccc3c2)C(=O)N2CCC(N)CC2)ccc1Cl ; "CF3COOH"]

Compounds 172A, 172B, 172C, 172D, 172E, 172F, 172G, 172H, 172I, 172J, 172K, and 172 were synthesized by employing the procedures described for Compound A4-1, Compound 9B, Compound B2-3, Compounds 128G, 128H, 45G, Compound B6-3, Compounds 45I, 80A, Compound B4, Compounds 2D, and 1 using 2-bromopropane, Compounds 118A, 172A, 172B, 172C, 172D, 172E using MeOH as solvent, 172F, 172G, 172H, 172I, 172J, Compound A2, and 172K in lieu of (bromomethyl)cyclopentane, Compounds 2A, 9A, Compound B2-2, Compounds 128F, 128G, 45F using EtOH as solvent, Compound B6-2, Compounds 45H, 51J, Compounds B2-6, B2, Compounds 2C, and 1A.

Compound 172A: ¹H-NMR (CDCl₃, 400 MHz): δ (ppm) 1.37 (d, J=6.0 Hz, 6H), 4.48-4.57 (m, 1H), 6.99 (dd, J=8.4, 2.0 Hz, 1H), 7.06 (d, J=2.0 Hz, 1H), 7.20 (d, J=8.4 Hz, 1H).

Compound 172B: LC-MS ESI (m/z): 271 [M+H]⁺; ¹H-NMR (CDCl₃, 400 MHz): δ (ppm) 1.33 (d, J=6.4 Hz, 6H), 1.35 (t, J=7.2 Hz, 3H), 4.37 (q, J=7.2 Hz, 2H), 4.55-4.64 (m, 1H), 7.41-7.43 (m, 2H), 7.53 (d, J=1.6 Hz, 1H),

Compound 172C: LC-MS (ESI) m/z: non-ionizable compound under routine conditions used; ¹H-NMR (CDCl₃, 400 MHz): δ (ppm) 1.31 (t, J=7.2 Hz, 3H), 1.39 (d, J=6.0 Hz, 6H), 4.31 (q, J=7.2 Hz, 2H), 4.57-4.66 (m, 1H), 7.11 (dd, J=8.4, 2.0 Hz, 1H), 7.16 (d, J=2.0 Hz, 1H), 7.43 (d, J=8.4 Hz, 1H).

Compound 172D: LC-MS (ESI) m/z: non-ionizable compound under routine conditions used.

Compound 172E: LC-MS ESI (m/z): 276 [M+H]⁺; ¹H-NMR (CDCl₃, 400 MHz): δ (ppm) 1.39 (d, J=6.0 Hz, 6H), 4.38-4.41 (m, 1H), 4.57-4.66 (m, 1H), 7.07 (dd, J=8.4, 2.0 Hz, 1H), 7.13 (d, J=2.0 Hz, 1H), 7.46 (d, J=8.4 Hz, 1H).

Compound 172F: ¹H-NMR (CDCl₃, 400 MHz): δ (ppm) 1.38-1.40 (m, 6H), 3.26 (d, J=7.2 Hz, 1H), 3.84 (s, 3H), 4.49-4.64 (m, 2H), 6.98-7.08 (m, 2H), 7.41 (d, J=8.4 Hz, 1H).

Compound 172G hydrate: LC-MS ESI (m/z): 347 [M+H₂O+Na]⁺; ¹H-NMR (CDCl₃, 400 MHz): δ (ppm) 1.38 (d, J=6.0 Hz, 6H), 3.94 (s, 3H), 4.21 (s, 2H), 4.56-4.64 (m, 1H), 7.09 (dd, J=8.4, 1.6 Hz, 1H), 7.16 (d, J=1.6 Hz, 1H), 7.41 (d, J=8.4 Hz, 1H).

Compound 172H: LC-MS ESI (m/z): 291 [M−H]⁻.

Compound 1721 hydrochloride: LC-MS ESI (m/z): 294 [M+H]⁺.

Compound 172J: LC-MS ESI (m/z): 476 [M+H]⁺.

Compound 172K was used directly in next step without further purification. LC-MS ESI (m/z): 694 [M−55]⁺.

Compound 172 was separated with chiral HPLC to give Compound 172-1 and Compound 172-2.

Compound 172-1: LC-MS (ESI) m/z: 650 [M+H]⁺; ¹H-NMR (CD₃OD, 400 MHz): δ (ppm) 1.19-1.31 (m, 6H), 1.41-2.16 (m, 12H), 2.73-3.00 (m, 1H), 3.12-3.43 (m, 2H), 4.12-4.39 (m, 2H), 4.52-4.59 (m, 1H), 4.93-5.06 (m, 2H), 6.84-7.27 (m, 5H), 7.39-7.53 (m, 1H), 7.68-7.86 (m, 2H), 8.06-8.14 (m, 1H). Chiral separation condition: MeOH contained 0.5% NH₄OH; IC (4.6×150 mm, 5 μm); retention time: 4.55 minute.

Compound 172-2: LC-MS (ESI) m/z: 650 [M+H]⁺; ¹H-NMR (CD₃OD, 400 MHz): δ (ppm) 1.21-1.33 (m, 6H), 1.42-2.17 (m, 12H), 2.74-3.02 (m, 1H), 3.14-3.49 (m, 2H), 4.15-4.61 (m, 3H), 4.94-5.11 (m, 2H), 6.86-7.31 (m, 5H), 7.41-7.54 (m, 1H), 7.69-7.88 (m, 2H), 8.07-8.16 (m, 1H). Chiral separation condition: MeOH contained 0.5% NH₄OH; IC (4.6×150 mm, 5 μm); retention time: 3.83 minute.

### Example 173

### Synthesis of (R)-1-(3-(4′-chloro-[1,1′-biphenyl]-4-yl)-2-((6-(cyclopentyloxy) naphthalene)-2-sulfonamido)-3,3-difluoropropanoyl)piperidine-4-carboxamide (Compound 173-1), and (S)-1-(3-(4′-chloro-[1,1′-biphenyl]-4-yl)-2-((6-(cyclopentyloxy)naphthalene)-2-sulfonamido)-3,3-difluoropropanoyl)piperidine-4-carboxamide (Compound 173-2)

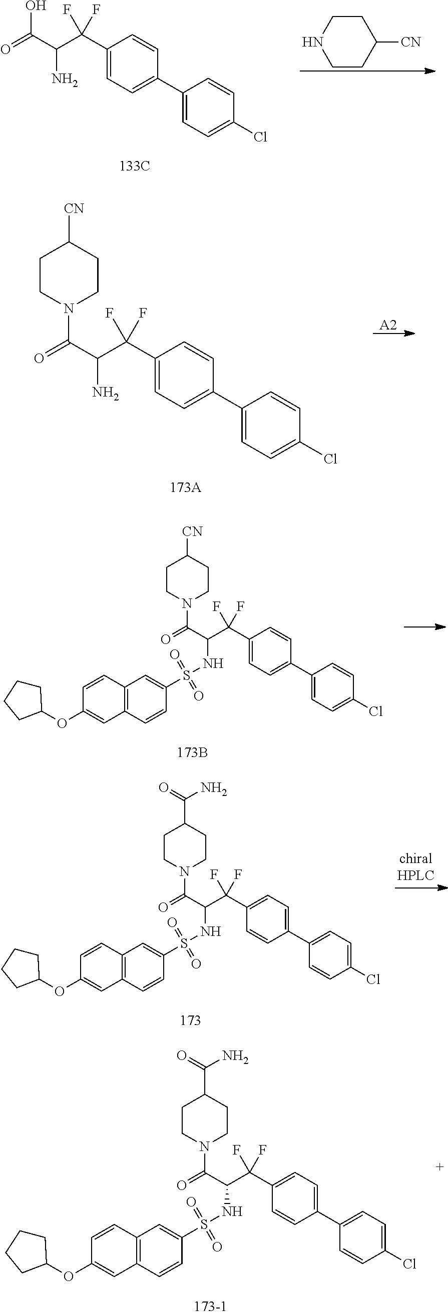

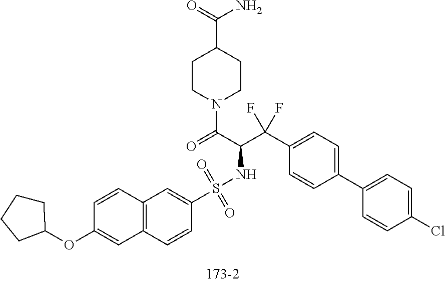 [SMILES: NC(=O)C1CCN(C(=O)[C@H](NS(=O)(=O)c2ccc3cc(OC4CCCC4)ccc3c2)C(F)(F)c2ccc(-c3ccc(Cl)cc3)cc2)CC1]

Compounds 173A and 173B were synthesized by employing the procedures described for Compound B4 and Compound 2D using piperidine-4-carbonitrile, Compounds 133C, 173A, and Compound A2 in lieu of Compounds B4-1, B2-6, B2, and Compound 2C.

Compound 173A: LC-MS (ESI) m/z: 404 [M+H]⁺.

Compound 173B: LC-MS (ESI) m/z: 678 [M+H]⁺.

To a mixture of Compound 173B (95 mg, 0.14 mmol) and potassium carbonate (58 mg, 0.42 mmol) in dimethylsulfoxide (5 mL) was added hydrogen peroxide (70 mg, 0.7 mmol, 30% aqueous solution). The reaction mixture was stirred at room temperature for 26 hours and filtered. The filtrate was diluted with water (30 mL) and extracted with EtOAc (20 mL×2). The combined organic extracts were dried over anhydrous sodium sulfate, filtered, and concentrated under reduced pressure. The residue was purified with preparative HPLC and chiral HPLC to give Compound 173-1 and Compound 173-2.

Compound 173-1: LC-MS (ESI) m/z: 696 [M+H]⁺; ¹H-NMR (CD₃OD, 400 MHz): δ (ppm) 1.16-2.61 (m, 15H), 2.78-2.96 (m, 1H), 3.89-4.16 (m, 2H), 4.94-5.06 (m, 1H), 7.09-7.18 (m, 2H), 7.43-7.54 (m, 8H), 7.58-7.60 (m, 1H), 7.72-7.75 (m, 1H), 7.80-7.86 (m, 1H), 8.15-8.18 (m, 1H). Chiral separation condition: Methanol contained 0.5% NH₄OH; (R,R)-Whelk-O1 (250×4.6 mm, 5 μm); retention time: 6.71 minute.

Compound 173-2: LC-MS (ESI) m/z: 696 [M+H]⁺; ¹H-NMR (CD₃OD, 400 MHz): δ (ppm) 1.16-2.61 (m, 15H), 2.78-2.96 (m, 1H), 3.89-4.16 (m, 2H), 4.94-5.06 (m, 1H), 7.09-7.18 (m, 2H), 7.43-7.54 (m, 8H), 7.58-7.60 (m, 1H), 7.72-7.75 (m, 1H), 7.80-7.86 (m, 1H), 8.15-8.18 (m, 1H). Chiral separation condition: Methanol contained 0.5% NH₄OH; (R,R)-Whelk-O1 (250×4.6 mm, 5 μm); retention time: 8.86 minute.

### Example 174

### Synthesis of (R)-1-(3-(4′-chloro-[1,1′-biphenyl]-4-yl)-2-((6-(cyclopentyloxy) naphthalene)-2-sulfonamido)-3,3-difluoropropanoyl)-N′-hydroxypiperidine-4-carboximidamide (Compound 174-1), and (S)-1-(3-(4′-chloro-[1,1′-biphenyl]-4-yl)-2-((6-(cyclopentyloxy)naphthalene)-2-sulfonamido)-3,3-difluoropropanoyl)-N′-hydroxypiperidine-4-carboximidamide (Compound 174-2)

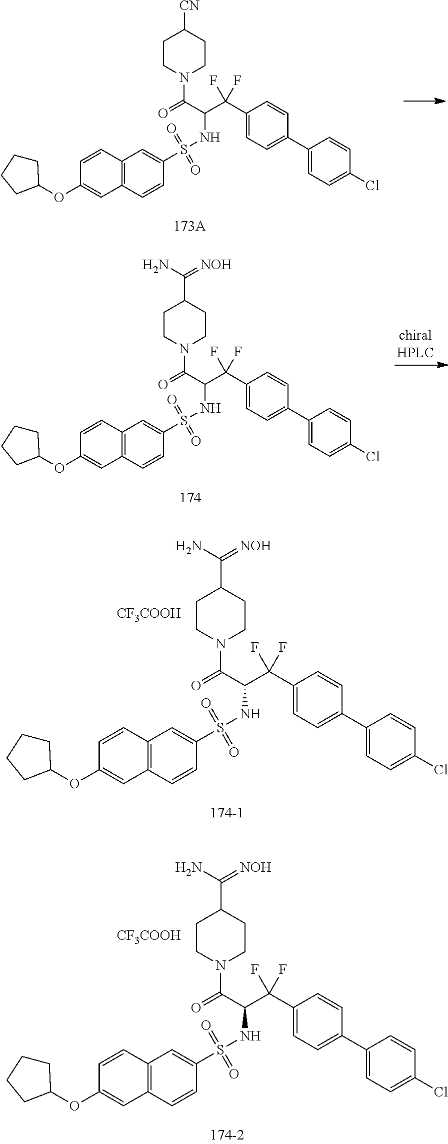

To a solution of Compound 173A (50 mg, 0.07 mmol) in ethanol (10 mL) was added NH₂OHHCl (5 mg). The reaction mixture was stirred at 70° C. for 16 hours and concentrated under reduced pressure. The residue was purified with preparative HPLC and chiral HPLC to give Compound 174-1 and Compound 174-2.

Compound 174-1: LC-MS (ESI) m/z: 711 [M+H]⁺; ¹H-NMR (CD₃OD, 400 MHz): δ (ppm) 1.41-1.91 (m, 13H), 2.43-2.65 (m, 1H), 2.86-3.05 (m, 1H), 4.16-4.64 (m, 3H), 4.92-5.00 (m, 1H), 6.82-7.26 (m, 3H), 7.29-7.42 (m, 8H), 7.49-7.59 (m, 1H), 7.51-7.72 (m, 1H), 7.93-8.01 (m, 1H). Chiral separation condition: n-Hexane/EtOH contained 0.1% DEA, 50/50; IC (4.6×250 mm, 5 μm); retention time: 11.58 minute.

Compound 174-2: LC-MS (ESI) m/z: 711 [M+H]⁺; ¹H-NMR (CD₃OD, 400 MHz): δ (ppm) 1.41-1.91 (m, 13H), 2.43-2.65 (m, 1H), 2.86-3.05 (m, 1H), 4.16-4.64 (m, 3H), 4.92-5.00 (m, 1H), 6.82-7.26 (m, 3H), 7.29-7.42 (m, 8H), 7.49-7.59 (m, 1H), 7.51-7.72 (m, 1H), 7.93-8.01 (m, 1H). Chiral separation condition: n-Hexane/EtOH contained 0.1% DEA, 50/50; IC (4.6×250 mm, 5 μm); retention time: 8.54 minute.

### Example 175

### Synthesis of (R)—N-(3-(4-aminopiperidin-1-yl)-1-(4′-chloro-[1,1′-biphenyl]-4-yl)-1,1-difluoro-3-oxopropan-2-yl)-2-(6-(cyclopentyloxy)benzo[b]thiophen-2-yl)-2-oxoacetamide trifluoroacetate (Compound 175-1), and (S)—N-(3-(4-aminopiperidin-1-yl)-1-(4′-chloro-[1,1′-biphenyl]-4-yl)-1,1-difluoro-3-oxopropan-2-yl)-2-(6-(cyclopentyloxy)benzo[b]thiophen-2-yl)-2-oxoacetamide trifluoroacetate (Compound 175-2)

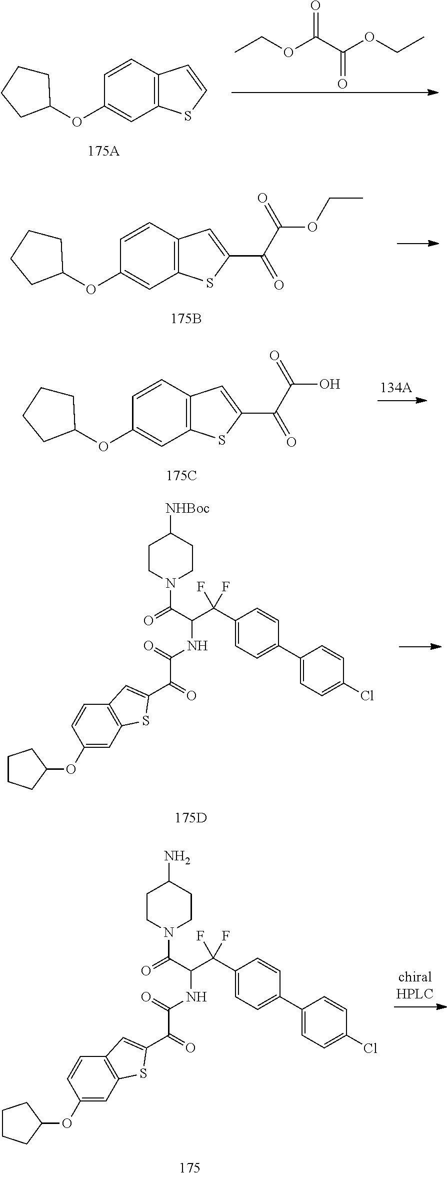

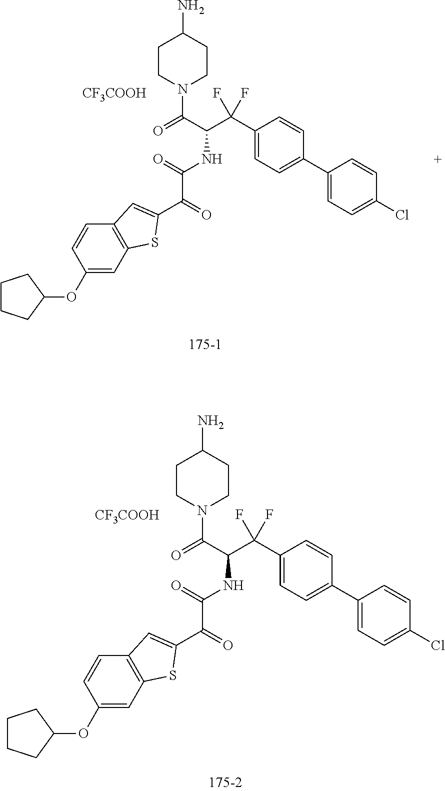 [SMILES: NC1CCN(C(=O)[C@@H](NC(=O)C(=O)c2cc3ccc(OC4CCCC4)cc3s2)C(F)(F)c2ccc(-c3ccc(Cl)cc3)cc2)CC1 ; "CF3COOH" ; NC1CCN(C(=O)[C@H](NC(=O)C(=O)c2cc3ccc(OC4CCCC4)cc3s2)C(F)(F)c2ccc(-c3ccc(Cl)cc3)cc2)CC1 ; "CF3COOH"]

Compounds 175B, 175C, 175D, and 175 were synthesized by employing the procedures described for Compounds 9B, 9C, Compound B4, and Compound 1 using Compounds 175A, 175B, 175C, 134A, and 175D in lieu of Compounds 9A, 9B, Compounds B2-6, B4-1, and Compound 1A.

Compound 175B: LC-MS: (ESI) m/z: 319 [M+H]⁺.

Compound 175C: LC-MS: (ESI) m/z: 289 [M−H]⁻.

Compound 175D: LC-MS (ESI) m/z: 788 [M+Na]⁺.

Compound 175 was separated with chiral HPLC to give Compound 175-1 and Compound 175-2.

Compound 175-1: LC-MS (ESI) m/z: 666 [M+H]⁺; ¹H-NMR (CD₃OD, 400 MHz): δ (ppm) 1.42-2.07 (m, 12H), 2.69-2.94 (m, 2H), 3.27-3.35 (m, 1H), 4.17-4.27 (m, 1H), 4.56-4.92 (m, 2H), 5.72-5.79 (m, 1H), 6.80-6.86 (m, 1H), 7.27-7.34 (m, 3H), 7.49-7.63 (m, 7H), 8.00-8.08 (m, 1H). Chiral separation condition: n-Hexane/EtOH contained 0.1% DEA, 40/60; IC (4.6×250 mm, 5 μm); retention time: 10.27 minute.

Compound 175-2: LC-MS (ESI) m/z: 666 [M+H]⁺; ¹H-NMR (CD₃OD, 400 MHz): δ (ppm) 1.42-2.07 (m, 12H), 2.69-2.94 (m, 2H), 3.27-3.35 (m, 1H), 4.17-4.27 (m, 1H), 4.56-4.92 (m, 2H), 5.72-5.79 (m, 1H), 6.80-6.86 (m, 1H), 7.27-7.34 (m, 3H), 7.49-7.63 (m, 7H), 8.00-8.08 (m, 1H). Chiral separation condition: n-Hexane/EtOH contained 0.1% DEA, 40/60; IC (4.6×250 mm, 5 μm); retention time: 8.53 minute.

### Example 176

### Synthesis of (R)—N-(1-(4′-chloro-[1,1′-biphenyl]-4-yl)-3-(4,6-dihydropyrrolo[3,4-c]pyrazol-5(1H)-yl)-1,1-difluoro-3-oxopropan-2-yl)-6-(cyclopentyloxy)naphthalene-2-sulfonamide (Compound 176-1), and (S)—N-(1-(4′-chloro-[1,1′-biphenyl]-4-yl)-3-(4,6-dihydropyrrolo[3,4-c]pyrazol-5(1H)-yl)-1,1-difluoro-3-oxopropan-2-yl)-6-(cyclopentyloxy)naphthalene-2-sulfonamide (Compound 176-2)

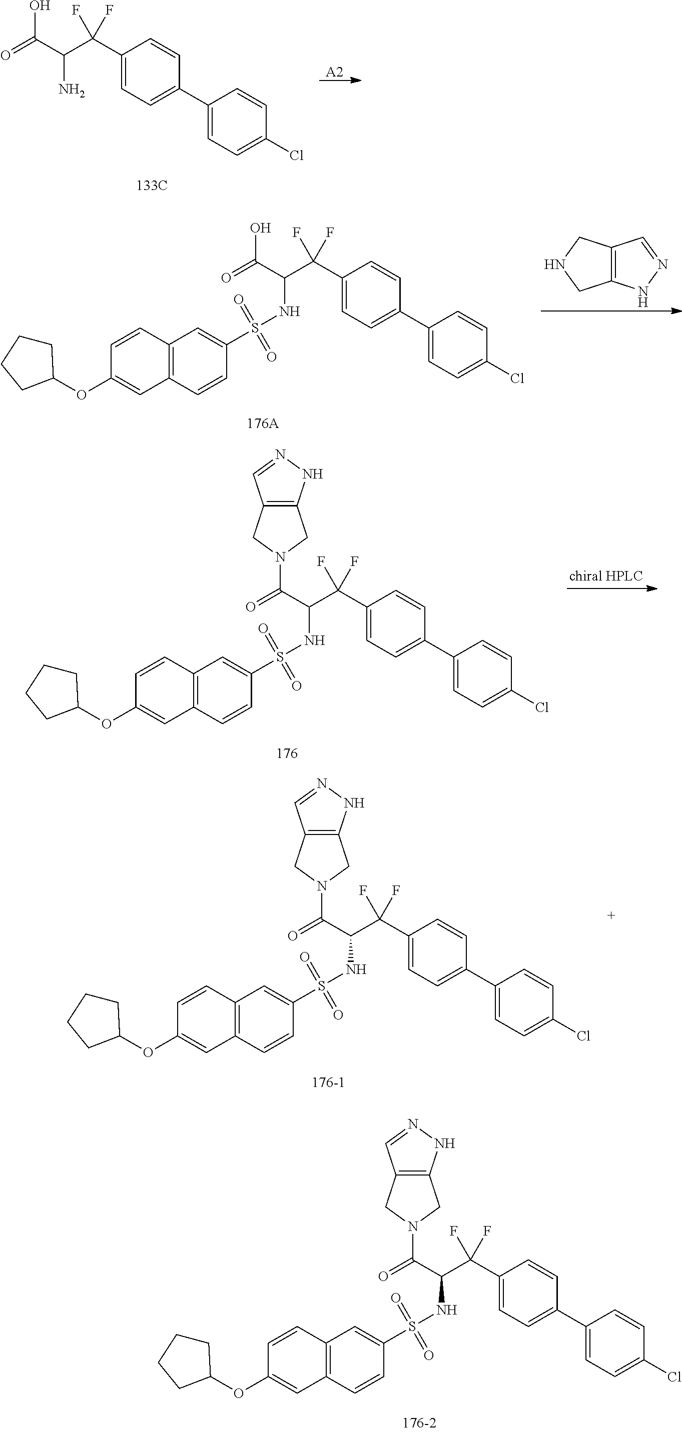

Compounds 176A and 176 were synthesized by employing the procedures described for Compound 1A and Compound B4 using Compound A2, Compounds 133C, 176A, and 1,4,5,6-tetrahydropyrrolo[3,4-c]pyrazole in lieu of Compounds A1, B2, B2-6, and B4-1.

Compound 176A: LC-MS (ESI) m/z: 586 [M+H]⁺.

Compound 176 was separated with chiral HPLC to yield Compound 176-1 and Compound 176-2.

Compound 176-1: LC-MS (ESI) m/z: 677 [M+H]⁺; ¹H-NMR (CD₃OD, 400 MHz): δ (ppm) 1.65-1.69 (m, 2H), 1.82-1.83 (m, 4H), 2.00-2.02 (m, 2H), 3.65-3.73 (m, 1H), 4.13-4.40 (m, 2H), 4.55-4.93 (m, 3H), 6.96-7.02 (m, 2H), 7.45-7.69 (m, 12H), 8.18-8.20 (m, 1H). Chiral separation condition: MeOH contained 0.5% NH₄OH; (R,R)-Whelk-O1 (4.6×250 mm, 5 μm); retention time: 5.74 minute.

Compound 176-2: LC-MS (ESI) m/z: 677 [M+H]⁺; ¹H-NMR (CD₃OD, 400 MHz): δ (ppm) 1.64-1.71 (m, 2H), 1.81-1.84 (m, 4H), 2.00-2.02 (m, 2H), 3.65-3.73 (m, 1H), 4.13-4.40 (m, 2H), 4.55-4.93 (m, 3H), 6.96-7.02 (m, 2H), 7.45-7.69 (m, 12H), 8.17-8.21 (m, 1H). Chiral separation condition: MeOH contained 0.5% NH₄OH; (R,R)-Whelk-O1 (4.6×250 mm, 5 μm); retention time: 7.47 minute.

### Example 177

### Synthesis of N-((2R)-3-(3-amino-8-azabicyclo[3.2.1]octan-8-yl)-1-(4-bromophenyl)-1,1-difluoro-3-oxopropan-2-yl)-6-cyclopropoxynaphthalene-2-sulfonamide trifluoroacetate (Compound 177-1), and N-((2S)-3-(3-amino-8-azabicyclo[3.2.1]octan-8-yl)-1-(4-bromophenyl)-1,1-difluoro-3-oxopropan-2-yl)-6-cyclopropoxynaphthalene-2-sulfonamide trifluoroacetate (Compound 177-2)

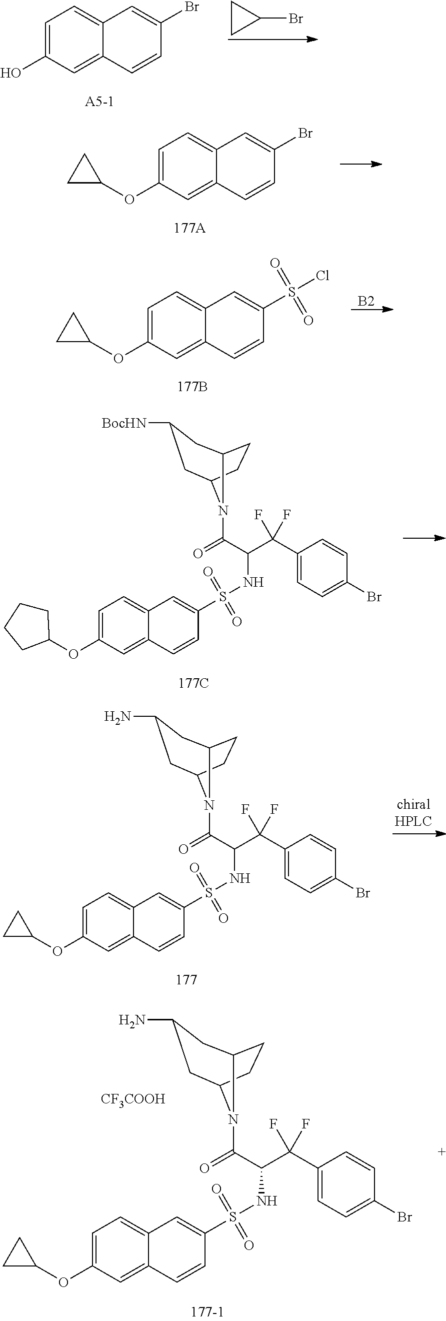

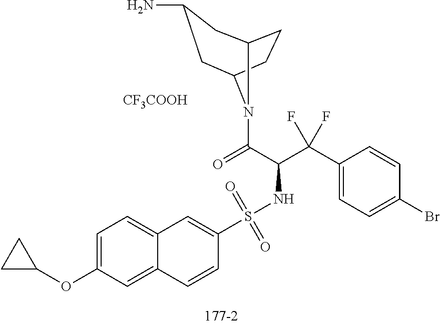 [SMILES: NC1CC2CCC(C1)N2C(=O)[C@H](NS(=O)(=O)c1ccc2cc(OC3CC3)ccc2c1)C(F)(F)c1ccc(Br)cc1 ; "CF3COOH"]

Compound 177A was synthesized by employing the procedure described for Compound A4-1 using bromocyclopropane and Compound A5-1 in lieu of (bromomethyl)cyclopentane and Compound 2A. ¹H-NMR (CDCl₃, 400 MHz): δ (ppm) 0.82-0.87 (m, 4H), 3.82-3.86 (m, 1H), 7.14 (dd, J=8.8, 2.4 Hz, 1H), 7.40 (d, J=2.4 Hz, 1H), 7.49 (dd, J=7.2, 1.6 Hz, 1H), 7.61-7.65 (m, 2H), 7.91 (d, J=2.0 Hz, 1H).

To a solution of Compound 177A (1 g, 3.8 mmol) in anhydrous THF (10 mL) was dropped a solution of n-butyl lithium in hexane (2.5 N, 1.8 mL, 4.56 mmol) at −78° C. under nitrogen. After stirred at −78° C. for 0.5 hour, the solution was slowly added into a mixture of sulfuryl dichloride (614 uL, 7.6 mmol) in anhydrous hexane (5 mL) via a syringe at −78° C. under nitrogen. The reaction mixture was stirred at −78° C. for 5 minutes, quenched with ice-water (30 mL), and extracted with ether (50 mL×2). The combined organic extracts were washed with brine (50 mL×3), dried over anhydrous sodium sulfate, filtered, and concentrated to give a crude product, which was purified by silica gel column chromatography to yield Compound 177B. ¹H-NMR (CDCl₃, 400 MHz): δ (ppm) 0.85-0.93 (m, 4H), 3.89-3.94 (m, 1H), 7.31 (dd, J=8.8, 2.4 Hz, 1H), 7.54 (d, J=3.6 Hz, 1H), 7.91-7.95 (m, 3H), 8.46-8.51 (m, 1H).

Compounds 177C and 177 were synthesized by employing the procedures described for Compounds 2D and 1 using Compounds 177B and 177C in lieu of Compounds 2C and 1A.

Compound 177C: LC-MS (ESI) m/z: 634 [M−Boc+H]⁺.

Compound 177 was separated with chiral HPLC to give Compound 177-1 and Compound 177-2.

Compound 177-1: LC-MS (ESI) m/z: 634 [M+H]⁺; ¹H-NMR (CD₃OD, 400 MHz): δ (ppm) 0.76-0.95 (m, 4H), 1.30-2.22 (m, 8H), 3.50-3.80 (m, 1H), 3.94-3.99 (m, 1H), 4.37-4.83 (m, 3H), 7.18 (t, J=9.2 Hz, 2H), 7.27-7.30 (m, 1H), 7.40-7.63 (m, 4H), 7.74-7.89 (m, 2H), 8.11-8.20 (m, 1H). Chiral separation condition: MeOH contained 0.5% Ammonia; (R,R)-Whelk-O1 (250×4.6 mm, 5 μm); retention time: 5.82 minute.

Compound 177-2: LC-MS (ESI) m/z: 634 [M+H]⁺; ¹H-NMR (CD₃OD, 400 MHz): δ (ppm) 0.76-0.93 (m, 4H), 1.30-2.22 (m, 8H), 3.50-3.78 (m, 1H), 3.94-4.00 (m, 1H), 4.37-4.82 (m, 3H), 7.16 (t, J=8.8 Hz, 2H), 7.26-7.31 (m, 1H), 7.40-7.63 (m, 4H), 7.74-7.89 (m, 2H), 8.11-8.20 (m, 1H). Chiral separation condition: MeOH contained 0.5% Ammonia; (R,R)-Whelk-O1 (250×4.6 mm, 5 μm); retention time: 7.18 minute.

### Example 178

### Synthesis of N—((R)-3-((3 S,4S)-4-amino-3-fluoropiperidin-1-yl)-1-(4′-chloro-[1,1′-biphenyl]-4-yl)-1,1-difluoro-3-oxopropan-2-yl)-6-(cyclopentyloxy)naphthalene-2-sulfonamide (Compound 178-1), N—((S)-3-((3 S,4S)-4-amino-3-fluoropiperidin-1-yl)-1-(4′-chloro-[1,1′-biphenyl]-4-yl)-1,1-difluoro-3-oxopropan-2-yl)-6-(cyclopentyloxy) naphthalene-2-sulfonamide (Compound 178-2), and N—((S)-3-((3R,4R)-4-amino-3-fluoropiperidin-1-yl)-1-(4′-chloro-[1,1′-biphenyl]-4-yl)-1,1-difluoro-3-oxopropan-2-yl)-6-(cyclopentyloxy)naphthalene-2-sulfonamide (Compound 178-3)

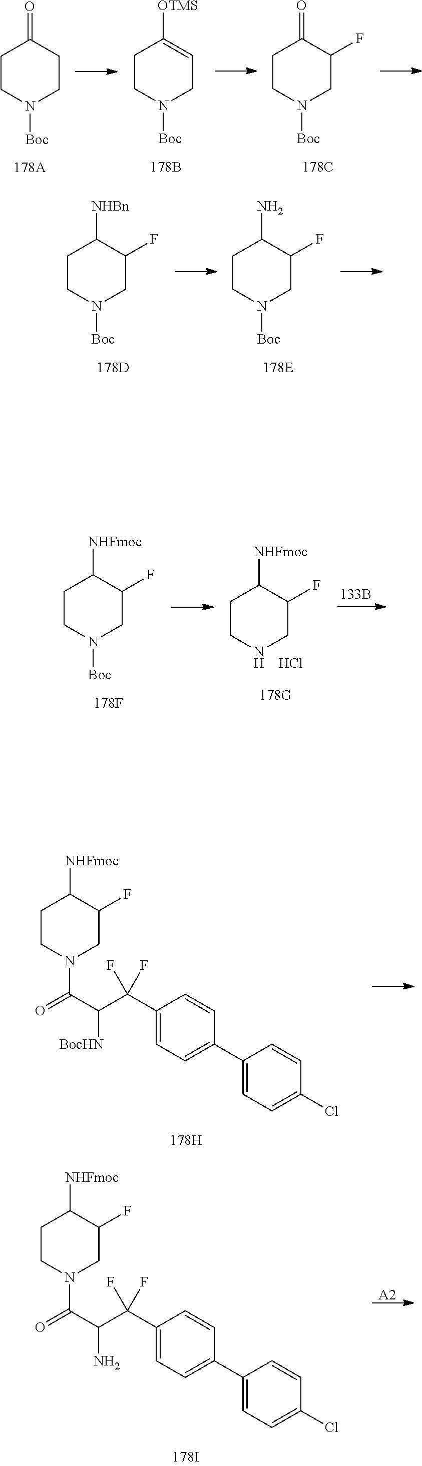

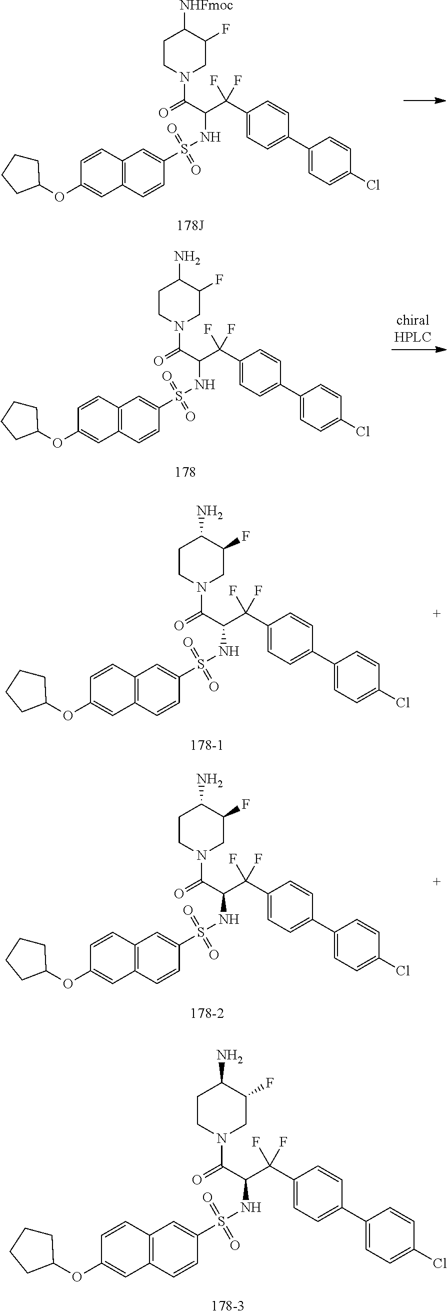

To a solution of Compound 178A (5 g, 25 mol) in DMF (10 mL) was add TMSCl (3.87 mL, 31 mmol) and TEA (8.5 mL). The reaction mixture was stirred at 80° C. under N₂ overnight. The reaction mixture was diluted with ether (100 mL), washed with cooled saturated NaHCO₃ solution (50 mL), dried over anhydrous sodium sulfate, filtered, and concentrated under reduced pressure. The residue was purified by silica gel column chromatography to furnish Compound 178B. LC-MS (ESI) m/z: 216 [M−55]⁺; ¹H-NMR (CDCl₃, 400 MHz): δ (ppm) 0.19 (s, 9H), 1.46 (s, 9H), 2.06-2.13 (m, 2H), 3.52 (t, J=4.2 Hz, 2H), 3.85-3.88 (m, 2H), 4.77-4.81 (m, 1H).

To a solution of Compound 178B (5.2 g, 19.16 mol) in acetonitrile (100 mL) was added Selectfluor (7.47 g, 21.07 mmol). The reaction mixture was stirred at room temperature for 2 hours, diluted with ethyl acetate (250 mL), washed with water (100 mL×2) and brine (100 mL), dried over anhydrous sodium sulfate, filtered, and concentrated to give Compound 178C. LC-MS (ESI) m/z: 162 [M−55]⁺.

Compounds 178D, 178E, 178F, 178G, 178H, 178I, and 178J were synthesized by employing the procedures described for Compounds B7-2, B7-3, B7-4, B7-5, B4, Compounds 6, and 1A using Compounds 178C, 178D, 178E, 178F, 178G, 133B, 178H, 178I, and Compound A2 in lieu of Compounds B7-1, B7-2, B7-3, B7-4, B4-1, B2-6, Compound 6C, Compound B2, and A1.

Compound 178D: LC-MS (ESI) m/z: 309 [M+H]⁺.

Compound 178E: LC-MS (ESI) m/z: 163 [M−55]⁺.

Compound 178F: LC-MS (ESI) m/z: 463 [M+Na]⁺.

Compound 178G: LC-MS (ESI) m/z: 341 [M+H]⁺.

Compound 178H: LC-MS (ESI) m/z: 756 [M+Na]⁺.

Compound 1781: LC-MS (ESI) m/z: 634 [M+H]⁺.

Compound 178J: LC-MS (ESI) m/z: non-ionizable compound under routine conditions used.

To a solution of Compound 178J (300 mg, 0.33 mmol) in dichloromethane (10 mL) was added piperidine (1 mL). The mixture was stirred at ambient temperature overnight. The reaction mixture was diluted with dichloromethane (100 mL), washed with saturated NH₄C₁ solution (100 mL) and brine (100 mL), dried over anhydrous sodium sulfate, filtered, and concentrated under reduced pressure. The residue was purified with reverse phase chromatography using eluent (methanol in water, from 0% to 100% v/v) to afford Compound 178, which was further separated with chiral HPLC to give Compound 178-1, Compound 178-2, and Compound 178-3.

Compound 178-1: LC-MS (ESI) m/z: 686 [M+H]⁺; ¹H-NMR (CD₃OD, 400 MHz): δ (ppm) 1.70-2.12 (m, 11H), 2.42-2.87 (m, 1H), 3.02-3.15 (m, 1H), 3.41-3.67 (m, 1H), 4.05-4.38 (m, 1H), 4.64-4.76 (m, 1H), 5.04-5.36 (m, 2H), 6.91-7.25 (m, 2H), 7.32-7.84 (m, 11H), 8.03-8.13 (m, 1H). Chiral separation condition: n-hexane/EtOH contained 0.1% DEA, 50/50; (R,R)-Whelk-O1 (4.6×250 mm, 5 μm); retention time: 15.83 minute.

Compound 178-2: LC-MS (ESI) m/z: 686 [M+H]⁺; ¹H-NMR (CD₃OD, 400 MHz): δ (ppm) 1.60-1.86 (m, 8H), 2.00-2.11 (m, 4H), 3.00-3.15 (m, 1H), 3.48-3.66 (m, 1H), 4.44-4.73 (m, 2H), 5.15-5.36 (m, 2H), 6.90-7.24 (m, 2H), 7.35-7.80 (m, 11H), 8.01-8.08 (m, 1H). Chiral separation condition: n-hexane/EtOH contained 0.1% DEA, 50/50; (R,R)-Whelk-O1 (4.6×250 mm, 5 μm); retention time: 15.23 minute.

Compound 178-3: LC-MS (ESI) m/z: 686 [M+H]⁺; ¹H-NMR (CD₃OD, 400 MHz): δ (ppm) 1.69-1.85 (m, 8H), 21.98-2.43 (m, 4H), 3.01-3.14 (m, 1H), 3.41-3.75 (m, 1H), 4.44-4.74 (m, 2H), 5.07-5.22 (m, 2H), 6.90-7.12 (m, 2H), 7.22-7.80 (m, 11H), 8.02-8.08 (m, 1H). Chiral separation condition: n-hexane/EtOH contained 0.1% DEA, 50/50; (R,R)-Whelk-O1 (4.6×250 mm, 5 μm); retention time: 13.08 minute.

### Example 179

### Synthesis of N—((R)-1-(4′-chloro-[1,1′-biphenyl]-4-yl)-1,1-difluoro-3-((3aR,6aS)-hexahydropyrrolo[3,4-c]pyrrol-2(1H)-yl)-3-oxopropan-2-yl)-4-(cyclohexylmethoxy) benzenesulfonamide trifluoroacetate (Compound 179-1), and N—((S)-1-(4′-chloro-[1,1′-biphenyl]-4-yl)-1,1-difluoro-3-((3aR,6aS)-hexahydropyrrolo[3,4-c]pyrrol-2(1H)-yl)-3-oxopropan-2-yl)-4-(cyclohexylmethoxy)benzenesulfonamide trifluoroacetate (Compound 179-2)

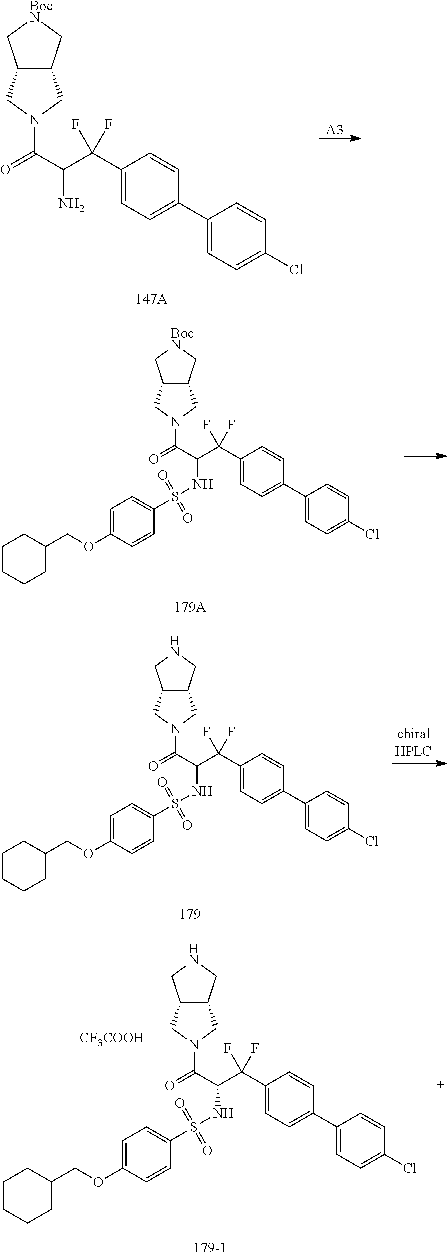 [SMILES: CC(C)(C)OC(=O)N1C[C@@H]2CN(C(=O)C(N)C(F)(F)c3ccc(-c4ccc(Cl)cc4)cc3)C[C@@H]2C1 ; "A3" ; CC(C)(C)OC(=O)N1C[C@@H]2CN(C(=O)C(NS(=O)(=O)c3ccc(OCC4CCCCC4)cc3)C(F)(F)c3ccc(-c4ccc(Cl)cc4)cc3)C[C@@H]2C1 ; O=C(C(NS(=O)(=O)c1ccc(OCC2CCCCC2)cc1)C(F)(F)c1ccc(-c2ccc(Cl)cc2)cc1)N1C[C@H]2CNC[C@H]2C1 ; O=C([C@@H](NS(=O)(=O)c1ccc(OCC2CCCCC2)cc1)C(F)(F)c1ccc(-c2ccc(Cl)cc2)cc1)N1C[C@H]2CNC[C@H]2C1 ; "CF3COOH"]

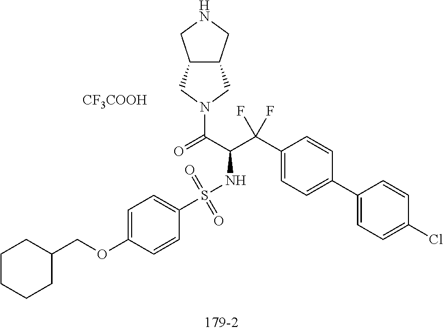 [SMILES: O=C([C@H](NS(=O)(=O)c1ccc(OCC2CCCCC2)cc1)C(F)(F)c1ccc(-c2ccc(Cl)cc2)cc1)N1C[C@H]2CNC[C@H]2C1 ; "CF3COOH"]

Compounds 179A and 179 were synthesized by employing the procedures described for Compounds 2D and 6 using Compound 147A, Compound A3, and Compound 179A in lieu of Compound B2, Compounds 2C, and 6C.

Compound 179A: LC-MS (ESI) m/z: 758 [M+H]⁺.

Compound 179 was separated with chiral HPLC to furnish Compound 179-1 and Compound 179-2.

Compound 179-1: LC-MS (ESI) m/z: 658 [M+H]⁺; ¹H-NMR (CD₃OD, 400 MHz): δ (ppm) 0.94-0.99 (m, 2H), 1.26-1.78 (m, 11H), 2.97-3.18 (m, 4H), 3.58-3.64 (m, 4H), 3.78-3.88 (m, 2H), 4.59-4.65 (m, 1H), 6.73-6.83 (m, 2H), 7.40-7.59 (m, 8H), 7.65-7.67 (m, 2H). Chiral separation condition: n-Hexane/EtOH contained 0.1% DEA, 50/50; IA (4.6×250 mm, 5 μm); retention time: 11.92 minute.

Compound 179-2: LC-MS (ESI) m/z: 658 [M+H]⁺; ¹H-NMR (CD₃OD, 400 MHz): δ (ppm) 0.92-0.97 (m, 2H), 1.13-1.68 (m, 11H), 2.99-3.12 (m, 4H), 3.58-3.64 (m, 4H), 3.78-3.88 (m, 2H), 4.59-4.65 (m, 1H), 6.61-6.73 (m, 2H), 7.31-7.49 (m, 8H), 7.55-7.58 (m, 2H). Chiral separation condition: n-Hexane/EtOH contained 0.1% DEA, 50/50; IA (4.6×250 mm, 5 μm); retention time: 17.54 minute.

### Example 180

### Synthesis of N-((2R)-3-((2S)-2-amino-7-azabicyclo[2.2.1]heptan-7-yl)-1-(4′-chloro-[1,1′-biphenyl]-4-yl)-1,1-difluoro-3-oxopropan-2-yl)-6-(cyclopentyloxy)naphthalene-2-sulfonamide (Compound 180-1), and N-((2S)-3-((2S)-2-amino-7-azabicyclo[2.2.1]heptan-7-yl)-1-(4′-chloro-[1,1′-biphenyl]-4-yl)-1,1-difluoro-3-oxopropan-2-yl)-6-(cyclopentyloxy)naphthalene-2-sulfonamide (Compound 180-2), N-((2S)-3-((2R)-2-amino-7-azabicyclo[2.2.1]heptan-7-yl)-1-(4′-chloro-[1,1′-biphenyl]-4-yl)-1,1-difluoro-3-oxopropan-2-yl)-6-(cyclopentyloxy)naphthalene-2-sulfonamide (Compound 180-3), and N-((2R)-3-((2R)-2-amino-7-azabicyclo[2.2.1]heptan-7-yl)-1-(4′-chloro-[1,1′-biphenyl]-4-yl)-1,1-difluoro-3-oxopropan-2-yl)-6-cyclopentyloxy) naphthalene-2-sulfonamide (Compound 180-4)

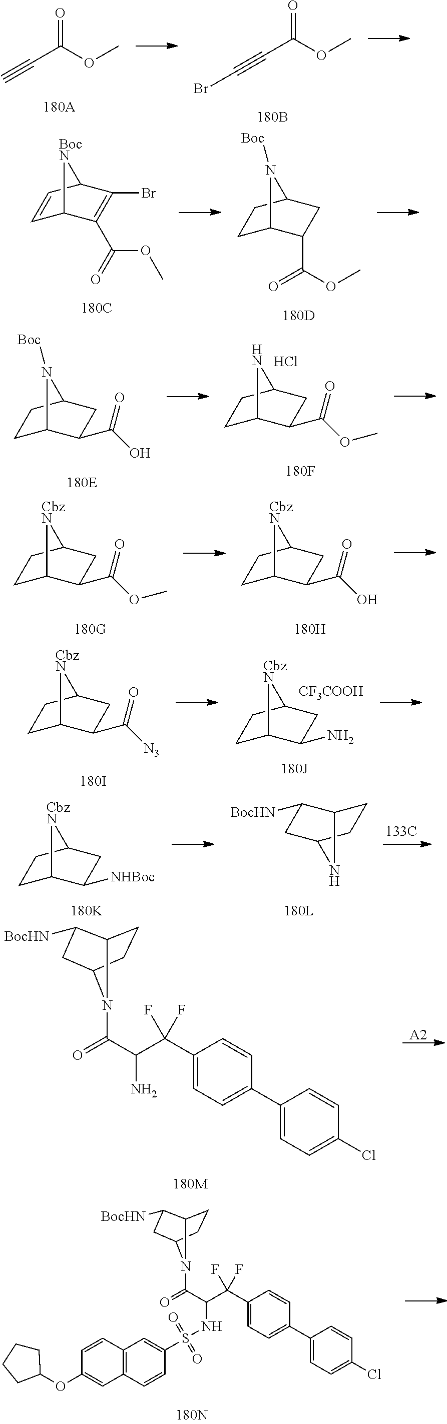

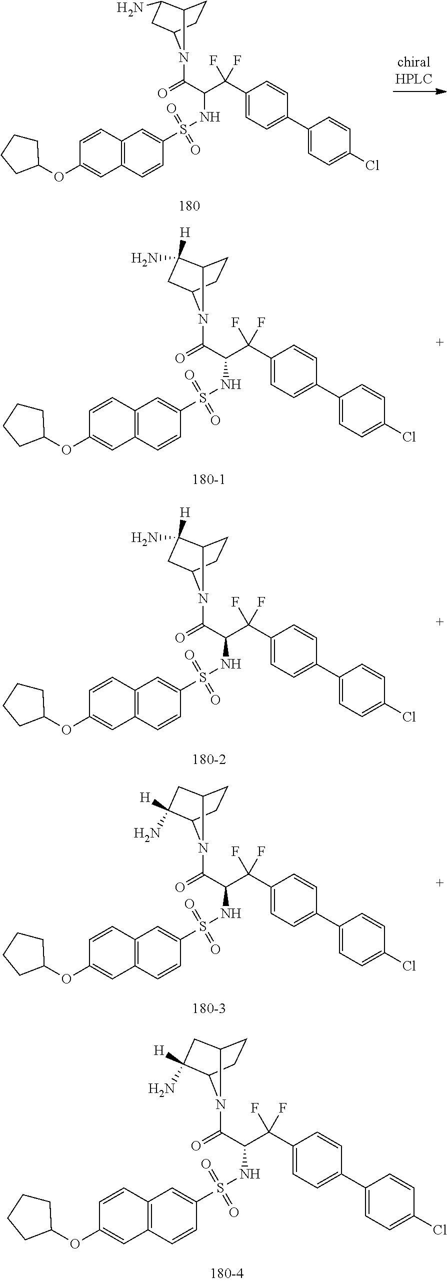 [SMILES: NC1CC2CCC1N2C(=O)C(NS(=O)(=O)c1ccc2cc(OC3CCCC3)ccc2c1)C(F)(F)c1ccc(-c2ccc(Cl)cc2)cc1 ; N[C@H]1CC2CCC1N2C(=O)[C@@H](NS(=O)(=O)c1ccc2cc(OC3CCCC3)ccc2c1)C(F)(F)c1ccc(-c2ccc(Cl)cc2)cc1 ; N[C@H]1CC2CCC1N2C(=O)[C@H](NS(=O)(=O)c1ccc2cc(OC3CCCC3)ccc2c1)C(F)(F)c1ccc(-c2ccc(Cl)cc2)cc1 ; N[C@@H]1CC2CCC1N2C(=O)[C@H](NS(=O)(=O)c1ccc2cc(OC3CCCC3)ccc2c1)C(F)(F)c1ccc(-c2ccc(Cl)cc2)cc1 ; N[C@@H]1CC2CCC1N2C(=O)[C@@H](NS(=O)(=O)c1ccc2cc(OC3CCCC3)ccc2c1)C(F)(F)c1ccc(-c2ccc(Cl)cc2)cc1]

To a solution of methyl propiolate, Compound 180A, (5.0 g, 59.5 mmol) in acetone (170 mL) was added NBS (12.2 g, 68.4 mmol) and AgNO₃ (1.0 g, 5.95 mmol) at room temperature. The reaction mixture was stirred at room temperature overnight and filtered. The filtrate was concentrated under reduced pressure. The residue was purified by silica gel column chromatography to furnish Compound 180B. ¹H-NMR (CDCl₃, 400 MHz): δ (ppm) 3.75 (s, 3H).

A mixture of Compound 180B (4.6 g, 28.4 mmol) and tert-butyl 1H-pyrrole-1-carboxylate (23.7 g, 142 mmol) was stirred at 95° C. for 30 hours. The resulting mixture cooled down to room temperature and purified by silica gel column chromatography to afford Compound 180C. ¹H-NMR (CDCl₃, 400 MHz): δ (ppm) 1.42 (s, 9H), 3.80 (s, 3H), 5.13-5.18 (m, 1H), 5.50 (s, 1H), 7.09-7.14 (m, 2H).

To a solution of Compound 180C (4.2 g, 12.8 mmol) and triethylamine (1.42 g, 14.0 mmol) in methanol (100 mL) was added 10% Pd/C (420 mg). The resulting mixture was stirred under hydrogen at room temperature overnight and filtered with Celite. The filtration was concentrated under reduced pressure. The crude product was diluted with dichloromethane (50 mL) and washed with water (30 mL×2). The organic layer was dried over anhydrous sodium sulfate and concentrated to give Compound 180D. ¹H-NMR (CDCl₃, 400 MHz): δ (ppm) 1.48 (s, 9H), 1.48-1.90 (m, 6H), 3.02-3.08 (m, 1H), 3.68 (s, 3H), 4.07-4.21 (m, 2H).

To a solution of Compound 180D (15.3 g, 60 mmol) in methanol (100 mL) was added sodium methoxide (8.1 g, 150 mmol). The resulting mixture was refluxed for 5 hours, cooled to room temperature, and filtered through Celite. The filtrate was diluted with water (20 mL), adjusted pH to 2 with aqueous HCl solution (2 N), and extracted with dichloromethane (100 mL×2). The combined organic layers was washed with water (100 mL), dried over anhydrous sodium sulfate, filtered, and concentrated to give a crude oil. The crude product was diluted with methanol (50 mL) and a white solid was precipitated. After filtered, the solid was washed with methanol (5 mL) and dried to furnish Compound 180E. LC-MS (ESI) m/z: 505 [2M+Na]⁺; ¹H-NMR (CDCl₃, 400 MHz): δ (ppm) 1.42 (s, 9H), 1.42-1.52 (m, 2H), 1.61-1.66 (m, 1H), 1.77-1.85 (m, 2H), 2.23-2.26 (m, 1H), 2.58-2.61 (m, 1H), 4.31 (s, 1H), 4.56 (d, J=3.6 Hz, 1H).

To a solution of Compound 180E (3.5 g, 14.5 mmol) in methanol (40 mL) was added sulfurous dichloride (3.2 mL, 43.5 mmol) at 0° C. The reaction mixture was refluxed for 2 hours. The reaction mixture was concentrated to furnish Compound 180F. LC-MS (ESI) m/z: 192 [M+H]⁺; ¹H-NMR (CD₃OD, 400 MHz): δ (ppm) 1.79-1.84 (m, 2H), 1.98-2.01 (m, 2H), 2.13-2.23 (m, 2H), 3.00-3.05 (m, 1H), 3.75 (s, 3H), 4.26 (t, J=4.0 Hz, 1H), 4.48 (m, 1H).

To a stirred solution of Compound 180F (2.77 g, 14.5 mmol) in dichloromethane (10 mL) was added N,N-diisopropylethylamine (8.5 mL, 50.75 mmol) at 0° C. and stirred at 0° C. for 10 min. To the reaction mixture was added benzyl carbonochloridate (2.96 g, 17.4 mmol) dropwise. The reaction mixture was stirred at room temperature for 1 hour, poured into water (50 mL), and extracted with dichloromethane (100 mL×2). The combined organic extracts were washed with water (100 mL×2) and brine (200 mL), dried over sodium sulfate, filtered, and concentrated under reduced pressure. The residue was purified by silica gel column chromatography to furnish Compound 180G. LC-MS (ESI) m/z: 290 [M+H]⁺; ¹H-NMR (DMSO-d₆, 400 MHz): δ (ppm) 1.39-1.68 (m, 5H), 1.99-2.05 (m, 1H), 2.71-2.74 (m, 1H), 3.54 (s, 3H), 4.24 (t, J=4.4 Hz, 1H), 4.41 (d, J=4.0 Hz, 1H), 4.98-5.05 (m, 2H), 7.29-7.39 (m, 5H).

To a solution of Compound 180G (5 g, 17.3 mmol) in methanol (50 mL) and water (20 mL) was added LiOHH₂O (1.45 mg, 34.6 mmol). The reaction mixture was stirred at room temperature for 2 hours, diluted with water (100 mL), and extracted with ethyl acetate (100 mL×2). The aqueous phase was acidified to pH 2 with aqueous HCl solution (2 N) and extracted with ethyl acetate (100 mL×3). The combined organic extracts were dried over anhydrous sodium sulfate, filtered, and concentrated to give a crude Compound 180H. LC-MS (ESI) m/z: 276 [M+H]⁺. ¹H-NMR (CDCl₃, 400 MHz): δ (ppm) 1.40-1.54 (m, 2H), 1.65-1.70 (m, 1H), 1.78-1.84 (m, 2H), 2.21-2.26 (m, 1H), 2.59-2.63 (m, 1H), 4.41-4.42 (m, 1H), 4.65-4.66 (m, 1H), 5.01-5.12 (m, 2H), 7.26-7.37 (m, 5H).

To a solution of Compound 180H (5 g, 18.2 mmol) in THF (30 mL) at 0° C. was added triethylamine (5.2 mL, 36.4 mmol), followed by addition of isobutyl chloroformate (2.7 mL, 21.8 mmol). After the reaction mixture was stirred at 0° C. for 1 hour, a solution of sodium azide (3.55 g, 54.6 mmol) in water (10 mL) was added. The reaction mixture was stirred at 0° C. for 30 minutes, diluted with water (50 mL), and extracted with ethyl acetate (50 mL×2). The combined organic extracts were washed with saturated sodium bicarbonate solution (50 mL) and brine (50 mL), dried over anhydrous sodium sulfate, filtered, and concentrated to give Compound 1801. LC-MS (ESI) m/z: 301 [M+H]⁺.

A solution of Compound 1801 (2 g, 6.67 mmol) in aqueous HCl solution (4 N, 40 mL) was stirred at 100° C. for 2 hours and concentrated under reduced pressure. The residue was purified with preparative HPLC to furnish Compound 180J. LC-MS (ESI) m/z: 247 [M+H]⁺.

A mixture of Compound 180J (350 mg, 0.97 mmol), di-tert-butyl dicarbonate (240 mg, 1.1 mmol), and triethylamine (0.78 mL, 6 mmol) in dichloromethane (10 mL) was stirred at room temperature for 3 hours. The mixture was diluted with ethyl acetate (100 mL), washed with water (50 mL×2) and brine (50 mL×2), dried over anhydrous sodium sulfate, filtered, and concentrated to give Compound 180K. LC-MS (ESI) m/z: 347 [M+H]⁺; ¹H-NMR (CDCl₃, 400 MHz): δ (ppm) 1.42 (s, 9H), 1.61-1.75 (m, 5H), 1.90-1.96 (m, 1H), 3.71-3.75 (m, 1H), 4.20 (t, J=4.8 Hz, 1H), 4.32 (m, 1H), 4.67 (brs, 1H), 5.10-5.12 (m, 2H), 7.34-7.37 (m, 5H).

Compounds 180L, 180M, 180N, and 181 were synthesized by employing the procedures described for Compounds B7-3, B4, Compounds 2D, and 1 using Compounds 180K, 180L, 133C, 180M, Compound A2, and Compound 180N in lieu of Compounds B7-2, B4-1, B2-6, B2, Compounds 2C, and 1A.

Compound 180L: LC-MS (ESI) m/z: 213 [M+H]⁺.

Compound 180M: LC-MS (ESI) m/z: 506 [M+H]⁺.

Compound 180N: LC-MS (ESI) m/z: 802 [M+Na]⁺.

Compound 180 was separated with chiral HPLC to afford Compound 180-1, Compound 180-2, Compound 180-3, and Compound 180-4.

Compound 180-1: LC-MS (ESI) m/z: 680 [M+H]⁺; ¹H-NMR (CD₃OD, 400 MHz): δ (ppm) 1.19-1.93 (m, 14H), 2.80-3.03 (m, 1H), 4.06-4.18 (m, 1H), 4.39-4.72 (m, 3H), 6.76-7.11 (m, 3H), 7.22-7.69 (m, 10H), 7.93-8.03 (m, 1H). Chiral separation condition: MeOH contained 0.5% NH₄OH; OJ-H (250×4.6 mm, 5 μm); retention time: 3.00 minute.

Compound 180-2: LC-MS (ESI) m/z: 680 [M+H]⁺; ¹H-NMR (CD₃OD, 400 MHz): δ (ppm) 1.30-2.11 (m, 14H), 2.86-2.97 (m, 1H), 4.26-4.68 (m, 4H), 6.83-6.99 (m, 2H), 7.18-7.68 (m, 11H), 7.93-7.99 (m, 1H). Chiral separation condition: MeOH contained 0.5% NH₄OH; OJ-H (250×4.6 mm, 5 μm); retention time: 5.31 minute.

Compound 180-3: LC-MS (ESI) m/z: 680 [M+H]⁺; ¹H-NMR (CD₃OD, 400 MHz): δ (ppm) 1.33-2.11 (m, 14H), 2.79-3.01 (m, 1H), 4.07-4.58 (m, 4H), 6.75-7.68 (m, 13H), 7.93-8.01 (m, 1H). Chiral separation condition: MeOH contained 0.5% NH₄OH; OJ-H (250×4.6 mm, 5 μm); retention time: 3.47 minute.

Compound 180-4: LC-MS (ESI) m/z: 680 [M+H]⁺; ¹H-NMR (CD₃OD, 400 MHz): δ (ppm) 1.50-2.11 (m, 14H), 3.48-3.50 (m, 1H), 4.05-4.62 (m, 4H), 6.81-7.93 (m, 14H). Chiral separation condition: MeOH contained 0.5% NH₄OH; OJ-H (250×4.6 mm, 5 μm); retention time: 4.58 minute.

### Example 181

### Synthesis of (R)—N-(3-(4-aminopiperidin-1-yl)-1-(4′-chloro-[1,1′-biphenyl]-3-yl)-1,1-difluoro-3-oxopropan-2-yl)-4-(cyclohexylmethoxy)benzenesulfonamide trifluoroacetate (Compound 181-1), and (S)—N-(3-(4-aminopiperidin-1-yl)-1-(4′-chloro-[1,1′-biphenyl]-3-yl)-1,1-difluoro-3-oxopropan-2-yl)-4-(cyclohexylmethoxy) benzenesulfonamide trifluoroacetate (Compound 181-2)

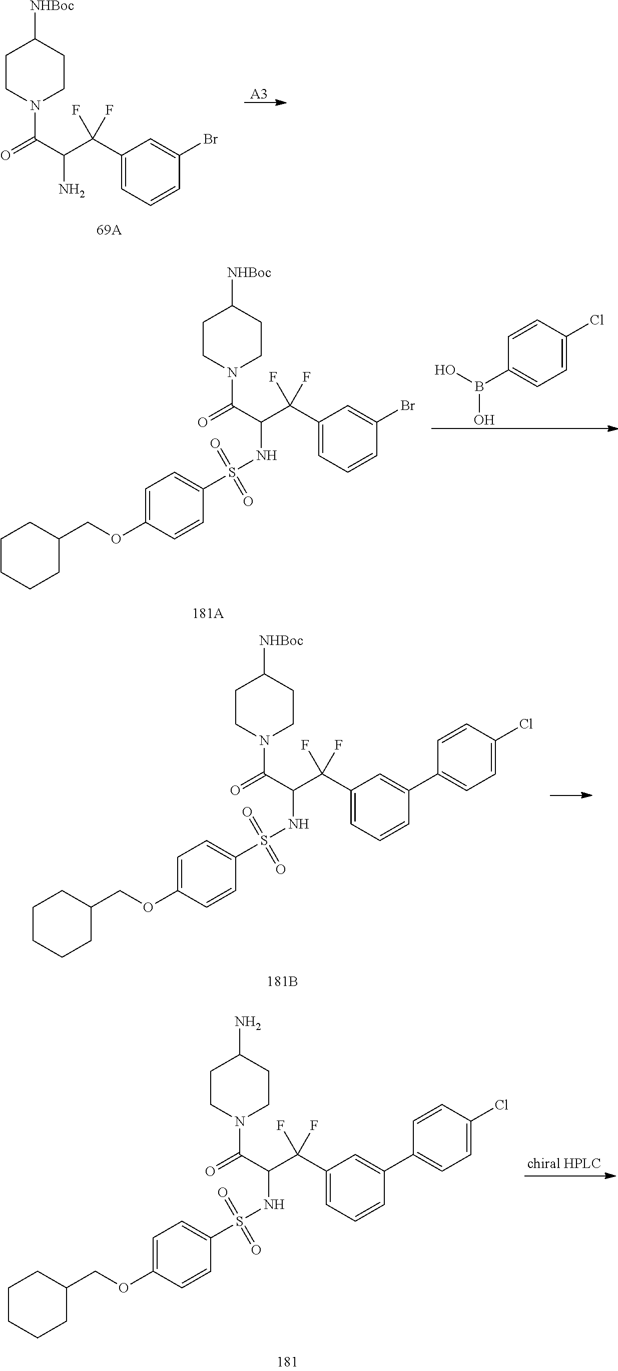 [SMILES: CC(C)(C)OC(=O)NC1CCN(C(=O)C(N)C(F)(F)c2cccc(Br)c2)CC1 ; "A3" ; CC(C)(C)OC(=O)NC1CCN(C(=O)C(NS(=O)(=O)c2ccc(OCC3CCCCC3)cc2)C(F)(F)c2cccc(Br)c2)CC1 ; OB(O)c1ccc(Cl)cc1 ; CC(C)(C)OC(=O)NC1CCN(C(=O)C(NS(=O)(=O)c2ccc(OCC3CCCCC3)cc2)C(F)(F)c2cccc(-c3ccc(Cl)cc3)c2)CC1 ; NC1CCN(C(=O)C(NS(=O)(=O)c2ccc(OCC3CCCCC3)cc2)C(F)(F)c2cccc(-c3ccc(Cl)cc3)c2)CC1]

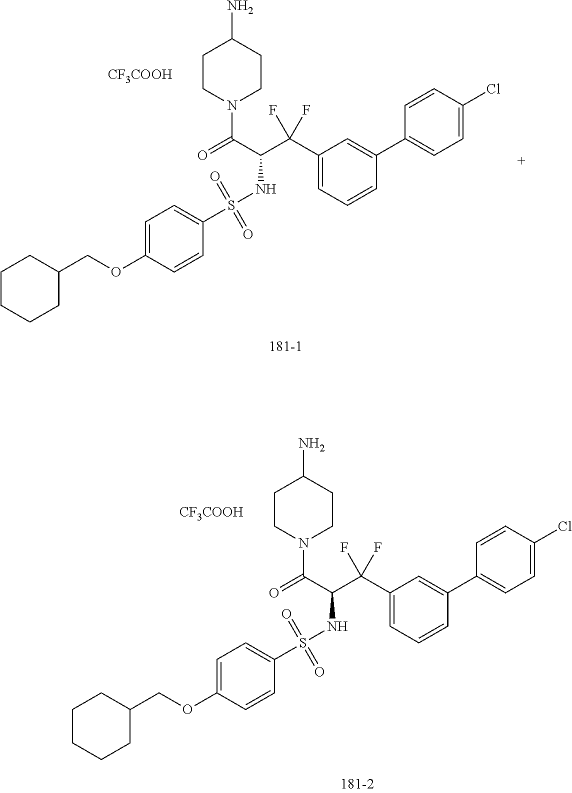 [SMILES: NC1CCN(C(=O)[C@@H](NS(=O)(=O)c2ccc(OCC3CCCCC3)cc2)C(F)(F)c2cccc(-c3ccc(Cl)cc3)c2)CC1 ; "CF3COOH" ; NC1CCN(C(=O)[C@H](NS(=O)(=O)c2ccc(OCC3CCCCC3)cc2)C(F)(F)c2cccc(-c3ccc(Cl)cc3)c2)CC1 ; "CF3COOH"]

Compounds 181A, 181B, and 181 were synthesized by employing the procedures described for Compounds 2D, 14C, and 1 using Compound A3, Compounds 69A, 181A, 4-chlorophenylboronic acid, and 181B in lieu of Compound 2C, Compound B2, Compounds 14A, 14B, and A1.

Compound 181A: LC-MS (ESI) m/z: 658 [M−55]⁺.

Compound 181B: LC-MS (ESI) m/z: 690 [M−55]⁺.

Compound 181 was separated with chiral HPLC to furnish Compound 181-1 and Compound 181-2.

Compound 181-1: LC-MS (ESI) m/z: 646 [M+H]⁺; ¹H-NMR (CD₃OD, 400 MHz): δ (ppm) 1.02-1.10 (m, 2H), 1.23-1.38 (m, 3H), 1.45-1.85 (m, 8H), 2.00-2.15 (m, 2H), 2.47-2.81 (m, 1H), 3.10-3.22 (m, 1H), 3.36-3.44 (m, 1H), 3.61 (d, J=6.4 Hz, 1H), 3.71 (d, J=6.0 Hz, 1H), 4.20-4.34 (m, 1H), 4.40-4.60 (m, 1H), 4.97-5.08 (m, 1H), 6.76 (d, J=8.8 Hz, 1H), 6.86 (d, J=9.2 Hz, 1H), 7.44-7.65 (m, 9H), 7.68-7.74 (m, 1H). Chiral separation condition: MeOH contained 0.2% Methanol Ammonia); OJ-H (4.6×250 mm, 5 m); retention time: 2.53 minute.

Compound 181-2: LC-MS (ESI) m/z: 646 [M+H]⁺; ¹H-NMR (CD₃OD, 400 MHz): δ (ppm) 1.02-1.11 (m, 2H), 1.23-1.35 (m, 3H), 1.38-1.85 (m, 8H), 1.99-2.15 (m, 2H), 2.47-2.81 (m, 1H), 3.10-3.22 (m, 1H), 3.36-3.44 (m, 1H), 3.61 (d, J=6.0 Hz, 1H), 3.71 (d, J=6.4 Hz, 1H), 4.20-4.34 (m, 1H), 4.40-4.61 (m, 1H), 4.98-5.08 (m, 1H), 6.76 (d, J=8.8 Hz, 1H), 6.85 (d, J=8.8 Hz, 1H), 7.44-7.65 (m, 9H), 7.68-7.74 (m, 1H). Chiral separation condition: MeOH contained 0.2% Methanol Ammonia); OJ-H (4.6×250 mm, 5 μm); retention time: 4.34 minute.

### Example 182

### Synthesis of (R)—N-(3-(4-aminopiperidin-1-yl)-1,1-difluoro-3-oxo-1-(4-(1-(trifluoromethyl)cyclopropyl)phenyl)propan-2-yl)-6-(cyclopentyloxy)naphthalene-2-sulfonamide trifluoroacetate (Compound 182-1), and (S)—N-(3-(4-aminopiperidin-1-yl)-1,1-difluoro-3-oxo-1-(4-(1-(trifluoromethyl)cyclopropyl)phenyl)propan-2-yl)-6-(cyclopentyloxy) naphthalene-2-sulfonamide trifluoroacetate (Compound 182-2)

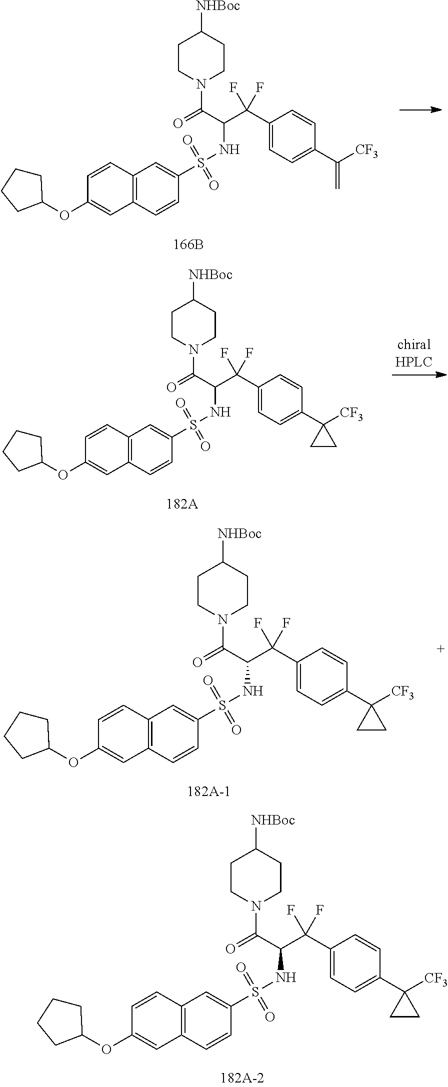 [SMILES: C=C(c1ccc(C(F)(F)C(NS(=O)(=O)c2ccc3cc(OC4CCCC4)ccc3c2)C(=O)N2CCC(NC(=O)OC(C)(C)C)CC2)cc1)C(F)(F)F ; CC(C)(C)OC(=O)NC1CCN(C(=O)C(NS(=O)(=O)c2ccc3cc(OC4CCCC4)ccc3c2)C(F)(F)c2ccc(C3(C(F)(F)F)CC3)cc2)CC1 ; CC(C)(C)OC(=O)NC1CCN(C(=O)[C@@H](NS(=O)(=O)c2ccc3cc(OC4CCCC4)ccc3c2)C(F)(F)c2ccc(C3(C(F)(F)F)CC3)cc2)CC1 ; CC(C)(C)OC(=O)NC1CCN(C(=O)[C@H](NS(=O)(=O)c2ccc3cc(OC4CCCC4)ccc3c2)C(F)(F)c2ccc(C3(C(F)(F)F)CC3)cc2)CC1]

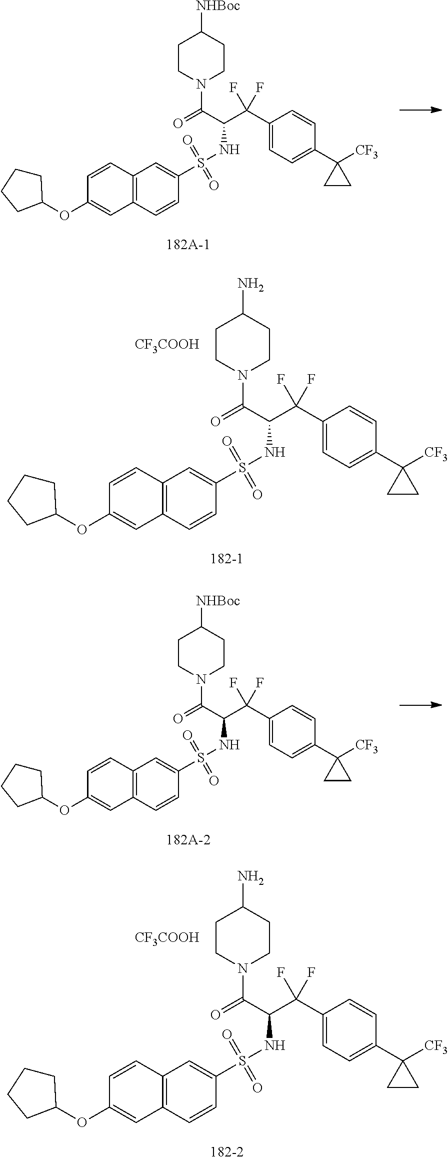 [SMILES: CC(C)(C)OC(=O)NC1CCN(C(=O)[C@@H](NS(=O)(=O)c2ccc3cc(OC4CCCC4)ccc3c2)C(F)(F)c2ccc(C3(C(F)(F)F)CC3)cc2)CC1 ; NC1CCN(C(=O)[C@@H](NS(=O)(=O)c2ccc3cc(OC4CCCC4)ccc3c2)C(F)(F)c2ccc(C3(C(F)(F)F)CC3)cc2)CC1 ; "CF3COOH" ; CC(C)(C)OC(=O)NC1CCN(C(=O)[C@H](NS(=O)(=O)c2ccc3cc(OC4CCCC4)ccc3c2)C(F)(F)c2ccc(C3(C(F)(F)F)CC3)cc2)CC1 ; NC1CCN(C(=O)[C@H](NS(=O)(=O)c2ccc3cc(OC4CCCC4)ccc3c2)C(F)(F)c2ccc(C3(C(F)(F)F)CC3)cc2)CC1 ; "CF3COOH"]

To a solution of potassium hydroxide (120.8 g) in water (150 mL) was added diethyl ether (150 ml) and then was cooled in an ice bath. To the cold solution was added N-nitroso-N-methylurea (50.07 g, 490 mmol) in several small portions. The reaction mixture was stirred at 0° C. for 10 minutes. The organic layer was separated and dried over potassium hydroxide to give the solution of diazomethylamine in diethyl ether, which was divided into five portions and added into a mixture of Compound 166B (3 g, 3.94 mmol) and Pd(OAc)₂ (300 mg) in THF (20 mL) five times. The mixture was stirred at 0° C. for 1 hour after each addition, at which time LCMS showed almost full consumption of starting material. The mixture was filtered through Celite and the filtrate was concentrated under reduced pressure. The residue was purified by preparative HPLC and chiral HPLC to furnish Compound 182A-1 and Compound 182A-2.

Compound 182A-1: LC-MS (ESI) m/z: 788 [M+Na]⁺. Chiral separation condition: n-hexane/EtOH contained 0.1% DEA, 80/20; OZ-H (4.6×250 mm, 5 μm); retention time: 4.66 minute.

Compound 182A-2: LC-MS (ESI) m/z: 788 [M+Na]⁺. Chiral separation condition: n-hexane/EtOH contained 0.1% DEA, 80/20; OZ-H (4.6×250 mm, 5 μm); retention time: 6.16 minute.

Compound 182-1 was synthesized by employing the procedure described for Compound 6 using Compound 182A-1 in lieu of Compound 6C: LC-MS (ESI) m/z: 666 [M+H]⁺; ¹H-NMR (CD₃OD, 400 MHz): δ (ppm) 0.88-2.06 (m, 17H), 2.61-3.16 (m, 2H), 4.01-4.58 (m, 2H), 4.96-5.06 (m, 2H), 7.22-7.62 (m, 7H), 7.74-7.91 (m, 2H), 8.20-8.21 (m, 1H).

Compound 182-2 was synthesized by employing the procedure described for Compound 6 using Compound 182A-2 in lieu of Compound 6C: LC-MS (ESI) m/z: 666 [M+H]⁺; ¹H-NMR (CD₃OD, 400 MHz): δ (ppm) 0.88-2.06 (m, 17H), 2.63-3.26 (m, 2H), 4.01-4.46 (m, 2H), 4.93-5.10 (m, 2H), 7.22-7.62 (m, 7H), 7.74-7.91 (m, 2H), 8.20-8.22 (m, 1H).

### Example 183

### Synthesis of N—((R)-3-((1R,5S,8R)-8-amino-3-azabicyclo[3.2.1]octan-3-yl)-1-(4′-chloro-[1,1′-biphenyl]-4-yl)-1,1-difluoro-3-oxopropan-2-yl)-4-(cyclohexylmethoxy) benzenesulfonamide (Compound 183-1), and N—((S)-3-((1R,5S,8S)-8-amino-3-azabicyclo[3.2.1]octan-3-yl)-1-(4′-chloro-[1,1′-biphenyl]-4-yl)-1,1-difluoro-3-oxopropan-2-yl)-4-(cyclohexylmethoxy)benzenesulfonamide (Compound 183-2)

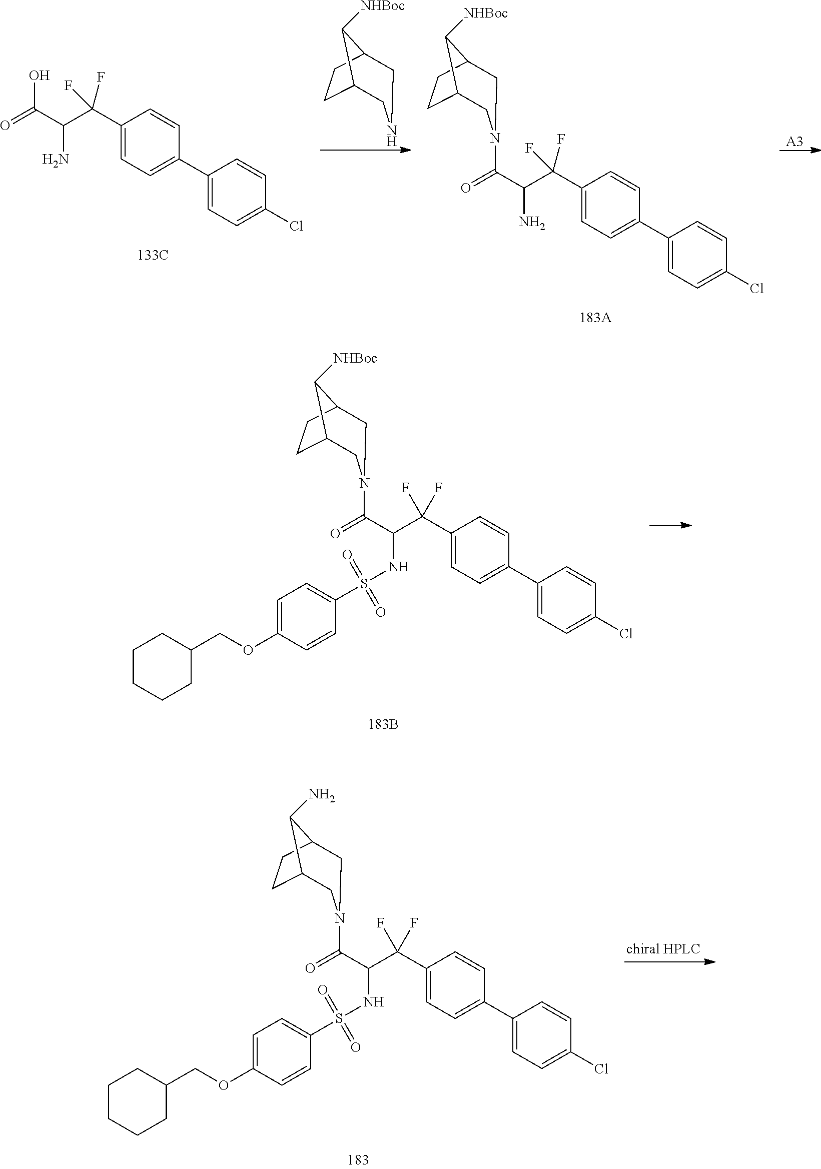

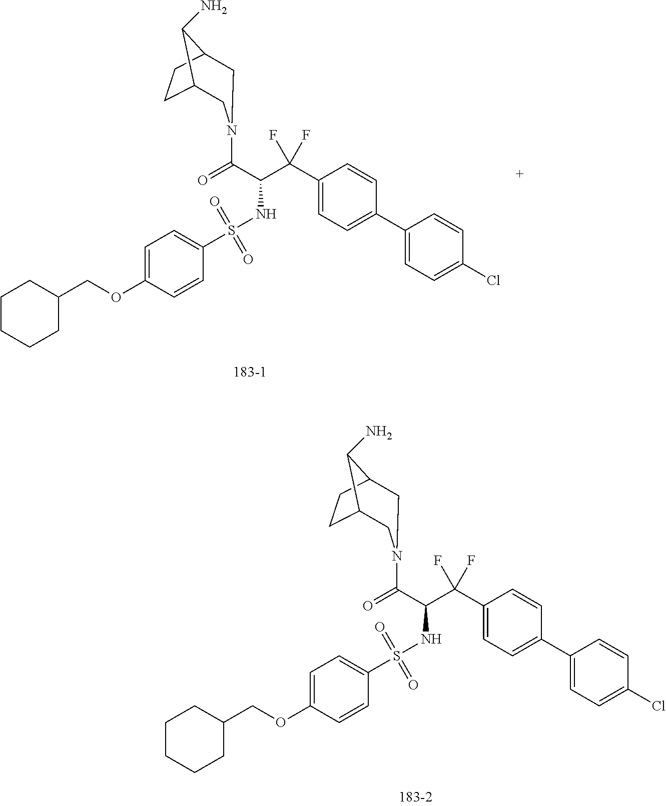 [SMILES: NC1C2CCC1CN(C(=O)[C@@H](NS(=O)(=O)c1ccc(OCC3CCCCC3)cc1)C(F)(F)c1ccc(-c3ccc(Cl)cc3)cc1)C2 ; NC1C2CCC1CN(C(=O)[C@H](NS(=O)(=O)c1ccc(OCC3CCCCC3)cc1)C(F)(F)c1ccc(-c3ccc(Cl)cc3)cc1)C2]

Compounds 183A, 183B, and 183 were synthesized by employing the procedures described for Compound B4, Compounds 2D, and 1 using tert-butyl (1R,5S,8r)-3-azabicyclo[3.2.1]octan-8-ylcarbamate, Compounds 133C, 183A, Compound A3, and Compound 183B in lieu of Compounds B4-1, B2-6, B2, Compounds 2C, and 1A.

Compound 183A: LC-MS (ESI) m/z: 520 [M+H]⁺.

Compound 183B: LC-MS (ESI) m/z: 794 [M+Na]⁺.

Compound 183 was separated with chiral HPLC to afford Compound 183-1 and Compound 183-2.

Compound 183-1: LC-MS (ESI) m/z: 672 [M+H]⁺; ¹H-NMR (CD₃OD, 400 MHz): δ (ppm) 0.88-1.18 (m, 7H), 1.75-2.20 (m, 12H), 2.84-3.31 (m, 2H), 3.32-4.01 (m, 4H), 6.80-7.68 (m, 12H). Chiral separation condition: MeOH contained 0.5% NH₄OH; IC (150×4.6 mm, 5 μm); retention time: 7.03 minute.

Compound 183-2: LC-MS (ESI) m/z: 672 [M+H]⁺. ¹H-NMR (CD₃OD, 400 MHz): δ (ppm) 0.79-1.21 (m, 5H), 1.25-1.77 (m, 12H), 2.26-2.33 (m, 2H), 2.93-3.01 (m, 1H), 3.21-4.19 (m, 5H), 6.60-7.65 (m, 12H). Chiral separation condition: MeOH contained 0.5% NH₄OH; IC (150×4.6 mm, 5 μm); retention time: 5.37 minute.

### Example 184

### Synthesis of N—((S)-3-((1R,5S,8S)-8-amino-3-azabicyclo[3.2.1]octan-3-yl)-1-(4′-chloro-[1,1′-biphenyl]-4-yl)-1,1-difluoro-3-oxopropan-2-yl)-6-(cyclopentyloxy) naphthalene-2-sulfonamide (Compound 184-2), and N—((R)-3-((1R,5S,8R)-8-amino-3-azabicyclo[3.2.1]octan-3-yl)-1-(4′-chloro-[1,1′-biphenyl]-4-yl)-1,1-difluoro-3-oxopropan-2-yl)-6-(cyclopentyloxy)naphthalene-2-sulfonamide (Compound 184-1)

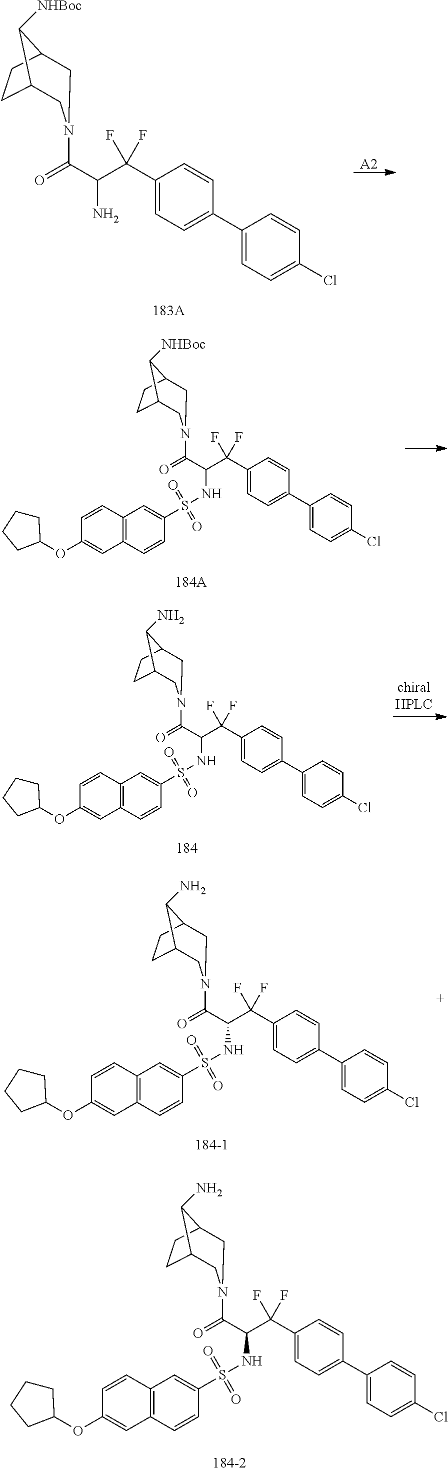 [SMILES: CC(C)(C)OC(=O)NC1C2CCC1CN(C(=O)C(N)C(F)(F)c1ccc(-c3ccc(Cl)cc3)cc1)C2 ; "A2" ; CC(C)(C)OC(=O)NC1C2CCC1CN(C(=O)C(NS(=O)(=O)c1ccc3cc(OC4CCCC4)ccc3c1)C(F)(F)c1ccc(-c3ccc(Cl)cc3)cc1)C2 ; NC1C2CCC1CN(C(=O)C(NS(=O)(=O)c1ccc3cc(OC4CCCC4)ccc3c1)C(F)(F)c1ccc(-c3ccc(Cl)cc3)cc1)C2 ; NC1C2CCC1CN(C(=O)[C@@H](NS(=O)(=O)c1ccc3cc(OC4CCCC4)ccc3c1)C(F)(F)c1ccc(-c3ccc(Cl)cc3)cc1)C2 ; NC1C2CCC1CN(C(=O)[C@H](NS(=O)(=O)c1ccc3cc(OC4CCCC4)ccc3c1)C(F)(F)c1ccc(-c3ccc(Cl)cc3)cc1)C2]

Compounds 184A and 184 were synthesized by employing the procedures described for Compounds 2D and 1 using Compound 183A, Compound A2, and Compound 184A in lieu of Compounds B2, 2C, and 1A.

Compound 184A: LC-MS (ESI) m/z: 816 [M+Na]⁺.

Compound 184 was separated with chiral HPLC to afford Compound 184-1 and Compound 184-2.

Compound 184-1: LC-MS (ESI) m/z: 694 [M+H]⁺; ¹H-NMR (CD₃OD, 400 MHz): δ (ppm) 1.31-2.50 (m, 14H), 3.02-3.17 (m, 1H), 3.32-3.69 (m, 2H), 3.97-4.22 (m, 2H), 4.68-4.76 (m, 1H), 5.01-5.34 (m, 1H), 6.89-8.09 (m, 14H). Chiral separation condition: MeOH contained 0.5% NH₄OH; (R, R)-WHELK-O1 (250×4.6 mm, 5 μm); retention time: 6.74 minute.

Compound 184-2: LC-MS (ESI) m/z: 694 [M+H]⁺; ¹H-NMR (CD₃OD, 400 MHz): δ (ppm) 0.75-1.23 (m, 2H), 1.68-2.44 (m, 12H), 2.85-3.17 (m, 2H), 3.32-3.92 (m, 3H), 4.78-5.06 (m, 2H), 7.02-7.15 (m, 2H), 7.39-7.85 (m, 11H), 8.13-8.18 (m, 1H). Chiral separation condition: MeOH contained 0.5% NH₄OH; (R, R)-WHELK-O1 (250×4.6 mm, 5 μm); retention time: 9.11 minute.

### Example 185

### Synthesis of (R)—N-(3-(4-aminopiperidin-1-yl)-1-(4′-chloro-[1,1′-biphenyl]-4-yl)-1,1-difluoro-3-oxopropan-2-yl)-4-(2-(cyclopentyloxy)ethyl)benzenesulfonamide trifluoroacetate (Compound 185-1), and (S)—N-(3-(4-aminopiperidin-1-yl)-1-(4′-chloro-[1,1′-biphenyl]-4-yl)-1,1-difluoro-3-oxopropan-2-yl)-4-(2-(cyclopentyloxy) ethyl) benzenesulfonamide trifluoroacetate (Compound 185-2)

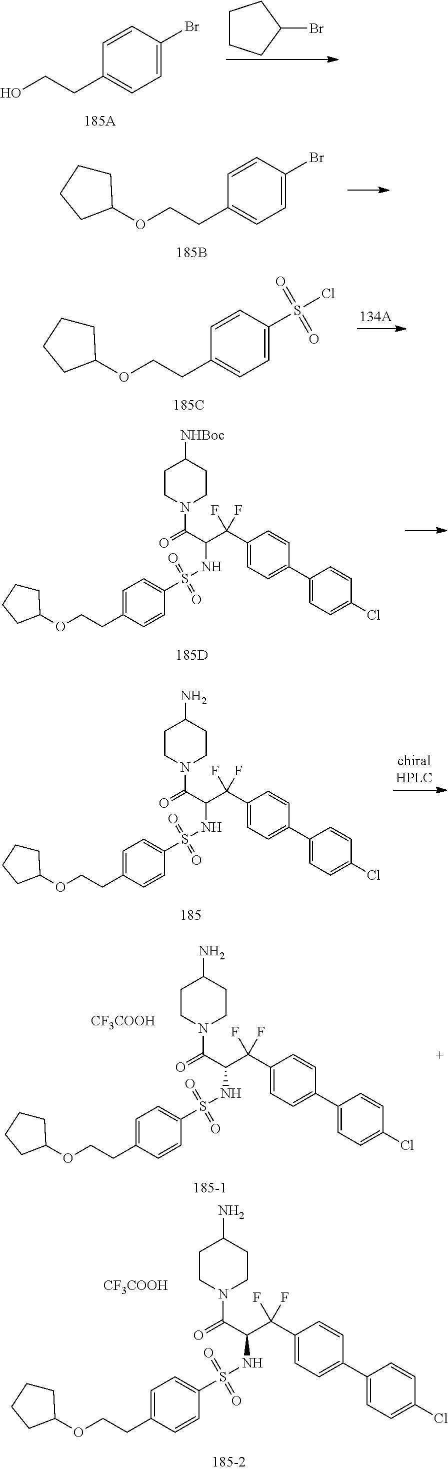

Compounds 185B, 185C, 185D, and 185 were synthesized by employing the procedures described for Compound A5-2, Compounds 177B, 2D, and 1 using Compounds 185A at 50° C., 185B, 185C, 134A, and 185D in lieu of Compound A5-1 at 30° C., Compounds 177A, 2C, Compound B2, and Compound 1A.

Compound 185B: ¹H-NMR (CDCl₃, 400 MHz): δ (ppm) 1.49-1.70 (m, 8H), 2.80 (t, J=7.2 Hz, 2H), 3.55 (t, J=7.2 Hz, 2H), 3.85-3.88 (m, 1H), 7.09 (d, J=8.4 Hz, 2H), 7.39 (d, J=8.0 Hz, 2H).

Compound 185C: ¹H-NMR (CDCl₃, 400 MHz): δ (ppm) 1.42-1.64 (m, 8H), 2.89 (t, J=6.4 Hz, 2H), 3.56 (t, J=6.4 Hz, 2H), 3.78-3.83 (m, 1H), 7.40 (d, J=8.4 Hz, 2H), 7.87 (d, J=8.4 Hz, 2H).

Compound 185D: LC-MS (ESI) m/z: 746[M+H]⁺.

Compound 185 was separated with chiral HPLC to afford Compound 185-1 and Compound 185-2.

Compound 185-1: LC-MS (ESI) m/z: 646 [M+H]⁺. ¹H-NMR (CD₃OD, 400 MHz): δ (ppm) 1.53-1.67 (m, 10H), 2.02-2.22 (m, 3H), 2.69-2.81 (m, 3H), 3.14 (s, 1H), 3.48-3.65 (m, 2H), 4.20-4.42 (m, 2H), 5.03-5.09 (m, 1H), 5.34-5.36 (m, 1H), 7.20-7.29 (m, 2H), 7.45-7.59 (m, 7H), 7.64-7.69 (m, 3H). Chiral separation condition: n-Hexane/EtOH contained 0.1% DEA, 50/50; OZ-H (250×4.6 mm, 5 μm); retention time: 8.44 minute.

Compound 185-2: LC-MS (ESI) m/z: 664 [M+H]⁺. ¹H-NMR (CD₃OD, 400 MHz): δ (ppm) 1.52-1.73 (m, 10H), 1.99-2.22 (m, 3H), 2.44-2.96 (m, 3H), 3.10-3.14 (m, 1H), 3.46-3.56 (m, 2H), 3.83-4.41 (m, 2H), 4.96-5.10 (m, 1H), 5.35 (s, 1H), 7.20-7.29 (m, 2H), 7.45-7.58 (m, 7H), 7.64-7.69 (m, 3H). Chiral separation condition: n-Hexane/EtOH contained 0.1% DEA, 50/50; OZ-H (250×4.6 mm, 5 μm); retention time: 7.41 minute.

### Example 186

### Synthesis of N—((R)-3-((S)-3-aminopyrrolidin-1-yl)-1-(4-chlorophenyl)-1,1-difluoro-3-oxopropan-2-yl)-4-(cyclohexylmethoxy)benzenesulfonamide trifluoroacetate (Compound 186-1), and N—((S)-3-((S)-3-aminopyrrolidin-1-yl)-1-(4-chlorophenyl)-1,1-difluoro-3-oxopropan-2-yl)-4-(cyclohexylmethoxy)benzenesulfonamide trifluoroacetate (Compound 186-2)

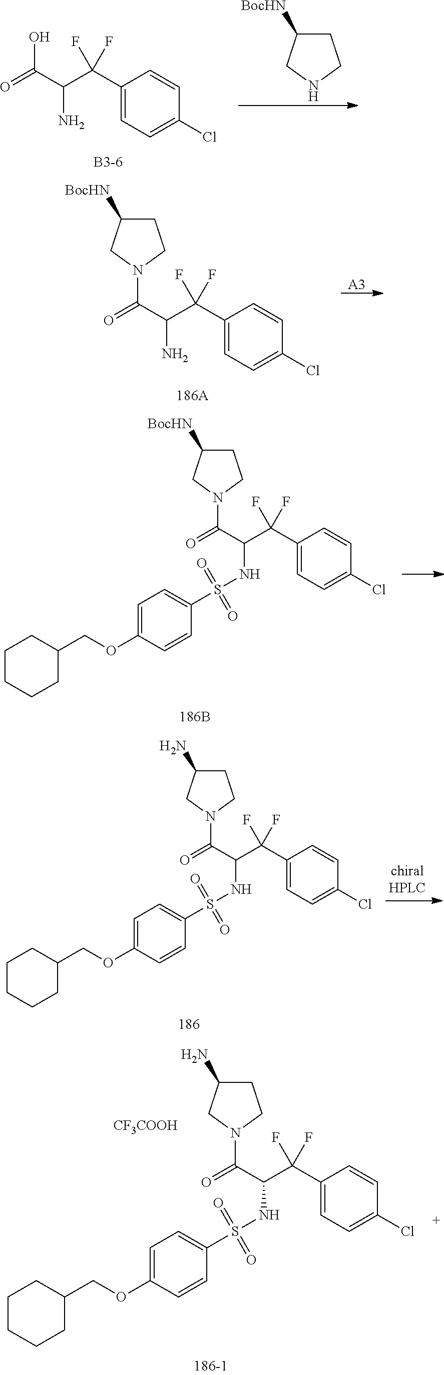

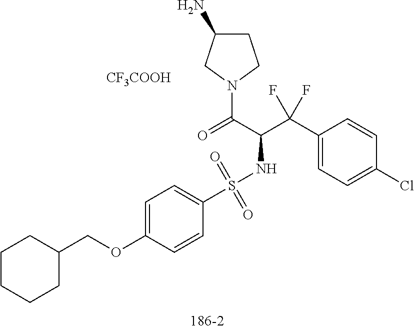 [SMILES: N[C@H]1CCN(C(=O)[C@H](NS(=O)(=O)c2ccc(OCC3CCCCC3)cc2)C(F)(F)c2ccc(Cl)cc2)C1 ; "CF3COOH"]

Compounds 186A, 186B, and 186 were synthesized by employing the procedures described for Compound B4, Compounds 2D, and 1 using (S)-tert-butyl pyrrolidin-3-ylcarbamate, Compounds B3-6, A3, Compounds 186A, and 186B in lieu of Compounds B4-1, B2-6, Compound 2C, Compound B2, and Compound 1A.

Compound 186A: LC-MS (ESI) m/z: 404 [M+H]⁺; ¹H-NMR (DMSO-d₆, 400 MHz): δ (ppm) 1.37-1.40 (m, 9H), 1.69-1.94 (m, 2H), 2.86-3.17 (m, 2H), 3.41-4.14 (m, 4H), 6.89-7.53 (m, 4H).

Compound 186B: LC-MS (ESI) m/z: 678 [M+Na]⁺; ¹H-NMR (CDCl₃, 400 MHz): δ (ppm) 1.05-1.27 (m, 6H), 1.41-1.58 (m, 9H), 1.71-1.88 (m, 7H), 3.02-3.70 (m, 4H), 3.79-3.82 (m, 2H), 4.06-4.51 (m, 2H), 6.87-6.99 (m, 2H), 7.29-7.35 (m, 4H), 7.56-7.75 (m, 2H).

Compound 186 was separated with chiral HPLC to afford Compound 186-1 and Compound 186-2.

Compound 186-1: LC-MS (ESI) m/z: 556 [M+H]⁺; ¹H-NMR (CD₃OD, 400 MHz): δ (ppm) 1.09-1.40 (m, 5H), 1.73-2.46 (m, 8H), 3.45-3.96 (m, 7H), 4.52-4.73 (m, 1H), 6.88-6.92 (m, 2H), 7.29-7.35 (m, 2H), 7.39-7.45 (m, 2H), 7.50 (d, J=9.2 Hz, 1H), 7.59 (d, J=8.8 Hz, 1H). Chiral separation condition: n-Hexane/EtOH contained 0.1% DEA, 60/40; S, S-WHELK-O1 (4.6×250 mm, 5 μm); retention time: 8.86 minute.

Compound 186-2: LC-MS (ESI) m/z: 556 [M+H]⁺; ¹H-NMR (CD₃OD, 400 MHz): δ (ppm) 1.09-1.40 (m, 5H), 1.72-2.45 (m, 8H), 3.32-3.94 (m, 7H), 4.58-4.64 (m, 1H), 6.90-6.94 (m, 2H), 77.34-7.44 (m, 4H), 7.52-7.58 (m, 2H). Chiral separation condition: n-Hexane/EtOH contained 0.1% DEA, 60/40; S, S-WHELK-O1 (4.6×250 mm, 5 μm); retention time: 7.26 minute.

### Example 187

### Synthesis of (R)—N-(3-(4-aminopiperidin-1-yl)-1-(4-bromophenyl)-1,1-difluoro-3-oxopropan-2-yl)-6-(cyclopentyloxy)-N-(2,2,2-trifluoroethyl)naphthalene-2-sulfonamide trifluoroacetate (Compound 187-1), and (S)—N-(3-(4-aminopiperidin-1-yl)-1-(4-bromophenyl)-1,1-difluoro-3-oxopropan-2-yl)-6-(cyclopentyloxy)-N-(2,2,2-trifluoroethyl)naphthalene-2-sulfonamide trifluoroacetate (Compound 187-2)

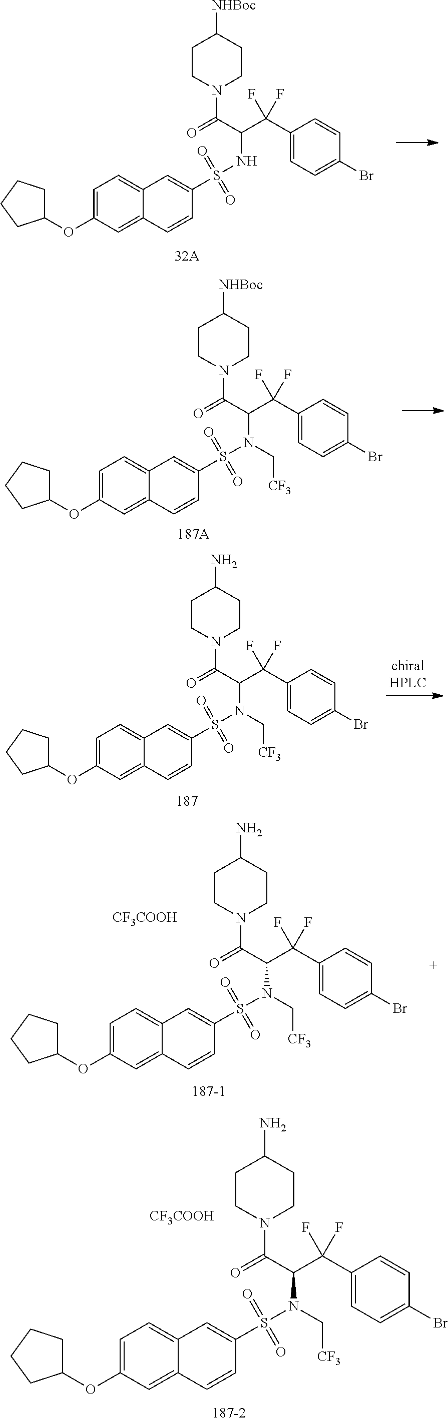

A mixture of Compound 32A (500 mg, 0.7 mmol), K₂CO₃ (290 mg, 2.1 mmol), N,N-diisopropylethylamine (900 mg, 7 mmol), and 2,2,2-trifluoroethyl trifluoromethanesulfonate (1.6 g, 7 mmol) in DMSO (15 mL) was stirred at 80° C. overnight. The reaction mixture was diluted with water (30 mL) and extracted with ethyl acetate (50 mL×2). The combined organic extracts were washed with water (20 mL×3) and brine (50 mL), dried over anhydrous sodium sulfate, filtered, and concentrated under reduced pressure. The residue was purified by silica gel column chromatography to afford Compound 187A. LC-MS (ESI) m/z: 818 [M+H]⁺.

Compound 186 was synthesized by employing the procedure described for Compound 6 using Compound 187A in lieu of Compound 6C, which was separated with chiral HPLC to furnish Compound 187-1 and Compound 187-2.

Compound 187-1: LC-MS (ESI) m/z: 718 [M+H]⁺; ¹H-NMR (CD₃OD, 400 MHz): δ (ppm) 1.62-1.97 (m, 12H), 2.46-2.74 (m, 1H), 2.96-3.08 (m, 1H), 3.94-4.01 (m, 1H), 4.15-4.24 (m, 2H), 5.23-5.25 (m, 2H), 5.38-5.47 (m, 2H), 7.12-7.36 (m, 6H), 7.53-7.59 (m, 1H), 7.75-7.88 (m, 2H), 8.24 (s, 1H). Chiral separation condition: MeOH contained 0.5% NH₄OH; IC (4.6×150 mm, 5 μm); retention time: 4.78 minute.

Compound 187-2: LC-MS (ESI) m/z: 718 [M+H]⁺; ¹H-NMR (CD₃OD, 400 MHz): δ (ppm) 1.62-2.15 (m, 12H), 2.42-2.75 (m, 1H), 2.99-3.14 (m, 1H), 3.95-4.02 (m, 1H), 4.19-4.46 (m, 2H), 4.84-4.95 (m, 3H), 5.38-5.46 (m, 1H), 7.12-7.36 (m, 6H), 7.51-7.60 (m, 1H), 7.76-7.88 (m, 2H), 8.24 (s, 1H). Chiral separation condition: MeOH contained 0.5% NH₄OH; IC (4.6×150 mm, 5 μm); retention time: 5.48 minute.

### Example 188

### Synthesis of (R)—N-(1-(4′-chloro-[1,1′-biphenyl]-4-yl)-1,1-difluoro-3-oxo-3-(4,7-diazaspiro[2.5]octan-7-yl)propan-2-yl)-6-(cyclopentyloxy)naphthalene-2-sulfonamide trifluoroacetate (Compound 188-1), and (S)—N-(1-(4′-chloro-[1,1′-biphenyl]-4-yl)-1,1-difluoro-3-oxo-3-(4,7-diazaspiro[2.5]octan-7-yl)propan-2-yl)-6-(cyclopentyloxy) naphthalene-2-sulfonamide trifluoroacetate (Compound 188-2)

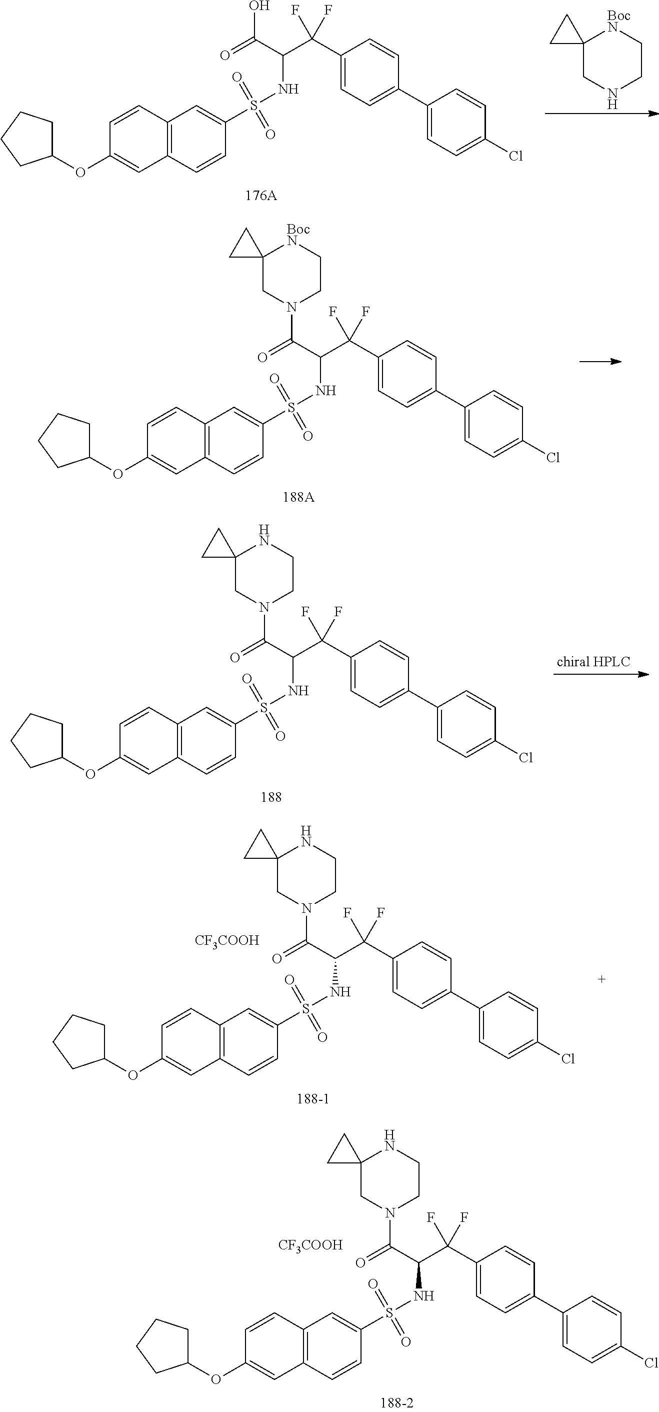

Compounds 188A and 188 were synthesized by employing the procedures described for Compound B4 and Compound 6 using tert-butyl 4,7-diazaspiro[2.5]octane-4-carboxylate, Compound 176A, and Compound 189A in lieu of Compounds B4-1, B2-6, and Compound 6C.

Compound 188A: LC-MS (ESI) m/z: 780[M+H]⁺.

Compound 188 was separated with chiral-HPLC to afford Compound 188-1 and Compound 188-2.

Compound 188-1: LC-MS (ESI) m/z: 680[M+H]⁺. ¹H NMR (MeOD, 400 MHz): δ (ppm) 0.54-1.14 (m, 4H), 1.71-1.84 (m, 6H), 2.03 (s, 2H), 3.00-3.13 (m, 2H), 3.40-3.49 (m, 2H), 3.71-3.78 (m, 1H), 3.94-4.20 (m, 1H), 4.75-4.79 (m, 1H), 4.99-5.10 (m, 1H), 7.02-7.17 (m, 2H), 7.37-7.59 (m, 8H), 7.72-7.85 (m, 3H), 8.07-8.19 (m, 1H). Chiral separation condition: MeOH contained 0.2% Methanol Ammonia; (R,R)-Whelk-O1 (250×4.6 mm, 5 μm); retention time: 8 minute.

Compound 188-2: LC-MS (ESI) m/z: 680[M+H]⁺. ¹H NMR (MeOD, 400 MHz): δ (ppm) 0.56-1.18 (m, 4H), 1.71-1.84 (m, 6H), 2.03 (s, 2H), 3.07-3.13 (m, 2H), 3.45-3.50 (m, 2H), 3.77 (s, 1H), 3.94-4.20 (m, 1H), 4.80 (s, 1H), 4.99-5.09 (m, 1H), 7.01-7.17 (m, 2H), 7.34-7.58 (m, 8H), 7.71-7.84 (m, 3H), 8.07-8.19 (m, 1H). Chiral separation condition: MeOH contained 0.2% Methanol Ammonia; (R,R)-Whelk-O1 (250×4.6 mm, 5 μm); retention time: 10.23 minute.

### Example 189

### Synthesis of N—((R)-3-((R)-3-aminopyrrolidin-1-yl)-1-(4-chlorophenyl)-1,1-difluoro-3-oxopropan-2-yl)-4-(cyclohexylmethoxy)benzenesulfonamide trifluoroacetate (Compound 189-1), and N—((S)-3-((R)-3-aminopyrrolidin-1-yl)-1-(4-chlorophenyl)-1,1-difluoro-3-oxopropan-2-yl)-4-(cyclohexylmethoxy)benzenesulfonamide trifluoroacetate (Compound 189-2)

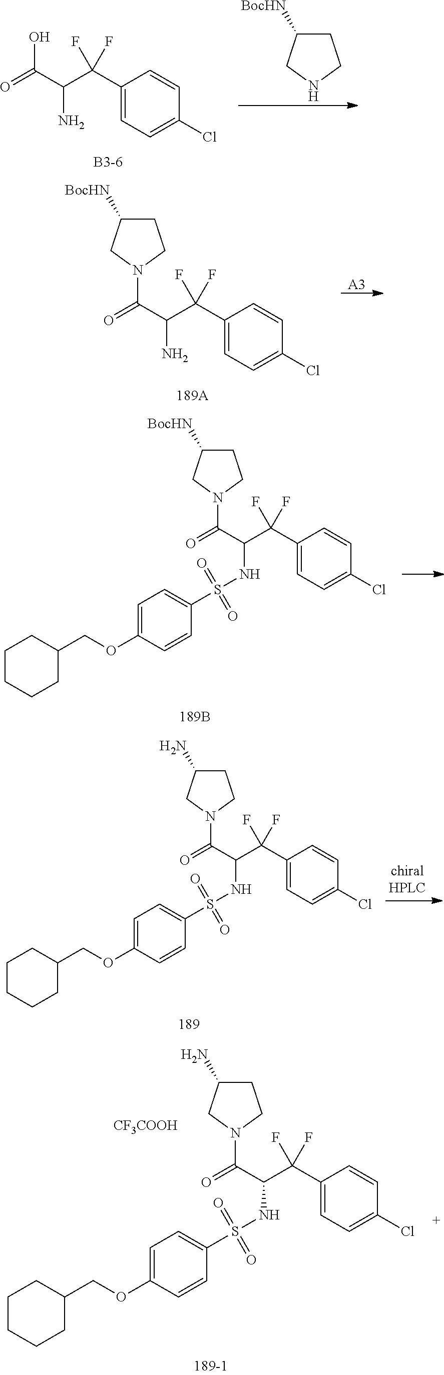

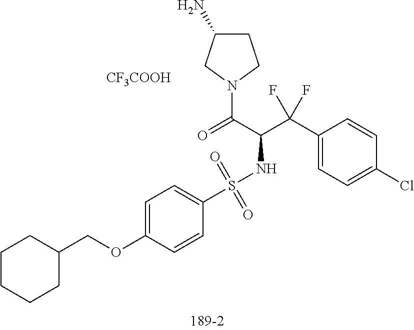 [SMILES: N[C@@H]1CCN(C(=O)[C@H](NS(=O)(=O)c2ccc(OCC3CCCCC3)cc2)C(F)(F)c2ccc(Cl)cc2)C1 ; "CF3COOH"]

Compounds 189A, 189B, and 189 were synthesized by employing the procedures described for Compound B4, Compounds 2D, and 1 using (R)-tert-butyl pyrrolidin-3-ylcarbamate, Compounds B3-6, A3, Compounds 189A, and 189B in lieu of Compounds B4-1, B2-6, Compound 2C, Compound B2, and Compound 1A.

Compound 189A: LC-MS (ESI) m/z: 404 [M+H]⁺; ¹H-NMR (DMSO-d₆, 400 MHz): δ (ppm) 1.37-1.40 (m, 9H), 1.69-1.94 (m, 2H), 2.78-3.33 (m, 2H), 3.43-4.10 (m, 4H), 6.89-7.54 (m, 4H).

Compound 189B: LC-MS (ESI) m/z: 678 [M+Na]⁺.

Compound 189 was separated with chiral HPLC to afford Compound 189-1 and Compound 189-2.

Compound 189-1: LC-MS (ESI) m/z: 556 [M+H]⁺; ¹H-NMR (CD₃OD, 400 MHz): δ (ppm) 1.12-1.37 (m, 5H), 1.79-2.43 (m, 8H), 3.37-3.94 (m, 7H), 4.58-4.64 (m, 1H), 6.90-6.94 (m, 2H), 77.34-7.44 (m, 4H), 7.52-7.57 (m, 2H). Chiral separation condition: MeOH contained 0.5% NH₄OH; OZ-H (4.6×250 mm, 5 μm); retention time: 5.36 minute.

Compound 189-2: LC-MS (ESI) m/z: 556 [M+H]⁺; ¹H-NMR (CD₃OD, 400 MHz): δ (ppm) 1.09-1.40 (m, 5H), 1.73-2.47 (m, 8H), 3.45-3.97 (m, 7H), 4.52-4.73 (m, 1H), 6.88-6.96 (m, 2H), 7.29-7.35 (m, 2H), 7.41-7.51 (m, 2H), 7.50 (d, J=8.8 Hz, 1H), 7.59 (d, J=8.8 Hz, 1H). Chiral separation condition: MeOH contained 0.5% NH₄OH; OZ-H (4.6×250 mm, 5 μm); retention time: 3.73 minute.

### Example 190

### Synthesis of N—((R)-1-(4′-chloro-2-methoxy-[1,1′-biphenyl]-4-yl)-1,1-difluoro-3-((3aR,6aS)-hexahydropyrrolo[3,4-c]pyrrol-2(1H)-yl)-3-oxopropan-2-yl)-4-(cyclohexylmethoxy)benzenesulfonamide trifluoroacetate (Compound 190-1), and N—((S)-1-(4′-chloro-2-methoxy-[1,1′-biphenyl]-4-yl)-1,1-difluoro-3-((3aR,6aS)-hexahydropyrrolo [3,4-c]pyrrol-2(1H)-yl)-3-oxopropan-2-yl)-4-(cyclohexylmethoxy) benzenesulfonamide trifluoroacetate (Compound 190-2)

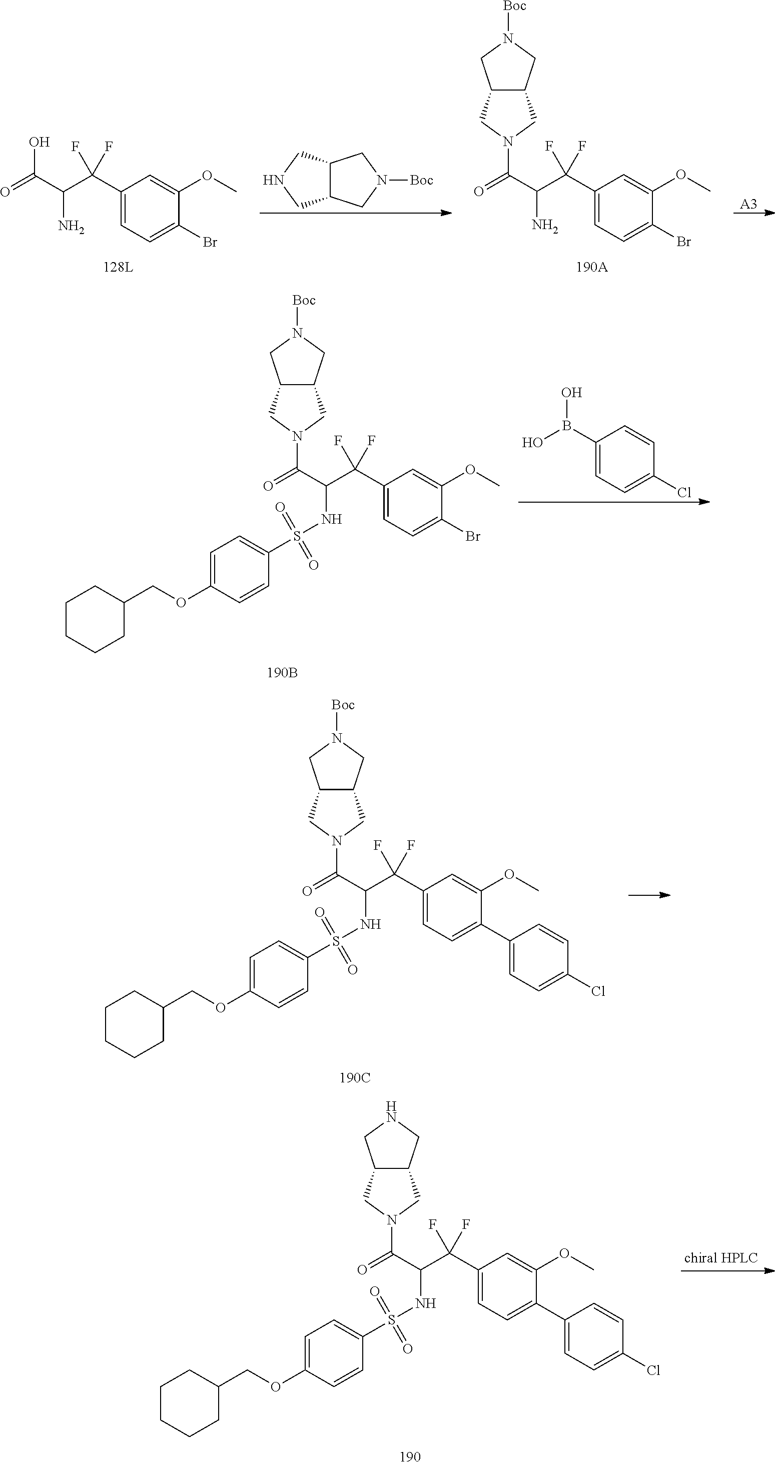

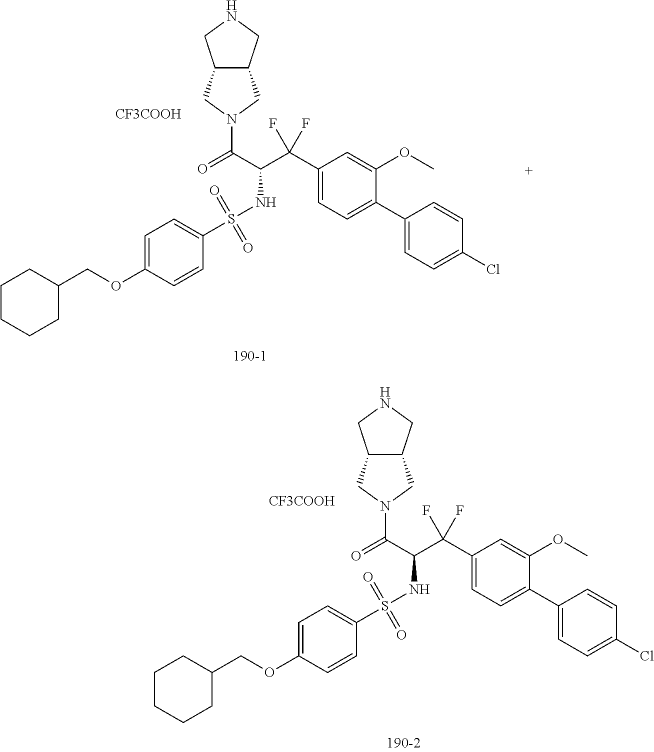 [SMILES: COc1cc(C(F)(F)[C@H](NS(=O)(=O)c2ccc(OCC3CCCCC3)cc2)C(=O)N2C[C@H]3CNC[C@H]3C2)ccc1-c1ccc(Cl)cc1 ; "CF3COOH" ; COc1cc(C(F)(F)[C@@H](NS(=O)(=O)c2ccc(OCC3CCCCC3)cc2)C(=O)N2C[C@H]3CNC[C@H]3C2)ccc1-c1ccc(Cl)cc1 ; "CF3COOH"]

Compounds 190A, 190B, 190C, and 190 were synthesized by employing the procedures described for Compound B4, Compounds 2D, 14C, and 1 using (3aR,6aS)-tert-butyl hexahydropyrrolo[3,4-c]pyrrole-2(1H)-carboxylate, Compounds 128L, 190A, Compound A3, 4-chlorophenylboronic acid, Compounds 190B, and 190C in lieu of Compounds B4-1, B2-6, B2, Compounds 2C, 14B, 14A, and 1A.

Compound 190A: LC-MS (ESI) m/z: 504 [M+H]⁺.

Compound 190B: LC-MS (ESI) m/z: 700 [M−55]⁺.

Compound 190C: LC-MS (ESI) m/z: 732 [M−55]⁺.

Compound 190 was separated with chiral HPLC to give Compound 190-1 and Compound 190-2.

Compound 190-1: LC-MS (ESI) m/z: 688 [M+H]⁺; ¹H-NMR (DMSO-d₆, 400 MHz): δ (ppm) 0.90-0.93 (m, 2H), 1.18-1.21 (m, 3H), 1.63-1.70 (m, 6H), 2.50-3.20 (m, 5H), 3.34-3.80 (m, 10H), 4.60-4.71 (m, 1H), 6.80-6.87 (m, 2H), 6.97-7.10 (m, 2H), 7.26-7.32 (m, 1H), 7.40-7.56 (m, 6H), 8.58-8.70 (m, 1H), 8.88-8.96 (m, 2H). Chiral separation condition: n-Hexane/EtOH contained 0.1% DEA, 40/60; OZ-H (4.6×250 mm, 5 μm); retention time: 16.19 minute.

Compound 190-2: LC-MS (ESI) m/z: 688 [M+H]⁺; ¹H-NMR (DMSO-d₆, 400 MHz): δ (ppm) 0.90-0.93 (m, 2H), 1.18-1.21 (m, 3H), 1.63-1.70 (m, 6H), 2.66-3.07 (m, 5H), 3.21-3.80 (m, 10H), 4.63-4.69 (m, 1H), 6.80-6.87 (m, 2H), 6.97-7.10 (m, 2H), 7.26-7.32 (m, 1H), 7.40-7.56 (m, 6H), 8.58-8.70 (m, 1H), 8.83-8.96 (m, 2H). Chiral separation condition: n-Hexane/EtOH contained 0.1% DEA, 40/60; OZ-H (4.6×250 mm, 5 μm); retention time: 9.13 minute.

### Example 191

### Synthesis of (R)—N-(1-(4′-chloro-[1,1′-biphenyl]-4-yl)-1,1-difluoro-3-oxo-3-(1,6-diazaspiro[3.3]heptan-1-yl)propan-2-yl)-6-(cyclopentyloxy)naphthalene-2-sulfonamide trifluoroacetate (Compound 191-1), and (S)—N-(1-(4′-chloro-[1,1′-biphenyl]-4-yl)-1,1-difluoro-3-oxo-3-(1,6-diazaspiro[3.3]heptan-1-yl)propan-2-yl)-6-(cyclopentyloxy) naphthalene-2-sulfonamide trifluoroacetate (Compound 191-2)

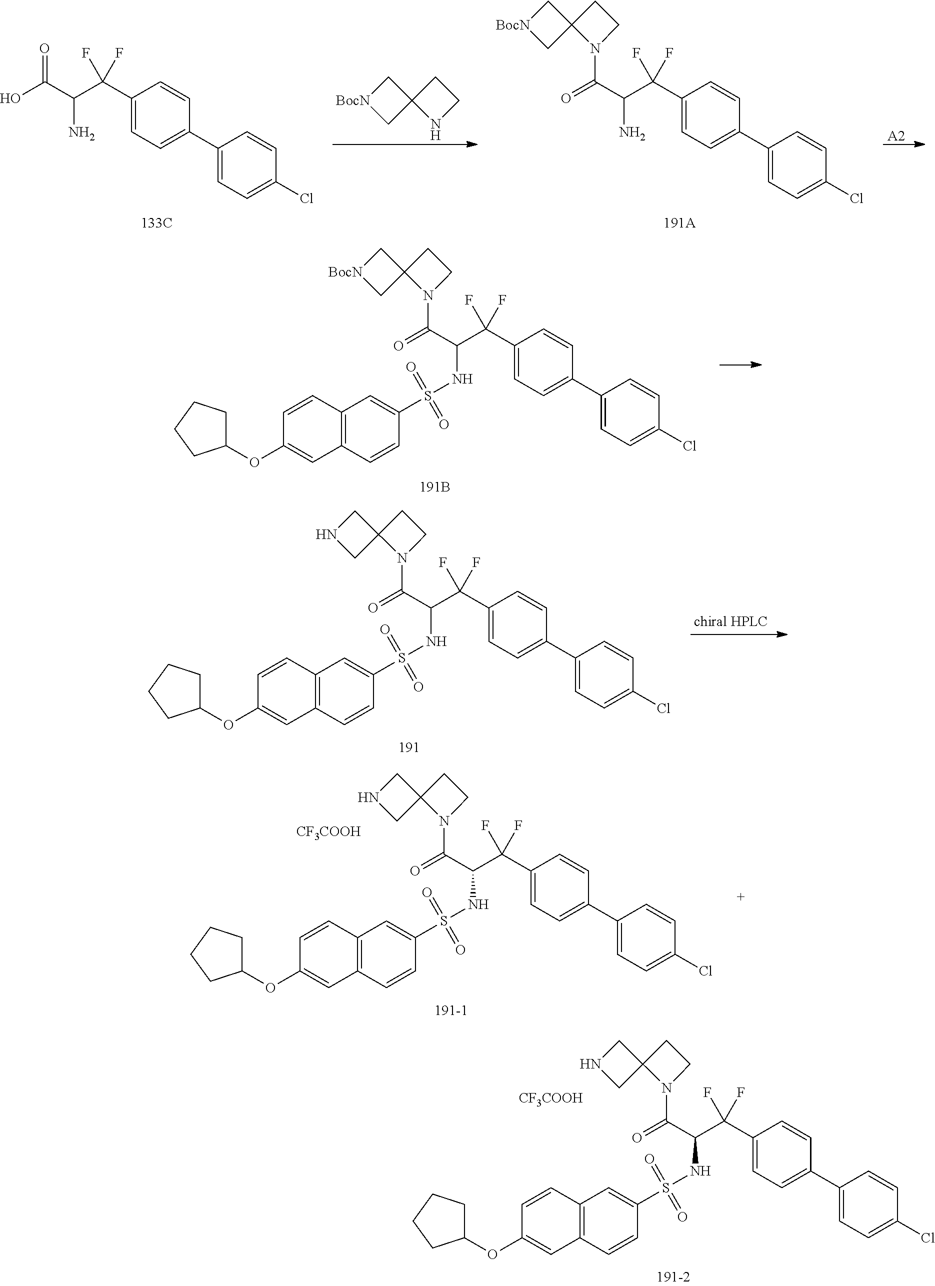

Compounds 191A, 191B, and 191 were synthesized by employing the procedures described for Compound B4, Compounds 2D, and 1 using tert-butyl 1,6-diazaspiro[3.3]heptane-6-carboxylate, Compounds 133C, 191A, Compound A2, and Compound 191B in lieu of Compounds B4-1, B2-6, B2, Compounds 2C, and 1A.

Compound 191A: LC-MS (ESI) m/z: 492 [M+H]⁺.

Compound 191B was used directly in next step without further purification. LC-MS (ESI) m/z: 766 [M+H]⁺.

Compound 191 was separated with chiral HPLC to give Compound 191-1 and Compound 191-2.

Compound 191-1: LC-MS (ESI) m/z: 666 [M+H]⁺; ¹H-NMR (CD₃OD, 400 MHz): δ (ppm) 1.70-1.74 (m, 2H), 1.85-1.87 (m, 4H), 2.01-2.07 (m, 2H), 2.21-2.28 (m, 1H), 2.40-2.46 (m, 1H), 3.60-3.65 (m, 1H), 3.90-3.99 (m, 2H), 4.15-4.18 (m, 2H), 4.37-4.41 (m, 2H), 4.85-4.86 (m, 1H), 7.12 (s, 1H), 7.16-7.19 (m, 1H), 7.45-7.53 (m, 8H), 7.59-7.62 (m, 1H), 7.73-7.75 (m, 1H), 7.83-7.87 (m, 1H), 8.22 (s, 1H). Chiral separation condition: MeOH contained 0.5% NH₄OH; (R,R)-Whelk-O1 (4.6×250 mm, 5 μm); retention time: 6.28 minute.

Compound 191-2: LC-MS (ESI) m/z: 666 [M+H]⁺; ¹H-NMR (CD₃OD, 400 MHz): δ (ppm) 1.71-1.75 (m, 2H), 1.84-1.88 (m, 4H), 2.00-2.08 (m, 2H), 2.21-2.28 (m, 1H), 2.40-2.46 (m, 1H), 3.60-3.65 (m, 1H), 3.90-3.99 (m, 2H), 4.15-4.18 (m, 2H), 4.37-4.41 (m, 2H), 4.85-4.86 (m, 1H), 7.12 (s, 1H), 7.16-7.19 (m, 1H), 7.45-7.53 (m, 8H), 7.59-7.62 (m, 1H), 7.73-7.75 (m, 1H), 7.85-7.88 (m, 1H), 8.20 (s, 1H)). Chiral separation condition: MeOH contained 0.5% NH₄OH; (R,R)-Whelk-O1 (4.6×250 mm, 5 μm); retention time: 7.66 min.

### Example 192

### Synthesis of N—((R)-1-(4′-chloro-[1,1′-biphenyl]-4-yl)-1,1-difluoro-3-((3 aS, 6aS)-hexahydropyrrolo[3,4-b]pyrrol-1 (2H)-yl)-3-oxopropan-2-yl)-6-(cyclopentyloxy) naphthalene-2-sulfonamide trifluoroacetate (Compound 192-1), N—((S)-1-(4′-chloro-[1,1′-biphenyl]-4-yl)-1,1-difluoro-3-((3aR,6aR)-hexahydropyrrolo[3,4-b]pyrrol-1(2H)-yl)-3-oxopropan-2-yl)-6-(cyclopentyloxy)naphthalene-2-sulfonamide trifluoroacetate (Compound 192-2), N—((R)-1-(4′-chloro-[1,1′-biphenyl]-4-yl)-1,1-difluoro-3-((3aR,6aR)-hexahydro pyrrolo[3,4-b]pyrrol-1(2H)-yl)-3-oxopropan-2-yl)-6-(cyclopentyloxy)naphthalene-2-sulfonamide trifluoroacetate (Compound 192-3), and N—((S)-1-(4′-chloro-[1,1′-biphenyl]-4-yl)-1,1-difluoro-3-((3 aS,6aS)-hexahydropyrrolo[3,4-b]pyrrol-1 (2H)-yl)-3-oxopropan-2-yl)-6-(cyclopentyloxy) naphthalene-2-sulfonamide trifluoroacetate (Compound 192-4)

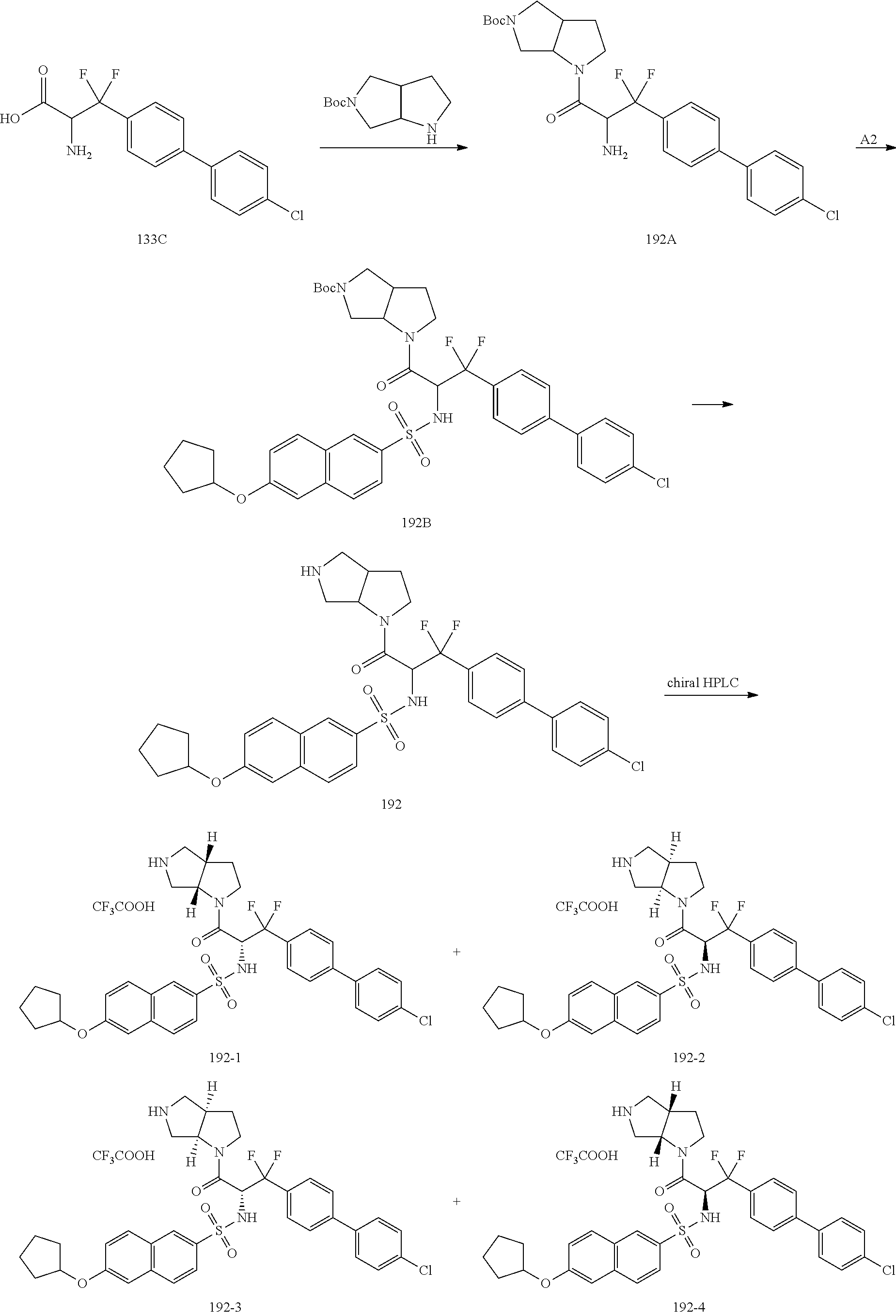

Compounds 192A, 192B, and 192 were synthesized by employing the procedures described for Compound B4, Compounds 2D, and 6 using tert-butyl hexahydropyrrolo[3,4-b]pyrrole-5(1H)-carboxylate, Compounds 133C, 192A, Compound A2, and Compound 192B in lieu of Compounds B4-1, B2-6, B2, Compounds 2C, and 6C.

Compound 192A: LC-MS (ESI) m/z: 506 [M+H]⁺.

Compound 192B was used directly in next step without further purification. LC-MS (ESI) m/z: 780 [M+H]⁺.

Compound 192 was separated with chiral HPLC to give Compound 192-1, Compound 192-2, Compound 192-3, and Compound 192-4.

Compound 192-1: LC-MS (ESI) m/z: 680 [M+H]⁺; ¹H-NMR (CD₃OD, 400 MHz): δ (ppm) 1.70-2.03 (m, 11H), 2.77-2.82 (m, 1H), 2.99-3.09 (m, 2H), 3.40-3.45 (m, 1H), 3.60-3.70 (m, 2H), 3.78-3.85 (m, 1H), 4.02-4.07 (m, 1H), 4.72-4.76 (m, 1H), 7.06 (s, 1H), 7.12-7.15 (m, 1H), 7.44-7.54 (m, 9H), 7.67-7.69 (m, 1H), 7.80-7.83 (m, 1H), 8.13 (s, 1H). Chiral separation condition: MeOH contained 0.5% NH₄OH; (R,R)-Whelk-O1 (4.6×250 mm, 5 μm); retention time: 6.81 minute.

Compound 192-2: LC-MS (ESI) m/z: 680 [M+H]⁺; ¹H-NMR (CD₃OD, 400 MHz): δ (ppm) 1.70-2.04 (m, 11H), 2.76-2.83 (m, 1H), 2.96-3.07 (m, 2H), 3.40-3.45 (m, 1H), 3.60-3.70 (m, 2H), 3.78-3.85 (m, 1H), 4.02-4.07 (m, 1H), 4.72-4.76 (m, 1H), 7.06 (s, 1H), 7.12-7.15 (m, 1H), 7.44-7.54 (m, 9H), 7.67-7.69 (m, 1H), 7.81-7.84 (m, 1H), 8.14 (s, 1H). Chiral separation condition: MeOH contained 0.5% NH₄OH; (R,R)-Whelk-O1 (4.6×250 mm, 5 μm); retention time: 10.33 minute.

Compound 192-3: LC-MS (ESI) m/z: 680 [M+H]⁺; ¹H-NMR (CD₃OD, 400 MHz): δ (ppm) 1.28-1.37 (m, 2H), 1.72-2.03 (m, 8H), 2.18-2.21 (m, 1H), 3.16-3.17 (m, 2H), 3.51-3.57 (m, 1H), 3.67-3.74 (m, 1H), 3.84-4.00 (m, 2H), 4.59-4.73 (m, 3H), 6.92 (s, 1H), 7.05-7.08 (m, 1H), 24-7.45 (m, 9H), 7.57-7.59 (m, 1H), 7.71-7.74 (m, 1H), 8.05 (s, 1H). Chiral separation condition: MeOH contained 0.5% NH₄OH; OD-H (4.6×150 mm, 5 μm); retention time: 3.65 minute.

Compound 192-4: LC-MS (ESI) m/z: 680 [M+H]⁺; ¹H-NMR (CD₃OD, 400 MHz): δ (ppm) 1.29-1.36 (m, 2H), 1.70-2.01 (m, 8H), 2.18-2.21 (m, 1H), 3.16-3.17 (m, 2H), 3.51-3.57 (m, 1H), 3.67-3.74 (m, 1H), 3.84-4.00 (m, 2H), 4.59-4.73 (m, 3H), 6.92 (s, 1H), 7.05-7.08 (m, 1H), 24-7.45 (m, 9H), 7.57-7.59 (m, 1H), 7.73-7.75 (m, 1H), 8.03 (s, 1H). Chiral separation condition: MeOH contained 0.5% NH₄OH; OD-H (4.6×150 mm, 5 μm); retention time: 2.52 minute.

### Example 193

### Synthesis of (R)—N-(3-(4-aminopiperidin-1-yl)-1,1-difluoro-3-oxo-1-(4-(3,3,3-trifluoroprop-1-en-2-yl)phenyl)propan-2-yl)-4-(cyclohexylmethoxy) benzenesulfonamide (Compound 193-1), and (S)—N-(3-(4-aminopiperidin-1-yl)-1,1-difluoro-3-oxo-1-(4-(3,3,3-trifluoroprop-1-en-2-yl)phenyl)propan-2-yl)-4-(cyclohexylmethoxy)benzenesulfonamide (Compound 193-2)

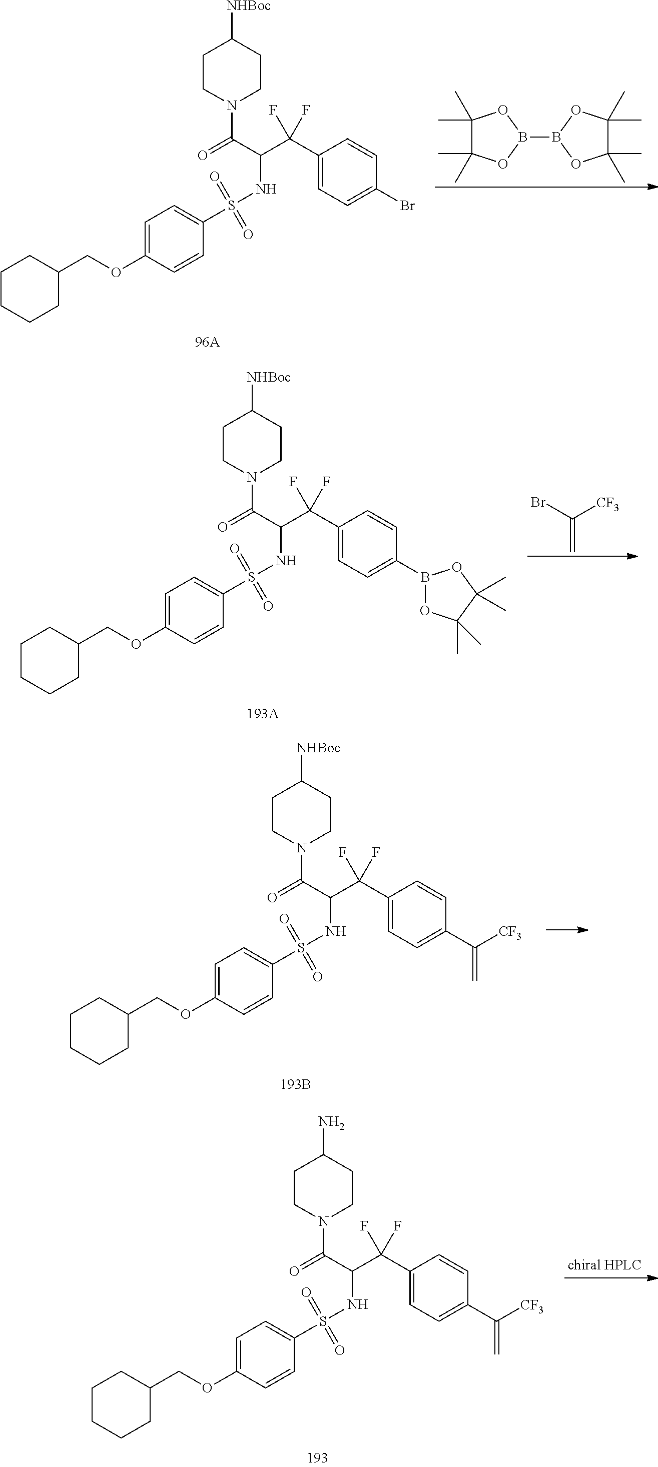

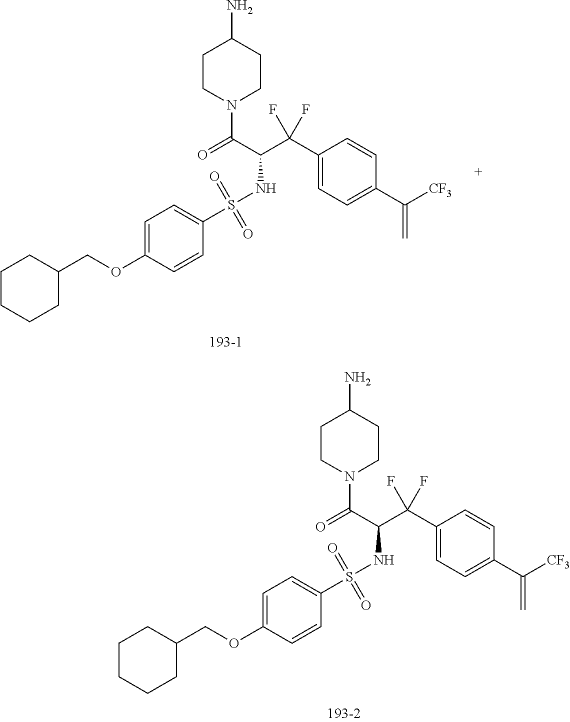 [SMILES: C=C(c1ccc(C(F)(F)[C@H](NS(=O)(=O)c2ccc(OCC3CCCCC3)cc2)C(=O)N2CCC(N)CC2)cc1)C(F)(F)F ; C=C(c1ccc(C(F)(F)[C@@H](NS(=O)(=O)c2ccc(OCC3CCCCC3)cc2)C(=O)N2CCC(N)CC2)cc1)C(F)(F)F]

Compounds 193A, 193B, and 193 were synthesized by employing the procedures described for Compounds 166A, 166B, and 6 using Compounds 96A, 193A, and 193B in lieu of Compounds 32A, 166A, and 6C.

Compound 193A: LC-MS (ESI) m/z: 706 [M+H−56]⁺.

Compound 193B: LC-MS (ESI) m/z: 730 [M+H]⁺.

Compound 193 was separated with chiral HPLC to furnish Compound 193-1 and Compound 193-2.

Compound 193-1: LC-MS (ESI) m/z: 630 [M+H]⁺; ¹H-NMR (CD₃OD, 400 MHz): δ (ppm) 0.78-1.15 (m, 6H), 1.22-1.31 (m, 2H), 1.61-1.82 (m, 8H), 2.13-2.97 (m, 3H), 3.71-4.20 (m, 4H), 5.92-5.99 (m, 2H), 6.84-6.87 (m, 2H), 7.41-7.57 (m, 6H). Chiral separation condition: MeOH contained 0.5% NH₄OH; OZ-H (4.6×250 mm, 5 μm); retention time: 5.91 minute.

Compound 193-2: LC-MS (ESI) m/z: 630 [M+H]⁺; ¹H-NMR (CD₃OD, 400 MHz): δ (ppm) 0.80-1.25 (m, 7H), 1.32-1.41 (m, 1H), 1.71-1.90 (m, 8H), 2.13-2.97 (m, 3H), 3.83-4.20 (m, 4H), 6.02-6.10 (m, 2H), 6.95-7.00 (m, 2H), 7.52-7.66 (m, 6H). Chiral separation condition: MeOH contained 0.5% NH₄OH; OZ-H (4.6×250 mm, 5 μm); retention time: 8.74 minute.

### Example 194

### Synthesis of N-((2R)-3-(3-amino-8-azabicyclo[3.2.1]octan-8-yl)-1-(5-chlorothiophen-2-yl)-1,1-difluoro-3-oxopropan-2-yl)-4-(cyclohexylmethoxy)benzenesulfonamide (Compound 194-1), and N-((2S)-3-(3-amino-8-azabicyclo[3.2.1]octan-8-yl)-1-(5-chlorothiophen-2-yl)-1,1-difluoro-3-oxopropan-2-yl)-4-(cyclohexylmethoxy) benzenesulfonamide (Compound 194-2)

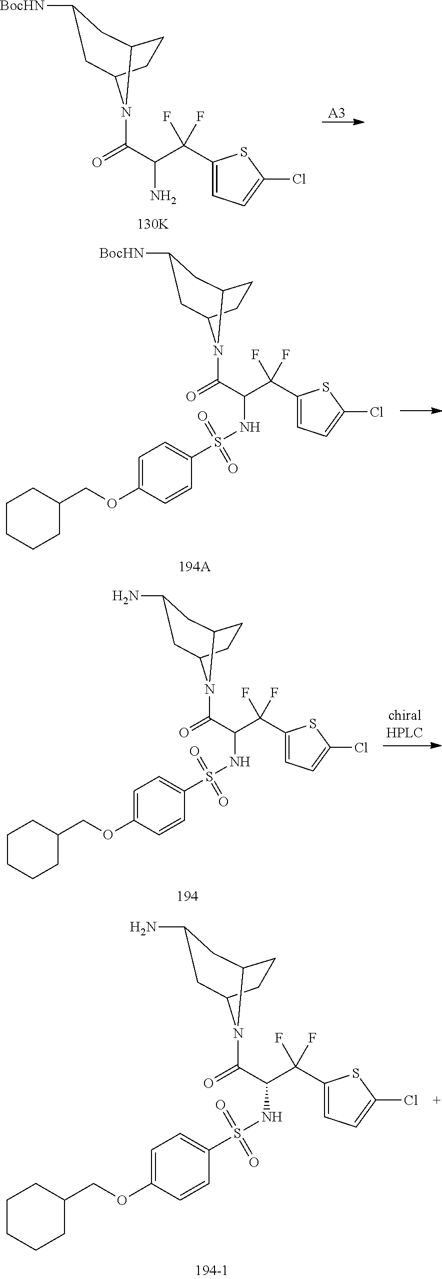 [SMILES: CC(C)(C)OC(=O)NC1CC2CCC(C1)N2C(=O)C(N)C(F)(F)c1ccc(Cl)s1 ; "A3" ; CC(C)(C)OC(=O)NC1CC2CCC(C1)N2C(=O)C(NS(=O)(=O)c1ccc(OCC2CCCCC2)cc1)C(F)(F)c1ccc(Cl)s1 ; NC1CC2CCC(C1)N2C(=O)C(NS(=O)(=O)c1ccc(OCC2CCCCC2)cc1)C(F)(F)c1ccc(Cl)s1 ; NC1CC2CCC(C1)N2C(=O)[C@@H](NS(=O)(=O)c1ccc(OCC2CCCCC2)cc1)C(F)(F)c1ccc(Cl)s1]

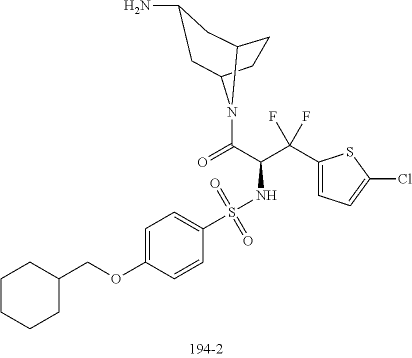 [SMILES: NC1CC2CCC(C1)N2C(=O)[C@H](NS(=O)(=O)c1ccc(OCC2CCCCC2)cc1)C(F)(F)c1ccc(Cl)s1]

Compounds 194A and 194 were synthesized by employing the procedures described for Compounds 1A and 6 using Compound A3, Compounds 130K, and 194A in lieu of Compounds A1, B2, and Compound 6C.

Compound 194A was used directly in the next step without further purification. LC-MS (ESI) m/z: 702 [M+H]⁺.

Compound 194 was separated by chiral HPLC to furnish Compound 194-1 and Compound 194-2.

Compound 194-1: LC-MS (ESI) m/z: 602 [M+H]⁺; ¹H-NMR (CD₃OD, 400 MHz): δ (ppm) 0.81-1.26 (m, 8H), 1.54-1.82 (m, 11H), 2.88-3.07 (m, 1H), 3.73-3.76 (m, 2H), 4.20-4.63 (m, 3H), 6.82-6.92 (m, 3H), 7.04-7.11 (m, 1H), 7.58-7.65 (m, 2H). Chiral separation condition: MeOH contained 0.5% NH₄OH; OJ (4.6×250 mm, 5 μm); retention time: 3.92 minute.

Compound 194-2: LC-MS (ESI) m/z: 602 [M+H]⁺; ¹H-NMR (CD₃OD, 400 MHz): δ (ppm) 0.80-1.26 (m, 8H), 1.41-1.82 (m, 11H), 2.88-3.07 (m, 1H), 3.73-3.75 (m, 2H), 4.21-4.63 (m, 3H), 6.81-6.92 (m, 3H), 7.03-7.09 (m, 1H), 7.56-7.64 (m, 2H). Chiral separation condition: MeOH contained 0.5% NH₄OH; OJ (4.6×250 mm, 5 μm); retention time: 2.6 minute.

### Example 195

### Synthesis of N—((R)-1-(4′-chloro-[1,1′-biphenyl]-4-yl)-1,1-difluoro-3-((3 aS, 6aS)-hexahydropyrrolo[3,4-b]pyrrol-1 (2H)-yl)-3-oxopropan-2-yl)-4-(cyclohexylmethoxy) benzenesulfonamide trifluoroacetate (Compound 195-1), N—((S)-1-(4′-chloro-[1,1′-biphenyl]-4-yl)-1,1-difluoro-3-((3aR,6aR)-hexahydropyrrolo[3,4-b]pyrrol-1 (2H)-yl)-3-oxopropan-2-yl)-4-(cyclohexylmethoxy)benzenesulfonamide trifluoroacetate (Compound 195-2), N—((R)-1-(4′-chloro-[1,1′-biphenyl]-4-yl)-1,1-difluoro-3-((3aR,6aR)-hexahydropyrrolo[3,4-b]pyrrol-1(2H)-yl)-3-oxopropan-2-yl)-4-(cyclohexylmethoxy)benzenesulfonamide trifluoroacetate (Compound 195-3), and N—((S)-1-(4′-chloro-[1,1′-biphenyl]-4-yl)-1,1-difluoro-3-((3aS,6aS)-hexahydropyrrolo [3,4-b]pyrrol-1(2H)-yl)-3-oxopropan-2-yl)-4-(cyclohexylmethoxy) benzenesulfonamide trifluoroacetate (Compound 195-4)

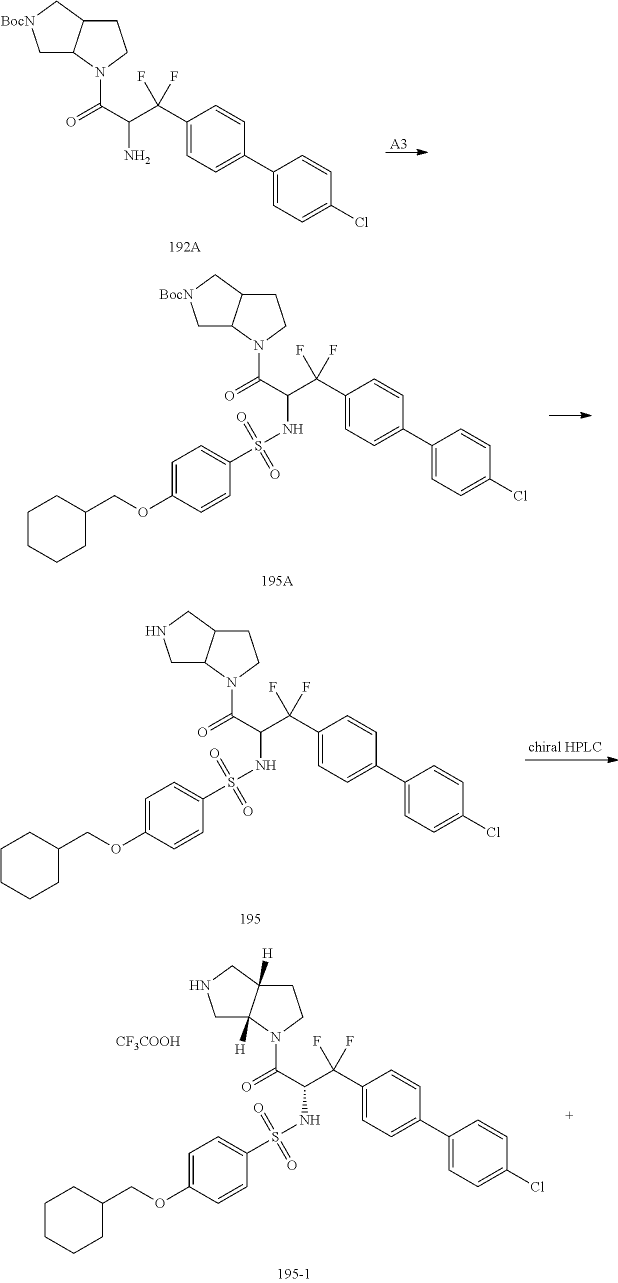 [SMILES: CC(C)(C)OC(=O)N1CC2CCN(C(=O)C(N)C(F)(F)c3ccc(-c4ccc(Cl)cc4)cc3)C2C1 ; "A3" ; CC(C)(C)OC(=O)N1CC2CCN(C(=O)C(NS(=O)(=O)c3ccc(OCC4CCCCC4)cc3)C(F)(F)c3ccc(-c4ccc(Cl)cc4)cc3)C2C1 ; O=C(C(NS(=O)(=O)c1ccc(OCC2CCCCC2)cc1)C(F)(F)c1ccc(-c2ccc(Cl)cc2)cc1)N1CCC2CNCC21 ; O=C([C@@H](NS(=O)(=O)c1ccc(OCC2CCCCC2)cc1)C(F)(F)c1ccc(-c2ccc(Cl)cc2)cc1)N1CC[C@H]2CNC[C@H]21 ; "CF3COOH"]

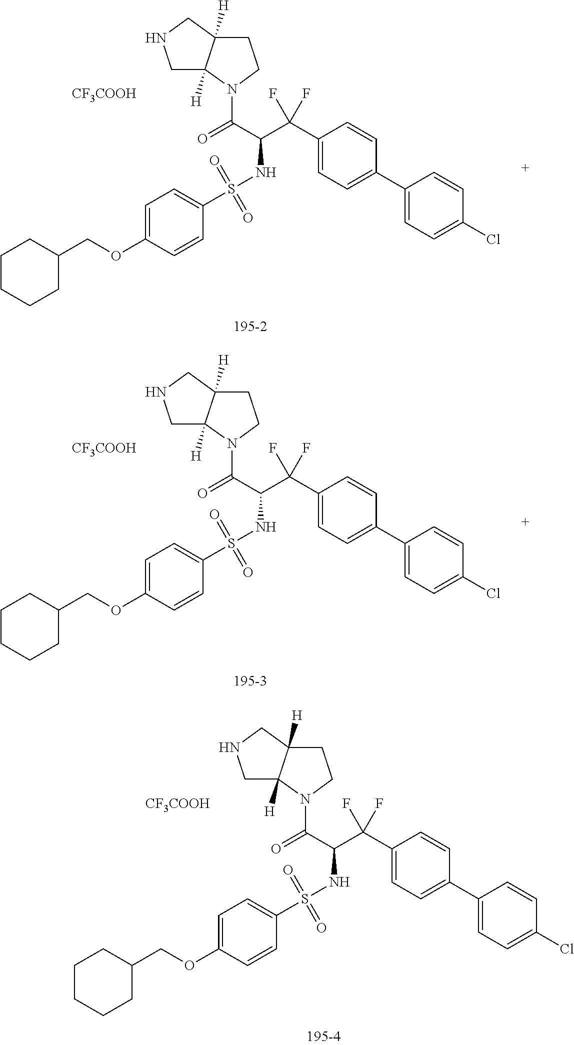 [SMILES: O=C([C@H](NS(=O)(=O)c1ccc(OCC2CCCCC2)cc1)C(F)(F)c1ccc(-c2ccc(Cl)cc2)cc1)N1CC[C@@H]2CNC[C@@H]21 ; "CF3COOH" ; O=C([C@@H](NS(=O)(=O)c1ccc(OCC2CCCCC2)cc1)C(F)(F)c1ccc(-c2ccc(Cl)cc2)cc1)N1CC[C@@H]2CNC[C@@H]21 ; "CF3COOH" ; O=C([C@H](NS(=O)(=O)c1ccc(OCC2CCCCC2)cc1)C(F)(F)c1ccc(-c2ccc(Cl)cc2)cc1)N1CC[C@H]2CNC[C@H]21 ; "CF3COOH"]

Compounds 195A and 195 were synthesized by employing the procedures described for Compounds 2D and 6 using Compound A3, Compounds 192A, and 195A in lieu of Compound 2C, Compound B2, and Compound 6C.

Compound 195A was used directly in next step without further purification. LC-MS (ESI) m/z: 758 [M+H]⁺.

Compound 195 was separated with chiral HPLC to give Compound 195-1, Compound 195-2, Compound 195-3, and Compound 195-4.

Compound 195-1: LC-MS (ESI) m/z: 658 [M+H]⁺; ¹H-NMR (CD₃OD, 400 MHz): δ (ppm) 0.96-0.99 (m, 2H), 1.27-1.33 (m, 4H), 1.66-1.78 (m, 6H), 2.02-2.22 (m, 2H), 3.10-3.18 (m, 3H), 3.52-3.58 (m, 2H), 3.67-3.72 (m, 2H), 3.78-3.85 (m, 1H), 4.37-4.38 (m, 1H), 4.63-4.69 (m, 1H), 6.79 (m, 2H), 7.44-7.54 (m, 6H), 7.59-7.70 (m, 4H). Chiral separation condition: MeOH contained 0.2% methanol ammonia; (R,R)-Whelk-O1 (4.6×250 mm, 5 μm); retention time: 5.71 minute.

Compound 195-2: LC-MS (ESI) m/z: 658 [M+H]⁺; ¹H-NMR (CD₃OD, 400 MHz): δ (ppm) 0.95-0.99 (m, 2H), 1.28-1.33 (m, 4H), 1.65-1.78 (m, 6H), 2.02-2.22 (m, 2H), 3.10-3.18 (m, 3H), 3.52-3.58 (m, 2H), 3.67-3.72 (m, 2H), 3.78-3.85 (m, 1H), 4.37-4.38 (m, 1H), 4.63-4.69 (m, 1H), 6.79 (m, 2H), 7.44-7.54 (m, 6H), 7.58-7.72 (m, 4H). Chiral separation condition: MeOH contained 0.2% methanol ammonia; (R,R)-Whelk-O1 (4.6×250 mm, 5 μm); retention time: 7.11 minute.

Compound 195-3: LC-MS (ESI) m/z: 658 [M+H]⁺; ¹H-NMR (CD₃OD, 400 MHz): δ (ppm) 0.91-0.94 (m, 2H), 1.27-1.33 (m, 4H), 1.65-1.78 (m, 6H), 2.02-2.22 (m, 2H), 3.18-3.20 (m, 3H), 3.52-3.58 (m, 2H), 3.60-3.71 (m, 1H), 3.82-3.91 (m, 2H), 4.58-4.63 (m, 2H), 6.72-6.75 (m, 2H), 7.40-7.45 (m, 4H), 7.50-7.54 (m, 4H), 7.66-7.68 (m, 2H). Chiral separation condition: MeOH contained 0.2% methanol ammonia; IC (4.6×150 mm, 5 μm); retention time: 4.59 minute.

Compound 195-4: LC-MS (ESI) m/z: 658 [M+H]⁺; ¹H-NMR (CD₃OD, 400 MHz): δ (ppm) 0.92-0.95 (m, 2H), 1.28-1.36 (m, 4H), 1.65-1.78 (m, 6H), 2.02-2.22 (m, 2H), 3.18-3.20 (m, 3H), 3.52-3.58 (m, 2H), 3.60-3.71 (m, 1H), 3.82-3.91 (m, 2H), 4.58-4.63 (m, 2H), 6.72-6.75 (m, 2H), 7.40-7.45 (m, 4H), 7.50-7.54 (m, 4H), 7.68-7.74 (m, 2H). Chiral separation condition: MeOH contained 0.2% methanol ammonia; IC (4.6×150 mm, 5 μm); retention time: 3.28 minute.

### Example 196

### Synthesis of (R)—N-(3-(4-aminopiperidin-1-yl)-1,1-difluoro-3-oxo-1-(4-(1-(trifluoromethyl)cyclopropyl)phenyl)propan-2-yl)-4-(cyclohexylmethoxy) benzenesulfonamide trifluoroacetate (Compound 196-1), and (S)—N-(3-(4-aminopiperidin-1-yl)-1,1-difluoro-3-oxo-1-(4-(1-(trifluoromethyl)cyclopropyl) phenyl)propan-2-yl)-4-(cyclohexylmethoxy)benzenesulfonamide trifluoroacetate (Compound 196-2)

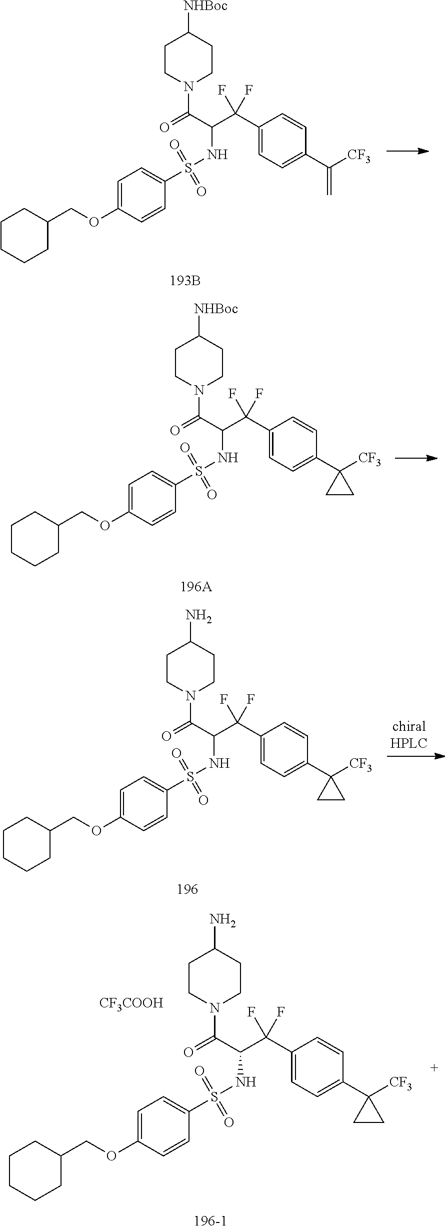 [SMILES: C=C(c1ccc(C(F)(F)C(NS(=O)(=O)c2ccc(OCC3CCCCC3)cc2)C(=O)N2CCC(NC(=O)OC(C)(C)C)CC2)cc1)C(F)(F)F ; CC(C)(C)OC(=O)NC1CCN(C(=O)C(NS(=O)(=O)c2ccc(OCC3CCCCC3)cc2)C(F)(F)c2ccc(C3(C(F)(F)F)CC3)cc2)CC1 ; NC1CCN(C(=O)C(NS(=O)(=O)c2ccc(OCC3CCCCC3)cc2)C(F)(F)c2ccc(C3(C(F)(F)F)CC3)cc2)CC1 ; NC1CCN(C(=O)[C@@H](NS(=O)(=O)c2ccc(OCC3CCCCC3)cc2)C(F)(F)c2ccc(C3(C(F)(F)F)CC3)cc2)CC1 ; "CF3COOH"]

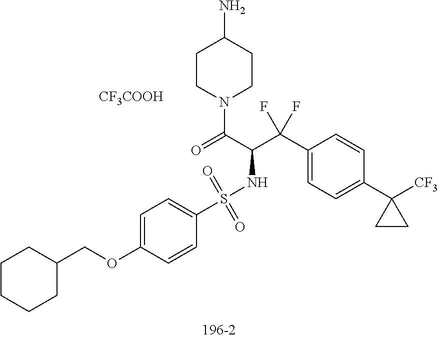 [SMILES: NC1CCN(C(=O)[C@H](NS(=O)(=O)c2ccc(OCC3CCCCC3)cc2)C(F)(F)c2ccc(C3(C(F)(F)F)CC3)cc2)CC1 ; "CF3COOH"]

Compounds 196A and 196 were synthesized by employing the procedures described for Compounds 182A and 6 using Compounds 193B, and 196A in lieu of Compounds 166B and 6C.

Compound 196A: LC-MS (ESI) m/z: 766 [M+Na]⁺.

Compound 196 was separated with chiral-HPLC to afford Compound 196-1 and Compound 196-2.

Compound 196-1: LC-MS (ESI) m/z: 644 [M+H]⁺; ¹H-NMR (CD₃OD, 400 MHz): δ (ppm) 1.11-2.10 (m, 20H), 2.38-3.07 (m, 2H), 3.84-3.86 (m, 2H), 4.18-4.45 (m, 2H), 4.94-4.96 (m, 1H), 6.92-6.97 (m, 2H), 7.42-7.62 (m, 6H). Chiral separation condition: n-Hexane/EtOH contained 0.1% DEA, 90/10; AS-H (4.6×250 mm, 5 μm); retention time 20.82 minute.

Compound 196-2: LC-MS (ESI) m/z: 644 [M+H]⁺; ¹H-NMR (CD₃OD, 400 MHz): δ (ppm) 1.08-2.15 (m, 20H), 2.40-3.08 (m, 2H), 3.84-3.86 (m, 2H), 4.16-4.35 (m, 2H), 4.96-5.06 (m, 1H), 6.92-6.97 (m, 2H), 7.42-7.61 (m, 6H). Chiral separation condition: n-Hexane/EtOH contained 0.1% DEA, 90/10; AS-H (4.6×250 mm, 5 μm); retention time: 12.12 minute.

### Example 197

### Synthesis of N—((R)-1-(4′-chloro-2-methoxy-[1,1′-biphenyl]-4-yl)-1,1-difluoro-3-((3aR,6aS)-hexahydropyrrolo[3,4-c]pyrrol-2(1H)-yl)-3-oxopropan-2-yl)-6-(cyclopentyloxy)naphthalene-2-sulfonamide (Compound 197-1), and N—((S)-1-(4′-chloro-2-methoxy-[1,1′-biphenyl]-4-yl)-1,1-difluoro-3-((3aR,6aS)-hexahydropyrrolo [3,4-c]pyrrol-2(1H)-yl)-3-oxopropan-2-yl)-6-(cyclopentyloxy)naphthalene-2-sulfonamide (Compound 197-2)

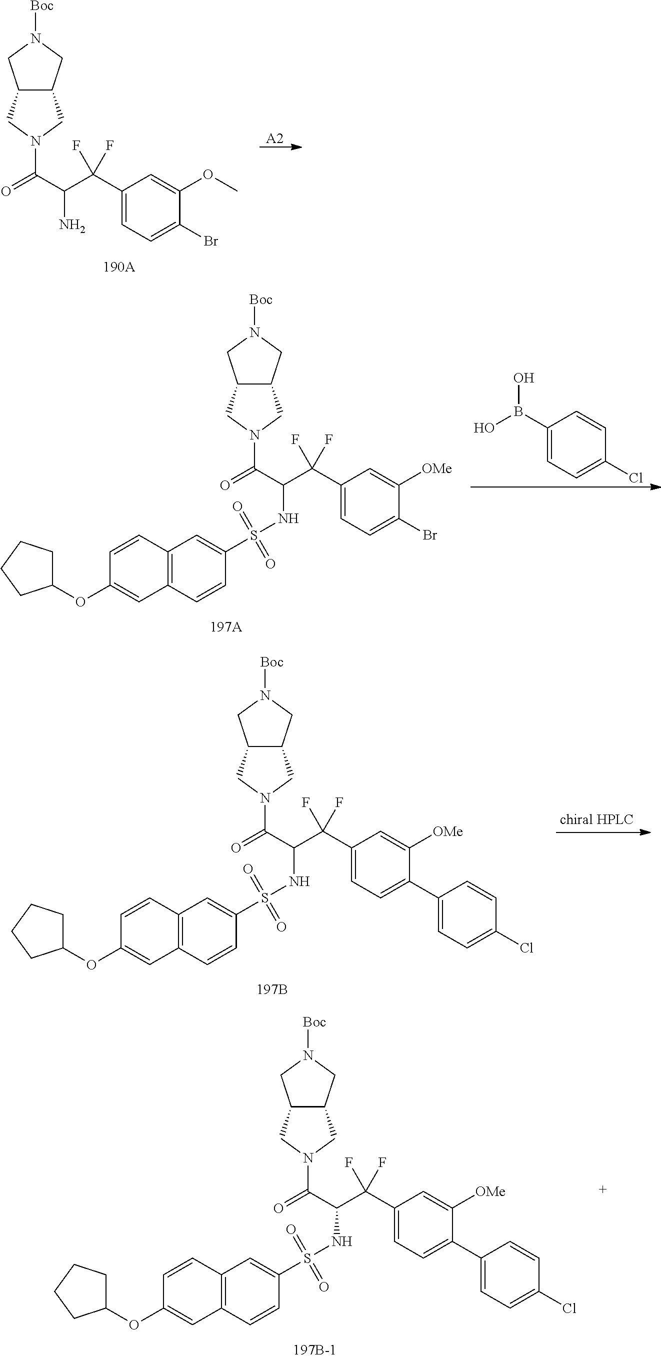 [SMILES: COc1cc(C(F)(F)C(N)C(=O)N2C[C@H]3CN(C(=O)OC(C)(C)C)C[C@H]3C2)ccc1Br ; "A2" ; COc1cc(C(F)(F)C(NS(=O)(=O)c2ccc3cc(OC4CCCC4)ccc3c2)C(=O)N2C[C@H]3CN(C(=O)OC(C)(C)C)C[C@H]3C2)ccc1Br ; OB(O)c1ccc(Cl)cc1 ; COc1cc(C(F)(F)C(NS(=O)(=O)c2ccc3cc(OC4CCCC4)ccc3c2)C(=O)N2C[C@H]3CN(C(=O)OC(C)(C)C)C[C@H]3C2)ccc1-c1ccc(Cl)cc1 ; COc1cc(C(F)(F)[C@H](NS(=O)(=O)c2ccc3cc(OC4CCCC4)ccc3c2)C(=O)N2C[C@H]3CN(C(=O)OC(C)(C)C)C[C@H]3C2)ccc1-c1ccc(Cl)cc1]

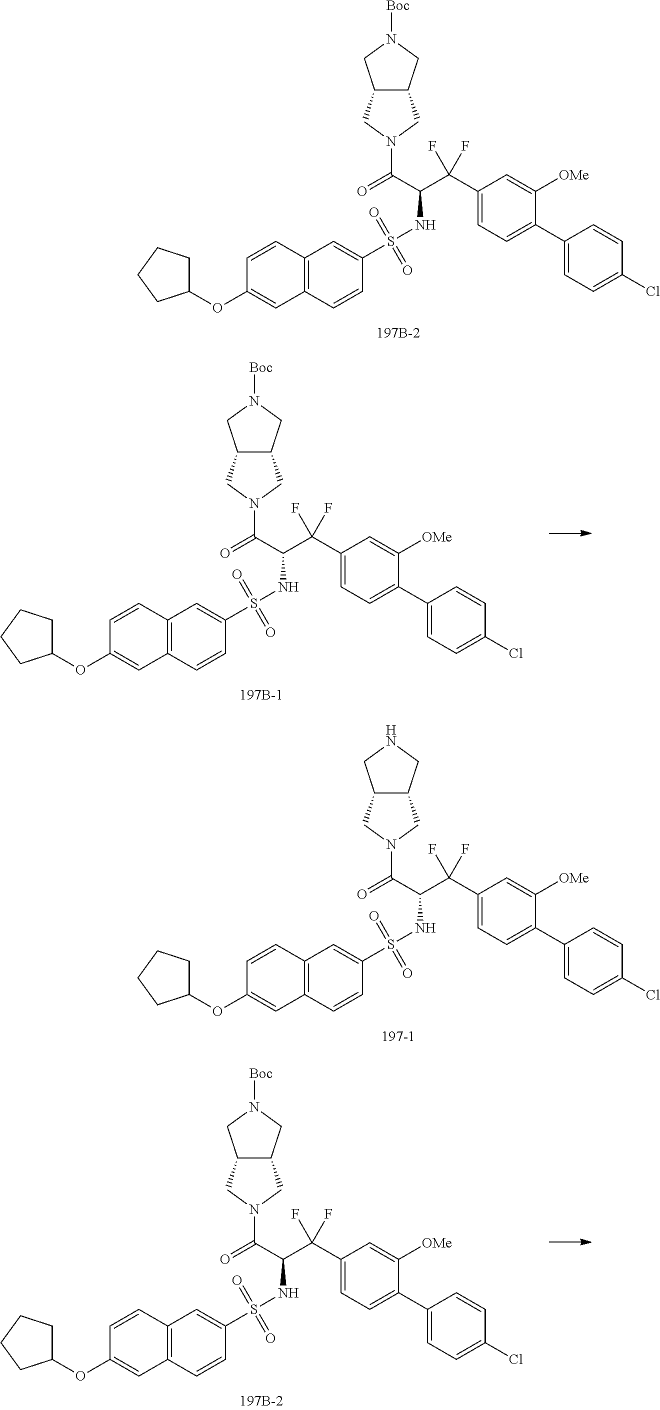 [SMILES: COc1cc(C(F)(F)[C@@H](NS(=O)(=O)c2ccc3cc(OC4CCCC4)ccc3c2)C(=O)N2C[C@H]3CN(C(=O)OC(C)(C)C)C[C@H]3C2)ccc1-c1ccc(Cl)cc1 ; COc1cc(C(F)(F)[C@H](NS(=O)(=O)c2ccc3cc(OC4CCCC4)ccc3c2)C(=O)N2C[C@H]3CN(C(=O)OC(C)(C)C)C[C@H]3C2)ccc1-c1ccc(Cl)cc1 ; COc1cc(C(F)(F)[C@H](NS(=O)(=O)c2ccc3cc(OC4CCCC4)ccc3c2)C(=O)N2C[C@H]3CNC[C@H]3C2)ccc1-c1ccc(Cl)cc1 ; COc1cc(C(F)(F)[C@@H](NS(=O)(=O)c2ccc3cc(OC4CCCC4)ccc3c2)C(=O)N2C[C@H]3CN(C(=O)OC(C)(C)C)C[C@H]3C2)ccc1-c1ccc(Cl)cc1]

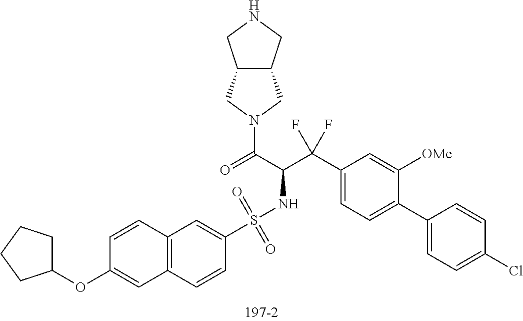 [SMILES: COc1cc(C(F)(F)[C@@H](NS(=O)(=O)c2ccc3cc(OC4CCCC4)ccc3c2)C(=O)N2C[C@H]3CNC[C@H]3C2)ccc1-c1ccc(Cl)cc1]

Compounds 197A and 197B were synthesized by employing the procedures described for Compounds 2D and 14C using Compound A2, Compounds 190A, 197A, and 4-chlorophenylboronic acid in lieu of Compound 2C, Compound B2, Compounds 14A, and 14B.

Compound 197A: LC-MS (ESI) m/z: 722 [M−55]⁺.

Compound 197B was separated with chiral HPLC to afford Compound 197B-1, LC-MS (ESI) m/z: 754 [M−55]⁺, and Compound 197B-2, LC-MS (ESI) m/z: 754 [M−55]⁺.

Compound 197-1 was synthesized by employing the procedure described for Compound 1 using Compound 197B-1 in lieu of Compound 1A.

LC-MS (ESI) m/z: 710 [M+H]⁺; ¹H-NMR (DMSO-d₆, 400 MHz): δ (ppm) 1.62-1.73 (m, 6H), 2.00-1.99 (m, 2H), 2.65-3.34 (m, 7H), 3.55-3.78 (m, 6H), 4.66-4.89 (m, 2H), 7.01-7.32 (m, 6H), 7.38-7.56 (m, 4H), 7.78 (t, J=2.8 Hz, 1H), 7.95 (t, J=2.8 Hz, 1H), 8.15-8.19 (m, 1H), 8.76-8.87 (m, 1H), 9.27-9.41 (m, 2H). Chiral separation condition: MeOH contained 0.5% NH₄OH; (R,R)-whelk-O1 (4.6×250 mm, 5 μm); retention time: 7.36 minute.

Compound 197-2 was synthesized by employing the procedure described for Compound 1 using Compound 197B-2 in lieu of Compound 1A, as a pale-yellow solid (yield 53%). LC-MS (ESI) m/z: 710 [M+H]⁺; ¹H-NMR (DMSO-d₆, 400 MHz): δ (ppm) 1.62-1.73 (m, 6H), 1.99 (s, 2H), 2.66-3.34 (m, 7H), 3.55-3.78 (m, 6H), 4.65-4.88 (m, 2H), 7.01-7.32 (m, 6H), 7.38-7.56 (m, 4H), 7.78 (d, J=8.8, 1H), 7.95 (t, J=2.8, 1H), 8.15-8.19 (m, 1H), 8.78-8.87 (m, 1H), 9.28-9.4 (m, 2H). Chiral separation condition: MeOH contained 0.5% NH₄OH; (R,R)-whelk-O1 (4.6×250 mm, 5 μm); retention time: 9.11 minute.

### Example 198

### Synthesis of (R)—N-(3-(4-aminopiperidin-1-yl)-1-(7-chlorodibenzo[b,d]furan-3-yl)-1,1-difluoro-3-oxopropan-2-yl)-4-(cyclohexylmethoxy)benzenesulfonamide (Compound 198-1), and (S)—N-(3-(4-aminopiperidin-1-yl)-1-(7-chlorodibenzo [b,d]furan-3-yl)-1,1-difluoro-3-oxopropan-2-yl)-4-(cyclohexylmethoxy) benzenesulfonamide (Compound 198-2)

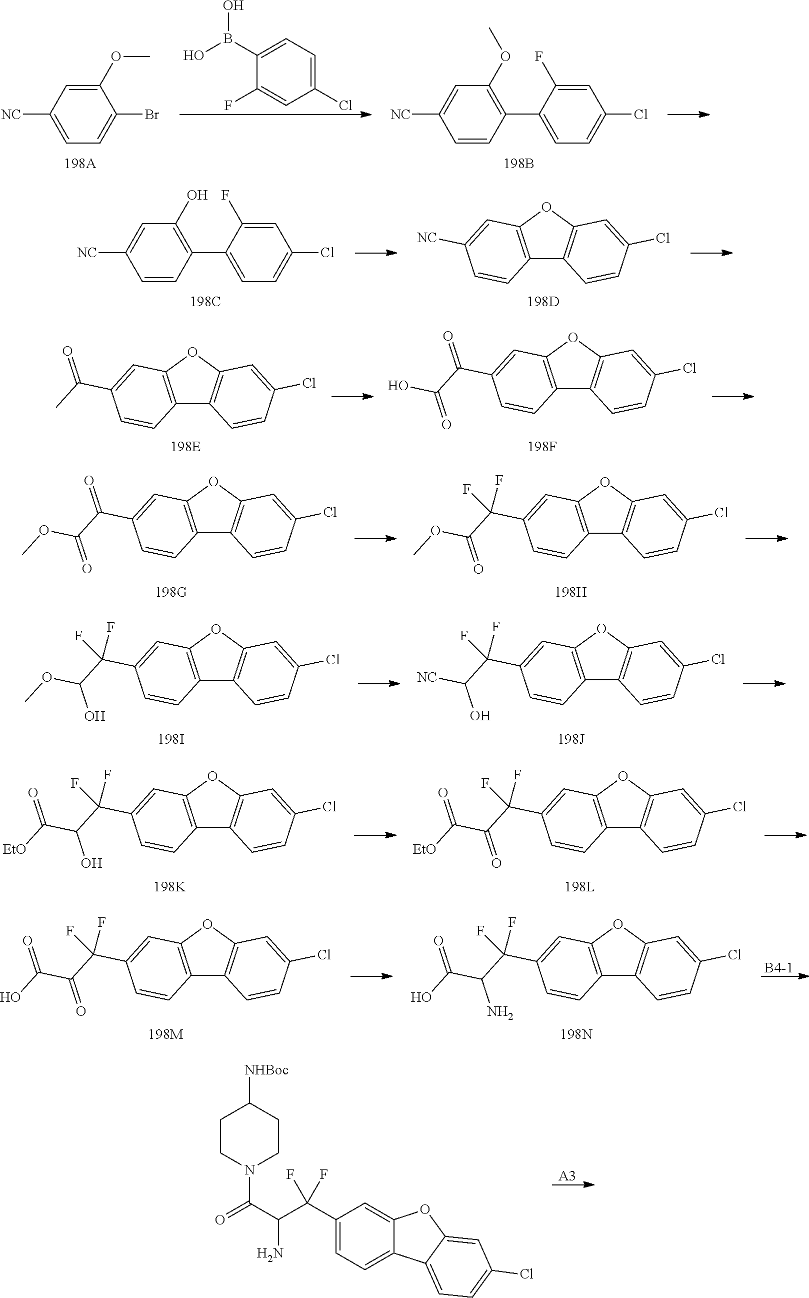

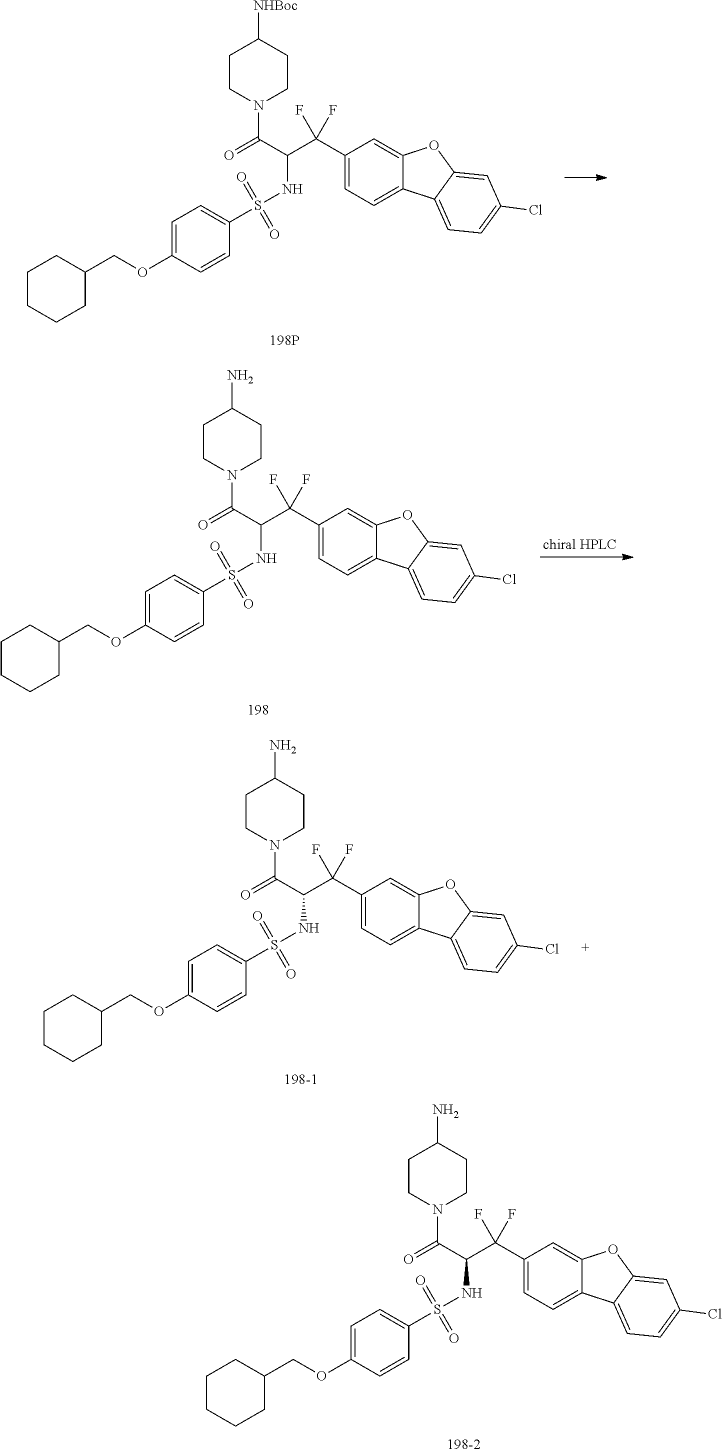 [SMILES: CC(C)(C)OC(=O)NC1CCN(C(=O)C(NS(=O)(=O)c2ccc(OCC3CCCCC3)cc2)C(F)(F)c2ccc3c(c2)oc2cc(Cl)ccc23)CC1 ; NC1CCN(C(=O)C(NS(=O)(=O)c2ccc(OCC3CCCCC3)cc2)C(F)(F)c2ccc3c(c2)oc2cc(Cl)ccc23)CC1 ; NC1CCN(C(=O)[C@@H](NS(=O)(=O)c2ccc(OCC3CCCCC3)cc2)C(F)(F)c2ccc3c(c2)oc2cc(Cl)ccc23)CC1 ; NC1CCN(C(=O)[C@H](NS(=O)(=O)c2ccc(OCC3CCCCC3)cc2)C(F)(F)c2ccc3c(c2)oc2cc(Cl)ccc23)CC1]

To a solution of 4-chloro-2-fluorophenylboronic acid (20 g, 114 mmol) in 1,4-dioxane and water (400 mL/40 mL, v/v) was added sodium carbonate (36 g, 342 mmol), Pd(Ph₃P)₄ (7.0 g, 6.0 mmol), and 4-bromo-3-methoxybenzonitrile, Compound 198A, (24 g, 114 mmol) under nitrogen atmosphere. The mixture was stirred under nitrogen at 90° C. for 16 hours. The reaction mixture was concentrated under reduced pressure and the residue was purified by silica gel column chromatography to give Compound 198B. LC-MS (ESI) m/z: 262 [M+H]⁺; ¹H-NMR (CDCl₃, 400 MHz): δ (ppm) 3.84 (s, 3H), 7.17-7.26 (m, 4H), 7.33-7.35 (m, 2H).

A solution of Compound 198B (20 g, 77 mmol) and BBr₃ (57 g, 231 mmol) in dichloromethane (500 mL) was stirred at room temperature overnight. The reaction mixture was quenched with saturated aqueous NaHCO₃ solution (1000 mL) and extracted with dichloromethane (500 mL×2). The combined organic extracts were washed with brine (300 mL), dried over anhydrous sodium sulfate, filtered, and concentrated to afford a crude product. The crude product was purified by silica gel column chromatography to yield Compound 198C. ¹H-NMR (CDCl₃, 400 MHz): δ (ppm) 6.69 (s, 1H), 7.24-7.34 (m, 6H).

A mixture of Compound 198C (18 g, 73 mmol) and K₂CO₃ (30 g, 219 mmol) in 1-methylpyrrolidin-2-one (200 mL) was heated to 140° C. for 5 hours. After cooled down to room temperature, the reaction mixture was concentrated and purified by silica gel column chromatography to afford Compound 198D. ¹H-NMR (CDCl₃, 400 MHz): δ (ppm) 7.33-7.36 (m, 1H), 7.57-7.60 (m, 2H), 7.80-7.86 (m, 2H), 7.93-7.51 (m, 1H).

To a stirred solution of Compound 198D (17.5 g, 77 mmol) in toluene (400 mL) was added dropwise a solution of methylmagnesium bromide in diethyl ether (3 M, 77 mL, 231 mmol) at 0° C. The reaction mixture was stirred at 80° C. for 5 hours, cooled down to room temperature, quenched with ice-water (300 mL), and filtered. The filtrate was extracted with dichloromethane (200 mL×3). The combined organic extracts were dried over anhydrous sodium sulfate, filtered, and concentrated. The residue was purified by silica gel column chromatography to give Compound 198E. LC-MS (ESI) m/z: 245 [M+H]⁺; ¹H-NMR (CDCl₃, 400 MHz): δ (ppm) 2.71 (s, 3H), 7.36-7.40 (m, 2H), 7.63 (s, 1H), 7.89-8.00 (m, 2H), 8.16 (s, 1H).

Compound 198F was synthesized by employing the procedure described for Compound 42B using Compound 198E in lieu of Compound 42A. LC-MS (ESI) m/z: 275 [M+H]⁺.

To a solution of Compound 198F (16 g, 58 mmol) and Et₃N (11.7 g, 116 mmol) in dichloromethane (200 mL) was dropped methyl carbonochloridate (8.3 g, 87 mmol) at 0° C. The reaction mixture was stirred at 0° C. for 2 hours, poured into crash ice with stirring slowly, and extracted with dichloromethane (100 mL×2). The combined organic extracts was washed with water (100 mL×2) and brine (200 mL), dried over anhydrous sodium sulfate, filtered, and concentrated. The residue was purified by silica gel column chromatography to yield Compound 198G.

Compounds 198H, 198I, 198J, 198K, 198L, 198M, 198N, 1980, 198P, and 198 were synthesized by employing the procedures described for Compound B2-3, Compounds 128G, 128H, 45G, Compound B6-3, Compounds 451, 80A, Compound B4, Compounds 2D, and 1 using Compounds 198G, 198H, 198I, 198J, 198K, 198L, 198M, 198N, 1980, Compound A3, and Compound 198P in lieu of Compound B2-2, Compounds 128F, 128G, 45F, Compound B6-2, Compounds 45H, 51J, Compounds B2-6, B2, Compounds 2C, and 1A.

Compound 198H: ¹H-NMR (CDCl₃, 400 MHz): δ (ppm) 3.87 (s, 3H), 7.36-7.39 (m, 1H), 7.59-7.63 (m, 2H), 7.83 (s, 1H), 7.89 (d, J=7.6 Hz, 1H), 7.99 (d, J=8.0 Hz, 1H).

Compound 1981: LC-MS (ESI) m/z: non-ionizable compound under routine conditions used.

Compound 198J: ¹H-NMR (CDCl₃, 400 MHz): δ (ppm) 3.02 (s, 1H), 4.90-4.93 (m, 1H), 7.38-7.40 (m, 1H), 7.58-7.64 (m, 2H), 7.81 (s, 1H), 7.91 (d, J=8.0 Hz, 1H), 8.04 (d, J=7.6 Hz, 1H).

Compound 198K: ¹H-NMR (CDCl₃, 400 MHz): δ (ppm) 1.28 (t, J=7.2 Hz, 3H), 3.29 (d, J=7.6 Hz, 1H), 4.28-4.32 (m, 2H), 4.58-4.62 (m, 1H), 7.37 (d, J=8.4 Hz, 1H), 7.50 (d, J=8.4 Hz, 1H), 7.61 (s, 1H), 7.72 (s, 1H), 7.89 (d, J=8.0 Hz, 1H), 7.97 (d, J=8.0 Hz, 1H).

Compound 198L: LC-MS (ESI) m/z: 393 [M+Na]⁺. ¹H-NMR (CDCl₃, 400 MHz): δ (ppm) 1.40 (t, J=7.2 Hz, 3H), 4.07 (s, 2H), 4.41-4.43 (m, 2H), 7.37 (d, J=8.4 Hz, 1H), 7.60-7.62 (m, 2H), 7.82 (s, 1H), 7.89 (d, J=8.4 Hz, 1H), 7.97 (d, J=8.0 Hz, 1H).

Compound 198M: LC-MS (ESI) m/z: 365 [M+H₂O+Na]⁺. ¹H-NMR (DMSO-d₆, 400 MHz): δ (ppm) 7.50-7.56 (m, 2H), 7.76 (s, 1H), 7.79 (s, 1H), 8.22-8.25 (m, 2H).

Compound 198N: LC-MS (ESI) m/z: 326 [M+H]⁺.

Compound 1980: LC-MS (ESI) m/z: 508 [M+H]⁺.

Compound 198P: LC-MS (ESI) m/z: 660 [M−Boc+H]⁺.

Compound 198 was separated with chiral HPLC to give Compound 198-1 and Compound 198-2.

Compound 198-1: LC-MS (ESI) m/z: 660 [M+H]⁺; ¹H-NMR (DMSO-d₆, 400 MHz): δ (ppm) 0.75-1.35 (m, 8H), 1.50-1.77 (m, 7H), 2.56-2.78 (m, 2H), 2.82-3.05 (m, 1H), 3.48-3.52 (m, 2H), 3.80-4.15 (m, 2H), 4.97-5.03 (m, 1H), 6.73-6.76 (m, 2H), 7.40-7.57 (m, 4H), 7.78-7.81 (m, 1H), 7.98 (s, 1H), 8.18-8.28 (m, 2H). Chiral separation condition: MeOH contained 0.2% Methanol Ammonia; IC (4.6×150 mm, 5 μm); retention time: 7.26 minute.

Compound 198-2: LC-MS (ESI) m/z: 660 [M+H]⁺; ¹H-NMR (DMSO-d₆, 400 MHz): δ (ppm) 0.75-1.35 (m, 8H), 1.50-1.77 (m, 7H), 2.56-2.78 (m, 2H), 2.82-3.05 (m, 1H), 3.48-3.52 (m, 2H), 3.80-4.15 (m, 2H), 4.96-5.03 (m, 1H), 6.73-6.75 (m, 2H), 7.40-7.56 (m, 4H), 7.77-7.80 (m, 1H), 7.97 (s, 1H), 8.18-8.28 (m, 2H). Chiral separation condition: MeOH contained 0.2% Methanol Ammonia; IC (4.6×150 mm, 5 μm); retention time: 5.52 minute.

### Example 199

### Synthesis of N-(3-(3-amino-8-azabicyclo[3.2.1]octan-8-yl)-1-(4-bromophenyl)-1,1)-difluoro-3-oxopropan-2-yl)-6-(4-fluorophenyl)pyridine-3-sulfonamide trifluoroacetate (Compound 199)

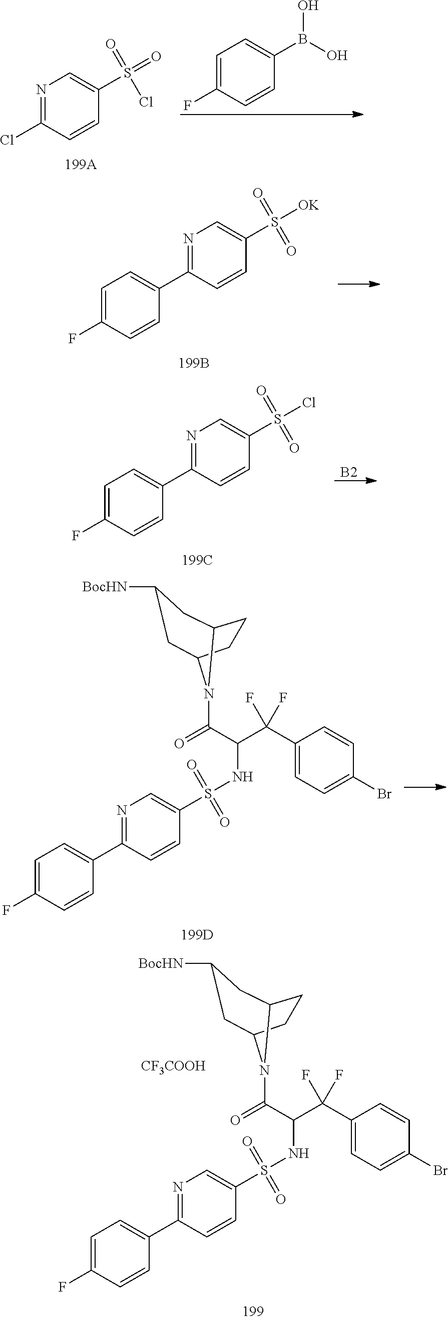

Compounds 199B, 199C, 199D, and 199 were synthesized by employing the procedures described for Compound 14C, Compound A1, Compounds 2D, and 1 using Compounds 199A, 199B, 199C, and 199D in lieu of Compound 14A, Compound A1-3, Compounds 2C, and 1A.

Compound 199B: LC-MS (ESI) m/z: 252[M−K]⁺. ¹H NMR (DMSO, 400 MHz): δ (ppm) 7.32 (t, J=9.2 Hz, 2H), 7.94 (d, J=8.0 Hz, 1H), 7.98-8.01 (m, 1H), 8.13-8.17 (m, 2H), 8.81 (d, J=1.6 Hz, 1H).

Compound 199C: LC-MS (ESI) m/z: 272[M+H]⁺.

Compound 199D: LC-MS (ESI) m/z: 723 [M+H]⁺.

Compound 199: LC-MS (ESI) m/z: 623 [M+H]⁺; ¹H-NMR (CD₃OD, 400 MHz): δ (ppm) 1.51-2.19 (m, 8H), 3.55-3.75 (m, 1H), 4.51-4.87 (m, 2H), 4.97-5.03 (m, 1H), 7.25-7.34 (m, 3H), 7.44-7.47 (m, 2H), 7.57 (d, J=8.4 Hz, 1H), 7.91-7.96 (m, 1H), 8.02-8.08 (m, 1H), 8.16-8.21 (m, 2H), 8.86 (s, 1H).

### Example 200

### Synthesis of (R)—N-(1-(4′-chloro-[1,1′-biphenyl]-4-yl)-3-(4-cyanopiperidin-1-yl)-1,1-difluoro-3-oxopropan-2-yl)-6-(cyclopentyloxy)naphthalene-2-sulfonamide (Compound 200-1), and (S)—N-(1-(4′-chloro-[1,1′-biphenyl]-4-yl)-3-(4-cyanopiperidin-1-yl)-1,1-difluoro-3-oxopropan-2-yl)-6-(cyclopentyloxy)naphthalene-2-sulfonamide (Compound 200-2)

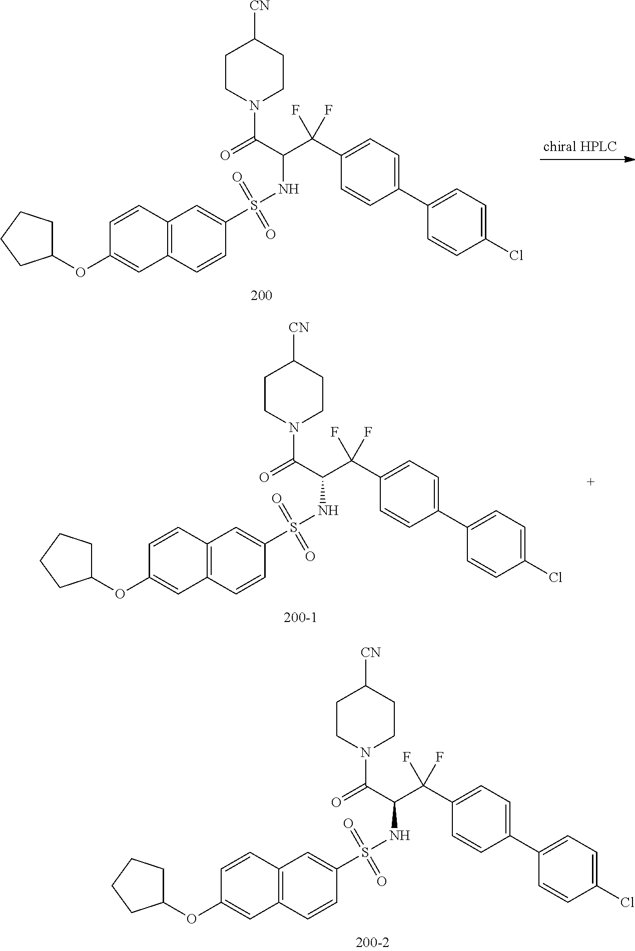 [SMILES: N#CC1CCN(C(=O)C(NS(=O)(=O)c2ccc3cc(OC4CCCC4)ccc3c2)C(F)(F)c2ccc(-c3ccc(Cl)cc3)cc2)CC1 ; N#CC1CCN(C(=O)[C@@H](NS(=O)(=O)c2ccc3cc(OC4CCCC4)ccc3c2)C(F)(F)c2ccc(-c3ccc(Cl)cc3)cc2)CC1 ; N#CC1CCN(C(=O)[C@H](NS(=O)(=O)c2ccc3cc(OC4CCCC4)ccc3c2)C(F)(F)c2ccc(-c3ccc(Cl)cc3)cc2)CC1]

Compound 200 of Example 200 corresponds to Compound 173b of Example 173. Compound 200 was separated with chiral HPLC to give Compound 200-1 and Compound 200-2.

Compound 200-1: LC-MS (ESI) m/z: 678 [M+H]⁺; ¹H-NMR (CDCl₃, 500 MHz): δ (ppm) 1.08-1.51 (m, 3H), 1.71-2.02 (m, 9H), 2.19-2.48 (m, 1H), 3.23-3.55 (m, 2H), 3.73-4.08 (m, 3H), 4.65-4.81 (m, 2H), 6.91-7.10 (m, 2H), 7.16-7.24 (m, 1H), 7.44-7.71 (m, 9H), 7.83-7.95 (m, 1H), 8.02-8.12 (m, 1H). Chiral separation condition: MeOH contained 0.2% Methanol Ammonia; (R,R)-Whelk-O1 (4.6×250 mm, 5 μm); retention time: 9.07 minute.

Compound 200-2: LC-MS (ESI) m/z: 678 [M+H]⁺; ¹H-NMR (CDCl₃, 500 MHz): δ (ppm) 1.08-1.23 (m, 3H), 1.24-1.68 (m, 3H), 1.83-1.98 (m, 6H), 2.56-2.68 (m, 1H), 3.17-3.50 (m, 4H), 4.77-4.87 (m, 2H), 5.95-5.97 (m, 1H), 7.02-7.09 (m, 1H), 7.15-7.21 (m, 1H), 7.42-7.49 (m, 8H), 7.56-7.60 (m, 1H), 7.65-7.72 (m, 2H), 8.15-8.16 (m, 1H). Chiral separation condition: MeOH contained 0.2% Methanol Ammonia; (R,R)-Whelk-O1 (4.6×250 mm, 5 μm); retention time: 7.28 minute.

### BIOLOGICAL EXAMPLES

The following describes ways in which the compounds described herein were tested to measure in vitro activity in enzymatic assays. A person of ordinary skill in the art would know that variations in the assay conditions could be used to determine the activity of the compounds.

### CGT Enzyme Assay

Full-length human CGT cDNA was cloned into the BamH1/Xho1 site of pcDNA3.1 (+) mammalian expression vector (V90-20, Invitrogen, Carlsbad, Calif.) and the plasmid was transfected into Chinese hamster ovary (CHO) cells. Lysate was prepared using M-PER (Mammalian Protein Extraction Reagent, ThermoFisher Scientific, Grand Island, N.Y.) in the presence of a protease inhibitor cocktail (P8340, Sigma, Saint Louis, Mo.). Each 100 mm dish (100% confluent, approximately 1×10⁷ cells) was lysed with 250 μL of M-PER containing protease inhibitors. Protein concentration was determined using a Pierce BCA protein assay kit (ThermoFisher Scientific). Four micrograms of CHO/CGT lysate was incubated with various concentrations of a compound (0.001 μM-50 μM) in 10 mM HEPES (pH 7.2) containing 35 μM dioleoylphosphatidylcholine, 5 mM MgCl₂, 5 mM MnCl₂, 1% BSA, 15 mM KCl, 1 mM EGTA, 8 mM CHAPS, 10 μM C6-NBD-dihydro-ceramide and 17.5 μM UDP-galactose in a final reaction volume of 20 μL at 37° C. for 1 hour. The final concentration of DMSO was 0.5% in both compound-treated and mock-treated samples. Tween 80 (0.01%) was optionally added to the reaction mixture to help solubilize the compounds. Each individual reaction was diluted with 80 μL of methanol:acetronitrile (1:3) containing 5 μM N-docdecanoyl-NBD-galactosylceramide (internal standard) to stop the reaction. 200 μL H₂O:acetonitile (1:1) was added to precipitate the protein. After sufficient mixing, the plates were centrifuged at 2469 g for 38 minutes, 200 μL of supernatant was transferred to a LC/MS 96-deep well plate and a second spin was performed at 2469 g for 10 minutes. The final supernatant was injected in triplicate for RapidFire/MS/MS analysis.

Quantitative Analysis Using the RapidFire/MS/MS Method:

The quantitative analysis of C6-NBD-dihydro-ceramide and C6-NBD-dihydro-galactosylceramide was performed on a Rapid Fire 360 high-throughput mass spectrometry system (Agilent Technologies, Palo Alto, Calif.) coupled with a API4000⁺ triple quadrupole mass spectrometer (Applied Biosystems, Concord, Ontario, Canada). RapidFire software packages, including RapidFire Control panel, RapidFire UI and RapidFire Integrator (Agilent Technologies), were used to control the RapidFire instrument and to process data. Analyst 1.6.2 software packages (Applied Biosystems) were used to control the MS system and acquire MS data. 10 L of sample was loaded on a micro-scale C4 solid-phase extraction (SPE) cartridge (Agilent Technologies) and salts were removed using water supplemented with 0.1% formic acid at the flow rate of 1.5 mL/min for 3 sec. C6-NBD-dihydro-ceramide and C6-NBD-dihydro-galactosylceramide were co-eluted into the mass spectrometer using acetonitrile containing 0.1% formic acid at the flow rate of 1.0 mL/min for 3 sec. The total cycle time of one injection was 8 secs. The MS/MS detection was performed in ESI negative mode. The mass transition of C6-NBD-dihydro-galactosylceramide was m/z 752.6→678.6 using a −40 V collision energy, the mass transition of C6-NBD-dihydro-ceramide was m/z 590.6→115.8 using a −40 V collision energy and the mass transition of N-docdecanoyl-NBD-galactosylceramide was m/z 820.9→746.3 using a −45 V collision energy.

The C6-NBD-dihydro-galactosylceramide reading was normalized first by dividing the peak area of C6-NBD-dihydro-galactosylceramide by the peak area of the internal standard, N-docdecanoyl-NBD-galactosylceramide. IC₅₀ values (see Table 1, where data ranges with one asterix (*) correspond to analysis using the RapidFire/MS/MS method, and data ranges with two asterixes (**) correspond to analysis using the RapidFire/MS/MS method with 0.01% Tween added) were generated from sigmoidal dose-response curves (variable slope) with GraphPad Prism software (GraphPad Software, Inc., San Diego, Calif.) using the normalized peak areas of C6-NBD-dihydro-galactosylceramide or the percent inhibition of C6-NBD-dihydro-galactosylceramide accumulation relative to DMSO control.

An alternative LC-MS/MS based method was also used. For the LC-MS/MS readout, the CGT assay was identical to the one described above except for the following: g of CHO/CGT lysate was used as the CGT enzyme source, C12:0 (2R—OH) ceramide was used as the ceramide substrate and 0.3 μM Cl₂ ceramide was used as the internal standard. Tween 80 was not present in the reaction mixture. The final supernatant was injected for LC-MS/MS analysis.

Quantitative analysis using the LC-MS/MS method: The quantitative analysis of C12:0 (2R—OH) ceramide and C12:0 (2R—OH) galactosylceramide by LC-MS/MS was performed on a Shimadzu ultra-fast liquid chromatography (Shimadzu, Japan) coupled with API 4000 mass spectrometer (Applied Biosystems, Concord, Ontario, Canada). Analyst 1.5 software packages (Applied Biosystems) were used to control the LC-MS/MS system, as well as for data acquisition and processing. 10 μL of sample was loaded onto a Luna C18 column (50 mm×2.0 mm, I.D. 3 μm, 100A) (Phenomenex, USA) for chromatographic separation. Mobile phase A consisted of HPLC grade water with 0.1% formic acid (v/v) and mobile phase B consisted of acetonitrile supplemented with 0.1% formic acid (v/v). The separation was achieved using the following gradient program at a flow rate of 0.8 mL/min: the initial mobile phase was 70% B, which was increased in a linear fashion to 95% B in 1.50 min, and then maintained at 95% B until 3.50 min. The mobile phase was then reset to 70% B within 0.01 min, and maintained until 4.50 min. The total run time was 4.50 min. The MS/MS detection was performed in ESI positive mode. The mass transition selected for quantification was m/z 498.5-264.3 for C12:0 (2R—OH) ceramide under the collision energy of 38.8 V, and the mass transition of C12:0 (2R—OH) galactosylceramide was m/z 660.6-264.5 under the collision energy of 51 V. The mass transition of Cl₂ ceramide which was used as internal standard was m/z 482.4-264.3 under the collision energy of 33V. Compound IC₅₀ values were calculated using a protocol identical to the one described for the RapidFire method (see Table 1, where data ranges without an asterix correspond to analysis using the LC-MS/MS method).

### CGT Cellular Assay

A stable cell line overexpressing human full-length CGT was made by transfecting Chinese hamster ovary (CHO) cells with 10 μg hCGT-pcDNA3.1(+) DNA and 30 μl of X-tremeGENE HP DNA transfection reagent (63/662,36001, Roche, Indianapolis, Ind.), followed by 800 μg/ml G418 selection. CHO cells stably expressing human full-length CGT (B5 cells) were seeded onto a CytoOne 96-well TC plate (USA Scientific, CC7682-7596) at 3×10⁴ cells/well in F12K media containing 10% FBS and incubated overnight at 37° C., 5% CO₂. The next day, the media was removed and replaced with 90 μl incubation media (F12K media containing 5% FBS and 1.1 μM eliglustat). In a separate 96-well tissue culture plate, the test compounds were serially diluted in DMSO (1 mM to 0.1 μM) followed by a 100-fold dilution with F12K media containing 5% FBS. 10 μL of compound was added to the cells and incubated at 37° C., 5% CO₂ for 2 hours. The final concentration of DMSO was 0.1% in both compound-treated and mock-treated cells. The C6-NBD-dihydro-ceramide substrate was diluted with F12K medium containing 5% FBS and 11% BSA to make a 110 μM substrate solution. 10 μL of substrate solution was added to the plate and incubated at 37° C., 5% CO₂ for 1 hour. Following the reaction, the plate was washed two times with PBS followed by the addition of 120 μL of lipid extraction solvent (methanol with 0.5% acetic acid) containing 1 μM of N-Dodecanoyl-NBD-galactosylceramide (internal standard). The sealed plate was placed on shaker for 2 hours (around 20 RPM) to extract product. After centrifugation at 2469 g for 30 mins, 80 μL of supernatant was transferred to a LC/MS 96-deep well plate containing 40 μL ddH₂O. After mixing thoroughly, a second spin was performed at 2469 g for 20 mins. The final supernatant was injected for LC-MS/MS analysis.

The quantitative analysis of C6-NBD-dihydro-ceramide and C6-NBD-dihydro-galactosylceramide was performed on a Shimadzu ultra-fast liquid chromatography (Shimadzu, Japan) coupled with API 4000 mass spectrometer (Applied Biosystems, Concord, Ontario, Canada). Analyst 1.5 software packages (Applied Biosystems) were used to control the LC-MS/MS system, as well as for data acquisition and processing. 10 μl of sample was loaded onto a KinetexC18 column (50 mm×2.1 mm, I.D. 2.6 μm, 100 Å) (Phenomenex, USA) for chromatographic separation. Mobile phase A consisted of HPLC grade water with 0.1% formic acid (v/v), and mobile phase B consisted of acetonitrile supplemented with 0.1% formic acid (v/v). The separation was achieved using the following gradient program at a flow rate of 0.7 mL/min: the initial mobile phase was 35% B and was ramped in a linear fashion to 98% B in 2.40 min. From 2.40 to 3.00 min, the gradient was maintained at 98% B. Then, mobile phase was reset to 35% B in 0.01 min, and maintained until 3.50 min. The total run time was 3.50 mins. The MS/MS detection was performed in ESI negative mode. The mass transition of C6-NBD-dihydro-ceramide was m/z 590.4→516.4 under the collision energy of −34 V. The mass transition of C6-NBD-dihydro-galactosylceramide was m/z 752.4→678.5 under the collision energy of −46 V. The mass transition of N-Dodecanoyl-NBD-galactosylceramide which was used as internal standard was m/z 820.5→115.6 under the collision energy of −72V. Compound IC₅₀ values were calculated using a protocol identical to the ones described above for the CGT enzyme assay.

Using the above CGT enzyme and CGT cellular assays, the compounds of Table 1 were tested. In Table 1, biological data range of IC₅₀ values calculated from the enzymatic assays are provided, where the ranges represented by A, B, C, or D without an asterisk correspond to analysis using the LC-MS/MS method; the ranges represented by A, B, C, or D with one asterix (*) correspond to analysis using the RapidFire method; and the ranges represented by A, B, C, or D with two asterix (**) correspond to analysis using the RapidFire/MS/MS method with 0.01% Tween added; and where:

A is <1 μM;

B is ≥1 to 10 μM;

C is >10 to 30 μM;

D is >30 to 100 μM; and

NT is not tested.

TABLE 1

| Example | | |
|---|---|---|
| No. | Name | IC₅₀ |
| 1-1 | N-((2R)-3-(3-amino-8-azabicyclo[3.2.1]octan-8-yl)-1-(4-bromophenyl)- | B*, B |
| | 1,1-difluoro-3-oxopropan-2-yl)-6-isopropoxynaphthalene-2-sulfonamide | |
| 1-2 | N-((2S)-3-(3-amino-8-azabicyclo[3.2.1]octan-8-yl)-1-(4-bromophenyl)- | C |
| | 1,1-difluoro-3-oxopropan-2-yl)-6-isopropoxynaphthalene-2-sulfonamide | |
| 2-2 | N-((2S)-3-(3-amino-8-azabicyclo[3.2.1]octan-8-yl)-1-(4-bromophenyl)- | D |
| | 1,1-difluoro-3-oxopropan-2-yl)-4-(2-ethylbutoxy)benzenesulfonamide | |
| 3 | N-(3-(3-amino-8-azabicyclo[3.2.1]octan-8-yl)-1-(4-bromophenyl)-1,1- | C |
| | difluoro-3-oxopropan-2-yl)-4′-fluoro-[1,1′-biphenyl]-4-sulfonamide | |
| 2-1 | N-((2R)-3-(3-amino-8-azabicyclo[3.2.1]octan-8-yl)-1-(4-bromophenyl)- | B |
| | 1,1-difluoro-3-oxopropan-2-yl)-4-(2-ethylbutoxy)benzenesulfonamide | |
| 4 | N-(3-(3-amino-8-azabicyclo[3.2.1]octan-8-yl)-1-(4-bromophenyl)-1,1- | C |
| | difluoro-3-oxopropan-2-yl)-2-(6-isopropoxynaphthalen-2- | |
| | yl)ethanesulfonamide | |
| 5 | N-(3-(3-amino-8-azabicyclo[3.2.1]octan-8-yl)-1-(4-bromophenyl)-1,1- | B |
| | difluoro-3-oxopropan-2-yl)-6-(cyclopentyloxy)naphthalene-2-sulfonamide | |
| 6-2 | N-((2S)-3-(3-amino-8-azabicyclo[3.2.1]octan-8-yl)-1-(4-bromophenyl)- | D |
| | 1,1-difluoro-3-oxopropan-2-yl)-6-butoxynaphthalene-2-sulfonamide | |
| 6-1 | N-((2R)-3-(3-amino-8-azabicyclo[3.2.1]octan-8-yl)-1-(4-bromophenyl)- | B |
| | 1,1-difluoro-3-oxopropan-2-yl)-6-butoxynaphthalene-2-sulfonamide | |
| 3-2 | N-((2S)-3-(3-amino-8-azabicyclo[3.2.1]octan-8-yl)-1-(4-bromophenyl)- | D |
| | 1,1-difluoro-3-oxopropan-2-yl)-4′-fluoro-[8 1,1′-biphenyl]-4-sulfonamide | |
| 7-2 | N-((2S)-3-(3-amino-8-azabicyclo[3.2.1]octan-8-yl)-1-(4-bromophenyl)- | D |
| | 1,1-difluoro-3-oxopropan-2-yl)-6-isobutoxynaphthalene-2-sulfonamide | |
| 7-1 | N-((2R)-3-(3-amino-8-azabicyclo[3.2.1]octan-8-yl)-1-(4-bromophenyl)- | B |
| | 1,1-difluoro-3-oxopropan-2-yl)-6-isobutoxynaphthalene-2-sulfonamide | |
| 8-2 | N-((2S)-3-(3-amino-8-azabicyclo[3.2.1]octan-8-yl)-1-(4-chlorophenyl)- | C |
| | 1,1-difluoro-3-oxopropan-2-yl)-6-isopropoxynaphthalene-2-sulfonamide | |
| 8-1 | N-((2R)-3-(3-amino-8-azabicyclo[3.2.1]octan-8-yl)-1-(4-chlorophenyl)- | B*, B |
| | 1,1-difluoro-3-oxopropan-2-yl)-6-isopropoxynaphthalene-2-sulfonamide | |
| 9-2 | N-((2S)-3-(3-amino-8-azabicyclo[3.2.1]octan-8-yl)-1-(4-bromophenyl)- | D |
| | 1,1-difluoro-3-oxopropan-2-yl)-2-(6-isopropoxynaphthalen-2-yl)-2- | |
| | oxoacetamide | |
| 9-1 | N-((2R)-3-(3-amino-8-azabicyclo[3.2.1]octan-8-yl)-1-(4-bromophenyl)- | C |
| | 1,1-difluoro-3-oxopropan-2-yl)-2-(6-isopropoxynaphthalen-2-yl)-2- | |
| | oxoacetamide | |
| 10-1 | N-((2R)-3-(3-amino-8-azabicyclo[3.2.1]octan-8-yl)-1-(4-bromophenyl)- | D |
| | 1,1-difluoro-3-oxopropan-2-yl)-6-(pyridin-2-yloxy)naphthalene-2- | |
| | sulfonamide | |
| 11-2 | N-((2S)-3-(3-amino-8-azabicyclo[3.2.1]octan-8-yl)-1-(4-bromophenyl)- | D |
| | 1,1-difluoro-3-oxopropan-2-yl)-4-(benzyloxy)benzenesulfonamide | |
| 12-1 | N-((2R)-3-(3-amino-8-azabicyclo[3.2.1]octan-8-yl)-1-(4-bromophenyl)- | C |
| | 1,1-difluoro-3-oxopropan-2-yl)-4- | |
| | (cyclohexylmethoxy)benzenesulfonamide | |
| 13-2 | N-((2S)-3-(3-amino-8-azabicyclo[3.2.1]octan-8-yl)-1-(4-bromophenyl)- | C |
| | 1,1-difluoro-3-oxopropan-2-yl)-6-((S)-sec-butoxy)naphthalene-2- | |
| | sulfonamide | |
| 13-1 | N-((2R)-3-(3-amino-8-azabicyclo[3.2.1]octan-8-yl)-1-(4-bromophenyl)- | B |
| | 1,1-difluoro-3-oxopropan-2-yl)-6-((R)-sec-butoxy)naphthalene-2- | |
| | sulfonamide | |
| 13-4 | N-((2S)-3-(3-amino-8-azabicyclo[3.2.1]octan-8-yl)-1-(4-bromophenyl)- | C |
| | 1,1-difluoro-3-oxopropan-2-yl)-6-((R)-sec-butoxy)naphthalene-2- | |
| | sulfonamide | |
| 13-3 | N-((2R)-3-(3-amino-8-azabicyclo[3.2.1]octan-8-yl)-1-(4-bromophenyl)- | B |
| | 1,1-difluoro-3-oxopropan-2-yl)-6-((S)-sec-butoxy)naphthalene-2- | |
| | sulfonamide | |
| 11-1 | N-((2R)-3-(3-amino-8-azabicyclo[3.2.1]octan-8-yl)-1-(4-bromophenyl)- | C |
| | 1,1-difluoro-3-oxopropan-2-yl)-4-(benzyloxy)benzenesulfonamide | |
| 199 | N-(3-(3-amino-8-azabicyclo[3.2.1]octan-8-yl)-1-(4-bromophenyl)-1,1- | D |
| | difluoro-3-oxopropan-2-yl)-6-(4-fluorophenyl)pyridine-3-sulfonamide | |
| 14 | N-(3-(3-amino-8-azabicyclo[3.2.1]octan-8-yl)-1-(4-bromophenyl)-1,1- | D |
| | difluoro-3-oxopropan-2-yl)-2-(5-(4-fluorophenyl)pyridin-2-yl)-2- | |
| | oxoacetamide | |
| 15-2 | N-((2S)-3-(3-amino-8-azabicyclo[3.2.1]octan-8-yl)-1-(4-bromophenyl)- | D |
| | 1,1-difluoro-3-oxopropan-2-yl)-4-((4- | |
| | chlorobenzyl)oxy)benzenesulfonamide | |
| 15-1 | N-((2R)-3-(3-amino-8-azabicyclo[3.2.1]octan-8-yl)-1-(4-bromophenyl)- | B |
| | 1,1-difluoro-3-oxopropan-2-yl)-4-((4- | |
| | chlorobenzyl)oxy)benzenesulfonamide | |
| 16-2 | N-((2S)-3-(3-amino-8-azabicyclo[3.2.1]octan-8-yl)-1-(4-bromophenyl)- | D |
| | 1,1-difluoro-3-oxopropan-2-yl)-6-isopropylnaphthalene-2-sulfonamide | |
| 16-1 | N-((2R)-3-(3-amino-8-azabicyclo[3.2.1]octan-8-yl)-1-(4-bromophenyl)- | B |
| | 1,1-difluoro-3-oxopropan-2-yl)-6-isopropylnaphthalene-2-sulfonamide | |
| 17-2 | N-((2S)-3-(3-amino-8-azabicyclo[3.2.1]octan-8-yl)-1-(4-bromophenyl)- | D |
| | 1,1-difluoro-3-oxopropan-2-yl)-3-((4- | |
| | fluorobenzyl)oxy)benzenesulfonamide | |
| 17-1 | N-((2R)-3-(3-amino-8-azabicyclo[3.2.1]octan-8-yl)-1-(4-bromophenyl)- | D |
| | 1,1-difluoro-3-oxopropan-2-yl)-3-((4- | |
| | fluorobenzyl)oxy)benzenesulfonamide | |
| 18-2 | N-((2S)-3-(3-amino-8-azabicyclo[3.2.1]octan-8-yl)-1-(4-bromophenyl)- | D |
| | 1,1-difluoro-3-oxopropan-2-yl)-4-((5-chloropyridin-2- | |
| | yl)oxy)benzenesulfonamide | |
| 18-1 | N-((2R)-3-(3-amino-8-azabicyclo[3.2.1]octan-8-yl)-1-(4-bromophenyl)- | C |
| | 1,1-difluoro-3-oxopropan-2-yl)-4-((5-chloropyridin-2- | |
| | yl)oxy)benzenesulfonamide | |
| 19-3 | (2S)-N-((2R)-3-(3-amino-8-azabicyclo[3.2.1]octan-8-yl)-1-(4- | B |
| | bromophenyl)-1,1-difluoro-3-oxopropan-2-yl)-2-hydroxy-2-(6- | |
| | isopropoxynaphthalen-2-yl)acetamide | |
| 19-4 | (2R)-N-((2S)-3-(3-amino-8-azabicyclo[3.2.1]octan-8-yl)-1-(4- | D*, D |
| | bromophenyl)-1,1-difluoro-3-oxopropan-2-yl)-2-hydroxy-2-(6- | |
| | isopropoxynaphthalen-2-yl)acetamide | |
| 20-2 | N-((2S)-3-(3-amino-8-azabicyclo[3.2.1]octan-8-yl)-1-(4-bromophenyl)- | C |
| | 1,1-difluoro-3-oxopropan-2-yl)-4- | |
| | (cyclopentylmethoxy)benzenesulfonamide | |
| 20-1 | N-((2R)-3-(3-amino-8-azabicyclo[3.2.1]octan-8-yl)-1-(4-bromophenyl)- | B |
| | 1,1-difluoro-3-oxopropan-2-yl)-4- | |
| | (cyclopentylmethoxy)benzenesulfonamide | |
| 21-1 | N-((2R)-1-(4-bromophenyl)-3-(3-(dimethylamino)-8- | C |
| | azabicyclo[3.2.1]octan-8-yl)-1,1-difluoro-3-oxopropan-2-yl)-6- | |
| | isopropoxynaphthalene-2-sulfonamide | |
| 21-2 | N-((2S)-1-(4-bromophenyl)-3-(3-(dimethylamino)-8- | D |
| | azabicyclo[3.2.1]octan-8-yl)-1,1-difluoro-3-oxopropan-2-yl)-6- | |
| | isopropoxynaphthalene-2-sulfonamide | |
| 19-5 | (2R)-N-((2R)-3-(3-amino-8-azabicyclo[3.2.1]octan-8-yl)-1-(4- | C |
| | bromophenyl)-1,1-difluoro-3-oxopropan-2-yl)-2-hydroxy-2-(6- | |
| | isopropoxynaphthalen-2-yl)acetamide | |
| 19-6 | (2S)-N-((2S)-3-(3-amino-8-azabicyclo[3.2.1]octan-8-yl)-1-(4- | D |
| | bromophenyl)-1,1-difluoro-3-oxopropan-2-yl)-2-hydroxy-2-(6- | |
| | isopropoxynaphthalen-2-yl)acetamide | |
| 22-2 | N-((2S)-3-(3-amino-8-azabicyclo[3.2.1]octan-8-yl)-1-(4-bromophenyl)- | D |
| | 1,1-difluoro-3-oxopropan-2-yl)-4-butoxybenzenesulfonamide | |
| 22-1 | N-((2R)-3-(3-amino-8-azabicyclo[3.2.1]octan-8-yl)-1-(4-bromophenyl)- | B |
| | 1,1-difluoro-3-oxopropan-2-yl)-4-butoxybenzenesulfonamide | |
| 23-2 | N-((2S)-3-(3-amino-8-azabicyclo[3.2.1]octan-8-yl)-1-(4-bromophenyl)- | D |
| | 1,1-difluoro-3-oxopropan-2-yl)-4-(hexyloxy)benzenesulfonamide | |
| 23-1 | N-((2R)-3-(3-amino-8-azabicyclo[3.2.1]octan-8-yl)-1-(4-bromophenyl)- | B |
| | 1,1-difluoro-3-oxopropan-2-yl)-4-(hexyloxy)benzenesulfonamide | |
| 24-2 | N-((2S)-3-(3-amino-8-azabicyclo[3.2.1]octan-8-yl)-1-(4-bromophenyl)- | D |
| | 1,1-difluoro-3-oxopropan-2-yl)-6-(benzyloxy)naphthalene-2-sulfonamide | |
| 24-1 | N-((2R)-3-(3-amino-8-azabicyclo[3.2.1]octan-8-yl)-1-(4-bromophenyl)- | B |
| | 1,1-difluoro-3-oxopropan-2-yl)-6-(benzyloxy)naphthalene-2-sulfonamide | |
| 25-2 | N-((2S)-3-(3-amino-8-azabicyclo[3.2.1]octan-8-yl)-1-(4-bromophenyl)- | D |
| | 1,1-difluoro-3-oxopropan-2-yl)-4-(4-fluorophenoxy)benzenesulfonamide | |
| 25-1 | N-((2R)-3-(3-amino-8-azabicyclo[3.2.1]octan-8-yl)-1-(4-bromophenyl)- | B |
| | 1,1-difluoro-3-oxopropan-2-yl)-4-(4-fluorophenoxy)benzenesulfonamide | |
| 26-2 | N-((2S)-3-(3-amino-8-azabicyclo[3.2.1]octan-8-yl)-1-(4-bromophenyl)- | D |
| | 1,1-difluoro-3-oxopropan-2-yl)-4-(2- | |
| | cyclohexylethoxy)benzenesulfonamide | |
| 26-1 | N-((2R)-3-(3-amino-8-azabicyclo[3.2.1]octan-8-yl)-1-(4-bromophenyl)- | B |
| | 1,1-difluoro-3-oxopropan-2-yl)-4-(2- | |
| | cyclohexylethoxy)benzenesulfonamide | |
| 5-2 | N-((2S)-3-(3-amino-8-azabicyclo[3.2.1]octan-8-yl)-1-(4-bromophenyl)- | B |
| | 1,1-difluoro-3-oxopropan-2-yl)-6-(cyclopentyloxy)naphthalene-2- | |
| | sulfonamide | |
| 5-1 | N-((2R)-3-(3-amino-8-azabicyclo[3.2.1]octan-8-yl)-1-(4-bromophenyl)- | A*, A**, A |
| | 1,1-difluoro-3-oxopropan-2-yl)-6-(cyclopentyloxy)naphthalene-2- | |
| | sulfonamide | |
| 27-2 | (S)-N-(3-(4-aminopiperidin-1-yl)-1-(4-bromophenyl)-1,1-difluoro-3- | C |
| | oxopropan-2-yl)-6-isopropoxynaphthalene-2-sulfonamide | |
| 27-1 | (R)-N-(3-(4-aminopiperidin-1-yl)-1-(4-bromophenyl)-1,1-difluoro-3- | B |
| | oxopropan-2-yl)-6-isopropoxynaphthalene-2-sulfonamide | |
| 28-1 | N-((2R)-3-(3-amino-8-azabicyclo[3.2.1]octan-8-yl)-1-(4-chlorophenyl)- | A* |
| | 1,1-difluoro-3-oxopropan-2-yl)-6-(cyclopentyloxy)naphthalene-2- | |
| | sulfonamide | |
| 28-2 | N-((2S)-3-(3-amino-8-azabicyclo[3.2.1]octan-8-yl)-1-(4-chlorophenyl)- | B* |
| | 1,1-difluoro-3-oxopropan-2-yl)-6-(cyclopentyloxy)naphthalene-2- | |
| | sulfonamide | |
| 29-1 | N-((2R)-3-(3-amino-8-azabicyclo[3.2.1]octan-8-yl)-1-(4-chlorophenyl)- | A* |
| | 1,1-difluoro-3-oxopropan-2-yl)-4- | |
| | (cyclohexylmethoxy)benzenesulfonamide | |
| 29-2 | N-((2S)-3-(3-amino-8-azabicyclo[3.2.1]octan-8-yl)-1-(4-chlorophenyl)- | B* |
| | 1,1-difluoro-3-oxopropan-2-yl)-4- | |
| | (cyclohexylmethoxy)benzenesulfonamide | |
| 30-2 | (S)-N-(3-(4-aminopiperidin-1-yl)-1-(4-chlorophenyl)-1,1-difluoro-3- | C* |
| | oxopropan-2-yl)-4-(cyclohexylmethoxy)benzenesulfonamide | |
| 30-1 | (R)-N-(3-(4-aminopiperidin-1-yl)-1-(4-chlorophenyl)-1,1-difluoro-3- | B* |
| | oxopropan-2-yl)-4-(cyclohexylmethoxy)benzenesulfonamide | |
| 31-1 | N-((2R)-1-([1,1′-biphenyl]-4-yl)-3-(3-amino-8-azabicyclo[3.2.1]octan-8- | B* |
| | yl)-1,1-difluoro-3-oxopropan-2-yl)-6-isopropoxynaphthalene-2- | |
| | sulfonamide | |
| 31-2 | N-((2S)-1-([1,1′-biphenyl]-4-yl)-3-(3-amino-8-azabicyclo[3.2.1]octan-8- | C* |
| | yl)-1,1-difluoro-3-oxopropan-2-yl)-6-isopropoxynaphthalene-2- | |
| | sulfonamide | |
| 32-2 | (S)-N-(3-(4-aminopiperidin-1-yl)-1-(4-bromophenyl)-1,1-difluoro-3- | C* |
| | oxopropan-2-yl)-6-(cyclopentyloxy)naphthalene-2-sulfonamide | |
| 32-1 | (R)-N-(3-(4-aminopiperidin-1-yl)-1-(4-bromophenyl)-1,1-difluoro-3- | A* |
| | oxopropan-2-yl)-6-(cyclopentyloxy)naphthalene-2-sulfonamide | |
| 33-2 | N-((2S)-3-(3-amino-8-azabicyclo[3.2.1]octan-8-yl)-1-(4-bromophenyl)- | D* |
| | 1,1-difluoro-3-oxopropan-2-yl)-6-(cyclohexyloxy)naphthalene-2- | |
| | sulfonamide | |
| 33-1 | N-((2R)-3-(3-amino-8-azabicyclo[3.2.1]octan-8-yl)-1-(4-bromophenyl)- | A* |
| | 1,1-difluoro-3-oxopropan-2-yl)-6-(cyclohexyloxy)naphthalene-2- | |
| | sulfonamide | |
| 34-2 | N-((2S)-3-(3-amino-8-azabicyclo[3.2.1]octan-8-yl)-1-(4-bromophenyl)- | D* |
| | 1,1-difluoro-3-oxopropan-2-yl)-6-(cyclopentyloxy)-N-methylnaphthalene- | |
| | 2-sulfonamide | |
| 34-1 | N-((2R)-3-(3-amino-8-azabicyclo[3.2.1]octan-8-yl)-1-(4-bromophenyl)- | A* |
| | 1,1-difluoro-3-oxopropan-2-yl)-6-(cyclopentyloxy)-N-methylnaphthalene- | |
| | 2-sulfonamide | |
| 35-2 | N-((2S)-1-([1,1′-biphenyl]-4-yl)-3-(3-amino-8-azabicyclo[3.2.1]octan-8- | C* |
| | yl)-1,1-difluoro-3-oxopropan-2-yl)-4- | |
| | (cyclohexylmethoxy)benzenesulfonamide | |
| 35-1 | N-((2R)-1-([1,1′-biphenyl]-4-yl)-3-(3-amino-8-azabicyclo[3.2.1]octan-8- | A* |
| | yl)-1,1-difluoro-3-oxopropan-2-yl)-4- | |
| | (cyclohexylmethoxy)benzenesulfonamide | |
| 36-1 | N-((2R)-1-([1,1′-biphenyl]-4-yl)-3-(3-amino-8-azabicyclo[3.2.1]octan-8- | A* |
| | yl)-1,1-difluoro-3-oxopropan-2-yl)-6-(cyclopentyloxy)naphthalene-2- | |
| | sulfonamide | |
| 36-2 | N-((2S)-1-([1,1′-biphenyl]-4-yl)-3-(3-amino-8-azabicyclo[3.2.1]octan-8- | B* |
| | yl)-1,1-difluoro-3-oxopropan-2-yl)-6-(cyclopentyloxy)naphthalene-2- | |
| | sulfonamide | |
| 37-2 | N-((2S)-3-(3-amino-8-azabicyclo[3.2.1]octan-8-yl)-1-(4-bromophenyl)- | D* |
| | 1,1-difluoro-3-oxopropan-2-yl)-6-(cyclohexyloxy)-N-methylnaphthalene- | |
| | 2-sulfonamide | |
| 37-1 | N-((2R)-3-(3-amino-8-azabicyclo[3.2.1]octan-8-yl)-1-(4-bromophenyl)- | A* |
| | 1,1-difluoro-3-oxopropan-2-yl)-6-(cyclohexyloxy)-N-methylnaphthalene- | |
| | 2-sulfonamide | |
| 38-2 | N-((2S)-3-(3-amino-8-azabicyclo[3.2.1]octan-8-yl)-1-(6- | B* |
| | bromonaphthalen-2-yl)-1,1-difluoro-3-oxopropan-2-yl)-6- | |
| | (cyclopentyloxy)naphthalene-2-sulfonamide | |
| 38-1 | N-((2R)-3-(3-amino-8-azabicyclo[3.2.1]octan-8-yl)-1-(6- | B* |
| | bromonaphthalen-2-yl)-1,1-difluoro-3-oxopropan-2-yl)-6- | |
| | (cyclopentyloxy)naphthalene-2-sulfonamide | |
| 39-2 | N-((2S)-3-(3-amino-8-azabicyclo[3.2.1]octan-8-yl)-1-(6- | C* |
| | bromonaphthalen-2-yl)-1,1-difluoro-3-oxopropan-2-yl)-4- | |
| | (cyclopentylmethoxy)benzenesulfonamide | |
| 39-1 | N-((2R)-3-(3-amino-8-azabicyclo[3.2.1]octan-8-yl)-1-(6- | B* |
| | bromonaphthalen-2-yl)-1,1-difluoro-3-oxopropan-2-yl)-4- | |
| | (cyclopentylmethoxy)benzenesulfonamide | |
| 40-2 | N-((2S)-3-(3-amino-8-azabicyclo[3.2.1]octan-8-yl)-1-(6- | C* |
| | bromonaphthalen-2-yl)-1,1-difluoro-3-oxopropan-2-yl)-4- | |
| | (cyclohexylmethoxy)benzenesulfonamide | |
| 40-1 | N-((2R)-3-(3-amino-8-azabicyclo[3.2.1]octan-8-yl)-1-(6- | B* |
| | bromonaphthalen-2-yl)-1,1-difluoro-3-oxopropan-2-yl)-4- | |
| | (cyclohexylmethoxy)benzenesulfonamide | |
| 41-3 | (2S)-N-((2R)-3-(3-amino-8-azabicyclo[3.2.1]octan-8-yl)-1-(4- | B* |
| | bromophenyl)-1,1-difluoro-3-oxopropan-2-yl)-2-(6- | |
| | (cyclopentyloxy)naphthalen-2-yl)-2-hydroxyacetamide | |
| 41-4 | (2R)-N-((2S)-3-(3-amino-8-azabicyclo[3.2.1]octan-8-yl)-1-(4- | D* |
| | bromophenyl)-1,1-difluoro-3-oxopropan-2-yl)-2-(6- | |
| | (cyclopentyloxy)naphthalen-2-yl)-2-hydroxyacetamide | |
| 41-5 | (2R)-N-((2R)-3-(3-amino-8-azabicyclo[3.2.1]octan-8-yl)-1-(4- | C* |
| | bromophenyl)-1,1-difluoro-3-oxopropan-2-yl)-2-(6- | |
| | (cyclopentyloxy)naphthalen-2-yl)-2-hydroxyacetamide | |
| 41-6 | (2S)-N-((2S)-3-(3-amino-8-azabicyclo[3.2.1]octan-8-yl)-1-(4- | D* |
| | bromophenyl)-1,1-difluoro-3-oxopropan-2-yl)-2-(6- | |
| | (cyclopentyloxy)naphthalen-2-yl)-2-hydroxyacetamide | |
| 42-1 | N-((2R)-3-(3-amino-8-azabicyclo[3.2.1]octan-8-yl)-1,1-difluoro-1-(4- | A* |
| | iodophenyl)-3-oxopropan-2-yl)-6-(cyclopentyloxy)naphthalene-2- | |
| | sulfonamide | |
| 43-1 | (R)-N-(1-([1,1′-biphenyl]-4-yl)-3-(4-aminopiperidin-1-yl)-1,1-difluoro-3- | A* |
| | oxopropan-2-yl)-6-(cyclopentyloxy)naphthalene-2-sulfonamide | |
| 43-2 | (S)-N-(1-([1,1′-biphenyl]-4-yl)-3-(4-aminopiperidin-1-yl)-1,1-difluoro-3- | B* |
| | oxopropan-2-yl)-6-(cyclopentyloxy)naphthalene-2-sulfonamide | |
| 44-1 | (R)-N-(3-(4-aminopiperidin-1-yl)-1-(4-bromophenyl)-1,1-difluoro-3- | A* |
| | oxopropan-2-yl)-6-(cyclopentyloxy)-N-methylnaphthalene-2-sulfonamide | |
| 44-2 | (S)-N-(3-(4-aminopiperidin-1-yl)-1-(4-bromophenyl)-1,1-difluoro-3- | B* |
| | oxopropan-2-yl)-6-(cyclopentyloxy)-N-methylnaphthalene-2-sulfonamide | |
| 45-1 | N-((2R)-3-(3-amino-8-azabicyclo[3.2.1]octan-8-yl)-1,1-difluoro-1-(4- | C* |
| | methoxyphenyl)-3-oxopropan-2-yl)-6-(cyclopentyloxy)naphthalene-2- | |
| | sulfonamide | |
| 45-2 | N-((2S)-3-(3-amino-8-azabicyclo[3.2.1]octan-8-yl)-1,1-difluoro-1-(4- | B* |
| | methoxyphenyl)-3-oxopropan-2-yl)-6-(cyclopentyloxy)naphthalene-2- | |
| | sulfonamide | |
| 46-1 | N-((2R)-3-(3-amino-8-azabicyclo[3.2.1]octan-8-yl)-1-(4-bromophenyl)- | B* |
| | 1,1-difluoro-3-oxopropan-2-yl)-6-isopropoxy-N-methylnaphthalene-2- | |
| | sulfonamide | |
| 46-2 | N-((2S)-3-(3-amino-8-azabicyclo[3.2.1]octan-8-yl)-1-(4-bromophenyl)- | D* |
| | 1,1-difluoro-3-oxopropan-2-yl)-6-isopropoxy-N-methylnaphthalene-2- | |
| | sulfonamide | |
| 47-1 | N-((2R)-3-(3-amino-8-azabicyclo[3.2.1]octan-8-yl)-1-(4-bromophenyl)- | B* |
| | 1,1-difluoro-3-oxopropan-2-yl)-6-cyclobutoxynaphthalene-2-sulfonamide | |
| 47-2 | N-((2S)-3-(3-amino-8-azabicyclo[3.2.1]octan-8-yl)-1-(4-bromophenyl)- | C* |
| | 1,1-difluoro-3-oxopropan-2-yl)-6-cyclobutoxynaphthalene-2-sulfonamide | |
| 48-1 | N-((2R)-3-(3-amino-8-azabicyclo[3.2.1]octan-8-yl)-1-(4′-chloro-[1,1′- | A*, A** |
| | biphenyl]-4-yl)-1,1-difluoro-3-oxopropan-2-yl)-6- | |
| | (cyclopentyloxy)naphthalene-2-sulfonamide | |
| 48-2 | N-((2S)-3-(3-amino-8-azabicyclo[3.2.1]octan-8-yl)-1-(4′-chloro-[1,1′- | B*, B** |
| | biphenyl]-4-yl)-1,1-difluoro-3-oxopropan-2-yl)-6- | |
| | (cyclopentyloxy)naphthalene-2-sulfonamide | |
| 49-2 | N-((2S)-1-([1,1′-biphenyl]-4-yl)-3-(3-amino-8-azabicyclo[3.2.1]octan-8- | C* |
| | yl)-1,1-difluoro-3-oxopropan-2-yl)-6-(cyclopentyloxy)-N- | |
| | methylnaphthalene-2-sulfonamide | |
| 49-1 | N-((2R)-1-([1,1′-biphenyl]-4-yl)-3-(3-amino-8-azabicyclo[3.2.1]octan-8- | B* |
| | yl)-1,1-difluoro-3-oxopropan-2-yl)-6-(cyclopentyloxy)-N- | |
| | methylnaphthalene-2-sulfonamide | |
| 50-1 | N-((2R)-3-(3-amino-8-azabicyclo[3.2.1]octan-8-yl)-1-(4′-chloro-[1,1′- | A* |
| | biphenyl]-4-yl)-1,1-difluoro-3-oxopropan-2-yl)-4- | |
| | (cyclohexylmethoxy)benzenesulfonamide | |
| 50-2 | N-((2S)-3-(3-amino-8-azabicyclo[3.2.1]octan-8-yl)-1-(4′-chloro-[1,1′- | C* |
| | biphenyl]-4-yl)-1,1-difluoro-3-oxopropan-2-yl)-4- | |
| | (cyclohexylmethoxy)benzenesulfonamide | |
| 51-1 | N-((2R)-3-(3-amino-8-azabicyclo[3.2.1]octan-8-yl)-1-(4-butoxyphenyl)- | B* |
| | 1,1-difluoro-3-oxopropan-2-yl)-6-(cyclopentyloxy)-N-methylnaphthalene- | |
| | 2-sulfonamide | |
| 51-2 | N-((2S)-3-(3-amino-8-azabicyclo[3.2.1]octan-8-yl)-1-(4-butoxyphenyl)- | B* |
| | 1,1-difluoro-3-oxopropan-2-yl)-6-(cyclopentyloxy)-N-methylnaphthalene- | |
| | 2-sulfonamide | |
| 52-2 | N-((2S)-3-(3-amino-8-azabicyclo[3.2.1]octan-8-yl)-1-(4-chlorophenyl)- | D* |
| | 1,1-difluoro-3-oxopropan-2-yl)-6-(cyclopentyloxy)-N-methylnaphthalene- | |
| | 2-sulfonamide | |
| 52-1 | N-((2R)-3-(3-amino-8-azabicyclo[3.2.1]octan-8-yl)-1-(4-chlorophenyl)- | A* |
| | 1,1-difluoro-3-oxopropan-2-yl)-6-(cyclopentyloxy)-N-methylnaphthalene- | |
| | 2-sulfonamide | |
| 53-1 | (R)-N-(3-(4-aminopiperidin-1-yl)-1-(4-butoxyphenyl)-1,1-difluoro-3- | B* |
| | oxopropan-2-yl)-6-(cyclopentyloxy)-N-methylnaphthalene-2-sulfonamide | |
| 53-2 | (S)-N-(3-(4-aminopiperidin-1-yl)-1-(4-butoxyphenyl)-1,1-difluoro-3- | C* |
| | oxopropan-2-yl)-6-(cyclopentyloxy)-N-methylnaphthalene-2-sulfonamide | |
| 54-2 | N-((2S)-3-(3-amino-8-azabicyclo[3.2.1]octan-8-yl)-1,1-difluoro-3-oxo-1- | C* |
| | (4-(trifluoromethyl)phenyl)propan-2-yl)-6-(cyclopentyloxy)naphthalene- | |
| | 2-sulfonamide | |
| 54-1 | N-((2R)-3-(3-amino-8-azabicyclo[3.2.1]octan-8-yl)-1,1-difluoro-3-oxo-1- | A* |
| | (4-(trifluoromethyl)phenyl)propan-2-yl)-6-(cyclopentyloxy)naphthalene- | |
| | 2-sulfonamide | |
| 55-1 | N-((2R)-3-(3-amino-8-azabicyclo[3.2.1]octan-8-yl)-1,1-difluoro-3-oxo-1- | A* |
| | (4′-(trifluoromethyl)-[1,1′-biphenyl]-4-yl)propan-2-yl)-6- | |
| | (cyclopentyloxy)naphthalene-2-sulfonamide | |
| 55-2 | N-((2S)-3-(3-amino-8-azabicyclo[3.2.1]octan-8-yl)-1,1-difluoro-3-oxo-1- | B* |
| | (4′-(trifluoromethyl)-[1,1′-biphenyl]-4-yl)propan-2-yl)-6- | |
| | (cyclopentyloxy)naphthalene-2-sulfonamide | |
| 56-2 | N-((2S)-3-(3-amino-8-azabicyclo[3.2.1]octan-8-yl)-1,1-difluoro-1-(4′- | B* |
| | fluoro-[1,1′-biphenyl]-4-yl)-3-oxopropan-2-yl)-6- | |
| | (cyclopentyloxy)naphthalene-2-sulfonamide | |
| 56-1 | N-((2R)-3-(3-amino-8-azabicyclo[3.2.1]octan-8-yl)-1,1-difluoro-1-(4′- | A* |
| | fluoro-[1,1′-biphenyl]-4-yl)-3-oxopropan-2-yl)-6- | |
| | (cyclopentyloxy)naphthalene-2-sulfonamide | |
| 57-2 | (S)-N-(3-(4-(aminomethyl)piperidin-1-yl)-1-(4-bromophenyl)-1,1- | C* |
| | difluoro-3-oxopropan-2-yl)-6-(cyclopentyloxy)naphthalene-2-sulfonamide | |
| 57-1 | (R)-N-(3-(4-(aminomethyl)piperidin-1-yl)-1-(4-bromophenyl)-1,1- | A* |
| | difluoro-3-oxopropan-2-yl)-6-(cyclopentyloxy)naphthalene-2-sulfonamide | |
| 58-1 | (R)-N-(3-(4-aminopiperidin-1-yl)-1-(4-butoxyphenyl)-1,1-difluoro-3- | B* |
| | oxopropan-2-yl)-4-(cyclohexylmethoxy)-N-methylbenzenesulfonamide | |
| 58-2 | (S)-N-(3-(4-aminopiperidin-1-yl)-1-(4-butoxyphenyl)-1,1-difluoro-3- | C* |
| | oxopropan-2-yl)-4-(cyclohexylmethoxy)-N-methylbenzenesulfonamide | |
| 59-1 | N-((2R)-3-(3-amino-8-azabicyclo[3.2.1]octan-8-yl)-1,1-difluoro-3-oxo-1- | A* |
| | (4-(trifluoromethyl)phenyl)propan-2-yl)-4- | |
| | (cyclohexylmethoxy)benzenesulfonamide | |
| 59-2 | N-((2S)-3-(3-amino-8-azabicyclo[3.2.1]octan-8-yl)-1,1-difluoro-3-oxo-1- | B* |
| | (4-(trifluoromethyl)phenyl)propan-2-yl)-4- | |
| | (cyclohexylmethoxy)benzenesulfonamide | |
| 60-1 | N-((2R)-3-(3-amino-8-azabicyclo[3.2.1]octan-8-yl)-1,1-difluoro-3-oxo-1- | B* |
| | (4-(trifluoromethyl)phenyl)propan-2-yl)-4- | |
| | (cyclopentylmethoxy)benzenesulfonamide | |
| 60-2 | N-((2S)-3-(3-amino-8-azabicyclo[3.2.1]octan-8-yl)-1,1-difluoro-3-oxo-1- | C* |
| | (4-(trifluoromethyl)phenyl)propan-2-yl)-4- | |
| | (cyclopentylmethoxy)benzenesulfonamide | |
| 62-2 | (S)-N-(3-(4-aminopiperidin-1-yl)-1,1-difluoro-3-oxo-1-(4- | B* |
| | phenoxyphenyl)propan-2-yl)-6-(cyclopentyloxy)-N-methylnaphthalene-2- | |
| | sulfonamide | |
| 62-1 | (R)-N-(3-(4-aminopiperidin-1-yl)-1,1-difluoro-3-oxo-1-(4- | B* |
| | phenoxyphenyl)propan-2-yl)-6-(cyclopentyloxy)-N-methylnaphthalene-2- | |
| | sulfonamide | |
| 63-2 | (S)-N-(3-(4-aminopiperidin-1-yl)-1,1-difluoro-3-oxo-1-(4- | C* |
| | phenoxyphenyl)propan-2-yl)-4-(cyclohexylmethoxy)benzenesulfonamide | |
| 63-1 | (R)-N-(3-(4-aminopiperidin-1-yl)-1,1-difluoro-3-oxo-1-(4- | B* |
| | phenoxyphenyl)propan-2-yl)-4-(cyclohexylmethoxy)benzenesulfonamide | |
| 64-2 | (S)-N-(3-(4-aminopiperidin-1-yl)-1,1-difluoro-3-oxo-1-(4- | C* |
| | phenoxyphenyl)propan-2-yl)-4-(cyclohexylmethoxy)-N- | |
| | methylbenzenesulfonamide | |
| 64-1 | (R)-N-(3-(4-aminopiperidin-1-yl)-1,1-difluoro-3-oxo-1-(4- | B* |
| | phenoxyphenyl)propan-2-yl)-4-(cyclohexylmethoxy)-N- | |
| | methylbenzenesulfonamide | |
| 66-2 | N-((2S)-1-([1,1′-biphenyl]-3-yl)-3-(3-amino-8-azabicyclo[3.2.1]octan-8- | B* |
| | yl)-1,1-difluoro-3-oxopropan-2-yl)-6-(cyclopentyloxy)naphthalene-2- | |
| | sulfonamide | |
| 66-1 | N-((2R)-1-([1,1′-biphenyl]-3-yl)-3-(3-amino-8-azabicyclo[3.2.1]octan-8- | A* |
| | yl)-1,1-difluoro-3-oxopropan-2-yl)-6-(cyclopentyloxy)naphthalene-2- | |
| | sulfonamide | |
| 65-2 | N-((2S)-3-(3-amino-8-azabicyclo[3.2.1]octan-8-yl)-1-(3-bromophenyl)- | C* |
| | 1,1-difluoro-3-oxopropan-2-yl)-6-(cyclopentyloxy)naphthalene-2- | |
| | sulfonamide | |
| 65-1 | N-((2R)-3-(3-amino-8-azabicyclo[3.2.1]octan-8-yl)-1-(3-bromophenyl)- | B* |
| | 1,1-difluoro-3-oxopropan-2-yl)-6-(cyclopentyloxy)naphthalene-2- | |
| | sulfonamide | |
| 67-2 | N-((2S)-3-(3-amino-8-azabicyclo[3.2.1]octan-8-yl)-1-(3-bromophenyl)- | D* |
| | 1,1-difluoro-3-oxopropan-2-yl)-6-(cyclopentyloxy)-N-methylnaphthalene- | |
| | 2-sulfonamide | |
| 67-1 | N-((2R)-3-(3-amino-8-azabicyclo[3.2.1]octan-8-yl)-1-(3-bromophenyl)- | B* |
| | 1,1-difluoro-3-oxopropan-2-yl)-6-(cyclopentyloxy)-N-methylnaphthalene- | |
| | 2-sulfonamide | |
| 68-2 | N-((2S)-3-(3-amino-8-azabicyclo[3.2.1]octan-8-yl)-1-(4-bromophenyl)- | D* |
| | 1,1-difluoro-3-oxopropan-2-yl)-6-(cyclopentyloxy)-N-ethylnaphthalene-2- | |
| | sulfonamide | |
| 68-1 | N-((2R)-3-(3-amino-8-azabicyclo[3.2.1]octan-8-yl)-1-(4-bromophenyl)- | B* |
| | 1,1-difluoro-3-oxopropan-2-yl)-6-(cyclopentyloxy)-N-ethylnaphthalene-2- | |
| | sulfonamide | |
| 61-1 | (R)-N-(3-(4-aminopiperidin-1-yl)-1,1-difluoro-3-oxo-1-(4- | A* |
| | phenoxyphenyl)propan-2-yl)-6-(cyclopentyloxy)naphthalene-2- | |
| | sulfonamide | |
| 61-2 | (S)-N-(3-(4-aminopiperidin-1-yl)-1,1-difluoro-3-oxo-1-(4- | A* |
| | phenoxyphenyl)propan-2-yl)-6-(cyclopentyloxy)naphthalene-2- | |
| | sulfonamide | |
| 69-1 | (R)-N-(1-([1,1′-biphenyl]-3-yl)-3-(4-aminopiperidin-1-yl)-1,1-difluoro-3- | B* |
| | oxopropan-2-yl)-6-(cyclopentyloxy)naphthalene-2-sulfonamide | |
| 69-2 | (S)-N-(1-([1,1′-biphenyl]-3-yl)-3-(4-aminopiperidin-1-yl)-1,1-difluoro-3- | D* |
| | oxopropan-2-yl)-6-(cyclopentyloxy)naphthalene-2-sulfonamide | |
| 70-2 | N-((2S)-1-([1,1′-biphenyl]-3-yl)-3-(3-amino-8-azabicyclo[3.2.1]octan-8- | B* |
| | yl)-1,1-difluoro-3-oxopropan-2-yl)-4- | |
| | (cyclohexylmethoxy)benzenesulfonamide | |
| 70-1 | N-((2R)-1-([1,1′-biphenyl]-3-yl)-3-(3-amino-8-azabicyclo[3.2.1]octan-8- | B* |
| | yl)-1,1-difluoro-3-oxopropan-2-yl)-4- | |
| | (cyclohexylmethoxy)benzenesulfonamide | |
| 75-1 | N-((2R)-3-(3-amino-8-azabicyclo[3.2.1]octan-8-yl)-1-(4-bromophenyl)- | B* |
| | 1,1-difluoro-3-oxopropan-2-yl)-1-(cyclopentylmethyl)-N-methyl-1H- | |
| | indazole-5-sulfonamide | |
| 75-2 | N-((2S)-3-(3-amino-8-azabicyclo[3.2.1]octan-8-yl)-1-(4-bromophenyl)- | D* |
| | 1,1-difluoro-3-oxopropan-2-yl)-1-(cyclopentylmethyl)-N-methyl-1H- | |
| | indazole-5-sulfonamide | |
| 71-1 | (R)-N-(3-(4-aminopiperidin-1-yl)-1,1-difluoro-3-oxo-1-(4- | A* |
| | (trifluoromethyl)phenyl)propan-2-yl)-6-(cyclopentyloxy)naphthalene-2- | |
| | sulfonamide | |
| 71-2 | (S)-N-(3-(4-aminopiperidin-1-yl)-1,1-difluoro-3-oxo-1-(4- | B* |
| | (trifluoromethyl)phenyl)propan-2-yl)-6-(cyclopentyloxy)naphthalene-2- | |
| | sulfonamide | |
| 72-2 | (S)-N-(3-(4-aminopiperidin-1-yl)-1,1-difluoro-3-oxo-1-(4- | D* |
| | (trifluoromethyl)phenyl)propan-2-yl)-4- | |
| | (cyclohexylmethoxy)benzenesulfonamide | |
| 72-1 | (R)-N-(3-(4-aminopiperidin-1-yl)-1,1-difluoro-3-oxo-1-(4- | B* |
| | (trifluoromethyl)phenyl)propan-2-yl)-4- | |
| | (cyclohexylmethoxy)benzenesulfonamide | |
| 73-1 | N-((2R)-3-(3-amino-8-azabicyclo[3.2.1]octan-8-yl)-1,1-difluoro-3-oxo-1- | A* |
| | (4-(trifluoromethyl)phenyl)propan-2-yl)-6-(cyclopentyloxy)-N- | |
| | methylnaphthalene-2-sulfonamide | |
| 73-2 | N-((2S)-3-(3-amino-8-azabicyclo[3.2.1]octan-8-yl)-1,1-difluoro-3-oxo-1- | B* |
| | (4-(trifluoromethyl)phenyl)propan-2-yl)-6-(cyclopentyloxy)-N- | |
| | methylnaphthalene-2-sulfonamide | |
| 74-2 | N-((2S)-3-(3-amino-8-azabicyclo[3.2.1]octan-8-yl)-1-(4-bromophenyl)- | D* |
| | 1,1-difluoro-3-oxopropan-2-yl)-1-(cyclopentylmethyl)-1H-indazole-5- | |
| | sulfonamide | |
| 74-1 | N-((2R)-3-(3-amino-8-azabicyclo[3.2.1]octan-8-yl)-1-(4-bromophenyl)- | B* |
| | 1,1-difluoro-3-oxopropan-2-yl)-1-(cyclopentylmethyl)-1H-indazole-5- | |
| | sulfonamide | |
| 42-2 | N-((2S)-3-(3-amino-8-azabicyclo[3.2.1]octan-8-yl)-1,1-difluoro-1-(4- | B* |
| | iodophenyl)-3-oxopropan-2-yl)-6-(cyclopentyloxy)naphthalene-2- | |
| | sulfonamide | |
| 76-2 | N-((2S)-3-(3-amino-8-azabicyclo[3.2.1]octan-8-yl)-1-(4-bromophenyl)- | D* |
| | 1,1-difluoro-3-oxopropan-2-yl)-6-(cyclopentyloxy)-N- | |
| | isopropylnaphthalene-2-sulfonamide | |
| 76-1 | N-((2R)-3-(3-amino-8-azabicyclo[3.2.1]octan-8-yl)-1-(4-bromophenyl)- | C* |
| | 1,1-difluoro-3-oxopropan-2-yl)-6-(cyclopentyloxy)-N- | |
| | isopropylnaphthalene-2-sulfonamide | |
| 77-2 | N-((2S)-3-(3-amino-8-azabicyclo[3.2.1]octan-8-yl)-1-(4-bromophenyl)- | D* |
| | 1,1-difluoro-3-oxopropan-2-yl)-2-(cyclopentylmethyl)-2H-indazole-5- | |
| | sulfonamide | |
| 77-1 | N-((2R)-3-(3-amino-8-azabicyclo[3.2.1]octan-8-yl)-1-(4-bromophenyl)- | B* |
| | 1,1-difluoro-3-oxopropan-2-yl)-2-(cyclopentylmethyl)-2H-indazole-5- | |
| | sulfonamide | |
| 78-2 | N-((2S)-3-(3-amino-8-azabicyclo[3.2.1]octan-8-yl)-1-(4-bromophenyl)- | D* |
| | 1,1-difluoro-3-oxopropan-2-yl)-2-(cyclopentylmethyl)-N-methyl-2H- | |
| | indazole-5-sulfonamide | |
| 78-1 | N-((2R)-3-(3-amino-8-azabicyclo[3.2.1]octan-8-yl)-1-(4-bromophenyl)- | B* |
| | 1,1-difluoro-3-oxopropan-2-yl)-2-(cyclopentylmethyl)-N-methyl-2H- | |
| | indazole-5-sulfonamide | |
| 79-1 | N-((2R)-3-(3-amino-8-azabicyclo[3.2.1]octan-8-yl)-1-(4-butoxyphenyl)- | B* |
| | 1,1-difluoro-3-oxopropan-2-yl)-4-(cyclohexylmethoxy)-N- | |
| | methylbenzenesulfonamide | |
| 79-2 | N-((2S)-3-(3-amino-8-azabicyclo[3.2.1]octan-8-yl)-1-(4-butoxyphenyl)- | B* |
| | 1,1-difluoro-3-oxopropan-2-yl)-4-(cyclohexylmethoxy)-N- | |
| | methylbenzenesulfonamide | |
| 80-2 | (S)-N-(3-(4-aminopiperidin-1-yl)-1-(4-butoxyphenyl)-1,1-difluoro-3- | B* |
| | oxopropan-2-yl)-6-(cyclopentyloxy)naphthalene-2-sulfonamide | |
| 80-1 | (R)-N-(3-(4-aminopiperidin-1-yl)-1-(4-butoxyphenyl)-1,1-difluoro-3- | A* |
| | oxopropan-2-yl)-6-(cyclopentyloxy)naphthalene-2-sulfonamide | |
| 81-2 | (S)-N-(3-(4-aminopiperidin-1-yl)-1-(4-butoxyphenyl)-1,1-difluoro-3- | D* |
| | oxopropan-2-yl)-4-(cyclohexylmethoxy)benzenesulfonamide | |
| 81-1 | (R)-N-(3-(4-aminopiperidin-1-yl)-1-(4-butoxyphenyl)-1,1-difluoro-3- | B* |
| | oxopropan-2-yl)-4-(cyclohexylmethoxy)benzenesulfonamide | |
| 82-2 | N-((2S)-3-(3-amino-8-azabicyclo[3.2.1]octan-8-yl)-1-(4-butoxyphenyl)- | B* |
| | 1,1-difluoro-3-oxopropan-2-yl)-6-(cyclopentyloxy)naphthalene-2- | |
| | sulfonamide | |
| 82-1 | N-((2R)-3-(3-amino-8-azabicyclo[3.2.1]octan-8-yl)-1-(4-butoxyphenyl)- | A* |
| | 1,1-difluoro-3-oxopropan-2-yl)-6-(cyclopentyloxy)naphthalene-2- | |
| | sulfonamide | |
| 83-2 | (S)-N-(3-(4-aminopiperidin-1-yl)-1-(3-butoxyphenyl)-1,1-difluoro-3- | B* |
| | oxopropan-2-yl)-6-(cyclopentyloxy)naphthalene-2-sulfonamide | |
| 83-1 | (R)-N-(3-(4-aminopiperidin-1-yl)-1-(3-butoxyphenyl)-1,1-difluoro-3- | A* |
| | oxopropan-2-yl)-6-(cyclopentyloxy)naphthalene-2-sulfonamide | |
| 85-1 | N-((2R)-3-(3-amino-8-azabicyclo[3.2.1]octan-8-yl)-1,1-difluoro-3-oxo-1- | B* |
| | (4-phenoxyphenyl)propan-2-yl)-4-(cyclohexylmethoxy)-N- | |
| | methylbenzenesulfonamide | |
| 85-2 | N-((2S)-3-(3-amino-8-azabicyclo[3.2.1]octan-8-yl)-1,1-difluoro-3-oxo-1- | C* |
| | (4-phenoxyphenyl)propan-2-yl)-4-(cyclohexylmethoxy)-N- | |
| | methylbenzenesulfonamide | |
| 84-2 | N-((2S)-3-(3-amino-8-azabicyclo[3.2.1]octan-8-yl)-1,1-difluoro-3-oxo-1- | C* |
| | (4-phenoxyphenyl)propan-2-yl)-4- | |
| | (cyclohexylmethoxy)benzenesulfonamide | |
| 84-1 | N-((2R)-3-(3-amino-8-azabicyclo[3.2.1]octan-8-yl)-1,1-difluoro-3-oxo-1- | B* |
| | (4-phenoxyphenyl)propan-2-yl)-4- | |
| | (cyclohexylmethoxy)benzenesulfonamide | |
| 87-2 | N-((2S)-3-(3-amino-8-azabicyclo[3.2.1]octan-8-yl)-1,1-difluoro-3-oxo-1- | B* |
| | (4-phenoxyphenyl)propan-2-yl)-6-(cyclopentyloxy)-N- | |
| | methylnaphthalene-2-sulfonamide | |
| 87-1 | N-((2R)-3-(3-amino-8-azabicyclo[3.2.1]octan-8-yl)-1,1-difluoro-3-oxo-1- | A* |
| | (4-phenoxyphenyl)propan-2-yl)-6-(cyclopentyloxy)-N- | |
| | methylnaphthalene-2-sulfonamide | |
| 86-2 | N-((2S)-3-(3-amino-8-azabicyclo[3.2.1]octan-8-yl)-1,1-difluoro-3-oxo-1- | B* |
| | (4-phenoxyphenyl)propan-2-yl)-6-(cyclopentyloxy)naphthalene-2- | |
| | sulfonamide | |
| 86-1 | N-((2R)-3-(3-amino-8-azabicyclo[3.2.1]octan-8-yl)-1,1-difluoro-3-oxo-1- | A* |
| | (4-phenoxyphenyl)propan-2-yl)-6-(cyclopentyloxy)naphthalene-2- | |
| | sulfonamide | |
| 88-2 | N-((2S)-3-(3-amino-8-azabicyclo[3.2.1]octan-8-yl)-1-(4-chlorophenyl)- | D* |
| | 1,1-difluoro-3-oxopropan-2-yl)-1-(cyclopentylmethyl)-1H-indazole-5- | |
| | sulfonamide | |
| 88-1 | N-((2R)-3-(3-amino-8-azabicyclo[3.2.1]octan-8-yl)-1-(4-chlorophenyl)- | B* |
| | 1,1-difluoro-3-oxopropan-2-yl)-1-(cyclopentylmethyl)-1H-indazole-5- | |
| | sulfonamide | |
| 89-2 | N-((2S)-3-(3-amino-8-azabicyclo[3.2.1]octan-8-yl)-1-(4-bromo-3- | B* |
| | methoxyphenyl)-1,1-difluoro-3-oxopropan-2-yl)-6- | |
| | (cyclopentyloxy)naphthalene-2-sulfonamide | |
| 89-1 | N-((2R)-3-(3-amino-8-azabicyclo[3.2.1]octan-8-yl)-1-(4-bromo-3- | A* |
| | methoxyphenyl)-1,1-difluoro-3-oxopropan-2-yl)-6- | |
| | (cyclopentyloxy)naphthalene-2-sulfonamide | |
| 90-1 | N-((2R)-3-(3-amino-8-azabicyclo[3.2.1]octan-8-yl)-1-(3-butoxyphenyl)- | A* |
| | 1,1-difluoro-3-oxopropan-2-yl)-6-(cyclopentyloxy)naphthalene-2- | |
| | sulfonamide | |
| 90-2 | N-((2S)-3-(3-amino-8-azabicyclo[3.2.1]octan-8-yl)-1-(3-butoxyphenyl)- | B* |
| | 1,1-difluoro-3-oxopropan-2-yl)-6-(cyclopentyloxy)naphthalene-2- | |
| | sulfonamide | |
| 91-1 | (R)-N-(3-(4-aminopiperidin-1-yl)-1-(4′-chloro-[1,1′-biphenyl]-4-yl)-1,1- | A*, A** |
| | difluoro-3-oxopropan-2-yl)-6-(cyclopentyloxy)naphthalene-2-sulfonamide | |
| 91-2 | (S)-N-(3-(4-aminopiperidin-1-yl)-1-(4′-chloro-[1,1′-biphenyl]-4-yl)-1,1- | B*, B** |
| | difluoro-3-oxopropan-2-yl)-6-(cyclopentyloxy)naphthalene-2-sulfonamide | |
| 92-1 | N-((2R)-3-(3-amino-8-azabicyclo[3.2.1]octan-8-yl)-1,1-difluoro-3-oxo-1- | A* |
| | (4-(trifluoromethyl)phenyl)propan-2-yl)-1-(cyclopentylmethyl)-1H- | |
| | indazole-5-sulfonamide | |
| 93-2 | (S)-N-(3-(4-aminopiperidin-1-yl)-1,1-difluoro-3-oxo-1-(4- | D* |
| | (trifluoromethyl)phenyl)propan-2-yl)-1-(cyclopentylmethyl)-1H-indazole- | |
| | 5-sulfonamide | |
| 93-1 | (R)-N-(3-(4-aminopiperidin-1-yl)-1,1-difluoro-3-oxo-1-(4- | B* |
| | (trifluoromethyl)phenyl)propan-2-yl)-1-(cyclopentylmethyl)-1H-indazole- | |
| | 5-sulfonamide | |
| 92-2 | N-((2S)-3-(3-amino-8-azabicyclo[3.2.1]octan-8-yl)-1,1-difluoro-3-oxo-1- | C* |
| | (4-(trifluoromethyl)phenyl)propan-2-yl)-1-(cyclopentylmethyl)-1H- | |
| | indazole-5-sulfonamide | |
| 94-2 | N-((2S)-3-(3-amino-8-azabicyclo[3.2.1]octan-8-yl)-1-(4′-chloro-[1,1′- | C* |
| | biphenyl]-4-yl)-1,1-difluoro-3-oxopropan-2-yl)-1-(cyclopentylmethyl)- | |
| | 1H-indazole-5-sulfonamide | |
| 94-1 | N-((2R)-3-(3-amino-8-azabicyclo[3.2.1]octan-8-yl)-1-(4′-chloro-[1,1′- | B* |
| | biphenyl]-4-yl)-1,1-difluoro-3-oxopropan-2-yl)-1-(cyclopentylmethyl)- | |
| | 1H-indazole-5-sulfonamide | |
| 95-2 | (S)-N-(3-(4-aminopiperidin-1-yl)-1-(4′-chloro-[1,1′-biphenyl]-4-yl)-1,1- | C* |
| | difluoro-3-oxopropan-2-yl)-1-(cyclopentylmethyl)-1H-indazole-5- | |
| | sulfonamide | |
| 95-1 | (R)-N-(3-(4-aminopiperidin-1-yl)-1-(4′-chloro-[1,1′-biphenyl]-4-yl)-1,1- | A* |
| | difluoro-3-oxopropan-2-yl)-1-(cyclopentylmethyl)-1H-indazole-5- | |
| | sulfonamide | |
| 96-2 | (S)-N-(3-(4-aminopiperidin-1-yl)-1-(4′-chloro-[1,1′-biphenyl]-4-yl)-1,1- | D*, D** |
| | difluoro-3-oxopropan-2-yl)-4-(cyclohexylmethoxy)benzenesulfonamide | |
| 96-1 | (R)-N-(3-(4-aminopiperidin-1-yl)-1-(4′-chloro-[1,1′-biphenyl]-4-yl)-1,1- | B*, B** |
| | difluoro-3-oxopropan-2-yl)-4-(cyclohexylmethoxy)benzenesulfonamide | |
| 97-1 | (R)-N-(3-(4-aminopiperidin-1-yl)-1-(4-(cyclohexyloxy)phenyl)-1,1- | B* |
| | difluoro-3-oxopropan-2-yl)-6-(cyclopentyloxy)naphthalene-2-sulfonamide | |
| 97-2 | (S)-N-(3-(4-aminopiperidin-1-yl)-1-(4-(cyclohexyloxy)phenyl)-1,1- | B* |
| | difluoro-3-oxopropan-2-yl)-6-(cyclopentyloxy)naphthalene-2-sulfonamide | |
| 98-2 | (S)-N-(3-(4-aminopiperidin-1-yl)-1-(4-chlorophenyl)-1,1-difluoro-3- | C* |
| | oxopropan-2-yl)-6-(cyclopentyloxy)naphthalene-2-sulfonamide | |
| 98-1 | (R)-N-(3-(4-aminopiperidin-1-yl)-1-(4-chlorophenyl)-1,1-difluoro-3- | B* |
| | oxopropan-2-yl)-6-(cyclopentyloxy)naphthalene-2-sulfonamide | |
| 99-2 | (S)-N-(3-(4-aminopiperidin-1-yl)-1-(4-chlorophenyl)-1,1-difluoro-3- | D* |
| | oxopropan-2-yl)-1-(cyclopentylmethyl)-1H-indazole-5-sulfonamide | |
| 99-1 | (R)-N-(3-(4-aminopiperidin-1-yl)-1-(4-chlorophenyl)-1,1-difluoro-3- | B* |
| | oxopropan-2-yl)-1-(cyclopentylmethyl)-1H-indazole-5-sulfonamide | |
| 100-2 | (S)-N-(3-(4-aminopiperidin-1-yl)-1-(4-chlorophenyl)-1,1-difluoro-3- | C* |
| | oxopropan-2-yl)-6-(cyclopentyloxy)-N-methylnaphthalene-2-sulfonamide | |
| 100-1 | (R)-N-(3-(4-aminopiperidin-1-yl)-1-(4-chlorophenyl)-1,1-difluoro-3- | A* |
| | oxopropan-2-yl)-6-(cyclopentyloxy)-N-methylnaphthalene-2-sulfonamide | |
| 101-1 | N-((2R)-3-(3-amino-8-azabicyclo[3.2.1]octan-8-yl)-1-(4-(5- | A* |
| | chlorothiophen-2-yl)phenyl)-1,1-difluoro-3-oxopropan-2-yl)-6- | |
| | (cyclopentyloxy)naphthalene-2-sulfonamide | |
| 101-2 | N-((2S)-3-(3-amino-8-azabicyclo[3.2.1]octan-8-yl)-1-(4-(5- | B* |
| | chlorothiophen-2-yl)phenyl)-1,1-difluoro-3-oxopropan-2-yl)-6- | |
| | (cyclopentyloxy)naphthalene-2-sulfonamide | |
| 102-2 | N-((2S)-3-(3-amino-8-azabicyclo[3.2.1]octan-8-yl)-1-(4- | B* |
| | (cyclohexyloxy)phenyl)-1,1-difluoro-3-oxopropan-2-yl)-6- | |
| | (cyclopentyloxy)naphthalene-2-sulfonamide | |
| 102-1 | N-((2R)-3-(3-amino-8-azabicyclo[3.2.1]octan-8-yl)-1-(4- | A* |
| | (cyclohexyloxy)phenyl)-1,1-difluoro-3-oxopropan-2-yl)-6- | |
| | (cyclopentyloxy)naphthalene-2-sulfonamide | |
| 103-2 | (S)-N-(3-(4-aminopiperidin-1-yl)-1-(4-(cyclohexyloxy)phenyl)-1,1- | D* |
| | difluoro-3-oxopropan-2-yl)-4-(cyclohexylmethoxy)benzenesulfonamide | |
| 103-1 | (R)-N-(3-(4-aminopiperidin-1-yl)-1-(4-(cyclohexyloxy)phenyl)-1,1- | A* |
| | difluoro-3-oxopropan-2-yl)-4-(cyclohexylmethoxy)benzenesulfonamide | |
| 104-2 | N-((2S)-3-(3-amino-8-azabicyclo[3.2.1]octan-8-yl)-1-(4-(5- | C* |
| | chlorothiophen-2-yl)phenyl)-1,1-difluoro-3-oxopropan-2-yl)-4- | |
| | (cyclohexylmethoxy)benzenesulfonamide | |
| 104-1 | N-((2R)-3-(3-amino-8-azabicyclo[3.2.1]octan-8-yl)-1-(4-(5- | B* |
| | chlorothiophen-2-yl)phenyl)-1,1-difluoro-3-oxopropan-2-yl)-4- | |
| | (cyclohexylmethoxy)benzenesulfonamide | |
| 105-2 | N-((2S)-3-(3-amino-8-azabicyclo[3.2.1]octan-8-yl)-1-(4- | C* |
| | (cyclohexyloxy)phenyl)-1,1-difluoro-3-oxopropan-2-yl)-4- | |
| | (cyclohexylmethoxy)benzenesulfonamide | |
| 105-1 | N-((2R)-3-(3-amino-8-azabicyclo[3.2.1]octan-8-yl)-1-(4- | A* |
| | (cyclohexyloxy)phenyl)-1,1-difluoro-3-oxopropan-2-yl)-4- | |
| | (cyclohexylmethoxy)benzenesulfonamide | |
| 106-2 | N-((2S)-3-(3-amino-8-azabicyclo[3.2.1]octan-8-yl)-1-(4- | B* |
| | (benzyloxy)phenyl)-1,1-difluoro-3-oxopropan-2-yl)-6-(cyclopentyloxy)- | |
| | N-methylnaphthalene-2-sulfonamide | |
| 106-1 | N-((2R)-3-(3-amino-8-azabicyclo[3.2.1]octan-8-yl)-1-(4- | B* |
| | (benzyloxy)phenyl)-1,1-difluoro-3-oxopropan-2-yl)-6-(cyclopentyloxy)- | |
| | N-methylnaphthalene-2-sulfonamide | |
| 107-2 | N-((2S)-3-(3-amino-8-azabicyclo[3.2.1]octan-8-yl)-1,1-difluoro-3-oxo-1- | B* |
| | (3-phenoxyphenyl)propan-2-yl)-6-(cyclopentyloxy)naphthalene-2- | |
| | sulfonamide | |
| 107-1 | N-((2R)-3-(3-amino-8-azabicyclo[3.2.1]octan-8-yl)-1,1-difluoro-3-oxo-1- | A* |
| | (3-phenoxyphenyl)propan-2-yl)-6-(cyclopentyloxy)naphthalene-2- | |
| | sulfonamide | |
| 109-2 | (S)-N-(3-(4-aminopiperidin-1-yl)-1,1-difluoro-3-oxo-1-(3- | B* |
| | phenoxyphenyl)propan-2-yl)-4-(cyclohexylmethoxy)benzenesulfonamide | |
| 109-1 | (R)-N-(3-(4-aminopiperidin-1-yl)-1,1-difluoro-3-oxo-1-(3- | B* |
| | phenoxyphenyl)propan-2-yl)-4-(cyclohexylmethoxy)benzenesulfonamide | |
| 108-2 | (S)-N-(3-(4-aminopiperidin-1-yl)-1,1-difluoro-3-oxo-1-(3- | B* |
| | phenoxyphenyl)propan-2-yl)-6-(cyclopentyloxy)naphthalene-2- | |
| | sulfonamide | |
| 108-1 | (R)-N-(3-(4-aminopiperidin-1-yl)-1,1-difluoro-3-oxo-1-(3- | A* |
| | phenoxyphenyl)propan-2-yl)-6-(cyclopentyloxy)naphthalene-2- | |
| | sulfonamide | |
| 110-2 | N-((2S)-3-(3-amino-8-azabicyclo[3.2.1]octan-8-yl)-1,1-difluoro-1-(4- | D* |
| | hydroxyphenyl)-3-oxopropan-2-yl)-6-(cyclopentyloxy)-N- | |
| | methylnaphthalene-2-sulfonamide | |
| 110-1 | N-((2R)-3-(3-amino-8-azabicyclo[3.2.1]octan-8-yl)-1,1-difluoro-1-(4- | B* |
| | hydroxyphenyl)-3-oxopropan-2-yl)-6-(cyclopentyloxy)-N- | |
| | methylnaphthalene-2-sulfonamide | |
| 111-2 | N-((2S)-3-(3-amino-8-azabicyclo[3.2.1]octan-8-yl)-1,1-difluoro-3-oxo-1- | C* |
| | (3-phenoxyphenyl)propan-2-yl)-6-(cyclopentyloxy)-N- | |
| | methylnaphthalene-2-sulfonamide | |
| 111-1 | N-((2R)-3-(3-amino-8-azabicyclo[3.2.1]octan-8-yl)-1,1-difluoro-3-oxo-1- | A* |
| | (3-phenoxyphenyl)propan-2-yl)-6-(cyclopentyloxy)-N- | |
| | methylnaphthalene-2-sulfonamide | |
| 112-2 | (S)-N-(3-(4-aminopiperidin-1-yl)-1-(4-(5-chlorothiophen-2-yl)phenyl)- | B* |
| | 1,1-difluoro-3-oxopropan-2-yl)-6-(cyclopentyloxy)naphthalene-2- | |
| | sulfonamide | |
| 112-1 | (R)-N-(3-(4-aminopiperidin-1-yl)-1-(4-(5-chlorothiophen-2-yl)phenyl)- | A* |
| | 1,1-difluoro-3-oxopropan-2-yl)-6-(cyclopentyloxy)naphthalene-2- | |
| | sulfonamide | |
| 113-2 | N-((2S)-3-(3-amino-8-azabicyclo[3.2.1]octan-8-yl)-1,1-difluoro-3-oxo-1- | B* |
| | (3-phenoxyphenyl)propan-2-yl)-4- | |
| | (cyclohexylmethoxy)benzenesulfonamide | |
| 113-1 | N-((2R)-3-(3-amino-8-azabicyclo[3.2.1]octan-8-yl)-1,1-difluoro-3-oxo-1- | A* |
| | (3-phenoxyphenyl)propan-2-yl)-4- | |
| | (cyclohexylmethoxy)benzenesulfonamide | |
| 114-2 | N-((2S)-3-(3-amino-8-azabicyclo[3.2.1]octan-8-yl)-1-(4-ethoxyphenyl)- | C* |
| | 1,1-difluoro-3-oxopropan-2-yl)-6-(cyclopentyloxy)-N-methylnaphthalene- | |
| | 2-sulfonamide | |
| 114-1 | N-((2R)-3-(3-amino-8-azabicyclo[3.2.1]octan-8-yl)-1-(4-ethoxyphenyl)- | B* |
| | 1,1-difluoro-3-oxopropan-2-yl)-6-(cyclopentyloxy)-N-methylnaphthalene- | |
| | 2-sulfonamide | |
| 115-2 | N-((2S)-3-(3-amino-8-azabicyclo[3.2.1]octan-8-yl)-1,1-difluoro-1-(4-(2- | C* |
| | morpholinoethoxy)phenyl)-3-oxopropan-2-yl)-6-(cyclopentyloxy)-N- | |
| | methylnaphthalene-2-sulfonamide | |
| 115-1 | N-((2R)-3-(3-amino-8-azabicyclo[3.2.1]octan-8-yl)-1,1-difluoro-1-(4-(2- | B* |
| | morpholinoethoxy)phenyl)-3-oxopropan-2-yl)-6-(cyclopentyloxy)-N- | |
| | methylnaphthalene-2-sulfonamide | |
| 116-2 | N-((2S)-3-(3-amino-8-azabicyclo[3.2.1]octan-8-yl)-1,1-difluoro-3-oxo-1- | D* |
| | (4-(2-(piperidin-1-yl)ethoxy)phenyl)propan-2-yl)-6-(cyclopentyloxy)-N- | |
| | methylnaphthalene-2-sulfonamide | |
| 116-1 | N-((2R)-3-(3-amino-8-azabicyclo[3.2.1]octan-8-yl)-1,1-difluoro-3-oxo-1- | D* |
| | (4-(2-(piperidin-1-yl)ethoxy)phenyl)propan-2-yl)-6-(cyclopentyloxy)-N- | |
| | methylnaphthalene-2-sulfonamide | |
| 117-1 | (R)-N-(3-(4-aminopiperidin-1-yl)-1-(4-(5-chlorothiophen-2-yl)phenyl)- | B* |
| | 1,1-difluoro-3-oxopropan-2-yl)-4- | |
| | (cyclohexylmethoxy)benzenesulfonamide | |
| 117-2 | (S)-N-(3-(4-aminopiperidin-1-yl)-1-(4-(5-chlorothiophen-2-yl)phenyl)- | C* |
| | 1,1-difluoro-3-oxopropan-2-yl)-4- | |
| | (cyclohexylmethoxy)benzenesulfonamide | |
| 118-1 | N-((2R)-3-(3-amino-8-azabicyclo[3.2.1]octan-8-yl)-1-(3-butoxy-4- | A* |
| | chlorophenyl)-1,1-difluoro-3-oxopropan-2-yl)-6- | |
| | (cyclopentyloxy)naphthalene-2-sulfonamide | |
| 118-2 | N-((2S)-3-(3-amino-8-azabicyclo[3.2.1]octan-8-yl)-1-(3-butoxy-4- | B* |
| | chlorophenyl)-1,1-difluoro-3-oxopropan-2-yl)-6- | |
| | (cyclopentyloxy)naphthalene-2-sulfonamide | |
| 119-2 | (S)-N-(3-(4-aminopiperidin-1-yl)-1-(4-bromophenyl)-1,1-difluoro-3- | C |
| | oxopropan-2-yl)-5-bromo-6-isopropoxynaphthalene-2-sulfonamide | |
| 119-1 | (R)-N-(3-(4-aminopiperidin-1-yl)-1-(4-bromophenyl)-1,1-difluoro-3- | B |
| | oxopropan-2-yl)-5-bromo-6-isopropoxynaphthalene-2-sulfonamide | |
| 120-1 | N-((2R)-3-(3-amino-8-azabicyclo[3.2.1]octan-8-yl)-1-(3-butoxy-4- | A* |
| | chlorophenyl)-1,1-difluoro-3-oxopropan-2-yl)-4- | |
| | (cyclohexylmethoxy)benzenesulfonamide | |
| 120-2 | N-((2S)-3-(3-amino-8-azabicyclo[3.2.1]octan-8-yl)-1-(3-butoxy-4- | B* |
| | chlorophenyl)-1,1-difluoro-3-oxopropan-2-yl)-4- | |
| | (cyclohexylmethoxy)benzenesulfonamide | |
| 121-1 | N-((2R)-3-(3-amino-8-azabicyclo[3.2.1]octan-8-yl)-1-(4-(4- | A* |
| | chlorophenoxy)phenyl)-1,1-difluoro-3-oxopropan-2-yl)-6- | |
| | (cyclopentyloxy)naphthalene-2-sulfonamide | |
| 121-2 | N-((2S)-3-(3-amino-8-azabicyclo[3.2.1]octan-8-yl)-1-(4-(4- | B* |
| | chlorophenoxy)phenyl)-1,1-difluoro-3-oxopropan-2-yl)-6- | |
| | (cyclopentyloxy)naphthalene-2-sulfonamide | |
| 122-2 | N-((S)-3-((R)-3-aminopiperidin-1-yl)-1-(4-bromophenyl)-1,1-difluoro-3- | D* |
| | oxopropan-2-yl)-6-(cyclopentyloxy)naphthalene-2-sulfonamide | |
| 122-1 | N-((R)-3-((R)-3-aminopiperidin-1-yl)-1-(4-bromophenyl)-1,1-difluoro-3- | B* |
| | oxopropan-2-yl)-6-(cyclopentyloxy)naphthalene-2-sulfonamide | |
| 123-2 | (S)-N-(3-(4-aminopiperidin-1-yl)-1-(4-chloro-3-methoxyphenyl)-1,1- | B* |
| | difluoro-3-oxopropan-2-yl)-6-(cyclopentyloxy)naphthalene-2-sulfonamide | |
| 123-1 | (R)-N-(3-(4-aminopiperidin-1-yl)-1-(4-chloro-3-methoxyphenyl)-1,1- | A* |
| | difluoro-3-oxopropan-2-yl)-6-(cyclopentyloxy)naphthalene-2-sulfonamide | |
| 124-2 | N-((2S)-3-(3-amino-8-azabicyclo[3.2.1]octan-8-yl)-1-(4-chloro-3- | B* |
| | methoxyphenyl)-1,1-difluoro-3-oxopropan-2-yl)-6- | |
| | (cyclopentyloxy)naphthalene-2-sulfonamide | |
| 124-1 | N-((2R)-3-(3-amino-8-azabicyclo[3.2.1]octan-8-yl)-1-(4-chloro-3- | A* |
| | methoxyphenyl)-1,1-difluoro-3-oxopropan-2-yl)-6- | |
| | (cyclopentyloxy)naphthalene-2-sulfonamide | |
| 125-2 | N-((2S)-3-(3-amino-8-azabicyclo[3.2.1]octan-8-yl)-1-(4-chloro-3- | C* |
| | methoxyphenyl)-1,1-difluoro-3-oxopropan-2-yl)-4- | |
| | (cyclohexylmethoxy)benzenesulfonamide | |
| 125-1 | N-((2R)-3-(3-amino-8-azabicyclo[3.2.1]octan-8-yl)-1-(4-chloro-3- | A* |
| | methoxyphenyl)-1,1-difluoro-3-oxopropan-2-yl)-4- | |
| | (cyclohexylmethoxy)benzenesulfonamide | |
| 127-2 | (S)-N-(3-(4-aminopiperidin-1-yl)-1-(3-butoxy-4-chlorophenyl)-1,1- | B* |
| | difluoro-3-oxopropan-2-yl)-4-(cyclohexylmethoxy)benzenesulfonamide | |
| 127-1 | (R)-N-(3-(4-aminopiperidin-1-yl)-1-(3-butoxy-4-chlorophenyl)-1,1- | A* |
| | difluoro-3-oxopropan-2-yl)-4-(cyclohexylmethoxy)benzenesulfonamide | |
| 126-2 | (S)-N-(3-(4-aminopiperidin-1-yl)-1-(3-butoxy-4-chlorophenyl)-1,1- | B* |
| | difluoro-3-oxopropan-2-yl)-6-(cyclopentyloxy)naphthalene-2-sulfonamide | |
| 126-1 | (R)-N-(3-(4-aminopiperidin-1-yl)-1-(3-butoxy-4-chlorophenyl)-1,1- | A* |
| | difluoro-3-oxopropan-2-yl)-6-(cyclopentyloxy)naphthalene-2-sulfonamide | |
| 128-2 | N-((2S)-3-(3-amino-8-azabicyclo[3.2.1]octan-8-yl)-1-(4-bromo-3- | B* |
| | methoxyphenyl)-1,1-difluoro-3-oxopropan-2-yl)-4- | |
| | (cyclohexylmethoxy)benzenesulfonamide | |
| 128-1 | N-((2R)-3-(3-amino-8-azabicyclo[3.2.1]octan-8-yl)-1-(4-bromo-3- | A* |
| | methoxyphenyl)-1,1-difluoro-3-oxopropan-2-yl)-4- | |
| | (cyclohexylmethoxy)benzenesulfonamide | |
| 129-2 | (S)-N-(3-(4-aminopiperidin-1-yl)-1-(4-chloro-3-methoxyphenyl)-1,1- | C* |
| | difluoro-3-oxopropan-2-yl)-4-(cyclohexylmethoxy)benzenesulfonamide | |
| 129-1 | (R)-N-(3-(4-aminopiperidin-1-yl)-1-(4-chloro-3-methoxyphenyl)-1,1- | B* |
| | difluoro-3-oxopropan-2-yl)-4-(cyclohexylmethoxy)benzenesulfonamide | |
| 130-2 | N-((2S)-3-(3-amino-8-azabicyclo[3.2.1]octan-8-yl)-1-(5-chlorothiophen-2- | C* |
| | yl)-1,1-difluoro-3-oxopropan-2-yl)-6-(cyclopentyloxy)naphthalene-2- | |
| | sulfonamide | |
| 130-1 | N-((2R)-3-(3-amino-8-azabicyclo[3.2.1]octan-8-yl)-1-(5-chlorothiophen- | B* |
| | 2-yl)-1,1-difluoro-3-oxopropan-2-yl)-6-(cyclopentyloxy)naphthalene-2- | |
| | sulfonamide | |
| 131-2 | (S)-N-(3-(4-aminopiperidin-1-yl)-1-(5-chlorothiophen-2-yl)-1,1-difluoro- | B* |
| | 3-oxopropan-2-yl)-6-(cyclopentyloxy)naphthalene-2-sulfonamide | |
| 131-1 | (R)-N-(3-(4-aminopiperidin-1-yl)-1-(5-chlorothiophen-2-yl)-1,1-difluoro- | B* |
| | 3-oxopropan-2-yl)-6-(cyclopentyloxy)naphthalene-2-sulfonamide | |
| 132-2 | N-((S)-3-((S)-3-aminopiperidin-1-yl)-1-(4-bromophenyl)-1,1-difluoro-3- | C* |
| | oxopropan-2-yl)-6-(cyclopentyloxy)naphthalene-2-sulfonamide | |
| 132-1 | N-((R)-3-((S)-3-aminopiperidin-1-yl)-1-(4-bromophenyl)-1,1-difluoro-3- | B* |
| | oxopropan-2-yl)-6-(cyclopentyloxy)naphthalene-2-sulfonamide | |
| 133-2 | N-((2S)-3-(3-amino-8-azabicyclo[3.2.1]octan-8-yl)-1-(4′-chloro-[1,1′- | D* |
| | biphenyl]-4-yl)-1,1-difluoro-3-oxopropan-2-yl)-2-(6- | |
| | (cyclopentyloxy)naphthalen-2-yl)-2-oxoacetamide | |
| 133-1 | N-((2R)-3-(3-amino-8-azabicyclo[3.2.1]octan-8-yl)-1-(4′-chloro-[1,1′- | B* |
| | biphenyl]-4-yl)-1,1-difluoro-3-oxopropan-2-yl)-2-(6- | |
| | (cyclopentyloxy)naphthalen-2-yl)-2-oxoacetamide | |
| 134-1 | (R)-N-(3-(4-aminopiperidin-1-yl)-1-(4′-chloro-[1,1′-biphenyl]-4-yl)-1,1- | C* |
| | difluoro-3-oxopropan-2-yl)-2-(4-(cyclohexylmethoxy)phenyl)-2- | |
| | oxoacetamide | |
| 134-2 | (S)-N-(3-(4-aminopiperidin-1-yl)-1-(4′-chloro-[1,1′-biphenyl]-4-yl)-1,1- | D* |
| | difluoro-3-oxopropan-2-yl)-2-(4-(cyclohexylmethoxy)phenyl)-2- | |
| | oxoacetamide | |
| 135-1 | N-((2R)-3-(3-amino-8-azabicyclo[3.2.1]octan-8-yl)-1-(4′-chloro-2- | A* |
| | methoxy-[1,1′-biphenyl]-4-yl)-1,1-difluoro-3-oxopropan-2-yl)-6- | |
| | (cyclopentyloxy)naphthalene-2-sulfonamide | |
| 135-2 | N-((2S)-3-(3-amino-8-azabicyclo[3.2.1]octan-8-yl)-1-(4′-chloro-2- | B* |
| | methoxy-[1,1′-biphenyl]-4-yl)-1,1-difluoro-3-oxopropan-2-yl)-6- | |
| | (cyclopentyloxy)naphthalene-2-sulfonamide | |
| 136-1 | (2R)-N-((2R)-3-(3-amino-8-azabicyclo[3.2.1]octan-8-yl)-1-(4′-chloro- | B* |
| | [1,1′-biphenyl]-4-yl)-1,1-difluoro-3-oxopropan-2-yl)-2-(6- | |
| | (cyclopentyloxy)naphthalen-2-yl)-2-hydroxyacetamide | |
| 136-2 | (2S)-N-((2S)-3-(3-amino-8-azabicyclo[3.2.1]octan-8-yl)-1-(4′-chloro-[1,1′- | D* |
| | biphenyl]-4-yl)-1,1-difluoro-3-oxopropan-2-yl)-2-(6- | |
| | (cyclopentyloxy)naphthalen-2-yl)-2-hydroxyacetamide | |
| 136-3 | (2S)-N-((2R)-3-(3-amino-8-azabicyclo[3.2.1]octan-8-yl)-1-(4′-chloro- | B*, B** |
| | [1,1′-biphenyl]-4-yl)-1,1-difluoro-3-oxopropan-2-yl)-2-(6- | |
| | (cyclopentyloxy)naphthalen-2-yl)-2-hydroxyacetamide | |
| 136-4 | (2R)-N-((2S)-3-(3-amino-8-azabicyclo[3.2.1]octan-8-yl)-1-(4′-chloro- | D* |
| | [1,1′-biphenyl]-4-yl)-1,1-difluoro-3-oxopropan-2-yl)-2-(6- | |
| | (cyclopentyloxy)naphthalen-2-yl)-2-hydroxyacetamide | |
| 137-2 | (S)-N-(1-(4′-chloro-[1,1′-biphenyl]-4-yl)-1,1-difluoro-3-oxo-3-(piperazin- | C* |
| | 1-yl)propan-2-yl)-6-(cyclopentyloxy)naphthalene-2-sulfonamide | |
| 137-1 | (R)-N-(1-(4′-chloro-[1,1′-biphenyl]-4-yl)-1,1-difluoro-3-oxo-3-(piperazin- | B* |
| | 1-yl)propan-2-yl)-6-(cyclopentyloxy)naphthalene-2-sulfonamide | |
| 138-2 | (S)-N-(3-(4-(aminomethyl)piperidin-1-yl)-1-(4′-chloro-[1,1′-biphenyl]-4- | B* |
| | yl)-1,1-difluoro-3-oxopropan-2-yl)-6-(cyclopentyloxy)naphthalene-2- | |
| | sulfonamide | |
| 138-1 | (R)-N-(3-(4-(aminomethyl)piperidin-1-yl)-1-(4′-chloro-[1,1′-biphenyl]-4- | A* |
| | yl)-1,1-difluoro-3-oxopropan-2-yl)-6-(cyclopentyloxy)naphthalene-2- | |
| | sulfonamide | |
| 139-2 | N-((S)-3-((R)-3-aminopyrrolidin-1-yl)-1-(4-bromophenyl)-1,1-difluoro-3- | B* |
| | oxopropan-2-yl)-6-(cyclopentyloxy)naphthalene-2-sulfonamide | |
| 139-1 | N-((R)-3-((R)-3-aminopyrrolidin-1-yl)-1-(4-bromophenyl)-1,1-difluoro-3- | A* |
| | oxopropan-2-yl)-6-(cyclopentyloxy)naphthalene-2-sulfonamide | |
| 140-2 | N-((S)-3-((S)-3-aminopyrrolidin-1-yl)-1-(4-bromophenyl)-1,1-difluoro-3- | C* |
| | oxopropan-2-yl)-6-(cyclopentyloxy)naphthalene-2-sulfonamide | |
| 140-1 | N-((R)-3-((S)-3-aminopyrrolidin-1-yl)-1-(4-bromophenyl)-1,1-difluoro-3- | B* |
| | oxopropan-2-yl)-6-(cyclopentyloxy)naphthalene-2-sulfonamide | |
| 141-2 | (S)-N-((S)-3-(4-aminopiperidin-1-yl)-1-(4′-chloro-[1,1′-biphenyl]-4-yl)- | C* |
| | 1,1-difluoro-3-oxopropan-2-yl)-2-(4-(cyclohexylmethoxy)phenyl)-2- | |
| | hydroxyacetamide | |
| 141-1 | (R)-N-((R)-3-(4-aminopiperidin-1-yl)-1-(4′-chloro-[1,1′-biphenyl]-4-yl)- | B* |
| | 1,1-difluoro-3-oxopropan-2-yl)-2-(4-(cyclohexylmethoxy)phenyl)-2- | |
| | hydroxyacetamide | |
| 141-3 | (S)-N-((R)-3-(4-aminopiperidin-1-yl)-1-(4′-chloro-[1,1′-biphenyl]-4-yl)- | B* |
| | 1,1-difluoro-3-oxopropan-2-yl)-2-(4-(cyclohexylmethoxy)phenyl)-2- | |
| | hydroxyacetamide | |
| 141-4 | (R)-N-((S)-3-(4-aminopiperidin-1-yl)-1-(4′-chloro-[1,1′-biphenyl]-4-yl)- | D* |
| | 1,1-difluoro-3-oxopropan-2-yl)-2-(4-(cyclohexylmethoxy)phenyl)-2- | |
| | hydroxyacetamide | |
| 142-2 | N-((2S)-3-(3-amino-8-azabicyclo[3.2.1]octan-8-yl)-1-(4′-chloro-2- | B** |
| | methoxy-[1,1′-biphenyl]-4-yl)-1,1-difluoro-3-oxopropan-2-yl)-4- | |
| | (cyclohexylmethoxy)benzenesulfonamide | |
| 142-1 | N-((2R)-3-(3-amino-8-azabicyclo[3.2.1]octan-8-yl)-1-(4′-chloro-2- | A** |
| | methoxy-[1,1′-biphenyl]-4-yl)-1,1-difluoro-3-oxopropan-2-yl)-4- | |
| | (cyclohexylmethoxy)benzenesulfonamide | |
| 143-1 | (R)-N-(3-(4-aminopiperidin-1-yl)-1-(4-bromo-3-methoxyphenyl)-1,1- | B** |
| | difluoro-3-oxopropan-2-yl)-6-(cyclopentyloxy)naphthalene-2-sulfonamide | |
| 143-2 | (S)-N-(3-(4-aminopiperidin-1-yl)-1-(4-bromo-3-methoxyphenyl)-1,1- | B** |
| | difluoro-3-oxopropan-2-yl)-6-(cyclopentyloxy)naphthalene-2-sulfonamide | |
| 144-1 | (R)-N-(3-(4-aminopiperidin-1-yl)-1-(4′-chloro-2-methoxy-[1,1′-biphenyl]- | A** |
| | 4-yl)-1,1-difluoro-3-oxopropan-2-yl)-6-(cyclopentyloxy)naphthalene-2- | |
| | sulfonamide | |
| 144-2 | (S)-N-(3-(4-aminopiperidin-1-yl)-1-(4′-chloro-2-methoxy-[1,1′-biphenyl]- | B** |
| | 4-yl)-1,1-difluoro-3-oxopropan-2-yl)-6-(cyclopentyloxy)naphthalene-2- | |
| | sulfonamide | |
| 145-2 | (S)-N-(3-(4-aminopiperidin-1-yl)-1-(4-bromo-3-methoxyphenyl)-1,1- | D** |
| | difluoro-3-oxopropan-2-yl)-4-(cyclohexylmethoxy)benzenesulfonamide | |
| 145-1 | (R)-N-(3-(4-aminopiperidin-1-yl)-1-(4-bromo-3-methoxyphenyl)-1,1- | A** |
| | difluoro-3-oxopropan-2-yl)-4-(cyclohexylmethoxy)benzenesulfonamide | |
| 146-1 | (R)-N-(3-(4-aminopiperidin-1-yl)-1-(4′-chloro-2-methoxy-[1,1′-biphenyl]- | A** |
| | 4-yl)-1,1-difluoro-3-oxopropan-2-yl)-4- | |
| | (cyclohexylmethoxy)benzenesulfonamide | |
| 146-2 | (S)-N-(3-(4-aminopiperidin-1-yl)-1-(4′-chloro-2-methoxy-[1,1′-biphenyl]- | B** |
| | 4-yl)-1,1-difluoro-3-oxopropan-2-yl)-4- | |
| | (cyclohexylmethoxy)benzenesulfonamide | |
| 147-2 | N-((S)-1-(4′-chloro-[1,1′-biphenyl]-4-yl)-1,1-difluoro-3-((3aR,6aS)- | C** |
| | hexahydropyrrolo[3,4-c]pyrrol-2(1H)-yl)-3-oxopropan-2-yl)-6- | |
| | (cyclopentyloxy)naphthalene-2-sulfonamide | |
| 147-1 | N-((R)-1-(4′-chloro-[1,1′-biphenyl]-4-yl)-1,1-difluoro-3-((3aR,6aS)- | A** |
| | hexahydropyrrolo[3,4-c]pyrrol-2(1H)-yl)-3-oxopropan-2-yl)-6- | |
| | (cyclopentyloxy)naphthalene-2-sulfonamide | |
| 148-1 | N-((R)-3-((S)-3-aminopyrrolidin-1-yl)-1-(4′-chloro-[1,1′-brphenyl]-4-yl)- | B** |
| | 1,1-difluoro-3-oxopropan-2-yl)-6-(cyclopentyloxy)naphthalene-2- | |
| | sulfonamide | |
| 148-2 | N-((S)-3-((S)-3-aminopyrrolidin-1-yl)-1-(4′-chloro-[1,1′-biphenyl]-4-yl)- | C** |
| | 1,1-difluoro-3-oxopropan-2-yl)-6-(cyclopentyloxy)naphthalene-2- | |
| | sulfonamide | |
| 200-2 | (S)-N-(1-(4′-chloro-[1,1′-biphenyl]-4-yl)-3-(4-cyanopiperidin-1-yl)-1,1- | NT |
| | difluoro-3-oxopropan-2-yl)-6-(cyclopentyloxy)naphthalene-2-sulfonamide | |
| 200-1 | (R)-N-(1-(4′-chloro-[1,1′-biphenyl]-4-yl)-3-(4-cyanopiperidin-1-yl)-1,1- | NT |
| | difluoro-3-oxopropan-2-yl)-6-(cyclopentyloxy)naphthalene-2-sulfonamide | |
| 149-1 | N-((R)-3-((R)-3-aminopyrrolidin-1-yl)-1-(4′-chloro-[1,1′-biphenyl]-4-yl)- | A** |
| | 1,1-difluoro-3-oxopropan-2-yl)-6-(cyclopentyloxy)naphthalene-2- | |
| | sulfonamide | |
| 149-2 | N-((S)-3-((R)-3-aminopyrrolidin-1-yl)-1-(4′-chloro-[1,1′-biphenyl]-4-yl)- | B** |
| | 1,1-difluoro-3-oxopropan-2-yl)-6-(cyclopentyloxy)naphthalene-2- | |
| | sulfonamide | |
| 150-1 | N-((R)-3-((1R,5S,8R)-8-amino-3-azabicyclo[3.2.1]octan-3-yl)-1-(4- | A** |
| | chlorophenyl)-1,1-difluoro-3-oxopropan-2-yl)-6- | |
| | (cyclopentyloxy)naphthalene-2-sulfonamide | |
| 150-2 | N-((S)-3-((1R,5S,8S)-8-amino-3-azabicyclo[3.2.1]octan-3-yl)-1-(4- | B** |
| | chlorophenyl)-1,1-difluoro-3-oxopropan-2-yl)-6- | |
| | (cyclopentyloxy)naphthalene-2-sulfonamide | |
| 151-2 | (S)-N-(1-(4′-chloro-[1,1′-biphenyl]-4-yl)-1,1-difluoro-3-oxo-3-(piperazin- | D** |
| | 1-yl)propan-2-yl)-4-(cyclohexylmethoxy)benzenesulfonamide | |
| 151-1 | (R)-N-(1-(4′-chloro-[1,1′-biphenyl]-4-yl)-1,1-difluoro-3-oxo-3-(piperazin- | B** |
| | 1-yl)propan-2-yl)-4-(cyclohexylmethoxy)benzenesulfonamide | |
| 152-2 | (2S)-N-((2S)-3-(3-amino-8-azabicyclo[3.2.1]octan-8-yl)-1-(4′-chloro-[1,1′- | D** |
| | biphenyl]-4-yl)-1,1-difluoro-3-oxopropan-2-yl)-2-(4- | |
| | (cyclohexylmethoxy)phenyl)-2-hydroxyacetamide | |
| 152-1 | (2R)-N-((2R)-3-(3-amino-8-azabicyclo[3.2.1]octan-8-yl)-1-(4′-chloro- | B** |
| | [1,1′-biphenyl]-4-yl)-1,1-difluoro-3-oxopropan-2-yl)-2-(4- | |
| | (cyclohexylmethoxy)phenyl)-2-hydroxyacetamide | |
| 153-2 | (S)-N-(3-(4-aminopiperidin-1-yl)-1-(4′-chloro-3′-ethoxy-[1,1′-biphenyl]-4- | D** |
| | yl)-1,1-difluoro-3-oxopropan-2-yl)-4- | |
| | (cyclohexylmethoxy)benzenesulfonamide | |
| 153-1 | (R)-N-(3-(4-aminopiperidin-1-yl)-1-(4′-chloro-3′-ethoxy-[1,1′-biphenyl]-4- | B** |
| | yl)-1,1-difluoro-3-oxopropan-2-yl)-4- | |
| | (cyclohexylmethoxy)benzenesulfonamide | |
| 154-1 | (R)-N-(3-(4-aminopiperidin-1-yl)-1-(4′-chloro-3′-ethoxy-[1,1′-biphenyl]-4- | B** |
| | yl)-1,1-difluoro-3-oxopropan-2-yl)-6-(cyclopentyloxy)naphthalene-2- | |
| | sulfonamide | |
| 154-2 | (S)-N-(3-(4-aminopiperidin-1-yl)-1-(4′-chloro-3′-ethoxy-[1,1′-biphenyl]-4- | B** |
| | yl)-1,1-difluoro-3-oxopropan-2-yl)-6-(cyclopentyloxy)naphthalene-2- | |
| | sulfonamide | |
| 152-3 | (2S)-N-((2R)-3-(3-amino-8-azabicyclo[3.2.1]octan-8-yl)-1-(4′-chloro- | B** |
| | [1,1′-biphenyl]-4-yl)-1,1-difluoro-3-oxopropan-2-yl)-2-(4- | |
| | (cyclohexylmethoxy)phenyl)-2-hydroxyacetamide | |
| 152-4 | (2R)-N-((2S)-3-(3-amino-8-azabicyclo[3.2.1]octan-8-yl)-1-(4′-chloro- | D** |
| | [1,1′-biphenyl]-4-yl)-1,1-difluoro-3-oxopropan-2-yl)-2-(4- | |
| | (cyclohexylmethoxy)phenyl)-2-hydroxyacetamide | |
| 155-1 | N-((R)-3-((R)-3-aminopyrrolidin-1-yl)-1-(4′-chloro-[1,1′-biphenyl]-4-yl)- | A** |
| | 1,1-difluoro-3-oxopropan-2-yl)-4- | |
| | (cyclohexylmethoxy)benzenesulfonamide | |
| 155-2 | N-((S)-3-((R)-3-aminopyrrolidin-1-yl)-1-(4′-chloro-[1,1′-biphenyl]-4-yl)- | B** |
| | 1,1-difluoro-3-oxopropan-2-yl)-4- | |
| | (cyclohexylmethoxy)benzenesulfonamide | |
| 156-2 | (S)-N-(1-([1,1′-biphenyl]-4-yl)-3-(4-aminopiperidin-1-yl)-1,1-difluoro-3- | D** |
| | oxopropan-2-yl)-4-(cyclohexylmethoxy)benzenesulfonamide | |
| 156-1 | (R)-N-(1-([1,1′-biphenyl]-4-yl)-3-(4-aminopiperidin-1-yl)-1,1-difluoro-3- | B** |
| | oxopropan-2-yl)-4-(cyclohexylmethoxy)benzenesulfonamide | |
| 157-1 | (R)-N-(3-aminopropyl)-3-(4′-chloro-[1,1′-biphenyl]-4-yl)-2-((6- | B** |
| | (cyclopentyloxy)naphthalene)-2-sulfonamido)-3,3-difluoro-N- | |
| | methylpropanamide | |
| 157-2 | (S)-N-(3-aminopropyl)-3-(4′-chloro-[1,1′-biphenyl]-4-yl)-2-((6- | D** |
| | (cyclopentyloxy)naphthalene)-2-sulfonamido)-3,3-difluoro-N- | |
| | methylpropanamide | |
| 158-1 | (2R)-N-((2R)-3-(3-amino-8-azabicyclo[3.2.1]octan-8-yl)-1-(6- | B** |
| | bromonaphthalen-2-yl)-1,1-difluoro-3-oxopropan-2-yl)-2-(6- | |
| | (cyclopentyloxy)naphthalen-2-yl)-2-hydroxyacetamide | |
| 158-2 | (2S)-N-((2S)-3-(3-amino-8-azabicyclo[3.2.1]octan-8-yl)-1-(6- | D** |
| | bromonaphthalen-2-yl)-1,1-difluoro-3-oxopropan-2-yl)-2-(6- | |
| | (cyclopentyloxy)naphthalen-2-yl)-2-hydroxyacetamide | |
| 158-3 | (2S)-N-((2R)-3-(3-amino-8-azabicyclo[3.2.1]octan-8-yl)-1-(6- | C** |
| | bromonaphthalen-2-yl)-1,1-difluoro-3-oxopropan-2-yl)-2-(6- | |
| | (cyclopentyloxy)naphthalen-2-yl)-2-hydroxyacetamide | |
| 158-4 | (2R)-N-((2S)-3-(3-amino-8-azabicyclo[3.2.1]octan-8-yl)-1-(6- | D** |
| | bromonaphthalen-2-yl)-1,1-difluoro-3-oxopropan-2-yl)-2-(6- | |
| | (cyclopentyloxy)naphthalen-2-yl)-2-hydroxyacetamide | |
| 159-3 | (S)-N-((R)-3-(4-aminopiperidin-1-yl)-1-(4′-chloro-[1,1′-biphenyl]-4-yl)- | B** |
| | 1,1-difluoro-3-oxopropan-2-yl)-2-(6-(cyclopentyloxy)naphthalen-2-yl)-2- | |
| | hydroxyacetamide | |
| 159-4 | (R)-N-((S)-3-(4-aminopiperidin-1-yl)-1-(4′-chloro-[1,1′-biphenyl]-4-yl)- | D** |
| | 1,1-difluoro-3-oxopropan-2-yl)-2-(6-(cyclopentyloxy)naphthalen-2-yl)-2- | |
| | hydroxyacetamide | |
| 160-2 | N-((2S)-3-(3-amino-8-azabicyclo[3.2.1]octan-8-yl)-1-(6- | D** |
| | bromonaphthalen-2-yl)-1,1-difluoro-3-oxopropan-2-yl)-2-(6- | |
| | (cyclopentyloxy)naphthalen-2-yl)-2-oxoacetamide | |
| 160-1 | N-((2R)-3-(3-amino-8-azabicyclo[3.2.1]octan-8-yl)-1-(6- | C** |
| | bromonaphthalen-2-yl)-1,1-difluoro-3-oxopropan-2-yl)-2-(6- | |
| | (cyclopentyloxy)naphthalen-2-yl)-2-oxoacetamide | |
| 161-1 | N-((R)-3-((R)-4-amino-3,3-difluoropiperidin-1-yl)-1-(4′-chloro-[1,1′- | B** |
| | biphenyl]-4-yl)-1,1-difluoro-3-oxopropan-2-yl)-6- | |
| | (cyclopentyloxy)naphthalene-2-sulfonamide | |
| 161-2 | N-((S)-3-((S)-4-amino-3,3-difluoropiperidin-1-yl)-1-(4′-chloro-[1,1′- | D** |
| | biphenyl]-4-yl)-1,1-difluoro-3-oxopropan-2-yl)-6- | |
| | (cyclopentyloxy)naphthalene-2-sulfonamide | |
| 162-1 | (R)-N-(3-(4-aminopiperidin-1-yl)-1-(5-(4-chlorophenyl)pyridin-2-yl)-1,1- | B** |
| | difluoro-3-oxopropan-2-yl)-6-(cyclopentyloxy)naphthalene-2-sulfonamide | |
| 162-2 | (S)-N-(3-(4-aminopiperidin-1-yl)-1-(5-(4-chlorophenyl)pyridin-2-yl)-1,1- | C** |
| | difluoro-3-oxopropan-2-yl)-6-(cyclopentyloxy)naphthalene-2-sulfonamide | |
| 163-1 | (R)-N-(3-(4-aminopiperidin-1-yl)-1-(5-(4-chlorophenyl)pyridin-2-yl)-1,1- | B** |
| | difluoro-3-oxopropan-2-yl)-4-(cyclohexylmethoxy)benzenesulfonamide | |
| 163-2 | (S)-N-(3-(4-aminopiperidin-1-yl)-1-(5-(4-chlorophenyl)pyridin-2-yl)-1,1- | D** |
| | difluoro-3-oxopropan-2-yl)-4-(cyclohexylmethoxy)benzenesulfonamide | |
| 159-1 | (R)-N-((R)-3-(4-aminopiperidin-1-yl)-1-(4′-chloro-[1,1′-biphenyl]-4-yl)- | B** |
| | 1,1-difluoro-3-oxopropan-2-yl)-2-(6-(cyclopentyloxy)naphthalen-2-yl)-2- | |
| | hydroxyacetamide | |
| 159-2 | (S)-N-((S)-3-(4-aminopiperidin-1-yl)-1-(4′-chloro-[1,1′-biphenyl]-4-yl)- | D** |
| | 1,1-difluoro-3-oxopropan-2-yl)-2-(6-(cyclopentyloxy)naphthalen-2-yl)-2- | |
| | hydroxyacetamide | |
| 164-2 | (S)-N-(3-(4-aminopiperidin-1-yl)-1-(4′-chloro-[1,1′-biphenyl]-4-yl)-1,1- | B** |
| | difluoro-3-oxopropan-2-yl)-6-(cyclopentyloxy)benzo[b]thiophene-2- | |
| | sulfonamide | |
| 164-1 | (R)-N-(3-(4-aminopiperidin-1-yl)-1-(4′-chloro-[1,1′-biphenyl]-4-yl)-1,1- | B** |
| | difluoro-3-oxopropan-2-yl)-6-(cyclopentyloxy)benzo[b]thiophene-2- | |
| | sulfonamide | |
| 165-1 | (S)-N-(3-(4-aminopiperidin-1-yl)-1-(4′-chloro-[1,1′-biphenyl]-4-yl)-1,1- | D** |
| | difluoro-3-oxopropan-2-yl)-5-((cyclohexyloxy)methyl)thiophene-2- | |
| | sulfonamide | |
| 165-2 | (R)-N-(3-(4-aminopiperidin-1-yl)-1-(4′-chloro-[1,1′-biphenyl]-4-yl)-1,1- | D** |
| | difluoro-3-oxopropan-2-yl)-5-((cyclohexyloxy)methyl)thiophene-2- | |
| | sulfonamide | |
| 166-1 | (R)-N-(3-(4-aminopiperidin-1-yl)-1,1-difluoro-3-oxo-1-(4-(3,3,3- | B** |
| | trifluoroprop-1-en-2-yl)phenyl)propan-2-yl)-6- | |
| | (cyclopentyloxy)naphthalene-2-sulfonamide | |
| 166-2 | (S)-N-(3-(4-aminopiperidin-1-yl)-1,1-difluoro-3-oxo-1-(4-(3,3,3- | B** |
| | trifluoroprop-1-en-2-yl)phenyl)propan-2-yl)-6- | |
| | (cyclopentyloxy)naphthalene-2-sulfonamide | |
| 167-2 | N-((2S)-3-(3-amino-8-azabicyclo[3.2.1]octan-8-yl)-1-(4-bromophenyl)- | B** |
| | 1,1-difluoro-3-oxopropan-2-yl)-6-(cyclopentyloxy)benzo[b]thiophene-2- | |
| | sulfonamide | |
| 167-1 | N-((2R)-3-(3-amino-8-azabicyclo[3.2.1]octan-8-yl)-1-(4-bromophenyl)- | B** |
| | 1,1-difluoro-3-oxopropan-2-yl)-6-(cyclopentyloxy)benzo[b]thiophene-2- | |
| | sulfonamide | |
| 168-1 | (R)-N-(3-(4-aminopiperidin-1-yl)-1-(4-chloro-3-cyclopropoxyphenyl)-1,1- | B** |
| | difluoro-3-oxopropan-2-yl)-6-(cyclopentyloxy)naphthalene-2-sulfonamide | |
| 168-2 | (S)-N-(3-(4-aminopiperidin-1-yl)-1-(4-chloro-3-cyclopropoxyphenyl)-1,1- | C** |
| | difluoro-3-oxopropan-2-yl)-6-(cyclopentyloxy)naphthalene-2-sulfonamide | |
| 169-2 | (S)-N-(3-(4-aminopiperidin-1-yl)-1-(4-chloro-3-propoxyphenyl)-1,1- | B** |
| | difluoro-3-oxopropan-2-yl)-6-(cyclopentyloxy)naphthalene-2-sulfonamide | |
| 169-1 | (R)-N-(3-(4-aminopiperidin-1-yl)-1-(4-chloro-3-propoxyphenyl)-1,1- | A** |
| | difluoro-3-oxopropan-2-yl)-6-(cyclopentyloxy)naphthalene-2-sulfonamide | |
| 170-2 | (S)-N-(3-(4-aminopiperidin-1-yl)-1-(4-chloro-3-(cyclopentyloxy)phenyl)- | C** |
| | 1,1-difluoro-3-oxopropan-2-yl)-6-(cyclopentyloxy)naphthalene-2- | |
| | sulfonamide | |
| 170-1 | (R)-N-(3-(4-aminopiperidin-1-yl)-1-(4-chloro-3-(cyclopentyloxy)phenyl)- | B** |
| | 1,1-difluoro-3-oxopropan-2-yl)-6-(cyclopentyloxy)naphthalene-2- | |
| | sulfonamide | |
| 171-2 | N-((S)-3-((S)-3-aminopyrrolidin-1-yl)-1-(4′-chloro-[1,1′-biphenyl]-4-yl)- | D** |
| | 1,1-difluoro-3-oxopropan-2-yl)-4- | |
| | (cyclohexylmethoxy)benzenesulfonamide | |
| 171-1 | N-((R)-3-((S)-3-aminopyrrolidin-1-yl)-1-(4′-chloro-[1,1′-biphenyl]-4-yl)- | B** |
| | 1,1-difluoro-3-oxopropan-2-yl)-4- | |
| | (cyclohexylmethoxy)benzenesulfonamide | |
| 172-2 | (S)-N-(3-(4-aminopiperidin-1-yl)-1-(4-chloro-3-isopropoxyphenyl)-1,1- | C** |
| | difluoro-3-oxopropan-2-yl)-6-(cyclopentyloxy)naphthalene-2-sulfonamide | |
| 172-1 | (R)-N-(3-(4-aminopiperidin-1-yl)-1-(4-chloro-3-isopropoxyphenyl)-1,1- | B** |
| | difluoro-3-oxopropan-2-yl)-6-(cyclopentyloxy)naphthalene-2-sulfonamide | |
| 174-1 | (R)-1-(3-(4′-chloro-[1,1′-biphenyl]-4-yl)-2-((6- | B** |
| | (cyclopentyloxy)naphthalene)-2-sulfonamido)-3,3-difluoropropanoyl)-N′- | |
| | hydroxypiperidine-4-carboximidamide | |
| 174-2 | (S)-1-(3-(4′-chloro-[1,1′-biphenyl]-4-yl)-2-((6- | D** |
| | (cyclopentyloxy)naphthalene)-2-sulfonamido)-3,3-difluoropropanoyl)-N′- | |
| | hydroxypiperidine-4-carboximidamide | |
| 161-3 | N-((R)-3-((S)-4-amino-3,3-difluoropiperidin-1-yl)-1-(4′-chloro-[1,1′- | A** |
| | biphenyl]-4-yl)-1,1-difluoro-3-oxopropan-2-yl)-6- | |
| | (cyclopentyloxy)naphthalene-2-sulfonamide | |
| 161-4 | N-((S)-3-((R)-4-amino-3,3-difluoropiperidin-1-yl)-1-(4′-chloro-[1,1′- | B** |
| | biphenyl]-4-yl)-1,1-difluoro-3-oxopropan-2-yl)-6- | |
| | (cyclopentyloxy)naphthalene-2-sulfonamide | |
| 175-2 | (S)-N-(3-(4-aminopiperidin-1-yl)-1-(4′-chloro-[1,1′-biphenyl]-4-yl)-1,1- | D** |
| | difluoro-3-oxopropan-2-yl)-2-(6-(cyclopentyloxy)benzo[b]thiophen-2-yl)- | |
| | 2-oxoacetamide | |
| 175-1 | (R)-N-(3-(4-aminopiperidin-1-yl)-1-(4′-chloro-[1,1′-biphenyl]-4-yl)-1,1- | C** |
| | difluoro-3-oxopropan-2-yl)-2-(6-(cyclopentyloxy)benzo[b]thiophen-2-yl)- | |
| | 2-oxoacetamide | |
| 173-1 | (R)-1-(3-(4′-chloro-[1,1′-biphenyl]-4-yl)-2-((6- | B** |
| | (cyclopentyloxy)naphthalene)-2-sulfonamido)-3,3- | |
| | difluoropropanoyl)piperidine-4-carboxamide | |
| 173-2 | (S)-1-(3-(4′-chloro-[1,1′-biphenyl]-4-yl)-2-((6- | D** |
| | (cyclopentyloxy)naphthalene)-2-sulfonamido)-3,3- | |
| | difluoropropanoyl)piperidine-4-carboxamide | |
| 176-1 | (R)-N-(1-(4′-chloro-[1,1′-biphenyl]-4-yl)-3-(4,6-dihydropyrrolo[3,4- | A** |
| | c]pyrazol-5(1H)-yl)-1,1-difluoro-3-oxopropan-2-yl)-6- | |
| | (cyclopentyloxy)naphthalene-2-sulfonamide | |
| 176-2 | (S)-N-(1-(4′-chloro-[1,1′-biphenyl]-4-yl)-3-(4,6-dihydropyrrolo[3,4- | D** |
| | c]pyrazol-5(1H)-yl)-1,1-difluoro-3-oxopropan-2-yl)-6- | |
| | (cyclopentyloxy)naphthalene-2-sulfonamide | |
| 177-2 | N-((2S)-3-(3-amino-8-azabicyclo[3.2.1]octan-8-yl)-1-(4-bromophenyl)- | C** |
| | 1,1-difluoro-3-oxopropan-2-yl)-6-cyclopropoxynaphthalene-2- | |
| | sulfonamide | |
| 177-1 | N-((2R)-3-(3-amino-8-azabicyclo[3.2.1]octan-8-yl)-1-(4-bromophenyl)- | B** |
| | 1,1-difluoro-3-oxopropan-2-yl)-6-cyclopropoxynaphthalene-2- | |
| | sulfonamide | |
| 178-1 | N-((R)-3-((3S,4S)-4-amino-3-fluoropiperidin-1-yl)-1-(4′-chloro-[1,1′- | B** |
| | biphenyl]-4-yl)-1,1-difluoro-3-oxopropan-2-yl)-6- | |
| | (cyclopentyloxy)naphthalene-2-sulfonamide | |
| 178-2 | N-((S)-3-((3S,4S)-4-amino-3-fluoropiperidin-1-yl)-1-(4′-chloro-[1,1′- | B** |
| | biphenyl]-4-yl)-1,1-difluoro-3-oxopropan-2-yl)-6- | |
| | (cyclopentyloxy)naphthalene-2-sulfonamide | |
| 178-3 | N-((S)-3-((3R,4R)-4-amino-3-fluoropiperidin-1-yl)-1-(4′-chloro-[1,1′- | D** |
| | biphenyl]-4-yl)-1,1-difluoro-3-oxopropan-2-yl)-6- | |
| | (cyclopentyloxy)naphthalene-2-sulfonamide | |
| 179-1 | N-((R)-1-(4′-chloro-[1,1′-biphenyl]-4-yl)-1,1-difluoro-3-((3aR,6aS)- | B** |
| | hexahydropyrrolo[3,4-c]pyrrol-2(1H)-yl)-3-oxopropan-2-yl)-4- | |
| | (cyclohexylmethoxy)benzenesulfonamide | |
| 179-2 | N-((S)-1-(4′-chloro-[1,1′-biphenyl]-4-yl)-1,1-difluoro-3-((3aR,6aS)- | D** |
| | hexahydropyrrolo[3,4-c]pyrrol-2(1H)-yl)-3-oxopropan-2-yl)-4- | |
| | (cyclohexylmethoxy)benzenesulfonamide | |
| 180-1 | N-((2R)-3-((2S)-2-amino-7-azabicyclo[2.2.1]heptan-7-yl)-1-(4′-chloro- | B** |
| | [1,1′-biphenyl]-4-yl)-1,1-difluoro-3-oxopropan-2-yl)-6- | |
| | (cyclopentyloxy)naphthalene-2-sulfonamide | |
| 180-2 | N-((2S)-3-((2S)-2-amino-7-azabicyclo[2.2.1]heptan-7-yl)-1-(4′-chloro- | C** |
| | [1,1′-biphenyl]-4-yl)-1,1-difluoro-3-oxopropan-2-yl)-6- | |
| | (cyclopentyloxy)naphthalene-2-sulfonamide | |
| 180-3 | N-((2S)-3-((2R)-2-amino-7-azabicyclo[2.2.1]heptan-7-yl)-1-(4′-chloro- | B** |
| | [1,1′-biphenyl]-4-yl)-1,1-difluoro-3-oxopropan-2-yl)-6- | |
| | (cyclopentyloxy)naphthalene-2-sulfonamide | |
| 180-4 | N-((2R)-3-((2R)-2-amino-7-azabicyclo[2.2.1]heptan-7-yl)-1-(4′-chloro- | B** |
| | [1,1′-biphenyl]-4-yl)-1,1-difluoro-3-oxopropan-2-yl)-6- | |
| | (cyclopentyloxy)naphthalene-2-sulfonamide | |
| 181-1 | (R)-N-(3-(4-aminopiperidin-1-yl)-1-(4′-chloro-[1,1′-biphenyl]-3-yl)-1,1- | B** |
| | difluoro-3-oxopropan-2-yl)-4-(cyclohexylmethoxy)benzenesulfonamide | |
| 181-2 | (S)-N-(3-(4-aminopiperidin-1-yl)-1-(4′-chloro-[1,1′-biphenyl]-3-yl)-1,1- | B** |
| | difluoro-3-oxopropan-2-yl)-4-(cyclohexylmethoxy)benzenesulfonamide | |
| 182-1 | (R)-N-(3-(4-aminopiperidin-1-yl)-1,1-difluoro-3-oxo-1-(4-(1- | A** |
| | (trifluoromethyl)cyclopropyl)phenyl)propan-2-yl)-6- | |
| | (cyclopentyloxy)naphthalene-2-sulfonamide | |
| 183-1 | N-((R)-3-((1R,5S,8R)-8-amino-3-azabicyclo[3.2.1]octan-3-yl)-1-(4′- | A** |
| | chloro-[1,1′-biphenyl]-4-yl)-1,1-difluoro-3-oxopropan-2-yl)-4- | |
| | (cyclohexylmethoxy)benzenesulfonamide | |
| 183-2 | N-((S)-3-((1R,5S,8S)-8-amino-3-azabicyclo[3.2.1]octan-3-yl)-1-(4′- | B** |
| | chloro-[1,1′-biphenyl]-4-yl)-1,1-difluoro-3-oxopropan-2-yl)-4- | |
| | (cyclohexylmethoxy)benzenesulfonamide | |
| 184-1 | N-((R)-3-((1R,5S,8R)-8-amino-3-azabicyclo[3.2.1]octan-3-yl)-1-(4′- | B** |
| | chloro-[1,1′-biphenyl]-4-yl)-1,1-difluoro-3-oxopropan-2-yl)-6- | |
| | (cyclopentyloxy)naphthalene-2-sulfonamide | |
| 184-2 | N-((S)-3-((1R,5S,8S)-8-amino-3-azabicyclo[3.2.1]octan-3-yl)-1-(4′- | B** |
| | chloro-[1,1′-biphenyl]-4-yl)-1,1-difluoro-3-oxopropan-2-yl)-6- | |
| | (cyclopentyloxy)naphthalene-2-sulfonamide | |
| 185-1 | (R)-N-(3-(4-aminopiperidin-1-yl)-1-(4′-chloro-[1,1′-biphenyl]-4-yl)-1,1- | C** |
| | difluoro-3-oxopropan-2-yl)-4-(2- | |
| | (cyclopentyloxy)ethyl)benzenesulfonamide | |
| 185-2 | (S)-N-(3-(4-aminopiperidin-1-yl)-1-(4′-chloro-[1,1′-biphenyl]-4-yl)-1,1- | D** |
| | difluoro-3-oxopropan-2-yl)-4-(2- | |
| | (cyclopentyloxy)ethyl)benzenesulfonamide | |
| 187-2 | (S)-N-(3-(4-aminopiperidin-1-yl)-1-(4-bromophenyl)-1,1-difluoro-3- | D** |
| | oxopropan-2-yl)-6-(cyclopentyloxy)-N-(2,2,2-trifluoroethyl)naphthalene- | |
| | 2-sulfonamide | |
| 187-1 | (R)-N-(3-(4-aminopiperidin-1-yl)-1-(4-bromophenyl)-1,1-difluoro-3- | D** |
| | oxopropan-2-yl)-6-(cyclopentyloxy)-N-(2,2,2-trifluoroethyl)naphthalene- | |
| | 2-sulfonamide | |
| 186-1 | N-((R)-3-((S)-3-aminopyrrolidin-1-yl)-1-(4-chlorophenyl)-1,1-difluoro-3- | B** |
| | oxopropan-2-yl)-4-(cyclohexylmethoxy)benzenesulfonamide | |
| 186-2 | N-((S)-3-((S)-3-aminopyrrolidin-1-yl)-1-(4-chlorophenyl)-1,1-difluoro-3- | D** |
| | oxopropan-2-yl)-4-(cyclohexylmethoxy)benzenesulfonamide | |
| 188-1 | (R)-N-(1-(4′-chloro-[1,1′-biphenyl]-4-yl)-1,1-difluoro-3-oxo-3-(4,7- | B** |
| | diazaspiro[2.5]octan-7-yl)propan-2-yl)-6-(cyclopentyloxy)naphthalene-2- | |
| | sulfonamide | |
| 188-2 | (S)-N-(1-(4′-chloro-[1,1′-biphenyl]-4-yl)-1,1-difluoro-3-oxo-3-(4,7- | D** |
| | diazaspiro[2.5]octan-7-yl)propan-2-yl)-6-(cyclopentyloxy)naphthalene-2- | |
| | sulfonamide | |
| 189-2 | N-((S)-3-((R)-3-aminopyrrolidin-1-yl)-1-(4-chlorophenyl)-1,1-difluoro-3- | C** |
| | oxopropan-2-yl)-4-(cyclohexylmethoxy)benzenesulfonamide | |
| 189-1 | N-((R)-3-((R)-3-aminopyrrolidin-1-yl)-1-(4-chlorophenyl)-1,1-difluoro-3- | B** |
| | oxopropan-2-yl)-4-(cyclohexylmethoxy)benzenesulfonamide | |
| 190-2 | N-((S)-1-(4′-chloro-2-methoxy-[1,1′-biphenyl]-4-yl)-1,1-difluoro-3- | D** |
| | ((3aR,6aS)-hexahydropyrrolo[3,4-c]pyrrol-2(1H)-yl)-3-oxopropan-2-yl)- | |
| | 4-(cyclohexylmethoxy)benzenesulfonamide | |
| 190-1 | N-((R)-1-(4′-chloro-2-methoxy-[1,1′-biphenyl]-4-yl)-1,1-difluoro-3- | B** |
| | ((3aR,6aS)-hexahydropyrrolo[3,4-c]pyrrol-2(1H)-yl)-3-oxopropan-2-yl)- | |
| | 4-(cyclohexylmethoxy)benzenesulfonamide | |
| 191-1 | (R)-N-(1-(4′-chloro-[1,1′-biphenyl]-4-yl)-1,1-difluoro-3-oxo-3-(1,6- | A** |
| | diazaspiro[3.3]heptan-1-yl)propan-2-yl)-6-(cyclopentyloxy)naphthalene-2- | |
| | sulfonamide | |
| 191-2 | (S)-N-(1-(4′-chloro-[1,1′-biphenyl]-4-yl)-1,1-difluoro-3-oxo-3-(1,6- | B** |
| | diazaspiro[3.3]heptan-1-yl)propan-2-yl)-6-(cyclopentyloxy)naphthalene-2- | |
| | sulfonamide | |
| 192-1 | N-((R)-1-(4′-chloro-[1,1′-biphenyl]-4-yl)-1,1-difluoro-3-((3aS,6aS)- | B** |
| | hexahydropyrrolo[3,4-b]pyrrol-1(2H)-yl)-3-oxopropan-2-yl)-6- | |
| | (cyclopentyloxy)naphthalene-2-sulfonamide | |
| 192-2 | N-((S)-1-(4′-chloro-[1,1′-biphenyl]-4-yl)-1,1-difluoro-3-((3aR,6aR)- | C** |
| | hexahydropyrrolo[3,4-b]pyrrol-1(2H)-yl)-3-oxopropan-2-yl)-6- | |
| | (cyclopentyloxy)naphthalene-2-sulfonamide | |
| 192-4 | N-((S)-1-(4′-chloro-[1,1′-biphenyl]-4-yl)-1,1-difluoro-3-((3aS,6aS)- | D** |
| | hexahydropyrrolo[3,4-b]pyrrol-1(2H)-yl)-3-oxopropan-2-yl)-6- | |
| | (cyclopentyloxy)naphthalene-2-sulfonamide | |
| 192-3 | N-((R)-1-(4′-chloro-[1,1′-biphenyl]-4-yl)-1,1-difluoro-3-((3aR,6aR)- | A** |
| | hexahydropyrrolo[3,4-b]pyrrol-1(2H)-yl)-3-oxopropan-2-yl)-6- | |
| | (cyclopentyloxy)naphthalene-2-sulfonamide | |
| 194-2 | N-((2S)-3-(3-amino-8-azabicyclo[3.2.1]octan-8-yl)-1-(5-chlorothiophen-2- | D** |
| | yl)-1,1-difluoro-3-oxopropan-2-yl)-4- | |
| | (cyclohexylmethoxy)benzenesulfonamide | |
| 194-1 | N-((2R)-3-(3-amino-8-azabicyclo[3.2.1]octan-8-yl)-1-(5-chlorothiophen- | B** |
| | 2-yl)-1,1-difluoro-3-oxopropan-2-yl)-4- | |
| | (cyclohexylmethoxy)benzenesulfonamide | |
| 193-1 | (R)-N-(3-(4-aminopiperidin-1-yl)-1,1-difluoro-3-oxo-1-(4-(3,3,3- | A** |
| | trifluoroprop-1-en-2-yl)phenyl)propan-2-yl)-4- | |
| | (cyclohexylmethoxy)benzenesulfonamide | |
| 193-2 | (S)-N-(3-(4-aminopiperidin-1-yl)-1,1-difluoro-3-oxo-1-(4-(3,3,3- | D** |
| | trifluoroprop-1-en-2-yl)phenyl)propan-2-yl)-4- | |
| | (cyclohexylmethoxy)benzenesulfonamide | |
| 195-1 | N-((R)-1-(4′-chloro-[1,1′-biphenyl]-4-yl)-1,1-difluoro-3-((3aS,6aS)- | B** |
| | hexahydropyrrolo[3,4-b]pyrrol-1(2H)-yl)-3-oxopropan-2-yl)-4- | |
| | (cyclohexylmethoxy)benzenesulfonamide | |
| 195-2 | N-((S)-1-(4′-chloro-[1,1′-biphenyl]-4-yl)-1,1-difluoro-3-((3aR,6aR)- | D** |
| | hexahydropyrrolo[3,4-b]pyrrol-1(2H)-yl)-3-oxopropan-2-yl)-4- | |
| | (cyclohexylmethoxy)benzenesulfonamide | |
| 195-3 | N-((R)-1-(4′-chloro-[1,1′-biphenyl]-4-yl)-1,1-difluoro-3-((3aR,6aR)- | B** |
| | hexahydropyrrolo[3,4-b]pyrrol-1(2H)-yl)-3-oxopropan-2-yl)-4- | |
| | (cyclohexylmethoxy)benzenesulfonamide | |
| 195-4 | N-((S)-1-(4′-chloro-[1,1′-biphenyl]-4-yl)-1,1-difluoro-3-((3aS,6aS)- | D** |
| | hexahydropyrrolo[3,4-b]pyrrol-1(2H)-yl)-3-oxopropan-2-yl)-4- | |
| | (cyclohexylmethoxy)benzenesulfonamide | |
| 196-2 | (S)-N-(3-(4-aminopiperidin-1-yl)-1,1-difluoro-3-oxo-1-(4-(1- | C** |
| | (trifluoromethyl)cyclopropyl)phenyl)propan-2-yl)-4- | |
| | (cyclohexylmethoxy)benzenesulfonamide | |
| 196-1 | (R)-N-(3-(4-aminopiperidin-1-yl)-1,1-difluoro-3-oxo-1-(4-(1- | B** |
| | (trifluoromethyl)cyclopropyl)phenyl)propan-2-yl)-4- | |
| | (cyclohexylmethoxy)benzenesulfonamide | |
| 197-1 | N-((R)-1-(4′-chloro-2-methoxy-[1,1′-biphenyl]-4-yl)-1,1-difluoro-3- | A** |
| | ((3aR,6aS)-hexahydropyrrolo[3,4-c]pyrrol-2(1H)-yl)-3-oxopropan-2-yl)- | |
| | 6-(cyclopentyloxy)naphthalene-2-sulfonamide | |
| 197-2 | N-((S)-1-(4′-chloro-2-methoxy-[1,1′-biphenyl]-4-yl)-1,1-difluoro-3- | C** |
| | ((3aR,6aS)-hexahydropyrrolo[3,4-c]pyrrol-2(1H)-yl)-3-oxopropan-2-yl)- | |
| | 6-(cyclopentyloxy)naphthalene-2-sulfonamide | |
| 182-2 | (S)-N-(3-(4-aminopiperidin-1-yl)-1,1-difluoro-3-oxo-1-(4-(1- | C** |
| | (trifluoromethyl)cyclopropyl)phenyl)propan-2-yl)-6- | |
| | (cyclopentyloxy)naphthalene-2-sulfonamide | |
| 198-2 | (S)-N-(3-(4-aminopiperidin-1-yl)-1-(7-chlorodibenzo[b,d]furan-3-yl)-1,1- | D** |
| | difluoro-3-oxopropan-2-yl)-4-(cyclohexylmethoxy)benzenesulfonamide | |
| 198-1 | (R)-N-(3-(4-aminopiperidin-1-yl)-1-(7-chlorodibenzo[b,d]furan-3-yl)-1,1- | B** |
| | difluoro-3-oxopropan-2-yl)-4-(cyclohexylmethoxy)benzenesulfonamide | |

### Twitcher Mouse Model

The twitcher is a naturally-occurring mouse mutant caused by an abnormality in the gene coded for galactosylceramidase (Kobayashi et al., Brain Res. 202: 479-483 (1980); Suzuki and Suzuki, Am J Path. 111: 394-397 (1983)). It is therefore genetically equivalent to human globoid cell leukodystrophy (Krabbe disease). Affected mice develop clinical symptoms at the onset of the active myelination period and, if untreated, die by 35+ days. The pathology is very similar to that in human disease. Toxicity of galactosylsphingosine (psychosine) that accumulates abnormally in the nervous system is considered to be primarily responsible for the pathogenesis.

To evaluate the potential efficacy of different compounds described herein in Krabbe disease, the Twitcher mouse model was used. This model is as described in Hawkins-Salsbury et al., J Neurosci. 35(16): 6495-6505 (2015), which is incorporated herein by reference in its entirety.

If tested compounds are found which have a marked effect on the experimental endpoints (e.g., life span with improved motor function, GALC expression, psychosine levels, and neuroinflammation), an additional experiment is performed looking at effects on activity, inverted screen, and bar crossing tests, as well as average survival time, compared to vehicle-treated (control) mice.

Other objects, features and advantages of the compounds, methods and compositions described herein will become apparent from the following description. It should be understood, however, that the description and the specific examples, while indicating specific embodiments, are given by way of illustration only, since various changes and modifications within the spirit and scope of the present description will become apparent from this detailed description.

All publications including patents, patent applications and published patent applications cited herein are hereby incorporated by reference for all purposes.

We claim:

1. A compound of Formula I: 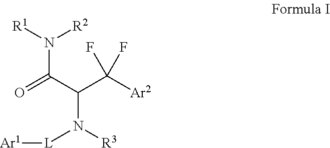
where
R¹ is alkyl, aminoalkyl, alkylaminoalkyl, or dialkylaminoalkyl;
R² is alkyl, aminoalkyl, alkylaminoalkyl, or dialkylaminoalkyl;
or R¹ and R² together with the nitrogen to which they are attached form a 4-8 membered heterocycloalkyl ring optionally substituted with 1, 2, or 3 R¹⁰ groups; or a bicyclic heteroaryl ring with 8 ring atoms, where 1-3 ring atoms are nitrogen;
R³ is H, alkyl, or haloalkyl;
L is S(O)_{q}, C₁₋₃alkylene-S(O)_{q}, C(O)—C(O), or C*H(OH)C(O), wherein the alkylene in the C₁-3alkylene-S(O) q forms a covalent bond with Ar¹, and wherein the “C*” in the C*H(OH)C(O) forms a covalent bond with Ar¹;
q is 0, 1, or 2;
Ar¹ is aryl or heteroaryl, each optionally substituted with 1 or 2 R¹¹ groups;
Ar² is aryl or heteroaryl, each optionally substituted with 1 or 2 R¹² groups;
each R¹⁰ is independently halo, cyano, nitro, amino, alkylamino, dialkylamino, aminoalkyl, alkylaminoalkyl, dialkylaminoalkyl, aminocarbonyl, alkylaminocarbonyl, dialkylaminocarbonyl, or C(═NOR^{10a})(NR^{10b}R^{10c});
R^{11a}, R^{10b}, and R^{10c} are each independently H or alkyl;
each R¹¹ is independently halo, cyano, nitro, alkyl, alkoxy, hydroxy, cycloalkyl, (cycloalkyl)alkyl, heterocycloalkyl, (heterocycloalkyl)alkyl, cycloalkyloxy, heterocycloalkyloxy, (cycloalkyl)alkyloxy, cycloalkyloxyalkyl, aryl, aryloxy, arylalkyloxy, heteroaryl, heteroaryloxy, or heteroarylalkyloxy; wherein each aryl and heteroaryl, whether alone or as part of another group, are each independently optionally substituted with halo, alkyl, haloalkyl, or alkoxy;
each R¹² is independently halo, cyano, nitro, hydroxy, alkyl, haloalkyl, haloalkenyl, alkoxy, cycloalkyloxy, cycloalkyl, heterocycloalkyl, cycloalkyl substituted with haloalkyl, (cycloalkyl)alkyloxy, heterocycloalkyloxy, (heterocycloalkyl)alkyloxy, aryl, heteroaryl, aryloxy, arylalkyloxy, heteroaryloxy, or heteroarylalkyloxy; wherein each aryl and heteroaryl, whether alone or as part of another group, are each independently optionally substituted with 1 or 2 groups selected from halo, alkyl, haloalkyl, and alkoxy; and
optionally a single stereoisomer or mixture of stereoisomers thereof and additionally optionally a pharmaceutically acceptable salt thereof.

2. The compound of claim 1, wherein the compound of Formula (I) is according to Formula I(a): 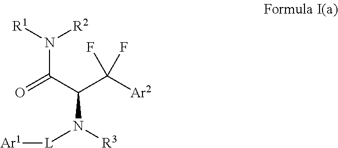
and optionally a single stereoisomer or mixture of stereoisomers thereof and additionally optionally a pharmaceutically acceptable salt thereof.

3. The compound of claim 1, wherein the compound of Formula (I) is according to Formula I(b): 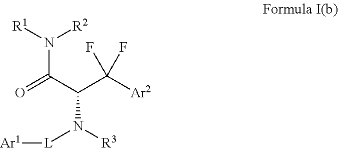
and optionally a single stereoisomer or mixture of stereoisomers thereof and additionally optionally a pharmaceutically acceptable salt thereof.

4. The compound or salt of any one of claims 1-3, wherein L is S(O)_{q} and q is 0, 1, or 2.

5. The compound or salt of any one of claims 1-3, wherein L is C(O)—C(O).

6. The compound or salt of any one of claims 1-3, wherein L is C*H(OH)C(O), wherein “C*” forms a covalent bond with Ar¹.

7. The compound or salt of claim 1, wherein the compound of Formula (I) is according to Formula I(c), Formula I(d), Formula I(e), or Formula I(f):

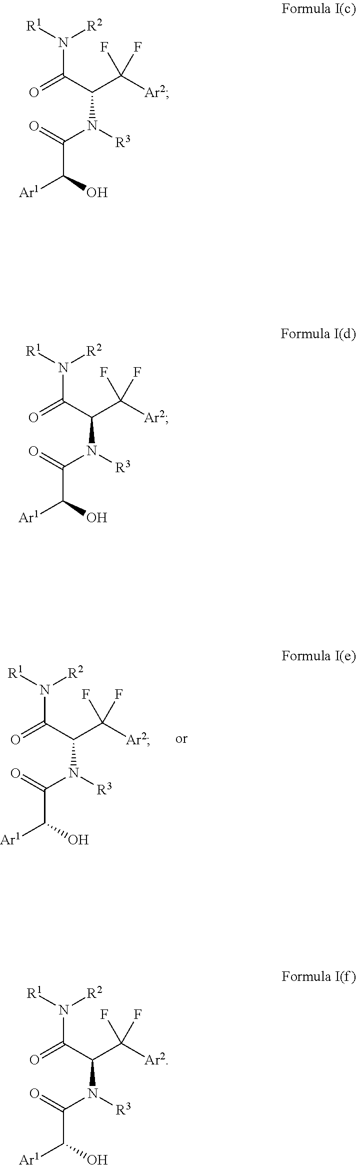

8. The compound or salt of any one of claims 1-7, wherein Ar¹ is naphthyl optionally substituted with 1 or 2 R¹¹ groups.

9. The compound or salt of any one of claims 1-7, wherein Ar¹ is phenyl optionally substituted with 1 or 2 R¹¹ groups.

10. The compound or salt of any one of claims 1-7, wherein Ar¹ is heteroaryl optionally substituted with 1 or 2 R¹¹ groups.

11. The compound or salt of any one of claims 1-7, wherein Ar¹ is a 5-6 membered heteroaryl or a 9-10 membered bicyclic heteroaryl, each containing 1 or 2 ring heteroatoms, wherein each is optionally substituted with 1 or 2 R¹¹ groups.

12. The compound or salt of any one of claims 1-7, wherein Ar¹ is a 5-6 membered heteroaryl containing 1 ring heteroatom, wherein the heteroaryl is optionally substituted with 1 or 2 R¹¹ groups.

13. The compound or salt of any one of claims 1-7, wherein Ar¹ is a 9-10 membered bicyclic heteroaryl containing 1 or 2 ring heteroatoms, wherein the heteroaryl is optionally substituted with 1 or 2 R¹¹ groups.

14. The compound or salt of any one of claims 1-7, wherein Ar¹ is 2-pyridyl, 3-pyridyl, indazolyl, thienyl, or benzothienyl, wherein each is optionally substituted with 1 or 2 R¹¹ groups.

15. The compound or salt of any one of claims 1-14, wherein each R¹¹ is independently halo, alkyl, alkoxy, hydroxy, cycloalkyl, (cycloalkyl)alkyl, heterocycloalkyl, (heterocycloalkyl)alkyl, cycloalkyloxy, heterocycloalkyloxy, (cycloalkyl)alkyloxy, cycloalkyloxyalkyl, aryl, aryloxy, arylalkyloxy, heteroaryl, heteroaryloxy, or heteroarylalkyloxy; wherein each aryl and heteroaryl, whether alone or as part of another group, are each independently optionally substituted with halo, alkyl, haloalkyl, or alkoxy.

16. The compound or salt of any one of claims 1-14, wherein each R¹¹ is independently halo, alkyl, alkoxy, (cycloalkyl)alkyl, cycloalkyloxy, (cycloalkyl)alkyloxy, cycloalkyloxyalkyl, aryl, aryloxy, arylalkyloxy, or heteroaryloxy; wherein each aryl and heteroaryl, whether alone or as part of another group, are each independently optionally substituted with halo.

17. The compound or salt of any one of claims 1-14, wherein each R¹¹ is independently fluoro, chloro, bromo, iodo, methyl, ethyl, n-propyl, i-propyl, methoxy, ethoxy, propoxy, i-propoxy, butoxy, i-butoxy, sec-butoxy, cyclopropyloxy, cyclobutyloxy, cyclopentyloxy, cyclohexyloxy, pyridyloxy, or benzyloxy.

18. The compound or salt of any one of claims 1-7, wherein Ar¹ is phenyl substituted with 1 or 2 R¹¹ groups; wherein each R¹¹ is independently alkoxy, (cycloalkyl)alkyloxy, cycloalkyloxyalkyl, aryl, aryloxy, arylalkyloxy, or heteroaryloxy, wherein each aryl and heteroaryl, whether alone or as part of another group, are each independently optionally substituted with halo.

19. The compound or salt of any one of claims 1-14, wherein each R¹¹ is independently selected from methoxy, ethoxy, propoxy, i-butoxy, 2-ethylbutoxy, butoxy, hexyloxy, cyclopentylmethyloxy, cyclohexylmethyloxy, cyclohexylethyloxy, cyclopentyloxyethyl, 4-fluorophenyl, 4-fluorophenoxy, benzyloxy, 3-fluorobenzyloxy, 4-fluorobenzyloxy, 4-fluoro-2-pyridyloxy, or 4-chloro-2-pyridyloxy.

20. The compound or salt of any one of claims 1-7, wherein Ar¹ is naphthyl substituted with 1 or 2 R¹¹ groups, wherein each R¹¹ is independently halo, alkyl, alkoxy, cycloalkyloxy, arylalkyloxy, or heteroaryloxy.

21. The compound or salt of any one of claims 1-14 or 20, wherein each R¹¹ is independently fluoro, chloro, bromo, iodo, methyl, ethyl, n-propyl, i-propyl, methoxy, ethoxy, propoxy, i-propoxy, butoxy, i-butoxy, sec-butoxy, cyclopropyloxy, cyclobutyloxy, cyclopentyloxy, cyclohexyloxy, pyridyloxy, or benzyloxy.

22. The compound or salt of any one of claims 1-7, wherein Ar¹ is naphthyl substituted with 1 or 2 R¹¹ groups, wherein each R¹¹ is independently alkyl, alkoxy, cycloalkyloxy, arylalkyloxy, or heteroaryloxy.

23. The compound or salt of any one of claims 1-14 or 22, wherein each R¹¹ is independently methyl, ethyl, n-propyl, i-propyl, methoxy, ethoxy, propoxy, i-propoxy, butoxy, i-butoxy, sec-butoxy, cyclopropyloxy, cyclobutyloxy, cyclopentyloxy, cyclohexyloxy, pyridyloxy, or benzyloxy.

24. The compound or salt of any one of claims 1-7, wherein Ar¹ is 2-pyridyl, 3-pyridyl, indazolyl, thienyl, or benzothienyl, wherein each Ar¹ is independently substituted with 1 R¹¹ selected from (cycloalkyl)alkyl, cycloalkyloxy, cycloalkyloxyalkyl, and aryl optionally substituted with halo.

25. The compound or salt of any one of claims 1-14 or 24, wherein R¹¹ is 4-fluorophenyl, cyclopentylmethyl, cyclopentyloxymethyl, or cyclopentyloxy.

26. The compound or salt of any one of claims 1-25, wherein R¹ and R² together with the nitrogen to which they are attached form a 4-8 membered heterocycloalkyl ring which is optionally substituted with 1, 2, or 3 R¹⁰ groups.

27. The compound or salt of any one of claims 1-25, wherein R¹ and R² together with the nitrogen to which they are attached form a 4-6 membered monocyclic heterocycloalkyl ring which is optionally substituted with 1, 2, or 3 R¹⁰ groups.

28. The compound or salt of any one of claims 1-25, wherein R¹ and R² together with the nitrogen to which they are attached form a 7-8 membered bicyclic heterocycloalkyl ring which is optionally substituted with 1, 2, or 3 R¹⁰ groups.

29. The compound or salt of any one of claims 1-25, wherein R¹ and R² together with the nitrogen to which they are attached form a 8 membered heterocycloalkyl ring which is optionally substituted with 1, 2, or 3 R¹⁰ groups.

30. The compound or salt of any one of claims 1-25, wherein R¹ and R² are each independently alkyl, aminoalkyl, alkylaminoalkyl, or dialkylaminoalkyl.

31. The compound or salt of any one of claims 1-29, wherein each R¹⁰ is independently halo, cyano, amino, alkylamino, dialkylamino, aminoalkyl, alkylaminoalkyl, dialkylaminoalkyl, aminocarbonyl, alkylaminocarbonyl, dialkylaminocarbonyl, or C(═NOH)(NH₂).

32. The compound or salt of any one of claims 1-29, wherein each R¹⁰ is independently halo, amino, alkylamino, or dialkylamino.

33. The compound or salt of any one of claims 1-32, wherein Ar² is phenyl or naphthyl, wherein each is optionally substituted with 1 or 2 R¹² groups.

34. The compound or salt of any one of claims 1-32, wherein Ar² is heteroaryl optionally substituted with 1 or 2 R¹² groups.

35. The compound or salt of any one of claims 1-32, wherein Ar² is a 5-6 membered heteroaryl or a 9-10 membered bicyclic heteroaryl, wherein each heteroaryl is optionally substituted with 1 or 2 R¹² groups.

36. The compound or salt of any one of claims 1-32, wherein Ar² is a 5-6 membered heteroaryl or a 9-10 membered bicyclic heteroaryl, each containing 1 ring heteroatom, wherein each heteroaryl is optionally substituted with 1 or 2 R¹² groups.

37. The compound or salt of any one of claims 1-32, wherein Ar² is a 5-6 membered heteroaryl containing 1 ring heteroatom, wherein the heteroaryl is optionally substituted with 1 or 2 R¹² groups.

38. The compound or salt of any one of claims 1-32, wherein Ar² is a 9-10 membered bicyclic heteroaryl containing 1 ring heteroatom, wherein the heteroaryl is optionally substituted with 1 or 2 R¹² groups.

39. The compound or salt of any one of claims 1-32, wherein Ar² is 2-pyridyl, thienyl, or dibenzofuranyl, wherein each is optionally substituted with 1 or 2 R¹² groups.

40. The compound or salt of any one of claims 1-39, wherein Ar² is substituted with 1 or 2 R¹² groups.

41. The compound or salt of any one of claims 1-40, wherein R¹² is halo, hydroxy, alkyl, haloalkyl, haloalkenyl, alkoxy, cycloalkyloxy, cycloalkyl, heterocycloalkyl, cycloalkyl substituted with haloalkyl, (cycloalkyl)alkyloxy, heterocycloalkyloxy, (heterocycloalkyl)alkyloxy, aryl, heteroaryl, aryloxy, arylalkyloxy, heteroaryloxy, or heteroarylalkyloxy; wherein each aryl and heteroaryl, whether alone or as part of another group, are each independently optionally substituted with 1 or 2 groups selected from halo, alkyl, haloalkyl, and alkoxy.

42. The compound or salt of any one of claims 1-40, wherein R¹² is halo, hydroxy, alkyl, haloalkyl, haloalkenyl, alkoxy, cycloalkyloxy, cycloalkyl substituted with haloalkyl, (heterocycloalkyl)alkyloxy, aryl, heteroaryl, aryloxy, or arylalkyloxy; wherein each aryl and heteroaryl, whether alone or as part of another group, are each independently optionally substituted with 1 or 2 groups selected from halo and haloalkyl.

43. The compound or salt of any one of claims 1-40, wherein R¹² is fluoro, chloro, bromo, iodo, hydroxy, methoxy, ethoxy, propoxy, i-propoxy, butoxy, cyclopropyloxy, cyclopentyloxy, cyclohexyloxy, trifluoromethyl, 3,3,3-trifluoropropenyl, (trifluromethyl)cyclopropyl, phenyl, chlorophenyl, trifluoromethylphenyl, 4-chlorophenoxy, (piperidinyl)ethoxy, (morpholinyl)ethoxy, 2-chlorothiophene, phenoxy, or phenylmethoxy.

44. The compound or salt of any one of claims 1-32, wherein Ar² is phenyl substituted with 1 or 2 R¹² groups, wherein each R¹² is independently halo, hydroxy, haloalkyl, haloalkenyl, alkoxy, cycloalkyloxy, cycloalkyl substituted with haloalkyl, (heterocycloalkyl)alkyloxy, aryl, heteroaryl, aryloxy, or arylalkyloxy; wherein each aryl and heteroaryl, whether alone or as part of another group, are each independently optionally substituted with 1 or 2 groups selected from halo and haloalkyl.

45. The compound or salt of any one of claims 1-32, wherein Ar² is phenyl substituted with 2 R¹² groups, wherein each R¹² is independently halo, alkoxy, cycloalkyloxy, phenyl or heteroaryl, wherein the phenyl and heteroaryl are each optionally substituted with 1 or 2 halo.

46. The compound or salt of any one of claims 1-39, wherein Ar² is unsubstituted.

47. The compound or salt of any one of claims 1-3, wherein L is C(O)—C(O), R³ is H, and Ar¹ is aryl or heteroaryl, wherein the aryl and heteroaryl are each independently substituted with 1 R¹¹ group, wherein R¹¹ is alkoxy, cycloalkyloxy, (cycloalkyl)alkyloxy, or aryl optionally substituted with halo.

48. The compound or salt of any one of claims 1-14 or 47, wherein R¹¹ is i-propoxy, cyclopentyloxy, fluorophenyl, or cyclohexylmethoxy.

49. The compound or salt of any one of claims 1-25, wherein R¹ and R² together with the nitrogen to which they are attached form a 6-8 membered heterocycloalkyl ring substituted with an amino, alkylamino, or dialkylamino group.

50. The compound or salt of any one of claims 1, 7-26, 28-29, or 31-46, having the structure:

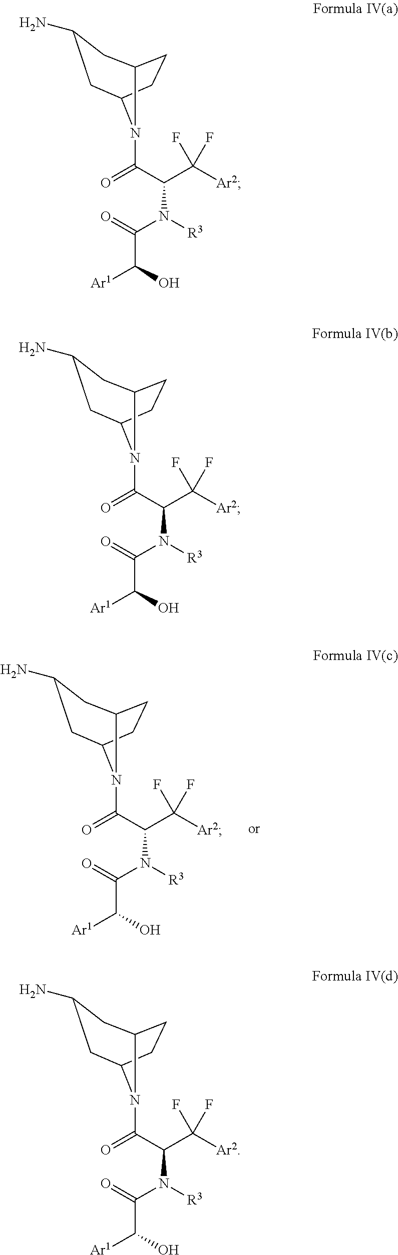

51. A compound selected from Table 1; optionally as a tautomer, a single stereoisomer or mixture of stereoisomers thereof and additionally optionally as a pharmaceutically acceptable salt thereof.

52. A compound selected from Compounds 1-200, or a single stereoisomer or mixture of stereoisomers thereof and additionally optionally as a pharmaceutically acceptable salt thereof.

53. A compound selected from compounds 5-1, 28-1, 29-1, 32-1, 33-1, 34-1, 35-1, 36-1, 37-1, 42-1, 43-1, 44-1, 48-1, 50-1, 52-1, 54-1, 55-1, 56-1, 57-1, 59-1, 66-1, 61-1, 61-2, 71-1, 73-1, 80-1, 82-1, 83-1, 87-1, 86-1, 89-1, 90-1, 91-1, 92-1, 95-1, 100-1, 101-1, 102-1, 103-1, 105-1, 107-1, 108-1, 111-1, 112-1, 113-1, 118-1, 120-1, 121-1, 123-1, 124-1, 125-1, 127-1, 126-1, 128-1, 135-1, 138-1, 139-1, 142-1, 144-1, 145-1, 146-1, 147-1, 149-1, 150-1, 155-1, 169-1, 161-3, 176-1, 182-1, 183-1, 191-1, 192-3, 193-1, and 197-1, or a single stereoisomer or mixture of stereoisomers thereof and additionally optionally as a pharmaceutically acceptable salt thereof.

54. A compound selected from compounds 2, 3, 11, 12, 15, 17, 18, 20, 22, 23, 25, 26, 29, 30, 35, 39, 40, 50, 58, 59, 60, 63, 64, 70, 72, 79, 81, 85, 84, 96, 103, 104, 105, 109, 113, 117, 120, 125, 127, 128, 129, 134, 141, 142, 145, 146, 151, 152, 153, 155, 156, 163, 171, 179, 181, 183, 185, 186, 189, 190, 194, 193, 195, 196, and 198, or a single stereoisomer or mixture of stereoisomers thereof and additionally optionally as a pharmaceutically acceptable salt thereof.

55. A compound selected from compounds 1, 4, 5, 6, 7, 8, 9, 10D, 13, 16, 19, 21, 24, 5, 27, 28, 31, 32, 33, 34, 36, 37, 38, 41, 42, 43, 44, 45, 46, 47, 48, 49, 51, 52, 53, 54, 55, 56, 57, 62, 66, 65, 67, 68, 61, 69, 71, 73, 76, 80, 82, 83, 87, 86, 89, 90, 91, 97, 98, 100, 101, 102, 106, 107, 108, 110, 111, 112, 114, 115, 116, 118, 119, 121, 122, 123, 124, 126, 130, 131, 132, 133, 135, 136-cis, 136-trans, 137, 138, 139, 140, 143, 144, 147, 148, 200, 149, 150, 154, 157, 158, 159, 160, 161, 162, 166, 168, 169, 170, 172, 174, 173, 176, 177, 178, 180, 182-1, 182-2, 184, 187, 188, 191, 192, 197-1, and 197-2, or a single stereoisomer or mixture of stereoisomers thereof and additionally optionally as a pharmaceutically acceptable salt thereof.

56. A compound selected from compounds 199, 14, 75, 74, 77, 78, 88, 92, 93, 94, 95, 99, 164, 165, 167, and 175, or a single stereoisomer or mixture of stereoisomers thereof and additionally optionally as a pharmaceutically acceptable salt thereof.

57. A compound selected from compounds 1, 2, 3, 4, 5, 6, 7, 8, 10, 11, 12, 13, 199, 15, 16, 17, 18, 20, 21, 22, 23, 24, 25, 26, 27, 28, 29, 30, 31, 32, 33, 34, 35, 36, 37, 38, 39, 40, 42, 43, 44, 45, 46, 47, 48, 49, 50, 51, 52, 53, 54, 55, 56, 57, 58, 59, 60, 62, 63, 64, 66, 65, 67, 68, 61, 69, 70, 75, 71, 72, 73, 74, 76, 77, 78, 79, 80, 81, 82, 83, 85, 84, 87, 86, 88, 89, 90, 91, 92 93, 94, 95, 96, 97, 98, 99, 100, 101, 102, 103, 104, 105, 106, 107, 109, 108, 110, 111, 112, 113, 114, 115, 116, 117, 118, 119, 120, 121, 122, 123, 124, 125, 127, 126, 128, 129, 130, 131, 132, 135, 137, 138, 139, 140, 142, 143, 144, 145, 146, 147, 148, 200, 149, 150, 151, 153, 154, 155, 156, 157, 161, 162, 163, 164, 165, 166, 167, 168, 169, 170, 171, 172, 174, 173, 176, 177, 178, 179, 180, 181, 182-1, 182-2, 183, 184, 185, 187, 186, 188, 189, 190, 191, 192, 194, 193, 195, 196, 197-1, 197-2, and 198, or a single stereoisomer or mixture of stereoisomers thereof and additionally optionally as a pharmaceutically acceptable salt thereof.

58. A compound selected from compounds 9, 14, 133, 134, 160, and 175, or a single stereoisomer or mixture of stereoisomers thereof and additionally optionally as a pharmaceutically acceptable salt thereof.

59. A compound selected from compounds 19, 41, 136-cis, 136-trans, 141, 152, 158, and 159, or a single stereoisomer or mixture of stereoisomers thereof and additionally optionally as a pharmaceutically acceptable salt thereof.

60. A pharmaceutical composition comprising a compound of any one of claims 1-59 and a pharmaceutically acceptable excipient.

61. A method of treating lysosomal storage disease with a compound of any one of claims 1-59 or the pharmaceutical composition of claim 60.

62. The method of claim 61, wherein the lysosomal storage disease is Krabbe disease or Metachromatic Leukodystrophy.

63. The compound of any one of claims 1-59 or the pharmaceutical composition of claim 60 for use as a medicament.

64. The compound of any one of claims 1-59 or the pharmaceutical composition of claim 60 for use in a method of treating a lysosomal storage disease.

65. The compound for use or the pharmaceutical composition for use of claim 64, wherein the lysosomal storage disease is Krabbe disease or Metachromatic Leukodystrophy.
